SEQUENCE LISTING (826 sequences)

SEQ ID NO: 1 - protein, 5 aa, Artificial Sequence
  source: /note=“Description of Artificial Sequence: Synthetic peptide”
RYRMS

SEQ ID NO: 2 - protein, 17 aa, Artificial Sequence
  source: /note=“Description of Artificial Sequence: Synthetic peptide”
RISRSGGATAYADSVKG

SEQ ID NO: 3 - protein, 12 aa, Artificial Sequence
  source: /note=“Description of Artificial Sequence: Synthetic peptide”
DATGRHYNGMDV

SEQ ID NO: 4 - protein, 11 aa, Artificial Sequence
  source: /note=“Description of Artificial Sequence: Synthetic peptide”
RASQTITRHLN

SEQ ID NO: 5 - protein, 7 aa, Artificial Sequence
  source: /note=“Description of Artificial Sequence: Synthetic peptide”
GTSALQT

SEQ ID NO: 6 - protein, 9 aa, Artificial Sequence
  source: /note=“Description of Artificial Sequence: Synthetic peptide”
QQSHTKPWT

SEQ ID NO: 7 - protein, 30 aa, Artificial Sequence
  source: /note=“Description of Artificial Sequence: Synthetic polypeptide”
QVQLLQSGGGLVQPGGSLRLSCAASGFTFS

SEQ ID NO: 8 - protein, 14 aa, Artificial Sequence
  source: /note=“Description of Artificial Sequence: Synthetic peptide”
WVRQAPGKGLEWVS

SEQ ID NO: 9 - protein, 32 aa, Artificial Sequence
  source: /note=“Description of Artificial Sequence: Synthetic polypeptide”
RFTISRDNSKNTLYLQMNSLRAEDTAVYYCAR

SEQ ID NO: 10 - protein, 11 aa, Artificial Sequence
  source: /note=“Description of Artificial Sequence: Synthetic peptide”
WGRGTLVTVSS

SEQ ID NO: 11 - protein, 23 aa, Artificial Sequence
  source: /note=“Description of Artificial Sequence: Synthetic peptide”
EIALTQSPSSLSASVGDRVTITC

SEQ ID NO: 12 - protein, 15 aa, Artificial Sequence
  source: /note=“Description of Artificial Sequence: Synthetic peptide”
WFQQKPGKAPNLLIH

SEQ ID NO: 13 - protein, 32 aa, Artificial Sequence
  source: /note=“Description of Artificial Sequence: Synthetic polypeptide”
GVPPRFSGGGSGTDFTLTINSLQPEDFGTYYC

SEQ ID NO: 14 - protein, 13 aa, Artificial Sequence
  source: /note=“Description of Artificial Sequence: Synthetic peptide”
FGPGTKVEIKRTV

SEQ ID NO: 15 - protein, 121 aa, Artificial Sequence
  source: /note=“Description of Artificial Sequence: Synthetic polypeptide”
QVQLLQSGGGLVQPGGSLRLSCAASGFTFSRYRMSWVRQAPGKGLEWVSRISRSGGATAYADSVKGRFTISRDNSKNTLYLQMNSLRAEDTAVYYCARDATGRHYNGMDVWGRGTLVTVSS

SEQ ID NO: 16 - protein, 110 aa, Artificial Sequence
  source: /note=“Description of Artificial Sequence: Synthetic polypeptide”
EIALTQSPSSLSASVGDRVTITCRASQTITRHLNWFQQKPGKAPNLLIHGTSALQTGVPPRFSGGGSGTDFTLTINSLQPEDFGTYYCQQSHTKPWTFGPGTKVEIKRTV

SEQ ID NO: 17 - protein, 5 aa, Artificial Sequence
  source: /note=“Description of Artificial Sequence: Synthetic peptide”
SYPMN

SEQ ID NO: 18 - protein, 17 aa, Artificial Sequence
  source: /note=“Description of Artificial Sequence: Synthetic peptide”
RISRSGGRTSYADSVKG

SEQ ID NO: 19 - protein, 12 aa, Artificial Sequence
  source: /note=“Description of Artificial Sequence: Synthetic peptide”
DATRRHYNGMDV

SEQ ID NO: 20 - protein, 11 aa, Artificial Sequence
  source: /note=“Description of Artificial Sequence: Synthetic peptide”
RASRSVGKYLA

SEQ ID NO: 21 - protein, 7 aa, Artificial Sequence
  source: /note=“Description of Artificial Sequence: Synthetic peptide”
YASLRAG

SEQ ID NO: 22 - protein, 9 aa, Artificial Sequence
  source: /note=“Description of Artificial Sequence: Synthetic peptide”
QQYGSSPRT

SEQ ID NO: 23 - protein, 30 aa, Artificial Sequence
  source: /note=“Description of Artificial Sequence: Synthetic polypeptide”
EVQLLQSGGGLVQPGGSLRLSCAASGFTFS

SEQ ID NO: 24 - protein, 14 aa, Artificial Sequence
  source: /note=“Description of Artificial Sequence: Synthetic peptide”
WVRQAPGKGLEWVS

SEQ ID NO: 25 - protein, 32 aa, Artificial Sequence; identical to SEQ ID NO: 9
  source: /note=“Description of Artificial Sequence: Synthetic polypeptide”

SEQ ID NO: 26 - protein, 11 aa, Artificial Sequence
  source: /note=“Description of Artificial Sequence: Synthetic peptide”
WGKGTTVTVSS

SEQ ID NO: 27 - protein, 23 aa, Artificial Sequence
  source: /note=“Description of Artificial Sequence: Synthetic peptide”
DVVMTQSPATLSLSPGERASLSC

SEQ ID NO: 28 - protein, 15 aa, Artificial Sequence
  source: /note=“Description of Artificial Sequence: Synthetic peptide”
WYQQKPGQAPRLLFY

SEQ ID NO: 29 - protein, 32 aa, Artificial Sequence
  source: /note=“Description of Artificial Sequence: Synthetic polypeptide”
DIPSRFTASGSGTDFTLTISRLEPEDFAVYYC

SEQ ID NO: 30 - protein, 13 aa, Artificial Sequence
  source: /note=“Description of Artificial Sequence: Synthetic peptide”
FGQGTKLEMKRTV

SEQ ID NO: 31 - protein, 121 aa, Artificial Sequence
  source: /note=“Description of Artificial Sequence: Synthetic polypeptide”
EVQLLQSGGGLVQPGGSLRLSCAASGFTFSSYPMNWVRQAPGKGLEWVSRISRSGGRTSYADSVKGRFTISRDNSKNTLYLQMNSLRAEDTAVYYCARDATRRHYNGMDVWGKGTTVTVSS

SEQ ID NO: 32 - protein, 110 aa, Artificial Sequence
  source: /note=“Description of Artificial Sequence: Synthetic polypeptide”
DVVMTQSPATLSLSPGERASLSCRASRSVGKYLAWYQQKPGQAPRLLFYYASLRAGDIPSRFTASGSGTDFTLTISRLEPEDFAVYYCQQYGSSPRTFGQGTKLEMKRTV

SEQ ID NO: 33 - protein, 5 aa, Artificial Sequence
  source: /note=“Description of Artificial Sequence: Synthetic peptide”
HYPMR

SEQ ID NO: 34 - protein, 17 aa, Artificial Sequence
  source: /note=“Description of Artificial Sequence: Synthetic peptide”
SIRRSGGRTKYADSVKG

SEQ ID NO: 35 - protein, 12 aa, Artificial Sequence
  source: /note=“Description of Artificial Sequence: Synthetic peptide”
DATGRHYNGMDV

SEQ ID NO: 36 - protein, 11 aa, Artificial Sequence
  source: /note=“Description of Artificial Sequence: Synthetic peptide”
RTSQVIGTSLN

SEQ ID NO: 37 - protein, 7 aa, Artificial Sequence
  source: /note=“Description of Artificial Sequence: Synthetic peptide”
SASNLQS

SEQ ID NO: 38 - protein, 9 aa, Artificial Sequence
  source: /note=“Description of Artificial Sequence: Synthetic peptide”
QQSSRVPHT

SEQ ID NO: 39 - protein, 30 aa, Artificial Sequence
  source: /note=“Description of Artificial Sequence: Synthetic polypeptide”
QVQLVESGGGLVQPGGSLRLSCAASGFTFS

SEQ ID NO: 40 - protein, 14 aa, Artificial Sequence
  source: /note=“Description of Artificial Sequence: Synthetic peptide”
WVRQAPGKGLEWVS

SEQ ID NO: 41 - protein, 32 aa, Artificial Sequence; identical to SEQ ID NO: 9
  source: /note=“Description of Artificial Sequence: Synthetic polypeptide”

SEQ ID NO: 42 - protein, 11 aa, Artificial Sequence
  source: /note=“Description of Artificial Sequence: Synthetic peptide”
WGKGTTVTVSS

SEQ ID NO: 43 - protein, 23 aa, Artificial Sequence
  source: /note=“Description of Artificial Sequence: Synthetic peptide”
DVVMTQSPSSLSASVGDRVTITC

SEQ ID NO: 44 - protein, 15 aa, Artificial Sequence
  source: /note=“Description of Artificial Sequence: Synthetic peptide”
WYQQKPGRAPRLLIY

SEQ ID NO: 45 - protein, 32 aa, Artificial Sequence
  source: /note=“Description of Artificial Sequence: Synthetic polypeptide”
GVPSRFSGSGHGTQFTLTISSLQPEDFATYSC

SEQ ID NO: 46 - protein, 13 aa, Artificial Sequence
  source: /note=“Description of Artificial Sequence: Synthetic peptide”
FGQGTKLEMRRTV

SEQ ID NO: 47 - protein, 121 aa, Artificial Sequence
  source: /note=“Description of Artificial Sequence: Synthetic polypeptide”
QVQLVESGGGLVQPGGSLRLSCAASGFTFSHYPMRWVRQAPGKGLEWVSSIRRSGGRTKYADSVKGRFTISRDNSKNTLYLQMNSLRAEDTAVYYCARDATGRHYNGMDVWGKGTTVTVSS

SEQ ID NO: 48 - protein, 110 aa, Artificial Sequence
  source: /note=“Description of Artificial Sequence: Synthetic polypeptide”
DVVMTQSPSSLSASVGDRVTITCRTSQVIGTSLNWYQQKPGRAPRLLIYSASNLQSGVPSRFSGSGHGTQFTLTISSLQPEDFATYSCQQSSRVPHTFGQGTKLEMRRTV

SEQ ID NO: 49 - protein, 5 aa, Artificial Sequence
  source: /note=“Description of Artificial Sequence: Synthetic peptide”
RYRMG

SEQ ID NO: 50 - protein, 17 aa, Artificial Sequence
  source: /note=“Description of Artificial Sequence: Synthetic peptide”
SIARSGGRTYYADSVKG

SEQ ID NO: 51 - protein, 20 aa, Artificial Sequence
  source: /note=“Description of Artificial Sequence: Synthetic peptide”
GVRYCSSPSCSRGPRYAMDV

SEQ ID NO: 52 - protein, 11 aa, Artificial Sequence
  source: /note=“Description of Artificial Sequence: Synthetic peptide”
RASQGISSWLA

SEQ ID NO: 53 - protein, 7 aa, Artificial Sequence
  source: /note=“Description of Artificial Sequence: Synthetic peptide”
AASSLQS

SEQ ID NO: 54 - protein, 9 aa, Artificial Sequence
  source: /note=“Description of Artificial Sequence: Synthetic peptide”
QQANSFPIT

SEQ ID NO: 55 - protein, 30 aa, Artificial Sequence; identical to SEQ ID NO: 7
  source: /note=“Description of Artificial Sequence: Synthetic polypeptide”

SEQ ID NO: 56 - protein, 14 aa, Artificial Sequence
  source: /note=“Description of Artificial Sequence: Synthetic peptide”
WVRQAPGKGLEWVS

SEQ ID NO: 57 - protein, 32 aa, Artificial Sequence; identical to SEQ ID NO: 9
  source: /note=“Description of Artificial Sequence: Synthetic polypeptide”

SEQ ID NO: 58 - protein, 11 aa, Artificial Sequence
  source: /note=“Description of Artificial Sequence: Synthetic peptide”
WGKGTTVTVSS

SEQ ID NO: 59 - protein, 23 aa, Artificial Sequence
  source: /note=“Description of Artificial Sequence: Synthetic peptide”
EIVMTQSPSSVSASVGDRVTITC

SEQ ID NO: 60 - protein, 15 aa, Artificial Sequence
  source: /note=“Description of Artificial Sequence: Synthetic peptide”
WYQQKPGKAPKLLIY

SEQ ID NO: 61 - protein, 32 aa, Artificial Sequence
  source: /note=“Description of Artificial Sequence: Synthetic polypeptide”
GVPSRFSGSGSGTDFTLTISSLQPEDFATYYC

SEQ ID NO: 62 - protein, 13 aa, Artificial Sequence
  source: /note=“Description of Artificial Sequence: Synthetic peptide”
FGQGTRLEIKRTV

SEQ ID NO: 63 - protein, 129 aa, Artificial Sequence
  source: /note=“Description of Artificial Sequence: Synthetic polypeptide”
QVQLLQSGGGLVQPGGSLRLSCAASGFTFSRYRMGWVRQAPGKGLEWVSSIARSGGRTYYADSVKGRFTISRDNSKNTLYLQMNSLRAEDTAVYYCARGVRYCSSPSCSRGPRYAMDVWGKGTTVTVSS

SEQ ID NO: 64 - protein, 110 aa, Artificial Sequence
  source: /note=“Description of Artificial Sequence: Synthetic polypeptide”
EIVMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKRTV

SEQ ID NO: 65 - protein, 5 aa, Artificial Sequence
  source: /note=“Description of Artificial Sequence: Synthetic peptide”
RYRMA

SEQ ID NO: 66 - protein, 17 aa, Artificial Sequence
  source: /note=“Description of Artificial Sequence: Synthetic peptide”
NITRSGGVTRYADSVKG

SEQ ID NO: 67 - protein, 16 aa, Artificial Sequence
  source: /note=“Description of Artificial Sequence: Synthetic peptide”
DPNRVTAISSHYGMDV

SEQ ID NO: 68 - protein, 11 aa, Artificial Sequence
  source: /note=“Description of Artificial Sequence: Synthetic peptide”
RASQSISRWLA

SEQ ID NO: 69 - protein, 7 aa, Artificial Sequence
  source: /note=“Description of Artificial Sequence: Synthetic peptide”
DASNRAT

SEQ ID NO: 70 - protein, 11 aa, Artificial Sequence
  source: /note=“Description of Artificial Sequence: Synthetic peptide”
QQRSNWPPMYT

SEQ ID NO: 71 - protein, 30 aa, Artificial Sequence
  source: /note=“Description of Artificial Sequence: Synthetic polypeptide”
EVQLVQSGGGLVQPGGSLRLSCAASGFTFS

SEQ ID NO: 72 - protein, 14 aa, Artificial Sequence
  source: /note=“Description of Artificial Sequence: Synthetic peptide”
WVRQAPGKGLEWVS

SEQ ID NO: 73 - protein, 32 aa, Artificial Sequence; identical to SEQ ID NO: 9
  source: /note=“Description of Artificial Sequence: Synthetic polypeptide”

SEQ ID NO: 74 - protein, 11 aa, Artificial Sequence
  source: /note=“Description of Artificial Sequence: Synthetic peptide”
WGKGTTVTVSS

SEQ ID NO: 75 - protein, 23 aa, Artificial Sequence
  source: /note=“Description of Artificial Sequence: Synthetic peptide”
EIVLTQSPSTLSASVGDRVTISC

SEQ ID NO: 76 - protein, 15 aa, Artificial Sequence
  source: /note=“Description of Artificial Sequence: Synthetic peptide”
WYQQKPGQAPRLLIY

SEQ ID NO: 77 - protein, 32 aa, Artificial Sequence
  source: /note=“Description of Artificial Sequence: Synthetic polypeptide”
GVPARFSVSGSETDSTLTISSLEPEDFAMYYC

SEQ ID NO: 78 - protein, 13 aa, Artificial Sequence
  source: /note=“Description of Artificial Sequence: Synthetic peptide”
FGQGTKLEIKRTV

SEQ ID NO: 79 - protein, 125 aa, Artificial Sequence
  source: /note=“Description of Artificial Sequence: Synthetic polypeptide”
EVQLVQSGGGLVQPGGSLRLSCAASGFTFSRYRMAWVRQAPGKGLEWVSNITRSGGVTRYADSVKGRFTISRDNSKNTLYLQMNSLRAEDTAVYYCARDPNRVTAISSHYGMDVWGKGTTVTVSS

SEQ ID NO: 80 - protein, 112 aa, Artificial Sequence
  source: /note=“Description of Artificial Sequence: Synthetic polypeptide”
EIVLTQSPSTLSASVGDRVTISCRASQSISRWLAWYQQKPGQAPRLLIYDASNRATGVPARFSVSGSETDSTLTISSLEPEDFAMYYCQQRSNWPPMYTFGQGTKLEIKRTV

SEQ ID NO: 81 - protein, 5 aa, Artificial Sequence
  source: /note=“Description of Artificial Sequence: Synthetic peptide”
PYRMH

SEQ ID NO: 82 - protein, 17 aa, Artificial Sequence
  source: /note=“Description of Artificial Sequence: Synthetic peptide”
RINPSGGRTWYADSVKG

SEQ ID NO: 83 - protein, 12 aa, Artificial Sequence
  source: /note=“Description of Artificial Sequence: Synthetic peptide”
DATGRHYNGMDV

SEQ ID NO: 84 - protein, 11 aa, Artificial Sequence
  source: /note=“Description of Artificial Sequence: Synthetic peptide”
RASQSINKWLA

SEQ ID NO: 85 - protein, 7 aa, Artificial Sequence
  source: /note=“Description of Artificial Sequence: Synthetic peptide”
KASTLES

SEQ ID NO: 86 - protein, 9 aa, Artificial Sequence
  source: /note=“Description of Artificial Sequence: Synthetic peptide”
QQSHSAPWT

SEQ ID NO: 87 - protein, 30 aa, Artificial Sequence
  source: /note=“Description of Artificial Sequence: Synthetic polypeptide”
EVQLVESGGGLVQPGGSLRLSCAASGFTFS

SEQ ID NO: 88 - protein, 14 aa, Artificial Sequence
  source: /note=“Description of Artificial Sequence: Synthetic peptide”
WVRQAPGKGLEWVS

SEQ ID NO: 89 - protein, 32 aa, Artificial Sequence; identical to SEQ ID NO: 9
  source: /note=“Description of Artificial Sequence: Synthetic polypeptide”

SEQ ID NO: 90 - protein, 11 aa, Artificial Sequence
  source: /note=“Description of Artificial Sequence: Synthetic peptide”
WGQGTMVTVSS

SEQ ID NO: 91 - protein, 23 aa, Artificial Sequence
  source: /note=“Description of Artificial Sequence: Synthetic peptide”
DIQMTQSPSTLSASVGDRVTITC

SEQ ID NO: 92 - protein, 15 aa, Artificial Sequence
  source: /note=“Description of Artificial Sequence: Synthetic peptide”
WYQQKPGKAPKLLIY

SEQ ID NO: 93 - protein, 32 aa, Artificial Sequence
  source: /note=“Description of Artificial Sequence: Synthetic polypeptide”
GVPSRFSGSGSGTDFTLTINSLQPEDFATYYC

SEQ ID NO: 94 - protein, 13 aa, Artificial Sequence
  source: /note=“Description of Artificial Sequence: Synthetic peptide”
FGQGTKVEIERTV

SEQ ID NO: 95 - protein, 121 aa, Artificial Sequence
  source: /note=“Description of Artificial Sequence: Synthetic polypeptide”
EVQLVESGGGLVQPGGSLRLSCAASGFTFSPYRMHWVRQAPGKGLEWVSRINPSGGRTWYADSVKGRFTISRDNSKNTLYLQMNSLRAEDTAVYYCARDATGRHYNGMDVWGQGTMVTVSS

SEQ ID NO: 96 - protein, 110 aa, Artificial Sequence
  source: /note=“Description of Artificial Sequence: Synthetic polypeptide”
DIQMTQSPSTLSASVGDRVTITCRASQSINKWLAWYQQKPGKAPKLLIYKASTLESGVPSRFSGSGSGTDFTLTINSLQPEDFATYYCQQSHSAPWTFGQGTKVEIERTV

SEQ ID NO: 97 - protein, 5 aa, Artificial Sequence
  source: /note=“Description of Artificial Sequence: Synthetic peptide”
SYPMN

SEQ ID NO: 98 - protein, 17 aa, Artificial Sequence
  source: /note=“Description of Artificial Sequence: Synthetic peptide”
RISRSGGRTSYADSVKG

SEQ ID NO: 99 - protein, 12 aa, Artificial Sequence
  source: /note=“Description of Artificial Sequence: Synthetic peptide”
DATRRHYNGMDV

SEQ ID NO: 100 - protein, 11 aa, Artificial Sequence
  source: /note=“Description of Artificial Sequence: Synthetic peptide”
RASRSVGKYLA

SEQ ID NO: 101 - protein, 7 aa, Artificial Sequence
  source: /note=“Description of Artificial Sequence: Synthetic peptide”
YASLRAG

SEQ ID NO: 102 - protein, 9 aa, Artificial Sequence
  source: /note=“Description of Artificial Sequence: Synthetic peptide”
QQYGSSPRT

SEQ ID NO: 103 - protein, 30 aa, Artificial Sequence
  source: /note=“Description of Artificial Sequence: Synthetic polypeptide”
EVQLEESGGGLVQPGGSLRLSCAASGFTFS

SEQ ID NO: 104 - protein, 14 aa, Artificial Sequence
  source: /note=“Description of Artificial Sequence: Synthetic peptide”
WVRQAPGKGLEWVS

SEQ ID NO: 105 - protein, 32 aa, Artificial Sequence; identical to SEQ ID NO: 9
  source: /note=“Description of Artificial Sequence: Synthetic polypeptide”

SEQ ID NO: 106 - protein, 11 aa, Artificial Sequence
  source: /note=“Description of Artificial Sequence: Synthetic peptide”
WGKGTTVTVSS

SEQ ID NO: 107 - protein, 23 aa, Artificial Sequence
  source: /note=“Description of Artificial Sequence: Synthetic peptide”
ETTLTQSPATLSLSPGERASLSC

SEQ ID NO: 108 - protein, 15 aa, Artificial Sequence
  source: /note=“Description of Artificial Sequence: Synthetic peptide”
WYQQKPGQAPRLLFY

SEQ ID NO: 109 - protein, 32 aa, Artificial Sequence; identical to SEQ ID NO: 29
  source: /note=“Description of Artificial Sequence: Synthetic polypeptide”

SEQ ID NO: 110 - protein, 13 aa, Artificial Sequence
  source: /note=“Description of Artificial Sequence: Synthetic peptide”
FGQGTKLEMKRTV

SEQ ID NO: 111 - protein, 121 aa, Artificial Sequence
  source: /note=“Description of Artificial Sequence: Synthetic polypeptide”
EVQLEESGGGLVQPGGSLRLSCAASGFTFSSYPMNWVRQAPGKGLEWVSRISRSGGRTSYADSVKGRFTISRDNSKNTLYLQMNSLRAEDTAVYYCARDATRRHYNGMDVWGKGTTVTVSS

SEQ ID NO: 112 - protein, 110 aa, Artificial Sequence
  source: /note=“Description of Artificial Sequence: Synthetic polypeptide”
ETTLTQSPATLSLSPGERASLSCRASRSVGKYLAWYQQKPGQAPRLLFYYASLRAGDIPSRFTASGSGTDFTLTISRLEPEDFAVYYCQQYGSSPRTFGQGTKLEMKRTV

SEQ ID NO: 113 - protein, 5 aa, Artificial Sequence
  source: /note=“Description of Artificial Sequence: Synthetic peptide”
SYAMS

SEQ ID NO: 114 - protein, 17 aa, Artificial Sequence
  source: /note=“Description of Artificial Sequence: Synthetic peptide”
AISGSGGSTYYADSVKG

SEQ ID NO: 115 - protein, 15 aa, Artificial Sequence
  source: /note=“Description of Artificial Sequence: Synthetic peptide”
PYNSAWESYYYGMDV

SEQ ID NO: 116 - protein, 11 aa, Artificial Sequence
  source: /note=“Description of Artificial Sequence: Synthetic peptide”
RASQGISSRLA

SEQ ID NO: 117 - protein, 7 aa, Artificial Sequence
  source: /note=“Description of Artificial Sequence: Synthetic peptide”
AASSLQS

SEQ ID NO: 118 - protein, 9 aa, Artificial Sequence
  source: /note=“Description of Artificial Sequence: Synthetic peptide”
QQRHSYPIT

SEQ ID NO: 119 - protein, 30 aa, Artificial Sequence
  source: /note=“Description of Artificial Sequence: Synthetic polypeptide”
EVQLQESGGGLVQPGGSLRLSCAASGFTFS

SEQ ID NO: 120 - protein, 14 aa, Artificial Sequence
  source: /note=“Description of Artificial Sequence: Synthetic peptide”
WVRQAPGKGLEWVS

SEQ ID NO: 121 - protein, 32 aa, Artificial Sequence; identical to SEQ ID NO: 9
  source: /note=“Description of Artificial Sequence: Synthetic polypeptide”

SEQ ID NO: 122 - protein, 11 aa, Artificial Sequence
  source: /note=“Description of Artificial Sequence: Synthetic peptide”
WGKGTTVTVSS

SEQ ID NO: 123 - protein, 23 aa, Artificial Sequence
  source: /note=“Description of Artificial Sequence: Synthetic peptide”
DIQMTQSPSSVSASVGDRVTITC

SEQ ID NO: 124 - protein, 15 aa, Artificial Sequence
  source: /note=“Description of Artificial Sequence: Synthetic peptide”
WYQQKPGKAPKLLIY

SEQ ID NO: 125 - protein, 32 aa, Artificial Sequence; identical to SEQ ID NO: 61
  source: /note=“Description of Artificial Sequence: Synthetic polypeptide”

SEQ ID NO: 126 - protein, 13 aa, Artificial Sequence
  source: /note=“Description of Artificial Sequence: Synthetic peptide”
FGQGTRLEIKRTV

SEQ ID NO: 127 - protein, 124 aa, Artificial Sequence
  source: /note=“Description of Artificial Sequence: Synthetic polypeptide”
EVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCARPYNSAWESYYYGMDVWGKGTTVTVSS

SEQ ID NO: 128 - protein, 110 aa, Artificial Sequence
  source: /note=“Description of Artificial Sequence: Synthetic polypeptide”
DIQMTQSPSSVSASVGDRVTITCRASQGISSRLAWYQQKPGKAPKLLIYAASSLQSGVPSRFSGSGSGTDFTLTISSLQPEDFATYYCQQRHSYPITFGQGTRLEIKRTV

SEQ ID NO: 129 - protein, 5 aa, Artificial Sequence
  source: /note=“Description of Artificial Sequence: Synthetic peptide”
RYDMS

SEQ ID NO: 130 - protein, 17 aa, Artificial Sequence
  source: /note=“Description of Artificial Sequence: Synthetic peptide”
RIRYSGGRTGYADSVKG

SEQ ID NO: 131 - protein, 20 aa, Artificial Sequence
  source: /note=“Description of Artificial Sequence: Synthetic peptide”
GVRYCSSPSCSRGPRYAMDV

SEQ ID NO: 132 - protein, 11 aa, Artificial Sequence
  source: /note=“Description of Artificial Sequence: Synthetic peptide”
RASQSVRGYLA

SEQ ID NO: 133 - protein, 7 aa, Artificial Sequence
  source: /note=“Description of Artificial Sequence: Synthetic peptide”
DTFKRAT

SEQ ID NO: 134 - protein, 9 aa, Artificial Sequence
  source: /note=“Description of Artificial Sequence: Synthetic peptide”
QQYFASPWT

SEQ ID NO: 135 - protein, 30 aa, Artificial Sequence; identical to SEQ ID NO: 87
  source: /note=“Description of Artificial Sequence: Synthetic polypeptide”

SEQ ID NO: 136 - protein, 14 aa, Artificial Sequence
  source: /note=“Description of Artificial Sequence: Synthetic peptide”
WVRQAPGKGLEWVS

SEQ ID NO: 137 - protein, 32 aa, Artificial Sequence; identical to SEQ ID NO: 9
  source: /note=“Description of Artificial Sequence: Synthetic polypeptide”

SEQ ID NO: 138 - protein, 11 aa, Artificial Sequence
  source: /note=“Description of Artificial Sequence: Synthetic peptide”
WGKGTTVTVSS

SEQ ID NO: 139 - protein, 23 aa, Artificial Sequence
  source: /note=“Description of Artificial Sequence: Synthetic peptide”
DVVMTQSPATLSLSPGEGATLSC

SEQ ID NO: 140 - protein, 15 aa, Artificial Sequence
  source: /note=“Description of Artificial Sequence: Synthetic peptide”
WYQQKPGQAPRLLIY

SEQ ID NO: 141 - protein, 32 aa, Artificial Sequence
  source: /note=“Description of Artificial Sequence: Synthetic polypeptide”
GIPARFSGSGSGADFTLTISSLEPEDSAVYYC

SEQ ID NO: 142 - protein, 13 aa, Artificial Sequence
  source: /note=“Description of Artificial Sequence: Synthetic peptide”
FGQGTKVEIKRTV

SEQ ID NO: 143 - protein, 129 aa, Artificial Sequence
  source: /note=“Description of Artificial Sequence: Synthetic polypeptide”
EVQLVESGGGLVQPGGSLRLSCAASGFTFSRYDMSWVRQAPGKGLEWVSRIRYSGGRTGYADSVKGRFTISRDNSKNTLYLQMNSLRAEDTAVYYCARGVRYCSSPSCSRGPRYAMDVWGKGTTVTVSS

SEQ ID NO: 144 - protein, 110 aa, Artificial Sequence
  source: /note=“Description of Artificial Sequence: Synthetic polypeptide”
DVVMTQSPATLSLSPGEGATLSCRASQSVRGYLAWYQQKPGQAPRLLIYDTFKRATGIPARFSGSGSGADFTLTISSLEPEDSAVYYCQQYFASPWTFGQGTKVEIKRTV

SEQ ID NO: 145 - protein, 5 aa, Artificial Sequence
  source: /note=“Description of Artificial Sequence: Synthetic peptide”
RYRMY

SEQ ID NO: 146 - protein, 17 aa, Artificial Sequence
  source: /note=“Description of Artificial Sequence: Synthetic peptide”
TISRSGGRTVYADSVKG

SEQ ID NO: 147 - protein, 12 aa, Artificial Sequence
  source: /note=“Description of Artificial Sequence: Synthetic peptide”
DATGRHYNGMDV

SEQ ID NO: 148 - protein, 11 aa, Artificial Sequence
  source: /note=“Description of Artificial Sequence: Synthetic peptide”
RASQSVSSNVA

SEQ ID NO: 149 - protein, 7 aa, Artificial Sequence
  source: /note=“Description of Artificial Sequence: Synthetic peptide”
GSGTRAT

SEQ ID NO: 150 - protein, 8 aa, Artificial Sequence
  source: /note=“Description of Artificial Sequence: Synthetic peptide”
QQYNDWPS

SEQ ID NO: 151 - protein, 30 aa, Artificial Sequence
  source: /note=“Description of Artificial Sequence: Synthetic polypeptide”
EVQLLESGGGLVQPGGSLRLSCAASGFTFS

SEQ ID NO: 152 - protein, 14 aa, Artificial Sequence
  source: /note=“Description of Artificial Sequence: Synthetic peptide”
WVRQAPGKGLEWVS

SEQ ID NO: 153 - protein, 32 aa, Artificial Sequence; identical to SEQ ID NO: 9
  source: /note=“Description of Artificial Sequence: Synthetic polypeptide”

SEQ ID NO: 154 - protein, 9 aa, Artificial Sequence
  source: /note=“Description of Artificial Sequence: Synthetic peptide”
WGQGTLVTV

SEQ ID NO: 155 - protein, 23 aa, Artificial Sequence
  source: /note=“Description of Artificial Sequence: Synthetic peptide”
ETTLTQSPATLSVSPGERATLSC

SEQ ID NO: 156 - protein, 15 aa, Artificial Sequence
  source: /note=“Description of Artificial Sequence: Synthetic peptide”
WYQQKPGQAPRLLMF

SEQ ID NO: 157 - protein, 32 aa, Artificial Sequence
  source: /note=“Description of Artificial Sequence: Synthetic polypeptide”
GIPARFSGSGSGTEFTLTISSLQSEDFAAYYC

SEQ ID NO: 158 - protein, 13 aa, Artificial Sequence
  source: /note=“Description of Artificial Sequence: Synthetic peptide”
FGQGTRVEIKGTV

SEQ ID NO: 159 - protein, 119 aa, Artificial Sequence
  source: /note=“Description of Artificial Sequence: Synthetic polypeptide”
EVQLLESGGGLVQPGGSLRLSCAASGFTFSRYRMYWVRQAPGKGLEWVSTISRSGGRTVYADSVKGRFTISRDNSKNTLYLQMNSLRAEDTAVYYCARDATGRHYNGMDVWGQGTLVTV

SEQ ID NO: 160 - protein, 109 aa, Artificial Sequence
  source: /note=“Description of Artificial Sequence: Synthetic polypeptide”
ETTLTQSPATLSVSPGERATLSCRASQSVSSNVAWYQQKPGQAPRLLMFGSGTRATGIPARFSGSGSGTEFTLTISSLQSEDFAAYYCQQYNDWPSFGQGTRVEIKGTV

SEQ ID NO: 161 - protein, 5 aa, Artificial Sequence
  source: /note=“Description of Artificial Sequence: Synthetic peptide”
RYRMY

SEQ ID NO: 162 - protein, 17 aa, Artificial Sequence
  source: /note=“Description of Artificial Sequence: Synthetic peptide”
SISSSGGRTKYADSVKG

SEQ ID NO: 163 - protein, 20 aa, Artificial Sequence
  source: /note=“Description of Artificial Sequence: Synthetic peptide”
GVRYCSSPSCSRGPRYAMDV

SEQ ID NO: 164 - protein, 11 aa, Artificial Sequence
  source: /note=“Description of Artificial Sequence: Synthetic peptide”
RASYVIRNDLS

SEQ ID NO: 165 - protein, 7 aa, Artificial Sequence
  source: /note=“Description of Artificial Sequence: Synthetic peptide”
GTSSLHN

SEQ ID NO: 166 - protein, 9 aa, Artificial Sequence
  source: /note=“Description of Artificial Sequence: Synthetic peptide”
LQDDKYPLT

SEQ ID NO: 167 - protein, 30 aa, Artificial Sequence; identical to SEQ ID NO: 87
  source: /note=“Description of Artificial Sequence: Synthetic polypeptide”

SEQ ID NO: 168 - protein, 14 aa, Artificial Sequence
  source: /note=“Description of Artificial Sequence: Synthetic peptide”
WVRQAPGKGLEWVS

SEQ ID NO: 169 - protein, 32 aa, Artificial Sequence; identical to SEQ ID NO: 9
  source: /note=“Description of Artificial Sequence: Synthetic polypeptide”

SEQ ID NO: 170 - protein, 11 aa, Artificial Sequence
  source: /note=“Description of Artificial Sequence: Synthetic peptide”
WGKGTTVTVSS

SEQ ID NO: 171 - protein, 23 aa, Artificial Sequence
  source: /note=“Description of Artificial Sequence: Synthetic peptide”
DIQMTQSPSSLSASVGDRVTITC

SEQ ID NO: 172 - protein, 15 aa, Artificial Sequence
  source: /note=“Description of Artificial Sequence: Synthetic peptide”
WYQQKPGKAPKLLIY

SEQ ID NO: 173 - protein, 32 aa, Artificial Sequence
  source: /note=“Description of Artificial Sequence: Synthetic polypeptide”
GVPSRFSGSGYGTYFTLTISSLQPEDFGTYYC

SEQ ID NO: 174 - protein, 13 aa, Artificial Sequence
  source: /note=“Description of Artificial Sequence: Synthetic peptide”
FGGGTKVEIKRTV

SEQ ID NO: 175 - protein, 129 aa, Artificial Sequence
  source: /note=“Description of Artificial Sequence: Synthetic polypeptide”
EVQLVESGGGLVQPGGSLRLSCAASGFTFSRYRMYWVRQAPGKGLEWVSSISSSGGRTKYADSVKGRFTISRDNSKNTLYLQMNSLRAEDTAVYYCARGVRYCSSPSCSRGPRYAMDVWGKGTTVTVSS

SEQ ID NO: 176 - protein, 110 aa, Artificial Sequence
  source: /note=“Description of Artificial Sequence: Synthetic polypeptide”
DIQMTQSPSSLSASVGDRVTITCRASYVIRNDLSWYQQKPGKAPKLLIYGTSSLHNGVPSRFSGSGYGTYFTLTISSLQPEDFGTYYCLQDDKYPLTFGGGTKVEIKRTV

SEQ ID NO: 177 - protein, 5 aa, Artificial Sequence
  source: /note=“Description of Artificial Sequence: Synthetic peptide”
KYKMS

SEQ ID NO: 178 - protein, 17 aa, Artificial Sequence
  source: /note=“Description of Artificial Sequence: Synthetic peptide”
TIAPSGGGTRYADSVKG

SEQ ID NO: 179 - protein, 7 aa, Artificial Sequence
  source: /note=“Description of Artificial Sequence: Synthetic peptide”
GGHFSNP

SEQ ID NO: 180 - protein, 16 aa, Artificial Sequence
  source: /note=“Description of Artificial Sequence: Synthetic peptide”
RSSQSLVHTDGDTYLN

SEQ ID NO: 181 - protein, 7 aa, Artificial Sequence
  source: /note=“Description of Artificial Sequence: Synthetic peptide”
KVSKRDS

SEQ ID NO: 182 - protein, 9 aa, Artificial Sequence
  source: /note=“Description of Artificial Sequence: Synthetic peptide”
MQGIKRPYT

SEQ ID NO: 183 - protein, 30 aa, Artificial Sequence; identical to SEQ ID NO: 87
  source: /note=“Description of Artificial Sequence: Synthetic polypeptide”

SEQ ID NO: 184 - protein, 14 aa, Artificial Sequence
  source: /note=“Description of Artificial Sequence: Synthetic peptide”
WVRQAPGKGLEWVS

SEQ ID NO: 185 - protein, 32 aa, Artificial Sequence; identical to SEQ ID NO: 9
  source: /note=“Description of Artificial Sequence: Synthetic polypeptide”

SEQ ID NO: 186 - protein, 11 aa, Artificial Sequence
  source: /note=“Description of Artificial Sequence: Synthetic peptide”
WGQGTLVTVSS

SEQ ID NO: 187 - protein, 23 aa, Artificial Sequence
  source: /note=“Description of Artificial Sequence: Synthetic peptide”
DVVMTQSPLSLPVTLGQPASISC

SEQ ID NO: 188 - protein, 15 aa, Artificial Sequence
  source: /note=“Description of Artificial Sequence: Synthetic peptide”
WYQQRPGQSPRRLIY

SEQ ID NO: 189 - protein, 32 aa, Artificial Sequence
  source: /note=“Description of Artificial Sequence: Synthetic polypeptide”
GVPDRFSGSGSGTDFTLKISRVEAEDVGVYYC

SEQ ID NO: 190 - protein, 13 aa, Artificial Sequence
  source: /note=“Description of Artificial Sequence: Synthetic peptide”
LGQGTKLEIKRTV

SEQ ID NO: 191 - protein, 116 aa, Artificial Sequence
  source: /note=“Description of Artificial Sequence: Synthetic polypeptide”
EVQLVESGGGLVQPGGSLRLSCAASGFTFSKYKMSWVRQAPGKGLEWVSTIAPSGGGTRYADSVKGRFTISRDNSKNTLYLQMNSLRAEDTAVYYCARGGHFSNPWGQGTLVTVSS

SEQ ID NO: 192 - protein, 115 aa, Artificial Sequence
  source: /note=“Description of Artificial Sequence: Synthetic polypeptide”
DVVMTQSPLSLPVTLGQPASISCRSSQSLVHTDGDTYLNWYQQRPGQSPRRLIYKVSKRDSGVPDRFSGSGSGTDFTLKISRVEAEDVGVYYCMQGIKRPYTLGQGTKLEIKRTV

SEQ ID NO: 193 - protein, 5 aa, Artificial Sequence
  source: /note=“Description of Artificial Sequence: Synthetic peptide”
PYRMH

SEQ ID NO: 194 - protein, 17 aa, Artificial Sequence
  source: /note=“Description of Artificial Sequence: Synthetic peptide”
SINRSGGRTNYADSVKG

SEQ ID NO: 195 - protein, 9 aa, Artificial Sequence
  source: /note=“Description of Artificial Sequence: Synthetic peptide”
GRGIGTFRN

SEQ ID NO: 196 - protein, 11 aa, Artificial Sequence
  source: /note=“Description of Artificial Sequence: Synthetic peptide”
RASQSVSTYLA

SEQ ID NO: 197 - protein, 7 aa, Artificial Sequence
  source: /note=“Description of Artificial Sequence: Synthetic peptide”
DASNRAT

SEQ ID NO: 198 - protein, 9 aa, Artificial Sequence
  source: /note=“Description of Artificial Sequence: Synthetic peptide”
QQRNNWPPT

SEQ ID NO: 199 - protein, 30 aa, Artificial Sequence; identical to SEQ ID NO: 87
  source: /note=“Description of Artificial Sequence: Synthetic polypeptide”

SEQ ID NO: 200 - protein, 14 aa, Artificial Sequence
  source: /note=“Description of Artificial Sequence: Synthetic peptide”
WVRQAPGKGLEWVS

SEQ ID NO: 201 - protein, 32 aa, Artificial Sequence
  source: /note=“Description of Artificial Sequence: Synthetic polypeptide”
RFTISRDNSKNTLYLQMNSLRAEDTAVYYCAT

SEQ ID NO: 202 - protein, 11 aa, Artificial Sequence
  source: /note=“Description of Artificial Sequence: Synthetic peptide”
WGQGTLVTVSS

SEQ ID NO: 203 - protein, 23 aa, Artificial Sequence
  source: /note=“Description of Artificial Sequence: Synthetic peptide”
DIALTQSPATLSLSPGERATLSC

SEQ ID NO: 204 - protein, 15 aa, Artificial Sequence
  source: /note=“Description of Artificial Sequence: Synthetic peptide”
WYQQKPGQAPRLLIS

SEQ ID NO: 205 - protein, 32 aa, Artificial Sequence
  source: /note=“Description of Artificial Sequence: Synthetic polypeptide”
GIPARFSGSGSGTDFTLTISSLEPEDFAVYYC

SEQ ID NO: 206 - protein, 13 aa, Artificial Sequence
  source: /note=“Description of Artificial Sequence: Synthetic peptide”
FGQGTKVEIKRTV

SEQ ID NO: 207 - protein, 118 aa, Artificial Sequence
  source: /note=“Description of Artificial Sequence: Synthetic polypeptide”
EVQLVESGGGLVQPGGSLRLSCAASGFTFSPYRMHWVRQAPGKGLEWVSSINRSGGRTNYADSVKGRFTISRDNSKNTLYLQMNSLRAEDTAVYYCATGRGIGTFRNWGQGTLVTVSS

SEQ ID NO: 208 - protein, 110 aa, Artificial Sequence
  source: /note=“Description of Artificial Sequence: Synthetic polypeptide”
DIALTQSPATLSLSPGERATLSCRASQSVSTYLAWYQQKPGQAPRLLISDASNRATGIPARFSGSGSGTDFTLTISSLEPEDFAVYYCQQRNNWPPTFGQGTKVEIKRTV

SEQ ID NO: 209 - protein, 5 aa, Artificial Sequence
  source: /note=“Description of Artificial Sequence: Synthetic peptide”
SYAMS

SEQ ID NO: 210 - protein, 17 aa, Artificial Sequence
  source: /note=“Description of Artificial Sequence: Synthetic peptide”
AISGSGGSTYYADSVKG

SEQ ID NO: 211 - protein, 9 aa, Artificial Sequence
  source: /note=“Description of Artificial Sequence: Synthetic peptide”
DTIPGYMDV

SEQ ID NO: 212 - protein, 11 aa, Artificial Sequence
  source: /note=“Description of Artificial Sequence: Synthetic peptide”
RASQSISNYLS

SEQ ID NO: 213 - protein, 7 aa, Artificial Sequence
  source: /note=“Description of Artificial Sequence: Synthetic peptide”
AASSLQS

SEQ ID NO: 214 - protein, 9 aa, Artificial Sequence
  source: /note=“Description of Artificial Sequence: Synthetic peptide”
QQSYSSPYT

SEQ ID NO: 215 - protein, 30 aa, Artificial Sequence; identical to SEQ ID NO: 87
  source: /note=“Description of Artificial Sequence: Synthetic polypeptide”

SEQ ID NO: 216 - protein, 14 aa, Artificial Sequence
  source: /note=“Description of Artificial Sequence: Synthetic peptide”
WVRQAPGKGLEWVS

SEQ ID NO: 217 - protein, 32 aa, Artificial Sequence; identical to SEQ ID NO: 9
  source: /note=“Description of Artificial Sequence: Synthetic polypeptide”

SEQ ID NO: 218 - protein, 11 aa, Artificial Sequence
  source: /note=“Description of Artificial Sequence: Synthetic peptide”
WGKGTTVTVSS

SEQ ID NO: 219 - protein, 23 aa, Artificial Sequence
  source: /note=“Description of Artificial Sequence: Synthetic peptide”
DIMLTQSPSSLSGSVGDSVTFTC

SEQ ID NO: 220 - protein, 15 aa, Artificial Sequence
  source: /note=“Description of Artificial Sequence: Synthetic peptide”
WYQQKSGKAPQLLIY

SEQ ID NO: 221 - protein, 32 aa, Artificial Sequence; identical to SEQ ID NO: 61
  source: /note=“Description of Artificial Sequence: Synthetic polypeptide”

SEQ ID NO: 222 - protein, 13 aa, Artificial Sequence
  source: /note=“Description of Artificial Sequence: Synthetic peptide”
FGQGTKLEIKRTV

SEQ ID NO: 223 - protein, 118 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARDTIPGYMDVWGKGTTVTVSS

SEQ ID NO: 224 - protein, 110 aa, Artificial Sequence
  source: /note=“Description of Artificial Sequence: Synthetic polypeptide”
DIMLTQSPSSLSGSVGDSVTFTCRASQSISNYLSWYQQKSGKAPQLLIYAASSLQSGVPSRFSGSGSGTDFTLTISSLQPEDFATYYCQQSYSSPYTFGQGTKLEIKRTV

SEQ ID NO: 225 - protein, 5 aa, Artificial Sequence
  source: /note=“Description of Artificial Sequence: Synthetic peptide”
RYRMA

SEQ ID NO: 226 - protein, 17 aa, Artificial Sequence
  source: /note=“Description of Artificial Sequence: Synthetic peptide”
AIARSGGRTWYADSVKG

SEQ ID NO: 227 - protein, 13 aa, Artificial Sequence
  source: /note=“Description of Artificial Sequence: Synthetic peptide”
GGGAKWLYNWFDS

SEQ ID NO: 228 - protein, 12 aa, Artificial Sequence
  source: /note=“Description of Artificial Sequence: Synthetic peptide”
RASQSVSNTYLA

SEQ ID NO: 229 - protein, 7 aa, Artificial Sequence
  source: /note=“Description of Artificial Sequence: Synthetic peptide”
GASIRAP

SEQ ID NO: 230 - protein, 9 aa, Artificial Sequence
  source: /note=“Description of Artificial Sequence: Synthetic peptide”
QQYARSRIA

SEQ ID NO: 231 - protein, 30 aa, Artificial Sequence; identical to SEQ ID NO: 87
  source: /note=“Description of Artificial Sequence: Synthetic polypeptide”

SEQ ID NO: 232 - protein, 14 aa, Artificial Sequence
  source: /note=“Description of Artificial Sequence: Synthetic peptide”
WVRQAPGKGLEWVS

SEQ ID NO: 233 - protein, 32 aa, Artificial Sequence; identical to SEQ ID NO: 9
  source: /note=“Description of Artificial Sequence: Synthetic polypeptide”

SEQ ID NO: 234 - protein, 9 aa, Artificial Sequence
  source: /note=“Description of Artificial Sequence: Synthetic peptide”
WGQGTLVTV

SEQ ID NO: 235 - protein, 23 aa, Artificial Sequence
  source: /note=“Description of Artificial Sequence: Synthetic peptide”
EIVLTQSPGTLSLSAGERATLSC

SEQ ID NO: 236 - protein, 15 aa, Artificial Sequence
  source: /note=“Description of Artificial Sequence: Synthetic peptide”
WYQQKPGQAPRLLIY

SEQ ID NO: 237 - protein, 32 aa, Artificial Sequence
  source: /note=“Description of Artificial Sequence: Synthetic polypeptide”
GIPDRFSGSGSGTDFTLTVNRLEPEDSAVYYC

SEQ ID NO: 238 - protein, 13 aa, Artificial Sequence
  source: /note=“Description of Artificial Sequence: Synthetic peptide”
FGQGTRLEIRRTV

SEQ ID NO: 239 - protein, 120 aa, Artificial Sequence
  source: /note=“Description of Artificial Sequence: Synthetic polypeptide”
EVQLVESGGGLVQPGGSLRLSCAASGFTFSRYRMAWVRQAPGKGLEWVSAIARSGGRTWYADSVKGRFTISRDNSKNTLYLQMNSLRAEDTAVYYCARGGGAKWLYNWFDSWGQGTLVTV

SEQ ID NO: 240 - protein, 111 aa, Artificial Sequence
  source: /note=“Description of Artificial Sequence: Synthetic polypeptide”
EIVLTQSPGTLSLSAGERATLSCRASQSVSNTYLAWYQQKPGQAPRLLIYGASIRAPGIPDRFSGSGSGTDFTLTVNRLEPEDSAVYYCQQYARSRIAFGQGTRLEIRRTV

SEQ ID NO: 241 - protein, 5 aa, Artificial Sequence
  source: /note=“Description of Artificial Sequence: Synthetic peptide”
HYWMG

SEQ ID NO: 242 - protein, 17 aa, Artificial Sequence
  source: /note=“Description of Artificial Sequence: Synthetic peptide”
GIGASGGWTGYADSVKG

SEQ ID NO: 243 - protein, 8 aa, Artificial Sequence
  source: /note=“Description of Artificial Sequence: Synthetic peptide”
TSGAYFDY

SEQ ID NO: 244 - protein, 12 aa, Artificial Sequence
  source: /note=“Description of Artificial Sequence: Synthetic peptide”
RASQSVSSDYLA

SEQ ID NO: 245 - protein, 7 aa, Artificial Sequence
  source: /note=“Description of Artificial Sequence: Synthetic peptide”
GASSRAT

SEQ ID NO: 246 - protein, 9 aa, Artificial Sequence
  source: /note=“Description of Artificial Sequence: Synthetic peptide”
QQYGSTPLT

SEQ ID NO: 247 - protein, 30 aa, Artificial Sequence; identical to SEQ ID NO: 87
  source: /note=“Description of Artificial Sequence: Synthetic polypeptide”

SEQ ID NO: 248 - protein, 14 aa, Artificial Sequence
  source: /note=“Description of Artificial Sequence: Synthetic peptide”
WVRQAPGKGLEWVS

SEQ ID NO: 249 - protein, 32 aa, Artificial Sequence; identical to SEQ ID NO: 9
  source: /note=“Description of Artificial Sequence: Synthetic polypeptide”

SEQ ID NO: 250 - protein, 11 aa, Artificial Sequence
  source: /note=“Description of Artificial Sequence: Synthetic peptide”
WGQGTLVTVSS

SEQ ID NO: 251 - protein, 23 aa, Artificial Sequence
  source: /note=“Description of Artificial Sequence: Synthetic peptide”
EIVLTQSPGTLSLSPGQRATLSC

SEQ ID NO: 252 - protein, 15 aa, Artificial Sequence
  source: /note=“Description of Artificial Sequence: Synthetic peptide”
WYQQKPGQAPRLLMY

SEQ ID NO: 253 - protein, 32 aa, Artificial Sequence
  source: /note=“Description of Artificial Sequence: Synthetic polypeptide”
GIPDRFSGSGSGTDFTLTISRLEPEDFAVYYC

SEQ ID NO: 254 - protein, 13 aa, Artificial Sequence
  source: /note=“Description of Artificial Sequence: Synthetic peptide”
FGGGTTVEIRRTV

SEQ ID NO: 255 - protein, 117 aa, Artificial Sequence
  source: /note=“Description of Artificial Sequence: Synthetic polypeptide”
EVQLVESGGGLVQPGGSLRLSCAASGFTFSHYWMGWVRQAPGKGLEWVSGIGASGGWTGYADSVKGRFTISRDNSKNTLYLQMNSLRAEDTAVYYCARTSGAYFDYWGQGTLVTVSS

SEQ ID NO: 256 - protein, 111 aa, Artificial Sequence
  source: /note=“Description of Artificial Sequence: Synthetic polypeptide”
EIVLTQSPGTLSLSPGQRATLSCRASQSVSSDYLAWYQQKPGQAPRLLMYGASSRATGIPDRFSGSGSGTDFTLTISRLEPEDFAVYYCQQYGSTPLTFGGGTTVEIRRTV

SEQ ID NO: 257 - protein, 5 aa, Artificial Sequence
  source: /note=“Description of Artificial Sequence: Synthetic peptide”
NYPMT

SEQ ID NO: 258 - protein, 17 aa, Artificial Sequence
  source: /note=“Description of Artificial Sequence: Synthetic peptide”
TIRGSGGDTWYADSVKG

SEQ ID NO: 259 - protein, 6 aa, Artificial Sequence
  source: /note=“Description of Artificial Sequence: Synthetic peptide”
WVGRDA

SEQ ID NO: 260 - protein, 16 aa, Artificial Sequence
  source: /note=“Description of Artificial Sequence: Synthetic peptide”
RSSQSLVYSDGNTYLN

SEQ ID NO: 261 - protein, 7 aa, Artificial Sequence
  source: /note=“Description of Artificial Sequence: Synthetic peptide”
KVSNRDS

SEQ ID NO: 262 - protein, 9 aa, Artificial Sequence
  source: /note=“Description of Artificial Sequence: Synthetic peptide”
MQGTHWPPT

SEQ ID NO: 263 - protein, 30 aa, Artificial Sequence; identical to SEQ ID NO: 87
  source: /note=“Description of Artificial Sequence: Synthetic polypeptide”

SEQ ID NO: 264 - protein, 14 aa, Artificial Sequence
  source: /note=“Description of Artificial Sequence: Synthetic peptide”
WVRQAPGKGLEWVS

SEQ ID NO: 265 - protein, 32 aa, Artificial Sequence
  source: /note=“Description of Artificial Sequence: Synthetic polypeptide”
RFTISRDNSKNTLYLQMNSLRAEDTAVYYCAK

SEQ ID NO: 266 - protein, 11 aa, Artificial Sequence
  source: /note=“Description of Artificial Sequence: Synthetic peptide”
WGQGTLVTVSS

SEQ ID NO: 267 - protein, 23 aa, Artificial Sequence
  source: /note=“Description of Artificial Sequence: Synthetic peptide”
DIVLTQSPLSLPVTLGQPASISC

SEQ ID NO: 268 - protein, 15 aa, Artificial Sequence
  source: /note=“Description of Artificial Sequence: Synthetic peptide”
WFRQRPGQSPRRLIY

SEQ ID NO: 269 - protein, 32 aa, Artificial Sequence
  source: /note=“Description of Artificial Sequence: Synthetic polypeptide”
GVPDRFSGSGSGTDFTLRISRVEAEDVGVYYC

SEQ ID NO: 270 - protein, 13 aa, Artificial Sequence
  source: /note=“Description of Artificial Sequence: Synthetic peptide”
FGQGTKLEIKRTV

SEQ ID NO: 271 - protein, 115 aa, Artificial Sequence
  source: /note=“Description of Artificial Sequence: Synthetic polypeptide”
EVQLVESGGGLVQPGGSLRLSCAASGFTFSNYPMTWVRQAPGKGLEWVSTIRGSGGDTWYADSVKGRFTISRDNSKNTLYLQMNSLRAEDTAVYYCAKWVGRDAWGQGTLVTVSS

SEQ ID NO: 272 - protein, 115 aa, Artificial Sequence
  source: /note=“Description of Artificial Sequence: Synthetic polypeptide”
DIVLTQSPLSLPVTLGQPASISCRSSQSLVYSDGNTYLNWFRQRPGQSPRRLIYKVSNRDSGVPDRFSGSGSGTDFTLRISRVEAEDVGVYYCMQGTHWPPTFGQGTKLEIKRTV

SEQ ID NO: 273 - protein, 5 aa, Artificial Sequence
  source: /note=“Description of Artificial Sequence: Synthetic peptide”
SYPMN

SEQ ID NO: 274 - protein, 17 aa, Artificial Sequence
  source: /note=“Description of Artificial Sequence: Synthetic peptide”
RISRSGGRTSYADSVKG

SEQ ID NO: 275 - protein, 12 aa, Artificial Sequence
  source: /note=“Description of Artificial Sequence: Synthetic peptide”
DATRRHYNGMDV

SEQ ID NO: 276 - protein, 11 aa, Artificial Sequence
  source: /note=“Description of Artificial Sequence: Synthetic peptide”
RASRSVGKYLA

SEQ ID NO: 277 - protein, 7 aa, Artificial Sequence
  source: /note=“Description of Artificial Sequence: Synthetic peptide”
YASLRAG

SEQ ID NO: 278 - protein, 9 aa, Artificial Sequence
  source: /note=“Description of Artificial Sequence: Synthetic peptide”
QQYGSSPRT

SEQ ID NO: 279 - protein, 30 aa, Artificial Sequence; identical to SEQ ID NO: 87
  source: /note=“Description of Artificial Sequence: Synthetic polypeptide”

SEQ ID NO: 280 - protein, 14 aa, Artificial Sequence
  source: /note=“Description of Artificial Sequence: Synthetic peptide”
WVRQAPGKGLEWVS

SEQ ID NO: 281 - protein, 32 aa, Artificial Sequence; identical to SEQ ID NO: 9
  source: /note=“Description of Artificial Sequence: Synthetic polypeptide”

SEQ ID NO: 282 - protein, 11 aa, Artificial Sequence
  source: /note=“Description of Artificial Sequence: Synthetic peptide”
WGKGTTVTVSS

SEQ ID NO: 283 - protein, 23 aa, Artificial Sequence
  source: /note=“Description of Artificial Sequence: Synthetic peptide”
DIVLTQSPATLSLSPGERASLSC

SEQ ID NO: 284 - protein, 15 aa, Artificial Sequence
  source: /note=“Description of Artificial Sequence: Synthetic peptide”
WYQQKPGQAPRLLFY

SEQ ID NO: 285 - protein, 32 aa, Artificial Sequence; identical to SEQ ID NO: 29
  source: /note=“Description of Artificial Sequence: Synthetic polypeptide”

SEQ ID NO: 286 - protein, 13 aa, Artificial Sequence
  source: /note=“Description of Artificial Sequence: Synthetic peptide”
FGQGTKLEMKRTV

SEQ ID NO: 287 - protein, 121 aa, Artificial Sequence
  source: /note=“Description of Artificial Sequence: Synthetic polypeptide”
EVQLVESGGGLVQPGGSLRLSCAASGFTFSSYPMNWVRQAPGKGLEWVSRISRSGGRTSYADSVKGRFTISRDNSKNTLYLQMNSLRAEDTAVYYCARDATRRHYNGMDVWGKGTTVTVSS

SEQ ID NO: 288 - protein, 110 aa, Artificial Sequence
  source: /note=“Description of Artificial Sequence: Synthetic polypeptide”
DIVLTQSPATLSLSPGERASLSCRASRSVGKYLAWYQQKPGQAPRLLFYYASLRAGDIPSRFTASGSGTDFTLTISRLEPEDFAVYYCQQYGSSPRTFGQGTKLEMKRTV

SEQ ID NO: 289 - protein, 5 aa, Artificial Sequence
  source: /note=“Description of Artificial Sequence: Synthetic peptide”
RYRMH

SEQ ID NO: 290 - protein, 17 aa, Artificial Sequence
  source: /note=“Description of Artificial Sequence: Synthetic peptide”
SIASSGGRTRYADSVKG

SEQ ID NO: 291 - protein, 13 aa, Artificial Sequence
  source: /note=“Description of Artificial Sequence: Synthetic peptide”
GGLPYRGHYGMDV

SEQ ID NO: 292 - protein, 11 aa, Artificial Sequence
  source: /note=“Description of Artificial Sequence: Synthetic peptide”
RASQSISSYLN

SEQ ID NO: 293 - protein, 7 aa, Artificial Sequence
  source: /note=“Description of Artificial Sequence: Synthetic peptide”
VASSLQS

SEQ ID NO: 294 - protein, 9 aa, Artificial Sequence
  source: /note=“Description of Artificial Sequence: Synthetic peptide”
QQARSIPWT

SEQ ID NO: 295 - protein, 30 aa, Artificial Sequence; identical to SEQ ID NO: 87
  source: /note=“Description of Artificial Sequence: Synthetic polypeptide”

SEQ ID NO: 296 - protein, 14 aa, Artificial Sequence
  source: /note=“Description of Artificial Sequence: Synthetic peptide”
WVRQAPGKGLEWVS

SEQ ID NO: 297 - protein, 32 aa, Artificial Sequence; identical to SEQ ID NO: 9
  source: /note=“Description of Artificial Sequence: Synthetic polypeptide”

SEQ ID NO: 298 - protein, 11 aa, Artificial Sequence
  source: /note=“Description of Artificial Sequence: Synthetic peptide”
WGQGTLVTVSS

SEQ ID NO: 299 - protein, 23 aa, Artificial Sequence
  source: /note=“Description of Artificial Sequence: Synthetic peptide”
EIMLTQSPSSLSASVGDRVTITC

SEQ ID NO: 300 - protein, 15 aa, Artificial Sequence
  source: /note=“Description of Artificial Sequence: Synthetic peptide”
WYQQKPGKAPKLLIS

SEQ ID NO: 301 - protein, 32 aa, Artificial Sequence
  source: /note=“Description of Artificial Sequence: Synthetic polypeptide”
GVPSRFSGSRSGTDFTLTISSLQPEDFATYYC

SEQ ID NO: 302 - protein, 13 aa, Artificial Sequence
  source: /note=“Description of Artificial Sequence: Synthetic peptide”
FGQGTNVEIKRTV

SEQ ID NO: 303 - protein, 122 aa, Artificial Sequence
  source: /note=“Description of Artificial Sequence: Synthetic polypeptide”
EVQLVESGGGLVQPGGSLRLSCAASGFTFSRYRMHWVRQAPGKGLEWVSSIASSGGRTRYADSVKGRFTISRDNSKNTLYLQMNSLRAEDTAVYYCARGGLPYRGHYGMDVWGQGTLVTVSS

SEQ ID NO: 304 - protein, 110 aa, Artificial Sequence
  source: /note=“Description of Artificial Sequence: Synthetic polypeptide”
EIMLTQSPSSLSASVGDRVTITCRASQSISSYLNWYQQKPGKAPKLLISVASSLQSGVPSRFSGSRSGTDFTLTISSLQPEDFATYYCQQARSIPWTFGQGTNVEIKRTV

SEQ ID NO: 305 - protein, 5 aa, Artificial Sequence
  source: /note=“Description of Artificial Sequence: Synthetic peptide”
SYAMS

SEQ ID NO: 306 - protein, 17 aa, Artificial Sequence
  source: /note=“Description of Artificial Sequence: Synthetic peptide”
AISGSGGSTYYADSVKG

SEQ ID NO: 307 - protein, 13 aa, Artificial Sequence
  source: /note=“Description of Artificial Sequence: Synthetic peptide”
GGLPYRGHYGMDV

SEQ ID NO: 308 - protein, 16 aa, Artificial Sequence
  source: /note=“Description of Artificial Sequence: Synthetic peptide”
RSSQSLLHSNGYNYVD

SEQ ID NO: 309 - protein, 7 aa, Artificial Sequence
  source: /note=“Description of Artificial Sequence: Synthetic peptide”
LGSNRAS

SEQ ID NO: 310 - protein, 9 aa, Artificial Sequence
  source: /note=“Description of Artificial Sequence: Synthetic peptide”
MQALKIPRT

SEQ ID NO: 311 - protein, 30 aa, Artificial Sequence; identical to SEQ ID NO: 87
  source: /note=“Description of Artificial Sequence: Synthetic polypeptide”

SEQ ID NO: 312 - protein, 14 aa, Artificial Sequence
  source: /note=“Description of Artificial Sequence: Synthetic peptide”
WVRQAPGKGLEWVS

SEQ ID NO: 313 - protein, 32 aa, Artificial Sequence; identical to SEQ ID NO: 9
  source: /note=“Description of Artificial Sequence: Synthetic polypeptide”

SEQ ID NO: 314 - protein, 11 aa, Artificial Sequence
  source: /note=“Description of Artificial Sequence: Synthetic peptide”
WGQGTLVTVSS

SEQ ID NO: 315 - protein, 23 aa, Artificial Sequence
  source: /note=“Description of Artificial Sequence: Synthetic peptide”
DIVLTQSPLSLPVTPGEPASISC

SEQ ID NO: 316 - protein, 15 aa, Artificial Sequence
  source: /note=“Description of Artificial Sequence: Synthetic peptide”
WYLQKPGQSPQLLIY

SEQ ID NO: 317 - protein, 32 aa, Artificial Sequence; identical to SEQ ID NO: 189
  source: /note=“Description of Artificial Sequence: Synthetic polypeptide”

SEQ ID NO: 318 - protein, 13 aa, Artificial Sequence
  source: /note=“Description of Artificial Sequence: Synthetic peptide”
FGQGTKVEIKRTV

SEQ ID NO: 319 - protein, 122 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGGLPYRGHYGMDVWGQGTLVTVSS

SEQ ID NO: 320 - protein, 115 aa, Artificial Sequence
  source: /note=“Description of Artificial Sequence: Synthetic polypeptide”
DIVLTQSPLSLPVTPGEPASISCRSSQSLLHSNGYNYVDWYLQKPGQSPQLLIYLGSNRASGVPDRFSGSGSGTDFTLKISRVEAEDVGVYYCMQALKIPRTFGQGTKVEIKRTV

SEQ ID NO: 321 - protein, 5 aa, Artificial Sequence
  source: /note=“Description of Artificial Sequence: Synthetic peptide”
PYYMV

SEQ ID NO: 322 - protein, 17 aa, Artificial Sequence
  source: /note=“Description of Artificial Sequence: Synthetic peptide”
SINRSGGRTAYADSVKG

SEQ ID NO: 323 - protein, 11 aa, Artificial Sequence
  source: /note=“Description of Artificial Sequence: Synthetic peptide”
AIAAGRYGMDV

SEQ ID NO: 324 - protein, 11 aa, Artificial Sequence
  source: /note=“Description of Artificial Sequence: Synthetic peptide”
RASQSVSSYLA

SEQ ID NO: 325 - protein, 7 aa, Artificial Sequence
  source: /note=“Description of Artificial Sequence: Synthetic peptide”
DASNRAT

SEQ ID NO: 326 - protein, 10 aa, Artificial Sequence
  source: /note=“Description of Artificial Sequence: Synthetic peptide”
QQRTNWPPLT

SEQ ID NO: 327 - protein, 30 aa, Artificial Sequence; identical to SEQ ID NO: 87
  source: /note=“Description of Artificial Sequence: Synthetic polypeptide”

SEQ ID NO: 328 - protein, 14 aa, Artificial Sequence
  source: /note=“Description of Artificial Sequence: Synthetic peptide”
WVRQAPGKGLEWVS

SEQ ID NO: 329 - protein, 32 aa, Artificial Sequence; identical to SEQ ID NO: 9
  source: /note=“Description of Artificial Sequence: Synthetic polypeptide”

SEQ ID NO: 330 - protein, 11 aa, Artificial Sequence
  source: /note=“Description of Artificial Sequence: Synthetic peptide”
WGKGTTVTVSS

SEQ ID NO: 331 - protein, 23 aa, Artificial Sequence
  source: /note=“Description of Artificial Sequence: Synthetic peptide”
DIEMTQSPATLSLSPGERATLSC

SEQ ID NO: 332 - protein, 15 aa, Artificial Sequence
  source: /note=“Description of Artificial Sequence: Synthetic peptide”
WYQQKPGQPPRLLIY

SEQ ID NO: 333 - protein, 32 aa, Artificial Sequence; identical to SEQ ID NO: 205
  source: /note=“Description of Artificial Sequence: Synthetic polypeptide”

SEQ ID NO: 334 - protein, 13 aa, Artificial Sequence
  source: /note=“Description of Artificial Sequence: Synthetic peptide”
FGGGTKVEIKRTV

SEQ ID NO: 335 - protein, 120 aa, Artificial Sequence
  source: /note=“Description of Artificial Sequence: Synthetic polypeptide”
EVQLVESGGGLVQPGGSLRLSCAASGFTFSPYYMVWVRQAPGKGLEWVSSINRSGGRTAYADSVKGRFTISRDNSKNTLYLQMNSLRAEDTAVYYCARAIAAGRYGMDVWGKGTTVTVSS

SEQ ID NO: 336 - protein, 111 aa, Artificial Sequence
  source: /note=“Description of Artificial Sequence: Synthetic polypeptide”
DIEMTQSPATLSLSPGERATLSCRASQSVSSYLAWYQQKPGQPPRLLIYDASNRATGIPARFSGSGSGTDFTLTISSLEPEDFAVYYCQQRTNWPPLTFGGGTKVEIKRTV

SEQ ID NO: 337 - protein, 5 aa, Artificial Sequence
  source: /note=“Description of Artificial Sequence: Synthetic peptide”
RYTMR

SEQ ID NO: 338 - protein, 17 aa, Artificial Sequence
  source: /note=“Description of Artificial Sequence: Synthetic peptide”
GISRSGGRTVYADSVKG

SEQ ID NO: 339 - protein, 14 aa, Artificial Sequence
  source: /note=“Description of Artificial Sequence: Synthetic peptide”
DPFGVVNHFYYMDV

SEQ ID NO: 340 - protein, 11 aa, Artificial Sequence
  source: /note=“Description of Artificial Sequence: Synthetic peptide”
RASQSIHTYLN

SEQ ID NO: 341 - protein, 7 aa, Artificial Sequence
  source: /note=“Description of Artificial Sequence: Synthetic peptide”
GASNLQN

SEQ ID NO: 342 - protein, 9 aa, Artificial Sequence
  source: /note=“Description of Artificial Sequence: Synthetic peptide”
QQTYRTPTT

SEQ ID NO: 343 - protein, 30 aa, Artificial Sequence; identical to SEQ ID NO: 87
  source: /note=“Description of Artificial Sequence: Synthetic polypeptide”

SEQ ID NO: 344 - protein, 14 aa, Artificial Sequence
  source: /note=“Description of Artificial Sequence: Synthetic peptide”
WVRQAPGKGLEWVS

SEQ ID NO: 345 - protein, 32 aa, Artificial Sequence; identical to SEQ ID NO: 9
  source: /note=“Description of Artificial Sequence: Synthetic polypeptide”

SEQ ID NO: 346 - protein, 11 aa, Artificial Sequence
  source: /note=“Description of Artificial Sequence: Synthetic peptide”
WGKGTTVTVSS

SEQ ID NO: 347 - protein, 23 aa, Artificial Sequence
  source: /note=“Description of Artificial Sequence: Synthetic peptide”
EIMLTQSPPSLSASVGDRVTITC

SEQ ID NO: 348 - protein, 15 aa, Artificial Sequence
  source: /note=“Description of Artificial Sequence: Synthetic peptide”
WYQQKPGKAPKLLIY

SEQ ID NO: 349 - protein, 32 aa, Artificial Sequence
  source: /note=“Description of Artificial Sequence: Synthetic polypeptide”
GVPSRFSGTGSGTDFALTISSLQPEDFATYSC

SEQ ID NO: 350 - protein, 13 aa, Artificial Sequence
  source: /note=“Description of Artificial Sequence: Synthetic peptide”
FGPGTKVDIKRTV

SEQ ID NO: 351 - protein, 123 aa, Artificial Sequence
  source: /note=“Description of Artificial Sequence: Synthetic polypeptide”
EVQLVESGGGLVQPGGSLRLSCAASGFTFSRYTMRWVRQAPGKGLEWVSGISRSGGRTVYADSVKGRFTISRDNSKNTLYLQMNSLRAEDTAVYYCARDPFGVVNHFYYMDVWGKGTTVTVSS

SEQ ID NO: 352 - protein, 110 aa, Artificial Sequence
  source: /note=“Description of Artificial Sequence: Synthetic polypeptide”
EIMLTQSPPSLSASVGDRVTITCRASQSIHTYLNWYQQKPGKAPKLLIYGASNLQNGVPSRFSGTGSGTDFALTISSLQPEDFATYSCQQTYRTPTTFGPGTKVDIKRTV

SEQ ID NO: 353 - protein, 5 aa, Artificial Sequence
  source: /note=“Description of Artificial Sequence: Synthetic peptide”
SYRMS

SEQ ID NO: 354 - protein, 17 aa, Artificial Sequence
  source: /note=“Description of Artificial Sequence: Synthetic peptide”
GIGRSGGRTRYADSVKG

SEQ ID NO: 355 - protein, 11 aa, Artificial Sequence
  source: /note=“Description of Artificial Sequence: Synthetic peptide”
AIAAGRYGMDV

SEQ ID NO: 356 - protein, 12 aa, Artificial Sequence
  source: /note=“Description of Artificial Sequence: Synthetic peptide”
RASQSIRNNYLA

SEQ ID NO: 357 - protein, 7 aa, Artificial Sequence
  source: /note=“Description of Artificial Sequence: Synthetic peptide”
GASYRAT

SEQ ID NO: 358 - protein, 9 aa, Artificial Sequence
  source: /note=“Description of Artificial Sequence: Synthetic peptide”
QQRSNWPPT

SEQ ID NO: 359 - protein, 30 aa, Artificial Sequence; identical to SEQ ID NO: 87
  source: /note=“Description of Artificial Sequence: Synthetic polypeptide”

SEQ ID NO: 360 - protein, 14 aa, Artificial Sequence
  source: /note=“Description of Artificial Sequence: Synthetic peptide”
WVRQAPGKGLEWVS

SEQ ID NO: 361 - protein, 32 aa, Artificial Sequence; identical to SEQ ID NO: 9
  source: /note=“Description of Artificial Sequence: Synthetic polypeptide”

SEQ ID NO: 362 - protein, 11 aa, Artificial Sequence
  source: /note=“Description of Artificial Sequence: Synthetic peptide”
WGKGTTVTVSS

SEQ ID NO: 363 - protein, 23 aa, Artificial Sequence
  source: /note=“Description of Artificial Sequence: Synthetic peptide”
DIMLTQSPGTLSLSPGERATLSC

SEQ ID NO: 364 - protein, 15 aa, Artificial Sequence
  source: /note=“Description of Artificial Sequence: Synthetic peptide”
WYQQRPGQAPRLLIY

SEQ ID NO: 365 - protein, 32 aa, Artificial Sequence
  source: /note=“Description of Artificial Sequence: Synthetic polypeptide”
GIPDRFSGSGSGTDFTLTISSLEPEDFAVYYC

SEQ ID NO: 366 - protein, 13 aa, Artificial Sequence
  source: /note=“Description of Artificial Sequence: Synthetic peptide”
FGGGTKVEIKRTV

SEQ ID NO: 367 - protein, 120 aa, Artificial Sequence
  source: /note=“Description of Artificial Sequence: Synthetic polypeptide”
EVQLVESGGGLVQPGGSLRLSCAASGFTFSSYRMSWVRQAPGKGLEWVSGIGRSGGRTRYADSVKGRFTISRDNSKNTLYLQMNSLRAEDTAVYYCARAIAAGRYGMDVWGKGTTVTVSS

SEQ ID NO: 368 - protein, 111 aa, Artificial Sequence
  source: /note=“Description of Artificial Sequence: Synthetic polypeptide”
DIMLTQSPGTLSLSPGERATLSCRASQSIRNNYLAWYQQRPGQAPRLLIYGASYRATGIPDRFSGSGSGTDFTLTISSLEPEDFAVYYCQQRSNWPPTFGGGTKVEIKRTV

SEQ ID NO: 369 - protein, 5 aa, Artificial Sequence
  source: /note=“Description of Artificial Sequence: Synthetic peptide”
RYPMV

SEQ ID NO: 370 - protein, 17 aa, Artificial Sequence
  source: /note=“Description of Artificial Sequence: Synthetic peptide”
RISRSGGRTQYADSVKG

SEQ ID NO: 371 - protein, 12 aa, Artificial Sequence
  source: /note=“Description of Artificial Sequence: Synthetic peptide”
DATGRHYNGMDV

SEQ ID NO: 372 - protein, 11 aa, Artificial Sequence
  source: /note=“Description of Artificial Sequence: Synthetic peptide”
RASQSISSYLN

SEQ ID NO: 373 - protein, 7 aa, Artificial Sequence
  source: /note=“Description of Artificial Sequence: Synthetic peptide”
GASSLQS

SEQ ID NO: 374 - protein, 9 aa, Artificial Sequence
  source: /note=“Description of Artificial Sequence: Synthetic peptide”
QQANSFPLT

SEQ ID NO: 375 - protein, 30 aa, Artificial Sequence; identical to SEQ ID NO: 87
  source: /note=“Description of Artificial Sequence: Synthetic polypeptide”

SEQ ID NO: 376 - protein, 14 aa, Artificial Sequence
  source: /note=“Description of Artificial Sequence: Synthetic peptide”
WVRQAPGKGLEWVS

SEQ ID NO: 377 - protein, 32 aa, Artificial Sequence; identical to SEQ ID NO: 9
  source: /note=“Description of Artificial Sequence: Synthetic polypeptide”

SEQ ID NO: 378 - protein, 11 aa, Artificial Sequence
  source: /note=“Description of Artificial Sequence: Synthetic peptide”
WGQGTLVTVSS

SEQ ID NO: 379 - protein, 23 aa, Artificial Sequence
  source: /note=“Description of Artificial Sequence: Synthetic peptide”
EIAMTQSPSSLSASVGDRVTITC

SEQ ID NO: 380 - protein, 15 aa, Artificial Sequence
  source: /note=“Description of Artificial Sequence: Synthetic peptide”
WYQQKPGKAPKLLIY

SEQ ID NO: 381 - protein, 32 aa, Artificial Sequence; identical to SEQ ID NO: 61
  source: /note=“Description of Artificial Sequence: Synthetic polypeptide”

SEQ ID NO: 382 - protein, 13 aa, Artificial Sequence
  source: /note=“Description of Artificial Sequence: Synthetic peptide”
FGGGTKVEIKRTV

SEQ ID NO: 383 - protein, 121 aa, Artificial Sequence
  source: /note=“Description of Artificial Sequence: Synthetic polypeptide”
EVQLVESGGGLVQPGGSLRLSCAASGFTFSRYPMVWVRQAPGKGLEWVSRISRSGGRTQYADSVKGRFTISRDNSKNTLYLQMNSLRAEDTAVYYCARDATGRHYNGMDVWGQGTLVTVSS

SEQ ID NO: 384 - protein, 110 aa, Artificial Sequence
  source: /note=“Description of Artificial Sequence: Synthetic polypeptide”
EIAMTQSPSSLSASVGDRVTITCRASQSISSYLNWYQQKPGKAPKLLIYGASSLQSGVPSRFSGSGSGTDFTLTISSLQPEDFATYYCQQANSFPLTFGGGTKVEIKRTV

SEQ ID NO: 385 - protein, 5 aa, Artificial Sequence
  source: /note=“Description of Artificial Sequence: Synthetic peptide”
SYRMS

SEQ ID NO: 386 - protein, 17 aa, Artificial Sequence
  source: /note=“Description of Artificial Sequence: Synthetic peptide”
GIGRSGGRTRYADSVKG

SEQ ID NO: 387 - protein, 11 aa, Artificial Sequence
  source: /note=“Description of Artificial Sequence: Synthetic peptide”
AIAAGRYGMDV

SEQ ID NO: 388 - protein, 12 aa, Artificial Sequence
  source: /note=“Description of Artificial Sequence: Synthetic peptide”
RASQSIRNNYLA

SEQ ID NO: 389 - protein, 7 aa, Artificial Sequence
  source: /note=“Description of Artificial Sequence: Synthetic peptide”
GASYRAT

SEQ ID NO: 390 - protein, 9 aa, Artificial Sequence
  source: /note=“Description of Artificial Sequence: Synthetic peptide”
QQRSNWPPT

SEQ ID NO: 391 - protein, 30 aa, Artificial Sequence; identical to SEQ ID NO: 87
  source: /note=“Description of Artificial Sequence: Synthetic polypeptide”

SEQ ID NO: 392 - protein, 14 aa, Artificial Sequence
  source: /note=“Description of Artificial Sequence: Synthetic peptide”
WVRQAPGKGLEWVS

SEQ ID NO: 393 - protein, 32 aa, Artificial Sequence; identical to SEQ ID NO: 9
  source: /note=“Description of Artificial Sequence: Synthetic polypeptide”

SEQ ID NO: 394 - protein, 11 aa, Artificial Sequence
  source: /note=“Description of Artificial Sequence: Synthetic peptide”
WGKGTTVTVSS

SEQ ID NO: 395 - protein, 23 aa, Artificial Sequence
  source: /note=“Description of Artificial Sequence: Synthetic peptide”
EIELTQSPGTLSLSPGERATLSC

SEQ ID NO: 396 - protein, 15 aa, Artificial Sequence
  source: /note=“Description of Artificial Sequence: Synthetic peptide”
WYQQRPGQAPRLLIY

SEQ ID NO: 397 - protein, 32 aa, Artificial Sequence; identical to SEQ ID NO: 365
  source: /note=“Description of Artificial Sequence: Synthetic polypeptide”

SEQ ID NO: 398 - protein, 13 aa, Artificial Sequence
  source: /note=“Description of Artificial Sequence: Synthetic peptide”
FGGGTKVEIKRTV

SEQ ID NO: 399 - protein, 120 aa, Artificial Sequence; identical to SEQ ID NO: 367
  source: /note=“Description of Artificial Sequence: Synthetic polypeptide”

SEQ ID NO: 400 - protein, 111 aa, Artificial Sequence
  source: /note=“Description of Artificial Sequence: Synthetic polypeptide”
EIELTQSPGTLSLSPGERATLSCRASQSIRNNYLAWYQQRPGQAPRLLIYGASYRATGIPDRFSGSGSGTDFTLTISSLEPEDFAVYYCQQRSNWPPTFGGGTKVEIKRTV

SEQ ID NO: 401 - protein, 5 aa, Artificial Sequence
  source: /note=“Description of Artificial Sequence: Synthetic peptide”
RYRMA

SEQ ID NO: 402 - protein, 17 aa, Artificial Sequence
  source: /note=“Description of Artificial Sequence: Synthetic peptide”
GISYSGGETLYADSVKG

SEQ ID NO: 403 - protein, 10 aa, Artificial Sequence
  source: /note=“Description of Artificial Sequence: Synthetic peptide”
DVRWLQGLDN

SEQ ID NO: 404 - protein, 16 aa, Artificial Sequence
  source: /note=“Description of Artificial Sequence: Synthetic peptide”
RSSQSLLHTNGNNYLD

SEQ ID NO: 405 - protein, 7 aa, Artificial Sequence
  source: /note=“Description of Artificial Sequence: Synthetic peptide”
LGSNRAS

SEQ ID NO: 406 - protein, 9 aa, Artificial Sequence
  source: /note=“Description of Artificial Sequence: Synthetic peptide”
MQTLQTPLT

SEQ ID NO: 407 - protein, 30 aa, Artificial Sequence; identical to SEQ ID NO: 87
  source: /note=“Description of Artificial Sequence: Synthetic polypeptide”

SEQ ID NO: 408 - protein, 14 aa, Artificial Sequence
  source: /note=“Description of Artificial Sequence: Synthetic peptide”
WVRQAPGKGLEWVS

SEQ ID NO: 409 - protein, 32 aa, Artificial Sequence; identical to SEQ ID NO: 9
  source: /note=“Description of Artificial Sequence: Synthetic polypeptide”

SEQ ID NO: 410 - protein, 11 aa, Artificial Sequence
  source: /note=“Description of Artificial Sequence: Synthetic peptide”
WGQGTLVTVSS

SEQ ID NO: 411 - protein, 23 aa, Artificial Sequence
  source: /note=“Description of Artificial Sequence: Synthetic peptide”
EIKLTQSPLSLPVTPGEPASISC

SEQ ID NO: 412 - protein, 15 aa, Artificial Sequence
  source: /note=“Description of Artificial Sequence: Synthetic peptide”
WYLQKPGQSPQLLIY

SEQ ID NO: 413 - protein, 32 aa, Artificial Sequence; identical to SEQ ID NO: 189
  source: /note=“Description of Artificial Sequence: Synthetic polypeptide”

SEQ ID NO: 414 - protein, 13 aa, Artificial Sequence
  source: /note=“Description of Artificial Sequence: Synthetic peptide”
FGGGTKVEIKRTV

SEQ ID NO: 415 - protein, 119 aa, Artificial Sequence
  source: /note=“Description of Artificial Sequence: Synthetic polypeptide”
EVQLVESGGGLVQPGGSLRLSCAASGFTFSRYRMAWVRQAPGKGLEWVSGISYSGGETLYADSVKGRFTISRDNSKNTLYLQMNSLRAEDTAVYYCARDVRWLQGLDNWGQGTLVTVSS

SEQ ID NO: 416 - protein, 115 aa, Artificial Sequence
  source: /note=“Description of Artificial Sequence: Synthetic polypeptide”
EIKLTQSPLSLPVTPGEPASISCRSSQSLLHTNGNNYLDWYLQKPGQSPQLLIYLGSNRASGVPDRFSGSGSGTDFTLKISRVEAEDVGVYYCMQTLQTPLTFGGGTKVEIKRTV

SEQ ID NO: 417 - protein, 5 aa, Artificial Sequence
  source: /note=“Description of Artificial Sequence: Synthetic peptide”
SYAMS

SEQ ID NO: 418 - protein, 17 aa, Artificial Sequence
  source: /note=“Description of Artificial Sequence: Synthetic peptide”
AISGSGGSTYYADSVKG

SEQ ID NO: 419 - protein, 9 aa, Artificial Sequence
  source: /note=“Description of Artificial Sequence: Synthetic peptide”
EGRPGYMDV

SEQ ID NO: 420 - protein, 11 aa, Artificial Sequence
  source: /note=“Description of Artificial Sequence: Synthetic peptide”
RTSLSIATYLH

SEQ ID NO: 421 - protein, 7 aa, Artificial Sequence
  source: /note=“Description of Artificial Sequence: Synthetic peptide”
HASSLQT

SEQ ID NO: 422 - protein, 9 aa, Artificial Sequence
  source: /note=“Description of Artificial Sequence: Synthetic peptide”
QQSYSSPYT

SEQ ID NO: 423 - protein, 30 aa, Artificial Sequence; identical to SEQ ID NO: 87
  source: /note=“Description of Artificial Sequence: Synthetic polypeptide”

SEQ ID NO: 424 - protein, 14 aa, Artificial Sequence
  source: /note=“Description of Artificial Sequence: Synthetic peptide”
WVRQAPGKGLEWVS

SEQ ID NO: 425 - protein, 32 aa, Artificial Sequence; identical to SEQ ID NO: 9
  source: /note=“Description of Artificial Sequence: Synthetic polypeptide”

SEQ ID NO: 426 - protein, 11 aa, Artificial Sequence
  source: /note=“Description of Artificial Sequence: Synthetic peptide”
WGQGTLVTVSS

SEQ ID NO: 427 - protein, 23 aa, Artificial Sequence
  source: /note=“Description of Artificial Sequence: Synthetic peptide”
EIVLTQSPSLLSASVGDRVTITC

SEQ ID NO: 428 - protein, 15 aa, Artificial Sequence
  source: /note=“Description of Artificial Sequence: Synthetic peptide”
WYQQKPGRAPKLLIY

SEQ ID NO: 429 - protein, 32 aa, Artificial Sequence
  source: /note=“Description of Artificial Sequence: Synthetic polypeptide”
GVPSRFSGSGSGTDFTLTISSLLPEDFATYFC

SEQ ID NO: 430 - protein, 13 aa, Artificial Sequence
  source: /note=“Description of Artificial Sequence: Synthetic peptide”
FGRGTKLEIKRTV

SEQ ID NO: 431 - protein, 118 aa, Artificial Sequence
  source: /note=“Description of Artificial Sequence: Synthetic polypeptide”
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAREGRPGYMDVWGQGTLVTVSS

SEQ ID NO: 432 - protein, 110 aa, Artificial Sequence
  source: /note=“Description of Artificial Sequence: Synthetic polypeptide”
EIVLTQSPSLLSASVGDRVTITCRTSLSIATYLHWYQQKPGRAPKLLIYHASSLQTGVPSRFSGSGSGTDFTLTISSLLPEDFATYFCQQSYSSPYTFGRGTKLEIKRTV

SEQ ID NO: 433 - protein, 5 aa, Artificial Sequence
  source: /note=“Description of Artificial Sequence: Synthetic peptide”
VYGMI

SEQ ID NO: 434 - protein, 17 aa, Artificial Sequence
  source: /note=“Description of Artificial Sequence: Synthetic peptide”
GIPPSGGVTLYADSVKG

SEQ ID NO: 435 - protein, 7 aa, Artificial Sequence
  source: /note=“Description of Artificial Sequence: Synthetic peptide”
GNYGMDV

SEQ ID NO: 436 - protein, 11 aa, Artificial Sequence
  source: /note=“Description of Artificial Sequence: Synthetic peptide”
RASQSVSSYLA

SEQ ID NO: 437 - protein, 7 aa, Artificial Sequence
  source: /note=“Description of Artificial Sequence: Synthetic peptide”
DASNRAT

SEQ ID NO: 438 - protein, 9 aa, Artificial Sequence
  source: /note=“Description of Artificial Sequence: Synthetic peptide”
QQRSNWPPT

SEQ ID NO: 439 - protein, 30 aa, Artificial Sequence; identical to SEQ ID NO: 87
  source: /note=“Description of Artificial Sequence: Synthetic polypeptide”

SEQ ID NO: 440 - protein, 14 aa, Artificial Sequence
  source: /note=“Description of Artificial Sequence: Synthetic peptide”
WVRQAPGKGLEWVS

SEQ ID NO: 441 - protein, 32 aa, Artificial Sequence; identical to SEQ ID NO: 9
  source: /note=“Description of Artificial Sequence: Synthetic polypeptide”

SEQ ID NO: 442 - protein, 11 aa, Artificial Sequence
  source: /note=“Description of Artificial Sequence: Synthetic peptide”
WGKGTTVTVSS

SEQ ID NO: 443 - protein, 23 aa, Artificial Sequence
  source: /note=“Description of Artificial Sequence: Synthetic peptide”
EIALTQSPATLSLSPGERATLSC

SEQ ID NO: 444 - protein, 15 aa, Artificial Sequence
  source: /note=“Description of Artificial Sequence: Synthetic peptide”
WYQQKPGQAPRLLIY

SEQ ID NO: 445 - protein, 32 aa, Artificial Sequence; identical to SEQ ID NO: 205
  source: /note=“Description of Artificial Sequence: Synthetic polypeptide”

SEQ ID NO: 446 - protein, 13 aa, Artificial Sequence
  source: /note=“Description of Artificial Sequence: Synthetic peptide”
FGGGTKVEIKRTV

SEQ ID NO: 447 - protein, 116 aa, Artificial Sequence
  source: /note=“Description of Artificial Sequence: Synthetic polypeptide”
EVQLVESGGGLVQPGGSLRLSCAASGFTFSVYGMIWVRQAPGKGLEWVSGIPPSGGVTLYADSVKGRFTISRDNSKNTLYLQMNSLRAEDTAVYYCARGNYGMDVWGKGTTVTVSS

SEQ ID NO: 448 - protein, 110 aa, Artificial Sequence
  source: /note=“Description of Artificial Sequence: Synthetic polypeptide”
EIALTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKVEIKRTV

SEQ ID NO: 449-464 - skipped (16 entries)

SEQ ID NO: 465 - protein, 18 aa, Artificial Sequence
  source: /note=“Description of Artificial Sequence: Synthetic peptide”
MHSSALLCCLVLLTGVRA

SEQ ID NO: 466 - protein, 104 aa, Homo sapiens
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 467 - protein, 330 aa, Homo sapiens [sequence omitted]

SEQ ID NO: 468 - protein, 552 aa, Artificial Sequence
  source: /note=“Description of Artificial Sequence: Synthetic polypeptide”
REVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDT

SEQ ID NO: 469 - protein, 8 aa, Artificial Sequence
  source: /note=“Description of Artificial Sequence: Synthetic peptide”
  VARIANT (2)..(2): /replace=“Y”
  VARIANT (3)..(3): /replace=“T”
  VARIANT (4)..(4): /replace=“F” or “I”
  VARIANT (5)..(5): /replace=“S” or “I”
  VARIANT (6)..(6): /replace=“S”
  VARIANT (7)..(7): /replace=“S”
  SITE (1)..(8): /note=“Variant residues given in the sequence have no preference with respect to those in the annotations for variant positions”
GFSLRDYA

SEQ ID NO: 470 - protein, 8 aa, Artificial Sequence
  source: /note=“Description of Artificial Sequence: Synthetic peptide”
  VARIANT (2)..(2): /replace=“T”
  VARIANT (7)..(7): /replace=“S”
  SITE (1)..(8): /note=“Variant residues given in the sequence have no preference with respect to those in the annotations for variant positions”
ISGSGGNT

SEQ ID NO: 471 - protein, 8 aa, Artificial Sequence
  source: /note=“Description of Artificial Sequence: Synthetic peptide”
  VARIANT (1)..(1): /replace=“A” or “S”
  VARIANT (2)..(2): /replace=“L” or “S”
  VARIANT (3)..(3): /replace=“A” or “Y” or “V” or “G” or “S”
  VARIANT (4)..(4): /replace=“L” or “S”
  VARIANT (5)..(5): /replace=“Y” or “P” or “T” or “I” or “N” or “K” or “H” or “Q”
  VARIANT (6)..(6): /replace=“V” or “F” or “I” or “G” or “R” or “H”
  VARIANT (7)..(7): /replace=“Y” or “V” or “Any acidic residue (D/E)”
  VARIANT (8)..(8): /replace=“A” or “T” or “P” or “K” or “S” or “Q”
  SITE (1)..(8): /note=“Variant residues given in the sequence have no preference with respect to those in the annotations for variant positions”
ERWRLLAY

SEQ ID NO: 472 - protein, 8 aa, Artificial Sequence
  source: /note=“Description of Artificial Sequence: Synthetic peptide”
GFTFSTYG

SEQ ID NO: 473 - protein, 8 aa, Artificial Sequence
  source: /note=“Description of Artificial Sequence: Synthetic peptide”
IWDDGSYK

SEQ ID NO: 474 - protein, 11 aa, Artificial Sequence
  source: /note=“Description of Artificial Sequence: Synthetic peptide”
  VARIANT (2)..(2): /replace=“G”
  VARIANT (6)..(6): /replace=“Y”
  VARIANT (8)..(8): /replace=“D” or “S”
  VARIANT (9)..(9): /replace=“L” or “M”
  VARIANT (11)..(11): /replace=“I” or “S”
  SITE (1)..(11): /note=“Variant residues given in the sequence have no preference with respect to those in the annotations for variant positions”
AVGYSDSYRAL

SEQ ID NO: 475 - protein, 8 aa, Artificial Sequence
  source: /note=“Description of Artificial Sequence: Synthetic peptide”
GYTFTNDI

SEQ ID NO: 476 - protein, 8 aa, Artificial Sequence
  source: /note=“Description of Artificial Sequence: Synthetic peptide”
INAGYGNT

SEQ ID NO: 477 - protein, 13 aa, Artificial Sequence
  source: /note=“Description of Artificial Sequence: Synthetic peptide”
  VARIANT (11)..(11): /replace=“I”
  VARIANT (13)..(13): /replace=“Y”
  SITE (1)..(13): /note=“Variant residues given in the sequence have no preference with respect to those in the annotations for variant positions”
ARGYYRSPTWFDW

SEQ ID NO: 478 - protein, 8 aa, Artificial Sequence
  source: /note=“Description of Artificial Sequence: Synthetic peptide”
GFTFSTYG

SEQ ID NO: 479 - protein, 8 aa, Artificial Sequence
  source: /note=“Description of Artificial Sequence: Synthetic peptide”
IWDDGSYK

SEQ ID NO: 480 - protein, 13 aa, Artificial Sequence
  source: /note=“Description of Artificial Sequence: Synthetic peptide”
  VARIANT (4)..(4): /replace=“C”
  VARIANT (6)..(6): /replace=“G”
  VARIANT (8)..(8): /replace=“G”
  VARIANT (13)..(13): /replace=“I”
  SITE (1)..(13): /note=“Variant residues given in the sequence have no preference with respect to those in the annotations for variant positions”
ARDGSVWVYAFDL

SEQ ID NO: 481 - protein, 8 aa, Artificial Sequence
  source: /note=“Description of Artificial Sequence: Synthetic peptide”
  VARIANT (4)..(4): /replace=“F”
  VARIANT (5)..(5): /replace=“S”
  VARIANT (7)..(7): /replace=“Y”
  SITE (1)..(8): /note=“Variant residues given in the sequence have no preference with respect to those in the annotations for variant positions”
GFTVNSFA

SEQ ID NO: 482 - protein, 8 aa, Artificial Sequence
  source: /note=“Description of Artificial Sequence: Synthetic peptide”
  VARIANT (2)..(2): /replace=“T”
  VARIANT (7)..(7): /replace=“G”
  SITE (1)..(8): /note=“Variant residues given in the sequence have no preference with respect to those in the annotations for variant positions”
ISGSGGST

SEQ ID NO: 483 - protein, 12 aa, Artificial Sequence
  source: /note=“Description of Artificial Sequence: Synthetic peptide”
  MOD_RES (3)..(3): Any acidic residue (D/E)
  MOD_RES (5)..(5): Any acidic residue (D/E)
  VARIANT (11)..(11): /replace=“Any acidic residue (D/E)”
  VARIANT (12)..(12): /replace=“I”
  SITE (1)..(12): /note=“Variant residues given in the sequence have no preference with respect to those in the annotations for variant positions”
ARXVXGYGAFAL

SEQ ID NO: 484 - protein, 8 aa, Artificial Sequence
  source: /note=“Description of Artificial Sequence: Synthetic peptide”
  VARIANT (5)..(5): /replace=“N” or “S”
  VARIANT (6)..(6): /replace=“S”
  VARIANT (7)..(7): /replace=“Y”
  SITE (1)..(8): /note=“Variant residues given in the sequence have no preference with respect to those in the annotations for variant positions”
GFTFRDFA

SEQ ID NO: 485 - protein, 8 aa, Artificial Sequence
  source: /note=“Description of Artificial Sequence: Synthetic peptide”
  VARIANT (2)..(2): /replace=“T”
  VARIANT (7)..(7): /replace=“S” or “G”
  SITE (1)..(8): /note=“Variant residues given in the sequence have no preference with respect to those in the annotations for variant positions”
ISGSGGNT

SEQ ID NO: 486 - protein, 15 aa, Artificial Sequence
  source: /note=“Description of Artificial Sequence: Synthetic peptide”
  VARIANT (2)..(2): /replace=“S”
  VARIANT (3)..(3): /replace=“Any acidic residue (D/E)”
  VARIANT (7)..(7): /replace=“G”
  VARIANT (12)..(12): /replace=“Y”
  VARIANT (13)..(13): /replace=“F” or “I”
  VARIANT (15)..(15): /replace=“Y” or “H” or “S”
  SITE (1)..(15): /note=“Variant residues given in the sequence have no preference with respect to those in the annotations for variant positions”
ARVGWEVRTPGDLDD

SEQ ID NO: 487 - protein, 8 aa, Artificial Sequence
  source: /note=“Description of Artificial Sequence: Synthetic peptide”
  VARIANT (7)..(7): /replace=“S”
  SITE (1)..(8): /note=“Variant residues given in the sequence have no preference with respect to those in the annotations for variant positions”
GFTFSSYA

SEQ ID NO: 488 - protein, 8 aa, Artificial Sequence
  source: /note=“Description of Artificial Sequence: Synthetic peptide”
ITGSGGST

SEQ ID NO: 489 - protein, 9 aa, Artificial Sequence
  source: /note=“Description of Artificial Sequence: Synthetic peptide”
  VARIANT (3)..(3): /replace=“G”
  VARIANT (4)..(4): /replace=“ ”
  VARIANT (5)..(5): /replace=“C”
  VARIANT (6)..(6): /replace=“G” or “S”
  MOD_RES (8)..(8): Any acidic residue(D/E)
  VARIANT (9)..(9): /replace=“Y” or “I” or “V”
  SITE (1)..(9): /note=“Variant residues given in the sequence have no preference with respect to those in the annotations for variant positions”
ARDRGALXL

SEQ ID NO: 490 - protein, 8 aa, Artificial Sequence
  source: /note=“Description of Artificial Sequence: Synthetic peptide”
GFTFSSYS

SEQ ID NO: 491 - protein, 8 aa, Artificial Sequence
  source: /note=“Description of Artificial Sequence: Synthetic peptide”
ISSSSSYI

SEQ ID NO: 492 - protein, 15 aa, Artificial Sequence
  source: /note=“Description of Artificial Sequence: Synthetic peptide”
  VARIANT (1)..(1): /replace=“G”
  VARIANT (3)..(3): /replace=“Q”
  VARIANT (4)..(4): /replace=“D”
  VARIANT (5)..(5): /replace=“A” or “Any acidic residue (D/E)”
  VARIANT (6)..(6): /replace=“L”
  VARIANT (7)..(7): /replace=“L”
  VARIANT (8)..(8): /replace=“R” or “I”
  VARIANT (9)..(9): /replace=“V” or “G”
  VARIANT (10)..(10): /replace=“C” or “L”
  VARIANT (12)..(12): /replace=“C” or “G”
  VARIANT (13)..(13): /replace=“F” or “V”
  VARIANT (14)..(14): /replace=“A”
  VARIANT (15)..(15): /replace=“P” or “T”
  SITE (1)..(15): /note=“Variant residues given in the sequence have no preference with respect to those in the annotations for variant positions”
CRPYYFWSCWDWYDH

SEQ ID NO: 493 - protein, 8 aa, Artificial Sequence
  source: /note=“Description of Artificial Sequence: Synthetic peptide”
  VARIANT (4)..(4): /replace=“C”
  VARIANT (5)..(5): /replace=“N” or “S”
  VARIANT (6)..(6): /replace=“S”
  VARIANT (7)..(7): /replace=“Y”
  SITE (1)..(8): /note=“Variant residues given in the sequence have no preference with respect to those in the annotations for variant positions”
GFTFRDFA

SEQ ID NO: 494 - protein, 8 aa, Artificial Sequence
  source: /note=“Description of Artificial Sequence: Synthetic peptide”
  VARIANT (2)..(2): /replace=“T”
  VARIANT (7)..(7): /replace=“S” or “G”
  SITE (1)..(8): /note=“Variant residues given in the sequence have no preference with respect to those in the annotations for variant positions”
ISGSGGNT

SEQ ID NO: 495 - protein, 15 aa, Artificial Sequence
  source: /note=“Description of Artificial Sequence: Synthetic peptide”
  VARIANT (1)..(1): /replace=“A” or “S”
  VARIANT (2)..(2): /replace=“S” or “I”
  VARIANT (3)..(3): /replace=“G”
  VARIANT (4)..(4): /replace=“Any acidic residue (D/E)”
  VARIANT (5)..(5): /replace=“Y” or “S”
  VARIANT (6)..(6): /replace=“S”
  VARIANT (7)..(7): /replace=“S” or “I”
  VARIANT (8)..(8): /replace=“G” or “C”
  VARIANT (9)..(9): /replace=“W” or “G” or “C”
  VARIANT (10)..(10): /replace=“H” or “T”
  VARIANT (11)..(11): /replace=“S” or “I”
  VARIANT (12)..(12): /replace=“W” or “C” or “G” or “R”
  VARIANT (13)..(13): /replace=“F” or “V” or “C”
  VARIANT (14)..(14): /replace=“D” or “A”
  VARIANT (15)..(15): /replace=“H” or “S” or “T”
  SITE (1)..(15): /note=“Variant residues given in the sequence have no preference with respect to those in the annotations for variant positions”
ERVADYRVLPRLLYP

SEQ ID NO: 496 - protein, 8 aa, Artificial Sequence
  source: /note=“Description of Artificial Sequence: Synthetic peptide”
GFTFSSYA

SEQ ID NO: 497 - protein, 8 aa, Artificial Sequence
  source: /note=“Description of Artificial Sequence: Synthetic peptide”
ITGSGGST

SEQ ID NO: 498 - protein, 8 aa, Artificial Sequence
  source: /note=“Description of Artificial Sequence: Synthetic peptide”
  VARIANT (1)..(1): /replace=“W”
  VARIANT (2)..(2): /replace=“P” or “R” or “Y”
  VARIANT (3)..(3): /replace=“H” or “P” or “S”
  VARIANT (4)..(4): /replace=“S”
  VARIANT (5)..(5): /replace=“I” or “N”
  VARIANT (6)..(6): /replace=“P”
  VARIANT (8)..(8): /replace=“W”
  SITE (1)..(8): /note=“Variant residues given in the sequence have no preference with respect to those in the annotations for variant positions”
AFDRDLDS

SEQ ID NO: 499 - protein, 8 aa, Artificial Sequence
  source: /note=“Description of Artificial Sequence: Synthetic peptide”
  VARIANT (7)..(7): /replace=“N” or “D”
  SITE (1)..(8): /note=“Variant residues given in the sequence have no preference with respect to those in the annotations for variant positions”
GGTFSSYG

SEQ ID NO: 500 - protein, 8 aa, Artificial Sequence
  source: /note=“Description of Artificial Sequence: Synthetic peptide”
IIPIFGTA

SEQ ID NO: 501 - protein, 9 aa, Artificial Sequence
  source: /note=“Description of Artificial Sequence: Synthetic peptide”
  VARIANT (1)..(1): /replace=“E” or “A”
  VARIANT (2)..(2): /replace=“R” or “I”
  VARIANT (3)..(3): /replace=“A” or “Any acidic residue (D/E)”
  VARIANT (4)..(4): /replace=“S” or “F” or “D”
  VARIANT (5)..(5): /replace=“C” or “Any aromatic residue (F/Y/W)”
  VARIANT (6)..(6): /replace=“A” or “Any acidic residue (D/E)”
  VARIANT (8)..(8): /replace=“V” or “G”
  VARIANT (9)..(9): /replace=“D”
  SITE (1)..(9): /note=“Variant residues given in the sequence have no preference with respect to those in the annotations for variant positions”
SSYYSYSCY

SEQ ID NO: 502 - protein, 8 aa, Artificial Sequence
  source: /note=“Description of Artificial Sequence: Synthetic peptide”
  VARIANT (1)..(1): /replace=“V” or “G”
  VARIANT (2)..(2): /replace=“S”
  VARIANT (3)..(3): /replace=“T”
  VARIANT (4)..(4): /replace=“F” or “L” or “I”
  VARIANT (5)..(5): /replace=“T”
  VARIANT (6)..(6): /replace=“N” or “K”
  VARIANT (8)..(8): /replace=“S”
  SITE (1)..(8): /note=“Variant residues given in the sequence have no preference with respect to those in the annotations for variant positions”
CYPYNHYY

SEQ ID NO: 503 - protein, 8 aa, Artificial Sequence
  source: /note=“Description of Artificial Sequence: Synthetic peptide”
INPYTGSA

SEQ ID NO: 504 - protein, 14 aa, Artificial Sequence
  source: /note=“Description of Artificial Sequence: Synthetic peptide”
  VARIANT (1)..(1): /replace=“E” or “A”
  VARIANT (2)..(2): /replace=“R” or “K” or “G”
  VARIANT (3)..(3): /replace=“N” or “A” or “Any acidic residue (D/E)”
  VARIANT (4)..(4): /replace=“D” or “T” or “A”
  VARIANT (5)..(5): /replace=“K” or “T”
  VARIANT (6)..(6): /replace=“R” or “V” or “G”
  VARIANT (7)..(7): /replace=“H” or “R” or “L”
  VARIANT (8)..(8): /replace=“N” or “P” or “L” or “Q”
  VARIANT (9)..(9): /replace=“S” or “D”
  VARIANT (10)..(10): /replace=“N” or “K” or “I” or “T”
  VARIANT (11)..(11): /replace=“G” or “V” or “C” or “A”
  VARIANT (12)..(12): /replace=“R” or “L” or “I”
  VARIANT (13)..(13): /replace=“N” or “G” or “V” or “Y” or “A” or “Any acidic residue (D/E)”
  VARIANT (14)..(14): /replace=“V” or “F” or “L” or “A”
  SITE (1)..(14): /note=“Variant residues given in the sequence have no preference with respect to those in the annotations for variant positions”
SSHSPESHYHSMHI

SEQ ID NO: 505 - protein, 8 aa, Artificial Sequence
  source: /note=“Description of Artificial Sequence: Synthetic peptide”
  VARIANT (4)..(4): /replace=“F” or “V”
  SITE (1)..(8): /note=“Variant residues given in the sequence have no preference with respect to those in the annotations for variant positions”
GFTCNSFA

SEQ ID NO: 506 - protein, 8 aa, Artificial Sequence
  source: /note=“Description of Artificial Sequence: Synthetic peptide”
ISGSGGGT

SEQ ID NO: 507 - protein, 16 aa, Artificial Sequence
  source: /note=“Description of Artificial Sequence: Synthetic peptide”
  VARIANT (1)..(1): /replace=“A”
  VARIANT (2)..(2): /replace=“K” or “T” or “Q”
  VARIANT (4)..(4): /replace=“R” or “L”
  VARIANT (7)..(7): /replace=“S” or “D”
  VARIANT (8)..(8): /replace=“Any acidic residue (D/E)”
  VARIANT (9)..(9): /replace=“L”
  VARIANT (10)..(10): /replace=“L” or “G”
  VARIANT (14)..(14): /replace=“L” or “I”
  VARIANT (16)..(16): /replace=“S” or “D”
  SITE (1)..(16): /note=“Variant residues given in the sequence have no preference with respect to those in the annotations for variant positions”
ENDSSPYYFWSGAFDY

SEQ ID NO: 508 - protein, 8 aa, Artificial Sequence
  source: /note=“Description of Artificial Sequence: Synthetic peptide”
  VARIANT (1)..(1): /replace=“G”
  VARIANT (5)..(5): /replace=“S” or “R”
  VARIANT (6)..(6): /replace=“N” or “D”
  VARIANT (7)..(7): /replace=“S” or “F”
  VARIANT (8)..(8): /replace=“A”
  SITE (1)..(8): /note=“Variant residues given in the sequence have no preference with respect to those in the annotations for variant positions”
CFTFNSYS

SEQ ID NO: 509 - protein, 8 aa, Artificial Sequence
  source: /note=“Description of Artificial Sequence: Synthetic peptide”
  VARIANT (2)..(2): /replace=“T”
  VARIANT (7)..(7): /replace=“N” or “T” or “G”
  SITE (1)..(8): /note=“Variant residues given in the sequence have no preference with respect to those in the annotations for variant positions”
ISGSGGST

SEQ ID NO: 510 - protein, 16 aa, Artificial Sequence
  source: /note=“Description of Artificial Sequence: Synthetic peptide”
  VARIANT (1)..(1): /replace=“A”
  VARIANT (2)..(2): /replace=“G”
  VARIANT (4)..(4): /replace=“Q”
  VARIANT (5)..(5): /replace=“D”
  VARIANT (6)..(6): /replace=“Any acidic residue (D/E)”
  VARIANT (8)..(8): /replace=“L” or “G”
  VARIANT (9)..(9): /replace=“R” or “I”
  VARIANT (10)..(10): /replace=“V” or “G”
  VARIANT (11)..(11): /replace=“C” or “G”
  VARIANT (13)..(13): /replace=“C” or “G”
  VARIANT (14)..(14): /replace=“L” or “V”
  VARIANT (15)..(15): /replace=“Any acidic residue (D/E)”
  VARIANT (16)..(16): /replace=“P” or “T”
  SITE (1)..(16): /note=“Variant residues given in the sequence have no preference with respect to those in the annotations for variant positions”
ECRPYYFWSCWDWFAH

SEQ ID NO: 511 - protein, 8 aa, Artificial Sequence
  source: /note=“Description of Artificial Sequence: Synthetic peptide”
  VARIANT (1)..(1): /replace=“G”
  VARIANT (2)..(2): /replace=“F” or “L”
  VARIANT (5)..(5): /replace=“S” or “R”
  VARIANT (6)..(6): /replace=“N” or “D”
  VARIANT (7)..(7): /replace=“S” or “F”
  VARIANT (8)..(8): /replace=“D” or “A”
  SITE (1)..(8): /note=“Variant residues given in the sequence have no preference with respect to those in the annotations for variant positions”
VYTFNSYS

SEQ ID NO: 512 - protein, 8 aa, Artificial Sequence
  source: /note=“Description of Artificial Sequence: Synthetic peptide”
  VARIANT (2)..(2): /replace=“T”
  VARIANT (5)..(5): /replace=“G”
  VARIANT (7)..(7): /replace=“N” or “T” or “G”
  SITE (1)..(8): /note=“Variant residues given in the sequence have no preference with respect to those in the annotations for variant positions”
ISGSSGST

SEQ ID NO: 513 - protein, 14 aa, Artificial Sequence
  source: /note=“Description of Artificial Sequence: Synthetic peptide”
  VARIANT (1)..(1): /replace=“E” or “A”
  VARIANT (2)..(2): /replace=“K” or “T”
  VARIANT (3)..(3): /replace=“Any acidic residue (D/E)”
  VARIANT (4)..(4): /replace=“T” or “A”
  VARIANT (5)..(5): /replace=“T”
  VARIANT (6)..(6): /replace=“R” or “G”
  VARIANT (7)..(7): /replace=“R” or “L”
  VARIANT (8)..(8): /replace=“P”
  VARIANT (9)..(9): /replace=“D”
  VARIANT (10)..(10): /replace=“N” or “K” or “I” or “Y” or “T”
  VARIANT (13)..(13): /replace=“V” or “Any acidic residue (D/E)”
  VARIANT (14)..(14): /replace=“I” or “V”
  SITE (1)..(14): /note=“Variant residues given in the sequence have no preference with respect to those in the annotations for variant positions”
SRNDKEHHYSGMYG

SEQ ID NO: 514 - protein, 8 aa, Artificial Sequence
  source: /note=“Description of Artificial Sequence: Synthetic peptide”
  VARIANT (5)..(5): /replace=“T”
  MOD_RES (6)..(6): Any acidic residue (D/E)
  SITE (1)..(8): /note=“Variant residues given in the sequence have no preference with respect to those in the annotations for variant positions”
GYTLSXLS

SEQ ID NO: 515 - protein, 8 aa, Artificial Sequence
  source: /note=“Description of Artificial Sequence: Synthetic peptide”
  VARIANT (4)..(4): /replace=“Q”
  MOD_RES (5)..(5): Any acidic residue (D/E)
  VARIANT (6)..(6): /replace=“G”
  VARIANT (8)..(8): /replace=“T”
  SITE (1)..(8): /note=“Variant residues given in the sequence have no preference with respect to those in the annotations for variant positions”
FDPEXNEI

SEQ ID NO: 516 - protein, 22 aa, Artificial Sequence
  source: /note=“Description of Artificial Sequence: Synthetic peptide”
  VARIANT (2)..(2): /replace=“K” or “T”
  VARIANT (3)..(3): /replace=“D”
  VARIANT (4)..(4): /replace=“V”
  VARIANT (5)..(5): /replace=“V” or “G”
  VARIANT (7)..(7): /replace=“S” or “D”
  VARIANT (8)..(8): /replace=“S” or “D”
  VARIANT (9)..(9): /replace=“Any acidic residue (D/E)”
  VARIANT (10)..(10): /replace=“R” or “I”
  VARIANT (11)..(11): /replace=“R”
  VARIANT (12)..(12): /replace=“G”
  VARIANT (14)..(14): /replace=“S” or “D”
  VARIANT (16)..(16): /replace=“L”
  VARIANT (17)..(17): /replace=“T”
  VARIANT (18)..(18): /replace=“S” or “D”
  VARIANT (19)..(19): /replace=“G”
  VARIANT (20)..(20): /replace=“I”
  SITE (1)..(22): /note=“Variant residues given in the sequence have no preference with respect to those in the annotations for variant positions”
ANYLWYYYYSSVYYGRNYVMDV

SEQ ID NO: 517 - protein, 8 aa, Artificial Sequence
  source: /note=“Description of Artificial Sequence: Synthetic peptide”
  VARIANT (4)..(4): /replace=“L”
  VARIANT (6)..(6): /replace=“R” or “N”
  VARIANT (8)..(8): /replace=“G”
  SITE (1)..(8): /note=“Variant residues given in the sequence have no preference with respect to those in the annotations for variant positions”
GYTFTSYS

SEQ ID NO: 518 - protein, 8 aa, Artificial Sequence
  source: /note=“Description of Artificial Sequence: Synthetic peptide”
  VARIANT (1)..(1): /replace=“V”
  VARIANT (3)..(3): /replace=“F” or “V” or “A”
  VARIANT (4)..(4): /replace=“N”
  VARIANT (5)..(5): /replace=“N”
  VARIANT (7)..(7): /replace=“D”
  SITE (1)..(8): /note=“Variant residues given in the sequence have no preference with respect to those in the annotations for variant positions”
ISTYSGNT

SEQ ID NO: 519 - protein, 22 aa, Artificial Sequence
  source: /note=“Description of Artificial Sequence: Synthetic peptide”
  VARIANT (1)..(1): /replace=“A”
  VARIANT (3)..(3): /replace=“D”
  VARIANT (4)..(4): /replace=“L” or “V”
  VARIANT (5)..(5): /replace=“G”
  VARIANT (6)..(6): /replace=“D”
  VARIANT (7)..(7): /replace=“D”
  VARIANT (8)..(8): /replace=“S” or “D”
  MOD_RES (9)..(9): Any acidic residue (D/E)
  VARIANT (10)..(10): /replace=“R”
  VARIANT (11)..(11): /replace=“R” or “N”
  VARIANT (12)..(12): /replace=“G”
  VARIANT (13)..(13): /replace=“D”
  VARIANT (14)..(14): /replace=“S” or “D”
  VARIANT (15)..(15): /replace=“G”
  VARIANT (16)..(16): /replace=“L”
  VARIANT (17)..(17): /replace=“T”
  VARIANT (18)..(18): /replace=“D”
  VARIANT (19)..(19): /replace=“C” or “G”
  VARIANT (20)..(20): /replace=“L” or “I”
  VARIANT (22)..(22): /replace=“I” or “V”
  SITE (1)..(22): /note=“Variant residues given in the sequence have no preference with respect to those in the annotations for variant positions”
EKYFVYYYXSSVYYRRNYSMDF

SEQ ID NO: 520 - protein, 8 aa, Artificial Sequence
  source: /note=“Description of Artificial Sequence: Synthetic peptide”
  VARIANT (1)..(1): /replace=“G”
  VARIANT (2)..(2): /replace=“L”
  VARIANT (4)..(4): /replace=“F” or “V”
  VARIANT (5)..(5): /replace=“D”
  VARIANT (8)..(8): /replace=“A”
  SITE (1)..(8): /note=“Variant residues given in the sequence have no preference with respect to those in the annotations for variant positions”
RFTCNDYS

SEQ ID NO: 521 - protein, 8 aa, Artificial Sequence
  source: /note=“Description of Artificial Sequence: Synthetic peptide”
  VARIANT (2)..(2): /replace=“T”
  VARIANT (7)..(7): /replace=“H” or “R”
  SITE (1)..(8): /note=“Variant residues given in the sequence have no preference with respect to those in the annotations for variant positions”
ISWNSGSI

SEQ ID NO: 522 - protein, 16 aa, Artificial Sequence
  source: /note=“Description of Artificial Sequence: Synthetic peptide”
  VARIANT (1)..(1): /replace=“A”
  VARIANT (2)..(2): /replace=“G”
  VARIANT (3)..(3): /replace=“L”
  VARIANT (4)..(4): /replace=“Q”
  VARIANT (5)..(5): /replace=“D”
  MOD_RES (6)..(6): Any acidic residue (D/E)
  VARIANT (8)..(8): /replace=“G”
  VARIANT (9)..(9): /replace=“R”
  VARIANT (10)..(10): /replace=“G”
  VARIANT (11)..(11): /replace=“L” or “C” or “Any aromatic residue (F/Y/W)”
  VARIANT (12)..(12): /replace=“Any acidic residue (D/E)”
  VARIANT (13)..(13): /replace=“G”
  VARIANT (14)..(14): /replace=“F” or “V”
  VARIANT (16)..(16): /replace=“P”
  SITE (1)..(16): /note=“Variant residues given in the sequence have no preference with respect to those in the annotations for variant positions”
ECRPYXFWSCGHWCDL

SEQ ID NO: 523 - protein, 8 aa, Artificial Sequence
  source: /note=“Description of Artificial Sequence: Synthetic peptide”
  VARIANT (8)..(8): /replace=“A”
  SITE (1)..(8): /note=“Variant residues given in the sequence have no preference with respect to those in the annotations for variant positions”
RFTFDDYS

SEQ ID NO: 524 - protein, 8 aa, Artificial Sequence
  source: /note=“Description of Artificial Sequence: Synthetic peptide”
ISWNSGRI

SEQ ID NO: 525 - protein, 11 aa, Artificial Sequence
  source: /note=“Description of Artificial Sequence: Synthetic peptide”
  VARIANT (5)..(5): /replace=“D”
  VARIANT (7)..(7): /replace=“F” or “V”
  VARIANT (8)..(8): /replace=“S” or “D”
  VARIANT (9)..(9): /replace=“F” or “L”
  VARIANT (11)..(11): /replace=“S” or “D”
  SITE (1)..(11): /note=“Variant residues given in the sequence have no preference with respect to those in the annotations for variant positions”
ARYGYPCYCDY

SEQ ID NO: 526 - protein, 8 aa, Artificial Sequence
  source: /note=“Description of Artificial Sequence: Synthetic peptide”
  VARIANT (1)..(1): /replace=“G”
  VARIANT (2)..(2): /replace=“S”
  VARIANT (3)..(3): /replace=“S”
  VARIANT (5)..(5): /replace=“R”
  VARIANT (8)..(8): /replace=“C” or “L”
  SITE (1)..(8): /note=“Variant residues given in the sequence have no preference with respect to those in the annotations for variant positions”
VYYFSNYW

SEQ ID NO: 527 - protein, 8 aa, Artificial Sequence
  source: /note=“Description of Artificial Sequence: Synthetic peptide”
IDPSNSYT

SEQ ID NO: 528 - protein, 14 aa, Artificial Sequence
  source: /note=“Description of Artificial Sequence: Synthetic peptide”
  VARIANT (2)..(2): /replace=“R”
  VARIANT (3)..(3): /replace=“Any acidic residue (D/E)”
  VARIANT (4)..(4): /replace=“L” or “I” or “A”
  VARIANT (5)..(5): /replace=“T” or “A”
  VARIANT (6)..(6): /replace=“T” or “G”
  VARIANT (7)..(7): /replace=“C” or “R” or “G”
  VARIANT (8)..(8): /replace=“H” or “N”
  VARIANT (9)..(9): /replace=“S” or “D”
  VARIANT (10)..(10): /replace=“K” or “ ” or “Y” or “T”
  VARIANT (11)..(11): /replace=“G”
  VARIANT (12)..(12): /replace=“R”
  VARIANT (14)..(14): /replace=“V” or “G”
  SITE (1)..(14): /note=“Variant residues given in the sequence have no preference with respect to those in the annotations for variant positions”
ASARKASRYNCMDF

SEQ ID NO: 529 - protein, 8 aa, Artificial Sequence
  source: /note=“Description of Artificial Sequence: Synthetic peptide”
  VARIANT (5)..(5): /replace=“N”
  VARIANT (6)..(6): /replace=“N”
  VARIANT (7)..(7): /replace=“F”
  VARIANT (8)..(8): /replace=“A”
  SITE (1)..(8): /note=“Variant residues given in the sequence have no preference with respect to those in the annotations for variant positions”
GFTFSSYS

SEQ ID NO: 530 - protein, 8 aa, Artificial Sequence
  source: /note=“Description of Artificial Sequence: Synthetic peptide”
  VARIANT (2)..(2): /replace=“N” or “T”
  VARIANT (3)..(3): /replace=“G”
  VARIANT (5)..(5): /replace=“G”
  VARIANT (6)..(6): /replace=“G”
  VARIANT (7)..(7): /replace=“R” or “T” or “G”
  SITE (1)..(8): /note=“Variant residues given in the sequence have no preference with respect to those in the annotations for variant positions”
ISASSTST

SEQ ID NO: 531 - protein, 22 aa, Artificial Sequence
  source: /note=“Description of Artificial Sequence: Synthetic peptide”
  VARIANT (2)..(2): /replace=“T”
  VARIANT (7)..(7): /replace=“D”
  VARIANT (8)..(8): /replace=“D”
  MOD_RES (9)..(9): Any acidic residue (D/E)
  VARIANT (10)..(10): /replace=“R” or “I”
  VARIANT (11)..(11): /replace=“R”
  VARIANT (14)..(14): /replace=“S”
  VARIANT (15)..(15): /replace=“R” or “G”
  VARIANT (16)..(16): /replace=“R”
  VARIANT (17)..(17): /replace=“K” or “T”
  VARIANT (18)..(18): /replace=“S” or “D”
  VARIANT (20)..(20): /replace=“I”
  VARIANT (21)..(21): /replace=“D”
  SITE (1)..(22): /note=“Variant residues given in the sequence have no preference with respect to those in the annotations for variant positions”
AKDLGYYYXSSGYYEHNYGMNV

SEQ ID NO: 532 - protein, 8 aa, Artificial Sequence
  source: /note=“Description of Artificial Sequence: Synthetic peptide”
  VARIANT (5)..(5): /replace=“S” or “R”
  VARIANT (6)..(6): /replace=“D”
  VARIANT (7)..(7): /replace=“F”
  VARIANT (8)..(8): /replace=“A”
  SITE (1)..(8): /note=“Variant residues given in the sequence have no preference with respect to those in the annotations for variant positions”
GFTFNSYS

SEQ ID NO: 533 - protein, 8 aa, Artificial Sequence
  source: /note=“Description of Artificial Sequence: Synthetic peptide”
  VARIANT (2)..(2): /replace=“N” or “T”
  VARIANT (3)..(3): /replace=“G”
  VARIANT (5)..(5): /replace=“G”
  VARIANT (6)..(6): /replace=“G”
  VARIANT (7)..(7): /replace=“N” or “G” or “R” or “T”
  SITE (1)..(8): /note=“Variant residues given in the sequence have no preference with respect to those in the annotations for variant positions”
ISASSTST

SEQ ID NO: 534 - protein, 22 aa, Artificial Sequence
  source: /note=“Description of Artificial Sequence: Synthetic peptide”
  VARIANT (2)..(2): /replace=“R”
  MOD_RES (5)..(5): Any acidic residue (D/E)
  VARIANT (7)..(7): /replace=“S” or “D”
  VARIANT (8)..(8): /replace=“T” or “A”
  VARIANT (10)..(10): /replace=“V” or “G”
  VARIANT (11)..(11): /replace=“R”
  VARIANT (12)..(12): /replace=“S”
  VARIANT (15)..(15): /replace=“P”
  VARIANT (16)..(16): /replace=“R”
  VARIANT (18)..(18): /replace=“D”
  VARIANT (19)..(19): /replace=“D” or “G”
  VARIANT (20)..(20): /replace=“L”
  VARIANT (21)..(21): /replace=“D”
  VARIANT (22)..(22): /replace=“V”
  SITE (1)..(22): /note=“Variant residues given in the sequence have no preference with respect to those in the annotations for variant positions”
ASRGXYYSSESYYRHHRYCMNI

SEQ ID NO: 535 - protein, 8 aa, Artificial Sequence
  source: /note=“Description of Artificial Sequence: Synthetic peptide”
  VARIANT (2)..(2): /replace=“S”
  VARIANT (3)..(3): /replace=“T”
  SITE (1)..(8): /note=“Variant residues given in the sequence have no preference with respect to those in the annotations for variant positions”
GYNFTRYG

SEQ ID NO: 536 - protein, 8 aa, Artificial Sequence
  source: /note=“Description of Artificial Sequence: Synthetic peptide”
ISTYSGNT

SEQ ID NO: 537 - protein, 14 aa, Artificial Sequence
  source: /note=“Description of Artificial Sequence: Synthetic peptide”
  VARIANT (1)..(1): /replace=“A”
  VARIANT (3)..(3): /replace=“S” or “Any acidic residue (D/E)”
  VARIANT (4)..(4): /replace=“L” or “I” or “A”
  VARIANT (5)..(5): /replace=“T” or “A”
  VARIANT (6)..(6): /replace=“A” or “T” or “G”
  VARIANT (7)..(7): /replace=“R” or “V” or “D” or “C”
  VARIANT (8)..(8): /replace=“H” or “R” or “Q”
  VARIANT (9)..(9): /replace=“S” or “D”
  VARIANT (10)..(10): /replace=“N” or “ ”
  VARIANT (11)..(11): /replace=“V” or “G”
  VARIANT (12)..(12): /replace=“I”
  MOD_RES (13)..(13): Any acidic residue (D/E)
  VARIANT (14)..(14): /replace=“V”
  SITE (1)..(14): /note=“Variant residues given in the sequence have no preference with respect to those in the annotations for variant positions”
SRARSSGYYYCMXI

SEQ ID NO: 538 - protein, 8 aa, Artificial Sequence
  source: /note=“Description of Artificial Sequence: Synthetic peptide”
  VARIANT (2)..(2): /replace=“V”
  SITE (1)..(8): /note=“Variant residues given in the sequence have no preference with respect to those in the annotations for variant positions”
GFTFSTYG

SEQ ID NO: 539 - protein, 8 aa, Artificial Sequence
  source: /note=“Description of Artificial Sequence: Synthetic peptide”
IWDDGSYK

SEQ ID NO: 540 - protein, 15 aa, Artificial Sequence
  source: /note=“Description of Artificial Sequence: Synthetic peptide”
  VARIANT (2)..(2): /replace=“R” or “I”
  VARIANT (3)..(3): /replace=“A”
  VARIANT (4)..(4): /replace=“R”
  VARIANT (5)..(5): /replace=“S”
  VARIANT (6)..(6): /replace=“T”
  VARIANT (7)..(7): /replace=“R” or “L” or “I”
  VARIANT (8)..(8): /replace=“D” or “A”
  VARIANT (9)..(9): /replace=“L”
  VARIANT (10)..(10): /replace=“L” or “I”
  VARIANT (11)..(11): /replace=“V” or “G”
  VARIANT (12)..(12): /replace=“C” or “L” or “G”
  VARIANT (13)..(13): /replace=“L” or “V”
  VARIANT (15)..(15): /replace=“P” or “T”
  SITE (1)..(15): /note=“Variant residues given in the sequence have no preference with respect to those in the annotations for variant positions”
ASSMYPMSRRWWFDH

SEQ ID NO: 541 - protein, 8 aa, Artificial Sequence
  source: /note=“Description of Artificial Sequence: Synthetic peptide”
  VARIANT (5)..(5): /replace=“S” or “R”
  VARIANT (6)..(6): /replace=“D”
  VARIANT (7)..(7): /replace=“F”
  SITE (1)..(8): /note=“Variant residues given in the sequence have no preference with respect to those in the annotations for variant positions”
GFTFNSYA

SEQ ID NO: 542 - protein, 8 aa, Artificial Sequence
  source: /note=“Description of Artificial Sequence: Synthetic peptide”
  VARIANT (2)..(2): /replace=“N” or “T”
  VARIANT (3)..(3): /replace=“G”
  VARIANT (6)..(6): /replace=“G”
  VARIANT (7)..(7): /replace=“R” or “N” or “G”
  SITE (1)..(8): /note=“Variant residues given in the sequence have no preference with respect to those in the annotations for variant positions”
ISASGTST

SEQ ID NO: 543 - protein, 22 aa, Artificial Sequence
  source: /note=“Description of Artificial Sequence: Synthetic peptide”
  VARIANT (5)..(5): /replace=“N” or “T”
  VARIANT (9)..(9): /replace=“K” or “Q”
  VARIANT (13)..(13): /replace=“G”
  VARIANT (15)..(15): /replace=“D”
  VARIANT (17)..(17): /replace=“S” or “D”
  VARIANT (18)..(18): /replace=“P”
  VARIANT (21)..(21): /replace=“D”
  SITE (1)..(22): /note=“Variant residues given in the sequence have no preference with respect to those in the annotations for variant positions”
ARDSHVASTGRGVVYHYHGMNV

SEQ ID NO: 544 - protein, 8 aa, Artificial Sequence
  source: /note=“Description of Artificial Sequence: Synthetic peptide”
  VARIANT (1)..(1): /replace=“G”
  VARIANT (5)..(5): /replace=“D”
  VARIANT (6)..(6): /replace=“D”
  VARIANT (8)..(8): /replace=“A”
  SITE (1)..(8): /note=“Variant residues given in the sequence have no preference with respect to those in the annotations for variant positions”
RFTFNYYS

SEQ ID NO: 545 - protein, 8 aa, Artificial Sequence
  source: /note=“Description of Artificial Sequence: Synthetic peptide”
  VARIANT (7)..(7): /replace=“R”
  SITE (1)..(8): /note=“Variant residues given in the sequence have no preference with respect to those in the annotations for variant positions”
ISWNSGSI

SEQ ID NO: 546 - protein, 8 aa, Artificial Sequence
  source: /note=“Description of Artificial Sequence: Synthetic peptide”
  VARIANT (2)..(2): /replace=“L”
  VARIANT (3)..(3): /replace=“V” or “G”
  VARIANT (5)..(5): /replace=“H” or “N” or “Q”
  VARIANT (6)..(6): /replace=“L” or “V”
  VARIANT (8)..(8): /replace=“K” or “Y” or “Q” or “T” or “A”
  SITE (1)..(8): /note=“Variant residues given in the sequence have no preference with respect to those in the annotations for variant positions”
ARSRTRDS

SEQ ID NO: 547 - protein, 8 aa, Artificial Sequence
  source: /note=“Description of Artificial Sequence: Synthetic peptide”
GYTFTNYY

SEQ ID NO: 548 - protein, 8 aa, Artificial Sequence
  source: /note=“Description of Artificial Sequence: Synthetic peptide”
INPYTGSA

SEQ ID NO: 549 - protein, 16 aa, Artificial Sequence
  source: /note=“Description of Artificial Sequence: Synthetic peptide”
  VARIANT (5)..(5): /replace=“F” or “L” or “V”
  VARIANT (7)..(7): /replace=“G”
  VARIANT (11)..(11): /replace=“L”
  VARIANT (13)..(13): /replace=“S” or “D”
  VARIANT (14)..(14): /replace=“R” or “L” or “I”
  VARIANT (16)..(16): /replace=“G”
  SITE (1)..(16): /note=“Variant residues given in the sequence have no preference with respect to those in the annotations for variant positions”
ARDPCGVVNHFYYMDV

SEQ ID NO: 550 - protein, 8 aa, Artificial Sequence
  source: /note=“Description of Artificial Sequence: Synthetic peptide”
GGSFSGYY

SEQ ID NO: 551 - protein, 7 aa, Artificial Sequence
  source: /note=“Description of Artificial Sequence: Synthetic peptide”
INHSGST

SEQ ID NO: 552 - protein, 17 aa, Artificial Sequence
  source: /note=“Description of Artificial Sequence: Synthetic peptide”
  VARIANT (1)..(1): /replace=“A”
  VARIANT (2)..(2): /replace=“R”
  VARIANT (4)..(4): /replace=“S” or “D”
  VARIANT (5)..(5): /replace=“N” or “T”
  VARIANT (6)..(6): /replace=“R”
  VARIANT (7)..(7): /replace=“A”
  VARIANT (8)..(8): /replace=“C” or “L”
  VARIANT (11)..(11): /replace=“S” or “D”
  VARIANT (12)..(12): /replace=“S” or “D”
  VARIANT (13)..(13): /replace=“S” or “D”
  VARIANT (14)..(14): /replace=“G”
  VARIANT (15)..(15): /replace=“R” or “L”
  VARIANT (17)..(17): /replace=“V”
  SITE (1)..(17): /note=“Variant residues given in the sequence have no preference with respect to those in the annotations for variant positions”
EMPYHSSWESYYYVMDF

SEQ ID NO: 553 - protein, 8 aa, Artificial Sequence
  source: /note=“Description of Artificial Sequence: Synthetic peptide”
GYTFTNYY

SEQ ID NO: 554 - protein, 8 aa, Artificial Sequence
  source: /note=“Description of Artificial Sequence: Synthetic peptide”
INPYTGSA

SEQ ID NO: 555 - protein, 13 aa, Artificial Sequence
  source: /note=“Description of Artificial Sequence: Synthetic peptide”
  VARIANT (3)..(3): /replace=“F” or “V” or “A”
  VARIANT (4)..(4): /replace=“L” or “I”
  VARIANT (5)..(5): /replace=“A”
  VARIANT (6)..(6): /replace=“T” or “A”
  VARIANT (7)..(7): /replace=“I” or “G”
  VARIANT (8)..(8): /replace=“L”
  VARIANT (9)..(9): /replace=“S” or “D”
  VARIANT (10)..(10): /replace=“D” or “V” or “G”
  VARIANT (11)..(11): /replace=“R” or “I”
  VARIANT (12)..(12): /replace=“D”
  VARIANT (13)..(13): /replace=“V”
  SITE (1)..(13): /note=“Variant residues given in the sequence have no preference with respect to those in the annotations for variant positions”
ARSRGSCRYCMNI

SEQ ID NO: 556 - protein, 8 aa, Artificial Sequence
  source: /note=“Description of Artificial Sequence: Synthetic peptide”
  VARIANT (4)..(4): /replace=“V”
  SITE (1)..(8): /note=“Variant residues given in the sequence have no preference with respect to those in the annotations for variant positions”
GFTFNSFA

SEQ ID NO: 557 - protein, 8 aa, Artificial Sequence
  source: /note=“Description of Artificial Sequence: Synthetic peptide”
ISGSGGGT

SEQ ID NO: 558 - protein, 8 aa, Artificial Sequence
  source: /note=“Description of Artificial Sequence: Synthetic peptide”
  VARIANT (2)..(2): /replace=“L”
  VARIANT (3)..(3): /replace=“W” or “G”
  VARIANT (4)..(4): /replace=“L”
  VARIANT (5)..(5): /replace=“H” or “N” or “Q”
  VARIANT (6)..(6): /replace=“R” or “I” or “V” or “L”
  MOD_RES (7)..(7): Any acidic residue (D/E)
  VARIANT (8)..(8): /replace=“K” or “T”
  SITE (1)..(8): /note=“Variant residues given in the sequence have no preference with respect to those in the annotations for variant positions”
ARSRTGXS

SEQ ID NO: 559 - protein, 8 aa, Artificial Sequence
  source: /note=“Description of Artificial Sequence: Synthetic peptide”
  VARIANT (2)..(2): /replace=“S”
  VARIANT (8)..(8): /replace=“G”
  SITE (1)..(8): /note=“Variant residues given in the sequence have no preference with respect to those in the annotations for variant positions”
GYTFTRYC

SEQ ID NO: 560 - protein, 8 aa, Artificial Sequence
  source: /note=“Description of Artificial Sequence: Synthetic peptide”
ISTYSGNT

SEQ ID NO: 561 - protein, 15 aa, Artificial Sequence
  source: /note=“Description of Artificial Sequence: Synthetic peptide”
  VARIANT (2)..(2): /replace=“K”
  VARIANT (4)..(4): /replace=“V” or “G”
  VARIANT (5)..(5): /replace=“L”
  VARIANT (7)..(7): /replace=“S” or “D”
  VARIANT (9)..(9): /replace=“V” or “G”
  VARIANT (11)..(11): /replace=“D”
  VARIANT (12)..(12): /replace=“D” or “G”
  VARIANT (13)..(13): /replace=“I”
  VARIANT (14)..(14): /replace=“Any acidic residue (D/E)”
  VARIANT (15)..(15): /replace=“I” or “V”
  SITE (1)..(15): /note=“Variant residues given in the sequence have no preference with respect to those in the annotations for variant positions”
ARGWRPYRWHYCMNF

SEQ ID NO: 562 - protein, 6 aa, Artificial Sequence
  source: /note=“Description of Artificial Sequence: Synthetic peptide”
  VARIANT (1)..(1): /replace=“Q” or “P” or “H”
  VARIANT (2)..(2): /replace=“V” or “I” or “R”
  VARIANT (3)..(3): /replace=“S” or “L” or “I” or “M”
  VARIANT (5)..(5): /replace=“N” or “T”
  SITE (1)..(6): /note=“Variant residues given in the sequence have no preference with respect to those in the annotations for variant positions”
KSNHKY

SEQ ID NO: 563 - protein, 3 aa, Artificial Sequence
  source: /note=“Description of Artificial Sequence: Synthetic peptide”
AAS

SEQ ID NO: 564 - protein, 9 aa, Artificial Sequence
  source: /note=“Description of Artificial Sequence: Synthetic peptide”
  VARIANT (1)..(1): /replace=“Q” or “K” or “H”
  VARIANT (2)..(2): /replace=“Q” or “K” or “H”
  VARIANT (3)..(3): /replace=“I” or “R”
  VARIANT (4)..(4): /replace=“F”
  VARIANT (5)..(5): /replace=“N” or “S” or “T”
  VARIANT (6)..(6): /replace=“N” or “S” or “T”
  VARIANT (8)..(8): /replace=“I” or “R”
  VARIANT (9)..(9): /replace=“N” or “S” or “T”
  SITE (1)..(9): /note=“Variant residues given in the sequence have no preference with respect to those in the annotations for variant positions”
LLSYPPPLP

SEQ ID NO: 565 - protein, 6 aa, Artificial Sequence
  source: /note=“Description of Artificial Sequence: Synthetic peptide”
QGISTF

SEQ ID NO: 566 - protein, 3 aa, Artificial Sequence
  source: /note=“Description of Artificial Sequence: Synthetic peptide”
AAS

SEQ ID NO: 567 - protein, 9 aa, Artificial Sequence
  source: /note=“Description of Artificial Sequence: Synthetic peptide”
QQTYSTQWT

SEQ ID NO: 568 - protein, 6 aa, Artificial Sequence
  source: /note=“Description of Artificial Sequence: Synthetic peptide”
QSISSW

SEQ ID NO: 569 - protein, 3 aa, Artificial Sequence
  source: /note=“Description of Artificial Sequence: Synthetic peptide”
KAS

SEQ ID NO: 570 - protein, 9 aa, Artificial Sequence
  source: /note=“Description of Artificial Sequence: Synthetic peptide”
QQYGDYPYT

SEQ ID NO: 571 - protein, 6 aa, Artificial Sequence
  source: /note=“Description of Artificial Sequence: Synthetic peptide”
  VARIANT (1)..(1): /replace=“Q” or “P” or “H”
  VARIANT (3)..(3): /replace=“N” or “I” or “L”
  VARIANT (4)..(4): /replace=“I” or “R”
  MOD_RES (5)..(5): Any acidic residue (D/E)
  SITE (1)..(6): /note=“Variant residues given in the sequence have no preference with respect to those in the annotations for variant positions”
KDSSXY

SEQ ID NO: 572 - protein, 3 aa, Artificial Sequence
  source: /note=“Description of Artificial Sequence: Synthetic peptide”
DAA

SEQ ID NO: 573 - protein, 9 aa, Artificial Sequence
  source: /note=“Description of Artificial Sequence: Synthetic peptide”
  VARIANT (1)..(1): /replace=“Q” or “K” or “H”
  VARIANT (2)..(2): /replace=“K” or “H” or “L”
  VARIANT (3)..(3): /replace=“S” or “D” or “F”
  VARIANT (4)..(4): /replace=“A” or “Any acidic residue (D/E)”
  VARIANT (5)..(5): /replace=“I” or “R”
  VARIANT (6)..(6): /replace=“F” or “V”
  VARIANT (7)..(7): /replace=“P” or “T”
  VARIANT (8)..(8): /replace=“S” or “F” or “D”
  VARIANT (9)..(9): /replace=“N” or “S” or “T”
  SITE (1)..(9): /note=“Variant residues given in the sequence have no preference with respect to those in the annotations for variant positions”
LQYVSLHYP

SEQ ID NO: 574 - protein, 6 aa, Artificial Sequence
  source: /note=“Description of Artificial Sequence: Synthetic peptide”
  VARIANT (1)..(1): /replace=“P” or “Any basic residue (R/H/K)”
  VARIANT (2)..(2): /replace=“N” or “T”
  VARIANT (3)..(3): /replace=“S” or “L” or “I” or “M”
  VARIANT (4)..(4): /replace=“R” or “I” or “S”
  VARIANT (5)..(5): /replace=“N” or “D” or “S” or “K” or “T” or “I”
  SITE (1)..(6): /note=“Variant residues given in the sequence have no preference with respect to those in the annotations for variant positions”
QSNHHY

SEQ ID NO: 575 - protein, 3 aa, Artificial Sequence
  source: /note=“Description of Artificial Sequence: Synthetic peptide”
GAS

SEQ ID NO: 576 - protein, 9 aa, Artificial Sequence
  source: /note=“Description of Artificial Sequence: Synthetic peptide”
  VARIANT (1)..(1): /replace=“Q” or “K” or “L” or “P”
  VARIANT (2)..(2): /replace=“E” or “Q” or “K” or “L” or “P”
  VARIANT (3)..(3): /replace=“A” or “S” or “T” or “P”
  VARIANT (4)..(4): /replace=“S” or “V” or “L” or “F”
  VARIANT (5)..(5): /replace=“I” or “R”
  VARIANT (6)..(6): /replace=“A” or “Any acidic residue (D/E)”
  VARIANT (7)..(7): /replace=“Q” or “K” or “R” or “T” or “P”
  VARIANT (8)..(8): /replace=“F”
  VARIANT (9)..(9): /replace=“N” or “S” or “T”
  SITE (1)..(9): /note=“Variant residues given in the sequence have no preference with respect to those in the annotations for variant positions”
HHNYSVAYP

SEQ ID NO: 577 - protein, 6 aa, Artificial Sequence
  source: /note=“Description of Artificial Sequence: Synthetic peptide”
HRIFSY

SEQ ID NO: 578 - protein, 3 aa, Artificial Sequence
  source: /note=“Description of Artificial Sequence: Synthetic peptide”
GAS

SEQ ID NO: 579 - protein, 9 aa, Artificial Sequence
  source: /note=“Description of Artificial Sequence: Synthetic peptide”
QQSFSDPYT

SEQ ID NO: 580 - protein, 6 aa, Artificial Sequence
  source: /note=“Description of Artificial Sequence: Synthetic peptide”
  VARIANT (1)..(1): /replace=“Q” or “P” or “H”
  VARIANT (2)..(2): /replace=“V” or “I” or “R”
  VARIANT (3)..(3): /replace=“S” or “L” or “I” or “M”
  VARIANT (5)..(5): /replace=“N” or “T”
  SITE (1)..(6): /note=“Variant residues given in the sequence have no preference with respect to those in the annotations for variant positions”
KSNHKY

SEQ ID NO: 581 - protein, 3 aa, Artificial Sequence
  source: /note=“Description of Artificial Sequence: Synthetic peptide”
SAS

SEQ ID NO: 582 - protein, 9 aa, Artificial Sequence
  source: /note=“Description of Artificial Sequence: Synthetic peptide”
  VARIANT (1)..(1): /replace=“Q” or “K” or “H”
  VARIANT (2)..(2): /replace=“Q” or “K” or “H”
  VARIANT (3)..(3): /replace=“I” or “R”
  VARIANT (4)..(4): /replace=“F”
  VARIANT (5)..(5): /replace=“N” or “S” or “T”
  VARIANT (6)..(6): /replace=“N” or “S” or “T”
  VARIANT (8)..(8): /replace=“I” or “R”
  VARIANT (9)..(9): /replace=“N” or “S” or “T”
  SITE (1)..(9): /note=“Variant residues given in the sequence have no preference with respect to those in the annotations for variant positions”
LLSYPPPLP

SEQ ID NO: 583 - protein, 6 aa, Artificial Sequence
  source: /note=“Description of Artificial Sequence: Synthetic peptide”
  VARIANT (1)..(1): /replace=“L” or “P” or “Any basic residue (R/H/K)”
  VARIANT (2)..(2): /replace=“S” or “G” or “R” or “T” or “I”
  VARIANT (3)..(3): /replace=“S” or “L” or “V” or “T” or “I”
  VARIANT (4)..(4): /replace=“N” or “S” or “G” or “R” or “T” or “I”
  VARIANT (5)..(5): /replace=“A” or “S” or “E” or “T” or “P” or “I”
  VARIANT (6)..(6): /replace=“D” or “S” or “Y”
  SITE (1)..(6): /note=“Variant residues given in the sequence have no preference with respect to those in the annotations for variant positions”
QDNHNQ

SEQ ID NO: 584 - protein, 3 aa, Artificial Sequence
  source: /note=“Description of Artificial Sequence: Synthetic peptide”
DAS

SEQ ID NO: 585 - protein, 10 aa, Artificial Sequence
  source: /note=“Description of Artificial Sequence: Synthetic peptide”
  VARIANT (1)..(1): /replace=“E” or “Q” or “L” or “P” or “Any basic residue (R/H/K)”
  VARIANT (2)..(2): /replace=“E” or “Q” or “L” or “P” or “Any basic residue (R/H/K)”
  VARIANT (3)..(3): /replace=“G” or “R” or “T” or “I”
  VARIANT (4)..(4): /replace=“H” or “D” or “S” or “F”
  VARIANT (5)..(5): /replace=“G” or “R” or “T” or “I” or “M”
  VARIANT (6)..(6): /replace=“A” or “S” or “T” or “P” or “I”
  VARIANT (7)..(7): /replace=“L” or “V” or “R” or “I”
  VARIANT (8)..(8): /replace=“S” or “Q” or “T” or “P” or “Any basic residue (R/H/K)”
  VARIANT (9)..(9): /replace=“H” or “N” or “S” or “F” or “Any acidic residue (D/E)”
  VARIANT (10)..(10): /replace=“A” or “S” or “T” or “P”
  SITE (1)..(10): /note=“Variant residues given in the sequence have no preference with respect to those in the annotations for variant positions”
NNSYSNHAYN

SEQ ID NO: 586 - protein, 6 aa, Artificial Sequence
  source: /note=“Description of Artificial Sequence: Synthetic peptide”
  VARIANT (1)..(1): /replace=“R” or “L”
  VARIANT (2)..(2): /replace=“S” or “N” or “T”
  VARIANT (3)..(3): /replace=“F” or “L”
  VARIANT (4)..(4): /replace=“D”
  VARIANT (5)..(5): /replace=“S” or “K” or “T” or “P”
  VARIANT (6)..(6): /replace=“L” or “V” or “F” or “I”
  SITE (1)..(6): /note=“Variant residues given in the sequence have no preference with respect to those in the annotations for variant positions”
QAVGAY

SEQ ID NO: 587 - protein, 3 aa, Artificial Sequence
  source: /note=“Description of Artificial Sequence: Synthetic peptide”
GVS

SEQ ID NO: 588 - protein, 9 aa, Artificial Sequence
  source: /note=“Description of Artificial Sequence: Synthetic peptide”
  VARIANT (1)..(1): /replace=“Q” or “L” or “H”
  VARIANT (2)..(2): /replace=“K” or “H” or “L”
  VARIANT (3)..(3): /replace=“I” or “T” or “R”
  VARIANT (4)..(4): /replace=“H” or “D” or “Q”
  VARIANT (5)..(5): /replace=“A” or “Any acidic residue (D/E)”
  VARIANT (6)..(6): /replace=“G” or “V” or “L” or “F”
  VARIANT (8)..(8): /replace=“R” or “L”
  VARIANT (9)..(9): /replace=“S” or “P” or “T”
  SITE (1)..(9): /note=“Variant residues given in the sequence have no preference with respect to those in the annotations for variant positions”
KQSNVAPGK

SEQ ID NO: 589 - protein, 6 aa, Artificial Sequence
  source: /note=“Description of Artificial Sequence: Synthetic peptide”
  VARIANT (1)..(1): /replace=“Q” or “P” or “H”
  VARIANT (2)..(2): /replace=“V” or “I” or “R”
  VARIANT (3)..(3): /replace=“S” or “L” or “I” or “M”
  VARIANT (5)..(5): /replace=“N” or “T”
  SITE (1)..(6): /note=“Variant residues given in the sequence have no preference with respect to those in the annotations for variant positions”
KSNHKY

SEQ ID NO: 590 - protein, 3 aa, Artificial Sequence
  source: /note=“Description of Artificial Sequence: Synthetic peptide”
AAS

SEQ ID NO: 591 - protein, 9 aa, Artificial Sequence
  source: /note=“Description of Artificial Sequence: Synthetic peptide”
  VARIANT (1)..(1): /replace=“Q” or “K” or “H”
  VARIANT (2)..(2): /replace=“Q” or “K” or “H”
  VARIANT (3)..(3): /replace=“I” or “R”
  VARIANT (4)..(4): /replace=“F”
  VARIANT (5)..(5): /replace=“N” or “S” or “T”
  VARIANT (6)..(6): /replace=“N” or “S” or “T”
  VARIANT (8)..(8): /replace=“I” or “R”
  VARIANT (9)..(9): /replace=“N” or “S” or “T”
  SITE (1)..(9): /note=“Variant residues given in the sequence have no preference with respect to those in the annotations for variant positions”
LLSYPPPLP

SEQ ID NO: 592-600 - skipped (9 entries)

SEQ ID NO: 601 - protein, 8 aa, Artificial Sequence
  source: /note=“Description of Artificial Sequence: Synthetic peptide”
GFTFSTYG

SEQ ID NO: 602 - protein, 8 aa, Artificial Sequence
  source: /note=“Description of Artificial Sequence: Synthetic peptide”
IWDDGSYK

SEQ ID NO: 603 - protein, 8 aa, Artificial Sequence
  source: /note=“Description of Artificial Sequence: Synthetic peptide”
IWDGGKYK

SEQ ID NO: 604 - protein, 8 aa, Artificial Sequence
  source: /note=“Description of Artificial Sequence: Synthetic peptide”
IWDGGSYK

SEQ ID NO: 605 - protein, 13 aa, Artificial Sequence
  source: /note=“Description of Artificial Sequence: Synthetic peptide”
ARDGSGWGYAFDI

SEQ ID NO: 606 - protein, 25 aa, Artificial Sequence
  source: /note=“Description of Artificial Sequence: Synthetic peptide”
QVQLVESGGGVVQPGRSLRLSCAAS

SEQ ID NO: 607 - protein, 17 aa, Artificial Sequence
  source: /note=“Description of Artificial Sequence: Synthetic peptide”
MHWVRQAPGKGLEWVAV

SEQ ID NO: 608 - protein, 38 aa, Artificial Sequence
  source: /note=“Description of Artificial Sequence: Synthetic polypeptide”
YYGDSVKGRFTISRDNSKNTLYLQMNSLRAEDTAVYYC

SEQ ID NO: 609 - protein, 11 aa, Artificial Sequence
  source: /note=“Description of Artificial Sequence: Synthetic peptide”
WGQGTMVTVSS

SEQ ID NO: 610 - protein, 11 aa, Artificial Sequence
  source: /note=“Description of Artificial Sequence: Synthetic peptide”
WGQGTLVTVSS

SEQ ID NO: 611 - protein, 6 aa, Artificial Sequence
  source: /note=“Description of Artificial Sequence: Synthetic peptide”
QDISEY

SEQ ID NO: 612 - protein, 6 aa, Artificial Sequence
  source: /note=“Description of Artificial Sequence: Synthetic peptide”
QDISKP

SEQ ID NO: 613 - protein, 6 aa, Artificial Sequence
  source: /note=“Description of Artificial Sequence: Synthetic peptide”
QDITKG

SEQ ID NO: 614 - protein, 6 aa, Artificial Sequence
  source: /note=“Description of Artificial Sequence: Synthetic peptide”
QDIGPW

SEQ ID NO: 615 - protein, 6 aa, Artificial Sequence
  source: /note=“Description of Artificial Sequence: Synthetic peptide”
QDIEWW

SEQ ID NO: 616 - protein, 6 aa, Artificial Sequence
  source: /note=“Description of Artificial Sequence: Synthetic peptide”
QDIGAY

SEQ ID NO: 617 - protein, 6 aa, Artificial Sequence
  source: /note=“Description of Artificial Sequence: Synthetic peptide”
QDIEWA

SEQ ID NO: 618 - protein, 6 aa, Artificial Sequence
  source: /note=“Description of Artificial Sequence: Synthetic peptide”
QDIMKT

SEQ ID NO: 619 - protein, 6 aa, Artificial Sequence
  source: /note=“Description of Artificial Sequence: Synthetic peptide”
QDISPY

SEQ ID NO: 620 - protein, 6 aa, Artificial Sequence
  source: /note=“Description of Artificial Sequence: Synthetic peptide”
QDISPW

SEQ ID NO: 621 - protein, 6 aa, Artificial Sequence
  source: /note=“Description of Artificial Sequence: Synthetic peptide”
QDIGPY

SEQ ID NO: 622 - protein, 6 aa, Artificial Sequence
  source: /note=“Description of Artificial Sequence: Synthetic peptide”
QDISYN

SEQ ID NO: 623 - protein, 3 aa, Artificial Sequence
  source: /note=“Description of Artificial Sequence: Synthetic peptide”
DAA

SEQ ID NO: 624 - protein, 3 aa, Artificial Sequence
  source: /note=“Description of Artificial Sequence: Synthetic peptide”
RSP

SEQ ID NO: 625 - protein, 3 aa, Artificial Sequence
  source: /note=“Description of Artificial Sequence: Synthetic peptide”
GSP

SEQ ID NO: 626 - protein, 3 aa, Artificial Sequence
  source: /note=“Description of Artificial Sequence: Synthetic peptide”
ARD

SEQ ID NO: 627 - protein, 3 aa, Artificial Sequence
  source: /note=“Description of Artificial Sequence: Synthetic peptide”
TSA

SEQ ID NO: 628 - protein, 3 aa, Artificial Sequence
  source: /note=“Description of Artificial Sequence: Synthetic peptide”
GAD

SEQ ID NO: 629 - protein, 3 aa, Artificial Sequence
  source: /note=“Description of Artificial Sequence: Synthetic peptide”
DAP

SEQ ID NO: 630 - protein, 3 aa, Artificial Sequence
  source: /note=“Description of Artificial Sequence: Synthetic peptide”
SQP

SEQ ID NO: 631 - protein, 9 aa, Artificial Sequence
  source: /note=“Description of Artificial Sequence: Synthetic peptide”
QQYDSFPYT

SEQ ID NO: 632 - protein, 26 aa, Artificial Sequence
  source: /note=“Description of Artificial Sequence: Synthetic peptide”
DIQLTQSPSSLSASVGDRVTITCQAS

SEQ ID NO: 633 - protein, 26 aa, Artificial Sequence
  source: /note=“Description of Artificial Sequence: Synthetic peptide”
DIQMTQSPSSLSASVGDRVTITCQAS

SEQ ID NO: 634 - protein, 17 aa, Artificial Sequence
  source: /note=“Description of Artificial Sequence: Synthetic peptide”
LNWYQHKPGKAPNPLVF

SEQ ID NO: 635 - protein, 17 aa, Artificial Sequence
  source: /note=“Description of Artificial Sequence: Synthetic peptide”
LNWYQQKPGKAPKLLIY

SEQ ID NO: 636 - protein, 17 aa, Artificial Sequence
  source: /note=“Description of Artificial Sequence: Synthetic peptide”
LNWYQHKPGKAPKPLIY

SEQ ID NO: 637 - protein, 17 aa, Artificial Sequence
  source: /note=“Description of Artificial Sequence: Synthetic peptide”
LNWYQHKPGKAPKPLVF

SEQ ID NO: 638 - protein, 36 aa, Artificial Sequence
  source: /note=“Description of Artificial Sequence: Synthetic polypeptide”
NLETGVPPRFSATGSGTDFSLTITSLQPEDFATYYC

SEQ ID NO: 639 - protein, 36 aa, Artificial Sequence
  source: /note=“Description of Artificial Sequence: Synthetic polypeptide”
NLETGVPSRFSGSGSGTDFTFTISSLQPEDIATYYC

SEQ ID NO: 640 - protein, 36 aa, Artificial Sequence
  source: /note=“Description of Artificial Sequence: Synthetic polypeptide”
NLETGVPSRFSASGSGTDFTLTISSLQPEDFATYYC

SEQ ID NO: 641 - protein, 10 aa, Artificial Sequence
  source: /note=“Description of Artificial Sequence: Synthetic peptide”
FGQGTRLDIK

SEQ ID NO: 642 - protein, 10 aa, Artificial Sequence
  source: /note=“Description of Artificial Sequence: Synthetic peptide”
FGQGTKVEIK

SEQ ID NO: 643 - protein, 8 aa, Artificial Sequence
  source: /note=“Description of Artificial Sequence: Synthetic peptide”
GFTFRDYA

SEQ ID NO: 644 - protein, 8 aa, Artificial Sequence
  source: /note=“Description of Artificial Sequence: Synthetic peptide”
ISGSGGNT

SEQ ID NO: 645 - protein, 8 aa, Artificial Sequence
  source: /note=“Description of Artificial Sequence: Synthetic peptide”
ISGSGGST

SEQ ID NO: 646 - protein, 15 aa, Artificial Sequence
  source: /note=“Description of Artificial Sequence: Synthetic peptide”
ARGEYSSGWPSWFDP

SEQ ID NO: 647 - protein, 25 aa, Artificial Sequence
  source: /note=“Description of Artificial Sequence: Synthetic peptide”
EVQLVESGGGLEQPGGSLRLSCAGS

SEQ ID NO: 648 - protein, 25 aa, Artificial Sequence
  source: /note=“Description of Artificial Sequence: Synthetic peptide”
EVQLVESGGGLEQPGGSLRLSCAAS

SEQ ID NO: 649 - protein, 17 aa, Artificial Sequence
  source: /note=“Description of Artificial Sequence: Synthetic peptide”
MTWVRQAPGKGLEWVSS

SEQ ID NO: 650 - protein, 17 aa, Artificial Sequence
  source: /note=“Description of Artificial Sequence: Synthetic peptide”
MTWVRQAPGKGLEWVSA

SEQ ID NO: 651 - protein, 38 aa, Artificial Sequence
  source: /note=“Description of Artificial Sequence: Synthetic polypeptide”
YYADSVKGRFTISRDNSKNTLYLQMNSLRAEDTAIYYC

SEQ ID NO: 652 - protein, 6 aa, Artificial Sequence
  source: /note=“Description of Artificial Sequence: Synthetic peptide”
RTVGTF

SEQ ID NO: 653 - protein, 3 aa, Artificial Sequence
  source: /note=“Description of Artificial Sequence: Synthetic peptide”
GVS

SEQ ID NO: 654 - protein, 3 aa, Artificial Sequence
  source: /note=“Description of Artificial Sequence: Synthetic peptide”
HTS

SEQ ID NO: 655 - protein, 9 aa, Artificial Sequence
  source: /note=“Description of Artificial Sequence: Synthetic peptide”
QQSHDVPRT

SEQ ID NO: 656 - protein, 26 aa, Artificial Sequence
  source: /note=“Description of Artificial Sequence: Synthetic peptide”
EIVLTQSPSSLSASVGDRVTISCRAS

SEQ ID NO: 657 - protein, 26 aa, Artificial Sequence [sequence omitted]

SEQ ID NO: 658 - protein, 17 aa, Artificial Sequence
  source: /note=“Description of Artificial Sequence: Synthetic peptide”
LNWYQQRPGKPPQALIL

SEQ ID NO: 659 - protein, 17 aa, Artificial Sequence
  source: /note=“Description of Artificial Sequence: Synthetic peptide”
LNWYQQKPGKAPKALIL

SEQ ID NO: 660 - protein, 36 aa, Artificial Sequence
  source: /note=“Description of Artificial Sequence: Synthetic polypeptide”
ILEGGVSSRFSGAGSGTEFSLTISSLQPEDIGTYYC

SEQ ID NO: 661 - protein, 36 aa, Artificial Sequence
  source: /note=“Description of Artificial Sequence: Synthetic polypeptide”
ILEGGVPSRFSGSGSGTDFTLTISSLQLEDFATYYC

SEQ ID NO: 662 - protein, 36 aa, Artificial Sequence
  source: /note=“Description of Artificial Sequence: Synthetic polypeptide”
ILEGGVPSRFSGSGSGTDFTLTISSLQPEDIGTYYC

SEQ ID NO: 663 - protein, 36 aa, Artificial Sequence
  source: /note=“Description of Artificial Sequence: Synthetic polypeptide”
ILEGGVPSRFSGSGSGTDFTLTISSLQPEDIATYYC

SEQ ID NO: 664 - protein, 10 aa, Artificial Sequence
  source: /note=“Description of Artificial Sequence: Synthetic peptide”
FGQGTKVDLR

SEQ ID NO: 665 - protein, 10 aa, Artificial Sequence
  source: /note=“Description of Artificial Sequence: Synthetic peptide”
FGQGTKVEIK

SEQ ID NO: 666-668 - skipped (3 entries)

SEQ ID NO: 669 - protein, 120 aa, Artificial Sequence
  source: /note=“Description of Artificial Sequence: Synthetic polypeptide”
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGSGWGYAFDIWGQGTMVTVSS

SEQ ID NO: 670 - protein, 120 aa, Artificial Sequence
  source: /note=“Description of Artificial Sequence: Synthetic polypeptide”
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGSGWGYAFDIWGQGTLVTVSS

SEQ ID NO: 671 - protein, 120 aa, Artificial Sequence
  source: /note=“Description of Artificial Sequence: Synthetic polypeptide”
QVQLVESGGGVVQPGRSLRLSCAASGFTFSTYGMHWVRQAPGKGLEWVAVIWDGGKYKYYGDSVKGRFTISRDNSKNTLYLQMNSLRAEDTAVYYCARDGSGWGYAFDIWGQGTLVTVSS

SEQ ID NO: 672 - protein, 120 aa, Artificial Sequence
  source: /note=“Description of Artificial Sequence: Synthetic polypeptide”
QVQLVESGGGVVQPGRSLRLSCAASGFTFSTYGMHWVRQAPGKGLEWVAVIWDGGSYKYYGDSVKGRFTISRDNSKNTLYLQMNSLRAEDTAVYYCARDGSGWGYAFDIWGQGTLVTVSS

SEQ ID NO: 673 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFDAANLETGVPPRFSATGSGTDFSLTITSLQPEDFATYYCQQYDSFPYTFGQGTRLDIK

SEQ ID NO: 674 - protein, 107 aa, Artificial Sequence
  source: /note=“Description of Artificial Sequence: Synthetic polypeptide”
DIQLTQSPSSLSASVGDRVTITCQASQDISKPLNWYQHKPGKAPNPLVFDAANLETGVPPRFSATGSGTDFSLTITSLQPEDFATYYCQQYDSFPYTFGQGTRLDIK

SEQ ID NO: 675 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFRSPNLETGVPPRFSATGSGTDFSLTITSLQPEDFATYYCQQYDSFPYTFGQGTRLDIK

SEQ ID NO: 676 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFGSPNLETGVPPRFSATGSGTDFSLTITSLQPEDFATYYCQQYDSFPYTFGQGTRLDIK

SEQ ID NO: 677 - protein, 107 aa, Artificial Sequence
  source: /note=“Description of Artificial Sequence: Synthetic polypeptide”
DIQLTQSPSSLSASVGDRVTITCQASQDITKGLNWYQHKPGKAPNPLVFDAANLETGVPPRFSATGSGTDFSLTITSLQPEDFATYYCQQYDSFPYTFGQGTRLDIK

SEQ ID NO: 678 - protein, 107 aa, Artificial Sequence
  source: /note=“Description of Artificial Sequence: Synthetic polypeptide”
DIQLTQSPSSLSASVGDRVTITCQASQDIGPWLNWYQHKPGKAPNPLVFDAANLETGVPPRFSATGSGTDFSLTITSLQPEDFATYYCQQYDSFPYTFGQGTRLDIK

SEQ ID NO: 679 - protein, 107 aa, Artificial Sequence
  source: /note=“Description of Artificial Sequence: Synthetic polypeptide”
DIQLTQSPSSLSASVGDRVTITCQASQDIEWWLNWYQHKPGKAPNPLVFDAANLETGVPPRFSATGSGTDFSLTITSLQPEDFATYYCQQYDSFPYTFGQGTRLDIK

SEQ ID NO: 680 - protein, 107 aa, Artificial Sequence
  source: /note=“Description of Artificial Sequence: Synthetic polypeptide”
DIQLTQSPSSLSASVGDRVTITCQASQDIGAYLNWYQHKPGKAPNPLVFDAANLETGVPPRFSATGSGTDFSLTITSLQPEDFATYYCQQYDSFPYTFGQGTRLDIK

SEQ ID NO: 681 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFARDNLETGVPPRFSATGSGTDFSLTITSLQPEDFATYYCQQYDSFPYTFGQGTRLDIK

SEQ ID NO: 682 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFTSANLETGVPPRFSATGSGTDFSLTITSLQPEDFATYYCQQYDSFPYTFGQGTRLDIK

SEQ ID NO: 683 - protein, 107 aa, Artificial Sequence
  source: /note=“Description of Artificial Sequence: Synthetic polypeptide”
DIQLTQSPSSLSASVGDRVTITCQASQDIEWALNWYQHKPGKAPNPLVFDAANLETGVPPRFSATGSGTDFSLTITSLQPEDFATYYCQQYDSFPYTFGQGTRLDIK

SEQ ID NO: 684 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFGADNLETGVPPRFSATGSGTDFSLTITSLQPEDFATYYCQQYDSFPYTFGQGTRLDIK

SEQ ID NO: 685 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFDAPNLETGVPPRFSATGSGTDFSLTITSLQPEDFATYYCQQYDSFPYTFGQGTRLDIK

SEQ ID NO: 686 - protein, 107 aa, Artificial Sequence
  source: /note=“Description of Artificial Sequence: Synthetic polypeptide”
DIQLTQSPSSLSASVGDRVTITCQASQDISEYLNWYQHKPGKAPNPLVFSQPNLETGVPPRFSATGSGTDFSLTITSLQPEDFATYYCQQYDSFPYTFGQGTRLDIK

SEQ ID NO: 687 - protein, 107 aa, Artificial Sequence
  source: /note=“Description of Artificial Sequence: Synthetic polypeptide”
DIQLTQSPSSLSASVGDRVTITCQASQDIMKTLNWYQHKPGKAPNPLVFDAANLETGVPPRFSATGSGTDFSLTITSLQPEDFATYYCQQYDSFPYTFGQGTRLDIK

SEQ ID NO: 688 - protein, 107 aa, Artificial Sequence
  source: /note=“Description of Artificial Sequence: Synthetic polypeptide”
DIQMTQSPSSLSASVGDRVTITCQASQDIGPWLNWYQQKPGKAPKLLIYGSPNLETGVPSRFSGSGSGTDFTFTISSLQPEDIATYYCQQYDSFPYTFGQGTKVEIK

SEQ ID NO: 689 - protein, 107 aa, Artificial Sequence
  source: /note=“Description of Artificial Sequence: Synthetic polypeptide”
DIQMTQSPSSLSASVGDRVTITCQASQDIGPWLNWYQQKPGKAPKLLIYDAPNLETGVPSRFSGSGSGTDFTFTISSLQPEDIATYYCQQYDSFPYTFGQGTKVEIK

SEQ ID NO: 690 - protein, 107 aa, Artificial Sequence
  source: /note=“Description of Artificial Sequence: Synthetic polypeptide”
DIQMTQSPSSLSASVGDRVTITCQASQDISPYLNWYQQKPGKAPKLLIYDAPNLETGVPSRFSGSGSGTDFTFTISSLQPEDIATYYCQQYDSFPYTFGQGTKVEIK

SEQ ID NO: 691 - protein, 107 aa, Artificial Sequence
  source: /note=“Description of Artificial Sequence: Synthetic polypeptide”
DIQLTQSPSSLSASVGDRVTITCQASQDIEWWLNWYQQKPGKAPKLLIYGSPNLETGVPSRFSGSGSGTDFTFTISSLQPEDIATYYCQQYDSFPYTFGQGTKVEIK

SEQ ID NO: 692 - protein, 107 aa, Artificial Sequence
  source: /note=“Description of Artificial Sequence: Synthetic polypeptide”
DIQLTQSPSSLSASVGDRVTITCQASQDIGPWLNWYQHKPGKAPKPLIYGSPNLETGVPSRFSGSGSGTDFTFTISSLQPEDIATYYCQQYDSFPYTFGQGTKVEIK

SEQ ID NO: 693 - protein, 107 aa, Artificial Sequence
  source: /note=“Description of Artificial Sequence: Synthetic polypeptide”
DIQLTQSPSSLSASVGDRVTITCQASQDISPWLNWYQHKPGKAPKPLIYGSPNLETGVPSRFSGSGSGTDFTFTISSLQPEDIATYYCQQYDSFPYTFGQGTKVEIK

SEQ ID NO: 694 - protein, 107 aa, Artificial Sequence
  source: /note=“Description of Artificial Sequence: Synthetic polypeptide”
DIQLTQSPSSLSASVGDRVTITCQASQDIGPWLNWYQHKPGKAPKPLVFGSPNLETGVPSRFSASGSGTDFTLTISSLQPEDFATYYCQQYDSFPYTFGQGTKVEIK

SEQ ID NO: 695 - protein, 107 aa, Artificial Sequence
  source: /note=“Description of Artificial Sequence: Synthetic polypeptide”
DIQLTQSPSSLSASVGDRVTITCQASQDIGPYLNWYQHKPGKAPKPLVFGSPNLETGVPSRFSASGSGTDFTLTISSLQPEDFATYYCQQYDSFPYTFGQGTKVEIK

SEQ ID NO: 696 - protein, 107 aa, Artificial Sequence
  source: /note=“Description of Artificial Sequence: Synthetic polypeptide”
DIQLTQSPSSLSASVGDRVTITCQASQDISPWLNWYQHKPGKAPKPLVFGSPNLETGVPSRFSASGSGTDFTLTISSLQPEDFATYYCQQYDSFPYTFGQGTKVEIK

SEQ ID NO: 697 - protein, 107 aa, Artificial Sequence
  source: /note=“Description of Artificial Sequence: Synthetic polypeptide”
DIQLTQSPSSLSASVGDRVTITCQASQDISPYLNWYQHKPGKAPKPLVFGSPNLETGVPSRFSASGSGTDFTLTISSLQPEDFATYYCQQYDSFPYTFGQGTKVEIK

SEQ ID NO: 698 - protein, 107 aa, Artificial Sequence
  source: /note=“Description of Artificial Sequence: Synthetic polypeptide”
DIQLTQSPSSLSASVGDRVTITCQASQDISYNLNWYQHKPGKAPKPLVFGSPNLETGVPSRFSASGSGTDFTLTISSLQPEDFATYYCQQYDSFPYTFGQGTKVEIK

SEQ ID NO: 699 - protein, 107 aa, Artificial Sequence
  source: /note=“Description of Artificial Sequence: Synthetic polypeptide”
DIQLTQSPSSLSASVGDRVTITCQASQDIEWWLNWYQHKPGKAPKPLIYGSPNLETGVPSRFSGSGSGTDFTFTISSLQPEDIATYYCQQYDSFPYTFGQGTKVEIK

SEQ ID NO: 700 - protein, 107 aa, Artificial Sequence
  source: /note=“Description of Artificial Sequence: Synthetic polypeptide”
DIQLTQSPSSLSASVGDRVTITCQASQDIGPWLNWYQHKPGKAPKPLVFDAPNLETGVPSRFSASGSGTDFTLTISSLQPEDFATYYCQQYDSFPYTFGQGTKVEIK

SEQ ID NO: 701 - protein, 107 aa, Artificial Sequence
  source: /note=“Description of Artificial Sequence: Synthetic polypeptide”
DIQLTQSPSSLSASVGDRVTITCQASQDIGPWLNWYQHKPGKAPKPLVFGADNLETGVPSRFSASGSGTDFTLTISSLQPEDFATYYCQQYDSFPYTFGQGTKVEIK

SEQ ID NO: 702 - protein, 107 aa, Artificial Sequence
  source: /note=“Description of Artificial Sequence: Synthetic polypeptide”
DIQLTQSPSSLSASVGDRVTITCQASQDIGPWLNWYQHKPGKAPKPLVFRSPNLETGVPSRFSASGSGTDFTLTISSLQPEDFATYYCQQYDSFPYTFGQGTKVEIK

SEQ ID NO: 703 - protein, 122 aa, Artificial Sequence
  source: /note=“Description of Artificial Sequence: Synthetic polypeptide”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAIYYCARGEYSSGWPSWFDPWGQGTLVTVSS

SEQ ID NO: 704 - protein, 122 aa, Artificial Sequence
  source: /note=“Description of Artificial Sequence: Synthetic polypeptide”
EVQLVESGGGLEQPGGSLRLSCAGSGFTFRDYAMTWVRQAPGKGLEWVSSISGSGGSTYYADSVKGRFTISRDNSKNTLYLQMNSLRAEDTAIYYCARGEYSSGWPSWFDPWGQGTLVTVSS

SEQ ID NO: 705 - protein, 122 aa, Artificial Sequence
  source: /note=“Description of Artificial Sequence: Synthetic polypeptide”
EVQLVESGGGLEQPGGSLRLSCAASGFTFRDYAMTWVRQAPGKGLEWVSSISGSGGSTYYADSVKGRFTISRDNSKNTLYLQMNSLRAEDTAIYYCARGEYSSGWPSWFDPWGQGTLVTVSS

SEQ ID NO: 706 - protein, 122 aa, Artificial Sequence
  source: /note=“Description of Artificial Sequence: Synthetic polypeptide”
EVQLVESGGGLEQPGGSLRLSCAASGFTFRDYAMTWVRQAPGKGLEWVSAISGSGGSTYYADSVKGRFTISRDNSKNTLYLQMNSLRAEDTAIYYCARGEYSSGWPSWFDPWGQGTLVTVSS

SEQ ID NO: 707 - protein, 107 aa, Artificial Sequence
  source: /note=“Description of Artificial Sequence: Synthetic polypeptide”
EIVLTQSPSSLSASVGDRVTISCRASRTVGTFLNWYQQRPGKPPQALILGVSILEGGVSSRFSGAGSGTEFSLTISSLQPEDIGTYYCQQSHDVPRTFGQGTKVDLR

SEQ ID NO: 708 - protein, 107 aa, Artificial Sequence
  source: /note=“Description of Artificial Sequence: Synthetic polypeptide”
EIVLTQSPSSLSASVGDRVTISCRASRTVGTFLNWYQQRPGKPPQALILHTSILEGGVSSRFSGAGSGTEFSLTISSLQPEDIGTYYCQQSHDVPRTFGQGTKVDLR

SEQ ID NO: 709 - protein, 107 aa, Artificial Sequence
  source: /note=“Description of Artificial Sequence: Synthetic polypeptide”
DIQMTQSPSSLSASVGDRVTITCRASRTVGTFLNWYQQKPGKAPKALILGVSILEGGVPSRFSGSGSGTDFTLTISSLQLEDFATYYCQQSHDVPRTFGQGTKVEIK

SEQ ID NO: 710 - protein, 107 aa, Artificial Sequence
  source: /note=“Description of Artificial Sequence: Synthetic polypeptide”
DIQMTQSPSSLSASVGDRVTITCRASRTVGTFLNWYQQKPGKAPKALILGVSILEGGVPSRFSGSGSGTDFTLTISSLQPEDIGTYYCQQSHDVPRTFGQGTKVEIK

SEQ ID NO: 711 - protein, 107 aa, Artificial Sequence
  source: /note=“Description of Artificial Sequence: Synthetic polypeptide”
DIQMTQSPSSLSASVGDRVTITCRASRTVGTFLNWYQQKPGKAPKALILGVSILEGGVPSRFSGSGSGTDFTLTISSLQPEDIATYYCQQSHDVPRTFGQGTKVEIK

SEQ ID NO: 712 - protein, 107 aa, Artificial Sequence
  source: /note=“Description of Artificial Sequence: Synthetic polypeptide”
DIQMTQSPSSLSASVGDRVTITCRASRTVGTFLNWYQQKPGKAPKALILHTSILEGGVPSRFSGSGSGTDFTLTISSLQPEDIGTYYCQQSHDVPRTFGQGTKVEIK

SEQ ID NO: 713 - protein, 107 aa, Artificial Sequence
  source: /note=“Description of Artificial Sequence: Synthetic polypeptide”
DIQMTQSPSSLSASVGDRVTITCRASRTVGTFLNWYQQKPGKAPKALILHTSILEGGVPSRFSGSGSGTDFTLTISSLQPEDIATYYCQQSHDVPRTFGQGTKVEIK

SEQ ID NO: 714 - protein, 8 aa, Artificial Sequence
  source: /note=“Description of Artificial Sequence: Synthetic peptide”
  VARIANT (1)..(1): /replace=“E” or “F” or “G” or “H” or “I” or “K” or “L” or “M” or “N” or “P” or “Q” or “R” or “T” or “V” or “W” or “Y”
  VARIANT (2)..(2): /replace=“C” or “D” or “E” or “F” or “G” or “H” or “K” or “M” or “N” or “P” or “Q” or “R” or “T” or “W”
  VARIANT (3)..(3): /replace=“C” or “D” or “E” or “F” or “G” or “H” or “K” or “L” or “M” or “P” or “Q” or “R” or “T” or “V” or “W” or “Y”
  VARIANT (4)..(4): /replace=“C” or “D” or “E” or “F” or “G” or “H” or “K” or “M” or “N” or “P” or “Q” or “R” or “T” or “W”
  VARIANT (5)..(5): /replace=“C” or “D” or “E” or “G” or “H” or “I” or “K” or “L” or “M” or “N” or “Q” or “R” or “S” or “V” or “W”
  VARIANT (6)..(6): /replace=“D” or “E” or “F” or “G” or “H” or “I” or “L” or “N” or “P” or “Q” or “T” or “V” or “W” or “Y”
  VARIANT (7)..(7): /replace=“D” or “E” or “F” or “G” or “I” or “K” or “L” or “M” or “P” or “Q” or “R” or “S” or “T” or “V” or “W” or “Y”
  VARIANT (8)..(8): /replace=“F” or “G” or “H” or “I” or “K” or “L” or “M” or “N” or “P” or “Q” or “R” or “T” or “W” or “Y”
  SITE (1)..(8): /note=“Variant residues given in the sequence have no preference with respect to those in the annotations for variant positions”
AAAAACAE

SEQ ID NO: 715 - protein, 8 aa, Artificial Sequence
  source: /note=“Description of Artificial Sequence: Synthetic peptide”
  VARIANT (1)..(1): /replace=“C” or “D” or “E” or “G” or “H” or “I” or “K” or “L” or “M” or “P” or “Q” or “R” or “W” or “Y”
  VARIANT (2)..(2): /replace=“D” or “E” or “F” or “H” or “I” or “K” or “M” or “N” or “P” or “Q” or “R” or “T” or “V” or “W” or “Y”
  VARIANT (3)..(3): /replace=“D”
  VARIANT (4)..(4): /replace=“D” or “E” or “F” or “G”
  VARIANT (5)..(5): /replace=“E” or “F” or “G” or “H” or “I” or “K” or “L” or “M” or “N” or “P” or “Q” or “T” or “W” or “Y”
  VARIANT (6)..(6): /replace=“F” or “H” or “K” or “P” or “R” or “S” or “T” or “W” or “Y”
  VARIANT (7)..(7): /replace=“D” or “E” or “F” or “G” or “I” or “K” or “L” or “M” or “P” or “Q” or “R” or “T” or “V” or “W” or “Y”
  VARIANT (8)..(8): /replace=“C” or “D” or “F” or “G” or “H” or “I” or “K” or “L” or “P” or “Q” or “S” or “V” or “W” or “Y”
  SITE (1)..(8): /note=“Variant residues given in the sequence have no preference with respect to those in the annotations for variant positions”
  source: /note=“See specification as filed for detailed description of substitutions and preferred embodiments”
AACADCAA

SEQ ID NO: 716 - protein, 13 aa, Artificial Sequence
  source: /note=“Description of Artificial Sequence: Synthetic peptide”
  VARIANT (1)..(1): /replace=“C” or “D” or “F” or “G” or “H” or “I” or “K” or “L” or “M” or “N” or “Q” or “R” or “W” or “Y”
  VARIANT (2)..(2): /replace=“C” or “D” or “E” or “F” or “H” or “L” or “M” or “N” or “P” or “Q” or “R” or “V” or “W” or “Y”
  VARIANT (3)..(3): /replace=“D” or “F” or “I” or “Q”
  VARIANT (4)..(4): /replace=“F” or “G” or “H” or “I” or “K” or “L” or “M” or “N” or “P” or “Q”
  VARIANT (5)..(5): /replace=“D” or “E” or “F” or “H” or “I” or “K” or “L” or “M” or “P” or “Q” or “S” or “T” or “V” or “W” or “Y”
  VARIANT (6)..(6): /replace=“E” or “F” or “G” or “H” or “I” or “K” or “L” or “M” or “N” or “P” or “Q” or “R” or “T” or “W” or “Y”
  VARIANT (7)..(7): /replace=“D” or “E” or “F” or “H” or “I” or “M” or “N” or “P” or “Q” or “S” or “T” or “V” or “W” or “Y”
  VARIANT (8)..(8): /replace=“C” or “D” or “F” or “G” or “H” or “I” or “K” or “L” or “M” or “N” or “P” or “Q” or “S” or “T” or “W” or “Y”
  VARIANT (9)..(9): /replace=“E” or “G” or “I” or “K” or “L” or “M” or “P” or “Q” or “R” or “T” or “V” or “W” or “Y”
  VARIANT (10)..(10): /replace=“C” or “E” or “F” or “H” or “I” or “K” or “L” or “M” or “N” or “Q” or “R”
  VARIANT (11)..(11): /replace=“F” or “G” or “H” or “M” or “N” or “P” or “R” or “T” or “W”
  VARIANT (12)..(12): /replace=“D” or “F” or “K” or “L” or “M” or “P” or “Q” or “R” or “W”
  VARIANT (13)..(13): /replace=“H” or “I” or “K” or “M” or “P” or “Q” or “R” or “W” or “Y”
  SITE (1)..(13): /note=“Variant residues given in the sequence have no preference with respect to those in the annotations for variant positions”
  source: /note=“See specification as filed for detailed description of substitutions and preferred embodiments”
AACEAAAAAADCG

SEQ ID NO: 717 - protein, 6 aa, Artificial Sequence
  source: /note=“Description of Artificial Sequence: Synthetic peptide”
  VARIANT (1)..(1): /replace=“C” or “D” or “E” or “F” or “G” or “I” or “K” or “L” or “M” or “N” or “Q” or “S” or “T” or “V” or “W” or “Y”
  VARIANT (2)..(2): /replace=“C” or “D” or “E” or “F” or “H” or “I” or “K” or “L” or “M” or “N” or “P” or “Q” or “R” or “T” or “V” or “W”
  VARIANT (3)..(3): /replace=“C” or “D” or “E” or “F” or “G” or “H” or “I” or “K” or “M” or “P” or “Q” or “R” or “V” or “W” or “Y”
  VARIANT (4)..(4): /replace=“D” or “E” or “F” or “G” or “K” or “L” or “M” or “R” or “S” or “T” or “V” or “W” or “Y”
  VARIANT (5)..(5): /replace=“D” or “E” or “F” or “G” or “H” or “I” or “L” or “M” or “P” or “Q” or “S” or “T” or “V” or “W” or “Y”
  VARIANT (6)..(6): /replace=“C” or “F” or “G” or “H” or “I” or “Q” or “S” or “T” or “W” or “Y”
  SITE (1)..(6): /note=“Variant residues given in the sequence have no preference with respect to those in the annotations for variant positions”
  source: /note=“See specification as filed for detailed description of substitutions and preferred embodiments”
AAACCA

SEQ ID NO: 718 - protein, 3 aa, Artificial Sequence
  source: /note=“Description of Artificial Sequence: Synthetic peptide”
  VARIANT (1)..(1): /replace=“C” or “D” or “F” or “G” or “H” or “N” or “R” or “S”
  VARIANT (2)..(2): /replace=“C” or “D” or “I” or “K” or “S” or “T”
  VARIANT (3)..(3): /replace=“C” or “D” or “E” or “F” or “H” or “I” or “N” or “P” or “S” or “T” or “V” or “W”
  SITE (1)..(3): /note=“Variant residues given in the sequence have no preference with respect to those in the annotations for variant positions”
  source: /note=“See specification as filed for detailed description of substitutions and preferred embodiments”
AAA

SEQ ID NO: 719 - protein, 9 aa, Artificial Sequence
  source: /note=“Description of Artificial Sequence: Synthetic peptide”
  VARIANT (1)..(1): /replace=“C” or “D” or “E” or “F” or “G” or “I” or “M” or “N” or “P” or “Q” or “S” or “T” or “V” or “W” or “Y”
  VARIANT (2)..(2): /replace=“C” or “D” or “E” or “F” or “G” or “I” or “M” or “N” or “P” or “Q” or “S” or “T” or “V” or “W” or “Y”
  VARIANT (3)..(3): /replace=“C” or “D” or “E” or “G” or “I” or “K” or “L” or “M” or “N” or “P” or “Q” or “R” or “T” or “V” or “W” or “Y”
  VARIANT (4)..(4): /replace=“E” or “F” or “P” or “Q” or “Y”
  VARIANT (5)..(5): /replace=“K” or “L” or “M” or “N” or “P” or “Q” or “R” or “S”
  VARIANT (6)..(6): /replace=“D” or “E” or “F” or “H” or “I” or “L” or “M” or “N” or “P” or “Q” or “S” or “T” or “V” or “Y”
  VARIANT (7)..(7): /replace=“C” or “D” or “E” or “F” or “G” or “I” or “K” or “L” or “M” or “N” or “P” or “Q” or “R” or “V” or “W” or “Y”
  VARIANT (8)..(8): /replace=“E” or “F” or “G” or “I” or “K” or “M” or “N” or “P” or “Q” or “R” or “T” or “V” or “W” or “Y”
  VARIANT (9)..(9): /replace=“D” or “E” or “F” or “G” or “H” or “I” or “K” or “L” or “M” or “N” or “Q” or “R” or “T” or “V” or “W” or “Y”
  SITE (1)..(9): /note=“Variant residues given in the sequence have no preference with respect to those in the annotations for variant positions”
AAADGCAAC

SEQ ID NO: 720 - protein, 8 aa, Artificial Sequence
  source: /note=“Description of Artificial Sequence: Synthetic peptide”
  VARIANT (1)..(1): /replace=“C” or “D” or “E” or “F” or “G” or “H” or “I” or “K” or “L” or “M” or “N” or “P” or “Q” or “R” or “S” or “T” or “V” or “W” or “Y”
  VARIANT (2)..(2): /replace=“C” or “D” or “E” or “F” or “G” or “H” or “K” or “L” or “M” or “N” or “P” or “Q” or “R” or “S” or “T” or “V” or “W”
  VARIANT (3)..(3): /replace=“C” or “D” or “E” or “F” or “G” or “H” or “I” or “K” or “L” or “M” or “N” or “P” or “Q” or “R” or “T” or “V” or “W” or “Y”
  VARIANT (4)..(4): /replace=“C” or “D” or “E” or “F” or “G” or “H” or “I” or “K” or “M” or “N” or “P” or “Q” or “R” or “S” or “T” or “V” or “W” or “Y”
  VARIANT (5)..(5): /replace=“K” or “L” or “M” or “P” or “Q” or “V” or “W” or “Y”
  VARIANT (6)..(6): /replace=“G” or “H” or “I” or “K” or “L” or “M” or “P” or “Q” or “T” or “V” or “W” or “Y”
  VARIANT (7)..(7): /replace=“C” or “D” or “E” or “F” or “G” or “H” or “I” or “K” or “M” or “N” or “P” or “Q” or “R” or “S” or “T” or “V” or “W” or “Y”
  VARIANT (8)..(8): /replace=“C” or “D” or “E” or “F” or “H” or “K” or “L” or “M” or “N” or “P” or “Q” or “R” or “T” or “V”
  SITE (1)..(8): /note=“Variant residues given in the sequence have no preference with respect to those in the annotations for variant positions”
  source: /note=“See specification as filed for detailed description of substitutions and preferred embodiments”
AAAAIFAA

SEQ ID NO: 721 - protein, 8 aa, Artificial Sequence
  source: /note=“Description of Artificial Sequence: Synthetic peptide”
  VARIANT (1)..(1): /replace=“C” or “D” or “E” or “G” or “H” or “I” or “K” or “L” or “M” or “N” or “P” or “Q” or “R” or “S” or “T” or “V” or “W” or “Y”
  VARIANT (2)..(2): /replace=“C” or “E” or “F” or “G” or “H” or “I” or “K” or “L” or “M” or “P” or “Q” or “R” or “S” or “V” or “Y”
  VARIANT (3)..(3): /replace=“D” or “E” or “F” or “G” or “H” or “I” or “K” or “L” or “M” or “N” or “Q” or “R” or “S” or “V”
  VARIANT (4)..(4): /replace=“C” or “F” or “H” or “K” or “L” or “M” or “P” or “Q” or “R” or “S” or “T” or “V” or “W”
  VARIANT (5)..(5): /replace=“C” or “E” or “F” or “G” or “H” or “I” or “K” or “L” or “M” or “P” or “Q” or “R” or “V” or “W”
  VARIANT (6)..(6): /replace=“C” or “D” or “E” or “F” or “G” or “H” or “I” or “K” or “L” or “M” or “N” or “P” or “Q” or “R” or “T” or “V” or “W” or “Y”
  VARIANT (7)..(7): /replace=“C” or “F” or “G” or “I” or “K” or “L” or “M” or “N” or “P” or “Q” or “R” or “S” or “T” or “V” or “W” or “Y”
  VARIANT (8)..(8): /replace=“D” or “E” or “F” or “G” or “H” or “K” or “L” or “M” or “N” or “P” or “Q” or “S” or “T” or “V” or “W” or “Y”
  SITE (1)..(8): /note=“Variant residues given in the sequence have no preference with respect to those in the annotations for variant positions”
  source: /note=“See specification as filed for detailed description of substitutions and preferred embodiments”
AACAAAAC

SEQ ID NO: 722 - protein, 15 aa, Artificial Sequence
  source: /note=“Description of Artificial Sequence: Synthetic peptide”
  VARIANT (1)..(1): /replace=“C” or “D” or “E” or “F” or “G” or “H” or “I” or “K” or “L” or “M” or “N” or “P” or “Q” or “R” or “S” or “T” or “V” or “W” or “Y”
  VARIANT (2)..(2): /replace=“G” or “H” or “I” or “T”
  VARIANT (3)..(3): /replace=“C” or “D” or “F” or “G” or “H” or “I” or “K” or “L” or “M” or “N” or “P” or “Q” or “R” or “S” or “T” or “V” or “W” or “Y”
  VARIANT (4)..(4): /replace=“E” or “F” or “H” or “N” or “Q” or “R” or “S” or “T” or “V” or “W” or “Y”
  VARIANT (5)..(5): /replace=“H” or “I” or “L” or “M” or “N” or “Q” or “Y”
  VARIANT (6)..(6): /replace=“C” or “D” or “F” or “G” or “H” or “K” or “M” or “N” or “P” or “Q” or “R” or “S” or “T” or “V” or “Y”
  VARIANT (7)..(7): /replace=“C” or “E” or “F” or “H” or “K” or “M” or “N” or “P” or “Q” or “S” or “T” or “W” or “Y”
  VARIANT (8)..(8): /replace=“C” or “D” or “E” or “F” or “G” or “H” or “I” or “K” or “L” or “M” or “N” or “P” or “Q” or “S” or “T” or “V” or “W”
  VARIANT (9)..(9): /replace=“C” or “D” or “E” or “F” or “H” or “I” or “K” or “L” or “N” or “Q” or “R” or “S” or “V” or “W” or “Y”
  VARIANT (10)..(10): /replace=“C” or “D” or “E” or “F” or “H” or “I” or “K” or “L” or “M” or “N” or “P” or “Q” or “R” or “S” or “V” or “W” or “Y”
  VARIANT (11)..(11): /replace=“C” or “E” or “F” or “H” or “I” or “K” or “L” or “M” or “N” or “P” or “Q” or “S” or “T” or “V” or “W” or “Y”
  VARIANT (12)..(12): /replace=“H” or “I” or “K” or “N” or “P” or “Q” or “R” or “V” or “W” or “Y”
  VARIANT (13)..(13): /replace=“C” or “D” or “E” or “F” or “G” or “H” or “I” or “K” or “L” or “N” or “P” or “Q” or “R” or “S” or “T” or “V” or “W” or “Y”
  VARIANT (14)..(14): /replace=“F” or “G” or “H” or “P” or “Q” or “T”
  VARIANT (15)..(15): /replace=“E” or “F” or “G” or “I” or “K” or “L” or “N” or “P” or “Q” or “R” or “S” or “T”
  SITE (1)..(15): /note=“Variant residues given in the sequence have no preference with respect to those in the annotations for variant positions”
AFADAAAAAAAFADD

SEQ ID NO: 723 - protein, 6 aa, Artificial Sequence
  source: /note=“Description of Artificial Sequence: Synthetic peptide”
  VARIANT (1)..(1): /replace=“C” or “D” or “E” or “F” or “G” or “I” or “K” or “M” or “N” or “R” or “S” or “T” or “V” or “W” or “Y”
  VARIANT (2)..(2): /replace=“D” or “E” or “H” or “K” or “L” or “M” or “Q” or “T” or “W” or “Y”
  VARIANT (3)..(3): /replace=“C” or “D” or “E” or “F” or “G” or “H” or “K” or “M” or “N” or “P” or “Q” or “R” or “T” or “V” or “W” or “Y”
  VARIANT (4)..(4): /replace=“E” or “G” or “I” or “L” or “M” or “P” or “Q” or “V” or “W” or “Y”
  VARIANT (5)..(5): /replace=“M” or “P” or “Q” or “T” or “W”
  VARIANT (6)..(6): /replace=“C” or “E” or “F” or “G” or “I” or “K” or “L” or “M” or “N” or “P” or “Q” or “R” or “T” or “V” or “Y”
  SITE (1)..(6): /note=“Variant residues given in the sequence have no preference with respect to those in the annotations for variant positions”
ACACCA

SEQ ID NO: 724 - protein, 3 aa, Artificial Sequence
  source: /note=“Description of Artificial Sequence: Synthetic peptide”
  VARIANT (1)..(1): /replace=“H” or “I” or “L” or “M” or “N” or “P” or “Q” or “W” or “Y”
  VARIANT (2)..(2): /replace=“D” or “E” or “F” or “G” or “H” or “I” or “K” or “L” or “M” or “N” or “P” or “Q” or “R” or “S” or “T” or “V” or “W” or “Y”
  VARIANT (3)..(3): /replace=“D” or “E” or “G” or “H” or “I” or “K” or “L” or “M” or “P” or “Q” or “R” or “S” or “W” or “Y”
  SITE (1)..(3): /note=“Variant residues given in the sequence have no preference with respect to those in the annotations for variant positions”
  source: /note=“See specification as filed for detailed description of substitutions and preferred embodiments”
CCC

SEQ ID NO: 725 - protein, 9 aa, Artificial Sequence
  source: /note=“Description of Artificial Sequence: Synthetic peptide”
  VARIANT (1)..(1): /replace=“D” or “F” or “G” or “I” or “K” or “M” or “N” or “P” or “Q” or “S” or “T” or “V” or “W” or “Y”
  VARIANT (2)..(2): /replace=“C” or “D” or “F” or “G” or “I” or “L” or “M” or “N” or “P” or “Q” or “R” or “S” or “T” or “V” or “W”
  VARIANT (3)..(3): /replace=“E” or “G” or “K” or “M” or “P” or “S” or “V” or “W”
  VARIANT (4)..(4): /replace=“H” or “L” or “M” or “P” or “Q” or “R” or “V” or “W”
  VARIANT (5)..(5): /replace=“D” or “E” or “F” or “L” or “M” or “P” or “V” or “W”
  VARIANT (6)..(6): /replace=“C” or “E” or “G” or “H” or “K” or “M” or “N” or “P” or “Q” or “R” or “V” or “W”
  VARIANT (7)..(7): /replace=“C” or “D” or “E” or “G” or “H” or “I” or “K” or “M” or “N” or “P” or “R” or “S” or “T” or “V” or “W” or “Y”
  VARIANT (8)..(8): /replace=“C” or “D” or “E” or “G” or “K” or “M” or “N” or “P” or “Q” or “R” or “S” or “W”
  VARIANT (9)..(9): /replace=“D” or “E” or “F” or “G” or “H” or “K” or “L” or “M” or “Q” or “R” or “T” or “V” or “W” or “Y”
  SITE (1)..(9): /note=“Variant residues given in the sequence have no preference with respect to those in the annotations for variant positions”
CACCCAAAC

SEQ ID NO: 726-733 - skipped (8 entries)

SEQ ID NO: 734 - protein, 10 aa, Artificial Sequence
  source: /note=“Description of Artificial Sequence: Synthetic peptide”
  VARIANT (1)..(1): /replace=“E” or “F” or “G” or “H” or “I” or “K” or “L” or “M” or “N” or “P” or “Q” or “R” or “T” or “V” or “W” or “Y”
  VARIANT (2)..(2): /replace=“C” or “D” or “E” or “F” or “G” or “H” or “K” or “M” or “N” or “P” or “Q” or “R” or “T” or “W”
  VARIANT (3)..(3): /replace=“C” or “D” or “E” or “F” or “G” or “H” or “K” or “L” or “M” or “P” or “Q” or “R” or “T” or “V” or “W” or “Y”
  VARIANT (4)..(4): /replace=“C” or “D” or “E” or “F” or “G” or “H” or “K” or “M” or “N” or “P” or “Q” or “R” or “T” or “W”
  VARIANT (5)..(5): /replace=“C” or “D” or “E” or “G” or “H” or “I” or “K” or “L” or “M” or “N” or “Q” or “R” or “S” or “V” or “W”
  VARIANT (6)..(6): /replace=“D” or “E” or “F” or “G” or “H” or “I” or “L” or “N” or “P” or “Q” or “T” or “V” or “W” or “Y”
  VARIANT (7)..(7): /replace=“D” or “E” or “F” or “G” or “I” or “K” or “L” or “M” or “P” or “Q” or “R” or “S” or “T” or “V” or “W” or “Y”
  VARIANT (8)..(8): /replace=“F” or “G” or “H” or “I” or “K” or “L” or “M” or “N” or “P” or “Q” or “R” or “T” or “W” or “Y”
  VARIANT (9)..(9): /replace=“C” or “D” or “E” or “F” or “G” or “H” or “M” or “N” or “P” or “Q” or “R” or “S” or “W” or “Y”
  VARIANT (10)..(10): /replace=“C” or “D” or “E” or “F” or “G” or “H” or “I” or “K” or “M” or “S” or “T” or “V” or “W”
  SITE (1)..(10): /note=“Variant residues given in the sequence have no preference with respect to those in the annotations for variant positions”
AAAAACAEAA

SEQ ID NO: 735 - protein, 17 aa, Artificial Sequence
  source: /note=“Description of Artificial Sequence: Synthetic peptide”
  VARIANT (1)..(1): /replace=“D” or “F” or “H” or “I” or “K” or “N” or “P” or “Q” or “R” or “S” or “T” or “V” or “W” or “Y”
  VARIANT (2)..(2): /replace=“C” or “D” or “E” or “G” or “H” or “I” or “K” or “L” or “M” or “P” or “Q” or “R” or “W” or “Y”
  VARIANT (3)..(3): /replace=“D” or “E” or “F” or “H” or “I” or “K” or “M” or “N” or “P” or “Q” or “R” or “T” or “V” or “W” or “Y”
  VARIANT (4)..(4): /replace=“D”
  VARIANT (5)..(5): /replace=“D” or “E” or “F” or “G”
  VARIANT (6)..(6): /replace=“E” or “F” or “G” or “H” or “I” or “K” or “L” or “M” or “N” or “P” or “Q” or “T” or “W” or “Y”
  VARIANT (7)..(7): /replace=“F” or “H” or “K” or “P” or “R” or “S” or “T” or “W” or “Y”
  VARIANT (8)..(8): /replace=“D” or “E” or “F” or “G” or “I” or “K” or “L” or “M” or “P” or “Q” or “R” or “T” or “V” or “W” or “Y”
  VARIANT (9)..(9): /replace=“C” or “D” or “F” or “G” or “H” or “I” or “K” or “L” or “P” or “Q” or “S” or “V” or “W” or “Y”
  VARIANT (10)..(10): /replace=“G” or “I” or “L” or “M” or “P” or “R” or “V” or “W” or “Y”
  VARIANT (11)..(11): /replace=“E” or “G” or “I” or “K” or “L” or “M” or “P” or “Q” or “R” or “T” or “V” or “W” or “Y”
  VARIANT (12)..(12): /replace=“F” or “G” or “H” or “I” or “K” or “L” or “M” or “N” or “P” or “Q” or “T” or “V” or “W” or “Y”
  VARIANT (13)..(13): /replace=“D” or “F” or “I” or “K” or “L” or “M” or “P” or “Q” or “R” or “S” or “T” or “W”
  VARIANT (14)..(14): /replace=“D” or “E” or “F” or “H” or “K” or “L” or “M” or “P” or “Q” or “S” or “V” or “W” or “Y”
  VARIANT (15)..(15): /replace=“F” or “K” or “M” or “N” or “P” or “Q” or “R” or “S” or “T” or “V” or “W” or “Y”
  VARIANT (16)..(16): /replace=“C” or “D” or “F” or “G” or “H” or “K” or “L” or “M” or “S” or “V” or “W” or “Y”
  VARIANT (17)..(17): /replace=“F” or “G” or “H” or “I” or “K” or “L” or “M” or “N” or “P” or “Q” or “S” or “T” or “W” or “Y”
  SITE (1)..(17): /note=“Variant residues given in the sequence have no preference with respect to those in the annotations for variant positions”
CAACADCAAAAECACAE

SEQ ID NO: 736 - protein, 13 aa, Artificial Sequence
  source: /note=“Description of Artificial Sequence: Synthetic peptide”
  VARIANT (1)..(1): /replace=“C” or “D” or “F” or “G” or “H” or “I” or “K” or “L” or “M” or “N” or “Q” or “R” or “W” or “Y”
  VARIANT (2)..(2): /replace=“C” or “D” or “E” or “F” or “H” or “L” or “M” or “N” or “P” or “Q” or “R” or “V” or “W” or “Y”
  VARIANT (3)..(3): /replace=“D” or “F” or “I” or “Q”
  VARIANT (4)..(4): /replace=“F” or “G” or “H” or “I” or “K” or “L” or “M” or “N” or “P” or “Q”
  VARIANT (5)..(5): /replace=“D” or “E” or “F” or “H” or “I” or “K” or “L” or “M” or “P” or “Q” or “S” or “T” or “V” or “W” or “Y”
  VARIANT (6)..(6): /replace=“E” or “F” or “G” or “H” or “I” or “K” or “L” or “M” or “N” or “P” or “Q” or “R” or “T” or “W” or “Y”
  VARIANT (7)..(7): /replace=“D” or “E” or “F” or “H” or “I” or “M” or “N” or “P” or “Q” or “S” or “T” or “V” or “W” or “Y”
  VARIANT (8)..(8): /replace=“C” or “D” or “F” or “G” or “H” or “I” or “K” or “L” or “M” or “N” or “P” or “Q” or “S” or “T” or “W” or “Y”
  VARIANT (9)..(9): /replace=“E” or “G” or “I” or “K” or “L” or “M” or “P” or “Q” or “R” or “T” or “V” or “W” or “Y”
  VARIANT (10)..(10): /replace=“C” or “E” or “F” or “H” or “I” or “K” or “L” or “M” or “N” or “Q” or “R”
  VARIANT (11)..(11): /replace=“F” or “G” or “H” or “M” or “N” or “P” or “R” or “T” or “W”
  VARIANT (12)..(12): /replace=“D” or “F” or “K” or “L” or “M” or “P” or “Q” or “R” or “W”
  VARIANT (13)..(13): /replace=“H” or “I” or “K” or “M” or “P” or “Q” or “R” or “W” or “Y”
  SITE (1)..(13): /note=“Variant residues given in the sequence have no preference with respect to those in the annotations for variant positions”
AACEAAAAAADCG

SEQ ID NO: 737 - protein, 11 aa, Artificial Sequence
  source: /note=“Description of Artificial Sequence: Synthetic peptide”
  VARIANT (1)..(1): /replace=“C” or “D” or “E” or “F” or “G” or “I” or “L” or “M” or “N” or “P” or “Q” or “S” or “T” or “V” or “W” or “Y”
  VARIANT (2)..(2): /replace=“C” or “D” or “F” or “G” or “H” or “I” or “K” or “L” or “M” or “N” or “P” or “Q” or “R” or “T” or “V” or “W” or “Y”
  VARIANT (3)..(3): /replace=“D” or “E” or “F” or “H” or “K” or “L” or “M” or “N” or “P” or “Q” or “S” or “T” or “V” or “W” or “Y”
  VARIANT (4)..(4): /replace=“C” or “D” or “E” or “F” or “G” or “I” or “K” or “L” or “M” or “N” or “Q” or “S” or “T” or “V” or “W” or “Y”
  VARIANT (5)..(5): /replace=“C” or “D” or “E” or “F” or “H” or “I” or “K” or “L” or “M” or “N” or “P” or “Q” or “R” or “T” or “V” or “W”
  VARIANT (6)..(6): /replace=“C” or “D” or “E” or “F” or “G” or “H” or “I” or “K” or “M” or “P” or “Q” or “R” or “V” or “W” or “Y”
  VARIANT (7)..(7): /replace=“D” or “E” or “F” or “G” or “K” or “L” or “M” or “R” or “S” or “T” or “V” or “W” or “Y”
  VARIANT (8)..(8): /replace=“D” or “E” or “F” or “G” or “H” or “I” or “L” or “M” or “P” or “Q” or “S” or “T” or “V” or “W” or “Y”
  VARIANT (9)..(9): /replace=“C” or “F” or “G” or “H” or “I” or “Q” or “S” or “T” or “W” or “Y”
  VARIANT (10)..(10): /replace=“C” or “D” or “E” or “F” or “G” or “H” or “K” or “L” or “M” or “N” or “P” or “Q” or “R” or “S” or “T” or “W” or “Y”
  VARIANT (11)..(11): /replace=“C” or “D” or “E” or “F” or “G” or “H” or “L” or “M” or “N” or “P” or “Q” or “R” or “S” or “T” or “V” or “W” or “Y”
  SITE (1)..(11): /note=“Variant residues given in the sequence have no preference with respect to those in the annotations for variant positions”
AAAAAACCAAA

SEQ ID NO: 738 - protein, 12 aa, Artificial Sequence
  source: /note=“Description of Artificial Sequence: Synthetic peptide”
  VARIANT (1)..(1): /replace=“C” or “D” or “E” or “F” or “G” or “K” or “L” or “M” or “N” or “P” or “Q” or “R” or “S” or “V” or “W” or “Y”
  VARIANT (2)..(2): /replace=“C” or “D” or “E” or “F” or “G” or “I” or “K” or “M” or “N” or “P” or “Q” or “R” or “S” or “V” or “W” or “Y”
  VARIANT (3)..(3): /replace=“C” or “D” or “E” or “F” or “G” or “H” or “I” or “K” or “L” or “N” or “S” or “T” or “V” or “W” or “Y”
  VARIANT (4)..(4): /replace=“C” or “E” or “H” or “K” or “L” or “M” or “N” or “P” or “Q” or “R” or “S” or “T” or “V” or “W” or “Y”
  VARIANT (5)..(5): /replace=“D” or “E” or “F” or “G” or “H” or “K” or “N” or “P” or “Q” or “R” or “T” or “W”
  VARIANT (6)..(6): /replace=“C” or “D” or “F” or “G” or “H” or “N” or “R” or “S”
  VARIANT (7)..(7): /replace=“C” or “D” or “I” or “K” or “S” or “T”
  VARIANT (8)..(8): /replace=“C” or “D” or “E” or “F” or “H” or “I” or “N” or “P” or “S” or “T” or “V” or “W”
  VARIANT (9)..(9): /replace=“C” or “E” or “G” or “H” or “I” or “L” or “M” or “N” or “P” or “Q” or “R” or “V” or “W” or “Y”
  VARIANT (10)..(10): /replace=“D” or “E” or “F” or “G” or “H” or “I” or “K” or “L” or “N” or “P” or “Q” or “R” or “T” or “Y”
  VARIANT (11)..(11): /replace=“E” or “F” or “H” or “I” or “M” or “N” or “P” or “R” or “S” or “T” or “V” or “W” or “Y”
  VARIANT (12)..(12): /replace=“D” or “E” or “F” or “G” or “H” or “L” or “M” or “N” or “Q” or “T” or “V” or “W” or “Y”
  SITE (1)..(12): /note=“Variant residues given in the sequence have no preference with respect to those in the annotations for variant positions”
AAAAAAAAAACC

SEQ ID NO: 739 - protein, 9 aa, Artificial Sequence
  source: /note=“Description of Artificial Sequence: Synthetic peptide”
  VARIANT (1)..(1): /replace=“C” or “D” or “E” or “F” or “G” or “I” or “M” or “N” or “P” or “Q” or “S” or “T” or “V” or “W” or “Y”
  VARIANT (2)..(2): /replace=“C” or “D” or “E” or “F” or “G” or “I” or “M” or “N” or “P” or “Q” or “S” or “T” or “V” or “W” or “Y”
  VARIANT (3)..(3): /replace=“C” or “D” or “E” or “G” or “I” or “K” or “L” or “M” or “N” or “P” or “Q” or “R” or “T” or “V” or “W” or “Y”
  VARIANT (4)..(4): /replace=“E” or “F” or “P” or “Q” or “Y”
  VARIANT (5)..(5): /replace=“K” or “L” or “M” or “N” or “P” or “Q” or “R” or “S”
  VARIANT (6)..(6): /replace=“D” or “E” or “F” or “H” or “I” or “L” or “M” or “N” or “P” or “Q” or “S” or “T” or “V” or “Y”
  VARIANT (7)..(7): /replace=“C” or “D” or “E” or “F” or “G” or “I” or “K” or “L” or “M” or “N” or “P” or “Q” or “R” or “V” or “W” or “Y”
  VARIANT (8)..(8): /replace=“E” or “F” or “G” or “I” or “K” or “M” or “N” or “P” or “Q” or “R” or “T” or “V” or “W” or “Y”
  VARIANT (9)..(9): /replace=“D” or “E” or “F” or “G” or “H” or “I” or “K” or “L” or “M” or “N” or “Q” or “R” or “T” or “V” or “W” or “Y”
  SITE (1)..(9): /note=“Variant residues given in the sequence have no preference with respect to those in the annotations for variant positions”
AAADGCAAC

SEQ ID NO: 740 - protein, 10 aa, Artificial Sequence
  source: /note=“Description of Artificial Sequence: Synthetic peptide”
  VARIANT (1)..(1): /replace=“C” or “D” or “E” or “F” or “G” or “H” or “I” or “K” or “L” or “M” or “N” or “P” or “Q” or “R” or “S” or “T” or “V” or “W” or “Y”
  VARIANT (2)..(2): /replace=“C” or “D” or “E” or “F” or “G” or “H” or “K” or “L” or “M” or “N” or “P” or “Q” or “R” or “S” or “T” or “V” or “W”
  VARIANT (3)..(3): /replace=“C” or “D” or “E” or “F” or “G” or “H” or “I” or “K” or “L” or “M” or “N” or “P” or “Q” or “R” or “T” or “V” or “W” or “Y”
  VARIANT (4)..(4): /replace=“C” or “D” or “E” or “F” or “G” or “H” or “I” or “K” or “M” or “N” or “P” or “Q” or “R” or “S” or “T” or “V” or “W” or “Y”
  VARIANT (5)..(5): /replace=“K” or “L” or “M” or “P” or “Q” or “V” or “W” or “Y”
  VARIANT (6)..(6): /replace=“G” or “H” or “I” or “K” or “L” or “M” or “P” or “Q” or “T” or “V” or “W” or “Y”
  VARIANT (7)..(7): /replace=“C” or “D” or “E” or “F” or “G” or “H” or “I” or “K” or “M” or “N” or “P” or “Q” or “R” or “S” or “T” or “V” or “W” or “Y”
  VARIANT (8)..(8): /replace=“C” or “D” or “E” or “F” or “H” or “K” or “L” or “M” or “N” or “P” or “Q” or “R” or “T” or “V”
  VARIANT (9)..(9): /replace=“C” or “D” or “E” or “F” or “G” or “H” or “K” or “M” or “N” or “P” or “Q” or “R” or “S” or “T” or “V” or “W” or “Y”
  VARIANT (10)..(10): /replace=“C” or “D” or “E” or “F” or “I” or “K” or “L” or “M” or “P” or “Q” or “R” or “T” or “V” or “W” or “Y”
  SITE (1)..(10): /note=“Variant residues given in the sequence have no preference with respect to those in the annotations for variant positions”
AAAAIFAAAA

SEQ ID NO: 741 - protein, 17 aa, Artificial Sequence
  source: /note=“Description of Artificial Sequence: Synthetic peptide”
  VARIANT (1)..(1): /replace=“C” or “D” or “E” or “F” or “H” or “K” or “L” or “N” or “P” or “Q” or “R” or “S” or “Y”
  VARIANT (2)..(2): /replace=“C” or “D” or “E” or “G” or “H” or “I” or “K” or “L” or “M” or “N” or “P” or “Q” or “R” or “S” or “T” or “V” or “W” or “Y”
  VARIANT (3)..(3): /replace=“C” or “E” or “F” or “G” or “H” or “I” or “K” or “L” or “M” or “P” or “Q” or “R” or “S” or “V” or “Y”
  VARIANT (4)..(4): /replace=“D” or “E” or “F” or “G” or “H” or “I” or “K” or “L” or “M” or “N” or “Q” or “R” or “S” or “V”
  VARIANT (5)..(5): /replace=“C” or “F” or “H” or “K” or “L” or “M” or “P” or “Q” or “R” or “S” or “T” or “V” or “W”
  VARIANT (6)..(6): /replace=“C” or “E” or “F” or “G” or “H” or “I” or “K” or “L” or “M” or “P” or “Q” or “R” or “V” or “W”
  VARIANT (7)..(7): /replace=“C” or “D” or “E” or “F” or “G” or “H” or “I” or “K” or “L” or “M” or “N” or “P” or “Q” or “R” or “T” or “V” or “W” or “Y”
  VARIANT (8)..(8): /replace=“C” or “F” or “G” or “I” or “K” or “L” or “M” or “N” or “P” or “Q” or “R” or “S” or “T” or “V” or “W” or “Y”
  VARIANT (9)..(9): /replace=“D” or “E” or “F” or “G” or “H” or “K” or “L” or “M” or “N” or “P” or “Q” or “S” or “T” or “V” or “W” or “Y”
  VARIANT (10)..(10): /replace=“C” or “E” or “H” or “K” or “L” or “M” or “P” or “S” or “T” or “V” or “W” or “Y”
  VARIANT (11)..(11): /replace=“C” or “D” or “E” or “F” or “G” or “H” or “I” or “K” or “L” or “M” or “N” or “P” or “Q” or “R” or “T” or “V” or “W” or “Y”
  VARIANT (12)..(12): /replace=“C” or “D” or “E” or “F” or “H” or “I” or “K” or “L” or “M” or “N” or “P” or “Q” or “R” or “S” or “T” or “V” or “W” or “Y”
  VARIANT (13)..(13): /replace=“C” or “D” or “E” or “F” or “G” or “H” or “I” or “K” or “L” or “M” or “N” or “R” or “S” or “T” or “V” or “W” or “Y”
  VARIANT (14)..(14): /replace=“C” or “D” or “F” or “G” or “H” or “I” or “L” or “M” or “N” or “P” or “Q” or “R” or “S” or “T” or “V” or “W” or “Y”
  VARIANT (15)..(15): /replace=“C” or “D” or “E” or “G” or “H” or “I” or “K” or “L” or “M” or “N” or “P” or “Q” or “R” or “S” or “T” or “V” or “W” or “Y”
  VARIANT (16)..(16): /replace=“C” or “D” or “F” or “G” or “H” or “I” or “K” or “L” or “M” or “N” or “P” or “S” or “T” or “V” or “W” or “Y”
  VARIANT (17)..(17): /replace=“C” or “E” or “F” or “G” or “H” or “I” or “K” or “L” or “M” or “N” or “P” or “Q” or “R” or “S” or “T” or “V” or “W” or “Y”
  SITE (1)..(17): /note=“Variant residues given in the sequence have no preference with respect to those in the annotations for variant positions”
AAACAAAACAAAAAAAA

SEQ ID NO: 742 - protein, 15 aa, Artificial Sequence
  source: /note=“Description of Artificial Sequence: Synthetic peptide”
  VARIANT (1)..(1): /replace=“C” or “D” or “E” or “F” or “G” or “H” or “I” or “K” or “L” or “M” or “N” or “P” or “Q” or “R” or “S” or “T” or “V” or “W” or “Y”
  VARIANT (2)..(2): /replace=“G” or “H” or “I” or “T”
  VARIANT (3)..(3): /replace=“C” or “D” or “F” or “G” or “H” or “I” or “K” or “L” or “M” or “N” or “P” or “Q” or “R” or “S” or “T” or “V” or “W” or “Y”
  VARIANT (4)..(4): /replace=“E” or “F” or “H” or “N” or “Q” or “R” or “S” or “T” or “V” or “W” or “Y”
  VARIANT (5)..(5): /replace=“H” or “I” or “L” or “M” or “N” or “Q” or “Y”
  VARIANT (6)..(6): /replace=“C” or “D” or “F” or “G” or “H” or “K” or “M” or “N” or “P” or “Q” or “R” or “S” or “T” or “V” or “Y”
  VARIANT (7)..(7): /replace=“C” or “E” or “F” or “H” or “K” or “M” or “N” or “P” or “Q” or “S” or “T” or “W” or “Y”
  VARIANT (8)..(8): /replace=“C” or “D” or “E” or “F” or “G” or “H” or “I” or “K” or “L” or “M” or “N” or “P” or “Q” or “S” or “T” or “V” or “W”
  VARIANT (9)..(9): /replace=“C” or “D” or “E” or “F” or “H” or “I” or “K” or “L” or “N” or “Q” or “R” or “S” or “V” or “W” or “Y”
  VARIANT (10)..(10): /replace=“C” or “D” or “E” or “F” or “H” or “I” or “K” or “L” or “M” or “N” or “P” or “Q” or “R” or “S” or “V” or “W” or “Y”
  VARIANT (11)..(11): /replace=“C” or “E” or “F” or “H” or “I” or “K” or “L” or “M” or “N” or “P” or “Q” or “S” or “T” or “V” or “W” or “Y”
  VARIANT (12)..(12): /replace=“H” or “I” or “K” or “N” or “P” or “Q” or “R” or “V” or “W” or “Y”
  VARIANT (13)..(13): /replace=“C” or “D” or “E” or “F” or “G” or “H” or “I” or “K” or “L” or “N” or “P” or “Q” or “R” or “S” or “T” or “V” or “W” or “Y”
  VARIANT (14)..(14): /replace=“F” or “G” or “H” or “P” or “Q” or “T”
  VARIANT (15)..(15): /replace=“E” or “F” or “G” or “I” or “K” or “L” or “N” or “P” or “Q” or “R” or “S” or “T”
  SITE (1)..(15): /note=“Variant residues given in the sequence have no preference with respect to those in the annotations for variant positions”
AFADAAAAAAAFADD

SEQ ID NO: 743 - protein, 11 aa, Artificial Sequence
  source: /note=“Description of Artificial Sequence: Synthetic peptide”
  VARIANT (1)..(1): /replace=“C” or “D” or “E” or “F” or “G” or “H” or “I” or “L” or “M” or “N” or “P” or “R” or “S” or “T” or “V” or “W” or “Y”
  VARIANT (2)..(2): /replace=“C” or “D” or “E” or “F” or “G” or “H” or “I” or “K” or “L” or “M” or “N” or “Q” or “R” or “S” or “V” or “W” or “Y”
  VARIANT (3)..(3): /replace=“C” or “D” or “E” or “F” or “G” or “H” or “I” or “K” or “L” or “M” or “N” or “Q” or “S” or “T” or “V” or “W” or “Y”
  VARIANT (4)..(4): /replace=“C” or “D” or “E” or “F” or “G” or “I” or “K” or “M” or “N” or “R” or “S” or “T” or “V” or “W” or “Y”
  VARIANT (5)..(5): /replace=“D” or “E” or “H” or “K” or “L” or “M” or “Q” or “T” or “W” or “Y”
  VARIANT (6)..(6): /replace=“C” or “D” or “E” or “F” or “G” or “H” or “K” or “M” or “N” or “P” or “Q” or “R” or “T” or “V” or “W” or “Y”
  VARIANT (7)..(7): /replace=“E” or “G” or “I” or “L” or “M” or “P” or “Q” or “V” or “W” or “Y”
  VARIANT (8)..(8): /replace=“M” or “P” or “Q” or “T” or “W”
  VARIANT (9)..(9): /replace=“C” or “E” or “F” or “G” or “I” or “K” or “L” or “M” or “N” or “P” or “Q” or “R” or “T” or “V” or “Y”
  VARIANT (10)..(10): /replace=“C” or “D” or “E” or “F” or “G” or “H” or “K” or “L” or “M” or “N” or “P” or “Q” or “R” or “S” or “T” or “W” or “Y”
  VARIANT (11)..(11): /replace=“D” or “E” or “F” or “G” or “I” or “L” or “M” or “N” or “P” or “Q” or “R” or “W” or “Y”
  SITE (1)..(11): /note=“Variant residues given in the sequence have no preference with respect to those in the annotations for variant positions”
AAAACACCAAC

SEQ ID NO: 744 - protein, 12 aa, Artificial Sequence
  source: /note=“Description of Artificial Sequence: Synthetic peptide”
  VARIANT (1)..(1): /replace=“C” or “D” or “E” or “F” or “G” or “H” or “K” or “L” or “N” or “P” or “R” or “S” or “W” or “Y”
  VARIANT (2)..(2): /replace=“C” or “D” or “E” or “G” or “H” or “K” or “M” or “N” or “Q” or “T” or “W”
  VARIANT (3)..(3): /replace=“C” or “D” or “E” or “F” or “G” or “H” or “K” or “L” or “M” or “N” or “P” or “Q” or “R” or “S” or “T” or “W” or “Y”
  VARIANT (4)..(4): /replace=“C” or “D” or “E” or “F” or “G” or “H” or “I” or “K” or “M” or “N” or “P” or “Q” or “R” or “S” or “V” or “W” or “Y”
  VARIANT (6)..(6): /replace=“H” or “I” or “L” or “M” or “N” or “P” or “Q” or “W” or “Y”
  VARIANT (7)..(7): /replace=“D” or “E” or “F” or “G” or “H” or “I” or “K” or “L” or “M” or “N” or “P” or “Q” or “R” or “S” or “T” or “V” or “W” or “Y”
  VARIANT (8)..(8): /replace=“D” or “E” or “G” or “H” or “I” or “K” or “L” or “M” or “P” or “Q” or “R” or “S” or “W” or “Y”
  VARIANT (10)..(10): /replace=“C” or “D” or “E” or “G” or “H” or “I” or “K” or “L” or “M” or “N” or “P” or “Q” or “T” or “W” or “Y”
  VARIANT (11)..(11): /replace=“E” or “G” or “I” or “L” or “M” or “P” or “R” or “S” or “T” or “W” or “Y”
  VARIANT (12)..(12): /replace=“E” or “F” or “G” or “H” or “I” or “K” or “L” or “M” or “P” or “Q” or “V” or “W” or “Y”
  SITE (1)..(12): /note=“Variant residues given in the sequence have no preference with respect to those in the annotations for variant positions”
AAAALCCCIACA

SEQ ID NO: 745 - protein, 9 aa, Artificial Sequence
  source: /note=“Description of Artificial Sequence: Synthetic peptide”
  VARIANT (1)..(1): /replace=“D” or “F” or “G” or “I” or “K” or “M” or “N” or “P” or “Q” or “S” or “T” or “V” or “W” or “Y”
  VARIANT (2)..(2): /replace=“C” or “D” or “F” or “G” or “I” or “L” or “M” or “N” or “P” or “Q” or “R” or “S” or “T” or “V” or “W”
  VARIANT (3)..(3): /replace=“E” or “G” or “K” or “M” or “P” or “S” or “V” or “W”
  VARIANT (4)..(4): /replace=“H” or “L” or “M” or “P” or “Q” or “R” or “V” or “W”
  VARIANT (5)..(5): /replace=“D” or “E” or “F” or “L” or “M” or “P” or “V” or “W”
  VARIANT (6)..(6): /replace=“C” or “E” or “G” or “H” or “K” or “M” or “N” or “P” or “Q” or “R” or “V” or “W”
  VARIANT (7)..(7): /replace=“C” or “D” or “E” or “G” or “H” or “I” or “K” or “M” or “N” or “P” or “R” or “S” or “T” or “V” or “W” or “Y”
  VARIANT (8)..(8): /replace=“C” or “D” or “E” or “G” or “K” or “M” or “N” or “P” or “Q” or “R” or “S” or “W”
  VARIANT (9)..(9): /replace=“D” or “E” or “F” or “G” or “H” or “K” or “L” or “M” or “Q” or “R” or “T” or “V” or “W” or “Y”
  SITE (1)..(9): /note=“Variant residues given in the sequence have no preference with respect to those in the annotations for variant positions”
CACCCAAAC

SEQ ID NO: 746-753 - skipped (8 entries)

SEQ ID NO: 754 - protein, 8 aa, Artificial Sequence
  source: /note=“Description of Artificial Sequence: Synthetic peptide”
  VARIANT (1)..(1): /replace=“E” or “F” or “G” or “H” or “I” or “K” or “L” or “M” or “N” or “P” or “Q” or “R” or “T” or “V” or “W” or “Y”
  VARIANT (2)..(2): /replace=“C” or “D” or “E” or “F” or “G” or “H” or “K” or “M” or “N” or “P” or “Q” or “R” or “T” or “W”
  VARIANT (3)..(3): /replace=“C” or “D” or “E” or “F” or “G” or “H” or “K” or “L” or “M” or “P” or “Q” or “R” or “T” or “V” or “W” or “Y”
  VARIANT (4)..(4): /replace=“C” or “D” or “E” or “F” or “G” or “H” or “K” or “M” or “N” or “P” or “Q” or “R” or “T” or “W”
  VARIANT (5)..(5): /replace=“C” or “D” or “E” or “G” or “H” or “I” or “K” or “L” or “M” or “N” or “Q” or “R” or “S” or “V” or “W”
  VARIANT (6)..(6): /replace=“D” or “E” or “F” or “G” or “H” or “I” or “L” or “N” or “P” or “Q” or “T” or “V” or “W” or “Y”
  VARIANT (7)..(7): /replace=“D” or “E” or “F” or “G” or “I” or “K” or “L” or “M” or “P” or “Q” or “R” or “S” or “T” or “V” or “W” or “Y”
  VARIANT (8)..(8): /replace=“F” or “G” or “H” or “I” or “K” or “L” or “M” or “N” or “P” or “Q” or “R” or “T” or “W” or “Y”
  SITE (1)..(8): /note=“Variant residues given in the sequence have no preference with respect to those in the annotations for variant positions”
AAAAACAE

SEQ ID NO: 755 - protein, 8 aa, Artificial Sequence
  source: /note=“Description of Artificial Sequence: Synthetic peptide”
  VARIANT (1)..(1): /replace=“C” or “D” or “E” or “G” or “H” or “I” or “K” or “L” or “M” or “P” or “Q” or “R” or “W” or “Y”
  VARIANT (3)..(3): /replace=“D”
  VARIANT (4)..(4): /replace=“E” or “A”
  VARIANT (5)..(5): /replace=“E” or “F” or “G” or “H” or “I” or “K” or “L” or “M” or “N” or “P” or “Q” or “T” or “W” or “Y”
  VARIANT (6)..(6): /replace=“R”
  VARIANT (7)..(7): /replace=“D” or “E” or “F” or “G” or “I” or “K” or “L” or “M” or “P” or “Q” or “R” or “T” or “V” or “W” or “Y”
  VARIANT (8)..(8): /replace=“C” or “D” or “F” or “G” or “H” or “I” or “K” or “L” or “P” or “Q” or “S” or “V” or “W” or “Y”
  SITE (1)..(8): /note=“Variant residues given in the sequence have no preference with respect to those in the annotations for variant positions”
  source: /note=“See specification as filed for detailed description of substitutions and preferred embodiments”
ARCGDKAA

SEQ ID NO: 756 - protein, 13 aa, Artificial Sequence
  source: /note=“Description of Artificial Sequence: Synthetic peptide”
  VARIANT (1)..(1): /replace=“C” or “D” or “F” or “G” or “H” or “I” or “K” or “L” or “M” or “N” or “Q” or “R” or “W” or “Y”
  VARIANT (2)..(2): /replace=“C” or “D” or “E” or “F” or “H” or “L” or “M” or “N” or “P” or “Q” or “R” or “V” or “W” or “Y”
  VARIANT (3)..(3): /replace=“D” or “F” or “I” or “Q”
  VARIANT (4)..(4): /replace=“F” or “G” or “H” or “I” or “K” or “L” or “M” or “N” or “P” or “Q”
  VARIANT (5)..(5): /replace=“D” or “E” or “F” or “H” or “I” or “K” or “L” or “M” or “P” or “Q” or “S” or “T” or “V” or “W” or “Y”
  VARIANT (6)..(6): /replace=“E” or “F” or “G” or “H” or “I” or “K” or “L” or “M” or “N” or “P” or “Q” or “R” or “T” or “W” or “Y”
  VARIANT (8)..(8): /replace=“C” or “D” or “F” or “G” or “H” or “I” or “K” or “L” or “M” or “N” or “P” or “Q” or “S” or “T” or “W” or “Y”
  VARIANT (9)..(9): /replace=“E” or “G” or “I” or “K” or “L” or “M” or “P” or “Q” or “R” or “T” or “V” or “W” or “Y”
  VARIANT (10)..(10): /replace=“C” or “E” or “F” or “H” or “I” or “K” or “L” or “M” or “N” or “Q” or “R”
  VARIANT (11)..(11): /replace=“F” or “G” or “H” or “M” or “N” or “P” or “R” or “T” or “W”
  VARIANT (12)..(12): /replace=“K”
  VARIANT (13)..(13): /replace=“H” or “I” or “K” or “M” or “P” or “Q” or “R” or “W” or “Y”
  SITE (1)..(13): /note=“Variant residues given in the sequence have no preference with respect to those in the annotations for variant positions”
  source: /note=“See specification as filed for detailed description of substitutions and preferred embodiments”
AACEAAYAAADRG

SEQ ID NO: 757 - protein, 6 aa, Artificial Sequence
  source: /note=“Description of Artificial Sequence: Synthetic peptide”
  VARIANT (1)..(1): /replace=“C” or “D” or “E” or “F” or “G” or “I” or “K” or “L” or “M” or “N” or “Q” or “S” or “T” or “V” or “W” or “Y”
  VARIANT (2)..(2): /replace=“C” or “D” or “E” or “F” or “H” or “I” or “K” or “L” or “M” or “N” or “P” or “Q” or “R” or “T” or “V” or “W”
  VARIANT (3)..(3): /replace=“C” or “D” or “E” or “F” or “G” or “H” or “I” or “K” or “M” or “P” or “Q” or “R” or “V” or “W” or “Y”
  VARIANT (4)..(4): /replace=“G” or “K” or “M” or “T” or “V” or “W”
  VARIANT (5)..(5): /replace=“F” or “G” or “L” or “M” or “P” or “Q” or “S” or “T” or “V” or “W” or “Y”
  VARIANT (6)..(6): /replace=“C” or “G” or “Q” or “S” or “T” or “W”
  SITE (1)..(6): /note=“Variant residues given in the sequence have no preference with respect to those in the annotations for variant positions”
  source: /note=“See specification as filed for detailed description of substitutions and preferred embodiments”
AAAEDA

SEQ ID NO: 758 - protein, 3 aa, Artificial Sequence
  source: /note=“Description of Artificial Sequence: Synthetic peptide”
  VARIANT (1)..(1): /replace=“G” or “H” or “N” or “R” or “S”
  VARIANT (2)..(2): /replace=“S” or “T”
  VARIANT (3)..(3): /replace=“D” or “E” or “F” or “H” or “N” or “P” or “T” or “V” or “W”
  SITE (1)..(3): /note=“Variant residues given in the sequence have no preference with respect to those in the annotations for variant positions”
  source: /note=“See specification as filed for detailed description of substitutions and preferred embodiments”
ADA

SEQ ID NO: 759 - protein, 9 aa, Artificial Sequence
  source: /note=“Description of Artificial Sequence: Synthetic peptide”
  VARIANT (1)..(1): /replace=“C” or “D” or “E” or “F” or “G” or “I” or “M” or “N” or “P” or “Q” or “S” or “T” or “V” or “W” or “Y”
  VARIANT (2)..(2): /replace=“C” or “D” or “E” or “F” or “G” or “I” or “M” or “N” or “P” or “Q” or “S” or “T” or “V” or “W” or “Y”
  VARIANT (3)..(3): /replace=“C” or “D” or “E” or “G” or “I” or “K” or “L” or “M” or “N” or “P” or “Q” or “R” or “T” or “V” or “W” or “Y”
  VARIANT (4)..(4): /replace=“Q” or “Y”
  VARIANT (5)..(5): /replace=“R” or “K”
  VARIANT (6)..(6): /replace=“E” or “L” or “M” or “N” or “Q” or “S” or “T” or “V”
  VARIANT (7)..(7): /replace=“C” or “D” or “E” or “F” or “G” or “I” or “K” or “L” or “M” or “N” or “P” or “Q” or “R” or “V” or “W” or “Y”
  VARIANT (8)..(8): /replace=“E” or “F” or “G” or “I” or “K” or “M” or “N” or “P” or “Q” or “R” or “T” or “V” or “W” or “Y”
  VARIANT (9)..(9): /replace=“D” or “E” or “F” or “G” or “H” or “I” or “K” or “L” or “M” or “N” or “Q” or “R” or “T” or “V” or “W” or “Y”
  SITE (1)..(9): /note=“Variant residues given in the sequence have no preference with respect to those in the annotations for variant positions”
  source: /note=“See specification as filed for detailed description of substitutions and preferred embodiments”
AAAEGDAAC

SEQ ID NO: 760 - protein, 8 aa, Artificial Sequence
  source: /note=“Description of Artificial Sequence: Synthetic peptide”
  VARIANT (3)..(3): /replace=“C” or “D” or “E” or “F” or “G” or “H” or “I” or “K” or “L” or “M” or “N” or “P” or “Q” or “R” or “T” or “V” or “W” or “Y”
  VARIANT (4)..(4): /replace=“C” or “D” or “E” or “F” or “G” or “H” or “I” or “K” or “M” or “N” or “P” or “Q” or “R” or “S” or “T” or “V” or “W” or “Y”
  VARIANT (6)..(6): /replace=“G” or “H” or “I” or “K” or “L” or “M” or “P” or “Q” or “T” or “V” or “W” or “Y”
  VARIANT (7)..(7): /replace=“N” or “S”
  VARIANT (8)..(8): /replace=“C” or “D” or “E” or “F” or “H” or “K” or “L” or “M” or “N” or “P” or “Q” or “R” or “T” or “V”
  SITE (1)..(8): /note=“Variant residues given in the sequence have no preference with respect to those in the annotations for variant positions”
RGAAKFFA

SEQ ID NO: 761 - protein, 8 aa, Artificial Sequence
  source: /note=“Description of Artificial Sequence: Synthetic peptide”
  VARIANT (1)..(1): /replace=“C” or “D” or “E” or “G” or “H” or “I” or “K” or “L” or “M” or “N” or “P” or “Q” or “R” or “S” or “T” or “V” or “W” or “Y”
  VARIANT (2)..(2): /replace=“C” or “E” or “F” or “G” or “H” or “I” or “K” or “L” or “M” or “P” or “Q” or “R” or “S” or “V” or “Y”
  VARIANT (3)..(3): /replace=“D” or “E” or “F” or “G” or “H” or “I” or “K” or “L” or “M” or “N” or “Q” or “R” or “S” or “V”
  VARIANT (6)..(6): /replace=“C” or “D” or “E” or “F” or “G” or “H” or “I” or “K” or “L” or “M” or “N” or “P” or “Q” or “R” or “T” or “V” or “W” or “Y”
  VARIANT (7)..(7): /replace=“K” or “N” or “R” or “S” or “T”
  VARIANT (8)..(8): /replace=“D” or “E” or “F” or “G” or “H” or “K” or “L” or “M” or “N” or “P” or “Q” or “S” or “T” or “V” or “W” or “Y”
  SITE (1)..(8): /note=“Variant residues given in the sequence have no preference with respect to those in the annotations for variant positions”
  source: /note=“See specification as filed for detailed description of substitutions and preferred embodiments”
AACMFAGC

SEQ ID NO: 762 - protein, 15 aa, Artificial Sequence
  source: /note=“Description of Artificial Sequence: Synthetic peptide”
  VARIANT (1)..(1): /replace=“C” or “D” or “E” or “F” or “G” or “H” or “I” or “K” or “L” or “M” or “N” or “P” or “Q” or “R” or “S” or “T” or “V” or “W” or “Y”
  VARIANT (2)..(2): /replace=“G” or “H” or “I” or “T”
  VARIANT (3)..(3): /replace=“C” or “D” or “F” or “G” or “H” or “I” or “K” or “L” or “M” or “N” or “P” or “Q” or “R” or “S” or “T” or “V” or “W” or “Y”
  VARIANT (5)..(5): /replace=“H” or “I” or “L” or “M” or “N” or “Q” or “Y”
  VARIANT (6)..(6): /replace=“C” or “D” or “F” or “G” or “H” or “K” or “M” or “N” or “P” or “Q” or “R” or “S” or “T” or “V” or “Y”
  VARIANT (7)..(7): /replace=“C” or “E” or “F” or “H” or “K” or “M” or “N” or “P” or “Q” or “S” or “T” or “W” or “Y”
  VARIANT (8)..(8): /replace=“C” or “D” or “E” or “F” or “G” or “H” or “I” or “K” or “L” or “M” or “N” or “P” or “Q” or “S” or “T” or “V” or “W”
  VARIANT (9)..(9): /replace=“C” or “D” or “E” or “F” or “H” or “I” or “K” or “L” or “N” or “Q” or “R” or “S” or “V” or “W” or “Y”
  VARIANT (10)..(10): /replace=“C” or “D” or “E” or “F” or “H” or “I” or “K” or “L” or “M” or “N” or “P” or “Q” or “R” or “S” or “V” or “W” or “Y”
  VARIANT (11)..(11): /replace=“C” or “E” or “F” or “H” or “I” or “K” or “L” or “M” or “N” or “P” or “Q” or “S” or “T” or “V” or “W” or “Y”
  VARIANT (12)..(12): /replace=“Y”
  VARIANT (13)..(13): /replace=“C” or “D” or “E” or “F” or “G” or “H” or “I” or “K” or “L” or “N” or “P” or “Q” or “R” or “S” or “T” or “V” or “W” or “Y”
  VARIANT (15)..(15): /replace=“E” or “F” or “G” or “I” or “K” or “L” or “N” or “P” or “Q” or “R” or “S” or “T”
  SITE (1)..(15): /note=“Variant residues given in the sequence have no preference with respect to those in the annotations for variant positions”
AFADAAAAAAAFATD

SEQ ID NO: 763 - protein, 6 aa, Artificial Sequence
  source: /note=“Description of Artificial Sequence: Synthetic peptide”
  VARIANT (3)..(3): /replace=“C” or “D” or “E” or “F” or “G” or “H” or “K” or “M” or “N” or “P” or “Q” or “R” or “T” or “V” or “W” or “Y”
  VARIANT (4)..(4): /replace=“E” or “G” or “I” or “L” or “M” or “P” or “Q” or “V” or “W” or “Y”
  SITE (1)..(6): /note=“Variant residues given in the sequence have no preference with respect to those in the annotations for variant positions”
EDACPY

SEQ ID NO: 764 - protein, 3 aa, Artificial Sequence
  source: /note=“Description of Artificial Sequence: Synthetic peptide”
  VARIANT (1)..(1): /replace=“Q”
  VARIANT (2)..(2): /replace=“T” or “V”
  VARIANT (3)..(3): /replace=“D” or “E” or “G” or “H” or “I” or “K” or “L” or “M” or “P” or “Q” or “R” or “S” or “W” or “Y”
  SITE (1)..(3): /note=“Variant residues given in the sequence have no preference with respect to those in the annotations for variant positions”
  source: /note=“See specification as filed for detailed description of substitutions and preferred embodiments”
HSC

SEQ ID NO: 765 - protein, 9 aa, Artificial Sequence
  source: /note=“Description of Artificial Sequence: Synthetic peptide”
  VARIANT (1)..(1): /replace=“D” or “F” or “G” or “I” or “K” or “M” or “N” or “P” or “Q” or “S” or “T” or “V” or “W” or “Y”
  VARIANT (2)..(2): /replace=“C” or “D” or “F” or “G” or “I” or “L” or “M” or “N” or “P” or “Q” or “R” or “S” or “T” or “V” or “W”
  VARIANT (4)..(4): /replace=“H” or “L” or “M” or “P” or “Q” or “R” or “V” or “W”
  VARIANT (6)..(6): /replace=“N” or “P” or “R” or “W”
  VARIANT (7)..(7): /replace=“C” or “D” or “E” or “G” or “H” or “I” or “K” or “M” or “N” or “P” or “R” or “S” or “T” or “V” or “W” or “Y”
  VARIANT (8)..(8): /replace=“P” or “S” or “W”
  VARIANT (9)..(9): /replace=“D” or “E” or “F” or “G” or “H” or “K” or “L” or “M” or “Q” or “R” or “T” or “V” or “W” or “Y”
  SITE (1)..(9): /note=“Variant residues given in the sequence have no preference with respect to those in the annotations for variant positions”
CAPCFAADC

SEQ ID NO: 766-773 - skipped (8 entries)

SEQ ID NO: 774 - protein, 10 aa, Artificial Sequence
  source: /note=“Description of Artificial Sequence: Synthetic peptide”
  VARIANT (1)..(1): /replace=“E” or “F” or “G” or “H” or “I” or “K” or “L” or “M” or “N” or “P” or “Q” or “R” or “T” or “V” or “W” or “Y”
  VARIANT (2)..(2): /replace=“C” or “D” or “E” or “F” or “G” or “H” or “K” or “M” or “N” or “P” or “Q” or “R” or “T” or “W”
  VARIANT (3)..(3): /replace=“C” or “D” or “E” or “F” or “G” or “H” or “K” or “L” or “M” or “P” or “Q” or “R” or “T” or “V” or “W” or “Y”
  VARIANT (4)..(4): /replace=“C” or “D” or “E” or “F” or “G” or “H” or “K” or “M” or “N” or “P” or “Q” or “R” or “T” or “W”
  VARIANT (5)..(5): /replace=“C” or “D” or “E” or “G” or “H” or “I” or “K” or “L” or “M” or “N” or “Q” or “R” or “S” or “V” or “W”
  VARIANT (6)..(6): /replace=“D” or “E” or “F” or “G” or “H” or “I” or “L” or “N” or “P” or “Q” or “T” or “V” or “W” or “Y”
  VARIANT (7)..(7): /replace=“D” or “E” or “F” or “G” or “I” or “K” or “L” or “M” or “P” or “Q” or “R” or “S” or “T” or “V” or “W” or “Y”
  VARIANT (8)..(8): /replace=“F” or “G” or “H” or “I” or “K” or “L” or “M” or “N” or “P” or “Q” or “R” or “T” or “W” or “Y”
  VARIANT (9)..(9): /replace=“C” or “D” or “E” or “F” or “G” or “H” or “M” or “N” or “P” or “Q” or “R” or “S” or “W” or “Y”
  VARIANT (10)..(10): /replace=“C” or “D” or “E” or “F” or “G” or “H” or “I” or “K” or “M” or “S” or “T” or “V” or “W”
  SITE (1)..(10): /note=“Variant residues given in the sequence have no preference with respect to those in the annotations for variant positions”
AAAAACAEAA

SEQ ID NO: 775 - protein, 18 aa, Artificial Sequence
  source: /note=“Description of Artificial Sequence: Synthetic peptide”
  VARIANT (1)..(1): /replace=“C” or “E” or “F” or “G” or “H” or “I” or “K” or “L” or “M” or “N” or “Q” or “R” or “W” or “Y”
  VARIANT (2)..(2): /replace=“D” or “F” or “H” or “I” or “K” or “N” or “P” or “Q” or “R” or “S” or “T” or “V” or “W” or “Y”
  VARIANT (3)..(3): /replace=“C” or “D” or “E” or “G” or “H” or “I” or “K” or “L” or “M” or “P” or “Q” or “R” or “W” or “Y”
  VARIANT (5)..(5): /replace=“D”
  VARIANT (6)..(6): /replace=“E” or “A”
  VARIANT (7)..(7): /replace=“E” or “F” or “G” or “H” or “I” or “K” or “L” or “M” or “N” or “P” or “Q” or “T” or “W” or “Y”
  VARIANT (8)..(8): /replace=“R”
  VARIANT (9)..(9): /replace=“D” or “E” or “F” or “G” or “I” or “K” or “L” or “M” or “P” or “Q” or “R” or “T” or “V” or “W” or “Y”
  VARIANT (10)..(10): /replace=“C” or “D” or “F” or “G” or “H” or “I” or “K” or “L” or “P” or “Q” or “S” or “V” or “W” or “Y”
  VARIANT (11)..(11): /replace=“G” or “I” or “L” or “M” or “P” or “R” or “V” or “W” or “Y”
  VARIANT (12)..(12): /replace=“E” or “G” or “I” or “K” or “L” or “M” or “P” or “Q” or “R” or “T” or “V” or “W” or “Y”
  VARIANT (14)..(14): /replace=“D” or “F” or “I” or “K” or “L” or “M” or “P” or “Q” or “R” or “S” or “T” or “W”
  VARIANT (15)..(15): /replace=“D” or “E” or “F” or “H” or “K” or “L” or “M” or “P” or “Q” or “S” or “V” or “W” or “Y”
  VARIANT (17)..(17): /replace=“C” or “D” or “F” or “G” or “H” or “K” or “L” or “M” or “S” or “V” or “W” or “Y”
  VARIANT (18)..(18): /replace=“F” or “G” or “H” or “I” or “K” or “L” or “M” or “N” or “P” or “Q” or “S” or “T” or “W” or “Y”
  SITE (1)..(18): /note=“Variant residues given in the sequence have no preference with respect to those in the annotations for variant positions”
  source: /note=“See specification as filed for detailed description of substitutions and preferred embodiments”
ACARCGDKAAAAVCAFAE

SEQ ID NO: 776 - protein, 13 aa, Artificial Sequence
  source: /note=“Description of Artificial Sequence: Synthetic peptide”
  VARIANT (1)..(1): /replace=“C” or “D” or “F” or “G” or “H” or “I” or “K” or “L” or “M” or “N” or “Q” or “R” or “W” or “Y”
  VARIANT (2)..(2): /replace=“C” or “D” or “E” or “F” or “H” or “L” or “M” or “N” or “P” or “Q” or “R” or “V” or “W” or “Y”
  VARIANT (3)..(3): /replace=“D” or “F” or “I” or “Q”
  VARIANT (4)..(4): /replace=“F” or “G” or “H” or “I” or “K” or “L” or “M” or “N” or “P” or “Q”
  VARIANT (5)..(5): /replace=“D” or “E” or “F” or “H” or “I” or “K” or “L” or “M” or “P” or “Q” or “S” or “T” or “V” or “W” or “Y”
  VARIANT (6)..(6): /replace=“E” or “F” or “G” or “H” or “I” or “K” or “L” or “M” or “N” or “P” or “Q” or “R” or “T” or “W” or “Y”
  VARIANT (8)..(8): /replace=“C” or “D” or “F” or “G” or “H” or “I” or “K” or “L” or “M” or “N” or “P” or “Q” or “S” or “T” or “W” or “Y”
  VARIANT (9)..(9): /replace=“E” or “G” or “I” or “K” or “L” or “M” or “P” or “Q” or “R” or “T” or “V” or “W” or “Y”
  VARIANT (10)..(10): /replace=“C” or “E” or “F” or “H” or “I” or “K” or “L” or “M” or “N” or “Q” or “R”
  VARIANT (11)..(11): /replace=“F” or “G” or “H” or “M” or “N” or “P” or “R” or “T” or “W”
  VARIANT (12)..(12): /replace=“K”
  VARIANT (13)..(13): /replace=“H” or “I” or “K” or “M” or “P” or “Q” or “R” or “W” or “Y”
  SITE (1)..(13): /note=“Variant residues given in the sequence have no preference with respect to those in the annotations for variant positions”
AACEAAYAAADRG

SEQ ID NO: 777 - protein, 11 aa, Artificial Sequence
  source: /note=“Description of Artificial Sequence: Synthetic peptide”
  VARIANT (1)..(1): /replace=“C” or “D” or “E” or “F” or “G” or “I” or “L” or “M” or “N” or “P” or “Q” or “S” or “T” or “V” or “W” or “Y”
  VARIANT (2)..(2): /replace=“C” or “D” or “F” or “G” or “H” or “I” or “K” or “L” or “M” or “N” or “P” or “Q” or “R” or “T” or “V” or “W” or “Y”
  VARIANT (3)..(3): /replace=“D” or “E” or “F” or “H” or “K” or “L” or “M” or “N” or “P” or “Q” or “S” or “T” or “V” or “W” or “Y”
  VARIANT (4)..(4): /replace=“C” or “D” or “E” or “F” or “G” or “I” or “K” or “L” or “M” or “N” or “Q” or “S” or “T” or “V” or “W” or “Y”
  VARIANT (5)..(5): /replace=“C” or “D” or “E” or “F” or “H” or “I” or “K” or “L” or “M” or “N” or “P” or “Q” or “R” or “T” or “V” or “W”
  VARIANT (6)..(6): /replace=“C” or “D” or “E” or “F” or “G” or “H” or “I” or “K” or “M” or “P” or “Q” or “R” or “V” or “W” or “Y”
  VARIANT (7)..(7): /replace=“G” or “K” or “M” or “T” or “V” or “W”
  VARIANT (8)..(8): /replace=“F” or “G” or “L” or “M” or “P” or “Q” or “S” or “T” or “V” or “W” or “Y”
  VARIANT (9)..(9): /replace=“C” or “G” or “Q” or “S” or “T” or “W”
  VARIANT (10)..(10): /replace=“C” or “D” or “E” or “F” or “G” or “H” or “K” or “L” or “M” or “N” or “P” or “Q” or “R” or “S” or “T” or “W” or “Y”
  VARIANT (11)..(11): /replace=“C” or “D” or “E” or “F” or “G” or “H” or “L” or “M” or “N” or “P” or “Q” or “R” or “S” or “T” or “V” or “W” or “Y”
  SITE (1)..(11): /note=“Variant residues given in the sequence have no preference with respect to those in the annotations for variant positions”
  source: /note=“See specification as filed for detailed description of substitutions and preferred embodiments”
AAAAAAEDAAA

SEQ ID NO: 778 - protein, 12 aa, Artificial Sequence
  source: /note=“Description of Artificial Sequence: Synthetic peptide”
  VARIANT (1)..(1): /replace=“R”
  VARIANT (2)..(2): /replace=“C” or “D” or “E” or “F” or “G” or “I” or “K” or “M” or “N” or “P” or “Q” or “R” or “S” or “V” or “W” or “Y”
  VARIANT (3)..(3): /replace=“C” or “D” or “E” or “F” or “G” or “H” or “I” or “K” or “L” or “N” or “S” or “T” or “V” or “W” or “Y”
  VARIANT (4)..(4): /replace=“C” or “E” or “H” or “K” or “L” or “M” or “N” or “P” or“Q” or “R” or “S” or “T” or “V” or “W” or “Y”
  VARIANT (5)..(5): /replace=“D” or “E” or “F” or “G” or “H” or “K” or “N” or “P” or “Q” or “R” or “T” or “W”
  VARIANT (6)..(6): /replace=“G” or “H” or “N” or “R” or “S”
  VARIANT (7)..(7): /replace=“S” or “T”
  VARIANT (8)..(8): /replace=“D” or “E” or “F” or “H” or “N” or “P” or “T” or “V” or “W”
  VARIANT (9)..(9): /replace=“Y”
  VARIANT (11)..(11): /replace=“E” or “F” or “H” or “I” or “M” or “N” or “P” or “R” or “S” or “T” or “V” or “W” or “Y”
  VARIANT (12)..(12): /replace=“D” or “E” or “F” or “G” or “H” or “L” or “M” or “N” or “Q” or “T” or “V” or “W” or “Y”
  SITE (1)..(12): /note=“Variant residues given in the sequence have no preference with respect to those in the annotations for variant positions”
  source: /note=“See specification as filed for detailed description of substitutions and preferred embodiments”
KAAAAADAIFCC

SEQ ID NO: 779 - protein, 9 aa, Artificial Sequence
  source: /note=“Description of Artificial Sequence: Synthetic peptide”
  VARIANT (1)..(1): /replace=“C” or “D” or “E” or “F” or “G” or “I” or “M” or “N” or “P” or “Q” or “S” or “T” or “V” or “W” or “Y”
  VARIANT (2)..(2): /replace=“C” or “D” or “E” or “F” or “G” or “I” or “M” or “N” or “P” or “Q” or “S” or “T” or “V” or “W” or “Y”
  VARIANT (3)..(3): /replace=“C” or “D” or “E” or “G” or “I” or “K” or “L” or “M” or “N” or “P” or “Q” or “R” or “T” or “V” or “W” or “Y”
  VARIANT (4)..(4): /replace=“Q” or “Y”
  VARIANT (5)..(5): /replace=“R” or “K”
  VARIANT (6)..(6): /replace=“E” or “L” or “M” or “N” or “Q” or “S” or “T” or “V”
  VARIANT (7)..(7): /replace=“C” or “D” or “E” or “F” or “G” or “I” or “K” or “L” or “M” or “N” or “P” or “Q” or “R” or “V” or “W” or “Y”
  VARIANT (8)..(8): /replace=“E” or “F” or “G” or “I” or “K” or “M” or “N” or “P” or “Q” or “R” or “T” or “V” or “W” or “Y”
  VARIANT (9)..(9): /replace=“D” or “E” or “F” or “G” or “H” or “I” or “K” or “L” or “M” or “N” or “Q” or “R” or “T” or “V” or “W” or “Y”
  SITE (1)..(9): /note=“Variant residues given in the sequence have no preference with respect to those in the annotations for variant positions”
AAAEGDAAC

SEQ ID NO: 780 - protein, 10 aa, Artificial Sequence
  source: /note=“Description of Artificial Sequence: Synthetic peptide”
  VARIANT (3)..(3): /replace=“C” or “D” or “E” or “F” or “G” or “H” or “I” or “K” or “L” or “M” or “N” or “P” or “Q” or “R” or “T” or “V” or “W” or “Y”
  VARIANT (4)..(4): /replace=“C” or “D” or “E” or “F” or “G” or “H” or “I” or “K” or “M” or “N” or “P” or “Q” or “R” or “S” or “T” or “V” or “W” or “Y”
  VARIANT (6)..(6): /replace=“G” or “H” or “I” or “K” or “L” or “M” or “P” or “Q” or “T” or “V” or “W” or “Y”
  VARIANT (7)..(7): /replace=“N” or “S”
  VARIANT (8)..(8): /replace=“C” or “D” or “E” or “F” or “H” or “K” or “L” or “M” or “N” or “P” or “Q” or “R” or “T” or “V”
  VARIANT (9)..(9): /replace=“C” or “D” or “E” or “F” or “G” or “H” or “K” or “M” or “N” or “P” or “Q” or “R” or “S” or “T” or “V” or “W” or “Y”
  VARIANT (10)..(10): /replace=“C” or “D” or “E” or “F” or “I” or “K” or “L” or “M” or “P” or “Q” or “R” or “T” or “V” or “W” or “Y”
  SITE (1)..(10): /note=“Variant residues given in the sequence have no preference with respect to those in the annotations for variant positions”
RGAAKFFAAA

SEQ ID NO: 781 - protein, 17 aa, Artificial Sequence
  source: /note=“Description of Artificial Sequence: Synthetic peptide”
  VARIANT (2)..(2): /replace=“C” or “D” or “E” or “G” or “H” or “I” or “K” or “L” or “M” or “N” or “P” or “Q” or “R” or “S” or “T” or “V” or “W” or “Y”
  VARIANT (3)..(3): /replace=“C” or “E” or “F” or “G” or “H” or “I” or “K” or “L” or “M” or “P” or “Q” or “R” or “S” or “V” or “Y”
  VARIANT (4)..(4): /replace=“D” or “E” or “F” or “G” or “H” or “I” or “K” or “L” or “M” or “N” or “Q” or “R” or “S” or “V”
  VARIANT (7)..(7): /replace=“C” or “D” or “E” or “F” or “G” or “H” or “I” or “K” or “L” or “M” or “N” or “P” or “Q” or “R” or “T” or “V” or “W” or “Y”
  VARIANT (8)..(8): /replace=“K” or “N” or “R” or “S” or “T”
  VARIANT (9)..(9): /replace=“D” or “E” or “F” or “G” or “H” or “K” or “L” or “M” or “N” or “P” or “Q” or “S” or “T” or “V” or “W” or “Y”
  VARIANT (11)..(11): /replace=“C” or “D” or “E” or “F” or “G” or “H” or “I” or “K” or “L” or “M” or “N” or “P” or “Q” or “R” or “T” or “V” or “W” or “Y”
  VARIANT (12)..(12): /replace=“C” or “D” or “E” or “F” or “H” or “I” or “K” or “L” or “M” or “N” or “P” or “Q” or “R” or “S” or “T” or “V” or “W” or “Y”
  VARIANT (13)..(13): /replace=“C” or “D” or “E” or “F” or “G” or “H” or “I” or “K” or “L” or “M” or “N” or “R” or “S” or “T” or “V” or “W” or “Y”
  VARIANT (15)..(15): /replace=“C” or “D” or “E” or “G” or “H” or “I” or “K” or “L” or “M” or “N” or “P” or “Q” or “R” or “S” or “T” or “V” or “W” or “Y”
  VARIANT (16)..(16): /replace=“C” or “D” or “F” or “G” or “H” or “I” or “K” or “L” or “M” or “N” or “P” or “S” or “T” or “V” or “W” or “Y”
  VARIANT (17)..(17): /replace=“C” or “E” or “F” or “G” or “H” or “I” or “K” or “L” or “M” or “N” or “P” or “Q” or “R” or “S” or “T” or “V” or “W” or “Y”
  SITE (1)..(17): /note=“Variant residues given in the sequence have no preference with respect to those in the annotations for variant positions”
  source: /note=“See specification as filed for detailed description of substitutions and preferred embodiments”
AAACMFAGCKAAANAAA

SEQ ID NO: 782 - protein, 15 aa, Artificial Sequence
  source: /note=“Description of Artificial Sequence: Synthetic peptide”
  VARIANT (1)..(1): /replace=“C” or “D” or “E” or “F” or “G” or “H” or “I” or “K” or “L” or “M” or “N” or “P” or “Q” or “R” or “S” or “T” or “V” or “W” or “Y”
  VARIANT (2)..(2): /replace=“G” or “H” or “I” or “T”
  VARIANT (3)..(3): /replace=“C” or “D” or “F” or “G” or “H” or “I” or “K” or “L” or “M” or “N” or “P” or “Q” or “R” or “S” or “T” or “V” or “W” or “Y”
  VARIANT (5)..(5): /replace=“H” or “I” or “L” or “M” or “N” or “Q” or “Y”
  VARIANT (6)..(6): /replace=“C” or “D” or “F” or “G” or “H” or “K” or “M” or “N” or “P” or “Q” or “R” or “S” or “T” or “V” or “Y”
  VARIANT (7)..(7): /replace=“C” or “E” or “F” or “H” or “K” or “M” or “N” or “P” or “Q” or “S” or “T” or “W” or “Y”
  VARIANT (8)..(8): /replace=“C” or “D” or “E” or “F” or “G” or “H” or “I” or “K” or “L” or “M” or “N” or “P” or “Q” or “S” or “T” or “V” or “W”
  VARIANT (9)..(9): /replace=“C” or “D” or “E” or “F” or “H” or “I” or “K” or “L” or “N” or “Q” or “R” or “S” or “V” or “W” or “Y”
  VARIANT (10)..(10): /replace=“C” or “D” or “E” or “F” or “H” or “I” or “K” or “L” or “M” or “N” or “P” or “Q” or “R” or “S” or “V” or “W” or “Y”
  VARIANT (11)..(11): /replace=“C” or “E” or “F” or “H” or “I” or “K” or “L” or “M” or “N” or “P” or “Q” or “S” or “T” or “V” or “W” or “Y”
  VARIANT (12)..(12): /replace=“Y”
  VARIANT (13)..(13): /replace=“C” or “D” or “E” or “F” or “G” or “H” or “I” or “K” or “L” or “N” or “P” or “Q” or “R” or “S” or “T” or “V” or “W” or “Y”
  VARIANT (15)..(15): /replace=“E” or “F” or “G” or “I” or “K” or “L” or “N” or “P” or “Q” or “R” or “S” or “T”
  SITE (1)..(15): /note=“Variant residues given in the sequence have no preference with respect to those in the annotations for variant positions”
AFADAAAAAAAFATD

SEQ ID NO: 783 - protein, 11 aa, Artificial Sequence
  source: /note=“Description of Artificial Sequence: Synthetic peptide”
  VARIANT (1)..(1): /replace=“C” or “D” or “E” or “F” or “G” or “H” or “I” or “L” or “M” or “N” or “P” or “R” or “S” or “T” or “V” or “W” or “Y”
  VARIANT (2)..(2): /replace=“C” or “D” or “E” or “F” or “G” or “H” or “I” or “K” or “L” or “M” or “N” or “Q” or “R” or “S” or “V” or “W” or “Y”
  VARIANT (3)..(3): /replace=“C” or “D” or “E” or “F” or “G” or “H” or “I” or “K” or “L” or “M” or “N” or “Q” or “S” or “T” or “V” or “W” or “Y”
  VARIANT (6)..(6): /replace=“C” or “D” or “E” or “F” or “G” or “H” or “K” or “M” or “N” or “P” or “Q” or “R” or “T” or “V” or “W” or “Y”
  VARIANT (7)..(7): /replace=“E” or “G” or “I” or “L” or “M” or “P” or “Q” or “V” or “W” or “Y”
  VARIANT (10)..(10): /replace=“C” or “D” or “E” or “F” or “G” or “H” or “K” or “L” or “M” or “N” or “P” or “Q” or “R” or “S” or “T” or “W” or “Y”
  SITE (1)..(11): /note=“Variant residues given in the sequence have no preference with respect to those in the annotations for variant positions”
AAAEDACPYAG

SEQ ID NO: 784 - protein, 12 aa, Artificial Sequence
  source: /note=“Description of Artificial Sequence: Synthetic peptide”
  VARIANT (1)..(1): /replace=“N” or “R”
  VARIANT (2)..(2): /replace=“C” or “D” or “E” or “G” or “H” or “K” or “M” or “N” or “Q” or “T” or “W”
  VARIANT (3)..(3): /replace=“C” or “D” or “E” or “F” or “G” or “H” or “K” or “L” or “M” or “N” or “P” or “Q” or “R” or “S” or “T” or “W” or “Y”
  VARIANT (6)..(6): /replace=“Q”
  VARIANT (7)..(7): /replace=“T” or “V”
  VARIANT (8)..(8): /replace=“D” or “E” or “G” or “H” or “I” or “K” or “L” or “M” or “P” or “Q” or “R” or “S” or “W” or “Y”
  VARIANT (10)..(10): /replace=“C” or “D” or “E” or “G” or “H” or “I” or “K” or “L” or “M” or “N” or “P” or “Q” or “T” or “W” or “Y”
  VARIANT (12)..(12): /replace=“I” or “L” or “P”
  SITE (1)..(12): /note=“Variant residues given in the sequence have no preference with respect to those in the annotations for variant positions”
  source: /note=“See specification as filed for detailed description of substitutions and preferred embodiments”
KAAVLHSCIARA

SEQ ID NO: 785 - protein, 9 aa, Artificial Sequence
  source: /note=“Description of Artificial Sequence: Synthetic peptide”
  VARIANT (1)..(1): /replace=“D” or “F” or “G” or “I” or “K” or “M” or “N” or “P” or “Q” or “S” or “T” or “V” or “W” or “Y”
  VARIANT (2)..(2): /replace=“C” or “D” or “F” or “G” or “I” or “L” or “M” or “N” or “P” or “Q” or “R” or “S” or “T” or “V” or “W”
  VARIANT (4)..(4): /replace=“H” or “L” or “M” or “P” or “Q” or “R” or “V” or “W”
  VARIANT (6)..(6): /replace=“N” or “P” or “R” or “W”
  VARIANT (7)..(7): /replace=“C” or “D” or “E” or “G” or “H” or “I” or “K” or “M” or “N” or “P” or “R” or “S” or “T” or “V” or “W” or “Y”
  VARIANT (8)..(8): /replace=“P” or “S” or “W”
  VARIANT (9)..(9): /replace=“D” or “E” or “F” or “G” or “H” or “K” or “L” or “M” or “Q” or “R” or “T” or “V” or “W” or “Y”
  SITE (1)..(9): /note=“Variant residues given in the sequence have no preference with respect to those in the annotations for variant positions”
CAPCFAADC

SEQ ID NO: 786 - protein, 5 aa, Artificial Sequence
  source: /note=“Description of Artificial Sequence: Synthetic peptide”
NYPMT

SEQ ID NO: 787 - protein, 17 aa, Artificial Sequence
  source: /note=“Description of Artificial Sequence: Synthetic peptide”
TIRGSGGDTWYADSVKG

SEQ ID NO: 788 - protein, 6 aa, Artificial Sequence
  source: /note=“Description of Artificial Sequence: Synthetic peptide”
WVGRDA

SEQ ID NO: 789 - protein, 16 aa, Artificial Sequence
  source: /note=“Description of Artificial Sequence: Synthetic peptide”
RSSQSLVYSDGNTYLN

SEQ ID NO: 790 - protein, 7 aa, Artificial Sequence
  source: /note=“Description of Artificial Sequence: Synthetic peptide”
KVSNRDS

SEQ ID NO: 791 - protein, 9 aa, Artificial Sequence
  source: /note=“Description of Artificial Sequence: Synthetic peptide”
MQGTHWPYT

SEQ ID NO: 792 - protein, 30 aa, Artificial Sequence; identical to SEQ ID NO: 87
  source: /note=“Description of Artificial Sequence: Synthetic polypeptide”

SEQ ID NO: 793 - protein, 14 aa, Artificial Sequence
  source: /note=“Description of Artificial Sequence: Synthetic peptide”
WVRQAPGKGLEWVS

SEQ ID NO: 794 - protein, 32 aa, Artificial Sequence; identical to SEQ ID NO: 265
  source: /note=“Description of Artificial Sequence: Synthetic polypeptide”

SEQ ID NO: 795 - protein, 11 aa, Artificial Sequence
  source: /note=“Description of Artificial Sequence: Synthetic peptide”
WGQGTLVTVSS

SEQ ID NO: 796 - protein, 23 aa, Artificial Sequence
  source: /note=“Description of Artificial Sequence: Synthetic peptide”
DIQLTQSPLSLPVTLGQPASISC

SEQ ID NO: 797 - protein, 15 aa, Artificial Sequence
  source: /note=“Description of Artificial Sequence: Synthetic peptide”
WFQQRPGQSPRRLIY

SEQ ID NO: 798 - protein, 32 aa, Artificial Sequence
  source: /note=“Description of Artificial Sequence: Synthetic polypeptide”
GVPDRFSGSVSGPDFTLKISRVEAEDVGVYYC

SEQ ID NO: 799 - protein, 13 aa, Artificial Sequence
  source: /note=“Description of Artificial Sequence: Synthetic peptide”
FGQGTKLEIKRTV

SEQ ID NO: 800 - protein, 115 aa, Artificial Sequence; identical to SEQ ID NO: 271
  source: /note=“Description of Artificial Sequence: Synthetic polypeptide”

SEQ ID NO: 801 - protein, 115 aa, Artificial Sequence
  source: /note=“Description of Artificial Sequence: Synthetic polypeptide”
DIQLTQSPLSLPVTLGQPASISCRSSQSLVYSDGNTYLNWFQQRPGQSPRRLIYKVSNRDSGVPDRFSGSVSGPDFTLKISRVEAEDVGVYYCMQGTHWPYTFGQGTKLEIKRTV

SEQ ID NO: 802 - protein, 115 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCARGRQVDKWGQGTLVTVSS

SEQ ID NO: 803 - protein, 110 aa, Artificial Sequence
  source: /note=“Description of Artificial Sequence: Synthetic polypeptide”
DIMLTQSPSSLSASVGDRVTITCRASQSIHNYLNWIQQTPGKAPKLLVFAASTLQRGVPPRYSGRGSGTEFTLTISSLQPEDFATYYCQQSYTTPLTFGGGTKVEIRRTV

SEQ ID NO: 804 - protein, 118 aa, Artificial Sequence
  source: /note=“Description of Artificial Sequence: Synthetic polypeptide”
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCAGGYSYSYMAIWGKGTTVTVSS

SEQ ID NO: 805 - protein, 110 aa, Artificial Sequence
  source: /note=“Description of Artificial Sequence: Synthetic polypeptide”
DIVLTQSPSSLSASVGDRVTITCRASQGISTFLNWYQQKPGKAPKLLIYAASSLQSGVPSRFSGSGSGTDFTLTIGSLQPEDFASYFCQQTYSTQWTFGQGTKVEIKRTV

SEQ ID NO: 806 - protein, 120 aa, Artificial Sequence
  source: /note=“Description of Artificial Sequence: Synthetic polypeptide”
QVQLVQSGAEVKRPGASVKVSCKASGYTFTNDIIHWVRQAPGQRLEWMGWINAGYGNTQYSQNFQDRVSITRDTSASTAYMELISLRSEDTAVYYCARGYYRSPTWFDWWGQGTLVTVSS

SEQ ID NO: 807 - protein, 110 aa, Artificial Sequence
  source: /note=“Description of Artificial Sequence: Synthetic polypeptide”
ETTLTQSPSSLSASVGDRVTITCRASQSISSWLAWYQQKPGKAPKLLIYKASSLESGVPSRFSGSGSGTEFTLTISSLQPDDFATYYCQQYGDYPYTFGQGTKLEIKRTV

SEQ ID NO: 808 - protein, 120 aa, Artificial Sequence; identical to SEQ ID NO: 669
  source: /note=“Description of Artificial Sequence: Synthetic polypeptide”

SEQ ID NO: 809 - protein, 110 aa, Artificial Sequence
  source: /note=“Description of Artificial Sequence: Synthetic polypeptide”
DIQLTQSPSSLSASVGDRVTITCQASQDISEYLNWYQHKPGKAPNPLVFDAANLETGVPPRFSATGSGTDFSLTITSLQPEDFATYYCQQYDSFPYTFGQGTRLDIKRTV

SEQ ID NO: 810 - protein, 119 aa, Artificial Sequence
  source: /note=“Description of Artificial Sequence: Synthetic polypeptide”
EVQLLESGGGLVQPGGSLRLSCAVSGFTFNSFAMSWVRQAPGKGLEWVSAISGSGGGTYYADSVKGRFTISRDNSKNTLYLQMNSLRAEDTAIYYCAREVDGYGAFDIWGQGTMVTVSS

SEQ ID NO: 811 - protein, 110 aa, Artificial Sequence
  source: /note=“Description of Artificial Sequence: Synthetic polypeptide”
DIQMTQSPSSLSASVGNRVTITCRASQTIRNYLNWYQQKPGEAPKVLIYGASTLQSGVPSRFSASGSGTDFTLTISSLQPDDFATYHCQQTFSDPYTFGQGTKVEIKRTV

SEQ ID NO: 812 - protein, 122 aa, Artificial Sequence
  source: /note=“Description of Artificial Sequence: Synthetic polypeptide”
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYYCAREGWEVRTPGYFDYWGQGTLVTVSS

SEQ ID NO: 813 - protein, 110 aa, Artificial Sequence
  source: /note=“Description of Artificial Sequence: Synthetic polypeptide”
DVVMTQSPSSLSASVGDRVTITCRASHRIFSYLTWYHQKPGKAPNLLISGASSLGAGVPSRFSGSGSGTDFTLTISSLQPEDFGTYYCQQSFSDPYTFGQGTKLEIKRTV

SEQ ID NO: 814 - protein, 115 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGITGSGGSTYYADSVKGRFTISRDNSKNTLYLQMNSLRAEDTAIYYCARGGALDIWGQGTMVTVSS

SEQ ID NO: 815 - protein, 110 aa, Artificial Sequence
  source: /note=“Description of Artificial Sequence: Synthetic polypeptide”
DIQMTQSPSSLSASVGDRVTITCRASQVIHNYLAWYKQKPGQAPKLLIYSASSLQSGVPSRFSGSGSGTDFTLTISSLQPEDFATYYCQQSYSTPLTFGGGTKVEIKRTV

SEQ ID NO: 816 - protein, 123 aa, Artificial Sequence
  source: /note=“Description of Artificial Sequence: Synthetic polypeptide”
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCAGRQYDFWSGWDWFDPWGQGTLVTVSS

SEQ ID NO: 817 - protein, 111 aa, Artificial Sequence
  source: /note=“Description of Artificial Sequence: Synthetic polypeptide”
DIQLTQSPSSLSASVGDRVTITCRASQSISTYLNWYQQKPGKAPKLLIHDASILQSGVPSRFSGSGSGTDFTLTISSLQAEDFATYYCQQSYSTLPYTFGQGTKVEIKRTV

SEQ ID NO: 818 - protein, 122 aa, Artificial Sequence; identical to SEQ ID NO: 703
  source: /note=“Description of Artificial Sequence: Synthetic polypeptide”

SEQ ID NO: 819 - protein, 110 aa, Artificial Sequence
  source: /note=“Description of Artificial Sequence: Synthetic polypeptide”
EIVLTQSPSSLSASVGDRVTISCRASRTVGTFLNWYQQRPGKPPQALILGVSILEGGVSSRFSGAGSGTEFSLTISSLQPEDIGTYYCQQSHDVPRTFGQGTKVDLRRTV

SEQ ID NO: 820 - protein, 115 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCARSRNLDSWGQGTLVTVSS

SEQ ID NO: 821 - protein, 110 aa, Artificial Sequence
  source: /note=“Description of Artificial Sequence: Synthetic polypeptide”
DIQLTQSPSSLSASVGDRVTITCRASQSIHNYLNWIQQTPGKAPKLLVFAASTLQRGVPPRYSGRGSGTEFTLTISSLQPEDFATYYCQQSYTTPLTFGGGTKVEIRRTV

SEQ ID NO: 822 - protein, 176 aa, Homo sapiens
QDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTH

SEQ ID NO: 823 - protein, 180 aa, Homo sapiens
LMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPH

SEQ ID NO: 824 - protein, 180 aa, Homo sapiens
HMGGQDKPFLSAWPSAVVPRGGHVTLRCHYRHRFNNFMLYKEDRIHIPIFHGRIFQESFNMSPVTTAHAGNYTCRGSHPHSPTGWSAPSNPVVIMVTGNHRKPSLLAHPGPLVKSGERVILQCWSDIMFEHFFLHKEGISKDPSRLVGQIHDGVSKANFSIGPMMLALAGTYRCYGSVTH

SEQ ID NO: 825 - protein, 85 aa, Homo sapiens
HEGVHRKPSLLAHPGPLVKSEETVILQCWSDVMFEHFLLHREGMFNDTLRLIGEHHDGVSKANFSISRMTQDLAGTYRCYGSVTH

SEQ ID NO: 826 - protein, 6 aa, Artificial Sequence
  source: /note=“Description of Artificial Sequence: Synthetic peptide”
VTYAQL

(19) United States
(12) Patent Application Publication
(10) Pub. No.: US 2023/0303695 A1
(43) Pub. Date: Sep. 28, 2023

(54) COMPOSITIONS AND METHODS FOR TREATING AUTOIMMUNE DISEASES AND CANCERS BY TARGETING IGSF8

(71) Applicants: Tengfei Xiao, Shanghai (CN); Xihao Hu, Sudbury, MA (US); Xiaole Shirley Liu, Weston, MA (US); SHANGHAI XBH BIOTECHNOLOGY CO., LTD., Shanghai (CN)

(72) Inventors: Tengfei Xiao, Shanghai (CN); Xihao Hu, Sudbury, MA (US); Xiaole Shirley Liu, Weston, MA (US)

(21) Appl. No.: 18/020,447

(22) PCT Filed: Aug. 9, 2021
(86) PCT No.: PCT/CN2021/111469

§ 371 Date: Feb. 9, 2023

(30) Foreign Application Priority Data
Aug. 10, 2020 (WO) PCT/CN2020/108129

(51) Int. Cl. C07K 16/28; C12N 15/63; G01N 33/68

(52) U.S. Cl. CPC C07K 16/2818; C12N 15/63; G01N 33/6854; C07K 2317/565; C07K 2317/24; C07K 2317/732; C07K 2317/734; C07K 2317/92

(57) ABSTRACT

The present invention provides methods and compositions for treating a cancer, and/or an autoimmune disease, by modulating the expression and/or activity of IGSF8 and its binding ligands. The pharmaceutical compositions may include, but are not limited to, antibodies that specifically bind human IGSF8, and have an activity of inhibiting IGSF8-mediated immunosuppression in a subject in need thereof.

### REFERENCE TO RELATED APPLICATION

This International Patent application claims priority to International Patent Application No. PCT/CN2020/108129, filed on Aug. 10, 2020, the entire contents of which, including all drawings and sequences, are incorporated herein by reference.

### BACKGROUND OF THE INVENTION

IGSF8 (Immunoglobulin Superfamily Member 8, also known as EWI-2, CD316, and numerous other aliases), encodes a 613-amino acid (or 65 kDa) protein that is a member of the EWI subfamily of the immunoglobulin protein superfamily. This subfamily of proteins all contain a single transmembrane domain, an EWI (Glu-Trp-Ile)-motif (hence the EWI subfamily), and a variable number of immunoglobulin domains.

Human and murine IGSF8 protein sequences are 91% identical. Although IGSF8 transcripts in the two species are expressed in virtually every tissue tested, little is known about the biological function of IGSF8. It has been reported that IGSF8 specifically and directly interacts with the tetraspanins CD81 and CD9 but not with other tetraspanins or with integrins, and it is speculated to regulate the roles of CD9 and CD81 in certain cellular functions, including cell migration and viral infection (Stipp et al., J. Biol. Chem. 276(44):40545-40554, 2001). IGSF8 has also been identified as a potential tumor suppressor, because it has been found to directly interact with another tetraspanin KAI1/CD82, a cancer metastasis suppressor. It has been speculated that IGSF8 is important or likely required for KAI1/CD82-mediated suppression of cancer cell migration (Zhang et al., Cancer Res. 63(10):2665-2674, 2003). IGSF8 has also been found to bind to integrin α4β1 from MOLT-4 T leukemia cells, and it has been suggested that IGSF8-dependent reorganization of α4β1-CD81 complexes on the cell surface is responsible for IGSF8 effects on integrin-dependent morphology and motility functions (Kolesnikova et al., Blood 103(8):3013-3019, 2004). Lastly, IGSF8 has been found to regulate α3β1 integrin-dependent cell function on laminin-5 (Stipp et al., JCB 163(5):1167-1177, 2003).

Although remarkable clinical benefits derived from checkpoint-based immunotherapy, such as using anti-CTLA-4 and anti-PD-1/PD-L1 antibodies, have been noted in many patients, there are still a majority of cancer patients who do not respond to these treatments. Researchers are trying to understand why such T cell-based immunotherapies are not effective in these so-called “non-responders.”

Tumors can escape T cell-mediated immunity by down-regulating the expression of major histocompatibility complex class I (MHC-I) molecules. Partial or complete loss of MHC-I expression on the surface of cancer cells has been demonstrated to be a major mechanism of acquired resistance to certain T cell-based immunotherapy. More importantly, about 40% of cancer patients who had acquired resistant to anti-PD-1/PD-L1 or CTLA4 immunotherapy showed total loss of MHC-I expression on their cancer cells. These tumors are “immune-cold” tumors, which unfortunately constitute more than 70% of all tumors in cancer patients.

Although MHC-I-null tumor cells can completely evade killing by T cells, at least in theory, they are still susceptible to destruction by natural killer (NK) cells of the innate immune system. However, in the tumor microenvironment (TME), for reasons not yet fully understood, most NK cells are inactivated, and are not able to specifically recognize or kill cancer cells without MHC-I expression.

Meanwhile, certain immuno-inhibitory receptors (e.g., NKG2A, PD-1, LAG-3, TIGIT, and TIM-3) have bene found to express on both effector T/NK cells. Some monoclonal antibodies raised against these targets were able to reverse the functional exhaustion of NK cells in the tumors, thus raising the hope that NK cell-based cancer immunotherapy may complement the limitations of T cell-based immunotherapy. However, almost all ligands on cancer cells that have been identified to be able to suppress NK cell activity in the tumor microenvironment are HLA ligands, which are extremely diverse from one individual to another unrelated individual, raising the doubt that such strategy may not be generally applicable to the larger patient population. Meanwhile, very few non-HLA ligands on cancers cells has been identified to be able to suppress NK cell activity in the tumor microenvironment.

Thus, there remains a need to identify NK cell-suppressing, non-HLA ligands that may have been hijacked by cancer cells to evade NK cell-mediated killing in the tumor microenvironment, and reagents that can block suppression of NK cells, in order to facilitate NK cell-based cancer immunotherapy.

### SUMMARY OF THE INVENTION

One aspect of the invention provides isolated or recombinant monoclonal antibody or an antigen-binding fragment thereof specific for IGSF8 (e.g., specific for the Ig-V set domain or the D1 domain of the ECD of IGSF8), wherein said monoclonal antibody or antigen-binding fragment thereof comprises a heavy chain variable region (VH) comprising a VH CDR1, a VH CDR2, and a VH CDR3, and a light chain variable region (VL) comprising a VL CDR1, a VL CDR2 and a VL CDR3, wherein (a1) the VH CDR1, VH CDR2 and VH CDR3 comprise, consists essentially of, or consists of the amino acid sequence of SEQ ID NOs: 714, 715 and 716, respectively; and the VL CDR1, VL CDR2 and VL CDR3 comprise, consists essentially of, or consists of the amino acid sequence of SEQ ID NOs: 717, 718 and 719, respectively; or (a2) the VH CDR1, VH CDR2 and VH CDR3 comprise, consists essentially of, or consists of the amino acid sequence of SEQ ID NOs: 754, 755 and 756, respectively; and the VL CDR1, VL CDR2 and VL CDR3 comprise, consists essentially of, or consists of the amino acid sequence of SEQ ID NOs: 757, 758 and 759, respectively; or (b1) the VH CDR1, VH CDR2 and VH CDR3 comprise, consists essentially of, or consists of the amino acid sequence of SEQ ID NOs: 720, 721 and 722, respectively; and the VL CDR1, VL CDR2 and VL CDR3 comprise, consists essentially of, or consists of the amino acid sequence of SEQ ID NOs: 723, 724 and 725, respectively; or (b2) the VH CDR1, VH CDR2 and VH CDR3 comprise, consists essentially of, or consists of the amino acid sequence of SEQ ID NOs: 760, 761 and 762, respectively; and the VL CDR1, VL CDR2 and VL CDR3 comprise, consists essentially of, or consists of the amino acid sequence of SEQ ID NOs: 763, 764 and 765, respectively; or (c) the VH CDR1, VH CDR2 and VH CDR3 comprise, consists essentially of, or consists of the amino acid sequence of any of the VH CDR1, VH CDR2 and VH CDR3 sequences, respectively, of Table D and Table G; and the VL CDR1, VL CDR2 and VL CDR3 comprise, consists essentially of, or consists of the amino acid sequence of any of the VL CDR1, VL CDR2 and VL CDR3 sequences, respectively, of Table D and Table G; or (d) the VH CDR1, VH CDR2, VH CDR3, VL CDR1, VL CDR2, and VL CDR3 comprise, consists essentially of, or consists of the amino acid sequence of the VH CDR1, VH CDR2 VH CDR3, VL CDR1, VL CDR2, and VL CDR3 sequences, respectively, of any one antibody of Table D and Table G; optionally, the antibody and the antigen-binding fragment thereof do not have the same VH CDR1, VH CDR2 VH CDR3, VL CDR1, VL CDR2, and VL CDR3 sequences of the L1 antibody and that of the L2 antibody (e.g., the the antibody is not L1 and is not L2).

In certain embodiments, the monoclonal antibody or an antigen-binding fragment thereof comprises (1) VH CDR1, VH CDR2, VH CDR3, VL CDR1, VL CDR2, and VL CDR3 comprising the amino acid sequences of the VH CDR1, VH CDR2 VH CDR3, VL CDR1, VL CDR2, and VL CDR3 sequences, respectively, of any one antibody of Table D; or (2) VH CDR1, VH CDR2, VH CDR3, VL CDR1, VL CDR2, and VL CDR3 comprising the amino acid sequences of the VH CDR1, VH CDR2 VH CDR3, VL CDR1, VL CDR2, and VL CDR3 sequences, respectively, of any one antibody of Table G.

In some embodiments, the monoclonal antibody or an antigen-binding fragment thereof comprises a VH and VL, wherein (a) the VH comprising a VH FR1, a VH FR2, a VH FR3, and/or a VH FR4 comprising (i) the amino acid sequence(s) of the corresponding VH FR sequence(s) of any one or more antibodies in Table D (or Table G), (ii) an amino acid sequence having at least 90%, 91%, 92%, 93%, 94%, 95%, 96%, 97%, 98%, 99% sequence identity to the corresponding VH FR sequence(s) of any one or more antibodies in Table D (or Table G); or (iii) an amino acid sequence having at most 1, 2, 3, 4, or 5 substitutions, deletions, and/or additions compared to the corresponding VH FR sequence(s) of any one or more antibodies in Table D (or Table G); and/or (b) the VL comprises a VL FR1, a VL FR2, a VL FR3, and/or a VL FR4 comprising (i) the amino acid sequence(s) of the corresponding VL FR sequence(s) of any one or more antibodies in Table D (or Table G), (ii) an amino acid sequence having at least 90%, 91%, 92%, 93%, 94%, 95%, 96%, 97%, 98%, 99% sequence identity to the corresponding VL FR sequence(s) of any one or more antibodies in Table D (or Table G); or (iii) an amino acid sequence having at most 1, 2, 3, 4, or 5 substitutions, deletions, and/or additions compared to the corresponding VL FR sequence(s) of any one or more antibodies in Table D (or Table G).

In some embodiments, the monoclonal antibody or an antigen-binding fragment thereof comprises VH and VL, wherein (a1) the VH comprises the amino acid sequence of SEQ ID NOs: 734, 735 and 736, respectively; and the VL comprises the amino acid sequence of SEQ ID NOs: 737, 738 and 739, respectively; or (a2) the VH comprises the amino acid sequence of SEQ ID NOs: 774, 775 and 776, respectively; and the VL comprises the amino acid sequence of SEQ ID NOs: 777, 778 and 779, respectively; or (1)1) the VH comprises the amino acid sequence of SEQ ID NOs: 740, 741 and 742, respectively; and the VL comprises the amino acid sequence of SEQ ID NOs: 743, 744 and 745, respectively; or (b2) the VH comprises the amino acid sequence of SEQ ID NOs: 780, 781 and 782, respectively; and the VL comprises the amino acid sequence of SEQ ID NOs: 783, 784 and 785, respectively; or (c) the VH comprises the amino acid sequence of any VH sequence of Table D and Table G; and the VL comprises the amino acid sequence of any VL sequence of Table D and Table G.

In some embodiments, the VH and VL seqeuences comprise the amino acid sequences of the VH and VL sequences, respectively, of any one antibody of Table D and Table G.

In some embodiments, the monoclonal antibody or antigen-binding fragment thereof is a human-mouse chimeric antibody, a humanized antibody, a human antibody, a CDR-grafted antibody, or a resurfaced antibody.

In some embodiments, said antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In some embodiments, the monoclonal antibody or antigen-binding fragment thereof comprises a heavy chain constant region, wherein (a) the heavy chain constant region is wild-type human IgG1, human IgG2, human IgG3, human IgG4; or (b) the heavy chain constant region has an Fc domain deficient in antibody-dependent cellular cytotoxicity (ADCC), complement-dependent cytotoxicity (CDC) and/or antibody-dependent cellular phagocytosis (ADCP).

In some embodiments, the heavy chain constant region with an deficient Fc domain is selected from a group consisting of IgG1-L234A/L235A (IgG1-LALA), IgG1-L234A/L235A/P329G (IgG1-LALA-PG), IgG1-N297A/Q/G (IgG1-NA), IgG1-L235A/G237A/E318A (IgG1-AAA), IgG1-G236R/L328R (IgG1-RR), IgG1-S298G/T299A (IgG1-GA), IgG1-L234F/L235E/P331S (IgG1-FES), IgG1-L234F/L235E/D265A (IgG1-FEA), IgG4-L234A/L235A (IgG4-LALA), IgG4-S228P/L235E (IgG4-PE), IgG1-E233P/L234V/L235A/G236del/S267K, IgG2-H268Q/V309L/A30S/P331S (IgG2m4) and IgG2-V234A/G237A/P238S/H268A/V309L/A330S/P331S (IgG2c4d).

In some embodiments, said monoclonal antibody or antigen-binding fragment thereof binds IGSF8 with a K_{d} of less than about 25 nM, 20 nM, 15 nM, 10 nM, 5 nM, 2 nM, or 1 nM.

In certain aspects, the invention provides a monoclonal antibody or an antigen-binding fragment thereof, which competes with the monoclonal antibody or antigen-binding fragment thereof of the invention for binding to IGSF8.

Another aspect of the invention provides a monoclonal antibody or an antigen-binding fragment thereof specific for IGSF8, wherein the monoclonal antibody comprises: (1) a heavy chain variable region (HCVR), comprising HCVR CDR1-CDR3 sequences at least 95% (e.g., 100%) identical to, or having up to 1, 2, 3, 4, 5, 6, 7, 8, or 9 substitutions in HCVR CDR1-CDR3, respectively, of any one of antibodies C1-C39, such as C30-C39; and, (2) a light chain variable region (LCVR), comprising LCVR CDR1-CDR3 sequences at least 95% (e.g., 100%) identical to, or having up to 1, 2, 3, 4, 5, 6, 7, 8, or 9 substitutions in LCVR CDR1-CDR3, respectively, of any one of antibodies C1-C39, such as C30-C39.

A related aspect of the invention provides a monoclonal antibody or an antigen-binding fragment thereof, which competes with the monoclonal antibody or antigen-binding fragment thereof of the invention for binding to IGSF8.

In yet another related aspect, the invention provides a monoclonal antibody or an antigen-binding portion/fragment thereof, which specifically binds the D1 ECD (or Ig-V set domain) of IGSF8, and inhibits binding to KIR3DL1/2, such as binding to the D2 domain of KIR3DL1/2 (e.g., an epitope comprising S165, I171, and/or M186 of KIR3DL1/2).

Another aspect of the invention provides a polynucleotide encoding a monoclonal antibody of the invention, a heavy chain or a light chain thereof, or an antigen-binding portion/fragment thereof.

In a related aspect, the invention provides a polynucleotide that hybridizes under stringent conditions with the polynucleotide of the invention or a complement thereof.

Another aspect of the invention provides a vector comprising the polynucleotide of the invention.

Another aspect of the invention provides a host cell comprising the polynucleotide of the invention, or the vector of the invention, for expressing the encoded monoclonal antibody of the invention, heavy or light chain thereof, or antigen-binding portion/fragment thereof.

Another aspect of the invention provides a method of producing the monoclonal antibody of the invention, heavy or light chain thereof, or antigen-binding portion/fragment thereof of the invention, the method comprising: (i) culturing the host cell of the invention capable of expressing the monoclonal antibody of the invention, heavy or light chain thereof, or antigen-binding portion/fragment thereof under a condition suitable to express the monoclonal antibody, heavy or light chain thereof, or antigen-binding portion/fragment thereof; and (ii) recovering/isolating/purifying the expressed monoclonal antibody of the invention, heavy or light chain thereof, or antigen-binding portion/fragment thereof.

Another aspect of the invention provides a method of modulating an immune response in a subject in need thereof, the method comprising inhibiting interaction between IGSF8 and a receptor of IGSF8 selected from KIR3DL1, KIR3DL2, and KLRC1/D2 heterodimer.

Another aspect of the invention provides a method of immunotherapy for treating a cancer in a subject in need thereof, the method comprising inhibiting interaction between IGSF8 and a receptor of IGSF8 selected from KIR3DL1, KIR3DL2, and KLRC1/D2 heterodimer.

Another aspect of the invention provides a method of treating cancer in a subject in need thereof, the method comprising administering to the subject a therapeutically effective amount of an IGSF8 (Immuno Globulin Super Family 8) modulator (e.g., antagonist).

Another aspect of the invention provides a method of treating cancer in a subject in need thereof, the method comprising administering to the subject a therapeutically effective amount of an KIR3DL1 antagonist that inhibits interaction with IGSF8.

Another aspect of the invention provides a method of treating cancer in a subject in need thereof, the method comprising administering to the subject a therapeutically effective amount of an KIR3DL2 antagonist that inhibits interaction with IGSF8.

Another aspect of the invention provides a method of treating cancer in a subject in need thereof, the method comprising administering to the subject a therapeutically effective amount of an KLRC1/D1 antagonist that inhibits interaction with IGSF8.

Another aspect of the invention provides a use of an IGSF8 antagonist, an KIR3DL1 antagonist, an KIR3DL2 antagonist, or an KLRC1/D1 antagonist that inhibits IGSF8 binding to a receptor of IGSF8 selected from KIR3DL1, KIR3DL2, and KLRC1/D2 heterodimer, for treating cancer in a subject.

Another aspect of the invention provides a composition comprising an IGSF8 antagonist, an KIR3DL1 antagonist, an KIR3DL2 antagonist, or an KLRC1/D1 antagonist, that inhibits IGSF8 binding to a receptor of IGSF8 selected from KIR3DL1, KIR3DL2, and KLRC1/D2 heterodimer, for use in any of the preceding method claims.

Another aspect of the invention provides an antibody which specifically bind IGSF8 for use in a method of treating cancer, preferably through stimulating T cell and/or NK cell activation.

Another aspect of the invention provides an antibody which specifically bind IGSF8 for use in a method of treating cancer, preferably through combination with a second therapeutic agent as described herein, such as checkpoint inhibitor-mediated immune therapy.

Another aspect of the invention provides a device or kit comprising at least one antibody, monoclonal antibody, heavy or light chain thereof, or antigen-binding portion/fragment thereof, of the invention, the device or kit optionally comprising a label to detect said at least one antibody, monoclonal antibody, heavy or light chain thereof, or antigen-binding portion/fragment thereof, or a complex comprising said at least one antibody, monoclonal antibody, heavy or light chain thereof, or antigen-binding portion/fragment thereof.

Another aspect of the invention provides a fusion protein comprising an IGSF8 polypeptide and an antibody Fc region.

Another aspect of the invention provides a polynucleotide encoding the fusion protein of the invention.

Another aspect of the invention provides a vector comprising the polynucleotide encoding the fusion protein of the invention.

Another aspect of the invention provides a host cell comprising the polynucleotide encoding the fusion protein of the invention, or the vector comprising the polynucleotide encoding the fusion protein of the invention, for expressing the encoded fusion protein.

Another aspect of the invention provides a method of producing the fusion protein of the invention, the method comprising: (i) culturing the host cell of the invention capable of expressing said fusion protein under a condition suitable to express said fusion protein; and (ii) recovering/isolating/purifying the expressed fusion protein.

Another aspect of the invention provides a method of suppressing activity of a primary NK cell or a T cell, comprising contacting said primary NK cell or said T cell with the fusion protein of the invention.

Another aspect of the invention provides a method of detecting the presence or level of an IGSF8 polypeptide in a sample, the method comprising contacting the IGSF8 polypeptide in the sample with the antibody, monoclonal antibody, or antigen-binding portion/fragment thereof, of the invention, wherein said antibody, monoclonal antibody, or antigen-binding portion/fragment thereof is labeled by a detectable label, or can be attached to a detectable label.

Another aspect of the invention provides a method for monitoring the progression of a disorder associated with aberrant (e.g., higher than normal) IGSF8 expression in a subject, the method comprising: a) detecting, in a sample obtained from the subject, at a first point in time a first level of IGSF8 using the antibody, monoclonal antibody, or antigen-binding portion/fragment thereof, of the invention; b) repeating step a) at a subsequent point in time to obtain a second level of IGSF8; and c) comparing the first and the second levels of IGSF8 detected in steps a) and b), respectively, to monitor the progression of the disorder in the subject, wherein a higher second level than the first level is indicative that the disease has progressed.

Another aspect of the invention provides a method for predicting the clinical outcome of a subject afflicted with a disorder associated with aberrant (e.g., higher than normal) IGSF8 expression, the method comprising: a) determining the level of IGSF8 in a first sample obtained from the subject, using the antibody, monoclonal antibody, or antigen-binding portion/fragment thereof, of the invention; b) determining the level of IGSF8 in a second sample obtained from a control subject having a good clinical outcome, using the antibody, monoclonal antibody, or antigen-binding portion/fragment thereof, of the invention; and c) comparing the level of IGSF8 in the first and the second samples; wherein a significantly higher (e.g., >20%, >50% or more increase) level of IGSF8 in the first sample as compared to the level of IGSF8 in the second sample is an indication that the subject has a worse clinical outcome, and/or, wherein a significantly lower (e.g., >20%, >50% or more decrease) level of IGSF8 in the first sample as compared to the level of IGSF8 in the second sample is an indication that the subject has a better clinical outcome.

Another aspect of the invention provides a method of assessing the efficacy of a therapy for a disorder associated with aberrant (e.g., higher than normal) IGSF8 expression in a subject, the method comprising: a) determining the level of IGSF8 using the antibody, monoclonal antibody, or antigen-binding portion/fragment thereof, of the invention, in a first sample obtained from the subject prior to providing at least a portion of the therapy to the subject, and b) repeat step a) in a second sample obtained from the subject following provision of said portion of the therapy, wherein a significantly lower (>20%, >50% or more decrease) level of IGSF8 in the second sample, relative to the first sample, is an indication that the therapy is efficacious for inhibiting the disorder in the subject; and/or, wherein a substantially identical or higher level of IGSF8 in the second sample, relative to the first sample, is an indication that the therapy is not efficacious for inhibiting the disorder in the subject.

Another aspect of the invention provides a method of assessing the efficacy of a test compound for inhibiting a disorder associated with aberrant (e.g., higher than normal) IGSF8 expression in a subject, the method comprising: a) determining the level of IGSF8 using the antibody, monoclonal antibody, or antigen-binding portion/fragment thereof, of the invention, in a first sample obtained from the subject, wherein the first sample has been exposed to an amount of the test compound; and b) determining the level of IGSF8 using the antibody, monoclonal antibody, or antigen-binding portion/fragment thereof, of the invention, in a second sample obtained from the subject, wherein the second sample has not been exposed to the test compound, wherein a significantly lower (>20%, >50% or more decrease) level of IGSF8 in the first sample relative to that of the second sample, is an indication that the amount of the test compound is efficacious for inhibiting the disorder in the subject, and/or, wherein a substantially identical level of IGSF8 in the first sample relative to that of the second sample, is an indication that the amount of the test compound is not efficacious for inhibiting the disorder in the subject.

Another aspect of the invention provides a method of screening for a functional IGSF8 antagonist, the method comprising contacting a candidate agent (e.g., small molecule, peptide, aptamer, polynucleotide, etc) with a co-culture of NK cells and target cells that express IGSF8 and are resistant to NK cell-mediated cytotoxicity, and identifying the candidate agent that promotes NK cell-mediated cytolytic activity towards the target cell, thereby identifying the candidate agent as an IGSF8 antagonist.

Another aspect of the invention provides a method of screening for a functional IGSF8 antagonist, the method comprising contacting a candidate agent (e.g., small molecule, peptide, aptamer, polynucleotide, etc) with a Jurkat NFAT reporter cell in the presence of T-cell activation signals and IGSF8, wherein the candidate agent is identified as the functional IGSF8 antagonist, when the reporter cell is not activated in the absence of the candidate agent and is activated in the presence of the candidate agent.

Another aspect of the invention provides an antibody which specifically bind KIR3DL1/2 for use in a method of treating cancer, through inhibiting KIR3DL1/2-IGSF8 interaction, thereby stimulating NK cell activation.

Another aspect of the invention provides an antibody which specifically bind KIR3DL1/2 for use in a method of treating cancer, preferably through combination with a second therapeutic agent of the invention as described herein, such as a checkpoint inhibitor-mediated immune therapy.

Another aspect of the invention provides a monoclonal antibody or an antigen-binding fragment thereof specific for KIR3DL1/2, preferably the second/middle/D2 Ig-like domain of the ECD of KIR3DL1/2, or an epitope comprising residues S165, I171, and/or M186.

Another aspect of the invention provides a monoclonal antibody or an antigen-binding fragment thereof, which competes with the monoclonal antibody or antigen-binding fragment thereof for binding to KIR3DL1/2.

Another aspect of the invention provides a monoclonal antibody or an antigen-binding portion/fragment thereof, which specifically binds the middle/D2 ECD of KIR3DL1/2 (e.g., specifically binds an epitope comprising residues S165, I171, and/or M186), which inhibits IGSF8 binding to KIR3DL1/2.

It should be understood that any one embodiment of the invention, including those embodiments described only in the Examples or claims, can be freely combined with any other one or more additional embodiments of the invention, unless expressly and explicitly excluded or being improper.

### BRIEF DESCRIPTION OF THE DRAWINGS

FIG. 1 shows results of a genome-wide natural killer (NK) cell and cancer cell line (colorectal cancer cell line Colo205) co-culture screen, demonstrating that loss of IGSF8 function in Colo205 enhances natural killer (NK) cell cytotoxicity against Colo205. IGSF8 gene is the top 2 hits whose loss sensitized Colo205 cell killing by NK cells.

FIG. 2A shows dose response curves of primary NK cells from human Donor 2 and human Donor 3 treated with human Fc control, or human IGSF8-hFc (human Fc tagged IGSF8). Compared to the Fc control, NK cell viability is significantly reduced as concentration of IGSF8-hFc increases.

FIG. 2B shows dose response curves of primary T cells from human Donor 2 treated with human Fc (hFc) control, or human IGSF8-hFc (human Fc tagged IGSF8). Compared to the hFc control, T cell viability is significantly reduced as concentration of IGSF8-hFc increases.

FIG. 2C confirms the statistically significant (p<0.005) reduction of NK cell viability by IGSF8-Fc fusion protein in a dose-dependent manner.

FIG. 2D shows the top five enriched KEGG pathways down-regulated in the RNA-seq of NK cells treated with IGSF8-hFc fusion protein or hFc control protein.

FIG. 2E shows the relative mRNA expression of the genes in NK cells treated with IGSF8-hFc fusion protein or hFc control protein.

FIG. 2F shows effect of IGSF8-hFc fusion proteins on primary NK cell proliferation.

FIG. 2G shows effect of IGSF8-hFc fusion proteins on primary CD⁴⁺ T cell proliferation.

FIG. 2H shows effect of IGSF8-hFc fusion proteins on primary CD⁴⁺ T cell activation.

FIG. 3A shows that CRISPR/Cas9-mediated IGSF8 deletion in the B16-F10 melanoma cells significantly (p<0.0001) reduces the ability of such tumor cells to grow in vivo (as measured by tumor volume in mm³) in a mouse xenograph model (n=8 mice per group). sg IGSF8-1 and -2 represent two experimental groups in which IGSF8 gene was deleted in B16-F10 tumor cells, using two different CRISPR/Cas9 sgRNAs targeting different regions of IGSF8, prior to injection of these IGSF8-deleted B16-F10 tumors into the mice. As a control, the AAV integration site AAVS1 has been deleted similarly in the control B16-F10 tumor cells using sgRNA specific for AAVS1.

FIG. 3B shows that retarded tumor growth in vivo after IGSF8 deletion is not due to difference in relative in vitro cell growth rate of gene-deleted B16-F10 melanoma cells. There is no statistically significant difference in in vitro cell growth rate among the B16-F10 cells deleted of IGSF8, and B16-F10 cells deleted of AAVS1.

FIG. 4 shows that deletion of IGSF8 via CRISPR/Cas9-mediated gene editing in a varieties of cancer cell lines promote CXCL10 expression, which was measured as relative expression fold increase for CXCL10 compared to the same cancer cells deleted of AAVS1. H292 (NCI-H292) is a human mucoepidermoid pulmonary carcinoma cell line; A549 is a human lung carcinoma cell line; Colo205 is a Dukes' type D, colorectal adenocarcinoma cell line; N87 is a human gastric carcinoma cell line; and A375 is a human melanoma cell line.

FIGS. 5A-5D show enhanced relative expression of a varieties of genes in B16-F10 cells (FIGS. 5A and 5C) and tumors (FIGS. 5B and 5D), upon deletion of AAVS1 or IGSF8 by CRISPR/Cas9-mediated gene editing. *: P<0.05; **: P<0.01; ***: P<0.001.

FIG. 6A shows gene expression of IGSF8 in human cancer cell lines (date obtained from the Broad Institute Cancer Cell Line Encyclopedia (CCLE).

FIG. 6B shows statistically significantly elevated expression of IGSF8 in various tumors in The Cancer Genome Atlas (TCGA) cohorts.

FIG. 6C shows clinical relevance of IGSF8 in The Cancer Genome Atlas (TCGA) cohorts. Higher expression of IGSF8 is associated with worse clinical outcome in different cancer types.

FIG. 7 shows binding affinities of representative recombinant anti-IGSF8 antibodies of the invention for the IGSF8 extracellular domain, and EC50 values thereof measured by ELISA.

FIG. 8 shows antibody-dependent cellular cytotoxicity (ADCC) assay and the associated EC50 values for representative anti-IGSF8 antibodies of the invention, using NK cells as effector cells, and A431 cancer cells as target cells.

FIG. 9 shows human CXCL10 ELISA assay for Colo205 cells treated with representative anti-IGSF8 antibodies of the invention (10m/mL).

FIG. 10 shows effects of representative anti-IGSF8 monoclonal antibodies of the invention on tumor growth in B16 syngeneic mice. B16-F10 cells were injected subcutaneously into wild type (WT) C57BL/6 mice. Mice were then treated with 2 mg/kg anti-IGSF8 antibodies or control human IgG1 from day 6, every 3 days, for four doses in total. Data are presented as mean±S.E.M. (n=8 mice per group).

FIG. 11 is a line graph showing no significant weight difference among groups of the experimental mice treated with anti-IGSF8 antibodies, or with control human IgG1.

FIG. 12 shows synergistic effect between a subject anti-IGSF8 antibody and an anti-PD-1 antibody in reducing B16-F10 melanoma tumor volume increase in syngeneic mice.

FIG. 13A shows the effect of IGSF8-hFc fusion proteins on cytolytic activity of NK cells co-cultured with K562 cells.

FIG. 13B shows the effect of IGSF8-hFc fusion proteins on perforin production of NK cells in a NK-K562 co-culture model.

FIG. 14 shows the effect on cytolytic activity of NK cells co-cultured with K562 cells, K562 cells with forced expression of IGSF8, or IGSF8 knockout K562 cells. The NK cells were from two different donors.

FIG. 15A shows the topological domain of IGSF8.

FIG. 15B shows the effect of D1 and D2-4 domains of IGSF8 proteins on cytolytic activity of NK cells co-cultured with K562 cells.

FIG. 16A shows the outline of the CRISPR screen strategy for de-orphaning receptors of IGSF8 on NK cells.

FIG. 16B shows the dot plot of top selected genes from the CRISPR screen.

FIG. 17A shows a core map of a lentivrial vector used to express KIR receptors.

FIG. 17B shows binding of biotin-labelled IGSF8 to different KIR family proteins.

FIG. 17C shows a core map of two lentivrial vectors used to express the KLRC1/D1 heterodimeric receptors.

FIG. 17D shows that only the KLRC1/D1 heterodimers, not each monomers alone, bind the recombinant IGSF8-hFc protein.

FIG. 17E shows that IGSF8 binding to the KIR3D1/2 or the KLRC1/D1 receptors is mediated by the D1 (Ig-V set) ECD of IGSF8.

FIG. 18A shows the topological domain of KIR3DL1/2, as well as the individual domain constructs used to narrow down the binding domain of KIR3DL1/2 for IGSF8.

FIG. 18B shows binding of biotin-labelled IGSF8 to different domains of KIR3DL1/2.

FIG. 19A shows multiple sequence alignment of KIR family proteins, and the three residues required for IGSF8 binding.

FIG. 19B shows crystal structure of KIR3DL1, and the three residues required for IGSF8 binding.

FIG. 20 shows binding of biotin-labelled IGSF8 to different mutants of KIR3DL1/2.

FIG. 21 shows binding and EC50 values of IGSF8 monoclonal antibodies (mAbs) B34, 1B4, 2B4, 1C2, 3F12, B46, and B104 to CT26 cells with forced cell surface-expression of human IGSF8. At least a few of these antibodies (e.g., 1B4, B46, and B104) also bind mouse IGSF8 expressed on CT26 cells (data not shown).

FIG. 22 shows binding of IGSF8 mAbs to the D1 domain of IGSF8 on CT26 cells.

FIG. 23A is a diagram of two embodiments of an antibody blocking assay. In the left panel, CT26 cells expressing ligand IGSF8 are treated with soluble and biotin labelled receptor (KIR3DL1/2) and anti-IGSF8 mAbs, and subsequently, bound receptor is detected with PE-streptavidin. In the right panel, MC38 cells expressing the IGSF8 ligand are contacted with KLR- or KIR-receptor-expressing CT26 cells, and anti-IGSF8 antibodies capable of blocking MC38-CT26 cell/cell conjugates will reduce the formation of the FACS-detectable conjugate.

FIG. 23B shows the blocking of cell-cell conjugate formation between IGSF8-expressing MC38 cells and the KIR3DL2-expressing CT26 cells by selected anti-IGSF8 antibodies.

FIG. 23C shows the blocking of cell-cell conjugate formation between IGSF8-expressing MC38 cells and the KLRC1/D1 heterodimer-expressing CT26 cells by anti-IGSF8 antibodies.

FIG. 24A is a diagram of NK cell suppression assay in FIG. 24B.

FIG. 24B shows that IGSF8-mediated suppression of K562 cell killing by human primary NK cells can be reversed by anti-IGSF8 mAbs.

FIG. 25A shows in vivo anti-tumor efficacy using B16-F10 syngeneic model.

FIG. 25B shows response of individual mice treated with anti-IGSF8 mAb or isotype-matched IgG control.

FIG. 26A shows in vivo anti-tumor efficacy using LLC syngeneic mouse model.

FIG. 26B shows in vivo anti-tumor efficacy using CT26 syngeneic mouse model.

FIG. 27 shows relative mRNA expression of the genes in LLC syngeneic mouse model.

FIG. 28 shows the amino acid sequences of the heavy and light chain variable region of L1 and L2 antibodies. CDR sequences according to the IMGT numbering scheme are in boxs. Underlined sequences include CDR regions as well as neighboring framework region sequences that may affect binding affinity.

FIG. 29 shows the heatmap of negative selection of the mutants within the L1 heavy chain CDRs. Gray squares represent amino acid substitutions that reduce binding, compared to the original sequences of L1 CDR residues at the same positions. The darker the gray shade, the weaker the binding compared to the original residues.

FIG. 30 shows the heatmap of positive selection of the mutants within the L1 heavy chain CDRs. Gray squares represent amino acid substitutions that enhance/increase binding, compared to the original sequences of L1 CDR residues at the same positions. The darker the gray shade, the stronger the binding compared to the original residues.

FIG. 31 shows the heatmap of negative selection of the mutants within the L1 light chain CDRs.

FIG. 32 shows the heatmap of positive selection of the mutants within the L1 light chain CDRs.

FIG. 33 shows the heatmap of negative selection of the mutants within the L2 heavy chain CDRs.

FIG. 34 shows the heatmap of positive selection of the mutants within the L2 heavy chain CDRs.

FIG. 35 shows the heatmap of negative selection of the mutants within the L2 light chain CDRs.

FIG. 36 shows the heatmap of positive selection of the mutants within the L2 light chain CDRs.

FIGS. 37A-37D shows binding affinities of representative L1 and L2 antibodies of the invention for the human (FIG. 37A), monkey (FIG. 37B) and mouse (FIG. 37C) IGSF8 expressed on the surface of CT26 cells, and EC50 values thereof measured by FACS (FIG. 37D).

FIG. 38A shows knock-down of KIR3DL2 by lentiviral-mediated CRISPR/Cas9 on NK cells as measured by FACS. FIG. 38B shows that IGSF8-mediated suppression of K562 cell killing by human primary NK cells can be reversed by loss of KIR3DL2 on the NK cells.

FIG. 39 shows by FACS that the representative L1 and L2 antibodies can fully block the interaction of IGSF8 and KIR3DL2 in a dose dependent manner.

FIGS. 40A-40D shows in vitro anti-tumor cell efficacy of the representative L1 and L2 antibodies using the co-culture model of primary NK cell and cancer cell lines Jurkat (FIG. 40A), SU-DHL2 (FIG. 40B), LNCap (FIG. 40C) and K562 (FIG. 40D). ****: P<0.0001.

FIGS. 41A-41B shows in vitro anti-tumor cell efficacy of the representative L1 and L2 antibodies using the co-culture model of PBMC and cancer cell lines H1437 (FIG. 41A) and SKBR3 (FIG. 41B). ****: P<0.0001.

FIGS. 42A-42B shows in vitro anti-tumor cell efficacy of the representative L1 and L2 antibodies using the co-culture model of PBMC and cancer cell lines SW480 (FIG. 42A) and H520 (FIG. 42B). Efficacy of L1 or L2 antibodies with normal human IgG1 or deficient mutant of IgG1 (IgG1-LALA) were compared. **: P<0.01; ***: P<0.001; ****: P<0.0001.

FIG. 43A shows the in vivo anti-tumor efficacy of the representative L1 antibodies using the B16-F10 syngeneic model. FIG. 43B depicts comparison between the L1 antibodies with normal human IgG1, IgG4, and the deficient mutant of IgG1 (IgG1-LALA). **: P<0.01; ***: P<0.001; ****: P<0.0001.

FIG. 44 shows expression of marker gene of effector NK and T cells in the B16 tumors treated by L1 antibodies with human normal IgG1, IgG4, and the deficient mutant of IgG1 (IgG1-LALA). *: P<0.05**: P<0.01.

### DETAILED DESCRIPTION OF THE INVENTION

### 1. Overview

The Immunoglobulin Superfamily Member 8 (IGSF8) gene encodes a member of the immunoglobulin protein superfamily, with a single transmembrane (TM) domain. IGSF8 contains an extracellular Ig V-set domain, which is found in diverse protein families, including T-cell receptors such as CD2, CD4, CD80 and CD86, as well as immune checkpoints such as PD1, LAGS, PDL1. In human, IGSF8 appears to be over-expressed in histologic tissues from selected cancer patients when compared to control levels in normal human tissues.

The invention described herein is partly based on the discovery that IGSF8 is a novel cancer treatment target, and thus antagonists of IGSF8 can be used to treat such cancer. The data presented herein demonstrate that IGSF8 is uniquely expressed in cancer cells, and is highly expressed in multiple cancer types, particularly in melanoma, cervical cancer, non-small cell lung cancer, colorectal cancer, and a number of other cancers. IGSF8 interacts with T and NK (natural killer) cells to prevent NK and T cell proliferation and/or reduces the viability of NK and T cells. Meanwhile, knocking out IGSF8 gene or otherwise inactivating IGSF8 function improves tumor infiltration by T and NK cells, and enhances their cytolytic activities in vivo.

More specifically, the present invention is partly based on the discovery that IGSF8 has a previously unrecognized function as a novel inhibitory ligand for activated NK cells, and serves as an immune checkpoint to regulate NK cell-mediated immune surveillance of cancer. IGSF8 recombinant protein suppresses proliferation and cytolytic activity of activated primary NK or T cells. On the other hand, IGSF8 inhibition (such as by anti-IGSF8 monoclonal antibodies) leads to in vivo efficacy in multiple rodent oncologic animal models.

Invention described herein, which is partly based on inhibiting IGSF8-mediated NK cell function, is advantageous over MHC class I (HLA)-based NK cell inhibition, partly due to the fact that MHC I molecules are highly diverse among unrelated individuals, while IGSF8 is not only non-polymorphic among different individuals, but also conserved to a large extent across species (such as conserved to a high degree between human and experimental animals like mouse), thus enabling the testing of anti-IGSF8 agents, including anti-human IGSF8 monoclonal antibodies directly in animal (e.g., mouse) models.

The invention described herein is further based on the discovery that IGSF8 can specifically bind to primary NK cells through its D1 domain—an Ig V-set domain, as a truncated IGSF8 having only the D1 domain as the extracellular domain is sufficient for NK cell suppression, while another truncated IGSF8 protein without only the D1 domain completely loses the suppressive functions for NK cells.

The invention described herein is further based on the discovery that IGSF8 binds to NK cells through specifically binding to a KIR family receptor—KIR3DL2 (and to a lesser extent, KIR3DL1)—that is expressed on the surface of NK cells. Just like tumors can escape T cell-mediated immunity by down-regulating MHC-I or expressing PD-L1 ligand to inhibit T cell function by binding to PD1 on T cells, tumors can similarly up-regulate IGSF8 to evade NK cell-mediated immune surveillance of cancer by binding to the KIR receptors specific for IGSF8 (e.g., KIR3DL1/2) on NK cells.

The invention described herein is further based on the discovery that IGSF8 binds to NK cells through specifically binding to a KLRC1/KLRD1 heterodimeric receptor (but not KLRC1 or KLRD1 monomer alone) that is expressed on the surface of NK cells. As discussed above, tumors can up-regulate IGSF8 to evade NK cell-mediated immune surveillance of cancer by binding to the KLRC1/D1 heterodimeric receptors specific for IGSF8 on NK cells.

As IGSF8 has been found to express at high levels in multiple types of tumors, immune therapies using anti-IGSF8 mAbs as checkpoint inhibitors can increase the pool of patients that respond to checkpoint inhibitor treatment. Furthermore, patients with tumors that have developed resistance to PD-1 therapy may also express IGSF8 as an alternative immune evasion strategy, and IGSF8 blockade may offer an additional avenue to overcome resistance to PD-1 immunotherapy.

The invention described herein is further based on the discovery that anti-IGSF8 therapy works synergistically with anti-PD1/PD-L1 therapy, partly by activating both T and NK cells in the tumor microenvironment, as demonstrated by animal models herein.

Accordingly, the present invention provides monoclonal antibodies, and antigen binding fragments thereof, that specifically bind to IGSF8 (particularly to its Ig V-set extracellular domain). Such antibodies may inhibit one or more functions of IGSF8, such as IGSF8 binding to an NK cell surface receptor (e.g., KIR3DL1 or KIR3DL2 or KLRC1/D1), and reverses or reduces IGSF8-mediated inhibition of NK cell activity and/or viability. The present invention further provides nucleic acids encoding the anti-IGSF8 antibodies or antigen-binding fragments thereof, vectors carrying such nucleic acid coding sequences for expression in a suitable host cell, as well as methods of producing such antibodies or antigen-binding fragments thereof by culturing host cells capable of expressing such antibodies or antigen-binding fragments thereof. The present invention further provides methods of using such antibodies for diagnostic, prognostic, and therapeutic purposes.

Multiple antibodies have been generated against IGSF8, many of which have been validated for IGSF8 binding, blocking, exhibiting ADCC towards cancer cells expressing IGSF8, and enhancement of cancer cell killing by NK and/or T cells. More importantly, the data presented herein showed that simultaneously inhibiting IGSF8 function and the PD-1/PD-L1 immune checkpoint led to synergistic efficacy in an in vivo mouse model of cancer (melanoma).

The antibodies described herein are partly characterized by the high binding affinity of the antibodies against IGSF8. The antibodies described herein are further partly based on the surprising discovery that certain formats of antibodies with reduced effector function exhibit better anti-tumor efficacy than antibodies with full effector function.

The present invention also provides monoclonal antibodies, and antigen binding fragments thereof, that specifically bind to one of the IGSF8 receptors on NK cells and/or on T cells, such as KIR3DL1 or KIR3DL2 or KLRC1/D1, to reverse or reduce IGSF8-mediated inhibition of NK/T cell activity and/or viability by IGSF8 binding to one or more of these receptors. Antibodies specific for KIR3DL2 or KIR3DL1 may be specific for the D2 extracellular domain of KIR3DL1/2 responsible for IGSF8 binding, including antibodies that specifically blocks IGSF8 binding to residues S165, I171, and/or M186 of KIR3DL1/2. Such antibodies may inhibit one or more functions of KIR3DL1/2 and/or KLRC1/D1, such as IGSF8 binding, and reverses or reduces IGSF8-mediated inhibition of NK cell activity and/or viability. The present invention further provides nucleic acids encoding such antibodies or antigen-binding fragments thereof directed towards KIR3DL1 or KIR3DL2 or KLRC1/D1, vectors carrying such nucleic acid coding sequences for expression in a suitable host cell, as well as methods of producing such antibodies or antigen-binding fragments thereof by culturing host cells capable of expressing such antibodies or antigen-binding fragments thereof. The present invention further provides methods of using such antibodies for diagnostic, prognostic, and therapeutic purposes.

Thus the invention described herein specifically provides methods and reagents for modulating an immune response, or for treating cancer, by modulating (e.g., inhibiting) IGSF8 activity/antagonizing IGSF8 function, by disrupting/antagonizing its interaction with one or more of its receptors on NK/T cells (e.g., KIR3DL1 or KIR3DL2 or KLRC1/D1), with optional combination with an optional second therapeutic agent targeting the PD-1/PD-L1 immune checkpoint.

Detailed aspects of the invention are described further and separately in the various sections below. However, it should be understood that any one embodiment of the invention, including embodiments described only in the examples or drawings, and embodiments described only under one section below, can be combined with any other embodiment(s) of the invention.

### 2. Definitions

The term “antibody,” in the broadest sense, encompasses various antibody structures, including but not limited to monoclonal antibodies, polyclonal antibodies, and multispecific antibodies (e.g., bispecific antibodies). The term “antibody” may also broadly refers to a molecule comprising complementarity determining region (CDR) 1, CDR2, and CDR3 of a heavy chain and CDR1, CDR2, and CDR3 of a light chain, wherein the molecule is capable of binding to an antigen. The term “antibody” also includes, but is not limited to, chimeric antibodies, humanized antibodies, human antibodies, and antibodies of various species such as mouse, human, cynomolgus monkey, etc.

In a narrower sense, however, “antibody” refers to the various monoclonal antibodies, including chimeric monoclonal antibodies, humanized monoclonal antibodies, and human monoclonal antibodies.

In some embodiments, an antibody comprises a heavy chain variable region (HCVR or VH) and a light chain variable region (LCVR or VL). In some embodiments, an antibody comprises at least one heavy chain (HC) comprising a heavy chain variable region and at least a portion of a heavy chain constant region, and at least one light chain (LC) comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region.

As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region (HCVR or VH)” as used herein refers to, at a minimum, a heavy chain CDR1 (CDR-H1 or VH-CDR1), framework 2 (HFR2 or VH-FR2), CDR2 (CDR-H2 or VH-CDR2), FR3 (HFR3 or VH-FR3), and CDR3 (CDR-H3 or VH-CDR3). In some embodiments, a heavy chain variable region also comprises at least a portion of an FR1 (HFR1 or VH-FR1), which is N-terminal to CDR-H1, and/or at least a portion of an FR4 (HFR4 or VH-FR4), which is C-terminal to CDR-H3.

The term “heavy chain constant region” as used herein refers to a region comprising at least three heavy chain constant domains, CH1, CH2, and CH3. Non-limiting exemplary heavy chain constant regions include γ, δ, and α. Non-limiting exemplary heavy chain constant regions also include c and Each heavy constant region corresponds to an antibody isotype. For example, an antibody comprising a γ constant region is an IgG antibody (e.g., IgG1, IgG2, IgG3, IgG4), an antibody comprising a δ constant region is an IgD antibody, an antibody comprising an a constant region is an IgA antibody, an antibody comprising an ε constant region is an IgE antibody, and an antibody comprising an μ constant region is an IgM antibody.

Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an α1 constant region) and IgA2 (comprising an α2 constant region) antibodies; and IgM antibodies include, but are not limited to, IgM1 (comprising an μl constant region) and IgM2 (comprising an μ2 constant region).

The heavy chain constant region contains a fragment crystalizatble (Fc) domain at the C-end of the molecule. A major function of the Fc region is to evoke immune effector function, such as antibody-dependent cellular cytotoxicity (ADCC), complement-dependent cytotoxicity (CDC) and antibody-dependent cellular phagocytosis (ADCP), through interactions with cell surface receptors called Fc receptors (FcR) and some protein of the complement system (e.g. C1q). Different antibody isotypes may engage immune effector function at varying degrees, and Fc engineering strategies have also been employed to enhance or reduce immune effector function.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence, and with or without a C-terminal lysine.

The term “light chain variable region (LCVR or VL)” as used herein refers to a region comprising light chain CDR1 (CDR-L1 or VL-CDR1), framework (FR) 2 (LFR2 or VL-FR2), CDR2 (CDR-L2 or VL-CDR2), FR3 (LFR3 or VL-FR3), and CDR3 (CDR-L3 or VL-CDR3). In some embodiments, a light chain variable region also comprises at least a portion of an FR1 (LFR1 or VL-FR1) and/or at least a portion of an FR4 (LFR4 or VL-FR4).

The term “light chain constant region” as used herein refers to a region comprising a light chain constant domain, C_{L}. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The term “antibody fragment” or “antigen binding portion” (of antibody) includes, but is not limited to, fragments that are capable of binding antigen, such as Fv, single-chain Fv (scFv), Fab, Fab′, and (Fab′)₂.

An “antibody that binds to the same epitope” as a reference antibody can be determined by an antibody competition assay. It refers to an antibody that blocks binding of the reference antibody to its antigen in a competition assay by 50% or more, and conversely, the reference antibody blocks binding of the antibody to its antigen in a competition assay by 50% or more. The term “compete” when used in the context of an antibody that compete for the same epitope means competition between antibodies is determined by an assay in which an antibody being tested prevents or inhibits specific binding of a reference antibody to a common antigen.

Numerous types of competitive binding assays can be used, for example: solid phase direct or indirect radioimmunoassay (RIA), solid phase direct or indirect enzyme immunoassay (ETA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using I¹²⁵ label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol.).

Typically, such an assay involves the use of purified antigen bound to a solid surface or cells bearing either of these, an unlabeled test antigen binding protein and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibodies and antibodies binding to an adjacent epitope sufficiently proximal to the epitope bound by the reference antibody for steric hindrance to occur. In some embodiments, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antibody or immunologically functional fragment thereof, and additionally capable of being used in a mammal to produce antibodies capable of binding to that antigen. An antigen may possess one or more epitopes that are capable of interacting with antibodies.

The term “epitope” is the portion of an antigen molecule that is bound by a selective binding agent, such as an antibody or a fragment thereof. The term includes any determinant capable of specifically binding to an antibody. An epitope can be contiguous or non-contiguous (e.g., in a polypeptide, amino acid residues that are not contiguous to one another in the polypeptide sequence but that within in context of the molecule are bound by the antigen binding protein). In some embodiments, epitopes may be mimetic in that they comprise a three dimensional structure that is similar to an epitope used to generate the antibody, yet comprise none or only some of the amino acid residues found in that epitope used to generate the antibody. Epitope determinants may include chemically active surface groupings of molecules such as amino acids, sugar side chains, phosphoryl or sulfonyl groups, and may have specific three dimensional structural characteristics, and/or specific charge characteristics.

In some embodiments, an “epitope” is defined by the method used to determine it. For example, in some embodiments, an antibody binds to the same epitope as a reference antibody, if they bind to the same region of the antigen, as determined by hydrogen-deuterium exchange (HDX).

In certain embodiments, an antibody binds to the same epitope as a reference antibody if they bind to the same region of the antigen, as determined by X-ray crystallography.

A “chimeric antibody” as used herein refers to an antibody comprising at least one variable region from a first species (such as mouse, rat, cynomolgus monkey, etc.) and at least one constant region from a second species (such as human, cynomolgus monkey, chicken, etc.). In some embodiments, a chimeric antibody comprises at least one mouse variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof. In some embodiments, a humanized antibody fragment is an Fab, an scFv, a (Fab′)₂, etc.

A “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequences.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Non-limiting exemplary mammalian cells include, but are not limited to, NSO cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a mammal such as human. In some embodiments, methods of treating other non-human mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a (human) subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” or “reference tissue,” as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

A “disorder” or “disease” is any condition that would benefit from treatment with one or more IGSF8 antagonists of the invention. This includes chronic and acute disorders or diseases including those pathological conditions that predispose the mammal to the disorder in question. Non-limiting examples of disorders to be treated herein include cancers.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells (i.e., forming solid tumors) or leukemic cancer cells. The term “cancer growth” is used herein to refer to proliferation or growth by a cell or cells that comprise a cancer that leads to a corresponding increase in the size or extent of the cancer.

A “chemotherapeutic agent” is a chemical compound that can be useful in the treatment of cancer. Examples of chemotherapeutic agents include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl, 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Illinois), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above.

Further non-limiting exemplary chemotherapeutic agents include anti-hormonal agents that act to regulate or inhibit hormone action on cancers such as anti-estrogens and selective estrogen receptor modulators (SERMs), including, for example, tamoxifen (including NOLVADEX® tamoxifen), raloxifene, droloxifene, 4-hydroxy tamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (AVASTIN®) or to the VEGF-A receptor (e.g., KDR receptor or Flt-1 receptor), anti-PDGFR inhibitors such as GLEEVEC® (Imatinib Mesylate), small molecules that block VEGF receptor signaling (e.g., PTK787/ZK2284, SU6668, SUTENT®/SUl 1248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D′Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12): 1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents (also referred to as immuno-oncology agents), apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (TARCEVA®), platelet derived growth factor inhibitors (e.g., GLEEVEC® (Imatinib Mesylate)), a COX-2 inhibitor (e.g., celecoxib), interferons, CTLA4 inhibitors (e.g., anti-CTLA antibody ipilimumab (YERVOY®)), PD-1 inhibitors (e.g., anti-PD1 antibodies, BMS-936558), PDL1 inhibitors (e.g., anti-PDL1 antibodies, MPDL3280A), PDL2 inhibitors (e.g., anti-PDL2 antibodies), VISTA inhibitors (e.g., anti-VISTA antibodies), cytokines, antagonists (e.g., neutralizing antibodies) that bind to one or more of the following targets ErbB2, ErbB3, ErbB4, PDGFR-beta, BlyS, APRIL, BCMA, PD-1, PDL1, PDL2, CTLA4, VISTA, or VEGF receptor(s), TRAIL/Apo2, and other bioactive and organic chemical agents, etc. Combinations thereof are also included in the invention.

“Treatment” refers to therapeutic treatment, for example, wherein the object is to slow down (lessen) the targeted pathologic condition or disorder as well as, for example, wherein the object is to inhibit recurrence of the condition or disorder. “Treatment” covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of IGSF8 antagonist of the invention may vary according to factors such as the disease state, age, sex, and weight of the individual, and the ability of the antagonist to elicit a desired response in the individual. A therapeutically effective amount encompasses an amount in which any toxic or detrimental effects of IGSF8 antagonist are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

An “article of manufacture” is any manufacture (e.g., a package or container) or kit comprising at least one reagent, e.g., a medicament for treatment of a disease or disorder, or a probe for specifically detecting a biomarker described herein. In some embodiments, the manufacture or kit is promoted, distributed, or sold as a unit for performing the methods described herein.

### 3. Methods of Treating Cancer

The invention described herein provides modulators, e.g., antagonists for IGSF8 (e.g., isolated or recombinant monoclonal antibodies or an antigen-binding fragments thereof specific for IGSF8) and its receptors (such as KIR3DL1/2, KLRC1/D1) for use in methods of treating humans and other non-human mammals, such as an animal model of a cancer.

In one aspect, the invention provides a method for modulating an immune response in a subject in need thereof, the method comprising inhibiting interaction between IGSF8 and a receptor of IGSF8 selected from KIR3DL1, KIR3DL2, and KLRC1/D2 heterodimer. In certain embodiments, the method comprises administrating the anti-IGSF8 monoclonal antibody or antigen-binding fragment thereof (such as those described herein) of the invention to the subject.

In another aspect, the invention provides a method of immunotherapy for treating a cancer in a subject in need thereof, the method comprising inhibiting interaction between IGSF8 and a receptor of IGSF8 selected from KIR3DL1, KIR3DL2, and KLRC1/D2 heterodimer. In certain embodiments, the method comprises administrating the anti-IGSF8 monoclonal antibody or antigen-binding fragment thereof (such as those described herein) of the invention to the subject.

In yet another aspect, the invention provides a method for treating or preventing a cancer in a subject in need thereof, the method comprising administering to the subject in need of such treatment a therapeutically effective amount of an IGSF8, KIR3DL1/2, or KLRC1/D1 modulator (e.g., antagonists, such as antibodies or antigen-binding portion/fragment) of the invention.

Specifically, the invention provides a method of treating cancer in a subject in need thereof, the method comprising administering to the subject a therapeutically effective amount of an IGSF8 (Immuno Globulin Super Family 8) modulator (e.g., antagonist).

The invention also provides a method of treating cancer in a subject in need thereof, the method comprising administering to the subject a therapeutically effective amount of an KIR3DL1 antagonist that inhibits interaction with IGSF8.

The invention further provides a method of treating cancer in a subject in need thereof, the method comprising administering to the subject a therapeutically effective amount of an KIR3DL2 antagonist that inhibits interaction with IGSF8.

The invention additional provides a method of treating cancer in a subject in need thereof, the method comprising administering to the subject a therapeutically effective amount of an KLRC1/D1 antagonist that inhibits interaction with IGSF8.

In some embodiments, methods of treating cancer are provided, wherein the methods comprise administering an effective amount of IGSF8, KIR3DL1/2, or KLRC1/D1 modulator (e.g., antagonists, such as antibodies or antigen-binding portion/fragment) of the invention, to a subject with cancer in need of treatment.

In some embodiments, use of an effective amount of IGSF8, KIR3DL1/2, or KLRC1/D1 modulator (e.g., antagonists, such as antibodies or antigen-binding portion/fragment) of the invention, for treating cancer is provided.

Non-limiting exemplary cancers that may be treated with IGSF8 antagonists (e.g., an anti-IGSF8 monoclonal antibody or antigen-binding fragment thereof of the invention) are provided herein, including carcinoma, lymphoma, blastoma, sarcoma, and leukemia. More particular non-limiting examples of such cancers include melanoma, cervical cancer, squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulvar cancer, thyroid cancer, hepatic carcinoma, brain cancer, endometrial cancer, testis cancer, cholangiocarcinoma, gallbladder carcinoma, gastric cancer, melanoma, and various types of head and neck cancer.

In certain embodiment, cancers treatable with the method of the invention, using the IGSF8, KIR3DL1/2, or KLRC1/D1 modulator (e.g., antagonists, such as antibodies or antigen-binding portion/fragment) of the invention, include but are not limited to: carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulvar cancer, thyroid cancer, hepatic carcinoma, brain cancer, endometrial cancer, testis cancer, cholangiocarcinoma, gallbladder carcinoma, gastric cancer, melanoma, and various types of head and neck cancer.

Additional treatable cancers include melanoma (including skin cutaneous melanoma), cervical cancer, lung cancer (e.g., non-small cell lung cancer, lung adenocarcinoma, lung squamous cell carcinoma), colorectal cancer, lymphoma (including B cell lymphoma and DLBCL), leukemia (including CLL and Acute Myeloid Leukemia (AML)), BLCA tumor, breast cancer, head and neck carcinoma, head-neck squamous cell carcinoma, PRAD, THCA, or UCEC, thyroid cancer, unitary tract cancer, uterine cancer, esophagus cancer, liver cancer, ganglia cancer, renal cancer, pancreatic cancer, pancreatic ductal carcinoma, ovarian cancer, prostate cancer, gliomas, glioblastoma, neuroblastoma, thymoma, B-CLL, and a cancer infiltrated with immune cells expressing a receptor to IGSF8.

In certain embodiments, the treatable cancer is lung cancer, renal cancer, pancreatic cancer, colorectal cancer, acute myeloid leukemia (AML), head and neck carcinoma, liver cancer, ovarian cancer, prostate cancer, or uterine cancer.

In some embodiments, the lung cancer is non-small cell lung cancer or lung squamous cell carcinoma.

In some embodiments, the leukemia is acute myeloid leukemia (AML) or chronic lymphocytic leukemia (CLL).

In some embodiments, the breast cancer is breast invasive carcinoma.

In some embodiments, the ovarian cancer is ovarian serous cystadenocarcinoma.

In some embodiments, the kidney cancer is kidney renal clear cell carcinoma.

In some embodiments, the colon cancer is colon adenocarcinoma.

In some embodiments, the bladder cancer is bladder urothelial carcinoma.

In some embodiments, the cancer cells and/or tumor immune infiltrating cells in the subject express IGSF8.

While not wishing to be bound by any particular theory, the methods of the invention may be based on at least partial relief of IGSF8-mediated inhibition of host innate and/or adapted immune system, exerted on the effector cells of the host innate/adapted immune system, such as NK cells and/or (CD8⁺) T cells. Such inhibition may be effected by engaging one or more IGSF8 receptors (such as KIR3DL1/2 nad KLRC1/D1) upon IGSF8 binding, and such inhibition may be at least partially relieved by interrupting IGSF8 binding to these receptors expressed on the effectors of the host innate/adaptive immune system (e.g., NK cells or T cells). Thus the method of the invention may not rely on (but do not necessarily exclude) the conventional ADCC- or CDC-mediated killing of target cells by innate immune system cells (e.g., NK cells) based on antibodies on the surface of these target cells overexpressing one of the IGSF8 receptors (such as KIR3DL1/2 nad KLRC1/D1).

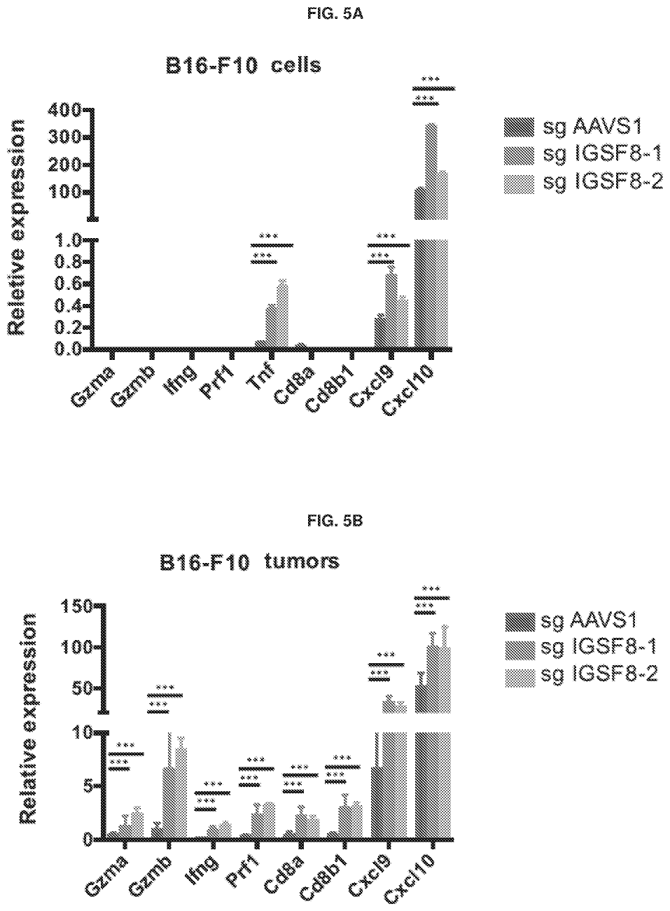

Thus, in some embodiments, the cancer is treatable by inhibiting binding between IGSF8 and at least one of its receptors, such as KIR3DL1/2 and KLRC1/D1. In some embodiments, the cancer expresses IGSF8. See, for example, any cancer described in FIG. 6A, 6B or 6C with IGSF8 expression.

In some embodiments, the cancer is not characterized by expression or overexpression of KIR3DL1/2. In some embodiments, the cancer is not cutaneous T-cell lymphomas, such as Sézary syndrome, CD30⁺ cutaneous lymphoma, and transformed mycosis fungoides.

In some embodiments, the cancer is not characterized by expression or overexpression of KLRC1/D1.

In some embodiments, the KIR3DL1 antagonist is selected from an anti-KIR3DL1 antibody or an antigen-binding portion/fragment thereof, an inhibitory peptide of KIR3DL1, a nucleic acid targeting KIR3DL1 (an aptamer, an antisense polynucleotide, an RNAi reagent such as siRNA, miRNA, shRNA; a guide RNA for a Type 2 CRISPR/Cas effector enzyme), or a small molecule targeting KIR3DL1 (e.g., with M.W.<1000 Da or <500 Da); optionally, the KIR3DL1 antagonist is the anti-KIR3DL1 antibody or antigen-binding portion/fragment thereof.

In some embodiments, the KIR3DL2 antagonist is selected from an anti-KIR3DL2 antibody or an antigen-binding portion/fragment thereof, an inhibitory peptide of KIR3DL2, a nucleic acid targeting KIR3DL2 (an aptamer, an antisense polynucleotide, an RNAi reagent such as siRNA, miRNA, shRNA; a guide RNA for a Type 2 CRISPR/Cas effector enzyme), or a small molecule targeting KIR3DL2 (e.g., with M.W.<1000 Da or <500 Da); optionally, the KIR3DL2 antagonist is the anti-KIR3DL2 antibody or antigen-binding portion/fragment thereof.

In some embodiments, the anti-KIR3DL1/2 antibody or antigen-binding portion/fragment thereof, the inhibitory peptide against KIR3DL1/2, the nucleic acid targeting KIR3DL1/2, or the small molecule targeting KIR3DL1/2 binds to an epitope of KIR3DL1/2 comprising residue S165, I171, and/or M186, thereby inhibiting IGSF8 binding to the D2 domain of KIR3DL1/2.

In some embodiments, the anti-KIR3DL1/2 antibody or antigen-binding portion/fragment thereof specifically binds the middle/D2 Ig-like domain of the ECD of KIR3DL1/2, optionally, the anti-KIR3DL1/2 antibody or antigen-binding portion/fragment thereof specifically binds an epitope comprising residues S165, I171, and/or M186.

In some embodiments, the KLRC1/D1 antagonist is selected from an anti-KLRC1/D1 antibody or an antigen-binding portion/fragment thereof, an inhibitory peptide of KLRC1/D1, a nucleic acid targeting KLRC1/D1 (an aptamer, an antisense polynucleotide, an RNAi reagent such as siRNA, miRNA, shRNA; a guide RNA for a Type 2 CRISPR/Cas effector enzyme), or a small molecule targeting KLRC1/D1 (e.g., with M.W.<1000 Da or <500 Da); optionally, the KLRC1/D1 antagonist is the anti-KLRC1/D1 antibody or antigen-binding portion/fragment thereof.

In some embodiments, the IGSF8 antagonist is an anti-IGSF8 antibody or an antigen-binding portion/fragment thereof, an inhibitory peptide of IGSF8, a nucleic acid targeting IGSF8 (an aptamer, an antisense polynucleotide, an RNAi reagent such as siRNA, miRNA, shRNA; a guide RNA for a Type 2 CRISPR/Cas effector enzyme), or a small molecule targeting IGSF8 (e.g., with M.W.<1000 Da or <500 Da); optionally, the IGSF8 antagonist is the anti-IGSF8 antibody or antigen-binding portion/fragment thereof.

In some embodiments, the IGSF8 antagonist is selected from an anti-IGSF8 antibody or an antigen-binding fragment thereof. In some embodiments, the antibody is a chimeric antibody, a humanized antibody, or a human antibody. In some embodiments, the anti-IGSF8 antibody or antigen-binding fragment thereof binds to the terminal Ig-V set ECD or D1 of IGSF8. In some embodiments, the anti-IGSF8 antibody or antigen-binding fragment thereof inhibits IGSF8 binding to KIR3DL1/2, such as the middle/D2 domain of KIR3DL1 and/or KIR3DL2, e.g., an epitope comprising residue S165, I171, and/or M186 of KIR3DL1/2.

In some embodiments, the antigen-binding portion/fragment is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In some embodiments, the anti-IGSF8 antibody or antigen-binding portion/fragment thereof is any one of the monoclonal antibody, or antigen-binding portion/fragment thereof described herein (see section for IGSF8 antagonist, e.g., anti-IGSF8 antibodies).

In some embodiments, the IGSF8 antagonist (e.g., an anti-IGSF8 monoclonal antibody or antigen-binding fragment thereof of the invention) promotes expression, secretion, or otherwise increases activity of a cytokine or a target gene selected from the group consisting of: CXCL10, CXCL9, TNFα, CD8b, CD8a, Prfl, IFNγ, Gzma, Gzmb, CD274, PDCD1, PDCD1 Ig2, LAG3, Havcr2, Tigit, or CTLA4.

In some embodiments, expression, secretion, or otherwise increased activity of said cytokine or said target gene occurs within tumor microenvironment.

In some embodiments, expression, secretion, or otherwise increased activity of said cytokine or said target gene is due to immune cell (e.g., T lymphocytes or NK cells) infiltration into tumor microenvironment.

In some embodiments, the anti-IGSF8 and/or anti-KIR3DL1/2 and/or anti-KLRC1/D1 antibody or antigen-binding portion/fragment thereof is conjugated to a cytotoxic agent. The cytotoxic agent can be selected from the group consisting of a chemotherapeutic agent, a biologic agent, a toxin, and a radioactive isotope.

In some embodiments, the IGSF8 antagonist, the KIR3DL1 antagonist, the KIR3DL2 antagonist, or the KLRC1/D1 antagonist is an immunostimulatory molecule.

In some embodiments, the IGSF8 antagonist (e.g., an anti-IGSF8 monoclonal antibody or antigen-binding fragment thereof of the invention), the KIR3DL1 antagonist, the KIR3DL2 antagonist, or the KLRC1/D1 antagonist stimulates T cell or NK cell activation and/or infiltration into tumor microenvironment.

In some embodiments, the anti-IGSF8 and/or anti-KIR3DL1/2 and/or the anti-KLRC1/D1 antibody or antigen-binding portion/fragment thereof reduces the number of proliferating cells in the cancer and/or reduces the volume or size of a tumor of the cancer.

In some embodiments, the anti-IGSF8 and/or anti-KIR3DL1/2 and/or the anti-KLRC1/D1 antibody or antigen-binding portion/fragment thereof is administered in a pharmaceutically acceptable formulation.

In some embodiments, the anti-IGSF8 antibody or antigen-binding fragment thereof (e.g., F(ab′)₂ fragment) is administered with a second therapeutic agent (see combination therapy section, incorporated herein by reference).

In some embodiments, the anti-IGSF8, the anti-KIR3DL1/2, or the anti-KLRC1/D1 antibody or antigen-binding fragment thereof is administered with a second immune checkpoint inhibitor, such as an immune checkpoint inhibitor that restores or promotes T-cell mediated immunotherapy.

In some embodiments, the immune checkpoint inhibitor is an antibody or antigen-binding fragment thereof specific for PD-1, PD-L1, PD-L2, LAG3, TIGIT, TIM3, NKG2A, CD276, VTCN1, VISR or HHLA2.

In some embodiments, the anti-IGSF8, the anti-KIR3DL1/2, or the anti-KLRC1/D1 antibody or antigen-binding fragment thereof is administered with an anti-PD-1 antibody or antigen-binding fragment thereof, an anti-PD-L1 antibody or antigen-binding fragment thereof, and/or an anti-CTLA-4 antibody or antigen-binding fragment thereof. In some embodiments, the anti-IGSF8 antibody is a human antibody.

In some embodiments, the immune checkpoint inhibitor is an anti-PD-1 antibody, such as cemiplimab, nivolumab, or pembrolizumab.

In some embodiments, the immune checkpoint inhibitor is an anti-PD-L1 antibody, such as avelumab, durvalumab, atezolizumab, KN035, or CK-301.

In some embodiments, the immune checkpoint inhibitor is a (non-antibody) peptide inhibitor of PD-1/PD-L1, such as AUNP12; a small molecule inhibitor of PD-L1 such as CA-170, or a macrocyclic peptide such as BMS-986189.

In certain embodiments, the combination therapy further includes a therapeutic antibody effective to treat the cancer or immunological condition. Exemplary therapeutic antibodies include: 3F8, 8H9, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Amivantamab, Anatumomab mafenatox, Andecaliximab, Anetumab ravtansine, Anifrolumab, Anrukinzumab, Apolizumab, Aprutumab ixadotin, Arcitumomab, Ascrinvacumab, Aselizumab, Atezolizumab, Atidortoxumab, Atinumab, Atorolimumab, Avelumab, Azintuxizumab vedotin, Bapineuzumab, Basiliximab, Bavituximab, BCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, BirtamimabBivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab, Carlumab, Carotuximab, Catumaxomab, cBR-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, GancotamabGanitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ianalumab, Ibalizumab, IBI308, Ibritumomab tiuxetan, Icrucumab, Idarucizumab, Ifabotuzumab, Igovomab, Iladatuzumab vedotin, IMAB363, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Mapatumumab, Margetuximab, MarstacimabMaslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, OtilimabOtlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, RanevetmabRanibizumab, Raxibacumab, Ravagalimab, Ravulizumab, Refanezumab, Regavirumab, REGN-EB, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Talquetamab, Tamtuvetmab, Tanezumab, Taplitumomab paptox, Tarextumab, TavolimabTeclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, tiragolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab, Zolbetuximab, (=IMAB362, Claudiximab), Zolimomab aritox, or combination thereof.

In certain embodiments, the second therapeutic agent comprises an antibody or an antigen-binding portion/fragment thereof effective to induce ADCC, ADCP, and/or CDC.

In some embodiments, the IGSF8 antagonist for treating cancer may be a non-antibody protein, such as a soluble version of the IGSF8 protein or a portion thereof (e.g., the Ig-V set ECD) that inhibits the interaction between IGSF8 and its ligand, optionally further comprising a fusion partner and in the form of a fusion molecule, such as (IgG1) Fc fusion. Various exemplary IGSF8 antagonists are described in more detail in the sections that follow.

In some embodiments, the KIR3DL1/2 antagonist for treating cancer may be a non-antibody protein, such as a soluble version of the KIR3DL1/2 protein or a portion thereof (e.g., the 2^{nd} Ig domain of the ECD) that inhibits the interaction between IGSF8 and KIR3DL1/2, optionally further comprising a fusion partner and in the form of a fusion molecule, such as (IgG1) Fc fusion.

In some embodiments, the KLRC1/D1 antagonist for treating cancer may be a non-antibody protein, such as a soluble version of the KLRC1/D1 protein or a portion thereof (e.g., the ECD) that inhibits the interaction between IGSF8 and KLRC1/D1, optionally further comprising a fusion partner and in the form of a fusion molecule, such as (IgG1) Fc fusion.

The invention described herein also provides KIR3DL1/2 or KLRC1/D1 antagonists for use in methods of treating humans and other non-human mammals.

In some embodiments, methods for treating or preventing a cancer are provided, comprising administering an effective amount of KIR3DL1/2 or KLRC1/D1 antagonist to a subject in need of such treatment.

In some embodiments, methods for activating NK cell, such as activating NK cell-mediated immunotherapy (which can be useful for treating or preventing a cancer) are provided, comprising contacting NK cells with KIR3DL1/2 or KLRC1/D1 antagonist, or administering an effective amount of KIR3DL1/2 or KLRC1/D1 antagonist to a subject in need of such NK cell-mediated immunotherapy.

In some embodiments, methods of treating cancer are provided, wherein the methods comprise administering KIR3DL1/2 or KLRC1/D1 antagonist to a subject with cancer.

In some embodiments, use of KIR3DL1/2 or KLRC1/D1 antagonist for treating cancer is provided.

In some embodiments, the cancer is treatable by inhibiting binding between IGSF8 and KIR3DL1/2 and/or KLRC1/D1. In some embodiments, the cancer expresses IGSF8. In some embodiments, the cancer is not characterized by expression or overexpression of KIR3DL1/2. In some embodiments, the cancer is not cutaneous T-cell lymphomas, such as Sézary syndrome, CD30⁺ cutaneous lymphoma, and transformed mycosis fungoides.

In some embodiments, the KIR3DL1/2 or KLRC1/D1 antagonist is an anti-KIR3DL1/2 or anti-KLRC1/D1 antibody, or an antigen-binding fragment thereof. In one embodiment, the KIR3DL1/2 or KLRC1/D1 antagonist is an antibody or antigen-binding fragment thereof that specifically binds to KIR3DL1/2 or KLRC1/D1 and inhibits IGSF8 binding to KIR3DL1/2 or KLRC1/D1 (e.g., inhibits IGSF8 binding to KIR3DL1/2-mediated IFNγ secretion in NK cells by at least about 20%, 40%, 50%, 60%, 80%, 90% or more). In one embodiment, the anti-KIR3DL1/2 or anti-KLRC1/D1 antibody is a human antibody.

In certain embodiments, the anti-KIR3DL1/2 antibody or antigen-binding fragment thereof specifically binds to the D2 domain of KIR3DL1/2 and inhibits IGSF8 binding. In certain embodiments, the anti-KIR3DL1/2 antibody or antigen-binding fragment thereof specifically binds to an epitope within the D2 domain of KIR3DL1/2 and inhibits IGSF8 binding to residues S165, I171, and/or M186 of KIR3DL1/2. In one embodiment, the anti-KIR3DL2 antibody is not IPH4102.

In one embodiment, the KIR3DL1/2 antagonist is an extracellular domain (ECD) of IGSF8 that inhibits binding of IGSF8 to KIR3DL1/2, e.g., binding to residues S165, I171, and/or M186 of KIR3DL1/2, without triggering the inhibitory function of KIR3DL1/2 on NK cell activation, proliferation, and/or viability.

In one embodiment, the KIR3DL1/2 or KLRC1/D1 antagonist is a small molecule that binds to KIR3DL1/2 or KLRC1/D1 and inhibits binding of IGSF8 to KIR3DL1/2 or KLRC1/D1, e.g., binding to residues S165, I171, and/or M186 of KIR3DL1/2, without triggering the inhibitory function of KIR3DL1/2 on NK cell activation, proliferation, and/or viability.

In one embodiment, the KIR3DL1/2 antagonist is CpG-oligodeoxynucleotides (CpG-ODN), which, upon binding to the first (or D1) Ig-like domain in the ECD of KIR3DL1/2, causes KIR3DL1/2 down-modulation from the cell surface and translocation to the endosome to deliver the CpG-ODN to the toll-like receptor 9, and NK cell activation.

In a related aspect, the invention provides a use of an IGSF8 antagonist, an KIR3DL1 antagonist, an KIR3DL2 antagonist, or an KLRC1/D1 antagonist that inhibits IGSF8 binding to a receptor of IGSF8 selected from KIR3DL1, KIR3DL2, and KLRC1/D2 heterodimer, for treating cancer in a subject.

In certain embodiments, the use is for combination use with any one or more of a second therapeutic agent as described herein.

A related aspect of the invention provides a composition comprising an IGSF8 antagonist (e.g., an anti-IGSF8 monoclonal antibody or antigen-binding fragment thereof of the invention), an KIR3DL1 antagonist, an KIR3DL2 antagonist, or an KLRC1/D1 antagonist, that inhibits IGSF8 binding to a receptor of IGSF8 selected from KIR3DL1, KIR3DL2, and KLRC1/D2 heterodimer, for use in any of the methods of the invention described herein.

### 4. Routes of Administration and Carriers

In various embodiments, IGSF8 antagonists (e.g., an anti-IGSF8 monoclonal antibody or antigen-binding fragment thereof of the invention) and/or KIR3DL1/2 antagonist and/or KLRC1/D1 antagonists may be administered subcutaneously or intravenously.

In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in vivo by various routes, including, but not limited to, oral, intra-arterial, parenteral, intranasal, intramuscular, intracardiac, intraventricular, intratracheal, buccal, rectal, intraperitoneal, by inhalation, intradermal, topical, transdermal, and intrathecal, or otherwise, e.g., by implantation.

The subject compositions may be formulated into preparations in solid, semi-solid, liquid, or gaseous forms; including, but not limited to, tablets, capsules, powders, granules, ointments, solutions, suppositories, enemas, injections, inhalants, and aerosols.

In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist is delivered using gene therapy. As a non-limiting example, a nucleic acid molecule encoding IGSF8 antagonist (e.g., an anti-IGSF8 monoclonal antibody or antigen-binding fragment thereof of the invention) and/or KIR3DL1/2 antagonist and/or KLRC1/D1 (such as Cas9 and sgRNA, or Cas12a and crRNA) may be coated onto gold microparticles and delivered intradermally by a particle bombardment device, or “gene gun,” e.g., as described in the literature (see, e.g., Tang et al, Nature 356: 152-154 (1992)).

In various embodiments, compositions comprising IGSF8 antagonist (e.g., an anti-IGSF8 monoclonal antibody or antigen-binding fragment thereof of the invention) and/or KIR3DL1/2 antagonist and/or KLRC1/D1 antagonist are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, compositions comprising IGSF8 antagonist (e.g., an anti-IGSF8 monoclonal antibody or antigen-binding fragment thereof of the invention) and/or KIR3DL1/2 antagonist and/or KLRC1/D1 antagonist may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives.

In various embodiments, the compositions may be formulated for inhalation, for example, using pressurized acceptable propellants such as dichlorodifiuoromethane, propane, nitrogen, and the like.

The compositions may also be formulated, in various embodiments, into sustained release microcapsules, such as with biodegradable or non-biodegradable polymers. A non-limiting exemplary biodegradable formulation includes poly lactic acid-glycolic acid (PLGA) polymer. A non-limiting exemplary non-biodegradable formulation includes a polyglycerin fatty acid ester. Certain methods of making such formulations are described in, e.g., EP 1125584 A1.

Pharmaceutical dosage packs comprising one or more containers, each containing one or more doses of IGSF8 antagonist (e.g., an anti-IGSF8 monoclonal antibody or antigen-binding fragment thereof of the invention) and/or KIR3DL1/2 antagonist and/or KLRC1/D1 antagonist, are also provided. In some embodiments, a unit dosage is provided wherein the unit dosage contains a predetermined amount of a composition comprising IGSF8 antagonist (e.g., an anti-IGSF8 monoclonal antibody or antigen-binding fragment thereof of the invention) and/or KIR3DL1/2 antagonist and/or KLRC1/D1 antagonist, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated.

In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 50 μg/kg body weight to about 50 mg/kg body weight per dose. In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 100 μg/kg body weight to about 50 mg/kg body weight per dose. In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 100 μg/kg body weight to about 20 mg/kg body weight per dose. In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 0.5 mg/kg body weight to about 20 mg/kg body weight per dose.

In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 10 mg to about 1,000 mg per dose. In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 20 mg to about 500 mg per dose. In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 20 mg to about 300 mg per dose. In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist may be administered in an amount in the range of about 20 mg to about 200 mg per dose.

The IGSF8 antagonist (e.g., an anti-IGSF8 monoclonal antibody or antigen-binding fragment thereof of the invention) and/or KIR3DL1/2 antagonist and/or KLRC1/D1 antagonist compositions may be administered as needed to subjects. In some embodiments, an effective dose of IGSF8 antagonist (e.g., an anti-IGSF8 monoclonal antibody or antigen-binding fragment thereof of the invention) and/or KIR3DL1/2 antagonist and/or KLRC1/D1 antagonist is administered to a subject one or more times. In various embodiments, an effective dose of IGSF8 antagonist (e.g., an anti-IGSF8 monoclonal antibody or antigen-binding fragment thereof of the invention) and/or KIR3DL1/2 antagonist and/or KLRC1/D1 antagonist is administered to the subject once a month, less than once a month, such as, for example, every two months, every three months, or every six months. In other embodiments, an effective dose of IGSF8 antagonist (e.g., an anti-IGSF8 monoclonal antibody or antigen-binding fragment thereof of the invention) and/or KIR3DL1/2 antagonist is administered more than once a month, such as, for example, every two weeks, every week, twice per week, three times per week, daily, or multiple times per day. An effective dose of IGSF8 antagonist (e.g., an anti-IGSF8 monoclonal antibody or antigen-binding fragment thereof of the invention) and/or KIR3DL1/2 antagonist and/or KLRC1/D1 antagonist is administered to the subject at least once. In some embodiments, the effective dose of IGSF8 antagonist (e.g., an anti-IGSF8 monoclonal antibody or antigen-binding fragment thereof of the invention) and/or KIR3DL1/2 antagonist and/or KLRC1/D1 antagonist may be administered multiple times, including for periods of at least a month, at least six months, or at least a year. In some embodiments, IGSF8 antagonist (e.g., an anti-IGSF8 monoclonal antibody or antigen-binding fragment thereof of the invention) and/or KIR3DL1/2 antagonist and/or KLRC1/D1 antagonist is administered to a subject as-needed to alleviate one or more symptoms of a condition.

### 5. Combination Therapy

IGSF8 antagonists (e.g., an anti-IGSF8 monoclonal antibody or antigen-binding fragment thereof of the invention) and/or KIR3DL1/2 antagonist and/or KLRC1/D1 antagonist of the invention, including any antibodies and functional fragments thereof, may be administered to a subject in need thereof in combination with other biologically active substances or other treatment procedures for the treatment of diseases. For example, IGSF8 antagonists (e.g., an anti-IGSF8 monoclonal antibody or antigen-binding fragment thereof of the invention) and/or KIR3DL1/2 antagonists and/or KLRC1/D1 antagonist may be administered alone or with other modes of treatment. They may be provided before, substantially contemporaneous with, or after other modes of treatment, such as radiation therapy.

In some embodiments, the methods of the invention may comprise administering to the subject an effective amount of a second therapeutic agent comprising an immunotherapy, an immune checkpoint inhibitor, a cancer vaccine, a chimeric antigen receptor, a chemotherapeutic agent, a radiation therapy, an anti-angiogenesis agent, a growth inhibitory agent, an immune-oncology agent, an anti-neoplastic composition, a surgery, or a combination thereof.

For treatment of cancer, the IGSF8 antagonist (e.g., an anti-IGSF8 monoclonal antibody or antigen-binding fragment thereof of the invention) and/or KIR3DL1/2 antagonist and/or KLRC1/D1 antagonist may be administered in conjunction with one or more of anti-cancer agents, such as the immune checkpoint inhibitor, chemotherapeutic agent, growth inhibitory agent, anti-angiogenesis agent or anti-neoplastic composition.

In some embodiments, the immune checkpoint inhibitor is an antibody or antigen-binding fragment thereof specific for PD-1, PD-L1, PD-L2, LAG3, TIGIT, TIM3, NKG2A, CD276, VTCN1, VISR or HHLA2.

In some embodiments, the immune checkpoint inhibitor is an anti-PD-1 antibody, such as cemiplimab, nivolumab, or pembrolizumab.

In some embodiments, the immune checkpoint inhibitor is an anti-PD-L1 antibody, such as avelumab, durvalumab, atezolizumab, KN035, or CK-301.

In some embodiments, the immune checkpoint inhibitor is a (non-antibody) peptide inhibitor of PD-1/PD-L1, such as AUNP12; a small molecule inhibitor of PD-L1 such as CA-170, or a macrocyclic peptide such as BMS-986189.

In certain embodiments, IGSF8 antagonist (e.g., an anti-IGSF8 monoclonal antibody or antigen-binding fragment thereof of the invention) specifically binds to IGSF8 (an “IGSF8-binding antagonist”), e.g., IGSF8 antagonist antibody or antigen-binding fragment thereof, is administered with a second antagonist such as an immune checkpoint inhibitor (e.g., an inhibitor of the PD-1 or PD-L1 pathway), to a subject having a disease in which the stimulation of the immune system would be beneficial, e.g., cancer or infectious diseases. The two antagonists may be administered simultaneously or consecutively, e.g., as described below for the combination of IGSF8 antagonist with an immuno-oncology agent. One or more additional therapeutics, e.g., checkpoint modulators may be added to a treatment with IGSF8 binding antagonist for treating cancer or infectious diseases. In some embodiments, the IGSF8 antagonist is an antibody or antigen-binding fragment thereof that specifically binds to the D1 (Ig-V set domain) of IGSF8.

In certain embodiments, KIR3DL1/2 antagonist specifically binds to KIR3DL1/2 (an “KIR3DL1/2-binding antagonist”), e.g., KIR3DL1/2 antagonist antibody or antigen-binding fragment thereof, is administered with a second antagonist such as an immune checkpoint inhibitor (e.g., an inhibitor of the PD-1 or PD-L1 pathway), to a subject having a disease in which the stimulation of the immune system would be beneficial, e.g., cancer or infectious diseases. The two antagonists may be administered simultaneously or consecutively, e.g., as described below for the combination of KIR3DL1/2 antagonist with an immuno-oncology agent. One or more additional therapeutics, e.g., checkpoint modulators may be added to a treatment with KIR3DL1/2 binding antagonist for treating cancer or infectious diseases. In some embodiments, the KIR3DL1/2 antagonist is an antibody or an antigen-binding fragment thereof that specifically binds to the D2 (the middle Ig-like domain) of KIR3DL1/2, such as antibody or antigen-binding fragment that binds to S165, I171, and/or M186 of KIR3DL1/2, or inhibits IGSF8 binding via S165, I171, and/or M186.

In certain embodiments, KLRC1/D1 antagonist specifically binds to KLRC1/D1 (an “KLRC1/D1-binding antagonist”), e.g., KLRC1/D1 antagonist antibody or antigen-binding fragment thereof, is administered with a second antagonist such as an immune checkpoint inhibitor (e.g., an inhibitor of the PD-1 or PD-L1 pathway), to a subject having a disease in which the stimulation of the immune system would be beneficial, e.g., cancer or infectious diseases. The two antagonists may be administered simultaneously or consecutively, e.g., as described below for the combination of KLRC1/D1 antagonist with an immuno-oncology agent. One or more additional therapeutics, e.g., checkpoint modulators may be added to a treatment with KLRC1/D1 binding antagonist for treating cancer or infectious diseases.

In certain embodiments, IGSF8 antagonist (e.g., an anti-IGSF8 monoclonal antibody or antigen-binding fragment thereof of the invention) and/or KIR3DL1/2 antagonist and/or KLRC1/D1 antagonist is administered with another treatment, either simultaneously, or consecutively, to a subject, e.g., a subject having cancer. For example, IGSF8 antagonist (e.g., an anti-IGSF8 monoclonal antibody or antigen-binding fragment thereof of the invention) and/or KIR3DL1/2 antagonist and/or KLRC1/D1 antagonist may be administered with one of more of: radiotherapy, surgery, or chemotherapy, e.g., targeted chemotherapy or immunotherapy. Immunotherapy, e.g., cancer immunotherapy includes cancer vaccines and immuno-oncology agents.

IGSF8 antagonist (e.g., an anti-IGSF8 monoclonal antibody or antigen-binding fragment thereof of the invention) and/or KIR3DL1/2 antagonist and/or KLRC1/D1 antagonist may be, e.g., a protein, an antibody, antibody fragment or a small molecule, that binds to IGSF8 or KIR/3DL1/2 or KLRC1/D1, respectively. IGSF8 antagonist (e.g., an anti-IGSF8 monoclonal antibody or antigen-binding fragment thereof of the invention) and/or KIR3DL1/2 antagonist and/or KLRC1/D1 antagonist may be an antibody or antigen binding fragment thereof that specifically binds to IGSF8 or KIR3DL1/2 or KLRC1/D1, respectively.

In certain embodiments, a method of treatment of a subject having cancer comprises administering to the subject having the cancer IGSF8 antagonist (e.g., an anti-IGSF8 monoclonal antibody or antigen-binding fragment thereof of the invention) and/or KIR3DL1/2 antagonist and/or KLRC1/D1 antagonist, e.g., IGSF8 antibody and/or KIR3DL1/2 antibody and/or KLRC1/D1 antibody, and one or more immuno-oncology agents, such as immune checkpoint inhibitor.

Immunotherapy, e.g., therapy with an immuno-oncology agent, is effective to enhance, stimulate, and/or upregulate immune responses in a subject. In one aspect, the administration of IGSF8 antagonist (e.g., an anti-IGSF8 monoclonal antibody or antigen-binding fragment thereof of the invention) and/or KIR3DL1/2 antagonist and/or KLRC1/D1 antagonist with an immuno-oncology agent (such as a PD-1 inhibitor) has a synergic effect in the treatment of cancer, e.g., in inhibiting tumor growth.

In one aspect, IGSF8 antagonist (e.g., an anti-IGSF8 monoclonal antibody or antigen-binding fragment thereof of the invention) and/or KIR3DL1/2 antagonist and/or KLRC1/D1 antagonist is sequentially administered prior to administration of the immuno-oncology agent. In one aspect, IGSF8 antagonist (e.g., an anti-IGSF8 monoclonal antibody or antigen-binding fragment thereof of the invention) and/or KIR3DL1/2 antagonist and/or KLRC1/D1 antagonist is administered concurrently with the immunology-oncology agent (such as PD-1 inhibitor). In yet one aspect, IGSF8 antagonist (e.g., an anti-IGSF8 monoclonal antibody or antigen-binding fragment thereof of the invention) and/or KIR3DL1/2 antagonist and/or KLRC1/D1 antagonist is sequentially administered after administration of the immuno-oncology agent (such as PD-1 inhibitor).

The administration of the two agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In certain aspects, IGSF8 antagonist (e.g., an anti-IGSF8 monoclonal antibody or antigen-binding fragment thereof of the invention) and/or KIR3DL1/2 antagonist and/or KLRC1/D1 antagonist and an immuno-oncology agent (e.g., PD-1 inhibitor) are administered simultaneously, e.g., are infused simultaneously, e.g., over a period of 30 or 60 minutes, to a patient. IGSF8 antagonist may be co-formulated with an immuno-oncology agent (such as PD-1 inhibitor).

Immuno-oncology agents include, for example, a small molecule drug, antibody or fragment thereof, or other biologic or small molecule. Examples of biologic immuno-oncology agents include, but are not limited to, antibodies, antibody fragments, vaccines and cytokines. In one aspect, the antibody is a monoclonal antibody. In certain aspects, the monoclonal antibody is humanized or human antibody.

In one aspect, the immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on immune cells, e.g., T cells, both of which result in amplifying antigen-specific T cell responses. In certain aspects, an immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on cells involved in innate immunity, e.g., NK cells, and wherein the immuno-oncology agent enhances innate immunity. Such immuno-oncology agents are often referred to as immune checkpoint regulators, e.g., immune checkpoint inhibitor or immune checkpoint stimulator.

In certain embodiments, an immuno-oncology agent targets a stimulatory or inhibitory molecule that is a member of the immunoglobulin super family (IgSF). For example, an immuno-oncology agent may be an agent that targets (or binds specifically to) a member of the B7 family of membrane-bound ligands, which includes B7-1, B7-2, B7-H1 (PD-L1), B7-DC (PD-L2), B7-H2 (ICOS-L), B7-H3, B7-H4, B7-H5, and B7-H6, or a co-stimulatory or co-inhibitory receptor binding specifically to a B7 family member. An immuno-oncology agent may be an agent that targets a member of the TNF family of membrane bound ligands or a co-stimulatory or co-inhibitory receptor binding specifically thereto, e.g., a TNF receptor family member. Exemplary TNF and TNFR family members that may be targeted by immuno-oncology agents include CD40 and CD40L, OX-40, OX-40L, GITR, GITRL, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTfiR, LIGHT, DcR3, HVEM, VEGI/TL1A, TRAMP/DR3, EDAR, EDA1, XEDAR, EDA2, TNFR1, Lymphotoxin α/TNPβ, TNFR2, TNFα, LTfiR, Lymphotoxin a 1β2, FAS, FASL, RELT, DR6, TROY and NGFR. An immuno-oncology agent that may be used in combination with IGSF8 antagonist agent for treating cancer may be an agent, e.g., an antibody, targeting an IgSF member, such as a B7 family member, a B7 receptor family member, a TNF family member or a TNFR family member, such as those described above.

In one aspect, IGSF8 antagonist (e.g., an anti-IGSF8 monoclonal antibody or antigen-binding fragment thereof of the invention) and/or KIR3DL1/2 antagonist and/or KLRC1/D1 antagonist is administered with one or more of (i) an antagonist of a protein that inhibits T cell activation (e.g., immune checkpoint inhibitor) such as CTLA-4, PD-1, PD-L1, PD-L2, LAG-3, TIM3, Galectin 9, CEACAM-1, BTLA, CD69, Galectin-1, TIGIT, CD113, GPR56, VISTA, B7-H3, B7-H4, 2B4, CD48, GARP, PDIH, LAIR1, TIM-1, TIM-4, and PSGL-1 and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, CD40L, DR3 and CD28H.

In one aspect, an immuno-oncology agent is an agent that inhibits (i.e., an antagonist of) a cytokine that inhibits T cell activation (e.g., IL-6, IL-10, TGF-β, VEGF, and other immunosuppressive cytokines) or is an agonist of a cytokine, such as IL-2, IL-7, IL-12, IL-15, IL-21 and IFNα (e.g., the cytokine itself) that stimulates T cell activation, and stimulates an immune response.

Other agents that can be combined with IGSF8 antagonist (e.g., an anti-IGSF8 monoclonal antibody or antigen-binding fragment thereof of the invention) and/or KIR3DL1/2 antagonist and/or KLRC1/D1 antagonist for stimulating the immune system, e.g., for the treatment of cancer and infectious diseases, include antagonists of inhibitory receptors on NK cells or agonists of activating receptors on NK cells. For example, anti-IGSF8 antagonist (e.g., an anti-IGSF8 monoclonal antibody or antigen-binding fragment thereof of the invention) can be combined with an antagonist of KIR, such as an KIR3DL1/2 antagonist, and/or an antagonist against KLRC1/D1.

Yet other agents for combination therapies include agents that inhibit or deplete macrophages or monocytes, including but not limited to CSF-IR antagonists such as CSF-IR antagonist antibodies including RG7155 (WOl1/70024, WOl1/107553, WO11/131407, W013/87699, W013/119716, WO13/132044) or FPA008 (WOl 1/140249; W013169264; WO14/036357).

Immuno-oncology agents also include agents that inhibit TGF-β signaling.

Additional agents that may be combined with IGSF8 antagonist (e.g., an anti-IGSF8 monoclonal antibody or antigen-binding fragment thereof of the invention) and/or KIR3DL1/2 antagonist and/or KLRC1/D1 antagonist include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Yet other therapies that may be combined with IGSF8 antagonist (e.g., an anti-IGSF8 monoclonal antibody or antigen-binding fragment thereof of the invention) and/or KIR3DL1/2 antagonist and/or KLRC1/D1 antagonist include therapies that deplete or block Treg cells, e.g., an agent that specifically binds to CD25.

Another therapy that may be combined with IGSF8 antagonist (e.g., an anti-IGSF8 monoclonal antibody or antigen-binding fragment thereof of the invention) and/or KIR3DL1/2 antagonist and/or KLRC1/D1 antagonist is a therapy that inhibits a metabolic enzyme such as indoleamine dioxigenase (IDO), dioxigenase, arginase, or nitric oxide synthetase.

Another class of agents that may be used includes agents that inhibit the formation of adenosine or inhibit the adenosine A2A receptor.

Other therapies that may be combined with IGSF8 antagonist and/or KIR3DL1/2 antagonist and/or KLRC1/D1 antagonist for treating cancer include therapies that reverse/prevent T cell anergy or exhaustion and therapies that trigger an innate immune activation and/or inflammation at a tumor site.

IGSF8 antagonist (e.g., an anti-IGSF8 monoclonal antibody or antigen-binding fragment thereof of the invention) and/or KIR3DL1/2 antagonist and/or KLRC1/D1 antagonist may be combined with each other, and/or with more than one immuno-oncology agent (such as immune checkpoint inhibitor), and may be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Treg or other immune suppressing cells; a therapy that stimulates positive immune regulation, e.g., with agonists that stimulate the CD-137, OX-40 and/or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase or nitric oxide synthetase; a therapy that reverses/prevents T cell anergy or exhaustion; a therapy that triggers an innate immune activation and/or inflammation at a tumor site; administration of immune stimulatory cytokines or blocking of immuno repressive cytokines.

For example, IGSF8 antagonist (e.g., an anti-IGSF8 monoclonal antibody or antigen-binding fragment thereof of the invention) and/or KIR3DL1/2 antagonist and/or KLRC1/D1 antagonist can be used with one or more agonistic agents that ligate positive costimulatory receptors; one or more antagonists (blocking agents) that attenuate signaling through inhibitory receptors, such as antagonists that overcome distinct immune suppressive pathways within the tumor microenvironment (e.g., block PD-L1/PD-1/PD-L2 interactions); one or more agents that increase systemically the frequency of anti-tumor immune cells, such as T cells, deplete or inhibit Tregs (e.g., by inhibiting CD25); one or more agents that inhibit metabolic enzymes such as IDO; one or more agents that reverse/prevent T cell anergy or exhaustion; and one or more agents that trigger innate immune activation and/or inflammation at tumor sites.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist and an immuno-oncology agent, wherein the immuno-oncology agent is a CTLA-4 antagonist, such as an antagonistic CTLA-4 antibody. Suitable CTLA-4 antibodies include, for example, YERVOY (ipilimumab) or tremelimumab.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011). Another approach to target the PD-1 receptor is the recombinant protein composed of the extracellular domain of PD-L2 (B7-DC) fused to the Fc portion of IgGl, called AMP-224.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a PD-L1 antagonist, such as an antagonistic PD-L1 antibody. Suitable PD-L1 antibodies include, for example, MPDL3280A (RG7446; WO2010/077634), durvalumab (MEDI4736), BMS-936559 (WO2007/005874), MSB0010718C (WO2013/79174) or rHigM12B7.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a LAG-3 antagonist, such as an antagonistic LAG-3 antibody. Suitable LAG3 antibodies include, for example, BMS-986016 (WO10/19570, WO 14/08218), or IMP-731 or IMP-321 (WO08/132601, WO09/44273).

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a CD137 (4-1BB) agonist, such as an agonistic CD137 antibody. Suitable CD137 antibodies include, for example, urelumab or PF-05082566 (WO12/32433).

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a GITR agonist, such as an agonistic GITR antibody. Suitable GITR antibodies include, for example, TRX-518 (WO06/105021, WO09/009116), MK-4166 (WO 11/028683) or a GITR antibody disclosed in WO2015/031667.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is an OX40 agonist, such as an agonistic OX40 antibody. Suitable OX40 antibodies include, for example, MEDI-6383, MEDI-6469 or MOXR0916 (RG7888; WO06/029879).

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a CD40 agonist, such as an agonistic CD40 antibody. In certain embodiments, the immuno-oncology agent is a CD40 antagonist, such as an antagonistic CD40 antibody. Suitable CD40 antibodies include, for example, lucatumumab (HCD122), dacetuzumab (SGN-40), CP-870,893 or Chi Lob 7/4.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a CD27 agonist, such as an agonistic CD27 antibody. Suitable CD27 antibodies include, for example, varlilumab (CDX-1127).

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is MGA271 (to B7H3) (WOl 1/109400).

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a KIR antagonist, such as lirilumab.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is an IDO antagonist. Suitable IDO antagonists include, for example, INCB-024360 (WO2006/122150, WO07/75598, WO08/36653, WO08/36642), indoximod, NLG-919 (WO09/73620, WO09/1156652, WOl 1/56652, WO 12/142237) or F001287.

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein the immuno-oncology agent is a Toll-like receptor agonist, e.g., a TLR2/4 agonist (e.g., Bacillus Calmette-Guerin); a TLR7 agonist (e.g., Hiltonol or Imiquimod); a TLR7/8 agonist (e.g., Resiquimod); or a TLR9 agonist (e.g., CpG7909).

In one embodiment, a subject having a disease that may benefit from stimulation of the immune system, e.g., cancer or an infectious disease, is treated by administration to the subject of IGSF8 antagonist (e.g., an anti-IGSF8 monoclonal antibody or antigen-binding fragment thereof of the invention) and/or KIR3DL1/2 antagonist and/or KLRC1/D1 antagonist, and an immuno-oncology agent, wherein, the immuno-oncology agent is a TGF-β inhibitor, e.g., GC1008, LY2157299, TEW7197 or IMC-TR1.

Another therapy that may be combined with IGSF8 antagonist (e.g., an anti-IGSF8 monoclonal antibody or antigen-binding fragment thereof of the invention) and/or KIR3DL1/2 antagonist and/or KLRC1/D1 antagonist is a therapeutic antibody, such as one that is efficacious to treat cancer. Exemplary but non-limiting therapeutic antibodies include: 3F8, 8H9, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Amivantamab, Anatumomab mafenatox, Andecaliximab, Anetumab ravtansine, Anifrolumab, Anrukinzumab, Apolizumab, Aprutumab ixadotin, Arcitumomab, Ascrinvacumab, Aselizumab, Atezolizumab, Atidortoxumab, Atinumab, Atorolimumab, Avelumab, Azintuxizumab vedotin, Bapineuzumab, Basiliximab, Bavituximab, BCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, BirtamimabBivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab, Carlumab, Carotuximab, Catumaxomab, cBR-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, GancotamabGanitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ianalumab, Ibalizumab, IBID308, Ibritumomab tiuxetan, Icrucumab, Idarucizumab, Ifabotuzumab, Igovomab, Iladatuzumab vedotin, IMAB363, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Mapatumumab, Margetuximab, MarstacimabMaslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, OtilimabOtlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, RanevetmabRanibizumab, Raxibacumab, Ravagalimab, Ravulizumab, Refanezumab, Regavirumab, REGN-EB, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Talquetamab, Tamtuvetmab, Tanezumab, Taplitumomab paptox, Tarextumab, TavolimabTeclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, tiragolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab, Zolbetuximab, (=IMAB362, Claudiximab), Zolimomab aritox, or combination thereof.

### 6. Exemplary IGSF8 Antagonists

In some embodiments, an IGSF8 antagonist is an IGSF8 antibody. In some embodiments, an IGSF8 antagonist for treating cancer may be a non-antibody protein, such as a soluble IGSF8 or a portion thereof (e.g., the ECD) that inhibits the interaction between IGSF8 and its ligand, optionally further comprising a fusion partner and in the form of a fusion molecule.

In some embodiments, the IGSF8 antagonist is a soluble ECD of KIR3DL1/2, such as the D2 domain of KIR3DL1/2 or a fragment thereof that binds to IGSF8, which may optionally further comprise a fusion partner, such as a sequence tag (e.g., His tag, FLAG tag, etc). Such IGSF8 antagonists may bind to IGSF8 and blocks its binding to the KIR3DL1/2 receptors on NK cells, thus blocking IGSF8-mediated down-regulation of NK cell activity and/or viability.

In some embodiments, the IGSF8 antagonist is a soluble ECD of KLRC1/D1, such as the ECD of KLRC1 or KLRD1, or a fragment thereof that binds to IGSF8, which may optionally further comprise a fusion partner, such as a sequence tag (e.g., His tag, FLAG tag, etc). Such IGSF8 antagonists may bind to IGSF8 and blocks its binding to the KLRC1/D1 receptors on NK cells, thus blocking IGSF8-mediated down-regulation of NK cell activity and/or viability.

The antagonist, in other embodiments, may also be a small molecule or small peptide.

### IGSF8 Antibodies

One aspect of the invention provides a monoclonal antibody specific for IGSF8. In certain embodiments, the monoclonal antibody is specific for the extracellular domain (ECD) of IGSF8. In certain embodiments, the monoclonal antibody is specific for the Ig-V set extracellular domain (D1 domain) of IGSF8. In some embodiments, antibodies that block binding of IGSF8 and its ligand are provided. In certain embodiments, the monoclonal antibody inhibits IGSF8 binding to KIR3DL2 and/or KIR3DL1, such as inhibiting IGSF8 binding to residues S165, I171, and/or M186. In certain embodiments, the monoclonal antibody inhibits IGSF8 binding to KLRC1/D1. In certain embodiments, the monoclonal antibody has cross-species reactivity, e.g., the monoclonal antibody binds both human and mouse IGSF8. In certain embodiments, the monoclonal antibody is specific for human IGSF8. In some embodiments, IGSF8 antibody inhibits IGSF8-mediated signaling. In certain embodiments, the monoclonal antibody competes with any one of the anti-IGSF8 antibodies disclosed herein for binding to IGSF8. In certain embodiments, the monoclonal antibody binds the same epitope on IGSF8 as any one of the anti-IGSF8 antibodies disclosed herein.

In some embodiments, IGSF8 antibody of the invention has a dissociation constant (K_{d}) of ≤1 M, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M) for IGSF8, e.g., for human IGSF8. In certain embodiments, IGSF8 antibody has a dissociation constant (K_{d}) of ≤1 M, ≤100 nM, ≤1 nM, ≤1 nM, ≤0.1 nM, ≤0.01 nM, or 0.001 nM (e.g. 10⁻⁸ M or less, e.g. from 10⁻⁸ M to 10⁻¹³ M, e.g., from 10⁻⁹ M to 10⁻¹³ M) for IGSF8, e.g., for human IGSF8.

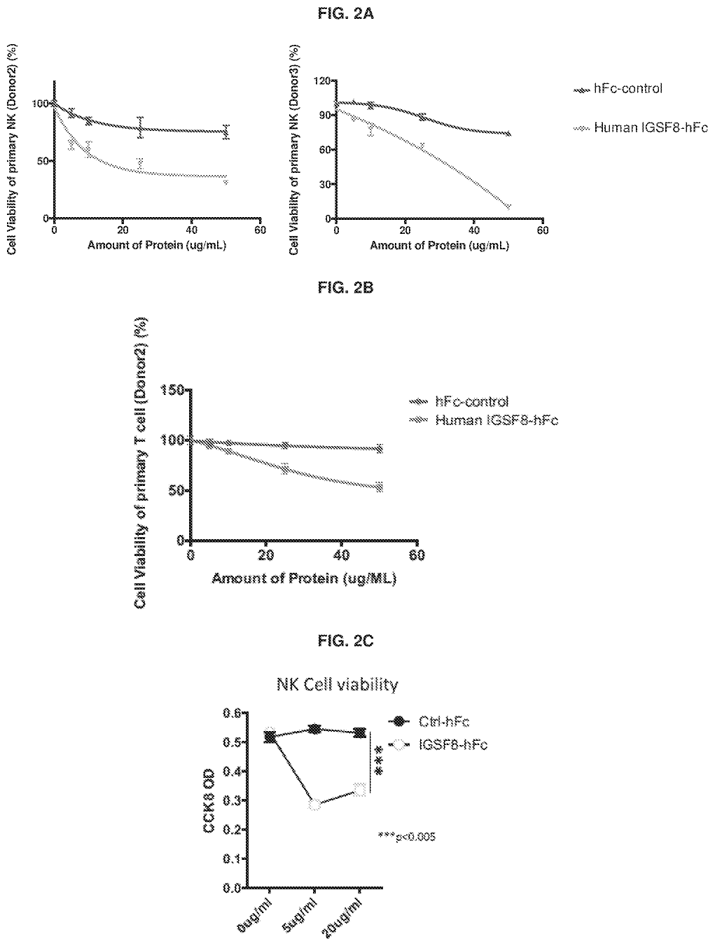

In some embodiments, an IGSF8 antibody having any of the characteristics provided herein inhibits at least 25%, 50%, 75%, 80%, 90% or 100% of the signaling of IGSF8, e.g., signaling through KIR3DL1/2 and/or KLRC1/D1. For example, KIR3DL1/2 and/or KLRC1/D1 signaling upon binding to IGSF8 can be assayed in NK cells based on IFNγ secretion, which can be analyzed using standard techniques such as ELISA. In some embodiments, the IGSF8 antibody inhibits signaling in NK cells, such as in any one of the signaling pathways described in FIG. 2D (e.g., cell cycle, DNA replication, etc) or FIG. 2E (e.g., PRF1, GZMB, or GZMA).

In some embodiments, an IGSF8 antibody of the invention includes any one of antibodies described herein, including C1-C39, or C30-C39, as described in Example 7, as well as antibodies L1-01 to L1-033, and L2-01 to L2-010, as described in Example 24 (all incorporated herein by reference), as well as any of the antibodies described in this section.

Unless explicitly indicated, all antibody and CDR sequences are based on IMGT numbering scheme, except that C1-C29 are annotated by the Kabat numbering scheme (while others, such as those based on C30-C39 and L1/L2 derivatives are based on the IMGT numbering scheme). In addition, heavy chain only sequence consensus/motifs after C39, as well as CDR sequences in the CDR region mutant analysis (L1/L2 derivatives), are also based on IMGT numbering scheme.

Using the HCVR CDR1-3 sequences of the high affinity anti-IGSF8 antibodies C30-C39 as query sequences, numerous similar CDR sequences were identified in proprietary human antibody libraries, and antibodies having such small CDR variations are also anti-IGSF8 antibodies of the invention specific for IGSF8 (e.g., specific for the Ig-V set domain or the D1 domain of the ECD of IGSF8).

Similarly, using the LCVR CDR1-3 sequences of the high affinity anti-IGSF8 antibodies C30-C39 as query sequences, numerous similar CDR sequences were identified in proprietary human antibody libraries, and antibodies having such small CDR variations are also anti-IGSF8 antibodies of the invention specific for IGSF8 (e.g., specific for the Ig-V set domain or the D1 domain of the ECD of IGSF8).

Thus, in some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 469, 470 and 471, respectively, which are similar to and encompass the HCVR CDR1-3 of monoclonal antibody C30/B34; and/or (b) a light chain variable region (LCVR) comprising the LCVR CDR1, CDR2, and CDR3 of SEQ ID NOs: 562, 563 and 564, respectively, which are similar to and encompass the LCVR CDR1-3 of monoclonal antibody C30/B34.

SEQ ID NO: 469: G Xaa1 Xaa2 Xaa3 Xaa4 Xaa5 Xaa6 A, wherein Xaa1=F or Y; Xaa2=S or T; Xaa3=L, F or I; Xaa4=R, S or I; Xaa5=D or S; and Xaa6=Y or S.

SEQ ID NO: 470: I Xaa1 GSGG Xaa2 T, wherein Xaa1=S or T, and Xaa2=N or S.

SEQ ID NO: 471: Xaa1 Xaa2 Xaa3 Xaa4 Xaa5 Xaa6 Xaa7 Xaa8, wherein Xaa1=E, A or S, Xaa2=R, L or S, Xaa3=W, A, Y, V, G or S, Xaa4=R, L or S, Xaa5=L, Y, P, T, I, N, K, H or Q, Xaa6=L, V, F, I, G, R or H, Xaa7=A, Y, V or any acidic residue (D/E), and Xaa8=Y, A, T, P, K, S or Q.

SEQ ID NO: 562: Xaa1 Xaa2 Xaa3 H Xaa4 Y, wherein Xaa1=K, Q, P or H, Xaa2=S, V, I or R, Xaa3=N, S, L, I or M, Xaa4=K, N or T,

SEQ ID NO: 563: AAS, and,

SEQ ID NO: 564: Xaa1 Xaa2 Xaa3 Xaa4 Xaa5 Xaa6 P Xaa7 Xaa8, wherein Xaa1=L, Q, K or H, Xaa2=L, Q, K or H, Xaa3=S, I or R, Xaa4=Y or F, Xaa5=P, N, S or T, Xaa6=P, N, S or T, Xaa7=L, I or R, Xaa8=P, N, S or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 472, 473 and 474, respectively, which are similar to and encompass the HCVR CDR1-3 of monoclonal antibody C31/B46; and/or (b) a light chain variable region (LCVR) comprising the LCVR CDR1, CDR2, and CDR3 of SEQ ID NOs: 565, 566 and 567, respectively, which are the LCVR CDR1-3 of monoclonal antibody C31/B46.

SEQ ID NO: 472:
GFTFSTYG,
SEQ ID NO: 473:
IWDDGSYK,
and,
SEQ ID NO: 474:
A Xaa1 GYS Xaa2 S Xaa3 Xaa4 A Xaa5, wherein
Xaa1 = V or G, Xaa2 = D or Y, Xaa3 = Y, D or S,
Xaa4 = R, L or M, Xaa5 = L, I or S.
SEQ ID NO: 565:
QGISTF,
SEQ ID NO: 566:
AAS,
and,
SEQ ID NO: 567:
QQTYSTQWT.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 475, 476 and 477, respectively, which are similar to and encompass the HCVR CDR1-3 of monoclonal antibody C32/B104; and/or (b) a light chain variable region (LCVR) comprising the LCVR CDR1, CDR2, and CDR3 of SEQ ID NOs: 568, 569 and 570, respectively, which are LCVR CDR1-3 of monoclonal antibody C32/B104.

SEQ ID NO: 475: GYTFTNDI,
SEQ ID NO: 476: INAGYGNT,
and,
SEQ ID NO: 477: ARGYYRSPTW Xaa1 D Xaa2, wherein
Xaa1 =F or I, and Xaa2 = W or Y.
SEQ ID NO: 568: QSISSW,
SEQ ID NO: 569: KAS,
and,
SEQ ID NO: 570: QQYGDYPYT.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 478, 479 and 480, respectively, which are similar to and encompass the HCVR CDR1-3 of monoclonal antibody C33/1C2; and/or (b) a light chain variable region (LCVR) comprising the LCVR CDR1, CDR2, and CDR3 of SEQ ID NOs: 571, 572 and 573, respectively, which are LCVR CDR1-3 of monoclonal antibody C33/1C2.

SEQ ID NO: 478: GFTFSTYG,

SEQ ID NO: 479: IWDDGSYK, and,

SEQ ID NO: 480: ARD Xaa1 S Xaa2 W Xaa3 YAFD Xaa4, wherein Xaa1=G or C, Xaa2=V or G, Xaa3=V or G, and Xaa4=L or I.

SEQ ID NO: 571: Xaa1 D Xaa2 Xaa3 Xaa4 Y, wherein Xaa1=K, Q, P or H, Xaa2=S, N, I or L, Xaa3=S, I or R, Xaa4=any acidic residue (D/E).

SEQ ID NO: 572: DAA, and,

SEQ ID NO: 573: Xaa1 Xaa2 Xaa3 Xaa4 Xaa5 Xaa6 Xaa7 Xaa8 Xaa9, wherein Xaa1=L, Q, K or H, Xaa2=Q, K, H or L, Xaa3=Y, S, D or F, Xaa4=V, A or any acidic residue (D/E), Xaa5=S, I or R, Xaa6=L, F or V, Xaa7=H, P or T, Xaa8=Y, S, F or D, Xaa9=P, N, S or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 481, 482 and 483, respectively, which are similar to and encompass the HCVR CDR1-3 of monoclonal antibody C34/1D7; and/or (b) a light chain variable region (LCVR) comprising the LCVR CDR1, CDR2, and CDR3 of SEQ ID NOs: 574, 575 and 576, respectively, which are similar to and encompass the LCVR CDR1-3 of monoclonal antibody C34/1D7.

SEQ ID NO: 481: GFT Xaa1 Xaa2 S Xaa3 A, wherein Xaa1=V or F, Xaa2=N or S, and Xaa3=F or Y,

SEQ ID NO: 482: I Xaa1 GSGG Xaa2 T, wherein Xaa1=S or T, & Xaa2=S or G, and,

SEQ ID NO: 483: AR Xaa1 V Xaa2 GYGAF Xaa3 Xaa4, wherein Xaa1=any acidic residue (D/E), Xaa2=any acidic residue (D/E), Xaa3=A or any acidic residue (D/E), and Xaa4=L or I.

SEQ ID NO: 574: Xaa1 Xaa2 Xaa3 Xaa4 Xaa5 Y, wherein Xaa1=Q, P or any basic residue (R/H/K), Xaa2=S, N or T, Xaa3=N, S, L, I or M, Xaa4=H, R, I or S, Xaa5=H, N, D, S, K, T or I,

SEQ ID NO: 575: GAS, and,

SEQ ID NO: 576: Xaa1 Xaa2 Xaa3 Xaa4 Xaa5 Xaa6 Xaa7 Xaa8 Xaa9, wherein Xaa1=H, Q, K, L or P, Xaa2=H, E, Q, K, L or P, Xaa3=N, A, S, T or P, Xaa4=Y, S, V, L or F, Xaa5=S, I or R, Xaa6=V, A or any acidic residue (D/E), Xaa7=A, Q, K, R, T or P, Xaa8=Y or F, Xaa9=P, N, S or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 484, 485 and 486, respectively, which are similar to and encompass the HCVR CDR1-3 of monoclonal antibody C35/1B1; and/or (b) a light chain variable region (LCVR) comprising the LCVR CDR1, CDR2, and CDR3 of SEQ ID NOs: 577, 578 and 579, respectively, which are the LCVR CDR1-3 of monoclonal antibody C35/1B1.

SEQ ID NO: 484: GFTF Xaa1 Xaa2 Xaa3 A, wherein Xaa1=R, N or S, Xaa2=D or S, and Xaa3=F or Y,

SEQ ID NO: 485: I Xaa1 GSGG Xaa2 T, wherein Xaa1=S or T, & Xaa2=N, S or G,

SEQ ID NO: 486: A Xaa1 Xaa2 GWE Xaa3 RTPG Xaa4 Xaa5 D Xaa6, wherein Xaa1=R or S, Xaa2=V or any acidic residue (D/E), Xaa3=V or G, Xaa4=D or Y, Xaa5=L, F or I, and Xaa6=D, Y, H or S.

SEQ ID NO: 577: HRIFSY,
SEQ ID NO: 578: GAS,
and,
SEQ ID NO: 579: QQSFSDPYT.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 487, 488 and 489, respectively, which are similar to and encompass the HCVR CDR1-3 of monoclonal antibody C36/1B4′ and/or (b) a light chain variable region (LCVR) comprising the LCVR CDR1, CDR2, and CDR3 of SEQ ID NOs: 580, 581 and 582, respectively, which are similar to and encompass the LCVR CDR1-3 of monoclonal antibody C36/1B4.

SEQ ID NO: 487: GFTFSS Xaa1 A, wherein Xaa1=Y or S, SEQ ID NO: 488: ITGSGGST, and,

SEQ ID NO: 489: AR Xaa1 Xaa2 Xaa3 Xaa4 L Xaa5 Xaa6, wherein Xaa1=D or G, Xaa2=R or absent, Xaa3=G or C, Xaa4=A, G or S, Xaa5=any acidic residue (D/E), and Xaa6=L, Y, I or V.

SEQ ID NO: 580: Xaa1 Xaa2 Xaa3 H Xaa4 Y, wherein Xaa1=K, Q, P or H, Xaa2=S, V, I or R, Xaa3=N, S, L, I or M, Xaa4=K, N or T,

SEQ ID NO: 581: SAS, and,

SEQ ID NO: 582: Xaa1 Xaa2 Xaa3 Xaa4 Xaa5 Xaa6 P Xaa7 Xaa8, wherein Xaa1=L, Q, K or H, Xaa2=L, Q, K or H, Xaa3=S, I or R, Xaa4=Y or F, Xaa5=P, N, S or T, Xaa6=P, N, S or T, Xaa7=L, I or R, Xaa8=P, N, S or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 490, 491 and 492, respectively, which are similar to and encompass the HCVR CDR1-3 of monoclonal antibody C37/3F12; and/or (b) a light chain variable region (LCVR) comprising the LCVR CDR1, CDR2, and CDR3 of SEQ ID NOs: 583, 584 and 585, respectively, which are similar to and encompass the LCVR CDR1-3 of monoclonal antibody C37/3F12.

SEQ ID NO: 490: GFTFSSYS,

SEQ ID NO: 491: ISSSSSYI,

SEQ ID NO: 492: Xaa1 R Xaa2 Xaa3 Xaa4 Xaa5 Xaa6 Xaa7 Xaa8 Xaa9 D Xaa10 Xaa1 1 Xaa12 Xaa13, wherein Xaa1=C or G, Xaa2=P or Q, Xaa3=Y or D, Xaa4=Y, A or any acidic residue (D/E), Xaa5=F or L, Xaa6=W or L, Xaa7=S, R or I, Xaa8=C, V or G, Xaa9=W, C or L, Xaa10=W, C or G, Xaa11=Y, F or V, Xaa12=D or A, and Xaa13=H, P or T.

SEQ ID NO: 583: Xaa1 Xaa2 Xaa3 Xaa4 Xaa5 Xaa6, wherein Xaa1=Q, L, P or any basic residue (R/H/K), Xaa2=D, S, G, R, T or I, Xaa3=N, S, L, V, T or I, Xaa4=H, N, S, G, R, T or I, Xaa5=N, A, S, E, T, P or I, Xaa6=Q, D, S or Y,

SEQ ID NO: 584: DAS, and,

SEQ ID NO: 585: Xaa1 Xaa2 Xaa3 Xaa4 Xaa5 Xaa6 Xaa7 Xaa8 Xaa9 Xaa10, wherein Xaa1=N, E, Q, L, P or any basic residue (R/H/K), Xaa2=N, E, Q, L, P or any basic residue (R/H/K), Xaa3=S, G, R, T or I, Xaa4=Y, H, D, S or F, Xaa5=S, G, R, T, I or M, Xaa6=N, A, S, T, P or I, Xaa7=H, L, V, R or I, Xaa8=A, S, Q, T, P or any basic residue (R/H/K), Xaa9=Y, H, N, S, F or any acidic residue (D/E), Xaa10=N, A, S, T or P.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 493, 494 and 495, respectively, which are similar to and encompass the HCVR CDR1-3 of monoclonal antibody C38/2B4; and/or (b) a light chain variable region (LCVR) comprising the LCVR CDR1, CDR2, and CDR3 of SEQ ID NOs: 586, 587 and 588, respectively, which are similar to and encompass the LCVR CDR1-3 of monoclonal antibody C38/2B4.

SEQ ID NO: 493: GFT Xaa1 Xaa2 Xaa3 Xaa4 A, wherein Xaa1=F or C, Xaa2=R, N or S, Xaa3=D or S, and Xaa4=F or Y,

SEQ ID NO: 494: I Xaa1 GSGG Xaa2 T, wherein Xaa1=S or T, & Xaa2=N, S or G,

SEQ ID NO: 495: Xaa1 Xaa2 Xaa3 Xaa4 Xaa5 Xaa6 Xaa7 Xaa8 Xaa9 Xaa10 Xaa1 1 Xaa12 Xaa13 Xaa14 Xaa15, wherein Xaa1=E, A or S, Xaa2=R, S or I, Xaa3=V or G, Xaa4=A or any acidic residue (D/E), Xaa5=D, Y or S, Xaa6=Y or S, Xaa7=R, S or I, Xaa8=V, G or C, Xaa9=L, W, G or C, Xaa10=P, H or T, Xaa1 1=R, S or I, Xaa12=L, W, C, G or R, Xaa13=L, F, V or C, Xaa14=Y, D or A, and Xaa15=P, H, S or T.

SEQ ID NO: 586: Xaa1 Xaa2 Xaa3 Xaa4 Xaa5 Xaa6, wherein Xaa1=Q, R or L, Xaa2=A, S, N or T, Xaa3=V, F or L, Xaa4=G or D, Xaa5=A, S, K, T or P, Xaa6=Y, L, V, F or I,

SEQ ID NO: 587: GVS, and

SEQ ID NO: 588: Xaa1 Xaa2 Xaa3 Xaa4 Xaa5 Xaa6 P Xaa7 Xaa8, wherein Xaa1=K, Q, L or H, Xaa2=Q, K, H or L, Xaa3=S, I, T or R, Xaa4=N, H, D or Q, Xaa5=V, A or any acidic residue (D/E), Xaa6=A, G, V, L or F, Xaa7=G, R or L, Xaa8=K, S, P or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises: (a) a heavy chain variable region (HCVR) comprising the HCVR CDR1, CDR2, and CDR3 of SEQ ID NOs: 496, 497 and 498, respectively, which are similar to and encompass the HCVR CDR1-3 of monoclonal antibody C39/8G4; and/or (b) a light chain variable region (LCVR) comprising the LCVR CDR1, CDR2, and CDR3 of SEQ ID NOs: 589, 590 and 591, respectively, which are similar to and encompass the LCVR CDR1-3 of monoclonal antibody C39/8G4.

SEQ ID NO: 496: GFTFSSYA,

SEQ ID NO: 497: ITGSGGST, and,

SEQ ID NO: 498: Xaa1 Xaa2 Xaa3 Xaa4 Xaa5 Xaa6 D Xaa7, wherein Xaa1=A or W, Xaa2=F, P, R or Y, Xaa3=D, H, P or S, Xaa4=R or S, Xaa5=D, I or N, Xaa6=L or P, and Xaa7=S or W.

SEQ ID NO: 589: Xaa1 Xaa2 Xaa3 H Xaa4 Y, wherein Xaa1=K, Q, P or H, Xaa2=S, V, I or R, Xaa3=N, S, L, I or M, Xaa4=K, N or T,

SEQ ID NO: 590: AAS, and,

SEQ ID NO: 591: Xaa1 Xaa2 Xaa3 Xaa4 Xaa5 Xaa6 P Xaa7 Xaa8, wherein Xaa1=L, Q, K or H, Xaa2=L, Q, K or H, Xaa3=S, I or R, Xaa4=Y or F, Xaa5=P, N, S or T, Xaa6=P, N, S or T, Xaa7=L, I or R, Xaa8=P, N, S or T.

In the following heavy chain only sequence consensus/motifs, the CDR sequences are also based on the IMGT numbering scheme.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 499, 500 and 501, respectively.

SEQ ID NO: 499: GGTFSS Xaa1 G, wherein Xaa1=Y, N or D,

SEQ ID NO: 500: IIPIFGTA, and,

SEQ ID NO: 501: Xaa1 Xaa2 Xaa3 Xaa4 Xaa5 Xaa6 S Xaa7 Xaa8, wherein Xaa1=S, E or A, Xaa2=S, R or I, Xaa3=Y, A or any acidic residue (D/E), Xaa4=Y, S, F or D, Xaa5=S, C or any aromatic residue (F/Y/W), Xaa6=Y, A or any acidic residue (D/E), Xaa7=C, V or G, and Xaa8=Y or D.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 502, 503 and 504, respectively.

SEQ ID NO: 502: Xaa1 Xaa2 Xaa3 Xaa4 Xaa5 Xaa6 Y Xaa7, wherein Xaa1=C, V or G, Xaa2=Y or S, Xaa3=P or T, Xaa4=Y, F, L or I, Xaa5=N or T, Xaa6=H, N or K, and Xaa7=Y or S,

SEQ ID NO: 503: INPYTGSA, and,

SEQ ID NO: 504: Xaa1 Xaa2 Xaa3 Xaa4 Xaa5 Xaa6 Xaa7 Xaa8 Xaa9 Xaa10 Xaa1 1 Xaa12 Xaa13 Xaa14, wherein Xaa1=S, E or A, Xaa2=S, R, K or G, Xaa3=H, N, A or any acidic residue (D/E), Xaa4=S, D, T or A, Xaa5=P, K or T, Xaa6=E, R, V or G, Xaa7=S, H, R or L, Xaa8=H, N, P, L or Q, Xaa9=Y, S or D, Xaa10=H, N, K, I or T, Xaa1 1=S, G, V, C or A, Xaa12=M, R, L or I, Xaa13=H, N, G, V, Y, A or any acidic residue (D/E), and Xaa14=I, V, F, L or A.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 505, 506 and 507, respectively.

SEQ ID NO: 505: GFT Xaa1 NSFA, wherein Xaa1=C, F or V,

SEQ ID NO: 506: ISGSGGGT, and,

SEQ ID NO: 507: Xaa1 Xaa2 D Xaa3 SP Xaa4 Xaa5 Xaa6 Xaa7 SGA Xaa8 D Xaa9, wherein Xaa1=E or A, Xaa2=N, K, T or Q, Xaa3=S, R or L, Xaa4=Y, S or D, Xaa5=Y or any acidic residue (D/E), Xaa6=F or L, Xaa7=W, L or G, Xaa8=F, L or I, and Xaa9=Y, S or D.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 508, 509 and 510, respectively.

SEQ ID NO: 508: Xaa1 FTF Xaa2 Xaa3 Xaa4 Xaa5, wherein Xaa1=C or G, Xaa2=N, S or R, Xaa3=S, N or D, Xaa4=Y, S or F, and Xaa5=S or A,

SEQ ID NO: 509: I Xaa1 GSGG Xaa2 T, wherein Xaa1=S or T, and, Xaa2=S, N, T or G, and,

SEQ ID NO: 510: Xaa1 Xaa2 R Xaa3 Xaa4 Xaa5 F Xaa6 Xaa7 Xaa8 Xaa9 D Xaa10 Xaa1 1 Xaa12 Xaa13, wherein Xaa1=E or A, Xaa2=C or G, Xaa3=P or Q, Xaa4=Y or D, Xaa5=Y or any acidic residue (D/E), Xaa6=W, L or G, Xaa7=S, R or I, Xaa8=C, V or G, Xaa9=W, C or G, Xaa10=W, C or G, Xaa1 1=F, L or V, Xaa12=A or any acidic residue (D/E), and Xaa13=H, P or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 511, 512 and 513, respectively.

SEQ ID NO: 511: Xaa1 Xaa2 TF Xaa3 Xaa4 Xaa5 Xaa6, wherein Xaa1=V or G, Xaa2=Y, F or L, Xaa3=N, S or R, Xaa4=S, N or D, Xaa5=Y, S or F, and Xaa6=S, D or A,

SEQ ID NO: 512: I Xaa1 GS Xaa2 G Xaa3 T, wherein Xaa1=S or T, Xaa2=S or G, and Xaa3=S, N, T or G, and,

SEQ ID NO: 513: Xaa1 Xaa2 Xaa3 Xaa4 Xaa5 Xaa6 Xaa7 Xaa8 Xaa9 Xaa10 GM Xaa1 1 Xaa12, wherein Xaa1=S, E or A, Xaa2=R, K or T, Xaa3=N or any acidic residue (D/E), Xaa4=D, T or A, Xaa5=K or T, Xaa6=E, R or G, Xaa7=H, R or L, Xaa8=H or P, Xaa9=Y or D, Xaa10=S, N, K, I, Y or T, Xaa1 1=Y, V or any acidic residue (D/E), and Xaa12=G, I or V.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 514, 515 and 516, respectively.

SEQ ID NO: 514: GYTL Xaa1 Xaa2 LS, wherein Xaa1=S or T, and Xaa2=any acidic residue (D/E),

SEQ ID NO: 515: FDP Xaa1 Xaa2 Xaa3 E Xaa4, wherein Xaa1=E or Q, Xaa2=any acidic residue (D/E), Xaa3=N or G, and Xaa4=I or T, and,

SEQ ID NO: 516: A Xaa1 Xaa2 Xaa3 Xaa4 Y Xaa5 Xaa6 Xaa7 Xaa8 Xaa9 Xaa10 Y Xaa1 1 G Xaa12 Xaa13 Xaa14 Xaa15 Xaa16 DV, wherein Xaa1=N, K or T, Xaa2=Y or D, Xaa3=L or V, Xaa4=W, V or G, Xaa5=Y, S or D, Xaa6=Y, S or D, Xaa7=Y or any acidic residue (D/E), Xaa8=S, R or I, Xaa9=S or R, Xaa10=V or G, Xaa1 1=Y, S or D, Xaa12=R or L, Xaa13=N or T, Xaa14=Y, S or D, Xaa15=V or G, and Xaa16=M or I.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 517, 518 and 519, respectively.

SEQ ID NO: 517: GYT Xaa1 T Xaa2 Y Xaa3, wherein Xaa1=F or L, Xaa2=S, R or N, and Xaa3=S or G,

SEQ ID NO: 518: Xaa1 S Xaa2 Xaa3 Xaa4 G Xaa5 T, wherein Xaa1=I or V, Xaa2=T, F, V or A, Xaa3=Y or N, Xaa4=S or N, and Xaa5=N or D, and,

SEQ ID NO: 519: Xaa1 K Xaa2 Xaa3 Xaa4 Xaa5 Xaa6 Xaa7 Xaa8 Xaa9 Xaa10 Xaa1 1 Xaa12 Xaa13 Xaa14 Xaa15 Xaa16 Xaa17 Xaa18 Xaa19 D Xaa20, wherein Xaa1=E or A, Xaa2=Y or D, Xaa3=F, L or V, Xaa4=V or G, Xaa5=Y or D, Xaa6=Y or D, Xaa7=Y, S or D, Xaa8=any acidic residue (D/E), Xaa9=S or R, Xaa10=S, R or N, Xaa11=V or G, Xaa12=Y or D, Xaa13=Y, S or D, Xaa14=R or G, Xaa15=R or L, Xaa16=N or T, Xaa17=Y or D, Xaa18=S, C or G, Xaa19=M, L or I, and Xaa20=F, I or V.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 520, 521 and 522, respectively.

SEQ ID NO: 520: Xaa1 Xaa2 T Xaa3 Xaa4 D Y Xaa5, wherein Xaa1=R or G, Xaa2=F or L, Xaa3=C, F or V, Xaa4=N or D, and Xaa5=S or A,

SEQ ID NO: 521: I Xaa1 WNSG Xaa2 I, wherein Xaa1=S or T, and Xaa2=S, H or R, and,

SEQ ID NO: 522: Xaa1 Xaa2 Xaa3 Xaa4 Xaa5 Xaa6 F Xaa7 Xaa8 Xaa9 Xaa10 Xaa1 1 Xaa12 Xaa13 D Xaa14, wherein Xaa15=E or A, Xaa16=C or G, Xaa17=R or L, Xaa18=P or Q, Xaa19=Y or D, Xaa20=any acidic residue (D/E), Xaa21=W or G, Xaa22=S or R, Xaa23=C or G, Xaa24=G, L, C or any aromatic residue (F/Y/W), Xaa25=H or any acidic residue (D/E), Xaa26=W or G, Xaa27=C, F or V, and Xaa28=L or P.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 523, 524 and 525, respectively.

SEQ ID NO: 523: RFTFDDY Xaa1, wherein Xaa1=S or A,

SEQ ID NO: 524: ISWNSGRI, and,

SEQ ID NO: 525: ARYG Xaa1 P Xaa2 Xaa3 Xaa4 D Xaa5, wherein Xaa1=Y or D, Xaa2=C, F or V, Xaa3=Y, S or D, Xaa4=C, F or L, and Xaa5=Y, S or D.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 526, 527 and 528, respectively.

SEQ ID NO: 526: Xaa1 Xaa2 Xaa3 F Xaa4 NY Xaa5, wherein Xaa1=V or G, Xaa2=Y or S, Xaa3=Y or S, Xaa4=S or R, and Xaa5=W, C or L,

SEQ ID NO: 527: IDPSNSYT, and,

SEQ ID NO: 528: A Xaa1 Xaa2 Xaa3 Xaa4 Xaa5 Xaa6 Xaa7 Xaa8 Xaa9 Xaa10 Xaa1 1 D Xaa12, wherein Xaa1=S or R, Xaa2=A or any acidic residue (D/E), Xaa3=R, L, I or A, Xaa4=K, T or A, Xaa5=A, T or G, Xaa6=S, C, R or G, Xaa7=R, H or N, Xaa8=Y, S or D, Xaa9=N, K, or absent, Y or T, Xaa10=C or G, Xaa1 1=M or R, and Xaa12=F, V or G.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 529, 530 and 531, respectively.

SEQ ID NO: 529: GFTF Xaa1 Xaa2 Xaa3 Xaa4, wherein Xaa1=S or N, Xaa2=S or N, Xaa3=Y or F, and Xaa4=S or A,

SEQ ID NO: 530: I Xaa1 Xaa2 S Xaa3 Xaa4 Xaa5 T, wherein Xaa1=S, N or T, Xaa2=A or G, Xaa3=S or G, Xaa4=T or G, and Xaa5=S, R, T or G, and,

SEQ ID NO: 531: A Xaa1 DLGY Xaa2 Xaa3 Xaa4 Xaa5 Xaa6 GY Xaa7 Xaa8 Xaa9 Xaa10 Xaa1 1 G Xaa12 Xaa13 V, wherein Xaa1=K or T, Xaa2=Y or D, Xaa3=Y or D, Xaa4=any acidic residue (D/E), Xaa5=S, R or I, Xaa6=S or R, Xaa7=Y or S, Xaa8=E, R or G, Xaa9=H or R, Xaa10=N, K or T, Xaa1 1=Y, S or D, Xaa12=M or I, and Xaa13=N or D.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 532, 533 and 534, respectively.

SEQ ID NO: 532: GFTF Xaa1 Xaa2 Xaa3 Xaa4, wherein Xaa1=N, S or R, Xaa2=S or D, Xaa3=Y or F, and Xaa4=S or A,

SEQ ID NO: 533: I Xaa1 Xaa2 S Xaa3 Xaa4 Xaa5 T, wherein Xaa1=S, N or T, Xaa2=A or G, Xaa3=S or G, Xaa4=T or G, and Xaa5=S, N, G, R or T, and,

SEQ ID NO: 534: A Xaa1 RG Xaa2 Y Xaa3 Xaa4 S Xaa5 Xaa6 Xaa7 YR Xaa8 Xaa9 R Xaa10 Xaa1 1 Xaa12 Xaa13 Xaa14, wherein Xaa1=S or R, Xaa2=any acidic residue (D/E), Xaa3=Y, S or D, Xaa4=S, T or A, Xaa5=E, V or G, Xaa6=S or R, Xaa7=Y or S, Xaa8=H or P, Xaa9=H or R, Xaa10=Y or D, Xaa11=C, D or G, Xaa12=M or L, Xaa13=N or D, and Xaa14=I or V.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 535, 536 and 537, respectively.

SEQ ID NO: 535: G Xaa1 Xaa2 FTRYG, wherein Xaa1=Y or S, & Xaa2=N or T,

SEQ ID NO: 536: ISTYSGNT, and,

SEQ ID NO: 537: Xaa1 R Xaa2 Xaa3 Xaa4 Xaa5 Xaa6 Xaa7 Xaa8 Xaa9 Xaa10 Xaa1 1 Xaa12 Xaa13, wherein Xaa1=S or A, Xaa2=A, S or any acidic residue (D/E), Xaa3=R, L, I or A, Xaa4=S, T or A, Xaa5=S, A, T or G, Xaa6=G, R, V, D or C, Xaa7=Y, H, R or Q, Xaa8=Y, S or D, Xaa9=Y, N or absent, Xaa10=C, V or G, Xaa1 1=M or I, Xaa12=any acidic residue (D/E), and Xaa13=I or V.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 538, 539 and 540, respectively.

SEQ ID NO: 538: G Xaa1 TFSTYG, wherein Xaa1=F or V,

SEQ ID NO: 539: IWDDGSYK, and,

SEQ ID NO: 540: A Xaa1 Xaa2 Xaa3 Xaa4 Xaa5 Xaa6 Xaa7 Xaa8 Xaa9 Xaa10 Xaa1 1 Xaa12 D Xaa13, wherein Xaa1=S, R or I, Xaa2=S or A, Xaa3=M or R, Xaa4=Y or S, Xaa5=P or T, Xaa6=M, R, L or I, Xaa7=S, D or A, Xaa8=R or L, Xaa9=R, L or I, Xaa10=W, V or G, Xaa11=W, C, L or G, Xaa12=F, L or V, and Xaa13=H, P or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 541, 542 and 543, respectively.

SEQ ID NO: 541: GFTF Xaa1 Xaa2 Xaa3 A, wherein Xaa1=N, S or R, Xaa2=S or D, and Xaa3=Y or F,

SEQ ID NO: 542: I Xaa1 Xaa2 SG Xaa3 Xaa4 T, wherein Xaa1=S, N or T, Xaa2=A or G, Xaa3=T or G, and Xaa4=S, R, N or G, and,

SEQ ID NO: 543: ARDS Xaa1 VAS Xaa2 GRG Xaa3 V Xaa4 H Xaa5 Xaa6 GM Xaa7 V, wherein Xaa1=H, N or T, Xaa2=T, K or Q, Xaa3=V or G, Xaa4=Y or D, Xaa5=Y, S or D, Xaa6=H or P, and Xaa7=N or D.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 544, 545 and 546, respectively.

SEQ ID NO: 544: Xaa1 FTF Xaa2 Xaa3 Y Xaa4, wherein Xaa1=R or G, Xaa2=N or D, Xaa3=Y or D, and Xaa4=S or A,

SEQ ID NO: 545: ISWNSG Xaa1 I, wherein Xaa1=S or R, and,

SEQ ID NO: 546: A Xaa1 Xaa2 R Xaa3 Xaa4 D Xaa5, wherein Xaa1=R or L, Xaa2=S, V or G, Xaa3=T, H, N or Q, Xaa4=R, L or V, and Xaa5=S, K, Y, Q, T or A.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 547, 548 and 549, respectively.

SEQ ID NO: 547: GYTFTNYY,

SEQ ID NO: 548: INPYTGSA, and,

SEQ ID NO: 549: ARDP Xaa1 G Xaa2 VNH Xaa3 Y Xaa4 Xaa5 D Xaa6, wherein Xaa1=C, F, L or V, Xaa2=V or G, Xaa3=F or L, Xaa4=Y, S or D, Xaa5=M, R, L or I, and Xaa6=V or G.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 550, 551 and 552, respectively.

SEQ ID NO: 550: GGSFSGYY,

SEQ ID NO: 551: INHSGST, and,

SEQ ID NO: 552: Xaa1 Xaa2 P Xaa3 Xaa4 Xaa5 Xaa6 Xaa7 ES Xaa8 Xaa9 Xaa10 Xaa1 1 Xaa12 D Xaa13, wherein Xaa1=E or A, Xaa2=M or R, Xaa3=Y, S or D, Xaa4=H, N or T, Xaa5=S or R, Xaa6=S or A, Xaa7=W, C or L, Xaa8=Y, S or D, Xaa9=Y, S or D, Xaa10=Y, S or D, Xaa11=V or G, Xaa12=M, R or L, and Xaa13=F or V.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 553, 554 and 555, respectively.

SEQ ID NO: 543: GYTFTNYY,

SEQ ID NO: 554: INPYTGSA, and,

SEQ ID NO: 555: AR Xaa1 Xaa2 Xaa3 Xaa4 Xaa5 Xaa6 Xaa7 Xaa8 Xaa9 Xaa10 Xaa1 1, wherein Xaa1=S, F, V or A, Xaa2=R, L or I, Xaa3=G or A, Xaa4=S, T or A, Xaa5=C, I or G, Xaa6=R or L, Xaa7=Y, S or D, Xaa8=C, D, V or G, Xaa9=M, R or I, Xaa10=N or D, and Xaa11=I or V.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 556, 557 and 558, respectively.

SEQ ID NO: 556: GFT Xaa1 NSFA, wherein Xaa1=F or V,

SEQ ID NO: 557: ISGSGGGT, and,

SEQ ID NO: 558: A Xaa1 Xaa2 Xaa3 Xaa4 Xaa5 Xaa6 Xaa7, wherein Xaa1=R or L, Xaa2=S, W or G, Xaa3=R or L, Xaa4=T, H, N or Q, Xaa5=G, R, I, V or L, Xaa6=any acidic residue (D/E), and Xaa7=S, K or T.

In some embodiments, the anti-IGSF8 antibody of the invention includes a monoclonal antibody or an antigen-binding portion/fragment thereof specific for IGSF8 (e.g., specific for the Ig-V set domain or the D1 domain of the ECD of IGSF8), wherein said monoclonal antibody comprises a heavy chain variable region (HCVR) comprising the HCVR CDR1, CDR2, and CDR3 of SEQ ID NOs: 559, 560 and 561, respectively.

SEQ ID NO: 559: G Xaa1 TFTRY Xaa2, wherein Xaa1=Y or S, & Xaa2=C or G,

SEQ ID NO: 560: ISTYSGNT, and,

SEQ ID NO: 561: A Xaa1 G Xaa2 Xaa3 P Xaa4 R Xaa5 H Xaa6 Xaa7 Xaa8 Xaa9 Xaa10, wherein Xaa1=R or K, Xaa2=W, V or G, Xaa3=R or L, Xaa4=Y, S or D, Xaa5=W, V or G, Xaa6=Y or D, Xaa7=C, D or G, Xaa8=M or I, Xaa9=N or any acidic residue (D/E), and Xaa10=F, I or V.

In some embodiments, the invention provides an anti-IGSF8 monoclonal antibody or an antigen-binding fragment thereof specific for IGSF8, wherein the monoclonal antibody comprises: (1) a heavy chain variable region (HCVR), comprising HCVR CDR1-CDR3 sequences at least 95% (e.g., 100%) identical to, or having up to 1, 2, 3, 4, 5, 6, 7, 8, or 9 substitutions in HCVR CDR1-CDR3, respectively, of any one of antibodies C1-C39, such as C30-C39; and, (2) a light chain variable region (LCVR), comprising LCVR CDR1-CDR3 sequences at least 95% (e.g., 100%) identical to, or having up to 1, 2, 3, 4, 5, 6, 7, 8, or 9 substitutions in LCVR CDR1-CDR3, respectively, of said any one of antibodies C1-C39, such as C30-C39. In certain embodiment, the anti-IGSF8 monoclonal antibody or an antigen-binding fragment thereof has HCVR CDR1-CDR3 and LCVR CDR1-CDR3 of one of the antibodies C1-C39, such as any one of C30-C39.

In some embodiments, the monoclonal antibody or antigen-binding fragment thereof comprises: (a) the HCVR sequence of at least 95% (e.g., 100%) identical to the HCVR sequence of any one of antibodies C1-C39, such as C30-C39; and/or, (b) the LCVR sequence of at least 95% (e.g., 100%) identical to the LCVR sequence of any one of antibodies C1-C39, such as C30-C39. In certain embodiment, the anti-IGSF8 monoclonal antibody or an antigen-binding fragment thereof has HCVR and LCVR of one of the antibodies C1-C39, such as any one of C30-C39.

In some embodiments, the invention provides an anti-IGSF8 monoclonal antibody or an antigen-binding fragment thereof specific for IGSF8, wherein the monoclonal antibody comprises: (1) a heavy chain variable region (HCVR), comprising HCVR CDR1-CDR3 sequences having up to 1, 2, or 3 residue substitutions compared to HCVR CDR1-CDR3, respectively, of any one of antibodies C1-C39, such as C30-C39; and, (2) a light chain variable region (LCVR), comprising LCVR CDR1-CDR3 sequences having up to 1, 2, or 3 residue substitutions compared to LCVR CDR1-CDR3, respectively, of said any one of antibodies C1-C39, such as C30-C39. In some embodiments, where a CDR only has 5, 4, or 3 residues, no substitution other than conserved substitute is permission (e.g., having up to 1 or 2 conservative substitutions in CDRs with no more than 5, 4, or 3 residues).

### High Affinity IGSF8 Antibody Based on Exhaustive CDR Region Mutagenesis Analysis

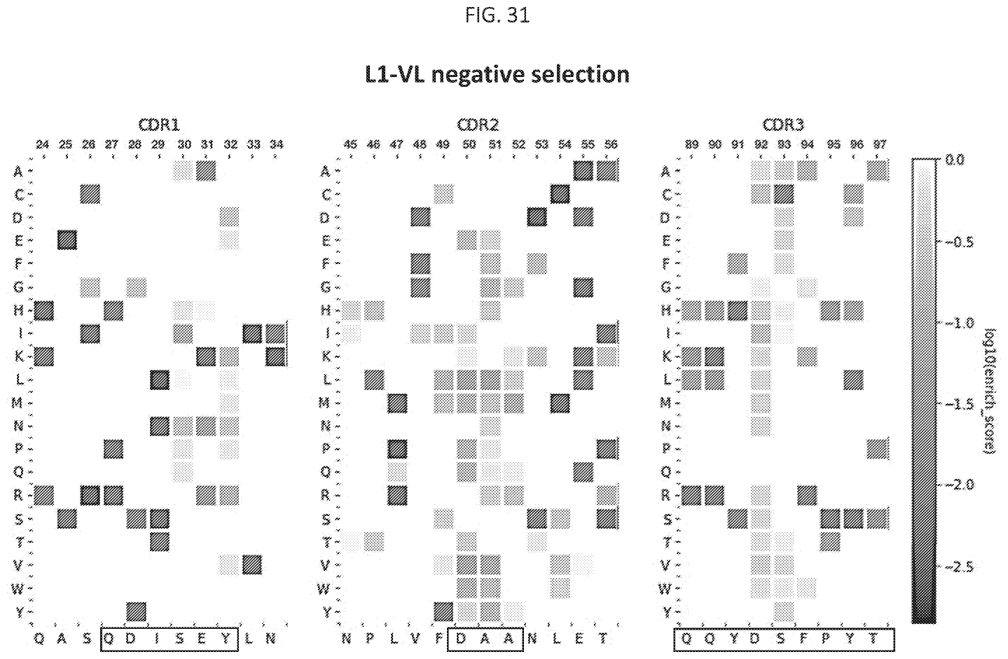

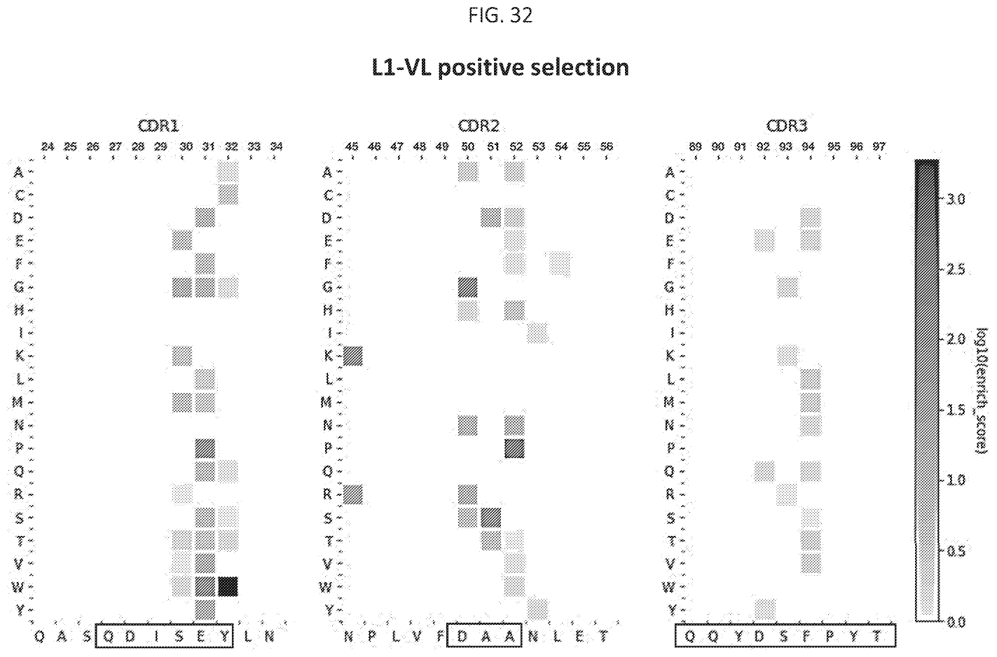

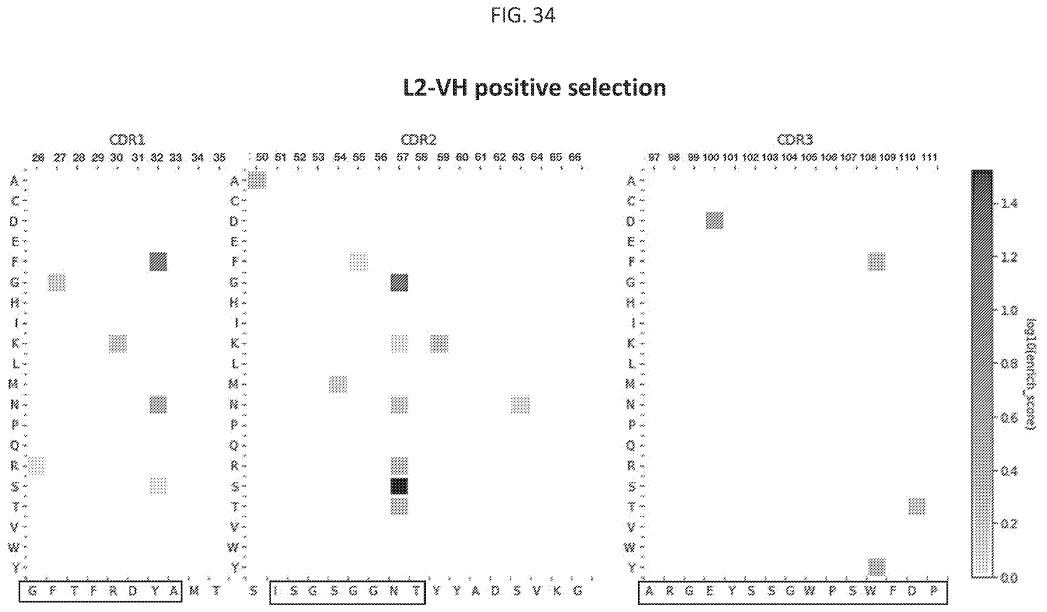

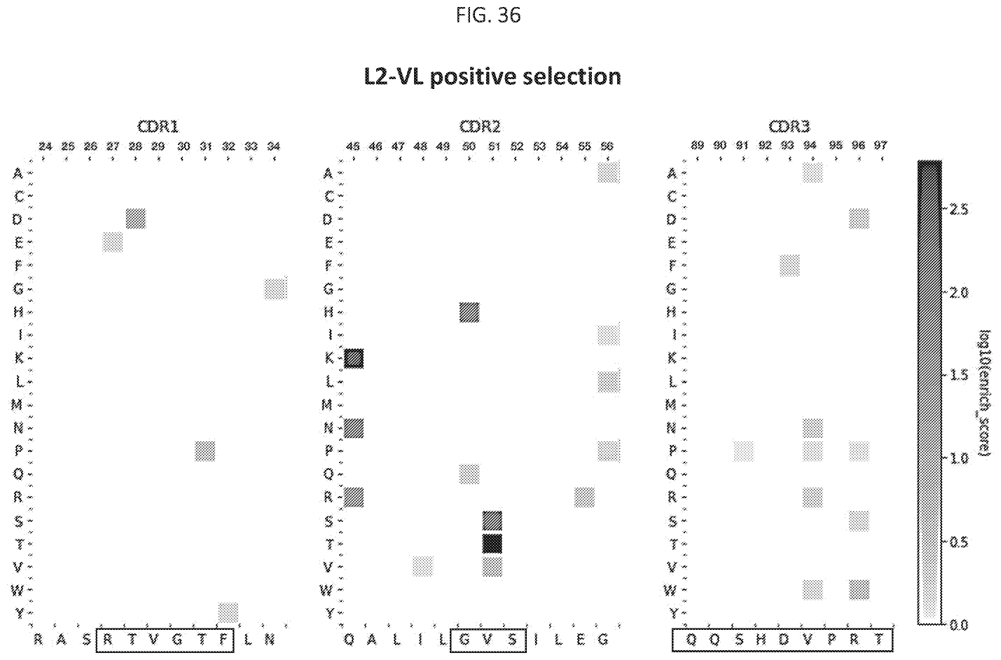

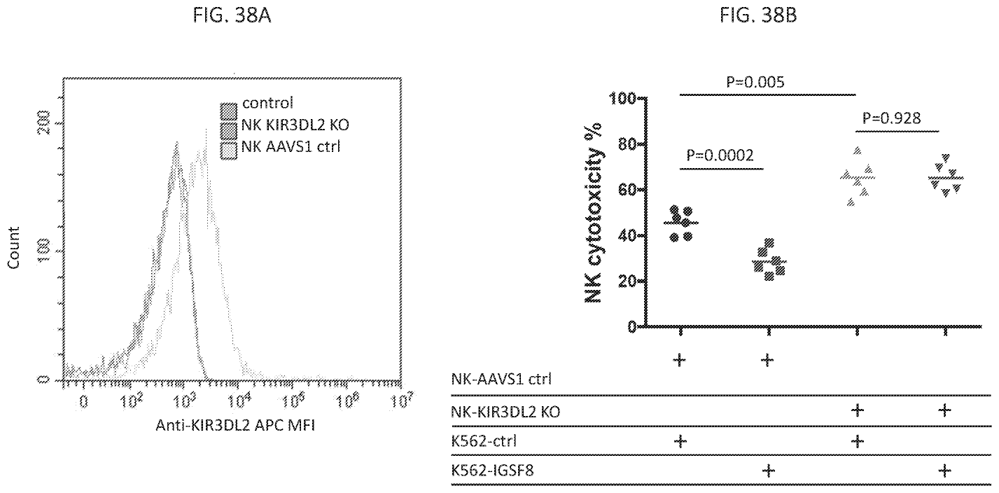

In order to identify the key residues important (or not as important) for IGSF8 binding, two specific high affinity antibodies were chosen for further CDR region sequence analysis in order to determine the relative importance of each CDR region residues, as well as framework region residues surrounding the CDR regions. Specifically, each existing residues in the two lead antibodies were replaced with 19 other amino acids individually to generate all possible mutants to assess the impact of such substitutions, and the results of each substitution are collectively represented in FIGS. 29-36. Based on this study, consensus sequences representing all acceptable substitutions (e.g., those that do not substantially affect antigen binding) as well as preferred substitutions (e.g., those that increase antigen binding compared to the original sequence, are constructed, and presented herein.

Thus the present disclosure includes amino acid consensus sequences for CDR region sequences (and in some instances, surrounding framework region sequences, based on the IMGT numbering scheme), showing specific amino acids that may be modified substituted (shown using variable “X” or “Xaa”) in antibody amino acid sequences, e.g. as described in Tables A1 and A2. Unless explicitly indicated, all antibody and CDR sequences are annotated by the IMGT numbering scheme.

Related CDR sequences that can appear in the same VH and/or VL sequences of an antibody are grouped together in the same row. For example, an antibody of the invention may comprise one each of VH CDR1, VH CDR2, VH CDR3, VL CDR1, VL CDR2, and VL CDR3, wherein said VH CDR1-VH CDR3 and VL CDR1-VL CDR3 are represented by SEQ ID NOs: 714, 715, 716, 717, 718 and 719, respectively.

Furthermore, amino acid at each Xi position (where i=1, 2, 3, . . . ) may be a selected subset of amino acids as specified in each consensus sequence. It is contemplated that any one or more of the enumerated specific amino acids at each Xi positions can be a permissible value for the Xi position. For example, in SEQ ID NO: 714, X2 may be any residues, such as A, C, D, E, F, G, H, K, M, N, P, Q, R, T, or W. In some embodiments, X2 is A or C, F or G; M, N, or Q, etc.

Unless explicitly indicated, all antibody and CDR sequences are annotated by the IMGT numbering scheme.

TABLE A1
CDR and FR consensus sequences - for CDR and adjacent FR substitutions that do not diminish binding

| VH CDR1 | VH CDR2 | VH CDR3 | VL CDR1 | VL CDR2 | VL CDR3 |
|---|---|---|---|---|---|
| X1-X2-X3-X4-X5- | X3-X4-X5-X6-X7- | X1-X2-X3-X4-X5- | X4-X5-X6-X7-X8- | X6-X7-X8 (SEQ ID | X1-X2-X3-X4-X5- |
| X6-X7-X8 (SEQ ID | X8-X9-X10 (SEQ ID | X6-X7-X8-X9-X10- | X9 (SEQ ID NO: | NO: 718) | X6-X7-X8-X9 (SEQ |
| NO: 714) | NO: 715) | X11-X12-X13 (SEQ | 717) | X6 = A, C, D, F, G, | ID NO: 719) |
| X1 = A, E, F, G, H, I, | X3 = A, C, D, E, G, | ID NO: 716) | X4 = A, C, D, E, F, | H, N, R or S, e.g., A, | X1 = A, C, D, E, F, |
| K, L, M, N, P, Q, R, | H, I, K, L, M, P, Q, | X1 = A, C, D, F, G, | G, I, K, L, M, N, Q, | G, H, N, R or S, most | G, I, M, N, P, Q, S, |
| T, V, W or Y | R, W or Y | H, I, K, L, M, N, Q, | S, T, V, W or Y | preferably G | T, V, W or Y |
| X2 = A, C, D, E, F, | X4 = A, D, E, F, H, I, | R, W or Y | X5 = A, C, D, E, F, | X7 = A, C, D, I, K, S | X2 = A, C, D, E, F, |
| G, H, K, M, N, P, Q, | K, M, N, P, Q, R, T, | X2 = A, C, D, E, F, | H, I, K, L, M, N, P, | or T, e.g., D, S, or T, | G, I, M, N, P, Q, S, |
| R, T or W | V, W or Y, e.g., R | H, L, M, N, P, Q, R, | Q, R, T, V or W | most preferably S | T, V, W or Y |
| X3 = A, C, D, E, F, | X5 = C or D | V, W or Y | X6 = A, C, D, E, F, | X8 = A, C, D, E, F, | X3 = A, C, D, E, G, |
| G, H, K, L, M, P, Q, | X6 = A, D, E, F or G, | X3 = C, D, F, I or Q | G, H, I, K, M, P, Q, | H, I, N, P, S, T, V or | I, K, L, M, N, P, Q, |
| R, T, V, W or Y | e.g., G, E, or A, most | X4 = E, F, G, H, I, K, | R, V, W or Y | W, e.g., A, D, E, F, | R, T, V, W or Y |
| X4 = A, C, D, E, F, | preferably G | L, M, N, P or Q | X7 = C, D, E, F, G, | H, N, P, T, V, or W, | X4 = D, E, F, P, Q or |
| G, H, K, M, N, P, Q, | X7 = D, E, F, G, H, I, | X5 = A, D, E, F, H, I, | K, L, M, R, S, T, V, | most preferably P | Y, e.g., E, Q, or Y |
| R, T or W | K, L, M, N, P, Q, T, | K, L, M, P, Q, S, T, | W or Y, e.g., E, G, K, | | X5 = G, K, L, M, N, |
| X5 = A, C, D, E, G, | W or Y | V, W or Y | M, T, V, or W | | P, Q, R or S, e.g., G, |
| H, I, K, L, M, N, Q, | X8 = C, F, H, K, P, | X6 = A, E, F, G, H, I, | X8 = C, D, E, F, G, | | R or K |
| R, S, V or W | R, S, T, W or Y, e.g., | K, L, M, N, P, Q, R, | H, I, L, M, P, Q, S, T, | | X6 = C, D, E, F, H, I, |
| X6 = C, D, E, F, G, | K or R, most | T, W or Y | V, W or Y, e.g., D, F, | | L, M, N, P, Q, S, T, |
| H, I, L, N, P, Q, T, V, | preferably K | X7 = A, D, E, F, H, I, | G, L, M, P, Q, S, T, | | V or Y, e.g., D, E, L, |
| W or Y | X9 = A, D, E, F, G, I, | M, N, P, Q, S, T, V, | V, W, or Y | | M, N, Q, S, T, or V |
| X7 = A, D, E, F, G, I, | K, L, M, P, Q, R, T, | W or Y, e.g., Y | X9 = A, C, F, G, H, I, | | X7 = A, C, D, E, F, |
| K, L, M, P, Q, R, S, | V, W or Y | X8 = A, C, D, F, G, | Q, S, T, W or Y, e.g., | | G, I, K, L, M, N, P, |
| T, V, W or Y | X10 = A, C, D, F, G, | H, I, K, L, M, N, P, | A, C, G, Q, S, T, or | | Q, R, V, W or Y |
| X8 = E, F, G, H, I, K, | H, I, K, L, P, Q, S, V, | Q, S, T, W or Y | W, most preferably | | X8 = A, E, F, G, I, K, |
| L, M, N, P, Q, R, T, | W or Y | X9 = A, E, G, I, K, L, | W | | M, N, P, Q, R, T, V, |
| W or Y | | M, P, Q, R, T, V, W | | | W or Y |
| | | or Y | | | X9 = C, D, E, F, G, |
| | | X10 = A, C, E, F, H, | | | H, I, K, L, M, N, Q, |
| | | I, K, L, M, N, Q or R | | | R, T, V, W or Y |
| | | X11 = D, F, G, H, M, | | | |
| | | N, P, R, T or W | | | |
| | | X12 = C, D, F, K, L, | | | |
| | | M, P, Q, R or W, | | | |
| | | e.g., R or K | | | |
| | | X13 = G, H, I, K, M, | | | |
| | | P, Q, R, W or Y | | | |
| VH CDR1 + | VH CDR2 + | VH CDR3 + | VL CDR1 + | VL CDR2 + | VL CDR3 + |
| adjacent residues | adjacent residues | adjacent residues | adjacent residues | adjacent residues | adjacent residues |
| X1-X2-X3-X4-X5- | X2-X3-X4-X5-X6- | X1-X2-X3-X4-X5- | X1-X2-X3-X4-X5- | X1-X2-X3-X4-X5- | X1-X2-X3-X4-X5- |
| X6-X7-X8-X9-X10 | X7-X8-X9-X10-X11- | X6-X7-X8-X9-X10- | X6-X7-X8-X9-X10- | X6-X7-X8-X9-X10- | X6-X7-X8-X9 (SEQ |
| (SEQ ID NO: 734) | X12-X13-X14-X15- | X11-X12-X13 (SEQ | X11 (SEQ ID NO: | X11-X12 (SEQ ID | ID NO: 739) |
| X1 = A, E, F, G, H, I, | X16-X17-X18 (SEQ | ID NO: 736) | 737) | NO: 738) | X1 = A, C, D, E, F, |
| K, L, M, N, P, Q, R, | ID NO: 735) | X1 = A, C, D, F, G, | X1 = A, C, D, E, F, | X1 = A, C, D, E, F, | G, I, M, N, P, Q, S, |
| T, V, W or Y | X2 = C, D, F, H, I, K, | H, I, K, L, M, N, Q, | G, I, L, M, N, P, Q, | G, K, L, M, N, P, Q, | T, V, W or Y |
| X2 = A, C, D, E, F, | N, P, Q, R, S, T, V, | R, W or Y | S, T, V, W or Y | R, S, V, W or Y, e.g., | X2 = A, C, D, E, F, |
| G, H, K, M, N, P, Q, | W or Y | X2 = A, C, D, E, F, | X2 = A, C, D, F, G, | K or R | G, I, M, N, P, Q, S, |
| R, T or W | X3 = A, C, D, E, G, | H, L, M, N, P, Q, R, | H, I, K, L, M, N, P, | X2 = A, C, D, E, F, | T, V, W or Y |
| X3 = A, C, D, E, F, | H, I, K, L, M, P, Q, | V, W or Y | Q, R, T, V, W or Y | G, I, K, M, N, P, Q, | X3 = A, C, D, E, G, |
| G, H, K, L, M, P, Q, | R, W or Y | X3 = C, D, F, I or Q | X3 = A, D, E, F, H, | R, S, V, W or Y | I, K, L, M, N, P, Q, |
| R, T, V, W or Y | X4 = A, D, E, F, H, I, | X4 = E, F, G, H, I, K, | K, L, M, N, P, Q, S, | X3 = A, C, D, E, F, | R, T, V, W or Y |
| X4 = A, C, D, E, F, | K, M, N, P, Q, R, T, | L, M, N, P or Q | T, V, W or Y | G, H, I, K, L, N, S, T, | X4 = D, E, F, P, Q or |
| G, H, K, M, N, P, Q, | V, W or Y, e.g., R | X5 = A, D, E, F, H, I, | X4 = A, C, D, E, F, | V, W or Y | Y, e.g., E, Q, or Y |
| R, T or W | X5 = C or D | K, L, M, P, Q, S, T, | G, I, K, L, M, N, Q, | X4 = A, C, E, H, K, | X5 = G, K, L, M, N, |
| X5 = A, C, D, E, G, | X6 = A, D, E, F or G, | V, W or Y | S, T, V, W or Y | L, M, N, P, Q, R, S, | P, Q, R or S, e.g., G, |
| H, I, K, L, M, N, Q, | e.g., G, E, or A, most | X6 = A, E, F, G, H, I, | X5 = A, C, D, E, F, | T, V, W or Y | R or K |
| R, S, V or W | preferably G | K, L, M, N, P, Q, R, | H, I, K, L, M, N, P, | X5 = A, D, E, F, G, | X6 = C, D, E, F, H, I, |
| X6 = C, D, E, F, G, | X7 = D, E, F, G, H, I, | T, W or Y | Q, R, T, V or W | H, K, N, P, Q, R, T | L, M, N, P, Q, S, T, |
| H, I, L, N, P, Q, T, V, | K, L, M, N, P, Q, T, | X7 = A, D, E, F, H, I, | X6 = A, C, D, E, F, | or W | V or Y, e.g., D, E, L, |
| W or Y | W or Y | M, N, P, Q, S, T, V, | G, H, I, K, M, P, Q, | X6 = A, C, D, F, G, | M, N, Q, S, T, or V |
| X7 = A, D, E, F, G, I, | X8 = C, F, H, K, P, | W or Y, e.g., Y | R, V, W or Y | H, N, R or S, e.g., A, | X7 = A, C, D, E, F, |
| K, L, M, P, Q, R, S, | R, S, T, W or Y, e.g., | X8 = A, C, D, F, G, | X7 = C, D, E, F, G, | G, H, N, R or S, most | G, I, K, L, M, N, P, |
| T, V, W or Y | K or R, most | H, I, K, L, M, N, P, | K, L, M, R, S, T, V, | preferably G | Q, R, V, W or Y |
| X8 = E, F, G, H, I, K, | preferably K | Q, S, T, W or Y | W or Y, e.g., E, G, K, | X7 = A, C, D, I, K, S | X8 = A, E, F, G, I, K, |
| L, M, N, P, Q, R, T, | X9 = A, D, E, F, G, I, | X9 = A, E, G, I, K, L, | M, T, V, or W | or T, e.g., D, S or T, | M, N, P, Q, R, T, V, |
| W or Y | K, L, M, P, Q, R, T, | M, P, Q, R, T, V, W | X8 = C, D, E, F, G, | most preferably S | W or Y |
| X9 = A, C, D, E, F, | V, W or Y | or Y | H, I, L, M, P, Q, S, T, | X8 = A, C, D, E, F, | X9 = C, D, E, F, G, |
| G, H, M, N, P, Q, R, | X10 = A, C, D, F, G, | X10 = A, C, E, F, H, | V, W or Y, e.g., D, F, | H, I, N, P, S, T, V or | H, I, K, L, M, N, Q, |
| S, W or Y | H, I, K, L, P, Q, S, V, | I, K, L, M, N, Q or R | G, L, M, P, Q, S, T, | W, e.g., A, D, E, F, | R, T, V, W or Y |
| X10 = A, C, D, E, F, | W or Y | X11 = D, F, G, H, M, | V, W, or Y | H, N, P, T, V, or W, | |
| G, H, I, K, M, S, T, | X11 = A, G, I, L, M, | N, P, R, T or W | X9 = A, C, F, G, H, I, | most preferably P | |
| V, or W | P, R, V, W or Y | X12 = C, D, F, K, L, | Q, S, T, W or Y, e.g., | X9 = A, C, E, G, H, | |
| | X12 = A, E, G, I, K, | M, P, Q, R or W, | A, C, G, Q, S, T, or | I, L, M, N, P, Q, R, | |
| | L, M, P, Q, R, T, V, | e.g., R or K | W, most preferably | V, W or Y, e.g., I or | |
| | W or Y | X13 = G, H, I, K, M, | W | Y | |
| | X13 = E, F, G, H, I, | P, Q, R, W or Y | X10 = A, C, D, E, F, | X10 = A, D, E, F, G, | |
| | K, L, M, N, P, Q, T, | | G, H, K, L, M, N, P, | H, I, K, L, N, P, Q, | |
| | V, W or Y, e.g., V | | Q, R, S, T, W or Y | R, T or Y, e.g., F | |
| | X14 = C, D, F, I, K, | | X11 = A, C, D, E, F, | X11 = C, E, F, H, I, | |
| | L, M, P, Q, R, S, T or | | G, H, L, M, N, P, Q, | M, N, P, R, S, T, V, | |
| | W | | R, S, T, V, W or Y | W or Y | |
| | X15 = A, D, E, F, H, | | | X12 = C, D, E, F, G, | |
| | K, L, M, P, Q, S, V, | | | H, L, M, N, Q, T, V, | |
| | W or Y | | | W or Y | |
| | X16 = C, F, K, M, N, | | | | |
| | P, Q, R, S, T, V, W | | | | |
| | or Y, e.g., F | | | | |
| | X17 = A, C, D, F, G, | | | | |
| | H, K, L, M, S, V, W | | | | |
| | or Y | | | | |
| | X18 = E, F, G, H, I, | | | | |
| | K, L, M, N, P, Q, S, | | | | |
| | T, W or Y | | | | |
| VH CDR1 | VH CDR2 | VH CDR3 | VL CDR1 | VL CDR2 | VL CDR3 |
| X1-X2-X3-X4-X5- | X2-X3-X4-X5-X6- | X1-X2-X3-X4-X5- | X4-X5-X6-X7-X8- | X6-X7-X8 (SEQ ID | X1-X2-X3-X4-X5- |
| X6-X7-X8 (SEQ ID | X7-X8-X9 (SEQ ID | X6-X7-X8-X9-X10- | X9 (SEQ ID NO: | NO: 724) | X6-X7-X8-X9 (SEQ |
| NO: 720) | NO: 721) | X11-X12-X13-X14- | 723) | X6 = C, H, I, L, M, | ID NO: 725) |
| X1 = A, C, D, E, F, | X2 = A, C, D, E, G, | X15 (SEQ ID NO: | X4 = A, C, D, E, F, | N, P, Q, W or Y, e.g., | X1 = C, D, F, G, I, K, |
| G, H, I, K, L, M, N, | H, I, K, L, M, N, P, | 722) | G, I, K, M, N, R, S, | H or Q | M, N, P, Q, S, T, V, |
| P, Q, R, S, T, V, W | Q, R, S, T, V, W or Y | X1 = A, C, D, E, F, | T, V, W or Y, e.g., E | X7 = C, D, E, F, G, | W or Y |
| or Y, e.g., R | X3 = A, C, E, F, G, | G, H, I, K, L, M, N, | X5 = C, D, E, H, K, | H, I, K, L, M, N, P, | X2 = A, C, D, F, G, I, |
| X2 = A, C, D, E, F, | H, I, K, L, M, P, Q, | P, Q, R, S, T, V, W | L, M, Q, T, W or Y, | Q, R, S, T, V, W or | L, M, N, P, Q, R, S, |
| G, H, K, L, M, N, P, | R, S, V or Y | or Y | e.g., D | Y, e.g., S, T, or V, | T, V or W |
| Q, R, S, T, V or W, | X4 = C, D, E, F, G, | X2 = F, G, H, I or T | X6 = A, C, D, E, F, | more preferably S or | X3 = C, E, G, K, M, |
| e.g., G | H, I, K, L, M, N, Q, | X3 = A, C, D, F, G, | G, H, K, M, N, P, Q, | T | P, S, V or W, e.g., P |
| X3 = A, C, D, E, F, | R, S or V | H, I, K, L, M, N, P, | R, T, V, W or Y | X8 = C, D, E, G, H, | X4 = C, H, L, M, P, |
| G, H, I, K, L, M, N, | X5 = A, C, F, H, K, | Q, R, S, T, V, W or Y | X7=C, E, G, I, L, | I, K, L, M, P, Q, R, | Q, R, V or W |
| P, Q, R, T, V, W or Y | L, M, P, Q, R, S, T, | X4 = D, E, F, H, N, | M, P, Q, V, W or Y | S, W or Y | X5 = C, D, E, F, L, |
| X4 = A, C, D, E, F, | V or W, e.g., M | Q, R, S, T, V, W or | X8 = C, M, P, Q, T | | M, P, V or W, e.g., F |
| G, H, I, K, M, N, P, | X6 = A, C, E, F, G, | Y, e.g., D | or W, e.g., P | | X6 = A, C, E, G, H, |
| Q, R, S, T, V, W or Y | H, I, K, L, M, P, Q, | X5 = A, H, I, L, M, | X9 = A, C, E, F, G, I, | | K, M, N, P, Q, R, V |
| X5 = I, K, L, M, P, | R, V or W, e.g., F | N, Q or Y | K, L, M, N, P, Q, R, | | or W, e.g., A, N, P, |
| Q, V, W or Y, e.g., K | X7 = A, C, D, E, F, | X6 = A, C, D, F, G, | T, Vor Y, e.g., Y | | R, or W |
| X6 = F, G, H, I, K, L, | G, H, I, K, L, M, N, | H, K, M, N, P, Q, R, | | | X7 = A, C, D, E, G, |
| M, P, Q, T, V, W or | P, Q, R, T, V, W or Y | S, T, V or Y | | | H, I, K, M, N, P, R, |
| Y | X8 = A, C, F, G, I, K, | X7 = A, C, E, F, H, | | | S, T, V, W or Y |
| X7 = A, C, D, E, F, | L, M, N, P, Q, R, S, | K, M, N, P, Q, S, T, | | | X8 = A, C, D, E, G, |
| G, H, I, K, M, N, P, | T, V, W or Y, e.g., G, | W or Y | | | K, M, N, P, Q, R, S |
| Q, R, S, T, V, W or | N, R, S, or T, more | X8 = A, C, D, E, F, | | | or W, e.g., D, P, S, or |
| Y, e.g., F, N or S, | preferably G or S | G, H, I, K, L, M, N, | | | W |
| more preferably F | X9 = C, D, E, F, G, | P, Q, S, T, V or W | | | X9 = C, D, E, F, G, |
| X8 = A, C, D, E, F, | H, K, L, M, N, P, Q, | X9 = A, C, D, E, F, | | | H, K, L, M, Q, R, T, |
| H, K, L, M, N, P, Q, | S, T, V, W or Y | H, I, K, L, N, Q, R, | | | V, W or Y |
| R, T or V | | S, V, W or Y | | | |
| | | X10 = A, C, D, E, F, | | | |
| | | H, I, K, L, M, N, P, | | | |
| | | Q, R, S, V, W or Y | | | |
| | | X11 = A, C, E, F, H, | | | |
| | | I, K, L, M, N, P, Q, | | | |
| | | S, T, V, W or Y | | | |
| | | X12 = F, H, I, K, N, | | | |
| | | P, Q, R, V, W or Y, | | | |
| | | e.g., F or Y | | | |
| | | X13 = A, C, D, E, F, | | | |
| | | G, H, I, K, L, N, P, | | | |
| | | Q, R, S, T, V, W or Y | | | |
| | | X14 = D, F, G, H, P, | | | |
| | | Q or T, e.g., T | | | |
| | | X15 = D, E, F, G, I, | | | |
| | | K, L, N, P, Q, R, S or | | | |
| | | T | | | |
| VH CDR1 + | VH CDR2 + | VH CDR3 + | VL CDR1 + | VL CDR2 + | VL CDR3 + |
| adjacent residues | adjacent residues | adjacent residues | adjacent residues | adjacent residues | adjacent residues |
| X1-X2-X3-X4-X5- | X1-X2-X3-X4-X5- | X1-X2-X3-X4-X5- | X1-X2-X3-X4-X5- | X1-X2-X3-X4-L-X6- | X1-X2-X3-X4-X5- |
| X6-X7-X8-X9-X10 | X6-X7-X8-X9-X10- | X6-X7-X8-X9-X10- | X6-X7-X8-X9-X10- | X7-X8-I-X10-X11- | X6-X7-X8-X9 (SEQ |
| (SEQ ID NO: 740) | X11-X12-X13-X14- | X11-X12-X13-X14- | X11 (SEQ ID NO: | X12 (SEQ ID NO: | ID NO: 745) |
| X1 = A, C, D, E, F, | X15-X16-X17 (SEQ | X15 (SEQ ID NO: | 743) | 744) | X1 = C, D, F, G, I, K, |
| G, H, I, K, L, M, N, | ID NO: 741) | 742) | X1 = A, C, D, E, F, | X1 = A, C, D, E, F, | M, N, P, Q, S, T, V, |
| P, Q, R, S, T, V, W | X1 = A, C, D, E, F, | X1 = A, C, D, E, F, | G, H, I, L, M, N, P, | G, H, K, L, N, P, R, | W or Y |
| or Y, e.g., R | H, K, L, N, P, Q, R, S | G, H, I, K, L, M, N, | R, S, T, V, W or Y | S, W or Y, e.g., K, N, | X2 = A, C, D, F, G, I, |
| X2 = A, C, D, E, F, | or Y, e.g., A | P, Q, R, S, T, V, W | X2 = A, C, D, E, F, | or R, more preferably | L, M, N, P, Q, R, S, |
| G, H, K, L, M, N, P, | X2 = A, C, D, E, G, | or Y | G, H, I, K, L, M, N, | K | T, V or W |
| Q, R, S, T, V or W, | H, I, K, L, M, N, P, | X2 = F, G, H, I or T | Q, R, S, V, W or Y | X2 = A, C, D, E, G, | X3 = C, E, G, K, M, |
| e.g., G | Q, R, S, T, V, W or Y | X3 = A, C, D, F, G, | X3 = A, C, D, E, F, | H, K, M, N, Q, T or | P, S, V or W, e.g., P |
| X3 = A, C, D, E, F, | X3 = A, C, E, F, G, | H, I, K, L, M, N, P, | G, H, I, K, L, M, N, | W | X4 = C, H, L, M, P, |
| G, H, I, K, L, M, N, | H, I, K, L, M, P, Q, | Q, R, S, T, V, W or Y | Q, S, T, V, W or Y | X3 = A, C, D, E, F, | Q, R, V or W |
| P, Q, R, T, V, W or Y | R, S, V or Y | X4 = D, E, F, H, N, | X4 = A, C, D, E, F, | G, H, K, L, M, N, P, | X5 = C, D, E, F, L, |
| X4 = A, C, D, E, F, | X4 = C, D, E, F, G, | Q, R, S, T, V, W or | G, I, K, M, N, R, S, | Q, R, S, T, W or Y | M, P, V or W, e.g., F |
| G, H, I, K, M, N, P, | H, I, K, L, M, N, Q, | Y, e.g., D | T, V, W or Y, e.g., E | X4 = A, C, D, E, F, | X6 = A, C, E, G, H, |
| Q, R, S, T, V, W or Y | R, S or V | X5 = A, H, I, L, M, | X5 = C, D, E, H, K, | G, H, I, K, M, N, P, | K, M, N, P, Q, R, V |
| X5 = I, K, L, M, P, | X5 = A, C, F, H, K, | N, Q or Y | L, M, Q, T, W or Y, | Q, R, S, V, W or Y, | or W, e.g., A, N, P, |
| Q, V, W or Y, e.g., K | L, M, P, Q, R, S, T, | X6 = A, C, D, F, G, | e.g., D | e.g., V | R, or W |
| X6 = F, G, H, I, K, L, | V or W, e.g., M | H, K, M, N, P, Q, R, | X6 = A, C, D, E, F, | X6 = C, H, I, L, M, | X7 = A, C, D, E, G, |
| M, P, Q, T, V, W or | X6 = A, C, E, F, G, | S, T, V or Y | G, H, K, M, N, P, Q, | N, P, Q, W or Y, e.g., | H, I, K, M, N, P, R, |
| Y | H, I, K, L, M, P, Q, | X7 = A, C, E, F, H, | R, T, V, W or Y | H or Q | S, T, V, W or Y |
| X7 = A, C, D, E, F, | R, V or W, e.g., F | K, M, N, P, Q, S, T, | X7 = C, E, G, I, L, | X7 = C, D, E, F, G, | X8 = A, C, D, E, G, |
| G, H, I, K, M, N, P, | X7 = A, C, D, E, F, | W or Y | M, P, Q, V, W or Y | H, I, K, L, M, N, P, | K, M, N, P, Q, R, S |
| Q, R, S, T, V, W or | G, H, I, K, L, M, N, | X8 = A, C, D, E, F, | X8 = C, M, P, Q, T | Q, R, S, T, V, W or | or W, e.g., D, P, S, or |
| Y, e.g., F, N or S, | P, Q, R, T, V, W or Y | G, H, I, K, L, M, N, | or W, e.g., P | Y, e.g., S, T, or V, | W |
| more preferably F | X8 = A, C, F, G, I, K, | P, Q, S, T, V or W | X9 = A, C, E, F, G, I, | more preferably S or | X9 = C, D, E, F, G, |
| X8 = A, C, D, E, F, | L, M, N, P, Q, R, S, | X9 = A, C, D, E, F, | K, L, M, N, P, Q, R, | T | H, K, L, M, Q, R, T, |
| H, K, L, M, N, P, Q, | T, V, W or Y, e.g., G, | H, I, K, L, N, Q, R, | T, V or Y, e.g., Y | X8 = C, D, E, G, H, | V, W or Y |
| R, T or V | K, N, R, S, or T, | S, V, W or Y | X10 = A, C, D, E, F, | I, K, L, M, P, Q, R, | |
| X9 = A, C, D, E, F, | more preferably G or | X10 = A, C, D, E, F, | G, H, K, L, M, N, P, | S, W or Y | |
| G, H, K, M, N, P, Q, | S | H, I, K, L, M, N, P, | Q, R, S, T, W or Y | X10 = A, C, D, E, G, | |
| R, S, T, V, W or Y | X9 = C, D, E, F, G, | Q, R, S, V, W or Y | X11 = C, D, E, F, G, | H, I, K, L, M, N, P, | |
| X10 = A, C, D, E, F, | H, K, L, M, N, P, Q, | X11 = A, C, E, F, H, | I, L, M, N, P, Q, R, | Q, T, W or Y | |
| I, K, L, M, P, Q, R, | S, T, V, W or Y | I, K, L, M, N, P, Q, | W or Y, e.g., G | X11 = C, E, G, I, L, | |
| T, V, W or Y | X10 = A, C, E, H, K, | S, T, V, W or Y | | M, P, R, S, T, W or | |
| | L, M, P, S, T, V, W | X12 = F, H, I, K, N, | | Y, e.g., R | |
| | or Y, e.g., K | P, Q, R, V, W or Y, | | X12 = A, E, F, G, H, | |
| | X11 = A, C, D, E, F, | e.g., F or Y | | I, K, L, M, P, Q, V, | |
| | G, H, I, K, L, M, N, | X13 = A, C, D, E, F, | | W or Y, e.g., A, I, L, | |
| | P, Q, R, T, V, W or Y | G, H, I, K, L, N, P, | | or P | |
| | X12 = A, C, D, E, F, | Q, R, S, T, V, W or Y | | | |
| | H, I, K, L, M, N, P, | X14 = D, F, G, H, P, | | | |
| | Q, R, S, T, V, W or Y | Q or T, e.g., T | | | |
| | X13 = A, C, D, E, F, | X15 = D, E, F, G, I, | | | |
| | G, H, I, K, L, M, N, | K, L, N, P, Q, R, S or | | | |
| | R, S, T, V, W or Y | T | | | |
| | X14 = A, C, D, F, G, | | | | |
| | H, I, L, M, N, P, Q, | | | | |
| | R, S, T, V, W or Y, | | | | |
| | e.g., N | | | | |
| | X15 = A, C, D, E, G, | | | | |
| | H, I, K, L, M, N, P, | | | | |
| | Q, R, S, T, V, W or Y | | | | |
| | X16 = A, C, D, F, G, | | | | |
| | H, I, K, L, M, N, P, | | | | |
| | S, T, V, W or Y | | | | |
| | X17 = A, C, E, F, G, | | | | |
| | H, I, K, L, M, N, P, | | | | |
| | Q, R, S, T, V, W or Y | | | | |

In certain embodiments, in any of the Xi residue definitions in Table A1, the residues after “e.g.” have enhanced binding compared to the original residues at the same position. Antibody consensus sequences with such enhanced binding are provided in Table A2.

TABLE A2
CDR and FR consensus sequence - for CDR and FR substitutions that enhances binding

| VH CDR1 | VH CDR2 | VH CDR3 | VL CDR1 | VL CDR2 | VL CDR3 |
|---|---|---|---|---|---|
| X1-X2-X3-X4-X5-X6- | X3-X4-X5-X6-X7-X8- | X1-X2-X3-X4-X5-X6- | X4-X5-X6-X7-X8-X9 | X6-X7-X8 (SEQ ID | X1-X2-X3-X4-X5-X6- |
| X7-X8 (SEQ ID NO: | X9-X10 (SEQ ID NO: | X7-X8-X9-X10-X11- | (SEQ ID NO: 757) | NO: 758) | X7-X8-X9 (SEQ ID |
| 754) | 755) | X12-X13 (SEQ ID NO: | X4 = A, C, D, E, F, G, | X6 = A, G, H, N, R or | NO: 759) |
| X1 = A, E, F, G, H, I, | X3 = A, C, D, E, G, H, | 756) | I, K, L, M, N, Q, S, T, | S, most preferably G | X1 = A, C, D, E, F,G, |
| K, L, M, N, P, Q, R, T, | I, K, L, M, P, Q, R, W | X1 = A, C, D, F, G, H, | V, W or Y | X7 = D, S, or T, most | I, M, N, P, Q, S, T, V, |
| V, W or Y | or Y | I, K, L, M, N, Q, R, W | X5 = A, C, D, E, F, H, | preferably S | W or Y |
| X2 = A, C, D, E, F, G, | X4 = R | or Y | I, K, L, M, N, P, Q, R, | X8 = A, D, E, F, H, N, | X2 = A, C, D, E, F, G, |
| H, K, M, N, P, Q, R, T | X5 = C or D | X2 = A, C, D, E, F, H, | T, V or W | P, T, V, or W, most | I, M, N, P, Q, S, T, V, |
| or W | X6 = G, E, or A, most | L, M, N, P, Q, R, V, W | X6 = A, C, D, E, F, G, | preferably P | W or Y |
| X3 = A, C, D, E, F, G, | preferably G | or Y | H, I, K, M, P, Q, R, V, | | X3 = A, C, D, E, G, I, |
| H, K, L, M, P, Q, R, T, | X7 = D, E, F, G, H, I, | X3 = C, D, F, I or Q | W or Y | | K, L, M, N, P, Q, R, T, |
| V, W or Y | K, L, M, N, P, Q, T, W | X4 = E, F, G, H, I, K, | X7 = E, G, K, M, T, V, | | V, W or Y |
| X4 = A, C, D, E, F, G, | or Y | L, M, N, P or Q | or W | | X4 = E, Q, or Y |
| H, K, M, N, P, Q, R, T | X8 = K or R, most | X5 = A, D, E, F, H, I, | X8 = D, F, G, L, M, P, | | X5 = G, R or K |
| or W | preferably K | K, L, M, P, Q, S, T, V, | Q, S, T, V, W, or Y | | X6 = D, E, L, M, N, Q, |
| X5 = A, C, D, E, G, H, | X9 = A, D, E, F, G, I, | W or Y | X9 = A, C, G, Q, S, T, | | S, T, or V |
| I, K, L, M, N, Q, R, S, | K, L, M, P, Q, R, T, V, | X6 = A, E, F, G, H, I, | or W, most preferably | | X7 = A, C, D, E, F, G, |
| V or W | W or Y | K, L, M, N, P, Q, R, T, | W | | I, K, L, M, N, P, Q, R, |
| X6 = C, D, E, F, G, H, | X10 = A, C, D, F, G, | W or Y | | | V, W or Y |
| I, L, N, P, Q, T, V, W | H, I, K, L, P, Q, S, V, | X7 = Y | | | X8 = A, E, F, G, I, K, |
| or Y | W or Y | X8 = A, C, D, F, G, H, | | | M, N, P, Q, R, T, V, W, |
| X7 = A, D, E, F, G, I, | | I, K, L, M, N, P, Q, S, | | | or Y |
| V, W or Y | | T, W or Y | | | X9 = C, D, E, F, G, H, |
| X8 = E, F, G, H, I, K, | | X9 = A, E, G, I, K, L, | | | I, K, L, M, N, Q, R, T, |
| L, M, N, P, Q, R, T, W | | M, P, Q, R, T, V, W or | | | V, W or Y |
| or Y | | Y | | | |
| | | X10 = A, C, E, F, H, I, | | | |
| | | K, L, M, N, Q or R | | | |
| | | X11 = D, F, G, H, M, | | | |
| | | N, P, R, T or W | | | |
| | | X12 = R or K | | | |
| | | X13 = G, H, I, K, M, P, | | | |
| | | Q, R, W or Y | | | |
| VH CDR1 + adjacent | VH CDR2 + adjacent | VH CDR3 + adjacent | VL CDR1 + adjacent | VL CDR2 + adjacent | VL CDR3 + adjacent |
| residues | residues | residues | residues | residues | residues |
| X1-X2-X3-X4-X5-X6- | X1-X2-X3-X4-X5-X6- | X1-X2-X3-X4-X5-X6- | X1-X2-X3-X4-X5-X6- | X1-X2-X3-X4-X5-X6- | X1-X2-X3-X4-X5-X6- |
| X7-X8-X9-X10 (SEQ | X7-X8-X9-X10-X11- | X7-X8-X9-X10-X11- | X7-X8-X9-X10-X11 | X7-X8-X9-X10-X11- | X7-X8-X9 (SEQ ID |
| ID NO: 774) | X12-X13-X14-X15- | X12-X13 (SEQ ID NO: | (SEQ ID NO: 777) | X12 (SEQ ID NO: 778) | NO: 779) |
| X1 = A, E, F, G, H, I, | X16-X17-X18 (SEQ | 776) | X1 = A, C, D, E, F, G, | X1 = K or R | X1 = A, C, D, E, F, G, |
| K, L, M, N, P, Q, R, T, | ID NO: 775) | X1 = A, C, D, F, G, H, | I, L, M, N, P, Q, S, T, | X2 = A, C, D, E, F, G, | I, M, N, P, Q, S, T, V, |
| V, W or Y | X1 = A, C, E, F, G, H, | I, K, L, M, N, Q, R, W | V, W or Y | I, K, M, N, P, Q, R, S, | W or Y |
| X2 = A, C, D, E, F, G, | I, K, L, M, N, Q, R, W | or Y | X2 = A, C, D, F, G, H, | V, W or Y | X2 = A, C, D, E, F, G, |
| H, K, M, N, P, Q, R, T | or Y | X2 = A, C, D, E, F, H, | I, K, L, M, N, P, Q, R, | X3 = A, C, D, E, F, G, | I, M, N, P, Q, S, T, V, |
| or W | X2 = C, D, F, H, I, K, | L, M, N, P, Q, R, V, W | T, V, W or Y | H, I, K, L, N, S, T, V, | W or Y |
| X3 = A, C, D, E, F, G, | N, P, Q, R, S, T, V, W | or Y | X3 = A, D, E, F, H, K, | W or Y | X3 = A, C, D, E, G, I, |
| H, K, L, M, P, Q, R, T, | or Y | X3 = C, D, F, I or Q | L, M, N, P, Q, S, T, V, | X4 = A, C, E, H, K, L, | K, L, M, N, P, Q, R, T, |
| V, W or Y | X3 = A, C, D, E, G, H, | X4 = E, F, G, H, I, K, | W or Y | M, N, P, Q, R, S, T, V, | V, W or Y |
| X4 = A, C, D, E, F, G, | I, K, L, M, P, Q, R, W | L, M, N, P or Q | X4 = A, C, D, E, F, G, | W or Y | X4 = E, Q, or Y |
| H, K, M, N, P, Q, R, T | or Y | X5 = A, D, E, F, H, I, | I, K, L, M, N, Q, S, T, | X5 = A, D, E, F, G, H, | X5 = G, R or K |
| or W | X4 = R | K, L, M, P, Q, S, T, V, | V, W or Y | K, N, P, Q, R, T or W | X6 = D, E, L, M, N, Q, |
| X5 = A, C, D, E, G, H, | X5 = C or D | W or Y | X5 = A, C, D, E, F, H, | X6 = A, G, H, N, R or | S, T, or V |
| I, K, L, M, N, Q, R, S, | X6 = G, E, or A, most | X6 = A, E, F, G, H, I, | I, K, L, M, N, P, Q, R, | S, most preferably G | X7 = A, C, D, E, F,G, |
| V or W | preferably G | K, L, M, N, P, Q, R, T, | T, V or W | X7 = D, S or T, most | I, K, L, M, N, P, Q, R, |
| X6 = C, D, E, F, G, H, | X7 = D, E, F, G, H, I, | W or Y | X6 = A, C, D, E, F, G, | preferably S | V, W orY |
| I, L, N, P, Q, T, V, W | K, L, M, N, P, Q, T, W | X7 = Y | H, I, K, M, P, Q, R, V, | X8 = A, D, E, F, H, N, | X8 = A, E, F, G, I, K, |
| or Y | or Y | X8 = A, C, D, F, G, H, | W or Y | P, T, V, or W, most | M, N, P, Q, R, T, V, W |
| X7 = A, D, E, F, G, I, | X8 = K or R, most | I, K, L, M, N, P, Q, S, | X7 = E, G, K, M, T, V, | preferably P | or Y |
| K, L, M, P, Q, R, S, T, | preferably K | T, W or Y | or W | X9 = I or Y | X9 = C, D, E, F, G, H, |
| V, W or Y | X9 = A, D, E, F, G, I, | X9 = A, E, G, I, K, L, | X8 = D, F, G, L, M, P, | X10 = F | I, K, L, M, N, Q, R, T, |
| X8 = E, F, G, H, I, K, | K, L, M, P, Q, R, T, V, | M, P, Q, R, T, V, W or | Q, S, T, V, W, or Y | X11 = C, E, F, H, I, M, | V, W or Y |
| L, M, N, P, Q, R, T, W | W or Y | Y | X9 = A, C, G, Q, S, T, | N, P, R, S, T, V, W or | |
| or Y | X10 = A, C, D, F, G, | X10 = A, C, E, F, H, I, | or W, most preferably | Y | |
| X9 = A, C, D, E, F, G, | H, I, K, L, P, Q, S, V, | K, L, M, N, Q or R | W | X12 = C, D, E, F, G, H, | |
| H, M, N, P, Q, R, S, W | W or Y | X11 = D, F, G, H, M, | X10 = A, C, D, E, F, G, | L, M, N, Q, T, V, W or | |
| or Y | X11= A, G, I, L, M, P, | N, P, R, T or W | H, K, L, M, N, P, Q, R, | Y | |
| X10 = A, C, D, E, F, G, | R, V, W or Y | X12 = R or K | S, T, W or Y | | |
| H, I, K, M, S, T, V, or | X12 = A, E, G, I, K, L, | X13 = G, H, I, K, M, P, | X11 = A, C, D, E, F, G, | | |
| W | M, P, Q, R, T, V, W or | Q, R, W or Y | H, L, M, N, P, Q, R, S, | | |
| | Y | | T, V, W or Y | | |
| | X13 = V | | | | |
| | X14 = C, D, F, I, K, L, | | | | |
| | M, P, Q, R, S, T or W | | | | |
| | X15 = A, D, E, F, H, | | | | |
| | K, L, M, P, Q, S, V, W | | | | |
| | or Y | | | | |
| | X16 = F | | | | |
| | X17 = A, C, D, F, G, | | | | |
| | H, K, L, M, S, V, W or | | | | |
| | Y | | | | |
| | X18 = E, F, G, H, I, K, | | | | |
| | L, M, N, P, Q, S, T, W | | | | |
| | or Y | | | | |
| VH CDR1 | VH CDR2 | VH CDR3 | VL CDR1 | VL CDR2 | VL CDR3 |
| X1-X2-X3-X4-X5-X6- | X2-X3-X4-X5-X6-X7- | X1-X2-X3-X4-X5-X6- | X4-X5-X6-X7-X8-X9 | X6-X7-X8 (SEQ ID | X1-X2-X3-X4-X5-X6- |
| X7-X8 (SEQ ID NO: | X8-X9 (SEQ ID NO: | X7-X8-X9-X10-X11- | (SEQ ID NO: 763) | NO: 764) | X7-X8-X9 (SEQ ID |
| 760) | 761) | X12-X13-X14-X15 | X4 = E | X6 = H or Q | NO: 765) |
| X1 = R | X2 = A, C, D, E, G, H, | (SEQ ID NO: 762) | X5 = D | X7 = S, T, or V, more | X1 = C, D, F, G, I, K, |
| X2 = G | I, K, L, M, N, P, Q, R, | X1 = A, C, D, E, F, G, | X6 = A, C, D, E, F, G, | preferably S or T | M, N, P, Q, S, T, V, W |
| X3 = A, C, D, E, F, G, | S, T, V, W or Y | H, I, K, L, M, N, P, Q, | H, K, M, N, P, Q, R, T, | X8 = C, D, E, G, H, I, | or Y |
| H, I, K, L, M, N, P, Q, | X3 = A, C, E, F, G, H, | R, S, T, V, W or Y | V, W or Y | K, L, M, P, Q, R, S, W | X2 = A, C, D, F, G, I, |
| R, T, V, W or Y | I, K, L, M, P, Q, R, S, | X2 = F, G, H, I or T | X7 = C, E, G, I, L, M, | or Y | L, M, N, P, Q, R, S, T, |
| X4 = A, C, D, E, F, G, | V or Y | X3 = A, C, D, F, G, H, | P, Q, V, W or Y | | V or W |
| H, I, K, M, N, P, Q, R, | X4 = C, D, E, F, G, H, | I, K, L, M, N, P, Q, R, | X8 = P | | X3 = P |
| S, T, V, W or Y | I, K, L, M, N, Q, R, S | S, T, V, W or Y | X9 = Y | | X4 = C, H, L, M, P, Q, |
| X5 = K | or V | X4 = D | | | R, V or W |
| X6 = F, G, H, I, K, L, | X5 = M | X5 = A, H, I, L, M, N, | | | X5 = F |
| M, P, Q, T, V, W or Y | X6 = F | Q or Y | | | X6 = A, N, P, R, or W |
| X7 = F, N or S, more | X7 = A, C, D, E, F, G, | X6 = A, C, D, F, G, H, | | | X7 = A, C, D, E, G, H, |
| preferably F | H, I, K, L, M, N, P, Q, | K, M, N, P, Q, R, S, T, | | | I, K, M, N, P, R, S, T, |
| X8 = A, C, D, E, F, H, | R, T, V, W or Y | V or Y | | | V, W or Y |
| K, L, M, N, P, Q, R, T | X8 = G, K, N, R, S, or | X7 = A, C, E, F, H, K, | | | X8 = D, P, S, or W |
| or V | T, more preferably G or | M, N, P, Q, S, T, W or | | | X9 = C, D, E, F, G, H, |
| | S | Y | | | K, L, M, Q, R, T, V, W |
| | X9 = C, D, E, F, G, H, | X8 = A, C, D, E, F, G, | | | or Y |
| | K, L, M, N, P, Q, S, T, | H, I, K, L, M, N, P, Q, | | | |
| | V, W or Y | S, T, V or W | | | |
| | | X9 = A, C, D, E, F, H, | | | |
| | | I, K, L, N, Q, R, S, V, | | | |
| | | W or Y | | | |
| | | X10 = A, C, D, E, F, H, | | | |
| | | I, K, L, M, N, P, Q, R, | | | |
| | | S, V, W or Y | | | |
| | | X11 = A, C, E, F, H, I, | | | |
| | | K, L, M, N, P, Q, S, T, | | | |
| | | V, W or Y | | | |
| | | X12 = F or Y | | | |
| | | X13 = A, C, D, E, F, G, | | | |
| | | H, I, K, L, N, P, Q, R, | | | |
| | | S, T, V, W or Y | | | |
| | | X14 = T | | | |
| | | X15 = D, E, F, G, I, K, | | | |
| | | L, N, P, Q, R, S or T | | | |
| VH CDR1 + adjacent | VH CDR2 + adjacent | VH CDR3 + adjacent | VL CDR1 + adjacent | VL CDR2 + adjacent | VL CDR3 + adjacent |
| residues | residues | residues | residues | residues | residues |
| X1-X2-X3-X4-X5-X6- | X1-X2-X3-X4-X5-X6- | X1-X2-X3-X4-X5-X6- | X1-X2-X3-X4-X5-X6- | X1-X2-X3-X4-L-X6- | X1-X2-X3-X4-X5-X6- |
| X7-X8-X9-X10 (SEQ | X7-X8-X9-X10-X11- | X7-X8-X9-X10-X11- | X7-X8-X9-X10-X11 | X7-X8-I-X10-X11- | X7-X8-X9 (SEQ ID |
| ID NO: 780) | X12-X13-X14-X15- | X12-X13-X14-X15 | (SEQ ID NO: 783) | X12 (SEQ ID NO: 784) | NO: 785) |
| X1 = R | X16-X17 (SEQ ID NO: | (SEQ ID NO: 782) | X1 = A, C, D, E, F, G, | X1 = K, N, or R, more | X1 = C, D, F, G, I, K, |
| X2 = G | 781) | X1 = A, C, D, E, F, G, | H, I, L, M, N, P, R, S, | preferably K | M, N, P, Q, S, T, V, W |
| X3 = A, C, D, E, F, G, | X1 = A | H, I, K, L, M, N, P, Q, | T, V, W or Y | X2 = A, C, D, E, G, H, | or Y |
| H, I, K, L, M, N, P, Q, | X2 = A, C, D, E, G, H, | R, S, T, V, W or Y | X2 = A, C, D, E, F, G, | K, M, N, Q, T or W | X2 = A, C, D, F, G, I, |
| R, T, V, W or Y | I, K, L, M, N, P, Q, R, | X2 = F, G, H, I or T | H, I, K, L, M, N, Q, R, | X3 = A, C, D, E, F, G, | L, M, N, P, Q, R, S, T, |
| X4 = A, C, D, E, F, G, | S, T, V, W or Y | X3 = A, C, D, F, G, H, | S, V, W or Y | H, K, L, M, N, P, Q, R, | V or W |
| H, I, K, M, N, P, Q, R, | X3 = A, C, E, F, G, H, | I, K, L, M, N, P, Q, R, | X3 = A, C, D, E, F, G, | S, T, W or Y | X3 = P |
| S, T, V, W or Y | I, K, L, M, P, Q, R, S, | S, T, V, W or Y | H, I, K, L, M, N, Q, S, | X4 = V | X4 = C, H, L, M, P, Q, |
| X5 = K | V or Y | X4 = D | T, V, W or Y | X6 = H or Q | R, V or W |
| X6 = F, G, H, I, K, L, | X4 = C, D, E, F, G, H, | X5 = A, H, I, L, M, N, | X4 = E | X7 = S, T, or V, more | X5 = F |
| M, P, Q, T, V, W or Y | I, K, L, M, N, Q, R, S | Q or Y | X5 = D | preferably S or T | X6 = A, N, P, R, or W |
| X7 = F, N or S, more | or V | X6 = A, C, D, F, G, H, | X6 = A, C, D, E, F, G, | X8 = C, D, E, G, H, I, | X7 = A, C, D, E, G, H, |
| preferably F | X5 = M | K, M, N, P, Q, R, S, T, | H, K, M, N, P, Q, R, T, | K, L, M, P, Q, R, S, W | I, K, M, N, P, R, S, T, |
| X8 = A, C, D, E, F, H, | X6 = F | V or Y | V, W or Y | or Y | V, W or Y |
| K, L, M, N, P, Q, R, T | X7 = A, C, D, E, F, G, | X7 = A, C, E, F, H, K, | X7 = C, E, G, I, L, M, | X10 = A, C, D, E, G, | X8 = D, P, S, or W |
| or V | H, I, K, L, M, N, P, Q, | M, N, P, Q, S, T, W or | P, Q, V, W or Y | H, I, K, L, M, N, P, Q, | X9 = C, D, E, F, G, H, |
| X9 = A, C, D, E, F, G, | R, T, V, W or Y | Y | X8 = P | T, W or Y | K, L, M, Q, R, T, V, W |
| H, K, M, N, P, Q, R, S, | X8 = G, K, N, R, S, or | X8 = A, C, D, E, F, G, | X9 = Y | X11 = R | or Y |
| T, V, W or Y | T, more preferably G or | H, I, K, L, M, N, P, Q, | X10 = A, C, D, E, F, G, | X12 = A, I, L, or P | |
| X10 = A, C, D, E, F, I, | S | S, T, V or W | H, K, L, M, N, P, Q, R, | | |
| K, L, M, P, Q, R, T, V, | X9 = C, D, E, F, G, H, | X9 = A, C, D, E, F, H, | S, T, W or Y | | |
| W or Y | K, L, M, N, P, Q, S, T, | I, K, L, N, Q, R, S, V, | X11 = G | | |
| | V, W or Y | W or Y | | | |
| | X10 = K | X10 = A, C, D, E, F, H, | | | |
| | X11 = A, C, D, E, F, G, | I, K, L, M, N, P, Q, R, | | | |
| | H, I, K, L, M, N, P, Q, | S, V, W or Y | | | |
| | R, T, V, W or Y | X11 = A, C, E, F, H, I, | | | |
| | X12 = A, C, D, E, F, H, | K, L, M, N, P, Q, S, T, | | | |
| | I, K, L, M, N, P, Q, R, | V, W or Y | | | |
| | S, T, V, W or Y | X12 = F or Y | | | |
| | X13 = A, C, D, E, F, G, | X13 = A, C, D, E, F, G, | | | |
| | H, I, K, L, M, N, R, S, | H, I, K, L, N, P, Q, R, | | | |
| | T, V, W or Y | S, T, V, W or Y | | | |
| | X14 = N | X14 = T | | | |
| | X15 = A, C, D, E, G, | X15 = D, E, F, G, I, K, | | | |
| | H, I, K, L, M, N, P, Q, | L, N, P, Q, R, Sor T | | | |
| | R, S, T, V, W or Y | | | | |
| | X16 = A, C, D, F, G, | | | | |
| | H, I, K, L, M, N, P, S, | | | | |
| | T, V, W or Y | | | | |
| | X17 = A, C, E, F, G, H, | | | | |
| | I, K, L, M, N, P, Q, R, | | | | |
| | S, T, V, W or Y | | | | |

Thus, in certain embodiments, the anti-IGSF8 antibody or antigen binding fragment thereof comprises a VH CDR1, a VH CDR2, a VH CDR3, a VL CDR1, a VL CDR2, and a VL CDR3 that comprise, consist essentially of, or consist of the amino acid sequences of SEQ ID NOs: 714, 715, 716, 717, 718, and 719, respectively.

In certain embodiments, the anti-IGSF8 antibody or antigen binding fragment thereof comprises a VH CDR1, a VH CDR2, a VH CDR3, a VL CDR1, a VL CDR2, and a VL CDR3 that comprise, consist essentially of, or consist of the amino acid sequences of SEQ ID NOs: 720, 721, 722, 723, 724, and 725, respectively.

In certain embodiments, the anti-IGSF8 antibody or antigen binding fragment thereof comprises a VH CDR1, a VH CDR2, a VH CDR3, a VL CDR1, a VL CDR2, and a VL CDR3 that comprise, consist essentially of, or consist of the amino acid sequences of SEQ ID NOs: 754, 755, 756, 757, 758, and 759, respectively.

In certain embodiments, the anti-IGSF8 antibody or antigen binding fragment thereof comprises a VH CDR1, a VH CDR2, a VH CDR3, a VL CDR1, a VL CDR2, and a VL CDR3 that comprise, consist essentially of, or consist of the amino acid sequences of SEQ ID NOs: 760, 761, 762, 763, 764, and 765, respectively.

In certain embodiments, the anti-IGSF8 antibody or antigen binding fragment thereof comprises a VH that comprise, consist essentially of, or consist of the amino acid sequences of SEQ ID NOs: 734, 735, and 736; and a VL that comprise, consist essentially of, or consist of the amino acid sequences of SEQ ID NOs:737, 738, and 739, respectively.

In certain embodiments, the anti-IGSF8 antibody or antigen binding fragment thereof comprises a VH that comprise, consist essentially of, or consist of the amino acid sequences of SEQ ID NOs: 740, 741, and 742; and a VL that comprise, consist essentially of, or consist of the amino acid sequences of SEQ ID NOs:743, 744, and 745, respectively.

In certain embodiments, the anti-IGSF8 antibody or antigen binding fragment thereof comprises a VH that comprise, consist essentially of, or consist of the amino acid sequences of SEQ ID NOs: 774, 775, and 776; and a VL that comprise, consist essentially of, or consist of the amino acid sequences of SEQ ID NOs: 777, 778, and 779, respectively.

In certain embodiments, the anti-IGSF8 antibody or antigen binding fragment thereof comprises a VH that comprise, consist essentially of, or consist of the amino acid sequences of SEQ ID NOs: 780, 781, and 782; and a VL that comprise, consist essentially of, or consist of the amino acid sequences of SEQ ID NOs: 783, 784, and 785, respectively.

For example, in some embodiments, the anti-IGSF8 antibody or antigen-binding fragment thereof comprises:
(i) a VH CDR1 that comprises, consists essentially of, or consists of the amino acid sequence X1-X2-X3-X4-X5-X6-X7-X8 (SEQ ID NO: 714), wherein
   X1 is A, E, F, G, H, I, K, L, M, N, P, Q, R, T, V, W or Y,
   X2 is A, C, D, E, F, G, H, K, M, N, P, Q, R, T or W,
   X3 is A, C, D, E, F, G, H, K, L, M, P, Q, R, T, V, W or Y,
   X4 is A, C, D, E, F, G, H, K, M, N, P, Q, R, T or W,
   X5 is A, C, D, E, G, H, I, K, L, M, N, Q, R, S, V or W,
   X6 is C, D, E, F, G, H, I, L, N, P, Q, T, V, W or Y,
   X7 is A, D, E, F, G, I, K, L, M, P, Q, R, S, T, V, W or Y, and
   X8 is E, F, G, H, I, K, L, M, N, P, Q, R, T, W or Y;
(ii) a VH CDR2 that comprises, consists essentially of, or consists of the amino acid sequence X3-X4-X5-X6-X7-X8-X9-X10 (SEQ ID NO: 715), wherein
   X3 is A, C, D, E, G, H, I, K, L, M, P, Q, R, W or Y,
   X4 is A, D, E, F, H, I, K, M, N, P, Q, R, T, V, W or Y, e.g., R,
   X5 is C or D,
   X6 is A, D, E, F or G, e.g., G, E, or A, most preferably G,
   X7 is D, E, F, G, H, I, K, L, M, N, P, Q, T, W or Y,
   X8 is C, F, H, K, P, R, S, T, W or Y, e.g., K or R, most preferably K,
   X9 is A, D, E, F, G, I, K, L, M, P, Q, R, T, V, W or Y, and
   X10 is A, C, D, F, G, H, I, K, L, P, Q, S, V, W or Y;
(iii) a VH CDR3 that comprises, consists essentially of, or consists of the amino acid sequence X1-X2-X3-X4-X5-X6-X7-X8-X9-X10-X11-X12-X13 (SEQ ID NO: 716), wherein
   X1 is A, C, D, F, G, H, I, K, L, M, N, Q, R, W or Y,
   X2 is A, C, D, E, F, H, L, M, N, P, Q, R, V, W or Y,
   X3 is C, D, F, I or Q,
   X4 is E, F, G, H, I, K, L, M, N, P or Q,
   X5 is A, D, E, F, H, I, K, L, M, P, Q, S, T, V, W or Y,
   X6 is A, E, F, G, H, I, K, L, M, N, P, Q, R, T, W or Y,
   X7 is A, D, E, F, H, I, M, N, P, Q, S, T, V, W or Y, e.g., Y,
   X8 is A, C, D, F, G, H, I, K, L, M, N, P, Q, S, T, W or Y,
   X9 is A, E, G, I, K, L, M, P, Q, R, T, V, W or Y,
   X10 is A, C, E, F, H, I, K, L, M, N, Q or R,
   X11 is D, F, G, H, M, N, P, R, T or W,
   X12 is C, D, F, K, L, M, P, Q, R or W, e.g., R or K, and
   X13 is G, H, I, K, M, P, Q, R, W or Y;
(iv) a VL CDR1 that comprises, consists essentially of, or consists of the amino acid sequence X4-X5-X6-X7-X8-X9 (SEQ ID NO: 717), wherein
   X4 is A, C, D, E, F, G, I, K, L, M, N, Q, S, T, V, W or Y,
   X5 is A, C, D, E, F, H, I, K, L, M, N, P, Q, R, T, V or W,
   X6 is A, C, D, E, F, G, H, I, K, M, P, Q, R, V, W or Y,
   X7 is C, D, E, F, G, K, L, M, R, S, T, V, W or Y, e.g., E, G, K, M, T, V, or W,
   X8 is C, D, E, F, G, H, I, L, M, P, Q, S, T, V, W or Y, e.g., D, F, G, L, M, P, Q, S, T, V, W, or Y, and
   X9 is A, C, F, G, H, I, Q, S, T, W or Y, e.g., A, C, G, Q, S, T, or W, most preferably W;
(v) a VL CDR2 that comprises, consists essentially of, or consists of the amino acid sequence X6-X7-X8 (SEQ ID NO: 718), wherein
   X6 is A, C, D, F, G, H, N, R or S, e.g., A, G, H, N, R or S, most preferably G,
   X7 is A, C, D, I, K, S or T, e.g., D, S, or T, most preferably S, and
   X8 is A, C, D, E, F, H, I, N, P, S, T, V or W, e.g., A, D, E, F, H, N, P, T, V, or W, most preferably P
(vi) a VL CDR3 that comprises, consists essentially of, or consists of the amino acid sequence X1-X2-X3-X4-X5-X6-X7-X8-X9 (SEQ ID NO: 719), wherein
   X1 is A, C, D, E, F, G, I, M, N, P, Q, S, T, V, W or Y,
   X2 is A, C, D, E, F, G, I, M, N, P, Q, S, T, V, W or Y,
   X3 is A, C, D, E, G, I, K, L, M, N, P, Q, R, T, V, W or Y,
   X4 is D, E, F, P, Q or Y, e.g., E, Q, or Y,
   X5 is G, K, L, M, N, P, Q, R or S, e.g., G, R or K,
   X6 is C, D, E, F, H, I, L, M, N, P, Q, S, T, V or Y, e.g., D, E, L, M, N, Q, S, T, or V,
   X7 is A, C, D, E, F, G, I, K, L, M, N, P, Q, R, V, W or Y,
   X8 is A, E, F, G, I, K, M, N, P, Q, R, T, V, W or Y, and
   X9 is C, D, E, F, G, H, I, K, L, M, N, Q, R, T, V, W or Y.

As another example, in some embodiments, the anti-IGSF8 antibody or antigen-binding fragment thereof comprises:
(i) a VH CDR1 that comprises, consists essentially of, or consists of the amino acid sequence X1-X2-X3-X4-X5-X6-X7-X8 (SEQ ID NO: 720), wherein
   X1 is A, C, D, E, F, G, H, I, K, L, M, N, P, Q, R, S, T, V, W or Y, e.g., R,
   X2 is A, C, D, E, F, G, H, K, L, M, N, P, Q, R, S, T, V or W, e.g., G,
   X3 is A, C, D, E, F, G, H, I, K, L, M, N, P, Q, R, T, V, W or Y,
   X4 is A, C, D, E, F, G, H, I, K, M, N, P, Q, R, S, T, V, W or Y,
   X5 is I, K, L, M, P, Q, V, W or Y, e.g., K,
   X6 is F, G, H, I, K, L, M, P, Q, T, V, W or Y,
   X7 is A, C, D, E, F, G, H, I, K, M, N, P, Q, R, S, T, V, W or Y, e.g., F, S or N, more preferably F, and
   X8 is A, C, D, E, F, H, K, L, M, N, P, Q, R, T or V;
(ii) a VH CDR2 that comprises, consists essentially of, or consists of the amino acid sequence X2-X3-X4-X5-X6-X7-X8-X9 (SEQ ID NO: 721), wherein
   X2 is A, C, D, E, G, H, I, K, L, M, N, P, Q, R, S, T, V, W or Y,
   X3 is A, C, E, F, G, H, I, K, L, M, P, Q, R, S, V or Y,
   X4 is C, D, E, F, G, H, I, K, L, M, N, Q, R, S or V,
   X5 is A, C, F, H, K, L, M, P, Q, R, S, T, V or W, e.g., M,
   X6 is A, C, E, F, G, H, I, K, L, M, P, Q, R, V or W, e.g., F,
   X7 is A, C, D, E, F, G, H, I, K, L, M, N, P, Q, R, T, V, W or Y,
   X8 is A, C, F, G, I, K, L, M, N, P, Q, R, S, T, V, W or Y, e.g., G, N, R, S, or T, more preferably G or S, and
   X9 is C, D, E, F, G, H, K, L, M, N, P, Q, S, T, V, W or Y;
(iii) a VH CDR3 that comprises, consists essentially of, or consists of the amino acid sequence X1-X2-X3-X4-X5-X6-X7-X8-X9-X10-X11-X12-X13-X14-X15 (SEQ ID NO: 722), wherein
   X1 is A, C, D, E, F, G, H, I, K, L, M, N, P, Q, R, S, T, V, W or Y,
   X2 is F, G, H, I or T,
   X3 is A, C, D, F, G, H, I, K, L, M, N, P, Q, R, S, T, V, W or Y,
   X4 is D, E, F, H, N, Q, R, S, T, V, W or Y, e.g., D,
   X5 is A, H, I, L, M, N, Q or Y,
   X6 is A, C, D, F, G, H, K, M, N, P, Q, R, S, T, V or Y,
   X7 is A, C, E, F, H, K, M, N, P, Q, S, T, W or Y,
   X8 is A, C, D, E, F, G, H, I, K, L, M, N, P, Q, S, T, V or W,
   X9 is A, C, D, E, F, H, I, K, L, N, Q, R, S, V, W or Y,
   X10 is A, C, D, E, F, H, I, K, L, M, N, P, Q, R, S, V, W or Y,
   X11 is A, C, E, F, H, I, K, L, M, N, P, Q, S, T, V, W or Y,
   X12 is F, H, I, K, N, P, Q, R, V, W or Y, e.g., F or Y,
   X13 is A, C, D, E, F, G, H, I, K, L, N, P, Q, R, S, T, V, W or Y,
   X14 is D, F, G, H, P, Q or T, e.g., T, and
   X15 is D, E, F, G, I, K, L, N, P, Q, R, S or T;
(iv) a VL CDR1 that comprises, consists essentially of, or consists of the amino acid sequence X4-X5-X6-X7-X8-X9 (SEQ ID NO: 723), wherein
   X4 is A, C, D, E, F, G, I, K, M, N, R, S, T, V, W or Y, e.g., E,
   X5 is C, D, E, H, K, L, M, Q, T, W or Y, e.g., D,
   X6 is A, C, D, E, F, G, H, K, M, N, P, Q, R, T, V, W or Y,
   X7 is C, E, G, I, L, M, P, Q, V, W or Y,
   X8 is C, M, P, Q, T or W, e.g., P, and
   X9 is A, C, E, F, G, I, K, L, M, N, P, Q, R, T, V or Y, e.g., Y;
(v) a VL CDR2 that comprises, consists essentially of, or consists of the amino acid sequence X6-X7-X8 (SEQ ID NO: 724), wherein
   X6 is C, H, I, L, M, N, P, Q, W or Y, e.g., H or Q,
   X7 is C, D, E, F, G, H, I, K, L, M, N, P, Q, R, S, T, V, W or Y, e.g., S, T, or V, more preferably S or T, and
   X8 is C, D, E, G, H, I, K, L, M, P, Q, R, S, W or Y; and
(vi) a VL CDR3 that comprises, consists essentially of, or consists of the amino acid sequence X1-X2-X3-X4-X5-X6-X7-X8-X9 (SEQ ID NO: 725), wherein
   X1 is C, D, F, G, I, K, M, N, P, Q, S, T, V, W or Y,
   X2 is A, C, D, F, G, I, L, M, N, P, Q, R, S, T, V or W,
   X3 is C, E, G, K, M, P, S, V or W,
   X4 is C, H, L, M, P, Q, R, V or W, e.g., P,
   X5 is C, D, E, F, L, M, P, V or W, e.g., F,
   X6 is A, C, E, G, H, K, M, N, P, Q, R, V or W, e.g., A, N, P, R, or W,
   X7 is A, C, D, E, G, H, I, K, M, N, P, R, S, T, V, W or Y,
   X8 is A, C, D, E, G, K, M, N, P, Q, R, S or W, e.g., D, P, S, or W, and
   X9 is C, D, E, F, G, H, K, L, M, Q, R, T, V, W or Y.

In some embodiments, the anti-IGSF8 antibody or antigen-binding fragment thereof of the invention (e.g., those with the above-referenced consensus CDR sequences) comprises at least one, two, or three (e.g., all three) corresponding VH CDRs of any one of the antibodies listed in Tables D and G.

For example, in one embodiment, an antibody of the invention may have a VH CDR1 sequence identical to the VH CDR1 sequence of any one of the antibodies listed in Table D. In one embodiment, an antibody of the invention may have a VH CDR2 sequence identical to the VH CDR2 sequence of any one of the antibodies listed in Table D. In one embodiment, an antibody of the invention may have a VH CDR3 sequence identical to the VH CDR3 sequence of any one of the antibodies listed in Table D.

In another embodiment, an antibody of the invention may have a VH CDR1 sequence identical to the VH CDR1 sequence of any one of a first antibody listed in Table D; and a VH CDR2 sequence identical to the VH CDR2 sequence of any one of a second antibody listed in Table D, wherein the first and the second antibody are the same or different. In another embodiment, an antibody of the invention may have a VH CDR1 sequence identical to the VH CDR2 sequence of any one of a first antibody listed in Table D; and a VH CDR3 sequence identical to the VH CDR3 sequence of any one of a second antibody listed in Table D, wherein the first and the second antibody are the same or different. In another embodiment, an antibody of the invention may have a VH CDR2 sequence identical to the VH CDR2 sequence of any one of a first antibody listed in Table D; and a VH CDR3 sequence identical to the VH CDR3 sequence of any one of a second antibody listed in Table D, wherein the first and the second antibody are the same or different.

In yet another embodiment, an antibody of the invention may have a VH CDR1 sequence identical to the VH CDR1 sequence of any one of a first antibody listed in Table D; a VH CDR2 sequence identical to the VH CDR2 sequence of any one of a second antibody listed in Table D; and a VH CDR3 sequence identical to the VH CDR3 sequence of any one of a third antibody listed in Table D, wherein the first, the second, and the third antibodies are the same or different (e.g., two from the same antibody and one from another antibody, or all three from different antibodies).

In some embodiments, the anti-IGSF8 antibody or antigen-binding fragment thereof of the invention (e.g., those with the above-referenced consensus CDR sequences) comprises at least one, two, or three (e.g., all three) corresponding VH CDRs of any one of the antibodies listed in Table G.

For example, in one embodiment, an antibody of the invention may have a VH CDR1 sequence identical to the VH CDR1 sequence of any one of the antibodies listed in Table G. In one embodiment, an antibody of the invention may have a VH CDR2 sequence identical to the VH CDR2 sequence of any one of the antibodies listed in Table G. In one embodiment, an antibody of the invention may have a VH CDR3 sequence identical to the VH CDR3 sequence of any one of the antibodies listed in Table G.

In another embodiment, an antibody of the invention may have a VH CDR1 sequence identical to the VH CDR1 sequence of any one of a first antibody listed in Table G; and a VH CDR2 sequence identical to the VH CDR2 sequence of any one of a second antibody listed in Table G, wherein the first and the second antibody are the same or different. In another embodiment, an antibody of the invention may have a VH CDR1 sequence identical to the VH CDR2 sequence of any one of a first antibody listed in Table G; and a VH CDR3 sequence identical to the VH CDR3 sequence of any one of a second antibody listed in Table G, wherein the first and the second antibody are the same or different. In another embodiment, an antibody of the invention may have a VH CDR2 sequence identical to the VH CDR2 sequence of any one of a first antibody listed in Table G; and a VH CDR3 sequence identical to the VH CDR3 sequence of any one of a second antibody listed in Table G, wherein the first and the second antibody are the same or different.

In yet another embodiment, an antibody of the invention may have a VH CDR1 sequence identical to the VH CDR1 sequence of any one of a first antibody listed in Table G; a VH CDR2 sequence identical to the VH CDR2 sequence of any one of a second antibody listed in Table G; and a VH CDR3 sequence identical to the VH CDR3 sequence of any one of a third antibody listed in Table G, wherein the first, the second, and the third antibodies are the same or different (e.g., two from the same antibody and one from another antibody, or all three from different antibodies).

In some embodiments, VH CDR1, VH CDR2, and/or VH CDR3 of the anti-IGSF8 antibody or antigen-binding fragment thereof of the invention (e.g., those with the above-referenced consensus CDR sequences) each or collectively have one, two, three, four, five or more changes, e.g., amino acid substitutions, insertions, or deletions, relative to the amino acid sequences of the corresponding VH CDR1, VH CDR2, and/or VH CDR3 of any one of the antibodies listed in Table D.

In some embodiments, VH CDR1, VH CDR2, and/or VH CDR3 of the anti-IGSF8 antibody or antigen-binding fragment thereof of the invention (e.g., those with the above-referenced consensus CDR sequences) each or collectively have one, two, three, four, five or more changes, e.g., amino acid substitutions, insertions, or deletions, relative to the amino acid sequences of the corresponding VH CDR1, VH CDR2, and/or VH CDR3 of any one of the antibodies listed in Table G.

In some embodiments, the anti-IGSF8 antibody or antigen-binding fragment thereof of the invention (e.g., those with the above-referenced consensus CDR sequences) comprises at least one, two, or three (e.g., all three) corresponding VL CDRs of any one of the antibodies listed in Table D.

For example, in one embodiment, an antibody of the invention may have a VL CDR1 sequence identical to the VL CDR1 sequence of any one of the antibodies listed in Table D. In one embodiment, an antibody of the invention may have a VL CDR2 sequence identical to the VL CDR2 sequence of any one of the antibodies listed in Table D. In one embodiment, an antibody of the invention may have a VL CDR3 sequence identical to the VL CDR3 sequence of any one of the antibodies listed in Table D.

In another embodiment, an antibody of the invention may have a VL CDR1 sequence identical to the VL CDR1 sequence of any one of a first antibody listed in Table D; and a VL CDR2 sequence identical to the VL CDR2 sequence of any one of a second antibody listed in Table D, wherein the first and the second antibody are the same or different. In another embodiment, an antibody of the invention may have a VL CDR1 sequence identical to the VL CDR2 sequence of any one of a first antibody listed in Table D; and a VL CDR3 sequence identical to the VL CDR3 sequence of any one of a second antibody listed in Table D, wherein the first and the second antibody are the same or different. In another embodiment, an antibody of the invention may have a VL CDR2 sequence identical to the VL CDR2 sequence of any one of a first antibody listed in Table D; and a VL CDR3 sequence identical to the VL CDR3 sequence of any one of a second antibody listed in Table D, wherein the first and the second antibody are the same or different.

In yet another embodiment, an antibody of the invention may have a VL CDR1 sequence identical to the VL CDR1 sequence of any one of a first antibody listed in Table D; a VL CDR2 sequence identical to the VL CDR2 sequence of any one of a second antibody listed in Table D; and a VL CDR3 sequence identical to the VL CDR3 sequence of any one of a third antibody listed in Table D, wherein the first, the second, and the third antibodies are the same or different (e.g., two from the same antibody and one from another antibody, or all three from different antibodies).

In some embodiments, the anti-IGSF8 antibody or antigen-binding fragment thereof of the invention (e.g., those with the above-referenced consensus CDR sequences) comprises at least one, two, or three (e.g., all three) corresponding VL CDRs of any one of the antibodies listed in Table G.

For example, in one embodiment, an antibody of the invention may have a VL CDR1 sequence identical to the VL CDR1 sequence of any one of the antibodies listed in Table G. In one embodiment, an antibody of the invention may have a VL CDR2 sequence identical to the VL CDR2 sequence of any one of the antibodies listed in Table G. In one embodiment, an antibody of the invention may have a VL CDR3 sequence identical to the VL CDR3 sequence of any one of the antibodies listed in Table G.

In another embodiment, an antibody of the invention may have a VL CDR1 sequence identical to the VL CDR1 sequence of any one of a first antibody listed in Table G; and a VL CDR2 sequence identical to the VL CDR2 sequence of any one of a second antibody listed in Table G, wherein the first and the second antibody are the same or different. In another embodiment, an antibody of the invention may have a VL CDR1 sequence identical to the VL CDR2 sequence of any one of a first antibody listed in Table G; and a VL CDR3 sequence identical to the VL CDR3 sequence of any one of a second antibody listed in Table G, wherein the first and the second antibody are the same or different. In another embodiment, an antibody of the invention may have a VL CDR2 sequence identical to the VL CDR2 sequence of any one of a first antibody listed in Table G; and a VL CDR3 sequence identical to the VL CDR3 sequence of any one of a second antibody listed in Table G, wherein the first and the second antibody are the same or different.

In yet another embodiment, an antibody of the invention may have a VL CDR1 sequence identical to the VL CDR1 sequence of any one of a first antibody listed in Table G; a VL CDR2 sequence identical to the VL CDR2 sequence of any one of a second antibody listed in Table G; and a VL CDR3 sequence identical to the VL CDR3 sequence of any one of a third antibody listed in Table G, wherein the first, the second, and the third antibodies are the same or different (e.g., two from the same antibody and one from another antibody, or all three from different antibodies).

In some embodiments, VL CDR1, VL CDR2, and/or VL CDR3 of the anti-IGSF8 antibody or antigen-binding fragment thereof of the invention (e.g., those with the above-referenced consensus CDR sequences) each or collectively have one, two, three, four, five or more changes, e.g., amino acid substitutions, insertions, or deletions, relative to the amino acid sequences of the corresponding VL CDR1, VL CDR2, and/or VL CDR3 of any one of the antibodies listed in Table D.

In some embodiments, VL CDR1, VL CDR2, and/or VL CDR3 of the anti-IGSF8 antibody or antigen-binding fragment thereof of the invention (e.g., those with the above-referenced consensus CDR sequences) each or collectively have one, two, three, four, five or more changes, e.g., amino acid substitutions, insertions, or deletions, relative to the amino acid sequences of the corresponding VL CDR1, VL CDR2, and/or VL CDR3 of any one of the antibodies listed in Table G.

In any of the embodiments below concerning specific antibodies defined by 6 CDR region sequences, it is explicitly contemplated that the VH CDR1, VH CDR2 and VH CDR3 comprises, consists essentially of, of consists of the amino acid sequence of the respective recited SEQ ID NOs., and the VL CDR1, VL CDR2 and VL CDR3 comprises, consists essentially of, of consists of the amino acid sequence of the respective recited SEQ ID NOs. However, for simplicity, the following descriptions only use the transition phrase “comprise(s).”

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 612, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 624 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 613, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 615, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 616, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 626 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 627 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 617, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 628 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 629 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 611, 630 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 618, 623 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 629 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 619, 629 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 615, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 620, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 621, 635 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 620, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 619, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 622, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 615, 625 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 629 and 631, respectively.

In some embodiments, the anti-IGSF8 antibody comprises the VH CDR1, VH CDR2 and VH CDR3 comprising the amino acid sequence of SEQ ID NOs: 601, 602, and 605, respectively, and the VL CDR1, VL CDR2 and VL CDR3 comprising the amino acid sequence of SEQ ID NOs: 614, 628 and 631, respectively.

In some embodiments, the anti-IGSF8 antibody comprises the VH CDR1, VH CDR2 and VH CDR3 comprising the amino acid sequence of SEQ ID NOs: 601, 603 and 605, respectively, and the VL CDR1, VL CDR2 and VL CDR3 comprising the amino acid sequence of SEQ ID NOs: 614, 624 and 631, respectively.

In some embodiments, the anti-IGSF8 antibody comprises the VH CDR1, VH CDR2 and VH CDR3 comprising the amino acid sequence of SEQ ID NOs: 601, 604 and 605, respectively, and the VL CDR1, VL CDR2 and VL CDR3 comprising the amino acid sequence of SEQ ID NOs: 614, 625 and 631, respectively.

In some embodiments, the anti-IGSF8 antibody comprises the VH CDR1, VH CDR2 and VH CDR3 comprising the amino acid sequence of SEQ ID NOs: 601, 603 and 605, respectively, and the VL CDR1, VL CDR2 and VL CDR3 comprising the amino acid sequence of SEQ ID NOs: 614, 625 and 631, respectively.

In some embodiments, the anti-IGSF8 antibody comprises the VH CDR1, VH CDR2 and VH CDR3 comprising the amino acid sequence of SEQ ID NOs: 643, 644 and 646, respectively, and the VL CDR1, VL CDR2 and VL CDR3 comprising the amino acid sequence of SEQ ID NOs: 652, 653 and 655, respectively.

In some embodiments, the anti-IGSF8 antibody comprises the VH CDR1, VH CDR2 and VH CDR3 comprising the amino acid sequence of SEQ ID NOs: 643, 644 and 646, respectively, and the VL CDR1, VL CDR2 and VL CDR3 comprising the amino acid sequence of SEQ ID NOs: 652, 654, and 655, respectively.

In some embodiments, the anti-IGSF8 antibody comprises the VH CDR1, VH CDR2 and VH CDR3 comprising the amino acid sequence of SEQ ID NOs: 643, 645 and 646, respectively, and the VL CDR1, VL CDR2 and VL CDR3 comprising the amino acid sequence of SEQ ID NOs: 652, 653 and 655 respectively.

In some embodiments, the anti-IGSF8 antibody comprises the VH CDR1, VH CDR2 and VH CDR3 comprising the amino acid sequence of SEQ ID NOs: 643, 645 and 646, respectively, and the VL CDR1, VL CDR2 and VL CDR3 comprising the amino acid sequence of SEQ ID NOs: 652, 654 and 655 respectively.

### Framework Regions (FRs)

Anti-IGSF antibodies or antigen-binding fragments thereof according to the present disclosure may be prepared using any of the framework region (FR) of amino acid sequences as described in Table D and/or Table G, or sequences substantially identical (e.g., having at least about 80%, 85%, 90%, 92%, 95%, 97%, 98%, or 99% sequence identity) to the FR amino acid sequences as described in Table D and/or Table G.

In some embodiments, the anti-IGSF8 antibody or antigen-binding fragment thereof has a heavy chain variable region (VH) comprising one, two, three, or all (i.e., four) of a heavy chain framework region 1 (VH FR1), a heavy chain framework region 2 (VH FR2), a heavy chain framework region 3 (VH FR3), and/or a heavy chain framework region 4 (VH FR4) of the corresponding heavy chain framework regions of any one of the antibodies listed in Table D or G, or a VH FR1, VH FR2, VH FR3 and/or VH FR4 comprising sequences substantially identical (e.g., having at least about 80%, 85%, 90%, 92%, 95%, 97%, 98%, or 99% sequence identity) to the corresponding VH FR amino acid sequences of any one of the antibodies as described in Table D or Table G.

In some embodiments, anti-IGSF8 antibody comprises a VH FR1 of SEQ ID NO: 606, 647 or 648, or an amino acid sequence substantially identical (e.g., having at least about 80%, 85%, 90%, 92%, 95%, 97%, 98%, or 99% sequence identity) to SEQ ID NO: 606, 647 or 648.

In some embodiments, anti-IGSF8 antibody comprises a VH FR2 of SEQ ID NO: 607, 649 or 650, or an amino acid sequence substantially identical (e.g., having at least about 80%, 85%, 90%, 92%, 95%, 97%, 98%, or 99% sequence identity) to SEQ ID NO: 607, 649 or 650.

In some embodiments, anti-IGSF8 antibody comprises a VH FR3 of SEQ ID NO: 608 or 651, or an amino acid sequence substantially identical (e.g., having at least about 80%, 85%, 90%, 92%, 95%, 97%, 98%, or 99% sequence identity) to SEQ ID NO: 608 or 651.

In some embodiments, anti-IGSF8 antibody comprises a VH FR4 of SEQ ID NO: 609 or 610, or an amino acid sequence substantially identical (e.g., having at least about 80%, 85%, 90%, 92%, 95%, 97%, 98%, or 99% sequence identity) to SEQ ID NO:60 9 or 610.

In some embodiments, the anti-IGSF8 antibody has a VH comprising one, two, three, or all of an VH FR1, VH FR2, VH FR3 and/or VH FR4 comprising the amino acid sequence of SEQ ID NOs: 606, 607, 608 and/or 609, respectively, or an amino acid sequence substantially identical (e.g., having at least about 80%, 85%, 90%, 92%, 95%, 97%, 98%, or 99% sequence identity) to SEQ ID NOs:60 6, 607, 608, and/or 609.

In some embodiments, the anti-IGSF8 antibody has a VH comprising one, two, three, or all of an VH FR1, VH FR2, VH FR3 and/or VH FR4 comprising the amino acid sequence of SEQ ID NOs: 606, 607, 608 and/or 609, respectively, or an amino acid sequence substantially identical (e.g., having at least about 80%, 85%, 90%, 92%, 95%, 97%, 98%, or 99% sequence identity) to SEQ ID NOs: 606, 607, 608, and/or 610.

In some embodiments, the anti-IGSF8 antibody has a VH comprising one, two, three, or all of an VH FR1, VH FR2, VH FR3 and/or VH FR4 comprising the amino acid sequence of SEQ ID NOs: 647, 649, 651 and/or 610, respectively, or an amino acid sequence substantially identical (e.g., having at least about 80%, 85%, 90%, 92%, 95%, 97%, 98%, or 99% sequence identity) to SEQ ID NOs: 647, 649, 651 and/or 610.

In some embodiments, the anti-IGSF8 antibody has a VH comprising one, two, three, or all of an VH FR1, VH FR2, VH FR3 and/or VH FR4 comprising the amino acid sequence of SEQ ID NOs: 648, 649, 651 and/or 610, respectively, or an amino acid sequence substantially identical (e.g., having at least about 80%, 85%, 90%, 92%, 95%, 97%, 98%, or 99% sequence identity) to SEQ ID NOs: 648, 649, 651 and/or 610.

In some embodiments, the anti-IGSF8 antibody has a VH comprising one, two, three, or all of an VH FR1, VH FR2, VH FR3 and/or VH FR4 comprising the amino acid sequence of SEQ ID NOs: 648, 650, 651 and/or 610, respectively, or an amino acid sequence substantially identical (e.g., having at least about 80%, 85%, 90%, 92%, 95%, 97%, 98%, or 99% sequence identity) to SEQ ID NOs: 648, 650, 651 and/or 610.

In some embodiments, the anti-IGSF8 antibody or antigen-binding fragment thereof has a light chain variable region (VL) comprising one, two, three, or all (i.e., four) of a light chain framework region 1 (VL FR1), a light chain framework region 2 (VL FR2), a light chain framework region 3 (VL FR3), and/or a light chain framework region 4 (VL FR4) of the corresponding light chain framework regions of any one of the antibodies listed in Table D or G, or a VL FR1, VL FR2, VL FR3 and/or VL FR4 comprising sequences substantially identical (e.g., having at least about 80%, 85%, 90%, 92%, 95%, 97%, 98%, or 99% sequence identity) to the corresponding VL FR amino acid sequences of any one of the antibodies as described in Table D or Table G.

In some embodiments, anti-IGSF8 antibody comprises a VL FR1 of SEQ ID NO: 632, 633, 656 or 657, or an amino acid sequence substantially identical (e.g., having at least about 80%, 85%, 90%, 92%, 95%, 97%, 98%, or 99% sequence identity) to SEQ ID NO: 632, 633, 656 or 657.

In some embodiments, anti-IGSF8 antibody comprises a VL FR2 of SEQ ID NO: 634, 635, 636, 637, 658 or 659, or an amino acid sequence substantially identical (e.g., having at least about 80%, 85%, 90%, 92%, 95%, 97%, 98%, or 99% sequence identity) to SEQ ID NO: 634, 635, 636, 637, 658 or 659.

In some embodiments, anti-IGSF8 antibody comprises a VL FR3 of SEQ ID NO: 638, 639, 640, 660, 661, 662 or 663, or an amino acid sequence substantially identical (e.g., having at least about 80%, 85%, 90%, 92%, 95%, 97%, 98%, or 99% sequence identity) to SEQ ID NO: 638, 639, 640, 660, 661, 662 or 663.

In some embodiments, anti-IGSF8 antibody comprises a VL FR4 of SEQ ID NO: 641, 642, 664 or 665, or an amino acid sequence substantially identical (e.g., having at least about 80%, 85%, 90%, 92%, 95%, 97%, 98%, or 99% sequence identity) to SEQ ID NO: 641, 642, 664 or 665.

In some embodiments, the anti-IGSF8 antibody has a VL comprising one, two, three, or all of an VL FR1, VL FR2, VL FR3 and/or VL FR4 comprising the amino acid sequence of SEQ ID NOs: 632, 634, 638 and/or 641, respectively, or an amino acid sequence substantially identical (e.g., having at least about 80%, 85%, 90%, 92%, 95%, 97%, 98%, or 99% sequence identity) to SEQ ID NOs: 632, 634, 638 and/or 641.

In some embodiments, the anti-IGSF8 antibody has a VL comprising one, two, three, or all of an VL FR1, VL FR2, VL FR3 and/or VL FR4 comprising the amino acid sequence of SEQ ID NOs: 633, 635, 639 and/or 642, respectively, or an amino acid sequence substantially identical (e.g., having at least about 80%, 85%, 90%, 92%, 95%, 97%, 98%, or 99% sequence identity) to SEQ ID NOs: 633, 635, 639 and/or 642.

In some embodiments, the anti-IGSF8 antibody has a VL comprising one, two, three, or all of an VL FR1, VL FR2, VL FR3 and/or VL FR4 comprising the amino acid sequence of SEQ ID NOs: 632, 635, 639 and/or 64247, respectively, or an amino acid sequence substantially identical (e.g., having at least about 80%, 85%, 90%, 92%, 95%, 97%, 98%, or 99% sequence identity) to SEQ ID NOs: 632, 635, 639 and/or 642.

In some embodiments, the anti-IGSF8 antibody has a VL comprising one, two, three, or all of an VL FR1, VL FR2, VL FR3 and/or VL FR4 comprising the amino acid sequence of SEQ ID NOs: 632, 636, 639 and/or 642, respectively, or an amino acid sequence substantially identical (e.g., having at least about 80%, 85%, 90%, 92%, 95%, 97%, 98%, or 99% sequence identity) to SEQ ID NOs: 632, 636, 639 and/or 642.

In some embodiments, the anti-IGSF8 antibody has a VL comprising one, two, three, or all of an VL FR1, VL FR2, VL FR3 and/or VL FR4 comprising the amino acid sequence of SEQ ID NOs: 632, 637, 640 and/or 642, respectively, or an amino acid sequence substantially identical (e.g., having at least about 80%, 85%, 90%, 92%, 95%, 97%, 98%, or 99% sequence identity) to SEQ ID NOs: 632, 637, 639 and/or 642.

In some embodiments, the anti-IGSF8 antibody has a VL comprising one, two, three, or all of an VL FR1, VL FR2, VL FR3 and/or VL FR4 comprising the amino acid sequence of SEQ ID NOs: 656, 658, 660 and/or 664, respectively, or an amino acid sequence substantially identical (e.g., having at least about 80%, 85%, 90%, 92%, 95%, 97%, 98%, or 99% sequence identity) to SEQ ID NOs: 656, 658, 660 and/or 664.

In some embodiments, the anti-IGSF8 antibody has a VL comprising one, two, three, or all of an VL FR1, VL FR2, VL FR3 and/or VL FR4 comprising the amino acid sequence of SEQ ID NOs: 657, 659, 661 and/or 665, respectively, or an amino acid sequence substantially identical (e.g., having at least about 80%, 85%, 90%, 92%, 95%, 97%, 98%, or 99% sequence identity) to SEQ ID NOs: 657, 659, 661 and/or 665.

In some embodiments, the anti-IGSF8 antibody has a VL comprising one, two, three, or all of an VL FR1, VL FR2, VL FR3 and/or VL FR4 comprising the amino acid sequence of SEQ ID NOs: 657, 659, 662 and/or 665, respectively, or an amino acid sequence substantially identical (e.g., having at least about 80%, 85%, 90%, 92%, 95%, 97%, 98%, or 99% sequence identity) to SEQ ID NOs: 657, 659, 662 and/or 665.

In some embodiments, the anti-IGSF8 antibody has a VL comprising one, two, three, or all of an VL FR1, VL FR2, VL FR3 and/or VL FR4 comprising the amino acid sequence of SEQ ID NOs: 657, 659, 663 and/or 665, respectively, or an amino acid sequence substantially identical (e.g., having at least about 80%, 85%, 90%, 92%, 95%, 97%, 98%, or 99% sequence identity) to SEQ ID NOs: 657, 659, 663 and/or 665.

In some embodiments, the monoclonal antibody or antigen-binding fragment thereof is a human-mouse chimeric antibody, a humanized antibody, a human antibody, a CDR-grafted antibody, or a resurfaced antibody.

In some embodiments, the antigen-binding fragment thereof is an Fab, Fab′, F(ab′)₂, F_{d}, single chain Fv or scFv, disulfide linked F, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In some embodiments, the monoclonal antibody or antigen-binding fragment thereof binds IGSF8 with a K_{d} of less than about 25 nM, 20 nM, 15 nM, 10 nM, 5 nM, 2 nM, or 1 nM.

In some embodiments, an antibody binds to IGSF8 from multiple species. For example, in some embodiments, an antibody binds to human IGSF8, and also binds to IGSF8 from at least one non-human mammal selected from mouse, rat, dog, guinea pig, and cynomolgus monkey.

In some embodiments, multispecific antibodies are provided. In some embodiments, bispecific antibodies are provided. Non-limiting exemplary bispecific antibodies include antibodies comprising a first arm comprising a heavy chain/light chain combination that binds a first antigen and a second arm comprising a heavy chain/light chain combination that binds a second antigen. A further non-limiting exemplary multispecific antibody is a dual variable domain antibody. In some embodiments, a bispecific antibody comprises a first arm that inhibits binding of IGSF8 and a second arm that stimulates T cells, e.g., by binding CD3. In some embodiments, the first arm binds IGSF8.

Another aspect of the invention provides a monoclonal antibody or an antigen-binding fragment thereof, which competes with the monoclonal antibody or antigen-binding fragment thereof of the invention described herein above.

In certain embodiments, the antibody or antigen-binding portion/fragment thereof specifically binds the D1 ECD (or Ig-V set domain) of IGSF8, preferably with a K_{D} of no more than 5 nM, 2 nM, or 1 nM.

In certain embodiments, the antibody or antigen-binding portion/fragment thereof inhibits IGSF8 binding to KIR3DL1/2.

In certain embodiments, the antibody or antigen-binding portion/fragment thereof inhibits IGSF8 binding to the D2 domain of KIR3DL1/2, such as an epitope comprising 5165, I171, and/or M186 of KIR3DL1/2.

Another aspect of the invention provides a monoclonal antibody or an antigen-binding portion/fragment thereof, which specifically binds the D1 ECD (or Ig-V set domain) of IGSF8, and inhibits binding to KIR3DL1/2, such as binding to the D2 domain of KIR3DL1/2 (e.g., an epitope comprising 5165, I171, and/or M186 of KIR3DL1/2).

In some embodiments, the monoclonal antibody or antigen-binding portion/fragment thereof has a K_{D} of no more than 5 nM, 2 nM, or 1 nM.

In a related aspect, the invention also provides a polynucleotide encoding a monoclonal antibody of the invention, a heavy chain or a light chain thereof, or an antigen-binding portion/fragment thereof. See separate section below.

In a related aspect, the invention also provides a polynucleotide that hybridizes under stringent conditions with the polynucleotide of the invention, or with a complement thereof.

In a related aspect, the invention also provides a vector comprising the polynucleotide of the invention. See separate section below.

In a related aspect, the invention also provides a host cell comprising the polynucleotide of the invention, or the vector of the invention, for expressing the encoded monoclonal antibody, heavy or light chain thereof, or antigen-binding portion/fragment thereof. See separate section below.

In a related aspect, the invention also provides a method of producing the monoclonal antibody, heavy or light chain thereof, or antigen-binding portion/fragment thereof of the invention, the method comprising: (i) culturing the host cell of the invention capable of expressing said monoclonal antibody, heavy or light chain thereof, or antigen-binding portion/fragment thereof under a condition suitable to express said monoclonal antibody, heavy or light chain thereof, or antigen-binding portion/fragment thereof, and (ii) recovering/isolating/purifying the expressed monoclonal antibody, heavy or light chain thereof, or antigen-binding portion/fragment thereof.

In a related aspect, the invention also provides a device or kit comprising at least one antibody, monoclonal antibody, heavy or light chain thereof, or antigen-binding portion/fragment thereof, of the invention, said device or kit optionally comprising a label to detect said at least one antibody, monoclonal antibody, heavy or light chain thereof, or antigen-binding portion/fragment thereof, or a complex comprising said at least one antibody, monoclonal antibody, heavy or light chain thereof, or antigen-binding portion/fragment thereof.

Anti-IGSF8 antibodies according to the present disclosure may be prepared using any of the antibody sequences (e.g., variable domain amino acid sequences, variable domain amino acid sequence pairs, CDR amino acid sequences, variable domain CDR amino acid sequence sets, variable domain CDR amino acid sequence set pairs, and/or framework region amino acid sequences) presented herein, any may be prepared, for example, as monoclonal antibodies, multispecific antibodies, chimeric antibodies, antibody mimetics, scFvs, or antibody fragments.

### KIR3DL1/2 Antibodies

One aspect of the invention provides a monoclonal antibody specific for KIR3DL1/2. In certain embodiments, the monoclonal antibody is specific for the extracellular domain (ECD) of KIR3DL1/2. In certain embodiments, the monoclonal antibody is specific for the second Ig-like extracellular domain (D2 domain) of KIR3DL1/2 responsible for IGSF8 binding. In some embodiments, antibodies that block binding to IGSF8 are provided. In certain embodiments, the anti-KIR3DL1/2 monoclonal antibody inhibits IGSF8 binding to KIR3DL2 and/or KIR3DL1, such as inhibiting IGSF8 binding to residues S165, I171, and/or M186 of KIR3DL1/2.

In certain embodiments, the monoclonal antibody is specific for human KIR3DL1/2. In some embodiments, the anti-KIR3DL1/2 antibody inhibits IGSF8-mediated signaling through KIR3DL1/2. In certain embodiments, the monoclonal antibody competes with any one of the anti-KIR3DL1/2 antibodies for binding to IGSF8.

In certain embodiments, the anti-KIR3DL1/2 antibody is a human-mouse chimeric antibody, a humanized antibody, a human antibody, a CDR-grafted antibody, or a resurfaced antibody.

In certain embodiments, the antigen-binding fragment thereof is an Fab, Fab′, F(ab′)₂, F_{d}, single chain Fv or scFv, disulfide linked F, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the monoclonal antibody or antigen-binding fragment thereof binds KIR3DL1/2 with a K_{d} of less than about 25 nM, 20 nM, 15 nM, 10 nM, 5 nM, 2 nM, or 1 nM.

A related aspect provides a monoclonal antibody or an antigen-binding fragment thereof, which competes with the monoclonal antibody or antigen-binding fragment thereof of the invention for binding to KIR3DL1/2.

In certain embodiments, the antibody or antigen-binding portion/fragment thereof specifically binds the second/middle/D2 ECD of KIR3DL1/2, preferably with a K_{D} of no more than 5 nM, 2 nM, or 1 nM.

In certain embodiments, the antibody or antigen-binding portion/fragment thereof inhibits IGSF8 binding to KIR3DL1/2.

Another aspect of the invention provides a monoclonal antibody or an antigen-binding portion/fragment thereof, which specifically binds the middle/D2 ECD of KIR3DL1/2 (e.g., specifically binds an epitope comprising residues S165, I171, and/or M186), which inhibits IGSF8 binding to KIR3DL1/2.

In certain embodiments, the monoclonal antibody or antigen-binding portion/fragment thereof has a K_{D} of no more than 5 nM, 2 nM, or 1 nM.

### 7. Humanized Antibodies

In some embodiments, the IGSF8 antibody is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

An antibody may be humanized by any standard method. Non-limiting exemplary methods of humanization include methods described, e.g., in U.S. Pat. Nos. 5,530,101; 5,585,089; 5,693,761; 5,693,762; 6,180,370; Jones et al., Nature 321:522-525 (1986); Riechmann et al, Nature 332: 323-27 (1988); Verhoeyen et al, Science 239: 1534-36 (1988); and U.S. Publication No. US 2009/0136500. All incorporated by reference.

A humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody also comprises a human heavy chain constant region and/or a human light chain constant region.

### 8. Chimeric Antibodies

In some embodiments, the IGSF8 antibody is a chimeric antibody. In some embodiments, the IGSF8 antibody comprises at least one non-human variable region and at least one human constant region. In some such embodiments, all of the variable regions of the IGSF8 antibody are non-human variable regions, and all of the constant regions of the IGSF8 antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al., Proc. Natl. Acad. Sci. USA 81: 6851-55 (1984).

### 9. Human Antibodies

In some embodiments, the IGSF8 antibody is a human antibody. Human antibodies can be made by any suitable method. Non-limiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al, Nature 362: 255-8 (1993); onberg et al, Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Non-limiting exemplary methods also include making human antibodies using phage display libraries. See, e.g., Hoogenboom et al., J. Mol. Biol. 227: 381-8 (1992); Marks et al, J. Mol. Biol. 222: 581-97 (1991); and PCT Publication No. WO 99/10494.

### Human Antibody Constant Regions

In some embodiments, a humanized, chimeric, or human antibody described herein comprises one or more human constant regions. In some embodiments, the human heavy chain constant region is of an isotype selected from IgA, IgG, and IgD. In some embodiments, the human light chain constant region is of an isotype selected from K and X. In some embodiments, an antibody described herein comprises a human IgG constant region, for example, human IgG1, IgG2, IgG3, or IgG4. In some embodiments, an antibody or Fc fusion partner comprises a C237S mutation, for example, in an IgG1 constant region. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1):105-108 (1993). In some embodiments, an antibody described herein comprises a human IgG4 constant region and a human κlight chain.

The choice of heavy chain constant region can determine whether or not an antibody will have effector function in vivo. Such effector function, in some embodiments, includes antibody-dependent cell-mediated cytotoxicity (ADCC), complement-dependent cytotoxicity (CDC) and/or antibody-dependent cellular phagocytosis (ADCP), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of inflammatory conditions and/or autoimmune disorders. In some such embodiments, a human IgG4 or IgG2 heavy chain constant region is selected or engineered. In some embodiments, an IgG4 constant region comprises an S241P mutation.

In some other embodiments, effector function may not be desirable when the purpose of the antibody is to block interaction between receptor and ligand but the depletion of the target cell is not desired. In some such embodiments, heavy chain constant region with Fc deficient in effector function is selected or engineered. Non-limiting examples of Fc with reduced effector function and mutations conferring reduced effector function to Fc are described in, e.g. Liu et al. Antibodies 9:64 (2020), the entire content of which is incorporated herein by reference.

In some embodiments, the mutations conferring a reduced effector function are L234A/L235A mutations in the C1q binding site. In some embodiments, the heavy chain constant region with reduced effector function is a human IgG1 or IgG4 comprising the L234A/L235A mutations, also known as IgG1-L234A/L235A (IgG1-LALA) or IgG4-L234A/L235A (IgG4-LALA), respectively.

In some embodiments, the mutation conferring a reduced effector function is a P329G mutation that is able to descript interaction between a human IgG and a human FcTR. In some embodiments, the mutations conferring a reduced effector function are L234A/L235A/P329G. In some embodiments, the heavy chain constant region with reduced effector function is a human IgG1 comprising the L234A/L235A/P329G mutations, also known as IgG1-L234A/L235A/P329G (IgG1-LALA-PG).

In some embodiments, the mutation conferring a reduced effector function is a N297A, N297Q or N297G mutation, which removes a glycan central to the binding between human IgG and C1q and FcTRs. In some embodiments, the heavy chain constant region with reduced effector function is a human IgG1 comprising the N297A, N297Q or N297G mutation, also known as IgG1-N297A/Q/G (IgG1-NA).

In some embodiments, the mutations conferring a reduced effector function are L235A/G237A/E318A mutations. In some embodiments, the heavy chain constant region with reduced effector function is a human IgG1 comprising the L235A/G237A/E318A mutations, also known as IgG1-L235A/G237A/E318A (IgG1-AAA).

In some embodiments, the mutations conferring a reduced effector function are G236R/L328R that may lead to a reduction or complete abrogation of binding to multiple FcTRs. In some embodiments, the heavy chain constant region with reduced effector function is a human IgG1 comprising the G236R/L328R mutations, also known as IgG1-G236R/L328R (IgG1-RR).

In some embodiments, the mutations conferring a reduced effector function are S298G/T299A mutations that may abolish or significantly reduced binding to C1q and most FcTRs. In some embodiments, the heavy chain constant region with reduced effector function is a IgG1 comprising the S298G/T299A mutations, also known as IgG1-S298G/T299A (IgG1-GA), In some embodiments, the mutations conferring a reduced effector function are L234F/L235E/P331S mutations that may lead to reduced binding to low affinity FcTRs and no detectable binding to FcTRI. In some embodiments, the heavy chain constant region with reduced effector function is a human IgG1 comprising the L234F/L235E/P331S mutations, also known as IgG1-L234F/L235E/P331S (IgG1-FES).

In some embodiments, the mutations conferring a reduced effector function are L234F/L235E/D265A mutations that may lead to potent silencing of Fc region. In some embodiments, the heavy chain constant region with reduced effector function is a human IgG1 comprising the L234F/L235E/D265A mutations, also known as IgG1-L234F/L235E/D265A (IgG1-FEA).

In some embodiments, the mutations conferring a reduced effector function are E233P/L234V/L235A/G236del/S267K mutations that may lead to no binding to multiple FcTRs. In some embodiments, the heavy chain constant region with reduced effector function is a human IgG1 comprising the E233P/L234V/L235A/G236del/S267K mutations, also known as IgG1-E233P/L234V/L235A/G236del/S267K.

In some embodiments, the mutations conferring a reduced effector function are 228P/L235E mutations that prevent F9ab) arm exchange in human IgG4. In some embodiments, the heavy chain constant region with reduced effector function is a human IgG4 comprising the 228P/L235E mutations, also known as IgG4-S228P/L235E (IgG4-PE), In some embodiments, the mutations conferring a reduced effector function are H268Q/V309L/A30S/P331S mutations. In some embodiments, the heavy chain constant region with reduced effector function is a human IgG2 comprising the H268Q/V309L/A30S/P331S mutations, also known as IgG2-H268Q/V309L/A30S/P331S (IgG2m4) In some embodiments, the mutations conferring a reduced effector function are V234A/G237A/P238S/H268A/V309L/A330S/P331S mutations. In some embodiments, the heavy chain constant region with reduced effector function is a human IgG2 comprising the V234A/G237A/P238S/H268A/V309L/A330S/P331S mutations, also known as IgG2-V234A/G237A/P238S/H268A/V309L/A330S/P331S (IgG2c4d).

Any of the antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

In some embodiments, hydrophobic interactive chromatography (HIC), for example, a butyl or phenyl column, is also used for purifying some polypeptides. Many methods of purifying polypeptides are known in the art.

Alternatively, in some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al, Biotechnol. Adv. 21: 695-713 (2003).

### 10. Antibody Properties

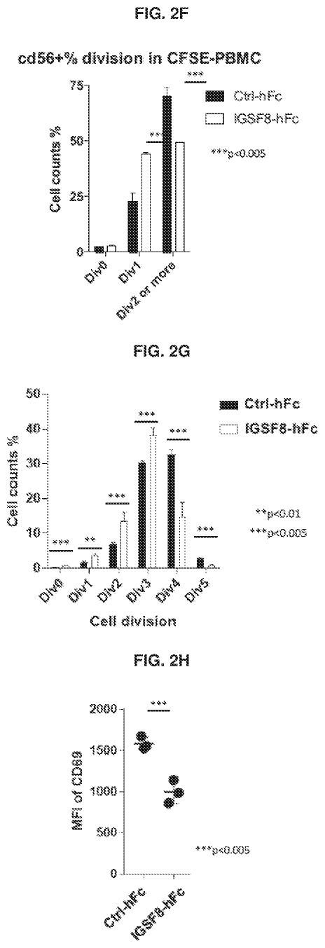

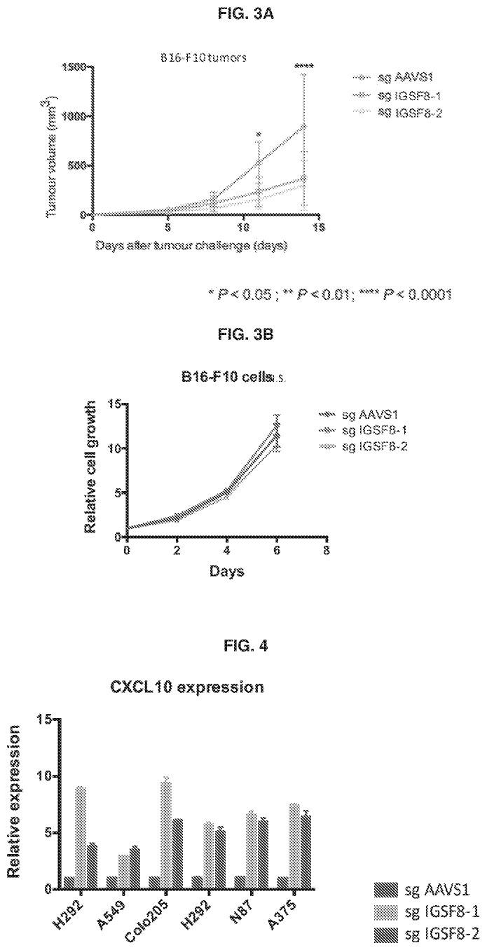

In some embodiments, the subject IGSF8 antibody binds to IGSF8 and inhibits IGSF8-mediated signaling, such as up- or down-regulation of the downstream genes as indicated in FIGS. 4, and 5A-5D. In some embodiments, IGSF8 antibody binds to IGSF8 with a binding affinity (K_{D}) or EC50 value of less than 50 nM, less than 20 nM, less than 10 nM, or less than 1 nM. In some embodiments, the extent of binding of IGSF8 antibody to an unrelated, non-IGSF8 protein is less than about 10% of the binding of the antibody to IGSF8 as measured, e.g., by a radioimmunoassay (RIA). In some embodiments, IGSF8 antibody binds to an epitope of IGSF8 that is conserved among IGSF8 from different species. In some embodiments, IGSF8 antibody binds to the same epitope as a human or humanized IGSF8 antibody that binds humIGSF8.

In some embodiments, the IGSF8 antibody is conjugated to a label, which is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

In some embodiments, a label is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, IL), Prozyme (Hayward, CA), SACRI Antibody Services (Calgary, Canada), AbD Serotec (Raleigh, NC), etc. In some embodiments, when a label is a polypeptide, the label can be expressed from the same expression vector with at least one antibody chain to produce a polypeptide comprising the label fused to an antibody chain.

### 11. IGSF8 ECDs, Fusions, and Small Peptides

In some embodiments, the IGSF8 antagonist is an IGSF8 polypeptide, such as a full-length IGSF8, or a fragment thereof that inhibits binding of IGSF8 to its ligand.

In some embodiments, the IGSF8 fragment is an IGSF8 extracellular domain (ECD). In some embodiments, the IGSF8 fragment is a full-length IGSF8 ECD. In certain embodiments, the ECD functions as a antagonistic polypeptide that inhibits the function of an IGSF8 receptor, such as KIR3dL1/2, that results from wild-type IGSF8 binding. In other embodiments, however, the ECD functions as an agonist polypeptide that functions similarly as the wild-type full-length IGSF8 on its receptor, such as KIR3DL1/2.

In some embodiments, the invention provides an IGSF8 ECD fragment, for example, comprising at least 80%, at least 85%, at least 90%, or at least 95% of the full length IGSF8 ECD amino acid sequence from which it is derived. In some embodiments, the IGSF8 ECD fragment comprises, consists essentially of, or consists of the D1 (or the most N-terminal Ig-V set) domain of IGSF8.

In some embodiments, the invention provides an IGSF8 ECD variant, for example, comprising at least 80%, at least 85%, at least 90%, at least 92%, at least 95%, at least 97%, at least 98%, or at least 99% sequence identity with the full length IGSF8 ECD or fragment (e.g., the Ig-V set D1 domain) from which it is derived. In some embodiments, the variant retains the ability to bind KIR3DL1/2.

In other embodiments, the IGSF8 ECD is from a non-human IGSF8 ECD and may be either full length, a fragment (e.g., the D1 or Ig-V set domain), or a variant (e.g., one with at least 80%, at least 85%, at least 90%, at least 92%, at least 95%, at least 97%, at least 98%, or at least 99% sequence identity and retains the ability to bind KIR3DL1/2).

In a related embodiment, the invention provides an IGSF8 variant that lacks the D2-D4 Ig-like C2 domains of the ECD, but retains the D1 Ig-V set domain of the ECD. Such variant may substantially maintain the functions of wt IGSF8, such as the ability to bind KIR3DL1/2.

In some embodiments, the IGSF8 or IGSF8 fragment or IGSF8 variant is combined with at least one fusion partner.

Thus, in some such embodiments, the invention provides a fusion of full-length IGSF8, such as a C-terminal fusion with an Ig Fc region. In one embodiment, the Ig Fc fusion is a human IgG1 Fc fusion.

The invention further provides a full length IGSF8 ECD and at least one fusion partner to form a IGSF8 ECD fusion molecule. In some embodiments, the IGSF8 ECD portion of the fusion molecule comprises a IGSF8 ECD fragment, for example, comprising at least 80%, at least 85%, at least 90%, or at least 95% of the full length IGSF8 ECD amino acid sequence from which it is derived (e.g., the D1 or Ig-V set domain). In some embodiments, the IGSF8 ECD portion of the fusion molecule is a IGSF8 ECD variant, for example, comprising at least 80%, at least 85%, at least 90%, at least 92%, at least 95%, at least 97%, at least 98%, or at least 99% sequence identity with the full length IGSF8 ECD (or the D1 or Ig-V set domain) from which it is derived, which maintains binding to KIR3DL1/2.

In other embodiments, the IGSF8 component is from a non-human IGSF8 and may be full length, a fragment (e.g., ECD), or a variant.

In any of the fusion molecule embodiments above, the fusion partner may comprise an immunoglobulin Fc molecule, for example, a human Fc molecule (e.g., human IgG1 Fc). In other embodiments, the fusion partner may be a different molecule such as albumin or polyethylene glycol (PEG). In some embodiments, more than one fusion partner may be attached to the IGSF8 or ECD thereof. In some embodiments, the fusion partner (or partners) is attached at the C-terminal, while other attachments are also possible such as on an amino acid side-chain or at the N-terminus. The attachment of a fusion partner to IGSF8 or fragments (e.g., ECD) or variants may be direct (i.e. by a covalent bond) or indirect through a linker. A linker may comprise, for example, at least one intervening amino acid or some other chemical moiety serving to link the fusion partner to the ECD either covalently or noncovalently.

In any of the above embodiments, the IGSF8 polypeptide may either include a signal sequence or be in a mature form, i.e., not including a signal sequence. The signal sequence may be from a native IGSF8 molecule or it may be a signal sequence from a different protein, for example one chosen to enhance expression of the IGSF8 polypeptide in cell culture.

In some embodiments a IGSF8 ECD may comprise the following sequence:

(SEQ ID NO: 468)
REVLVPEGPLYRVAGTAVSISCNVTGYEGPAQQNFEWFLYRPEAPDTALG
IVSTKDTQFSYAVFKSRVVAGEVQVQRLQGDAVVLKIARLQAQDAGIYEC
HTPSTDTRYLGSYSGKVELRVLPDVLQVSAAPPGPRGRQAPTSPPRMTVH
EGQELALGCLARTSTQKHTHLAVSFGRSVPEAPVGRSTLQEVVGIRSDLA
VEAGAPYAERLAAGELRLGKEGTDRYRMVVGGAQAGDAGTYHCTAAEWIQ
DPDGSWAQIAEKRAVLAHVDVQTLSSQLAVTVGPGERRIGPGEPLELLCN
VSGALPPAGRHAAYSVGWEMAPAGAPGPGRLVAQLDTEGVGSLGPGYEGR
HIAMEKVASRTYRLRLEAARPGDAGTYRCLAKAYVRGSGTRLREAASARS
RPLPVHVREEGVVLEAVAWLAGGTVYRGETASLLCNISVRGGPPGLRLAA
SWWVERPEDGELSSVPAQLVGGVGQDGVAELGVRPGGGPVSVELVGPRSH
RLRLHSLGPEDEGVYHCAPSAWVQHADYSWYQAGSARSGPVTVYPYMHAL
DT

In any of the above cases, an IGSF8 ECD may be part of a fusion molecule such that the above amino acid sequence may be joined to a fusion partner either directly or via a linker, such as an Fc, albumin, or PEG. For example, in some embodiments the IGSF8 ECD fusion molecule may comprise one of the above sequences plus an immunoglobulin Fc sequences, or an Fc from human IgG1. An IGSF8 ECD Fc fusion molecule may be formed by a direct attachment of the IGSF8 ECD amino acid sequence to the Fc amino acid sequence or via a linker (either an intervening amino acid or amino acid sequence or another chemical moiety).

In a related aspect, the invention provides a method of down-regulating NK and/or T-cell function, viability, and/or activation, comprising contacting the NK and/or T cell with an IGSF8 polypeptide of the invention, or a fusion thereof.

In a related aspect, the invention provides a method of treating a disease or condition, such as autoimmune disease or excessive inflammatory response (e.g., as in chronic inflammatory diseases) mediated by NK cell and/or T-cell activation, comprising contacting the NK cell and/or T cell with an IGSF8 polypeptide of the invention, or a fusion thereof.

In certain embodiments, the autoimmune disease is associated with excessive NK cell and/or T cell function or activation. In certain embodiments, the autoimmune disease is rheumatoid arthritis (RA), diabetes such as type 1 diabetes mellitus, psoriasis, psoriatic arthritis, ankylosing spondylitis, systemic sclerosis, multiple sclerosis, SLE, Sjögren's disease, Antiphospholipid syndrome, Pemphigus vulgaris, Spondylarthropathies, ulcerative colitis, uveitis, or Crohn's disease.

In certain embodiments, the chronic inflammatory disease includes cardiovascular, neurodegenerative diseases, diabetes, metabolic syndrome, periodontitis, and atherosclerosis.

In certain embodiments, the IGSF8 polypeptide comprises a full-length, an ECD, or a soluble fragment of IGSF8, which inhibits NK and/or T cell proliferation, viability, and/or function. In some embodiments, the ECD of IGSF8 comprises, consists essentially of, or consists of an Fc fusion of the ECD, such as an Fc fusion of the D1 (or Ig-V set) domain of IGSF8 that binds to KIR3DL1/2. In some embodiments, the Fc is a human IgG1 Fc fusion, human IgG2 Fc fusion, human IgG3 Fc fusion, or human IgG4 Fc fusion. In some embodiments, the Fc is a human IgG1 Fc fusion. The fusion may be at the C-terminus of IGSF8 or fragment thereof.

In some embodiments, the IGSF8 antagonist may be a small molecule or a peptide, e.g., a small peptide. In some embodiments, the IGSF8 antagonist may be a small peptide comprising an amino acid sequence of an IGSF8 ECD fragment. In some embodiments, the IGSF8 antagonist may be a small peptide comprising residues S165-M186 of KIR3DL1/2. In some embodiments, the IGSF8 antagonist is a small peptide having, e.g., from 5-50, from 3 to 20, e.g., 3 to 15 or 3 to 10 amino acids, which peptide may be linear or circular, with a sequence comprising an IGSF8 fragment, an IGSF8 ECD fragment, or a variant of an IGSF8 fragment, or IGSF8 ECD fragment. Such a variant of a IGSF8 may have, for example, at least 95%, at least 97%, at least 99% sequence identity to the native fragment sequence from which it is derived. In certain embodiments, the IGSF8-derived antagonists (such as IGSF8 ECD fragments or derivatives thereof) retains the ability to bind to KIR3DL1/2 without triggering the inhibitory function of IGSF8 on KIR3DL1/2, such that the antagonists function like dominant negative inhibitors of IGSF8-mediated KIR3DL1/2 function.

In certain embodiments, any of the polypeptides of the invention, including antibodies antigen-binding portion thereof, IGSF8 polypeptide and ECD thereof, may have a heterologous signal peptide when synthesized. In order for some secreted proteins to express and secrete in large quantities, a signal peptide from a heterologous protein may be desirable. Employing heterologous signal peptides may be advantageous in that a resulting mature polypeptide may remain unaltered as the signal peptide is removed in the ER during the secretion process. The addition of a heterologous signal peptide may be required to express and secrete some proteins.

Non-limiting exemplary signal peptide sequences are described, e.g., in the online Signal Peptide Database maintained by the Department of Biochemistry, National University of Singapore. See Choo et al, BMC Bioinformatics, 6: 249 (2005); and PCT Publication No. WO 2006/081430.

### 12. KIR3DL1/2 ECDs, Fusions, and Small Peptides

In some embodiments, the KIR3DL1/2 antagonist is a KIR3DL1/2 polypeptide, such as a fragment of KIR3DL1/2 or a fragment of IGSF8 that inhibits binding of KIR3DL1/2 to IGSF8 (e.g, inhibits binding of KIR3DL1/2 to the D1 or Ig-V set domain of IGSF8).

In some embodiments, the KIR3DL1/2 fragment is a KIR3DL1/2 extracellular domain (ECD). In some embodiments, the invention provides KIR3DL1/2 fragment, for example, comprising at least 30%, 40%, 50%, 60%, 70%, 80%, 85%, 90%, or at least 95% of the full length KIR3DL1/2 ECD amino acid sequence from which it is derived. In some embodiments, the fragment comprises the middle (2^{nd}, or D2) Ig-like domain of KIR3DL1/2 that binds IGSF8.

In some embodiments, the KIR3DL1/2 fragment is a full-length KIR3DL1/2 ECD. In some embodiments, the KIR3DL1/2 fragment is a partial KIR3DL1/2 ECD that comprises the middle (2^{nd}, or D2) Ig-like domain that binds IGSF8. In some embodiments, the KIR3DL1/2 fragment comprises, consists essentially of, or consists of the middle (2^{nd}, or D2) Ig-like domain of KIR3DL1/2 that binds IGSF8. In some embodiments, the KIR3DL1/2 fragment comprises, consists essentially of, or consists of the 2^{nd} and the 3^{rd} (D2 and D3) Ig-like domain of KIR3DL1/2 that together bind IGSF8. In some embodiments, the KIR3DL1/2 fragment comprises, consists essentially of, or consists of a polypeptide or epitope comprising residues S165 and M186 of KIR3DL1/2 that binds IGSF8, and inhibits IGSF8 binding to KIR3DL1/2. In some embodiments, the polypeptide or epitope is about 25 residues, 30 residues, 35 residues, 40 residues, 45 residues, or about 50 residues. In some embodiments, the polypeptide or epitope independently comprises about 1-20, about 2-15, about 3-10, about 5-8, about 2-7, or about 3-5 residues of KIR3DL1/2 that is immediately N-terminal to S165, immediately C-terminal to M186, or both immediately N-terminal to S165 and immediately C-terminal to M186.

In some embodiments, the invention provides a KIR3DL1/2 ECD variant, for example, comprising at least 80%, at least 85%, at least 90%, at least 92%, at least 95%, at least 97%, at least 98%, or at least 99% sequence identity with the full length IGSF8 ECD or fragment (e.g., the Ig-V set D1 domain) from which it is derived. In some embodiments, the variant retains the ability to bind KIR3DL1/2.

In other embodiments, the KIR3DL1/2 ECD is from a non-human KIR3DL1/2 ECD and may be either full length, a fragment (e.g., the D2 or middle Ig-like domain), or a variant (e.g., one with at least 80%, at least 85%, at least 90%, at least 92%, at least 95%, at least 97%, at least 98%, or at least 99% sequence identity and retains the ability to bind IGSF8).

In a related embodiment, the invention provides a KIR3DL1/2 variant that lacks the first or D Ig-like C2 domain of the ECD of KIR3DL1/2, but retains the D2 Ig-like domain of the ECD. Such variant may substantially maintain the functions of wt KIR3DL1/2, such as the ability to bind IGSF8.

In some embodiments, the KIR3DL1/2 or fragment or variant is combined with at least one fusion partner.

Thus, in some such embodiments, the invention provides a fusion of full-length KIR3DL1/2, such as an ECD C-terminal fusion with an Ig Fc region. In one embodiment, the Ig Fc fusion is a human IgG1 Fc fusion.

In any of the fusion molecule embodiments above, the fusion partner may comprise an immunoglobulin Fc molecule, for example, a human Fc molecule (e.g., human IgG1 Fc). In other embodiments, the fusion partner may be a different molecule such as albumin or polyethylene glycol (PEG). In some embodiments, more than one fusion partner may be attached to the KIR3DL1/2 or ECD thereof (such as ECD fragment comprising the D2 domain that binds IGSF8). In some embodiments, the fusion partner (or partners) is attached at the C-terminal, while other attachments are also possible such as on an amino acid side-chain or at the N-terminus. The attachment of a fusion partner to KIR3DL1/2 or fragments (e.g., ECD or D2 of ECD) or variants may be direct (i.e. by a covalent bond) or indirect through a linker. A linker may comprise, for example, at least one intervening amino acid or some other chemical moiety serving to link the fusion partner to the ECD either covalently or noncovalently.

In any of the above embodiments, the KIR3DL1/2 polypeptide may either include a signal sequence or be in a mature form, i.e., not including a signal sequence. The signal sequence may be from a native KIR3DL1/2 molecule or it may be a signal sequence from a different protein, for example one chosen to enhance expression and/or secretion of the KIR3DL1/2 polypeptide/fragment in cell culture. In some embodiments, a protein tag may be included to facilitate enrichment or purification.

In any of the above cases, a KIR3DL1/2 ECD may be part of a fusion molecule such that the above amino acid sequence may be joined to a fusion partner either directly or via a linker, such as an Fe, albumin, or PEG. For example, in some embodiments the ECD fusion molecule may comprise one of the above sequences plus an immunoglobulin Fc sequences, or an Fc from human IgG1. An ECD Fc fusion molecule may be formed by a direct attachment of the KIR3DL1/2 ECD amino acid sequence to the Fe amino acid sequence or via a linker (either an intervening amino acid or amino acid sequence or another chemical moiety).

In some embodiments, the KIR3DL1/2 antagonist may be a small molecule or a peptide, e.g., a small peptide. In some embodiments, the KIR3DL1/2 antagonist may be a small peptide comprising an amino acid sequence of an IGSF8 ECD fragment that binds to the D2 domain of KIR3DL1/2 and inhibits IGSF8-KIR3DL1/2 interaction but does not trigger the inhibitory function of KIR3DL1/2 on NK cells (which can be assayed by IFNγ secretion by NK cells). In some embodiments, the KIR3DL1/2 antagonist is a small peptide having, e.g., from 5-50, from 3 to 20, e.g., 3 to 15 or 3 to 10 amino acids, which peptide may be linear or circular, with a sequence comprising an IGSF8 fragment, an IGSF8 ECD fragment, or a variant of an IGSF8 fragment, or IGSF8 ECD fragment, that inhibits IGSF8-KIR3DL1/2 binding. Such a variant of a IGSF8 may have, for example, at least 95%, at least 97%, at least 99% sequence identity to the native fragment sequence from which it is derived.

In certain embodiments, any of the polypeptides of the invention, including antibodies antigen-binding portion thereof, polypeptide and ECD thereof, may have a heterologous signal peptide when synthesized. In order for some secreted proteins to express and secrete in large quantities, a signal peptide from a heterologous protein may be desirable. Employing heterologous signal peptides may be advantageous in that a resulting mature polypeptide may remain unaltered as the signal peptide is removed in the ER during the secretion process. The addition of a heterologous signal peptide may be required to express and secrete some proteins.

Non-limiting exemplary signal peptide sequences are described, e.g., in the online Signal Peptide Database maintained by the Department of Biochemistry, National University of Singapore. See Choo et al, BMC Bioinformatics, 6: 249 (2005); and PCT Publication No. WO 2006/081430.

### 13. Co-Translational and Post-Translational Modifications

In some embodiments, a polypeptide such as IGSF8 and/or KIR3DL1/2 or ECD thereof is differentially modified during or after translation, for example by glycosylation, sialylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease; NABH4; acetylation; formylation; oxidation; reduction; and/or metabolic synthesis in the presence of tunicamycin.

Additional post-translational modifications encompassed by the invention include, for example, N-linked or O-linked carbohydrate chains; processing of N-terminal or C-terminal ends; attachment of chemical moieties to the amino acid backbone; chemical modifications of N-linked or O-linked carbohydrate chains; and addition or deletion of an N-terminal methionine residue as a result of prokaryotic host cell expression.

### 14. Nucleic Acid Molecules Encoding IGSF8 Antagonists and/or KIR3DL1/2 Antagonists

The invention also provides nucleic acid molecules comprising polynucleotides that encode one or more chains of an antibody described herein, such as IGSF8 antibody and/or KIR3DL1/2 antibody. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

Nucleic acids encoding other IGSF8 antagonists and/or KIR3DL1/2 antagonists are also provided, such as fragments or variants of IGSF8 including IGSF8 ECD molecules (e.g., the KIR3DL1/2-binding D1 Ig-V set domain), or IGSF8 ECD fusion molecules and including fragments or variants thereof, and fragments or variants of KIR3DL1/2 including KIR3DL1/2 ECD molecules (e.g., the middle or D2 IGSF8-binding Ig-like domain of KIR3DL1/2), or KIR3DL1/2 ECD fusion molecules and including fragments or variants thereof. Nucleic acid molecules may be constructed using recombinant DNA techniques conventional in the art. In some embodiments, a nucleic acid molecule is an expression vector that is suitable for expression in a selected host cell.

### 15. Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004). In some embodiments, a vector is chosen for in vivo expression of IGSF8 antagonist in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

### 16. Host Cells

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of IGSF8 antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

### 17. Assay, Diagnosis, Prognosis Methods

In a related aspect, the invention also provides an in vitro assay method for determining the ability of an anti-IGSF8 antagonist or anti-KIR3DL1/2 antagonist to inhibit IGSF8-KIR3DL1/2 binding, or a screening method for identifying an anti-IGSF8 antagonist or anti-KIR3DL1/2 antagonist (such as small molecule or peptide antagonist) to inhibit IGSF8-KIR3DL1/2 binding, the method comprising contacting a candidate anti-IGSF8 antagonist or candidate anti-KIR3DL1/2 antagonist (e.g., an antibody, a peptide fragment, or a small molecule) with an IGSF8 polypeptide and a KIR3DL1/2 polypeptide, wherein the IGSF8 polypeptide and/or the KIR3DL1/2 polypeptide are labeled by a detectable signal, and wherein inhibition of IGSF8-KIR3DL1/2 binding by the candidate anti-IGSF8 antagonist or anti-KIR3DL1/2 antagonist leads to a detectable or measurable change in the detectable signal.

In a related aspect, the invention also provides an in vitro assay method for determining the ability of an anti-IGSF8 antagonist or an anti-KLRC1/D1 antagonist, to inhibit IGSF8-KLRC1/D1 binding, or a screening method for identifying an anti-IGSF8 antagonist or anti-KLRC1/D1 antagonist (such as small molecule or peptide antagonist) to inhibit IGSF8-KLRC1/D1 binding, the method comprising contacting a candidate anti-IGSF8 antagonist or a candidate anti-KLRC1/D1 antagonist (e.g., an antibody, a peptide fragment, or a small molecule) with an IGSF8 polypeptide and a KLRC1/D1 polypeptide, wherein the IGSF8 polypeptide and/or the KLRC1/D1 polypeptide are labeled by a detectable signal, and wherein inhibition of IGSF8-KLRC1/D1 binding by the candidate anti-IGSF8 antagonist or anti-KLRC1/D1 antagonist leads to a detectable or measurable change in the detectable signal.

In one embodiment, the IGSF8 polypeptide comprises the D1 or Ig-V set domain of IGSF8 responsible for KIR3DL1/2 binding, and the KIR3DL1/2 polypeptide comprises the D2 (or the middle) Ig-like domain of KIR3DL1/2.

In one embodiment, the IGSF8 polypeptide is immobilized on a solid support, or is expressed on a cell, such as a cell that does not express MHC Class I (HLA) receptor. An exemplary cell is K562 that stably or inducibly expresses exogenous IGSF8, which can be transduced into K562 cells by a vector, such as a lentiviral vector encoding the IGSF8 polypeptide. In another embodiment, the cell is CT26 cell (ATCC CRL-2638™ Mus musculus colon carcinoma) expressing exogenous IGSF8.

In one embodiment, the KIR3DL1/2 polypeptide is labeled by a detectable signal, such as biotin. The biotin label can be detected by a streptavidin linked signal, such as PE-streptavidin.

In an alternative embodiment, the IGSF8 polypeptide and the KIR3DL1/2 polypeptide can be labeled by a fluorescent molecule and a molecule that suppresses fluorescent emission when the fluorescent molecule and the suppressor are in close proximity to each other, but a fluorescent signal is generated once the antagonist inhibits IGSF8-KIR3DL1/2 binding.

In a related aspect, the invention provides a method of detecting the presence or level of an IGSF8 polypeptide in a sample, the method comprising contacting the IGSF8 polypeptide in the sample with the antibody, monoclonal antibody, or antigen-binding portion/fragment thereof, of the invention, wherein said antibody, monoclonal antibody, or antigen-binding portion/fragment thereof is labeled by a detectable label, or can be attached to a detectable label.

In certain embodiments, said antibody, monoclonal antibody, or antigen binding portion/fragment thereof, forms a complex with the IGSF8 polypeptide, and the complex is detected in the form of an enzyme linked immunosorbent assay (ELISA), radioimmune assay (RIA), immunochemical method, Western blot, or an intracellular flow assay.

In a related aspect, the invention provides a method for monitoring the progression of a disorder associated with aberrant (e.g., higher than normal) IGSF8 expression in a subject, the method comprising: a) detecting, in a sample obtained from the subject, at a first point in time a first level of IGSF8 using the antibody, monoclonal antibody, or antigen-binding portion/fragment thereof, of the invention; b) repeating step a) at a subsequent point in time to obtain a second level of IGSF8; and c) comparing the first and the second levels of IGSF8 detected in steps a) and b), respectively, to monitor the progression of the disorder in the subject, wherein a higher second level than the first level is indicative that the disease has progressed.

In certain embodiments, between the first point in time and the subsequent point in time, the subject has undergone a treatment to ameliorate the disorder.

In a related aspect, the invention provides a method for predicting the clinical outcome of a subject afflicted with a disorder associated with aberrant (e.g., higher than normal) IGSF8 expression, the method comprising: a) determining the level of IGSF8 in a first sample obtained from the subject, using the antibody, monoclonal antibody, or antigen-binding portion/fragment thereof, of the invention; b) determining the level of IGSF8 in a second sample obtained from a control subject having a good clinical outcome, using the antibody, monoclonal antibody, or antigen-binding portion/fragment thereof, of the invention; and c) comparing the level of IGSF8 in the first and the second samples; wherein a significantly higher (e.g., >20%, >50% or more increase) level of IGSF8 in the first sample as compared to the level of IGSF8 in the second sample is an indication that the subject has a worse clinical outcome, and/or, wherein a significantly lower (e.g., >20%, >50% or more decrease) level of IGSF8 in the first sample as compared to the level of IGSF8 in the second sample is an indication that the subject has a better clinical outcome.

In a related aspect, the invention provides a method of assessing the efficacy of a therapy for a disorder associated with aberrant (e.g., higher than normal) IGSF8 expression in a subject, the method comprising: a) determining the level of IGSF8 using the antibody, monoclonal antibody, or antigen-binding portion/fragment thereof, of the invention, in a first sample obtained from the subject prior to providing at least a portion of the therapy to the subject, and b) repeat step a) in a second sample obtained from the subject following provision of said portion of the therapy, wherein a significantly lower (>20%, >50% or more decrease) level of IGSF8 in the second sample, relative to the first sample, is an indication that the therapy is efficacious for inhibiting the disorder in the subject; and/or, wherein a substantially identical or higher level of IGSF8 in the second sample, relative to the first sample, is an indication that the therapy is not efficacious for inhibiting the disorder in the subject.

In certain embodiments, the disease is cancer.

In a related aspect, the invention provides a method of assessing the efficacy of a test compound for inhibiting a disorder associated with aberrant (e.g., higher than normal) IGSF8 expression in a subject, the method comprising: a) determining the level of IGSF8 using the antibody, monoclonal antibody, or antigen-binding portion/fragment thereof, of the invention, in a first sample obtained from the subject, wherein the first sample has been exposed to an amount of the test compound; and b) determining the level of IGSF8 using the antibody, monoclonal antibody, or antigen-binding portion/fragment thereof, of the invention, in a second sample obtained from the subject, wherein the second sample has not been exposed to the test compound, wherein a significantly lower (>20%, >50% or more decrease) level of IGSF8 in the first sample relative to that of the second sample, is an indication that the amount of the test compound is efficacious for inhibiting the disorder in the subject, and/or, wherein a substantially identical level of IGSF8 in the first sample relative to that of the second sample, is an indication that the amount of the test compound is not efficacious for inhibiting the disorder in the subject.

In certain embodiments, the first and second samples are portions of a single sample obtained from the subject or portions of pooled samples obtained from the subject.

In certain embodiments, the disorder is a cancer.

In certain embodiments, the cancer is lung cancer, renal cancer, pancreatic cancer, colorectal cancer, Acute myeloid leukemia (AML), head and neck carcinoma, liver cancer, ovarian cancer, prostate cancer, uterine cancer, gliomas, glioblastoma, neuroblastoma, breast cancer, pancreatic ductal carcinoma, thymoma, B-CLL, leukemia, B cell lymphoma, and a cancer infiltrated with immune cells (e.g., T cells and/or NK cells) expressing a receptor to IGSF8 (e.g., KIR3DL1, KIR3DL2, and/or KLRC1/D1).

In certain embodiments, the sample comprises cells, serum, peritumoral tissue, and/or intratumoral tissue obtained from the subject.

In certain embodiments, the subject is a human.

In a related aspect, the invention provides a method of screening for a functional IGSF8 antagonist, the method comprising contacting a candidate agent (e.g., small molecule, peptide, aptamer, polynucleotide, etc) with a co-culture of NK cells and target cells that express IGSF8 and are resistant to NK cell-mediated cytotoxicity, and identifying the candidate agent that promotes NK cell-mediated cytolytic activity towards the target cell, thereby identifying the candidate agent as an IGSF8 antagonist.

In a related aspect, the invention provides a method of screening for a functional IGSF8 antagonist, the method comprising contacting a candidate agent (e.g., small molecule, peptide, aptamer, polynucleotide, etc) with a Jurkat NFAT reporter cell in the presence of T-cell activation signals and IGSF8, wherein the candidate agent is identified as the functional IGSF8 antagonist, when the reporter cell is not activated in the absence of the candidate agent and is activated in the presence of the candidate agent.

In a related aspect, the invention provides an antibody which specifically bind KIR3DL1/2 for use in a method of treating cancer, through inhibiting KIR3DL1/2-IGSF8 interaction, thereby stimulating NK cell activation.

In a related aspect, the invention provides an antibody which specifically bind KIR3DL1/2 for use in a method of treating cancer, preferably through combination with a second therapeutic agent of the invention as described herein.

### EXAMPLES

### Example 1 Loss of IGSF8 in Colo205 Cancer Cells Enhances Natural Killer (NK) Cell Cytotoxicity Against Colo205 Cells

This experiment demonstrates that IGSF8 activity/expression negatively regulates NK cell cytotoxicity towards cancer cells (e.g., Colo205 colorectal cancer cells), and loss of IGSF8 activity/expression enhances NK cell cytotoxicity.

A genome-wide co-culture screen using NK cell and Colo205 cancer cells were conducted to determine which gene(s) are required or are essential for Colo205 cancer cells to evade killing by NK cells. In particular, Colo205 tumor cells were transduced with a whole-genome guide RNA (gRNA) Cas9 library and then subjected to two successive rounds of overnight co-culture with primary human NK cells which exhibited a typical activated phenotype. The resulting population of cells were sequenced to identify depleted gRNA that sensitized tumor cells to killing by NK cells. Model-based Analysis of Genome-wide CRISPR/Cas9 Knockout (MAGeCK) software was subsequently used to count the reads and perform gene/gRNA fold change, selection score and statistical analyses between treated and untreated (control) samples.

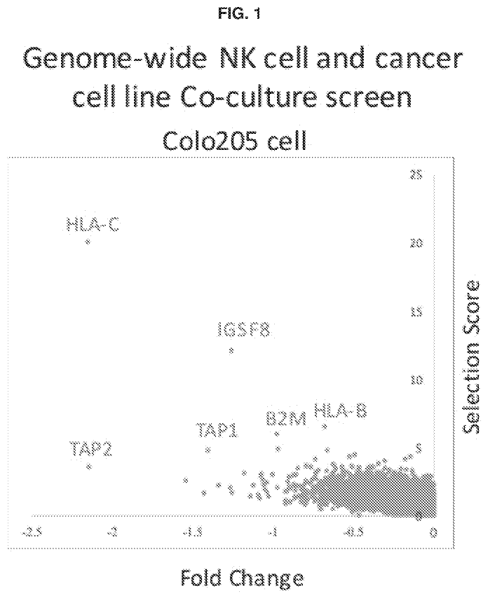

A volcano dot plot encompassing selection score and gRNA fold change was generated for each gene tested in the assay, showing the top depleted genes after co-culturing with NK cells. The genes associated with antigen presentation (such as HLA-C, Tap1, Tap2, and B2m), when depleted, were found to render the tumor cells most sensitive to killing by NK cells. Additionally, IGSF8 was one of the two top hits, the loss of which activity/expression in Colo205 cell enhanced NK cell cytotoxicity. The results were summarized in FIG. 1.

### Example 2 IGSF8 Reduced Viability of Primary Natural Killer Cells and Primary T Cells from Healthy Donors

To further demonstrate the negative impact of IGSF8 on NK cell activity, increasing concentrations of recombinant human IGSF8 tagged by a human Fc region (IGSF8-hFc) was incubated with primary human NK cells isolated from two healthy donors, and the viability of these primary NK cells over IGSF8-hFc concentrations (dose response curve) was determined.

The primary NK or T cells were isolated from healthy donors' peripheral blood mononuclear cells (PBMCs) using commercial negative/positive isolation kits (StemCell Technologies, Inc.). NK or T cells were cultured in RPMI medium supplemented with 10% Fetal Bovine Serum (FBS), penicillin/streptomycin, L-glutamine, non-essential amino acids, sodium pyruvate, HEPES, 2-Mercaptoethanol and recombinant human IL-2 (1,000 IU/mL), and were incubated at 37° C. with 5% CO₂. T cells were activated by anti-CD3 and anti-CD28 beads once a week.

The primary NK or T cells were then seeded in 96-well plates (3,000 cells per well) and cultured 18 to 24 hours before adding the IGSF8-hFc fusion protein or human Fc protein as negative control. Cell viability was determined by Cell Counting Kit 8 (CCK8) method with three biological replicates after 72 hours.

Data in FIG. 2A shows that NK cell viability was reduced in vitro as concentration of IGSF8-hFc increased. Meanwhile, a human Fc used as a control in the same assay did not substantially affect NK cell viability. This data is consistent with the observation in Example 1 that the presence of IGSF8 on Colo205 cancer cells inhibited NK cell function, possibly at least partially through reducing NK cell viability.

Similar results were also obtained for primary T lymphocytes isolated from Donor 2. See FIG. 2B.

These data showed that IGSF8 reduced viability of both primary NK cells and primary T cells in vitro, suggesting a mechanism by which antagonizing IGSF8 activity can be used to restore or promote NK/T cell activity. Meanwhile, IGSF8 (including hFc fusion thereof) may be used to inhibit T- and/or NK cell activity in disease treatment, where excessive T cell and/or NK cell activity is detrimental, such as in certain autoimmune diseases or graft-vs-host diseases.

### Example 3 CRISPR/Cas9-Mediated IGSF8 Knock-Out in B16-F10 Tumor Cells Retards Tumor Growth In Vivo in Syngeneic Tumor Model

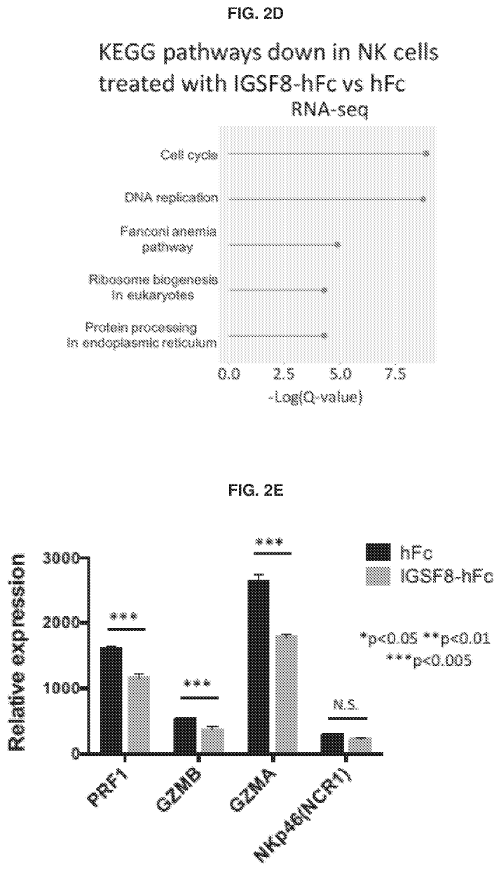

To further demonstrate the negative impact of tumor-expressed IGSF8 on the host immune system, B16-F10 melanoma cells with or without IGSF8 function/expression (IGSF8 null) were compared in their ability to grow as syngeneic tumors in wild-type (WT) mice. The IGSF8 gene was deleted/inactivated by the CRISPR/Cas9-mediated gene editing using IGSF8-specific single guide RNA (sgRNA) sequences. Two separate lines of IGSF8-inactivated B16-F10 cancer cell lines were established, namely sg IGSF8-1 and sg IGSF8-2, with different regions of IGSF8 being targeted. Down-regulation of IGSF8 expression was verified by flow cytometry (data not shown). As a negative control, the adeno associated virus integration sequence AAVS1 was also similarly deleted/inactivated by CRISPR/Cas9-mediated gene editing in B16-F10 cells (sg AAVS1). Then one million each of unaltered B16-F10 cancer cells, sg IGSF8-1 cells, sg IGSF8-2 cells, and sg AAVS1 cells, respectively, were implanted into C57BL/6 mice (8 mice per group) at Day 0, and tumor volumes in each mouse was measured and calculated according to standard methods over 2 weeks. The results were averaged for each group with standard deviation, and plotted in FIG. 3A.

It is apparent that the absence of IGSF8 expression/function significantly retarded tumor growth as early as Day 11 (p<0.05), and the difference in tumor volume was significant at Day 14 (p<0.0001). This in vivo result is consistent with the previous observation that IGSF8 reduced NK and T cell viability in vitro.

Interestingly, the presence or absence of IGSF8 was apparently not required for tumor growth per se. Relative tumor cell growth rates over a course of 6 days, as measured in vitro for each of the above test cell lines, were essentially indistinguishable (see FIG. 3B).

This result is also consistent with the observation that the average essential score of IGSF8, in a genome-wide CRISPR screen based on 625 types of cancer cell lines (Data downloaded from DepMap Portal), was just slightly negative and very close to 0 (about −0.05) (data not shown), suggesting that IGSF8 plays a very minor (if any) direct role in cell growth. In contrast, prototypical oncogenes such as myc, and cell cycle genes such as CDK1, were both well below −1.0, while tumor suppressor gene Tp53 has a +0.2 average essential score (data not shown).

Together, these data strongly suggest that the absence of IGSF8 on tumor cells retarded tumor cell growth in vivo, not through reducing the growth rate of the tumor cells per se, but likely through negatively affecting (e.g., inhibiting) the host immune system.

### Example 4 TNFα Signaling Pathway is Negatively Regulated by IGSF8

To identify the mechanism by which loss of IGSF8 in tumor cells allows the tumor cells to escape immune surveillance, RNA-sequencing was performed for both IGSF8-null and AAVS1-control B16-F10 melanoma cells as described in Example 3.

Importantly, it was found that depletion of IGSF8 in B16-F10 cells activated TNFα signaling pathway, and increased gene expressions of many immune-related cytokines (especially, CXCL10 and CXCL9, see FIGS. 5A-5B). CXCL10 is a small cytokine belonging to the CXC chemokine family, which plays role to induce chemotaxis, promote differentiation, and multiplication of leukocytes, and cause tissue extravasation. CXCL10 is secreted by several cell types in response to IFN-7.

As CXCL9 and CXCL10 were known to regulate immune cell migration, differentiation, and activation, leading to tumor suppression (Tokunaga et al., Cancer Treat Rev. 63:40-47, 2018), the effect of IGSF8 on CXCL10 expression in other human cancer cells was examined.

Specifically, IGSF8 was knocked out in six different human cancer cell lines by CRISPR/Cas9, and RNA-sequencing was performed for these IGSF8-null and AAVS1-control human cancer cells. FIG. 4 shows that relative expression of CXCL10 in the various tested tumor cell lines were increased, sometimes dramatically increased by almost 10-fold, in IGSF8 null cancer cells compared to the counterpart cancer cell lines with intact IGSF8. The tested cancer cell lines included: H292 (NCI-H292) is a human mucoepidermoid pulmonary carcinoma cell line; A549 is a human lung carcinoma cell line; Colo205 is a Dukes' type D, colorectal adenocarcinoma cell line; N87 is a human gastric carcinoma cell line; and A375 is a another human melanoma cell line.

These data suggest that IGSF8 may be a universal negative regulator of CXCL10 expression in various cancers, and deletion or inactivation of IGSF8 promotes CXCL10 expression.

### Example 5 Loss of IGSF8 Reprogramed the Tumor Microenvironment (TME) to Improve NK and T Cell Activities

To identify the mechanism by which inactivation of IGSF8 in B16-F10 tumors significantly decreased tumor growth (see FIG. 3A), IGSF8-null and AAVS1-control B16-F10 cells were subcutaneously inoculated into C57BL6 mice. When the tumors grew to about 1 to 2 mm³, the tumors were isolated, and RNA-sequencing was performed on isolated tumors.

It was found that the genes (Gzmb, Prfl, etc.) representing the immune cytolytic activity (CYT) of tumors were significantly up-regulated in IGSF8-null tumors (FIG. 5B), but not in IGSF8-null cells (FIG. 5A). Moreover, CD8 gene (CD8a and CD8b) expression in IGSF8-null tumors (but not in IGSF8 null cells, FIG. 5A) were also dramatically increased (FIG. 5B), indicating more CD8⁺ T cell infiltration into IGSF8-null tumors.

These data suggest that depletion of IGSF8 in B16-F10 tumors reprogramed the Tumor Microenvironment (TME) to improve immune cytolytic activity in vivo for tumor suppression, possibly by increasing CD8⁺ T cell infiltration.

More importantly, loss of IGSF8 increased the expression of well established IO targets (PDCD1, CD274, LAG3, TIM3 or TIGIT) (FIG. 5D), indicating that combining IGSF8 antagonists with antagonists of PDCD1, CD274, Lag3, TIM3 or TIGIT in a combination therapy is effective for cancer treatment. See below.

### Example 6 IGSF8 was Overexpressed in Many Cancer Types and Resulted in Worse Clinical Outcome

This example demonstrates that IGSF8 is overexpressed by a number of cancer cells, possibly as a mechanism to evade host immune response.

FIG. 6A shows gene expression of IGSF8 in a number of human cancer cell lines based on data from Broad Institute Cancer Cell Line Encyclopedia (CCLE). Top 30 cancer cell lines with the highest IGSF8 expression in the CCLE dataset are listed below.

In addition, based on analysis of The Cancer Genome Atlas (TCGA) Datasets, IGSF8 was found to be significantly overexpressed in many types of cancers: BLCA: Bladder Cancer, BRCA: Breast Cancer, HNSC: Head-Neck Squamous Cell Carcinoma, LUAD: Lung Adenocarcinoma, LUSC: Lung Squamous Cell Carcinoma, PRAD: Prostate Adenocarcinoma, SKCM: Skin Cutaneous Melanoma, THCA: Thyroid Cancer, UCEC: Uterine Corpus Endometrial Carcinoma, READ: Rectum Adenocarcinoma, COAD: Colon Adenocarcinoma (FIG. 6B).

RSEM (RNA-Seq by Expectation-Maximization)

| | XBH25 expression |
|---|---|
| Cell line | (log2(RSEM) |
| MALME3M_SKIN | 9.226186 |
| HS936T_SKIN | 8.806057 |
| IGR37_SKIN | 8.626165 |
| K029AX_SKIN | 8.458715 |
| COLO679_SKIN | 8.448694 |
| DU4475_BREAST | 8.439735 |
| MELHO_SKIN | 8.34886 |
| COLO741_SKIN | 8.26553 |
| TT_THYROID | 8.093418 |
| SKMEL2_SKIN | 8.006397 |
| SKMEL5_SKIN | 8.005364 |
| G361_SKIN | 7.911904 |
| NCIH520_LUNG | 7.905627 |
| C32_SKIN | 7.901319 |
| COLO829_SKIN | 7.896537 |
| MHHNB11_AUTONOMIC_GANGLIA | 7.838727 |
| UACC257_SKIN | 7.74993 |
| HS939T_SKIN | 7.722027 |
| UBLC1_URINARY_TRACT | 7.69668 |
| KURAMOCHI_OVARY | 7.67295 |
| OE19_OESOPHAGUS | 7.598727 |
| UACC62_SKIN | 7.554536 |
| CAL148_BREAST | 7.51395 |
| HCC1419_BREAST | 7.477927 |
| JHH2_LIVER | 7.471425 |
| HS944T_SKIN | 7.460963 |
| SKMEL30_SKIN | 7.453764 |
| AU565_BREAST | 7.443264 |
| SKMEL24_SKIN | 7.425736 |
| BT483_BREAST | 7.419773 |

The clinical relevancy of IGSF8 expression was also demonstrated by data based on The Cancer Genome Atlas (TCGA). Specifically, FIG. 6C shows that higher expression of IGSF8 is associated with worse clinical outcome in different cancer types. For example, in melanoma, the 13 patients with high IGSF8 expression (“Top”) had a much worse survival curve than that for the 304 patients with lower IGSF8 expression (“Bottom”). The difference is statistically significant (p<0.0018).

The same has been observed in cervical cancer, LUAD (lung adenocarcinoma), lymphoma (including diffused large B cell lymphoma or DLBCL), LUSC (Lung Squamous Cell Carcinoma), READ (Rectum Adenocarcinoma), COAD (colon adenocarcinoma), and leukemia (including CLL).

Thus it is expected that IGSF8 antagonists of the invention, such as anti-IGSF8 antibodies or antigen-binding fragments thereof, are able to treat cancers with IGSF8 overexpression, such as the cancers listed in the table above and those in FIGS. 6A-6C.

### Example 7 Anti-IGSF8 Antibodies Exhibit Nanomolar (nM) Affinity for IGSF8 Extracellular Domain (ED)

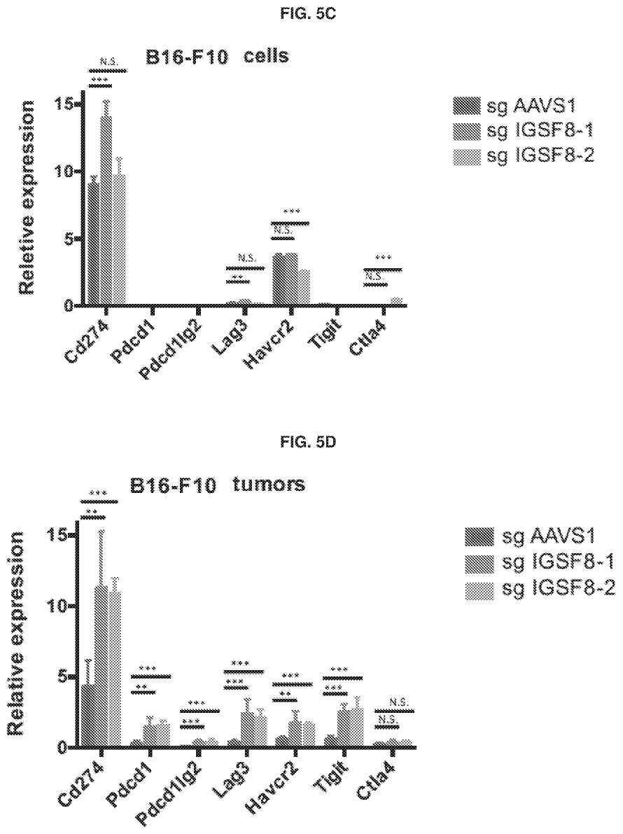

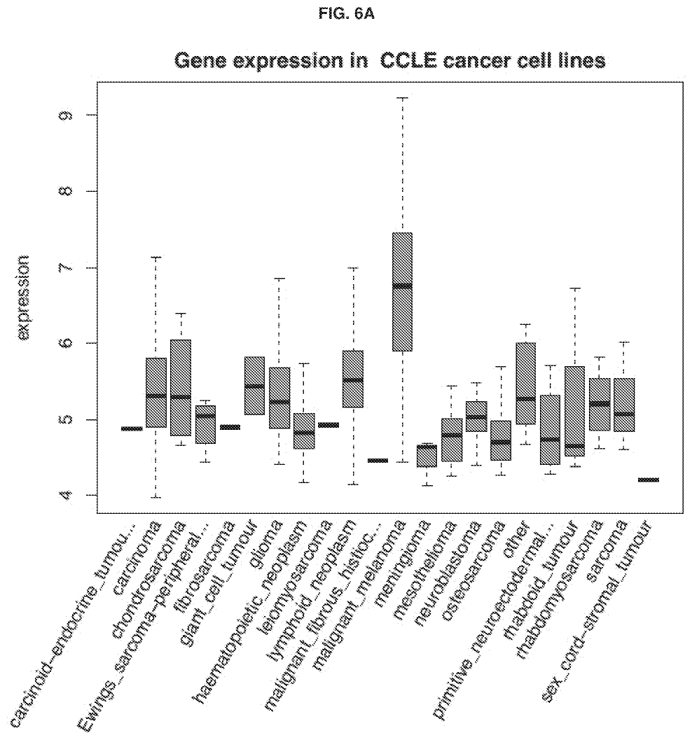

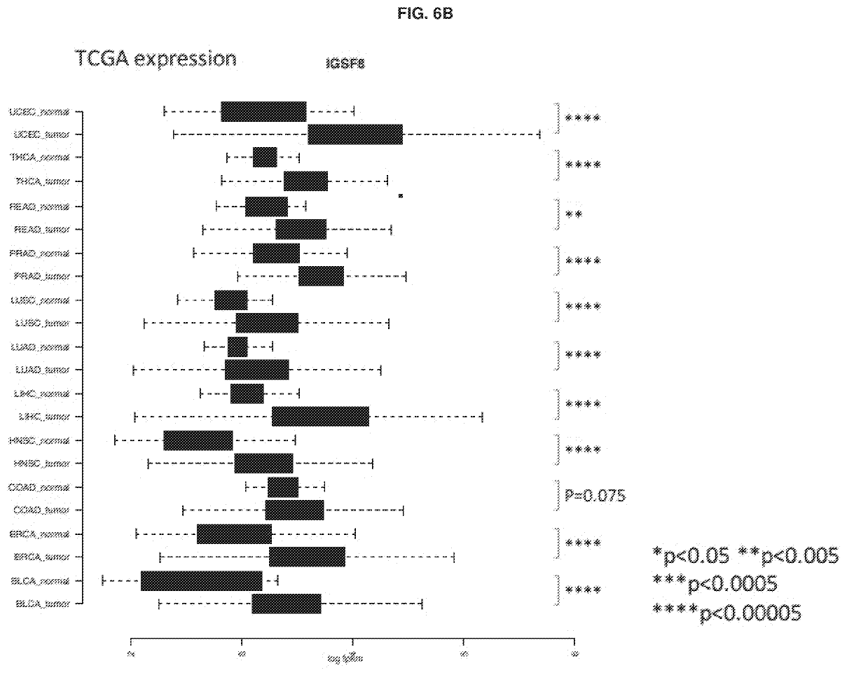

About 50 anti-IGSF8 monoclonal antibodies were produced, twelve of which, anti-IGSF8 C1 to C12, were tested in affinity binding assays using ELISA, all exhibited high affinity for the extracellular domain (ED) of IGSF8. See FIG. 7. The antibodies showing the strongest binding affinity have EC50 values of about mid- to low-nM range. See C1-C4, C8, and C11.

The sequences of these representative antibodies, including the light chain (LC) and heavy chain (HC) variable regions, the CDR regions, the framework regions (FR), and constant regions, are listed in the table below (H=heavy chain; L=light chain; CDR-H1 to -H3: the three heavy chain CDR sequences; CDR-L1 to -L3: the three light chain CDR sequences; FR: framework region).

In the antibody sequences below, HCVR and LCVR sequences are indictated, with the 6 CDR sequences double underlined. The rest of the sequences are heavy and light chain framework regions HFR1-HFR4 and LFR1 to LFR4. Thus each table contains the following 16 sequences in this order: CDR-H1, CDR-H2, CDR-H3, CDR-L1, CDR-L2, CDR-L3, HFR1-HFR4, LFR1-LFR4, HCVR and LCVR (i.e., SEQ ID NOs: 1-16 for antibody C1, SEQ ID NOs: 17-32 for antibody C2, etc.). For simplicity, only the HCVR and LCVR sequences with double underlined CDR sequences are shown.

| | Antibody C1 (SEQ ID NOs: 1-16) |
|---|---|
| HCVR | QVQLLQSGGGLVQPGGSLRLSCAASGFTFSRYRMSWVRQAPGKGLEWVSRI |
| | SRSGGATAYADSVKGRFTISRDNSKNTLYLQMNSLRAEDTAVYYCARDAT |
| | GRHYNGMDVWGRGTLVTVSS |
| LCVR | EIALTQSPSSLSASVGDRVTITCRASQTITRHLNWFQQKPGKAPNLLIHGTSA |
| | LQTGVPPRFSGGGSGTDFTLTINSLQPEDFGTYYCQQSHTKPWTFGPGTKVEI |
| | KRTV |
| | Antibody C2 (SEQ ID NOs: 17-32) |
| HCVR | EVQLLQSGGGLVQPGGSLRLSCAASGFTFSSYPMNWVRQAPGKGLEWVSRI |
| | SRSGGRTSYADSVKGRFTISRDNSKNTLYLQMNSLRAEDTAVYYCARDATR |
| | RHYNGMDVWGKGTTVTVSS |
| LCVR | DVVMTQSPATLSLSPGERASLSCRASRSVGKYLAWYQQKPGQAPRLLFYYA |
| | SLRAGDIPSRFTASGSGTDFTLTISRLEPEDFAVYYCQQYGSSPRTFGQGTKL |
| | EMKRTV |
| | Antibody C3 (SEQ ID NOs: 33-48) |
| HCVR | QVQLVESGGGLVQPGGSLRLSCAASGFTFSHYPMRWVRQAPGKGLEWVSSI |
| | RRSGGRTKYADSVKGRFTISRDNSKNTLYLQMNSLRAEDTAVYYCARDAT |
| | GRHYNGMDVWGKGTTVTVSS |
| LCVR | DVVMTQSPSSLSASVGDRVTITCRTSQVIGTSLNWYQQKPGRAPRLLIYSAS |
| | NLQSGVPSRFSGSGHGTQFTLTISSLQPEDFATYSCQQSSRVPHTFGQGTKLE |
| | MRRTV |
| | Antibody C4 (SEQ ID NOs: 49-64) |
| HCVR | QVQLLQSGGGLVQPGGSLRLSCAASGFTFSRYRMGWVRQAPGKGLEWVSS |
| | IARSGGRTYYADSVKGRFTISRDNSKNTLYLQMNSLRAEDTAVYYCARGVR |
| | YCSSPSCSRGPRYAMDVWGKGTTVTVSS |
| LCVR | EIVMTQSPSSVSASVGDRVTITCRASQGISSWLAWYQQKPGKAPKLLIYAAS |
| | SLQSGVPSRFSGSGSGTDFTLTISSLQPEDFATYYCQQANSFPITFGQGTRLEI |
| | KRTV |
| | Antibody C5 (SEQ ID NOs: 65-80) |
| HCVR | EVQLVQSGGGLVQPGGSLRLSCAASGFTFSRYRMAWVRQAPGKGLEWVSN |
| | ITRSGGVTRYADSVKGRFTISRDNSKNTLYLQMNSLRAEDTAVYYCARDPN |
| | RVTAISSHYGMDVWGKGTTVTVSS |
| LCVR | EIVLTQSPSTLSASVGDRVTISCRASQSISRWLAWYQQKPGQAPRLLIYDASN |
| | RATGVPARFSVSGSETDSTLTISSLEPEDFAMYYCQQRSNWPPMYTFGQGTK |
| | LEIKRTV |
| | Antibody C6 (SEQ ID NOs: 81-96) |
| HCVR | EVQLVESGGGLVQPGGSLRLSCAASGFTFSPYRMHWVRQAPGKGLEWVSRI |
| | NPSGGRTWYADSVKGRFTISRDNSKNTLYLQMNSLRAEDTAVYYCARDAT |
| | GRHYNGMDVWGQGTMVTVSS |
| LCVR | DIQMTQSPSTLSASVGDRVTITCRASQSINKWLAWYQQKPGKAPKLLIYKAS |
| | TLESGVPSRFSGSGSGTDFTLTINSLQPEDFATYYCQQSHSAPWTFGQGTKV |
| | EIERTV |
| | Antibody C7 (SEQ ID NOs: 97-112) |
| HCVR | EVQLEESGGGLVQPGGSLRLSCAASGFTFSSYPMNWVRQAPGKGLEWVSRI |
| | SRSGGRTSYADSVKGRFTISRDNSKNTLYLQMNSLRAEDTAVYYCARDATR |
| | RHYNGMDVWGKGTTVTVSS |
| LCVR | ETTLTQSPATLSLSPGERASLSCRASRSVGKYLAWYQQKPGQAPRLLFYYAS |
| | LRAGDIPSRFTASGSGTDFTLTISRLEPEDFAVYYCQQYGSSPRTFGQGTKLE |
| | MKRTV |
| | Antibody C8 (SEQ ID NOs: 113-128) |
| HCVR | EVQLQESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCARPYNS |
| | AWESYYYGMDVWGKGTTVTVSS |
| LCVR | DIQMTQSPSSVSASVGDRVTITCRASQGISSRLAWYQQKPGKAPKLLIYAAS |
| | SLQSGVPSRFSGSGSGTDFTLTISSLQPEDFATYYCQQRHSYPITFGQGTRLEI |
| | KRTV |
| | Antibody C9 (SEQ ID NOs: 129-144) |
| HCVR | EVQLVESGGGLVQPGGSLRLSCAASGFTFSRYDMSWVRQAPGKGLEWVSRI |
| | RYSGGRTGYADSVKGRFTISRDNSKNTLYLQMNSLRAEDTAVYYCARGVR |
| | YCSSPSCSRGPRYAMDVWGKGTTVTVSS |
| LCVR | DVVMTQSPATLSLSPGEGATLSCRASQSVRGYLAWYQQKPGQAPRLLIYDT |
| | FKRATGIPARFSGSGSGADFTLTISSLEPEDSAVYYCQQYFASPWTFGQGTK |
| | VEIKRTV |
| | Antibody C10 (SEQ ID NOs: 145-160) |
| HCVR | EVQLLESGGGLVQPGGSLRLSCAASGFTFSRYRMYWVRQAPGKGLEWVSTI |
| | SRSGGRTVYADSVKGRFTISRDNSKNTLYLQMNSLRAEDTAVYYCARDAT |
| | GRHYNGMDVWGQGTLVTV |
| LCVR | ETTLTQSPATLSVSPGERATLSCRASQSVSSNVAWYQQKPGQAPRLLMFGS |
| | GTRATGIPARFSGSGSGTEFTLTISSLQSEDFAAYYCQQYNDWPSFGQGTRV |
| | EIKGTV |
| | Antibody C11 (SEQ ID NOs: 161-176) |
| HCVR | EVQLVESGGGLVQPGGSLRLSCAASGFTFSRYRMYWVRQAPGKGLEWVSSI |
| | SSSGGRTKYADSVKGRFTISRDNSKNTLYLQMNSLRAEDTAVYYCARGVRY |
| | CSSPSCSRGPRYAMDVWGKGTTVTVSS |
| LCVR | DIQMTQSPSSLSASVGDRVTITCRASYVIRNDLSWYQQKPGKAPKLLIYGTSS |
| | LHNGVPSRFSGSGYGTYFTLTISSLQPEDFGTYYCLQDDKYPLTFGGGTKVEI |
| | KRTV |
| | Antibody C12 (SEQ ID NOs: 177-192) |
| HCVR | EVQLVESGGGLVQPGGSLRLSCAASGFTFSKYKMSWVRQAPGKGLEWVSTI |
| | APSGGGTRYADSVKGRFTISRDNSKNTLYLQMNSLRAEDTAVYYCARGGH |
| | FSNPWGQGTLVTVSS |
| LCVR | DVVMTQSPLSLPVTLGQPASISCRSSQSLVHTDGDTYLNWYQQRPGQSPRRL |
| | IYKVSKRDSGVPDRFSGSGSGTDFTLKISRVEAEDVGVYYCMQGIKRPYTLG |
| | QGTKLEIKRTV |
| | Antibody C13 (SEQ ID NOs: 193-208) |
| HCVR | EVQLVESGGGLVQPGGSLRLSCAASGFTFSPYRMHWVRQAPGKGLEWVSSI |
| | NRSGGRTNYADSVKGRFTISRDNSKNTLYLQMNSLRAEDTAVYYCATGRGI |
| | GTFRNWGQGTLVTVSS |
| LCVR | DIALTQSPATLSLSPGERATLSCRASQSVSTYLAWYQQKPGQAPRLLISDASN |
| | RATGIPARFSGSGSGTDFTLTISSLEPEDFAVYYCQQRNNWPPTFGQGTKVEI |
| | KRTV |
| | Antibody C14 (SEQ ID NOs: 209-224) |
| HCVR | EVQLVESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCARDTIP |
| | GYMDVWGKGTTVTVSS |
| LCVR | DIMLTQSPSSLSGSVGDSVTFTCRASQSISNYLSWYQQKSGKAPQLLIYAASS |
| | LQSGVPSRFSGSGSGTDFTLTISSLQPEDFATYYCQQSYSSPYTFGQGTKLEIK |
| | RTV |
| | Antibody C15 (SEQ ID NOs: 225-240) |
| HCVR | EVQLVESGGGLVQPGGSLRLSCAASGFTFSRYRMAWVRQAPGKGLEWVSA |
| | IARSGGRTWYADSVKGRFTISRDNSKNTLYLQMNSLRAEDTAVYYCARGG |
| | GAKWLYNWFDSWGQGTLVTV |
| LCVR | EIVLTQSPGTLSLSAGERATLSCRASQSVSNTYLAWYQQKPGQAPRLLIYGA |
| | SIRAPGIPDRFSGSGSGTDFTLTVNRLEPEDSAVYYCQQYARSRIAFGQGTRL |
| | EIRRTV |
| | Antibody C16 (SEQ ID NOs: 241-256) |
| HCVR | EVQLVESGGGLVQPGGSLRLSCAASGFTFSHYWMGWVRQAPGKGLEWVS |
| | GIGASGGWTGYADSVKGRFTISRDNSKNTLYLQMNSLRAEDTAVYYCARTS |
| | GAYFDYWGQGTLVTVSS |
| LCVR | EIVLTQSPGTLSLSPGQRATLSCRASQSVSSDYLAWYQQKPGQAPRLLMYG |
| | ASSRATGIPDRFSGSGSGTDFTLTISRLEPEDFAVYYCQQYGSTPLTFGGGTT |
| | VEIRRTV |
| | Antibody C17 (SEQ ID NOs: 257-272) |
| HCVR | EVQLVESGGGLVQPGGSLRLSCAASGFTFSNYPMTWVRQAPGKGLEWVSTI |
| | RGSGGDTWYADSVKGRFTISRDNSKNTLYLQMNSLRAEDTAVYYCAKWV |
| | GRDAWGQGTLVTVSS |
| LCVR | DIVLTQSPLSLPVTLGQPASISCRSSQSLVYSDGNTYLNWFRQRPGQSPRRLI |
| | YKVSNRDSGVPDRFSGSGSGTDFTLRISRVEAEDVGVYYCMQGTHWPPTFG |
| | QGTKLEIKRTV |
| | Antibody C18 (SEQ ID NOs: 273-288) |
| HCVR | EVQLVESGGGLVQPGGSLRLSCAASGFTFSSYPMNWVRQAPGKGLEWVSRI |
| | SRSGGRTSYADSVKGRFTISRDNSKNTLYLQMNSLRAEDTAVYYCARDATR |
| | RHYNGMDVWGKGTTVTVSS |
| LCVR | DIVLTQSPATLSLSPGERASLSCRASRSVGKYLAWYQQKPGQAPRLLFYYAS |
| | LRAGDIPSRFTASGSGTDFTLTISRLEPEDFAVYYCQQYGSSPRTFGQGTKLE |
| | MKRTV |
| | Antibody C19 (SEQ ID NOs: 289-304) |
| HCVR | EVQLVESGGGLVQPGGSLRLSCAASGFTFSRYRMHWVRQAPGKGLEWVSSI |
| | ASSGGRTRYADSVKGRFTISRDNSKNTLYLQMNSLRAEDTAVYYCARGGLP |
| | YRGHYGMDVWGQGTLVTVSS |
| LCVR | EIMLTQSPSSLSASVGDRVTITCRASQSISSYLNWYQQKPGKAPKLLISVASS |
| | LQSGVPSRFSGSRSGTDFTLTISSLQPEDFATYYCQQARSIPWTFGQGTNVEI |
| | KRTV |
| | Antibody C20 (SEQ ID NOs: 305-320) |
| HCVR | EVQLVESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCARGGLP |
| | YRGHYGMDVWGQGTLVTVSS |
| LCVR | DIVLTQSPLSLPVTPGEPASISCRSSQSLLHSNGYNYVDWYLQKPGQSPQLLI |
| | YLGSNRASGVPDRFSGSGSGTDFTLKISRVEAEDVGVYYCMQALKIPRTFGQ |
| | GTKVEIKRTV |
| | Antibody C21 (SEQ ID NOs: 321-336) |
| HCVR | EVQLVESGGGLVQPGGSLRLSCAASGFTFSPYYMVWVRQAPGKGLEWVSSI |
| | NRSGGRTAYADSVKGRFTISRDNSKNTLYLQMNSLRAEDTAVYYCARAIAA |
| | GRYGMDVWGKGTTVTVSS |
| LCVR | DIEMTQSPATLSLSPGERATLSCRASQSVSSYLAWYQQKPGQPPRLLIYDAS |
| | NRATGIPARFSGSGSGTDFTLTISSLEPEDFAVYYCQQRTNWPPLTFGGGTKV |
| | EIKRTV |
| | Antibody C22 (SEQ ID NOs: 337-352) |
| HCVR | EVQLVESGGGLVQPGGSLRLSCAASGFTFSRYTMRWVRQAPGKGLEWVSGI |
| | SRSGGRTVYADSVKGRFTISRDNSKNTLYLQMNSLRAEDTAVYYCARDPFG |
| | VVNHEYYMDVWGKGTTVTVSS |
| LCVR | EIMLTQSPPSLSASVGDRVTITCRASQSIHTYLNWYQQKPGKAPKLLIYGASN |
| | LQNGVPSRFSGTGSGTDFALTISSLQPEDFATYSCQQTYRTPTTFGPGTKVDI |
| | KRTV |
| | Antibody C23 (SEQ ID NOs: 353-368) |
| HCVR | EVQLVESGGGLVQPGGSLRLSCAASGFTFSSYRMSWVRQAPGKGLEWVSGI |
| | GRSGGRTRYADSVKGRFTISRDNSKNTLYLQMNSLRAEDTAVYYCARAIAA |
| | GRYGMDVWGKGTTVTVSS |
| LCVR | DIMLTQSPGTLSLSPGERATLSCRASQSIRNNYLAWYQQRPGQAPRLLIYGA |
| | SYRATGIPDRFSGSGSGTDFTLTISSLEPEDFAVYYCQQRSNWPPTFGGGTKV |
| | EIKRTV |
| | Antibody C24 (SEQ ID NOs: 369-384) |
| HCVR | EVQLVESGGGLVQPGGSLRLSCAASGFTFSRYPMVWVRQAPGKGLEWVSRI |
| | SRSGGRTQYADSVKGRFTISRDNSKNTLYLQMNSLRAEDTAVYYCARDAT |
| | GRHYNGMDVWGQGTLVTVSS |
| LCVR | EIAMTQSPSSLSASVGDRVTITCRASQSISSYLNWYQQKPGKAPKLLIYGASS |
| | LQSGVPSRFSGSGSGTDFTLTISSLQPEDFATYYCQQANSFPLTFGGGTKVEI |
| | KRTV |
| | Antibody C25 (SEQ ID NOs: 385-400) |
| HCVR | EVQLVESGGGLVQPGGSLRLSCAASGFTFSSYRMSWVRQAPGKGLEWVSGI |
| | GRSGGRTRYADSVKGRFTISRDNSKNTLYLQMNSLRAEDTAVYYCARAIAA |
| | GRYGMDVWGKGTTVTVSS |
| LCVR | EIELTQSPGTLSLSPGERATLSCRASQSIRNNYLAWYQQRPGQAPRLLIYGAS |
| | YRATGIPDRFSGSGSGTDFTLTISSLEPEDFAVYYCQQRSNWPPTFGGGTKVE |
| | IKRTV |
| | Antibody C26 (SEQ ID NOs: 401-416) |
| HCVR | EVQLVESGGGLVQPGGSLRLSCAASGFTFSRYRMAWVRQAPGKGLEWVSG |
| | ISYSGGETLYADSVKGRFTISRDNSKNTLYLQMNSLRAEDTAVYYCARDVR |
| | WLQGLDNWGQGTLVTVSS |
| LCVR | EIKLTQSPLSLPVTPGEPASISCRSSQSLLHTNGNYLDWYLQKPGQSPQLLI |
| | YLGSNRASGVPDRFSGSGSGTDFTLKISRVEAEDVGVYYCMQTLQTPLTFG |
| | GGTKVEIKRTV |
| | Antibody C27 (SEQ ID NOs: 417-432) |
| HCVR | EVQLVESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCAREGRP |
| | GYMDVWGQGTLVTVSS |
| LCVR | EIVLTQSPSLLSASVGDRVTITCRTSLSIATYLHWYQQKPGRAPKLLIYHASS |
| | LQTGVPSRFSGSGSGTDFTLTISSLLPEDFATYFCQQSYSSPYTFGRGTKLEIK |
| | RTV |
| | Antibody C28 (SEQ ID NOs: 433-448) |
| HCVR | EVQLVESGGGLVQPGGSLRLSCAASGFTFSVYGMIWVRQAPGKGLEWVSGI |
| | PPSGGVTLYADSVKGRFTISRDNSKNTLYLQMNSLRAEDTAVYYCARGNYG |
| | MDVWGKGTTVTVSS |
| LCVR | EIALTQSPATLSLSPGERATLSCRASQSVSSYLAWYQQKPGQAPRLLIYDASN |
| | RATGIPARFSGSGSGTDFTLTISSLEPEDFAVYYCQQRSNWPPTFGGGTKVEI |
| | KRTV |
| | Antibody C29 |
| HCVR | EVQLVESGGGLVQPGGSLRLSCAASGFTFSNYPMTWVRQAPGKGLEWVSTI |
| | RGSGGDTWYADSVKGRFTISRDNSKNTLYLQMNSLRAEDTAVYYCAKWV |
| | GRDAWGQGTLVTVSS |
| LCVR | DIQLTQSPLSLPVTLGQPASISCRSSQSLVYSDGNTYLNWFQQRPGQSPRRLI |
| | YKVSNRDSGVPDRFSGSVSGPDFTLKISRVEAEDVGVYYCMQGTHWPYTFG |
| | QGTKLEIKRTV |

In the tables below, only the HCVR and LCVR sequences are given SEQ ID NOs.

| | |
|---|---|
| | Antibody C30 (B34) (SEQ ID NOs: 563-564) |
| HCVR | EVQLLESGGGLVQPGGSLRLSCAASGFTFSSYAMSWVRQAPGKGLEWVSGI |
| | TGSGGSTYYADSVKGRFTISRDNSKNTLYLQMNSLRAEDTAVYYCARGRQ |
| | VDKWGQGTLVTVSS |
| LCVR | DIMLTQSPSSLSASVGDRVTITCRASQSIHNYLNWIQQTPGKAPKLLVFAAST |
| | LQRGVPPRYSGRGSGTEFTLTISSLQPEDFATYYCQQSYTTPLTFGGGTKVEI |
| | RRTV |
| | Antibody C31 (B46) (SEQ ID NOs: 565-566) |
| HCVR | QVQLVESGGGVVQPGRSLRLSCAASGFTESTYGMHWVRQAPGKGLEWVA |
| | VIWDDGSYKYYGDSVKGRFTISRDNSKNTLYLQMNSLRAEDTAVYYCAGG |
| | YSYSYMAIWGKGTTVTVSS |
| LCVR | DIVLTQSPSSLSASVGDRVTITCRASQGISTFLNWYQQKPGKAPKLLIYAASS |
| | LQSGVPSRFSGSGSGTDFTLTIGSLQPEDFASYFCQQTYSTQWTFGQGTKVEI |
| | KRTV |
| | Antibody C32 (B104) (SEQ ID NOs: 567-568) |
| HCVR | QVQLVQSGAEVKRPGASVKVSCKASGYTFTNDIIHWVRQAPGQRLEWMG |
| | WINAGYGNTQYSQNFQDRVSITRDTSASTAYMELISLRSEDTAVYYCARGY |
| | YRSPTWFDWWGQGTLVTVSS |
| LCVR | ETTLTQSPSSLSASVGDRVTITCRASQSISSWLAWYQQKPGKAPKLLIYKASS |
| | LESGVPSRFSGSGSGTEFTLTISSLQPDDFATYYCQQYGDYPYTFGQGTKLEI |
| | KRTV |
| | Antibody C33 (1C2) (SEQ ID NOs: 569-570) |
| HCVR | QVQLVESGGGVVQPGRSLRLSCAASGFTESTYGMHWVRQAPGKGLEWVA |
| | VIWDDGSYKYYGDSVKGRFTISRDNSKNTLYLQMNSLRAEDTAVYYCARD |
| | GSGWGYAFDIWGQGTMVTVSS |
| LCVR | DIQLTQSPSSLSASVGDRVTITCQASQDISEYLNWYQHKPGKAPNPLVFDAA |
| | NLETGVPPRFSATGSGTDFSLTITSLQPEDFATYYCQQYDSFPYTFGQGTRLD |
| | IKRTV |
| | Antibody C34 (1D7) (SEQ ID NOs: 571-572) |
| HCVR | EVQLLESGGGLVQPGGSLRLSCAVSGFTFNSFAMSWVRQAPGKGLEWVSAI |
| | SGSGGGTYYADSVKGRFTISRDNSKNTLYLQMNSLRAEDTAIYYCAREVDG |
| | YGAFDIWGQGTMVTVSS |
| LCVR | DIQMTQSPSSLSASVGNRVTITCRASQTIRNYLNWYQQKPGEAPKVLIYGAS |
| | TLQSGVPSRFSASGSGTDFTLTISSLQPDDFATYHCQQTESDPYTFGQGTKVE |
| | IKRTV |
| | Antibody C35 (1B1) (SEQ ID NOs: 573-574) |
| HCVR | EVQLLESGGGLVQPGGSLRLSCAVSGFTFNSFAMSWVRQAPGKGLEWVSAI |
| | SGSGGGTYYADSVKGRFTISRDNSKNTLYLQMNSLRAEDTAVYYCAREGW |
| | EVRTPGYFDYWGQGTLVTVSS |
| LCVR | DVVMTQSPSSLSASVGDRVTITCRASHRIFSYLTWYHQKPGKAPNLLISGAS |
| | SLGAGVPSRFSGSGSGTDFTLTISSLQPEDFGTYYCQQSFSDPYTFGQGTKL |
| | EIKRTV |
| | Antibody C36 (1B4) (SEQ ID NOs: 575-576) |
| HCVR | EVQLLESGGGLVQPGGSLRLSCAASGFTFSSYAMSWVRQAPGKGLEWVSGI |
| | TGSGGSTYYADSVKGRFTISRDNSKNTLYLQMNSLRAEDTAIYYCARGGAL |
| | DIWGQGTMVTVSS |
| LCVR | DIQMTQSPSSLSASVGDRVTITCRASQVIHNYLAWYKQKPGQAPKLLIYSAS |
| | SLQSGVPSRFSGSGSGTDFTLTISSLQPEDFATYYCQQSYSTPLTFGGGTKV |
| | EIKRTV |
| | Antibody C37 (3F12) (SEQ ID NOs: 577-578) |
| HCVR | EVQLVQSGGGLVKPGGSLRLSCAASGFTFSSYSMNWVRQAPGKGLEWVSSI |

| | |
|---|---|
| | SSSSSYIYYADSVKGRFTISRDNAKNSLYLQMNSLRAEDTAVYYCAGRQYD |
| | FWSGWDWEDPWGQGTLVTVSS |
| LCVR | DIQLTQSPSSLSASVGDRVTITCRASQSISTYLNWYQQKPGKAPKLLIHDASIL |
| | QSGVPSRFSGSGSGTDFTLTISSLQAEDFATYYCQQSYSTLPYTFGQGTKVEI |
| | KRTV |
| | Antibody C38 (2B4) (SEQ ID NOs: 579-580) |
| HCVR | EVQLVESGGGLEQPGGSLRLSCAGSGFTFRDYAMTWVRQAPGKGLEWVSSI |
| | SGSGGNTYYADSVKGRFTISRDNSKNTLYLQMNSLRAEDTAIYYCARGEYS |
| | SGWPSWFDPWGQGTLVTVSS |
| LCVR | EIVLTQSPSSLSASVGDRVTISCRASRTVGTFLNWYQQRPGKPPQALILGVSIL |
| | EGGVSSRFSGAGSGTEFSLTISSLQPEDIGTYYCQQSHDVPRTFGQGTKVDLR |
| | RTV |
| | Antibody C39 (8G4) (SEQ ID NOs: 581-582) |
| HCVR | EVQLLESGGGLVQPGGSLRLSCAASGFTESSYAMSWVRQAPGKGLEWVSGI |
| | TGSGGSTYYADSVKGRFTISRDNSKNTLYLQMNSLRAEDTAVYYCARSRNL |
| | DSWGQGTLVTVSS |
| LCVR | DIQLTQSPSSLSASVGDRVTITCRASQSIHNYLNWIQQTPGKAPKLLVFAAST |
| | LQRGVPPRYSGRGSGTEFTLTISSLQPEDFATYYCQQSYTTPLTFGGGTKVEI |
| | RRTV |

In all the above sequences, HCVR (heavy chain variable region) sequence can be assembled based on the disclosed sequences of HFR1/CDR-H1/TIVR2/CDR-H2/TIVR3/CDR-H3/TIVR4 (N to C terminus), plus the most N-terminal signal peptide sequence of MIISSALLCCLVLLTGVRA (SEQ ID NO: 465).

Likewise, LCVR (light chain variable region) sequence can be assembled based on the disclosed sequences of LFR1/CDR-L1/LFR2/CDR-L2/LFR3/CDR-L3/LFR4 (N to C terminus), plus the most N-terminal signal sequence of MHSSALLCCLVLLTGVRA (SEQ ID NO: 465).

One human light chain constant region sequence is shown below:

(SEQ ID NO: 466)
AAPSVFIFPPSDEQLKSGTASVVCLLNNFYPREAKVQWKVDNALQSGN
SQESVTEQDSKDSTYSLSSTLTLSKADYEKHKVYACEVTHQGLSSPVT
KSFNRGEC

The human IgG1 heavy chain constant region sequences are shown as follows:

(SEQ ID NO: 467)
ASTKGPSVFPLAPSSKSTSGGTAALGCLVKD
YFPEPVTVSWNSGALTSGVHTFPAVLQSSGL
YSLSSVVTVPSSSLGTQTYICNVNHKPSNTK
VDKKVEPKSCDKTHTCPPCPAPELLGGPSVF
LFPPKPKDTLMISRTPEVTCVVVDVSHEDPE
VKFNWYVDGVEVHNAKTKPREEQYNSTYRVV
SVLTVLHQDWLNGKEYKCKVSNKALPAPIEK
TISKAKGQPREPQVYTLPPSRDELTKNQVSL
TCLVKGFYPSDIAVEWESNGQPENNYKTTPP
VLDSDGSFFLYSKLTVDKSRWQQGNVFSCSV
MHEALHNHYTQKSLSLSPGK

Although for the in vivo assays described in this application, only the human IgG1 anti-IGSF8 antibodies were used, other anti-IGSF8 antibodies with other Ig constant regions (such as IgG2, IgG3, IgG4, IgA, IgE, IgM, IgD constant regions) are also contemplated and within the scope of the invention.

### Example 8 Anti-IGSF8 Antibodies Exhibit Strong ADCC Effects

This experiment demonstrates that anti-IGSF8 antibodies of the invention exhibit strong ADCC effects using NK cells as effector cells and A431 cancer cells as target cells.

Here, ADCC (antibody-dependent cell-mediated cytotoxicity) stands for an immune response in which antibodies, by coating target cells, make them vulnerable to attack by immune cells. Specifically, IGSF8 expressed on A431 cancer cell surface was recognized and bound by an increasing concentration of anti-IGSF8 antibodies. The Fc regions of the anti-IGSF8 antibodies were in turn recognized by CD16 Fc receptors on NK cells. Cross-linking of the CD16 Fc receptors triggers a degranulation into a lytic synapse. As a result, the targeted tumor cells were killed via apoptosis.

A431 cells were seeded in 96-well plates with RPMI medium, and incubated for about 1 hour with varying concentrations of the anti-IGSF8 isotypes. Activated primary NK cells from donors were then added to the A431 cells- and antibody-containing wells at 4,000 cells/well (a target:effector ratio of 1:2.5), and incubated for 4 more hours at 37° C. Cell death was determined by lactate dehydrogenase (LDH) release assays.

A dose-response curve was established for each of the 12 tested antibodies C1-C12, and their EC₅₀ values were determined.

All 12 tested anti-IGSF8 antibodies (C1-C12) showed about 3-12 mM range ADCC EC₅₀ values against the A431 cancer cells.

### Example 9 Anti-IGSF8 Antibodies Stimulate CXCL10 Expression

FIG. 4 above shows that inactivating IGSF8 in Colo205 cancer cells using CRISPR/Cas9-mediated gene editing caused a near 7-10 fold increased expression/secretion of CXCL10 by Colo205 cells. This experiment shows that incubating the Colo205 cancer cells with the anti-IGSF8 antibodies of the invention (10 μg/mL) can similarly lead to CXCL10 expression/secretion, based on ELISA.

Specifically, Colo205 cancer cells were seeded in 96 well plates (4,000 cells per well) and cultured with RPMI medium for 12 hours, before one of the test antibodies was added at g/mL for 24 hours at 37° C. in a humidified atmosphere of 5% CO₂. The supernatant of the media was then collected for standard ELISA assay to determine the titer/amount of CXCL10 in the medium by using a commercial CXCL10 ELISA kit. Antibodies C1-C4, C8, and C10 all induced relatively high levels of CXCL10 expression by Colo205 cells.

### Example 10 Anti-IGSF8 Antibodies Showed In Vivo Efficacy

In FIGS. 3A-3B, it was shown that knocking out IGSF8 using CRISPR/Cas9-mediated gene editing led to retarded B16-F10 melanoma growth in vivo in a mouse xenograph model, without affecting in vitro tumor cell growth rate per se.

In this experiment, the effect of representative anti-IGSF8 monoclonal antibodies of the invention on tumor growth in B16 syngeneic mouse model was tested. In particular, one million B16-F10 melanoma cells were injected subcutaneously into wild type (WT) C57BL/6 mice. Mice were then treated with one of four anti-IGSF8 antibodies (C1-C4) at a dose of 2 mg/kg, or a control human IgG1, from day 6, every 3 days, for four doses in total by tail vein injection. Data are presented as mean±s.e.m. (n=8 mice per group).

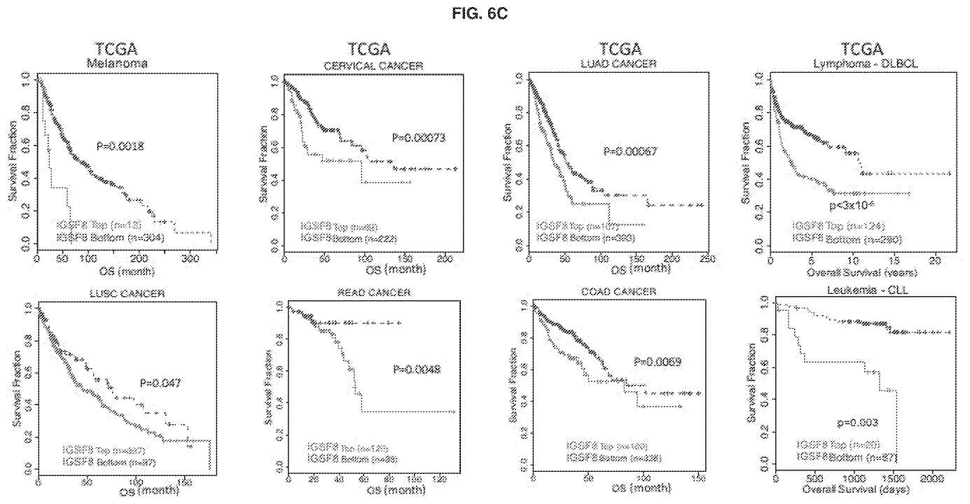

It is apparent that, in wild-type host mice, the subject anti-IGSF8 monoclonal antibodies similarly retarded B16-F10 melanoma tumor growth (volume increase), such that the difference compared to the IgG1 control became statistically significant (p<0.005) after about 18 days for at least C3 and C4. See FIG. 10.

Similar experiments were repeated in nude mice (Foxn1^{nu}), which lack thymus and cannot produce mature T lymphocytes, but have B cells and robust NK cell responses. The effects of the subject anti-IGSF8 antibodies appeared to be similar. At Day 14, the effect of the C2 antibody was statistically significant (p<0.05), so was the effect of C4 (p<0.005).

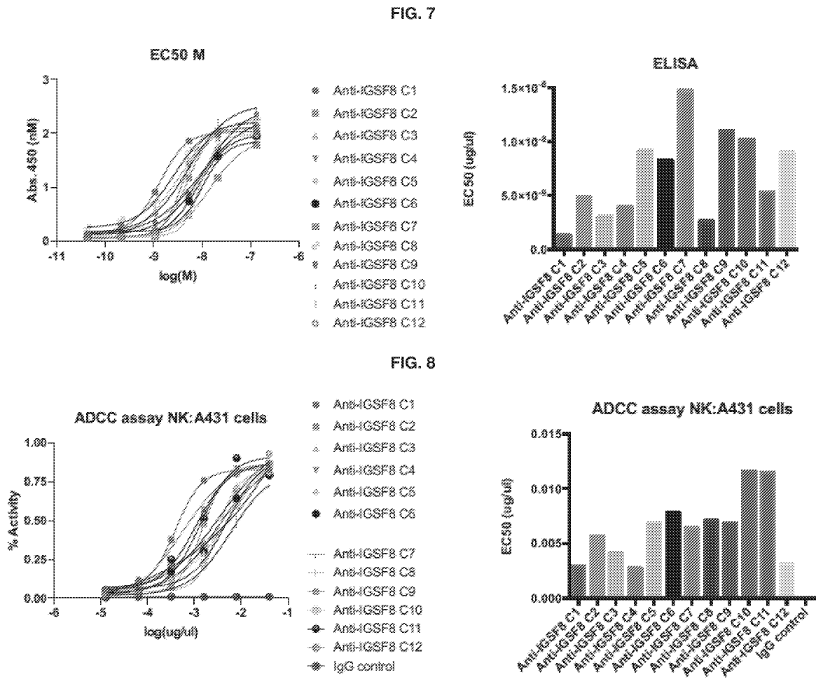

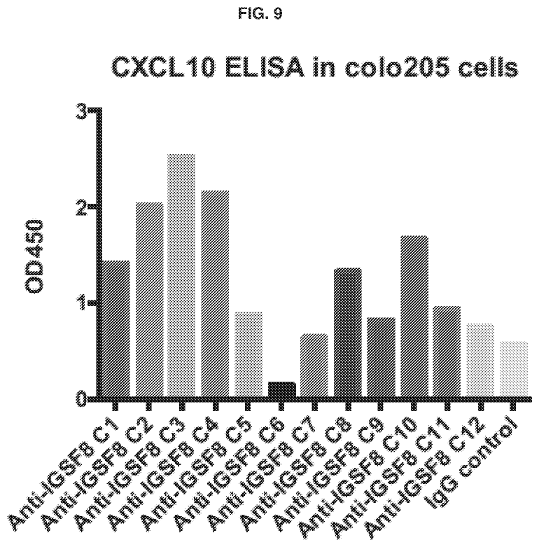

Notably, there did not appear to be any significant weight differences among the different groups of experimental mice (FIG. 11), which result was consistent with the fact that knocking out IGSF8 using CRISPR/Cas9 did not have appreciable effect on tumor cell growth rate per se.

### Example 11 Synergistic Anti-Tumor Effect by Anti-IGSF8 Antibody and Anti-PD-1 Antibody

This experiment demonstrates that the anti-IGSF8 monoclonal antibodies of the invention and anti-PD-1 antibody have synergistic effect in inhibiting B16-F10 melanoma tumor growth in vivo in a syngeneic mouse model.

In particular, one million B16-F10 melanoma cells were injected subcutaneously into wild type (WT) C57BL/6 mice. Mice were then treated, by tail vein injection, with one of four antibodies or antibody combinations: IgG control at a dose of 2 mg/kg, anti-PD-1 antibody at a dose of 2 mg/kg, anti-IGSF8 antibody C3 at a dose of 2 mg/kg, or a combination of anti-PD-1 antibody at half the dose (1 mg/kg) and anti-IGSF8 antibody at half the dose (1 mg/kg). The first doses were administered on Day 6, and subsequent doses were administered every 3 days, for four doses in total. Data are presented as mean±s.e.m. (n=8 mice per group).

It is apparent that the subject anti-IGSF8 antibody and anti-PD-1 antibody exhibited synergistic effect in inhibiting melanoma growth in vivo, in that the combination therapy, administered at a 50% dose (1 mg/kg) for each component of the combination, was statistically significantly better than (1) the anti-IGSF8 antibody C3 alone at twice the dose (2 mg/kg) (p<0.01), (2) the commercial anti-PD-1 antibody (Clone 29F.1A12, BioXcell) alone at twice the dose (2 mg/kg) (p<0.005), and (3) IgG control (p<0.001).

This surprising finding strongly suggests that simultaneously inhibiting the IGSF8 pathway and the PD-1/PD-L1 immune checkpoint can synergistically inhibit tumor growth in vivo.

### Example 12 In Vivo Genetic Screens to Uncover Immune Suppression Genes

This example illustrates the use of a pooled genetic screening approach developed to identify genes that increase or decrease the “fitness” of tumor cells growing in vivo in animals treated with immunotherapy (FIG. 1A).

Specifically, a library of lentiviral vectors (which encoded about 6,000 sgRNAs) were created to target about 1,000 genes from relevant functional classes that were expressed at detectable levels in a mouse melanoma cell line B16-F10. The screening was designed to identify genes that, when down-regulated by CRISPR/Cas-mediated knock-down, change fitness score of tumor cells.

After transduction and in vitro passage of the B16-F10 tumor cells to allow gene editing to take place, the B16-F10 cells were transplanted into wild-type C57BL/6 mice, which were then treated with either human IgG1-Fc fragments or anti-PD1, anti-PD-L1, or anti-CTLA4 monoclonal antibodies to generate an adaptive immune response sufficient to apply immune-selective pressure on the tumor cells. In parallel, the library-transduced B16-F10 cells were maintained in vitro.

After 14 days, the tumors and cells were collected. Library representation in tumors isolated from the immunotherapy-treated mice was then compared to that of the cells maintained in vitro. Immunosuppressive genes that, when deleted by CRISPR, specifically decreased tumor cells growth in vivo (in mice), but did not affect cell growth in vitro, were identified.

IGSF8, as well as three well-known immunomodulators (CD47, PDL1 and IFNGR1), were markedly depleted in tumors treated with either IgG or PD1, PDL1, CTLA4 blockade (data not shown). Therefore, this example demonstrates that in vivo genetic screening recovered genes known to confer immune evasion properties on cancer cells.

### Example 13 IGSF8 Human Fc Fusion Protein Suppresses Proliferation and Cytolytic Activity of Activated Primary NK or T Cells from Healthy Donors

Example 2 above demonstrates the negative impact of IGSF8 on NK or T cell activity, in that increasing concentrations of recombinant human IGSF8 extracellular domain tagged by a human Fc region (IGSF8-hFc), when incubated with primary human NK cells or T cells isolated from healthy donors, reduced the viability of these primary NK or T cells over IGSF8-hFc concentrations (dose response curve). See FIGS. 2A and 2B.

A similar experiment was repeated here. Briefly, the primary NK or T cells were isolated from healthy donors' peripheral blood mononuclear cells (PBMCs) using commercial negative/positive isolation kits (StemCell Technologies, Inc.). NK cells were cultured and activated in RPMI medium supplemented with 10% Fetal Bovine Serum (FBS), penicillin/streptomycin, L-glutamine, non-essential amino acids, sodium pyruvate, HEPES, 2-Mercaptoethanol, feeder cells (ARK company, Hangzhou) and recombinant human IL-2 (1,000 IU/mL), and were incubated at 37° C. with 5% CO₂. The T cells were cultured in TexMACS Medium with human IL-2 (1,000 IU/mL) at 37° C. with 5% CO₂, and were activated by anti-CD3 and anti-CD28 mAb once a week.

The primary NK cells were then seeded in 96-well plates (about 3,000 cells per well), and cultured 18 to 24 hours before the IGSF8-hFc fusion protein (5 μg/mL) or human Fc protein (5 μg/mL, as negative control) was added. Cell viability was determined by Cell Counting Kit 8 (CCK8) method with three biological replicates after 72 hours. Two biological replicates of the recombinant IGSF8 human Fc fusion proteins—IGSF8-hFc0601 and IGSF8-hFc0604—were used in the experiments. The results again showed that the IGSF8-hFc fusion protein statistically significantly (p<0.005) inhibited cell proliferation of primary NK cells in a dose dependent manner. See FIG. 2C.

To further understand the mechanism by which the IGSF8-hFc fusion protein inhibits NK cell activity, RNA-seq was performed for the NK cells treated with either hFc (5 μg/mL, negative control) or IGSF8-hFc protein (5 μg/mL) for 24 hours. By comparing differential gene expression between these two groups of cells, the cell cycle gene pathway was identified as the most enriched Gene Ontology (GO) term that was down-regulated in NK cells treated with IGSF8-hFc protein versus those treated with hFc protein (FIG. 2D). However, no cell death gene pathway was significantly down-regulated in the NK cells treated with IGSF8-hFc. The relative mRNA expression of the genes in NK cells treated with IGSF8-hFc fusion protein or hFc control protein was shown in FIG. 2E. These results were consistent with cell proliferation assay results in PBMC cells (see FIG. 2F, in which IGSF8-hFc fusion protein inhibited NK cell proliferation from Division 2 and beyond).

Meanwhile, for T cell proliferation measurement, CD4⁺ T cells were labeled with CFSE (Thermo) according to manufacturer's instruction, before IGSF8-hFc fusion protein (5 μg/mL) or human Fc protein (5 μg/mL, as a negative control) was added, and T cells were cultured for another 24 hours. The data were analyzed by flow cytometry. Interestingly, proliferation of CD4⁺ T cells was also remarkably suppressed by IGSF8-hFc treatment (FIG. 2G).

Since activity of several genes representing the cytolytic activity of activated NK cells (Gzmb, Prfl, etc.) were found to have significantly decreased in the NK cells treated with IGSF8-hFc by RNA-seq (FIG. 2E), NK cell cytolytic activity was analyzed to see if IGSF8-hFc treatment can affect this activity.

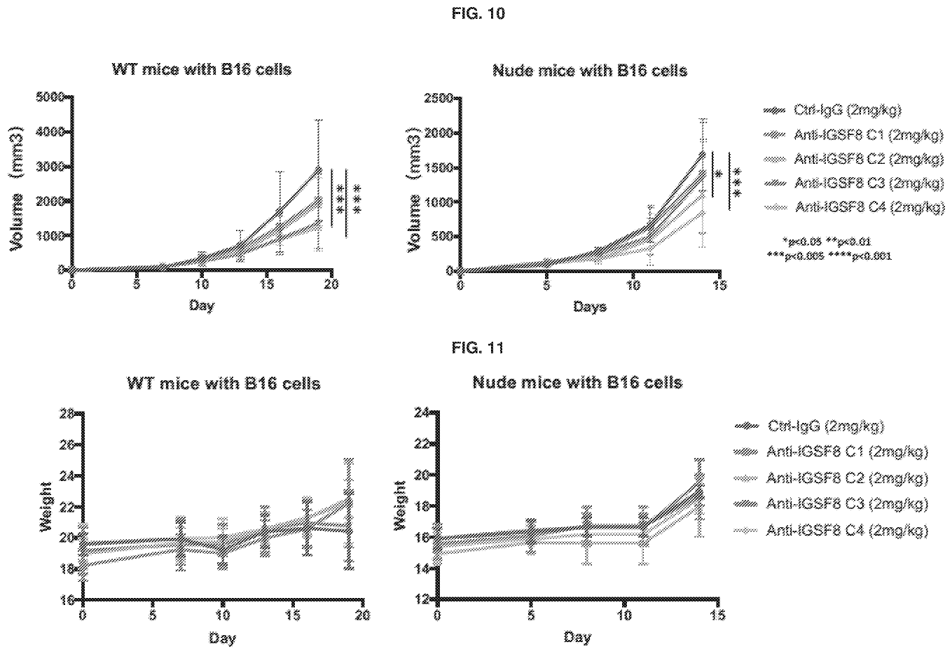

Using a cell co-culture model of NK and K562 cells, the cytolytic activity of the NK cells was found to be dramatically impaired by IGSF8-hFc treatment (FIG. 13A), due to the depleted perforin expression in the NK cells upon IGSF8-hFc treatment (FIG. 13B).

In addition, using CD69 as an activation marker, CD4⁺ T cell activation was also strongly inhibited by IGSF8-hFc treatment (FIG. 2H).

Together, these data strongly suggested that the IGSF8-hFc fusion protein functioned as a suppressor of NK- or T cell-mediated immune response. Thus, the fusion protein may be useful as a therapeutic agent to down-regulate undesired T- and/or NK-cell mediated immune reaction, such as in treating autoimmune disease or acute rejection of organ transplantation.

### Example 14 Increased Binding of Recombinant IGSF8 to Activated NK and T Cells

This example demonstrates that NK and T cells both express receptors for IGSF8.

In this experiment, vectors were constructed to express fusion proteins consisting of the extracellular domain of human IGSF8 fused to an 8×His tag (for purification) and an AVI-tag (for biotinylation by the Biotin Protein Ligase (GeneCopoeia)). The purified recombinant proteins with biotin were then used to detect the presence of a putative IGSF8 receptor on NK/T cells via flow cytometry analysis.

Although the tagged recombinant IGSF8 weakly binds freshly isolated human NK/T cells, about 40% of the CD56+NK cells activated by feeder cell co-culturing, and about 20% of CD4⁺ T cells stimulated by anti-CD3 mAb, were stained positive (FACS data not shown).

These data showed that activated human NK and T cells both expressed putative receptor(s) for IGSF8.

### Example 15 Expression of IGSF8 on Target Cell Surface Affects Cytolytic Activity of Primary NK Cells

This example demonstrates that expression of IGSF8 on the surface of a target cell could modulate NK cell killing activity.

For this experiment, an NK cell-K562 co-culture model was used, in which the MHC-I deficient K562 cell line—a known NK-sensitive cell line—was chosen as the target cell line for NK cell-mediated killing. Specifically, primary NK cells were seeded in 96-well plates (10,000 cells per well) and co-cultured with K562 cells at E/T ratio=5:1 for 2 hours. The dead cells were stained by 7-AAD (Biolegend) before the cells were analyzed by flow cytometry. NK cell cytotoxicity was calculated by NK cytotoxicity (%)=(7-AAD+%(dead) K562 cells cocultured with NK cells)−(spontaneously 7-AAD+%(dead) K562 cells without coculture with NK cells).

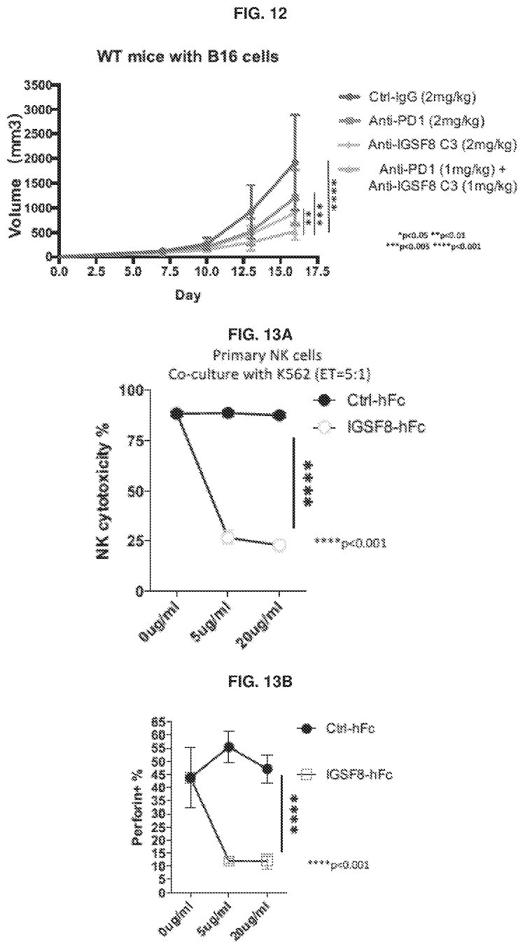

First, forced expression of IGSF8 protein on K562 cells by lentiviral delivery of an IGSF8-expression construct mostly protected the IGSF8-expressing K562 cells from primary NK cell killing. See FIG. 14. NK cells from two unrelated donors were used in this experiment.

Second, loss of IGSF8 expression by lentiviral-mediated CRISPR/Cas9 knock down of any endogenous IGSF8 expression in K562 cells remarkably increased the susceptibility of the K562 cells to primary NK cells obtained from the two unrelated donors (FIG. 14). This result was quite robust, and was consistent in the same assay when using primary NK cells from ten different donors (data not shown).

These data suggested that IGSF8 is a novel and crucial checkpoint gene to regulate NK cell-mediated immune surveillance, and overexpression of IGSF8 on cancer cells with deficient MHC class I can evade NK cell-mediated immune surveillance of cancer.

### Example 16 Domain 1 is the Main Functional Domain of IGSF8 to Suppress NK Cell Immunity

This example demonstrates that Domain 1 (but not other extracellular parts) of IGSF8 is crucial for suppressing NK cell-mediated killing.

IGSF8 has 4 Ig-like topological extracellular domains. See domain organization below.

| | Amino Acid | |
|---|---|---|
| IGSF8 Protein | Position | Description |
| Signal Peptide | 1-27 | — |
| Topological Domain 1 (D1) | 28-149 | Ig V-set domain |
| Topological Domain 2 (D2) | 162-286 | Ig-like C2 domain |
| Topological Domain 3 (D3) | 303-424 | Ig-like C2 domain |
| Topological Domain 4 (D4) | 431-560 | Ig-like C2 domain |
| Transmembrane Domain (TM) | 580-600 | — |
| Cytoplasmic Tail | 601-613 | — |

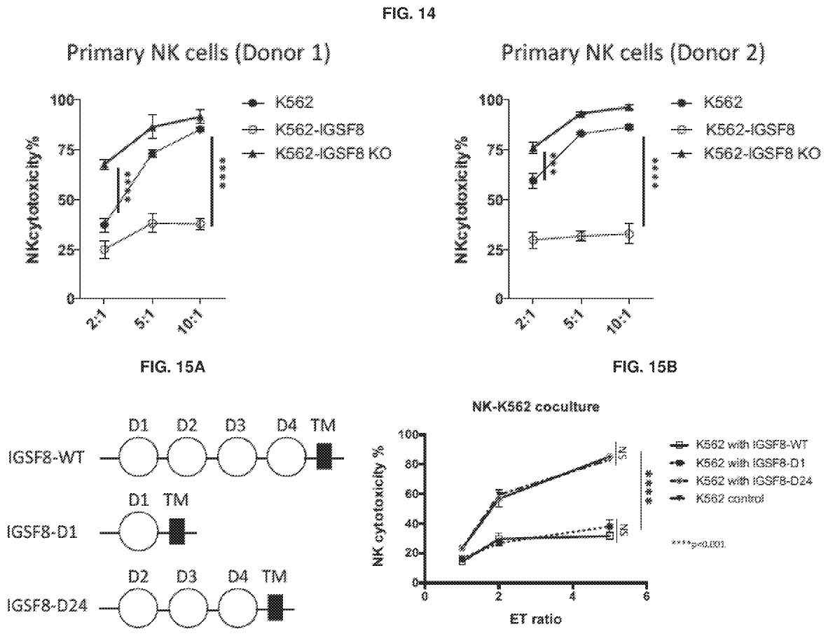

Two truncated version of IGSF8 were constructed, one (“D1”) expression the first (outer-most) extracellular domain D1, the other (“D24”) expressing the remaining three (D2-D4) extracellular domains closer to the transmembrane domain. See FIG. 15A. Both constructs were force expressed on K562 cells for use in the co-culture experiments as in Examples 14 and 15.

The first domain of IGSF8 (D1) containing a Ig V-set domain was demonstrated to be the main functional domain for NK cell binding (FIG. 15B), while the truncated IGSF8 protein (D24) without D1 domain completely lost the suppressive functions of IGSF8 for NK cell immunity (FIG. 15B).

This data demonstrates that the D1 domain is the major (if not only) functional domain responsible for NK cell suppression. Targeting the D1 domain by various types of inhibitors (e.g., small molecule, antibody, peptide or nucleic acid) can modulate (inhibit) NK cell-based immunotherapy.

### Example 17 KIR3DL1/2 and KLRC1&D1 Heterodimer are IGSF8 Receptors of NK Cells

This example demonstrates that KIR3DL1/2 and KLRC1&D1 heterodimers are IGSF8 receptors on NK cells, and are potential drug targets for modulating IGSF8-mediated NK cell immunity.

Example 14 showed that recombinant IGSF8 protein can stably bind to activated primary NK cells, suggesting that NK cells express a receptor(s) for IGSF8.

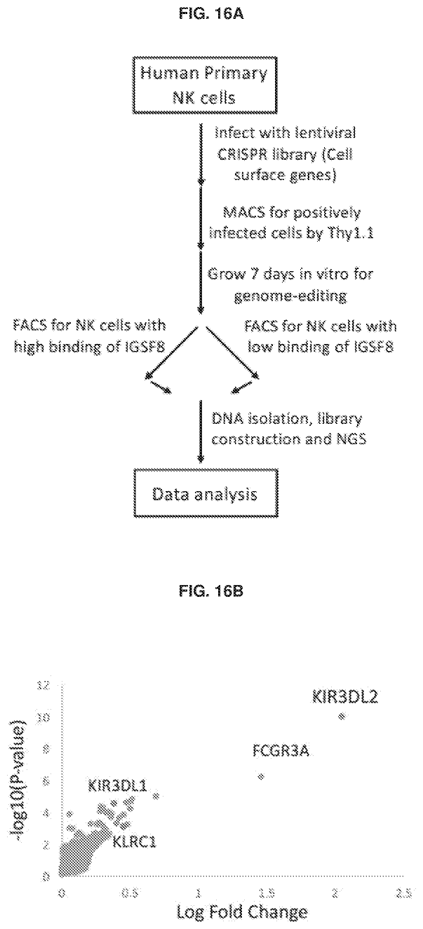

In order to identify the IGSF8 receptor, CRISPR screens were performed for all human cell surface proteins in primary NK cells isolated from health donors. Specifically, Fluorescence-activated cell sorting (FACS) was used to isolate IGSF8 high- or low-binding cells after cell transduction with lentiviral CRISPR libraries, followed by high-throughput gRNA sequencing. Model-based Analysis of Genome-wide CRISPR/Cas9 Knockout (MAGeCK) software was subsequently used to count the gRNA reads, and to perform the gene/gRNA fold change, selection score and statistical analyses, between IGSF8 high- and low-binding cell samples. See FIG. 16A.

Based on the above analysis, KIR3DL2 was identified as the top hit, and KIR3DL1, KLRC1 were also shown as an outlier hits, indicating that the loss of expression of these genes on the NK cells reduced IGSF8 binding (FIG. 16B).

Both KIR3DL2 and KIR3DL1 belong to the Killer-cell immunoglobulin-like receptors (KIRs), which are a family of type I transmembrane glycoproteins expressed on the plasma membrane of natural killer (NK) cells and a minority of T cells. At least 15 genes and 2 pseudogenes encoding KIR family proteins map in a 150-kb region of the leukocyte receptor complex (LRC) on human chromosome 19q13.4. The KIR receptors are thought to regulate the killing function of these cells by interacting with major histocompatibility (MIC) class I molecules expressed on all nucleated cell types. Most KIRs are inhibitory, in that their recognition of MIC molecules lead to suppression of the cytotoxic activity of NK cell expressing the KIRs. These inhibitory KIRs have comparatively longer cytoplasmic tails (hence the “L” in their gene name), with one or two so-called ITIM motifs. Only a limited number of KIRs are activating, in that their recognition of the MHIC molecules activates the cytotoxic activity of NK cell expressing the activating KIRs. These activating KIRs have short cytoplasmic tails (hence the “S” in their gene names), without ITIM motifs. A common feature of the activating KIR (aKIRs) is the presence, in their transmembrane region, of a charged residue (Lys) that allows their association with the signaling adaptor protein KARAP/DAP12 containing immunoreceptor tyrosine-based activating motifs (ITAMs).

KLRC1 (Killer cell Lectin-like Receptor, subfamily C, Member 1) also known as NKG2A or NKG2B as alternative splicing variants, is one of a family of transmembrane proteins preferentially and primarily expressed in NK cells and implicated in regulating NK cell function, characterized by the unusual type II membrane orientation (extracellular C terminus) and the presence of a C-type lectin domain. The NKG2 genes are also differentially regulated in T cells. The limited distribution of these proteins and their sequence similarity with known receptor molecules befit its function as a receptor for NK cells. Like the KIR receptors, NKG2A has 2 ITIMs that interact with SHP1 or SHP2. NKG2A is typically expressed on about half of all NK cells as well as on a subset of CD8⁺ T cells. NKG2A also recognizes HLA-E, a nonclassical MHC molecule of limited sequence variability.

KLRD1 (Killer cell Lectin-like Receptor, subfamily D, Member 1), also known as CD94, is expressed as 3 major transcripts of 0.8, 1.8, and 3.5 kb and a minor transcript of 5.5 kb in NK cell lines. Like KLRC1, KLRD1 is also an unusual Type II membrane protein with an external C terminus. The predicted protein contains a 147-amino acid extracellular domain with several motifs characteristic of C-type lectins, a 26-amino acid transmembrane domain, and essentially no cytosolic tail except for a 7-amino acid cytoplasmic domain. CD94 shares low sequence identity (between 27-32%) with the NKG2 family proteins including NKG2A (KLRC1). The virtual absence of a cytoplasmic domain is consistent with the finding that KLRD1/CD94 must form a heterodimer with KLRC1 to bind IGSF8. KLRD1 has been shown to form disulfide-bonded heterodimers with NKG2A (KLRC1), NKG2C and NKG2E. In addition, HLA-E tetramer has been found to bind to natural killer (NK) cells and a small subset of T cells from peripheral blood, which may be through binding to the CD94/NKG2A (KLRD1/C1 heterodimer), CD94/NKG2B, and CD94/NKG2C NK cell receptors, but HLA-E tetramer does not bind to the immunoglobulin family of NK cell receptors KIR.

As the KIR receptors share strong homology within the KIR family of receptors, further experiments were conducted to show that IGSF8 specifically binds to KIR3DL1/2.

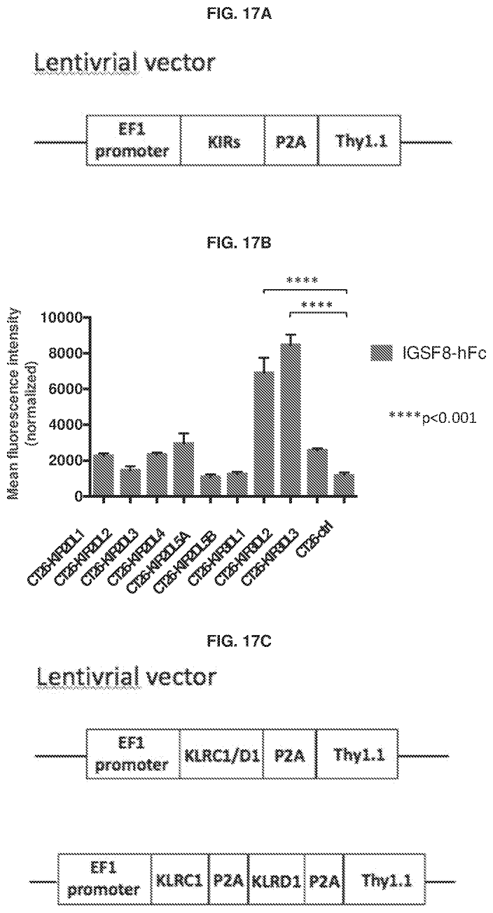

For this experiment, different human KIR receptors (KIR2DL1, KIR2DL2, KIR2DL3, KIR2DL4, KIR2DL5A, KIR2DL5B, KIR3DL1, KIR3DL2, KIR3DL3) were first cloned into a proprietary lentiviral vector containing a thy1.1 tag (FIG. 17A). Then mouse CT26 cells, were transducted by these lentiviral constructs, and IGSF8 binding was tested with biotin-labelled recombinant IGSF8 hFc fusion protein. The mean fluorescence intensity (MFI) of IGSF8 binding to different CT26 cells were normalized by the expression of different KIRs on CT26 cells using an anti-thy1.1 mAb (Biolegend) via flow cytometry.

Selectivity of IGSF8 binding to KIR3DL1-and KIR3DL2-expressing CT26 cells was demonstrated, because no or at best weak binding against other KIR receptors (KIR2DL1, KIR2DL2, KIR2DL3, KIR2DL4, KIR2DL5A, KIR2DL5B, KIR3DL3) was observed under the experimental conditions (FIG. 17B).

Like the other inactivating KIRs, the cytoplasmic tail of KIR3DL1/2 contains an ITIM motif comprised of the sequence “VTYAQL,” indicating that KIR3DL1/2 is an inhibitory receptor for IGSF8 that can serve as a checkpoint receptor target for cancer immunotherapy.

Since KLRC1 was also shown as a hit from previous screens, additional experiments were conducted to confirm that this is also a receptor for IGSF8.

KLRC1 also contains a ITIM motif in its intracellular domain, and was known to be a inhibitory receptor of NK or CD8⁺ T cells as part of a heterodimer with KLRD1 on the cell surface. The data provided herein demonstrates that IGSF8 specifically binds to cells co-transfected by KLRC1 and KLRD1, but not cells transduced with either KLRC1 or KLRD1 lentiviral constructs alone (FIG. 17C & 17D). Specifically, 100,000 CT26 cells overexpressing the antigens (KIRs or KLRs) were washed with PBS buffer containing 2% FBS, and incubated with 100 μL IGSF8 recombinant proteins or antibodies (5 μg/mL) for 30 minutes on ice. Cells were then washed twice with wash buffer, and incubated with 100 μL of 1:100 anti-human Fc-PE antibody for 15 minutes on ice. Cells were washed twice with wash buffer and analyzed on a FACS analyzer (CytoFlex, Beckman). In these experiments, the mean fluorescence intensity (MFI) of IGSF8 binding to different CT26 cells were normalized by the expression of different KLRs on CT26 cells using an anti-thy1.1 mAb via flow cytometry.

Consistent with the data in Example 16, the D1 domain of IGSF8 protein specifically binds to the KIR3DL1/2 and KLRC1&D1 heterodimer receptor on the cell surface, while the IGSF8 protein without D1 domain was unable to bind to these receptors (FIG. 17E).

Together, these data showed that KIR3DL1/2, and the KLRC1&D1 heterodimer receptors, are both inhibitory receptors of IGSF8 on NK or T cells, and the D1 domain of IGSF8 is the key domain mediating these receptor interactions.

### Example 18 Recombinant IGSF8 Specifically Binds to Domain 2 of KIR3DL1/2

This example demonstrates that Domain 2 of the KIR3DL1/2 is crucial for IGSF8 binding.

Like IGSF8, the KIR3DL1/2 receptors also have several Ig-like C2 topological extracellular domains. See domain organization below.

| | Amino Acid | |
|---|---|---|
| KIR3DL1/2 Proteins | Position | Description |
| Signal Peptide | 1-21 | — |
| Topological Domain 1 (D1) | 42-102 | Ig-like C2 domain |
| Topological Domain 2 (D2) | 137-202 | Ig-like C2 domain |
| Topological Domain 3 (D3) | 237-300 | Ig-like C2 domain |
| Transmembrane Domain (TM) | 341-360 | — |
| Cytoplasmic Tail | 361-444/455 | — |

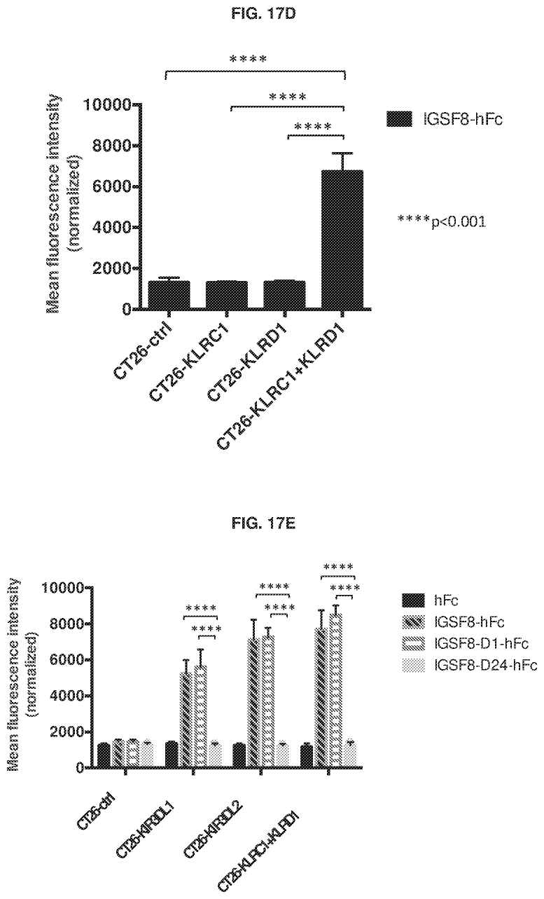

As shown in FIG. 18A, KIR3DL1 and KIR3DL2 share very similar topological structures. Different truncated forms of the KIR3DL1/2 genes were cloned and constructed into a lentiviral vector (see FIG. 17A), followed by lentiviral transduction of the mouse CT26 cells and testing IGSF8 binding with biotin-labelled recombinant IGSF8 protein. The mean fluorescence intensity (MFI) of IGSF8 binding to different CT26 cells were normalized by the expression of thy1.1 tag on different CT26 cells via flow cytometry.

Selectivity of IGSF8 binding to Domain 2 (D2) of KIR3DL1 and KIR3DL2 proteins was demonstrated, since binding of IGSF8 to the D12 construct containing the two terminal KIR domains was equal to that of the full-length KIR3DL1/2 proteins, but no binding was observed between IGSF8 and the D1/D3 construct containing only the terminal D1/D3 domain (FIG. 18B).

This data indicates that the middle D2 domain of KIR3DL1/2 is the major (if not only) functional domain to be bound by IGSF8. Thus, targeting the middle D2 domain of KIR3DL1/2 by various types of inhibitors (e.g., small molecule, antibody, peptide or nucleic acid) can modulate (inhibit) NK cell-based immunotherapy.

### Example 19 S165, I171, and M186 are Three Key Amino Acids for Effecting KIR3DL1/2 and IGSF8 Blockade

This example demonstrates that three residues in KIR3DL1/2 play important roles for interacting with IGSF8, and may be key to design inhibitors (small molecule, antibody, peptide or nucleic acid, etc.) that block IGSF8 binding to KIR3DL1/2.

Although KIR receptors share strong sequence homology within the KIR family, data in Example 17 demonstrates that IGSF8 selectively binds to only two of the many KIR receptors—KIR3DL1 and KIR3DL2, suggesting that IGSF8 binding to specific KTR receptors may be mediated by non-conserved residues in KIR3DL1/2.

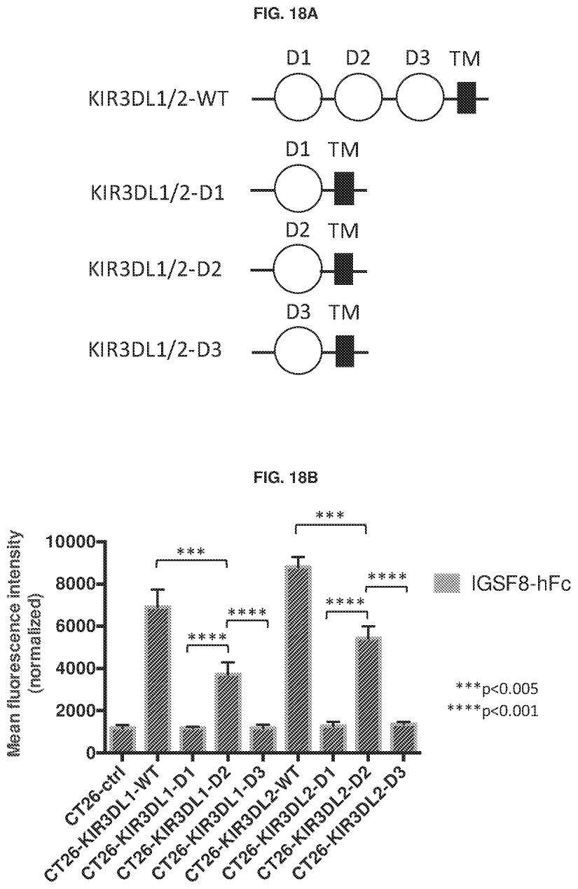

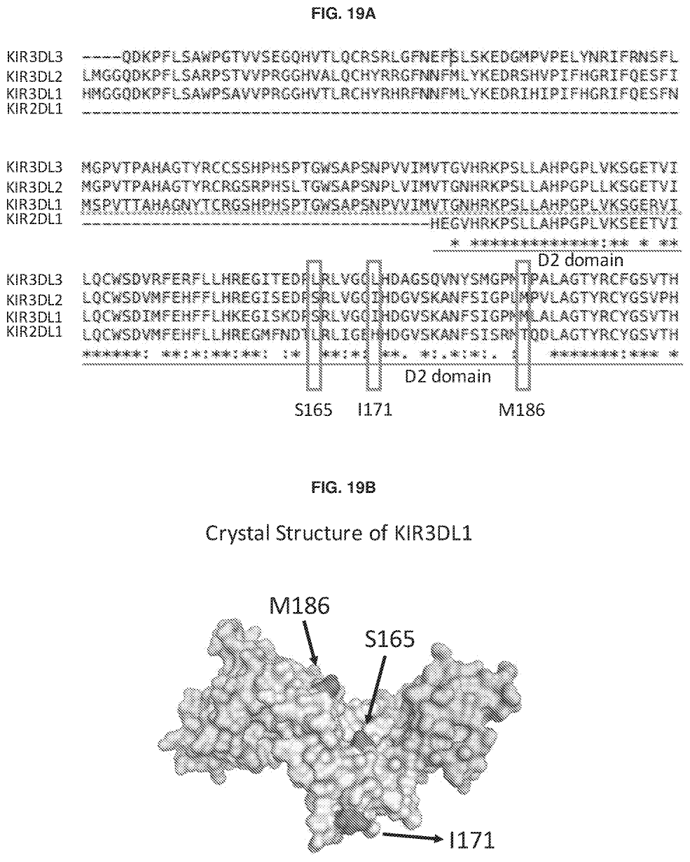

Thus a multiple sequence alignment of the KIR proteins was conducted, and the results were shown in FIG. 19A. In addition, the crystal structure of the KIR3DL1 (3VH8) protein from the PDB database was obtained (FIG. 19B). Based on these information and further mutation screening for binding, two there curial amino acids (S163, I171, and M186) were successfully identified in KIR3DL1/2 that play important roles for IGSF8 binding. Both All of the S165A, I171A mutation and the M186A mutation in KIR3DL1/2 remarkably affected IGSF8 binding to the KIR3DL1/2 receptors (FIG. 20).

This data illustrates that these three residues are important for IGSF8 binding, and may hold the key to designing inhibitor for block KIR3DL1/2 and IGSF8 interaction.

### Example 20 Characterization of Anti-IGSF8 Monoclonal Antibodies (mAbs)

A number of monoclonal antibodies against IGSF8 were generated in silico using a proprietary computer-assisted design algorithm, which generates sequences of fully human monoclonal antibodies against different targets, partly by taking advantage of the available sequences form the human (B cell receptor) BCR repertoire. This technology enables speedy design of numerous monoclonal antibodies against a given functional domain of a target antigen. For example, more than a hundred human monoclonal antibodies against human IGSF8 were generated by this method. After the sequences of the heavy chains and the light chains of these antibodies were obtained, monoclonal antibodies based on these sequences were expressed using CHO cells, and were further screened for high affinity binders to either the full-length IGSF8 or the D1 domain of IGSF8 using surface plasmon resonance (SPR) by Biacore T100. The antibodies with binding affinity (K_{D}) of less than 50 nM were chosen for further testing, for their abilities to bind IGSF8 expressed on cell surface by fluorescence-activated cell sorting (FACS). Ability to bind cell surface-expressed target antigen (i.e., IGSF8 in this case) is an indication of of more authentic binding compared to binding measured by Biacore.

For antibody binding assays based on cell-expressed antigens, 100,000 cells overexpressing the antigen (either the full-length IGSF8 or the D1 domain thereof) were washed with PBS buffer containing 2% FBS, and then incubated with 100 μL of antibodies or IgG of different concentrations for 30 minutes on ice. Cells were then washed twice with wash buffer, and incubated with 100 μL of 1:100 anti-human Fc-PE antibody for 15 minutes on ice. Cells were washed twice with wash buffer and analyzed on a FACS analyzer (CytoFlex, Beckman).

Among the 100 or so sequences tested, 10 different monoclonal antibodies were found to be able to bind to both the D1 domain and the full-length IGSF8 with a K_{D} value of less than 50 nM based on Biacore T100 measurement (Table 1).

TABLE 1
SPR test by Biacore T100

| | K_{D} (nM) |
|---|---|
| mAb-B34 (C30) | 13.8 |
| mAb-B46 (C31) | 7.29 |
| mAb-B104 (C32) | 25.9 |
| mAb-1C2 (C33) | 33.2 |
| mAb-1D7 (C34) | 2.12 |
| mAb-1B1 (C35) | 2.06 |
| mAb-1B4 (C36) | 4.57 |
| mAb-3F12(C37) | 22.5 |
| mAb-2B4 (C38) | 3.83 |
| mab-8G4 (C39) | 34.4 |

However, three of these high affinity binders based on Biacore measurement had weak or substantially no binding to the cell surface-expressed IGSF8, and were thus removed from further functional analysis.

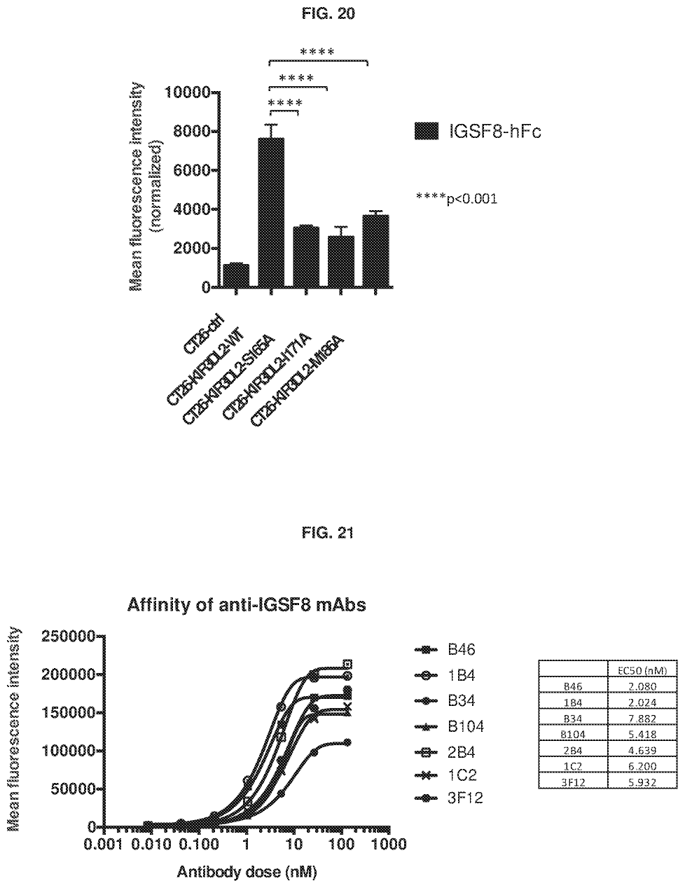

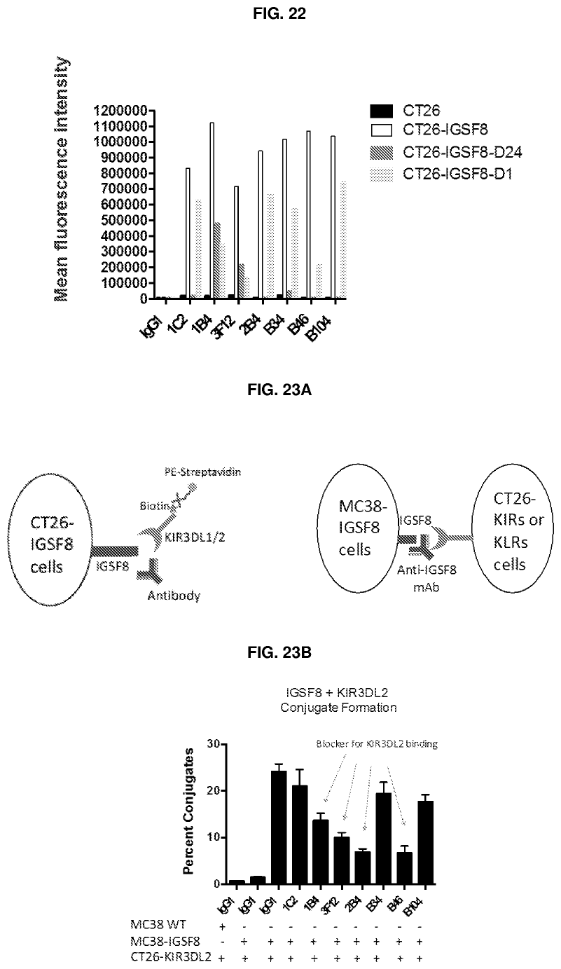

The remaining seven antibodies were shown with strong (1-10 nM level) binding to human IGSF8 proteins expressed on CT26 cell surface. More importantly, and consistent with the data in Example 16, all of these anti-IGSF8 antibodies were also shown to specifically bind to the D1 domain of IGSF8 (FIGS. 21 and 22).

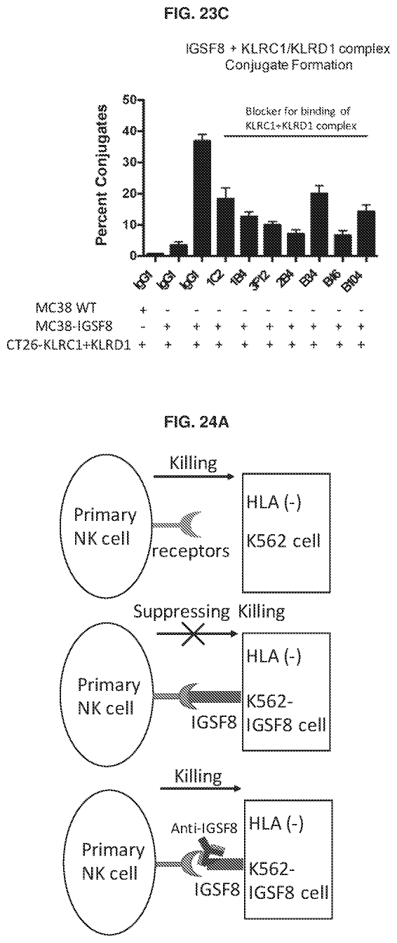

A competition assay (FIG. 23A) was then designed to test whether these seven mAbs block IGSF8 binding to the KIR3DL1/2 or the KLRC1&D1 heterodimer receptors. MC38 cells (a cell line derived from C57BL6 murine colon adenocarcinoma cells from NIH) with forced expression of IGSF8 protein on cell surface by lentiviral delivery of a human IGSF8-expression construct were labeled with CFSE (Carboxyfluorescein succinimidyl ester, Thermo). CT26 cells with forced expression of either KIR3DL1/2 or KLRC1&D1 were labeled with CellTrace Far Red (Thermo). The MC38 cells were then mixed at a 1:1 ratio with the CT26 cells for about an hour at room temperature. The formation of MC38 and CT26 cell/cell conjugates was assessed by flow cytometry.

Based on this analysis, it was shown that the 1B4, 3F12, 2B4 and B46 mAb can compete with IGSF8 binding to KIR3DL2 on MC38 cells (FIG. 23B), because the number of MC38 and CT26 cell/cell conjugates were remarkably reduced. Meanwhile, all of the seven mAbs can block IGSF8 binding KLRC1&D1 on MC38 cells (FIG. 23C). These data suggest that all of these mAbs targeting the D1 domain of IGSF8 functionally block the binding of IGSF8 to its cell surface receptors on NK or T cells.

### Example 21 Treatment with B46 and B104 mAbs in K562-NK Co-culture Restored NK Cell Cytolytic Activity

This example demonstrates that anti-IGSF8 monoclonal antibody can restore cytolytic activity of NK cells suppressed by IGSF8.

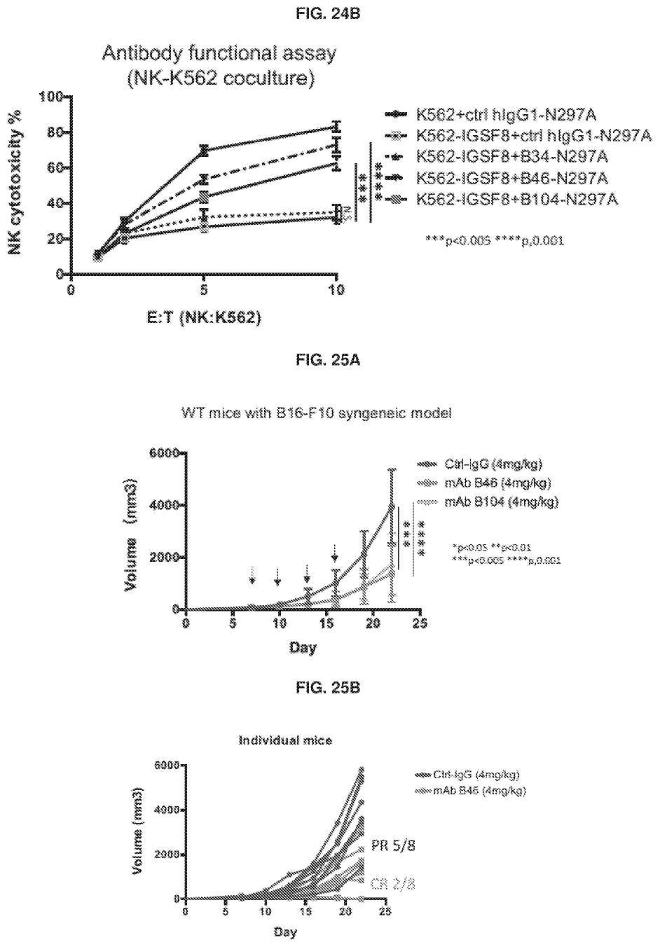

Functional assays shown in FIG. 24A were designed to further screen functional molecules against IGSF8 or KIR3DL1/2. In Example 4, it was shown that forced expression of IGSF8 on K562 cells by lentiviral delivery mostly protected the K562 cells from primary NK cell-mediated killing. Here, the protective effect conferred by IGSF8 expression were reversed by treatment with anti-IGSF8 mAbs in this co-culture model.

To exclude the confounding factor of IgG1-Fc mediated Antibody-Dependent Cellular Cytotoxicity (ADCC) in the experiment, a N297A mutated IgG1-Fc, which has been known to have largely lost the ADCC effect of human IgG1, was used in this assay. As shown in FIG. 24B, treatment with the B46 and the B104 mAb significantly restored cytolytic activity of NK cells.

### Example 22 the B46 and B104 mAbs Showed In Vivo Anti-Tumor Efficacy

Since both the B46 and the B104 mAbs can bind to human as well as mouse IGSF8, their abilities to inhibit IGSF8-KIR3DL1/2 binding in vivo were tested using the B16-F10 syngeneic mouse model.

In particular, about one million B16-F10 melanoma cells were injected subcutaneously into wild type (WT) C57BL/6 mice. Three groups of mice (n=8 mice per group) were then treated with one of two anti-IGSF8 mAbs, and one isotype-matched control antibody, respectively, at a dose of about 4 mg/kg, from Day 7 post tumor cell innoculation, every 3 days, for a total of four doses, by tail vein injection. Data were presented as mean s.e.m.

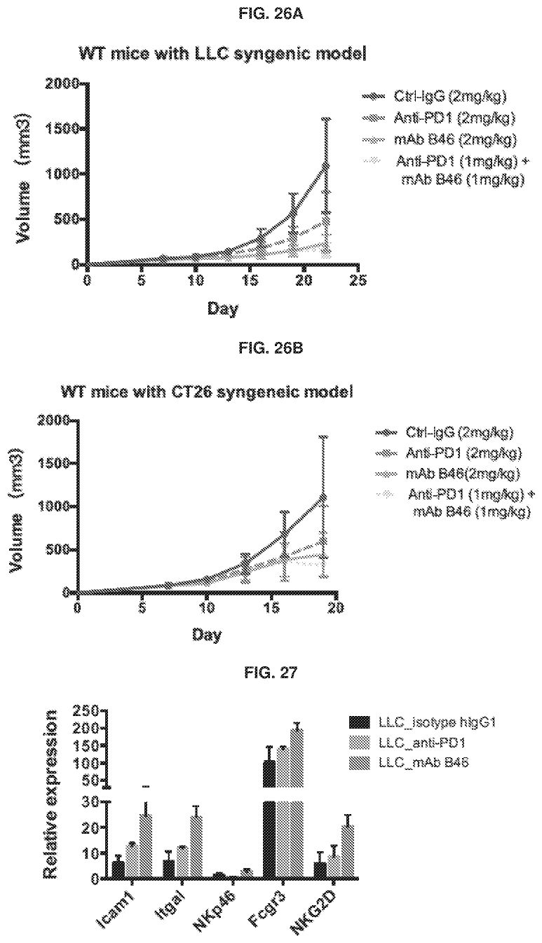

As shown in FIG. 25A, treatment of the B16 tumors with the B46 and the B104 mAbs significantly and dramatically reduced tumor growth compared to isotype-matched control. More strikingly, two of the eight mice treated with the B46 mAb achieved complete response (CR) with no detectable tumor after the treatment (FIG. 25B).

### Example 23 Synergistic Anti-tumor Effects by Administering Anti-IGSF8 Antibody and Anti-PD-1 Antibody to a Representative Mouse Model

Checkpoint inhibitors are well established treatments for selected human malignancies. A major research effort has been for finding other treatments which may enhance immunological activity of this pathway. Given the characteristics, IGSF8 was investigated for its potential for combination with a T cell-based immunotherapy agent—anti-PD-1 antibody, in Example 11. Results from that example demonstrated that the anti-IGSF8 monoclonal antibodies and anti-PD-1 antibody exhibited synergistic effect in inhibiting B16-F10 melanoma tumor growth in vivo in a syngenic mouse model.

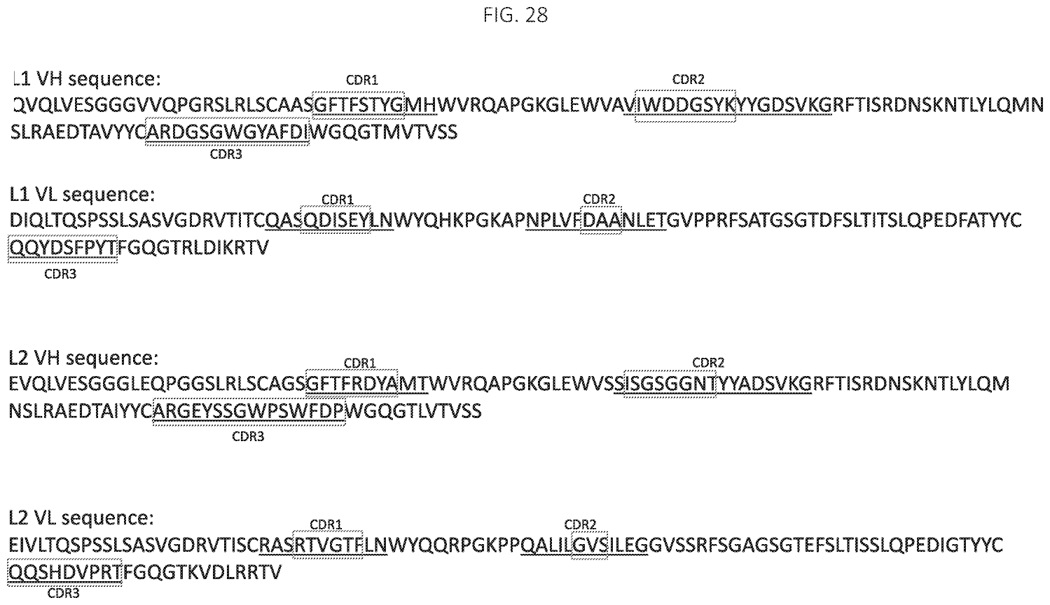

This example provides further evidence to demonstrate enhanced synergistic activity due to combining IGSF8 monoclonal antibody and anti-PD-1 monoclonal antibodies, using both CT-26 (colon carcinoma) and LLC (Lewis Lung Carcinoma) cancer cell lines (FIGS. 26A & 26B).

Specifically, about one million CT26 or LLC cells were injected subcutaneously into wild type (WT) C57BL/6 mice. Mice were then treated, by tail vein injection, with one of four antibodies or antibody combinations: IgG control at a dose of 2 mg/kg; anti-PD-1 antibody at a dose of 2 mg/kg; anti-IGSF8 antibody B46 at a dose of 2 mg/kg; or a combination of anti-PD-1 antibody at half the dose (1 mg/kg) and anti-IGSF8 antibody B46 at half the dose (1 mg/kg). The first doses were administered on Day 6 post tumor cell innoculation, and the subsequent doses were administered every 3 days, for four doses in total. Data were presented as mean±s.e.m. (n=8 mice per group).

The combination of an anti-IGSF8 antibody and anti-PD-1 antibody exhibited synergistic effect in inhibiting the B16 melanoma growth in vivo, in that the combination therapy, when administered at a 50% dose (1 mg/kg) for each component of the combination, was statistically significantly superior to (1) the anti-IGSF8 antibody B46 alone at twice the dose (2 mg/kg) (p<0.01), (2) the commercial anti-PD-1 antibody (Clone 29F.1A12, BioXcell) alone at twice the dose (2 mg/kg) (p<0.005), and (3) IgG control (p<0.001).

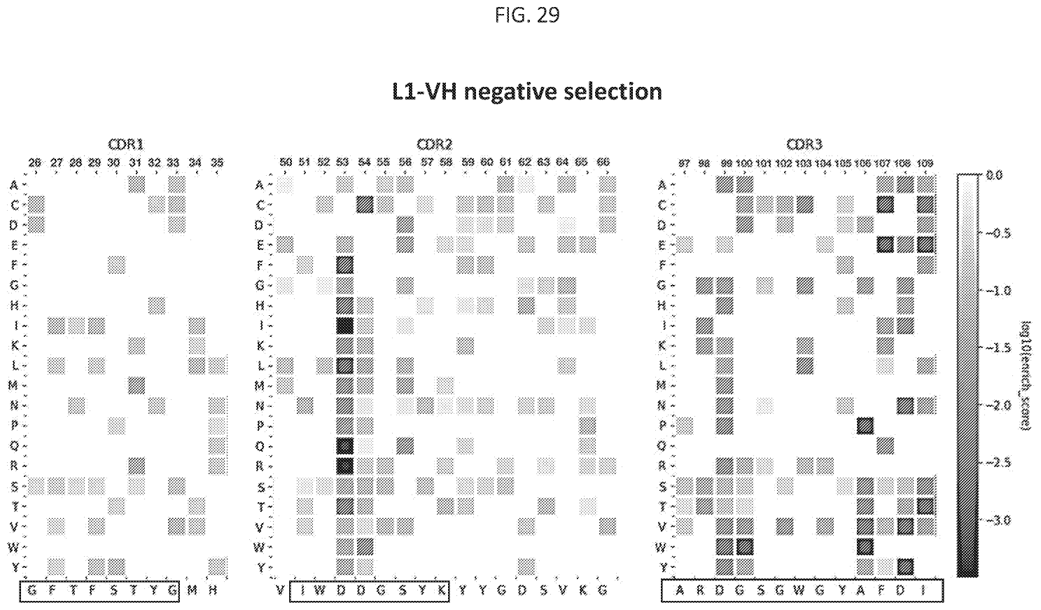

Moreover, the gene markers of activated NK cells were remarkably increased in the LLC tumors treated with the anti-IGSF8 B46 mAb, as compared to the tumors treated with the anti-PD-1 mAb or IgG control (FIG. 27), suggesting that there is more infiltration of activated NK cells in the LLC tumors upon anti-IGSF8 B46 treatment.

These data are consistent with the previous finding, which showed that loss or inhibition of IGSF8 reprogramed the tumor microenvironment, and re-activated the NK cell-mediated anti-tumor function in vivo.

While not wishing to be bound by any particular theory, this mechanism helps to illucidate a potential reason concerning the synergistic anti-tumor effect exhibited by the combination of anti-IGSF8 treatment with anti-PD-1 treatment.

These results indicate that combining IGSF8 antagonists with antagonists of either T cell-based immune checkpoint inhibition or target therapy based targets may be effective for various cancer treatments.

### Example 24 Generation of IGSF8 Fully Human Monoclonal Antibodies

A number of monoclonal antibodies against IGSF8 were generated in silico using a proprietary computer-assisted design algorithm, which generates sequences of fully human monoclonal antibodies against different targets, partly by taking advantage of the available sequences from the human B cell receptor (BCR) repertoire. This technology enables speedy design of numerous monoclonal antibodies against a given functional domain of a target antigen.

Using this approach, more than a hundred human monoclonal antibodies against human IGSF8 were generated. After the sequences of the heavy chains and the light chains of these antibodies were obtained in silico, monoclonal antibodies based on these sequences were expressed using CHO cells, and were further screened for high affinity binders to either the full-length IGSF8, or the D1 domain of IGSF8 using surface plasmon resonance (SPR) by Biacore T100. Antibodies with high binding affinity (K_{D}) of less than 50 nM were chosen for further testing for their abilities to bind IGSF8 expressed on cell surface, by fluorescence-activated cell sorting (FACS). Ability to bind cell surface-expressed target antigen (i.e., IGSF8 in this case) is an indication of more authentic binding compared to binding measured by Biacore.

Among the 100 or so sequences tested, two antibodies (L1, L2) were selected for further investigation. The sequences of L1 and L2 are listed under L1-01 in Table D and L2-01 in Table G, respectively.

### Example 25 Mutagenesis Study Targeting the CDRs of Ab Heavy and Light Chains

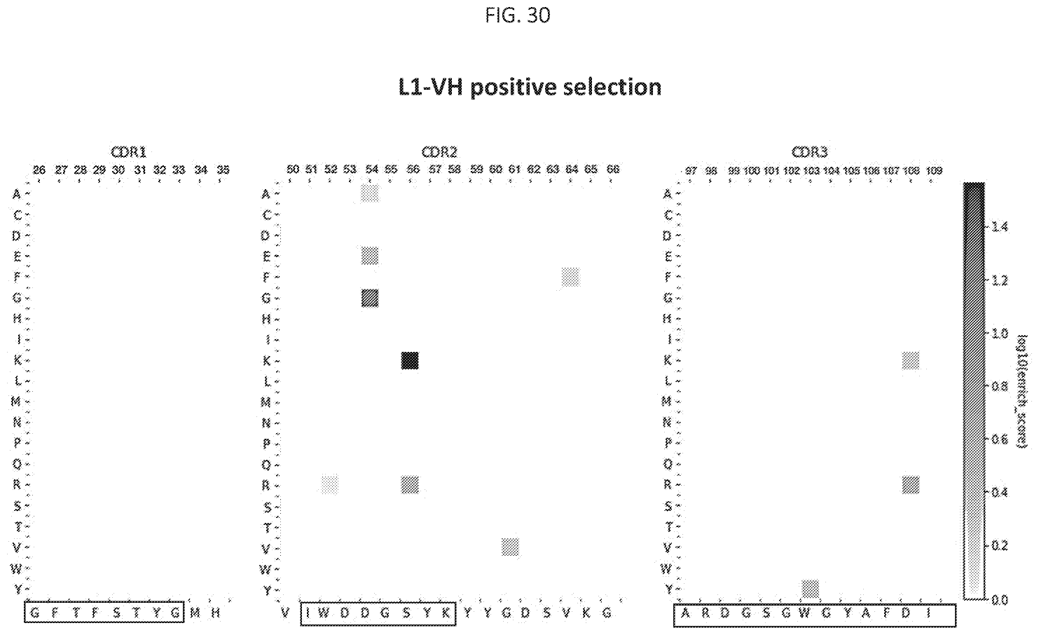

To study the amino acids within the CDR regions of the antibody heavy and light chains that contribute to the binding to the IGSF8 target antigen, the CDR1, 2, 3 of the heavy and light chains from the L1 and L2 antibodies (see Example 24) were selected for randomization by using the degenerate codons NNK (FIG. 28).

Six sub-libraries that targets each CDR region were first generated, followed by two rounds of phage display screening. The libraries before and after phage panning were further evaluated by the Illumina high-throughput sequencing platforms. For a comprehensive analysis of positive or negative selection of each mutant that affects binding to IGSF8, the fold change of frequencies of each mutation within each position of CDRs of heavy and light chains were calculated and plotted (FIGS. 29-36). Note that for each residue in each CDR region, all 19 possible mutations were tested.

The heatmap showed that the key positions within CDRs of heavy and light chains that are important for binding to IGSF8, for instance, D53, D54, D99, G100G, A106, F107, D108, and 1109 in L1-VH are enriched in the negative selection, indicating that mutants in this region/these residues decreased antibody affinity. See FIG. 29.

For further validation of the mutants that were determined to be essential for binding (based on the above negative selection), eight mutants for the L1 antibody, and ten mutants for the L2 antibody were chosen for further expression in CHO cells, and their binding affinities to IGSF8 were tested by surface plasmon resonance (SPR). Tables 1 and 3 showed that the mutants decreased the affinity.

The affinity of L1 and L2 antibodies were further optimized based on the positive selection of the mutants. 32 sequences of L1 and 9 sequences of L2 were tested and confirmed to dramatically increase antibody affinity (Table 2 and 4).

The binding affinities of the L1 and L2 antibodies to IGSF8 recombinant protein were determined by surface plasmon resonance (SPR). Briefly, anti-human Fab antibody was imobilized on acarboxyl-derivatized SPR chip surface, and the antibodies were captured on the resulting surface at 5 μg/ml for 30 seconds. The IGSF8-hFc protein at various concentrations (0 nM, 3.7 nM, 11.1 nM, 33.3 nM, 100 nM, and 300 nM) was then flowed over the surface and allowed to bind to the anti-IGSF8 antibodies during the dissociation phase. Data was fitted using a 1:1 binding model, and representative results are summarized in Tables 1-4.

TABLE 1
Representative mutations on heavy or light
chain of L1 that decreased affinity

| | ka (1/Ms) | kd (1/s) | K_{D} (M) |
|---|---|---|---|
| L1-01 | 1.97E+05 | 2.47E−03 | 1.26E−08 |
| L1VH-W52L | 1.93E+04 | 1.65E−02 | 8.55E−07 |
| L1VH-D53Y | 4.61E+04 | 1.68E−02 | 3.65E−07 |
| L1VH-D54T | 2.29E+03 | 1.31E−03 | 5.71E−07 |
| L1VH-D99N | 1.78E+04 | 2.46E−03 | 1.38E−07 |
| L1VL-P46L | 2.75E+04 | 3.41E−03 | 1.24E−07 |
| L1VL-Q24H | 5.37E+04 | 7.75E−03 | 1.44E−07 |
| L1VL-V48D | 3.24E+04 | 2.30E−02 | 7.08E−07 |
| L1VL-Y91F | 4.50E+03 | 1.45E−03 | 3.21E−07 |

TABLE 2
Antibodies derived from L1 with increased affinity

| | ka (1/Ms) | kd (1/s) | KD (M) |
|---|---|---|---|
| L1-01 | 1.97E+05 | 2.47E−03 | 1.26E−08 |
| L1-02 | 3.00E+05 | 1.27E−03 | 4.22E−09 |
| L1-03 | 2.93E+05 | 1.72E−03 | 5.88E−09 |
| L1-04 | 3.00E+05 | 2.20E−03 | 7.33E−09 |
| L1-05 | 2.41E+05 | 1.48E−03 | 6.17E−09 |
| L1-06 | 4.53E+05 | 1.04E−03 | 2.30E−09 |
| L1-07 | 3.32E+05 | 4.45E−04 | 1.34E−09 |
| L1-08 | 4.37E+05 | 2.53E−03 | 5.78E−09 |
| L1-09 | 4.08E+05 | 2.23E−03 | 5.47E−09 |
| L1-10 | 3.01E+05 | 1.96E−03 | 6.52E−09 |
| L1-11 | 3.39E+05 | 2.40E−03 | 7.08E−09 |
| L1-12 | 4.61E+05 | 1.42E−03 | 3.08E−09 |
| L1-13 | 4.43E+05 | 2.16E−03 | 4.88E−09 |
| L1-14 | 3.81E+05 | 2.44E−03 | 6.41E−09 |
| L1-15 | 3.62E+05 | 1.55E−03 | 4.28E−09 |
| L1-16 | 3.33E+05 | 1.11E−03 | 3.33E−09 |
| L1-17 | 3.18E+05 | 1.20E−03 | 3.78E−09 |
| L1-18 | 2.85E+04 | 2.68E−04 | 9.40E−09 |
| L1-19 | 1.54E+05 | 7.05E−04 | 4.58E−09 |
| L1-20 | 3.29E+05 | 7.02E−06 | 2.14E−11 |
| L1-21 | 4.06E+05 | 2.72E−03 | 6.71E−09 |
| L1-22 | 1.23E+05 | 3.63E−04 | 2.94E−09 |
| L1-23 | 5.06E+05 | 1.19E−06 | 2.35E−12 |
| L1-24 | 4.50E+05 | 3.06E−06 | 6.80E−12 |
| L1-25 | 4.92E+05 | 2.20E−03 | 4.46E−09 |
| L1-26 | 5.03E+05 | 4.97E−04 | 9.87E−10 |
| L1-27 | 4.44E+05 | 8.35E−04 | 1.88E−09 |
| L1-28 | 3.67E+05 | 4.28E−07 | 1.17E−12 |
| L1-29 | 3.81E+05 | 4.25E−06 | 1.12E−11 |
| L1-30 | 4.72E+05 | 3.03E−06 | 6.42E−12 |
| L1-31 | 2.10E+05 | 7.80E−07 | 3.71E−12 |
| L1-32 | 3.12E+05 | 6.56E−06 | 2.10E−11 |
| L1-33 | 2.76E+05 | 8.01E−06 | 2.90E−11 |

TABLE B
Permissible amino acid substitutions in CDRs of the L1 VH
that do not negatively affect binding of L1 (see FIG. 29)

| | | |
|---|---|---|
| | Original | |
| Position | amino acid | Permissible amino acids |

CDR1

| | | |
|---|---|---|
| 26 | G | A, E, F, G, H, I, K, L, M, N, P, Q, R, T, V, W or Y |
| 27 | F | A, C, D, E, F, G, H, K, M, N, P, Q, R, T, or W |
| 28 | T | A, C, D, E, F, G, H, K, L, M, P, Q, R, T, V, W or Y |
| 29 | F | A, C, D, E, F, G, H, K, M, N, P, Q, R, T, or W |
| 30 | S | A, C, D, E, G, H, I, K, L, M, N, Q, R, S, V, or W |
| 31 | T | C, D, E, F, G, H, I, L, N, P, Q, T, V, W or Y |
| 32 | Y | A, D, E, F, G, I, K, L, M, P, Q, R, S, T, V, W or Y |
| 33 | G | E, F, G, H, I, K, L, M, N, P, Q, R, T, W or Y |
| 34 | M | A, C, D, E, F, G, H, M, N, P, Q, R, S, W or Y |
| 35 | H | A, C, D, E, F, G, H, I, K, M, S, T, V, or W |

CDR2

| | | |
|---|---|---|
| 50 | V | C, D, F, H, I, K, N, P, Q, R, S, T, V, W or Y |
| 51 | I | A, C, D, E, G, H, I, K, L, M, P, Q, R, W or Y |
| 52 | W | A, D, E, F, H, I, K, M, N, P, Q, R, T, V, W or Y, e.g., R |
| 53 | D | C or D |
| 54 | D | A, D, E, F or G, e.g., G, E, or A, most preferably G |
| 55 | G | D, E, F, G, H, I, K, L, M, N, P, Q, T, W or Y |
| 56 | S | C, F, H, K, P, R, S, T, W or Y, e.g., K or R, most preferably K |
| 57 | Y | A, D, E, F, G, I, K, L, M, P, Q, R, T, V, W or Y |
| 58 | K | A, C, D, F, G, H, I, K, L, P, Q, S, V, W or Y |
| 59 | Y | A, G, I, L, M, P, R, V, W or Y |
| 60 | Y | A, E, G, I, K, L, M, P, Q, R, T, V, W or Y |
| 61 | G | E, F, G, H, I, K, L, M, N, P, Q, T, V, W or Y, e.g., V |
| 62 | D | C, D, F, I, K, L, M, P, Q, R, S, T or W |
| 63 | S | A, D, E, F, H, K, L, M, P, Q, S, V, W or Y |
| 64 | V | C, F, K, M, N, P, Q, R, S, T, V, W or Y, e.g., F |
| 65 | K | A, C, D, F, G, H, K, L, M, S, V, W or Y |
| 66 | G | E, F, G, H, I, K, L, M, N, P, Q, S, T, W or Y |

CDR3

| | | |
|---|---|---|
| 97 | A | A, C, D, F, G, H, I, K, L, M, N, Q, R, W or Y |
| 98 | R | A, C, D, E, F, H, L, M, N, P, Q, R, V, W or Y |
| 99 | D | C, D, F, I or Q |
| 100 | G | E, F, G, H, I, K, L, M, N, P or Q |
| 101 | S | A, D, E, F, H, I, K, L, M, P, Q, S, T, V, W or Y |
| 102 | G | A, E, F, G, H, I, K, L, M, N, P, Q, R, T, W or Y |
| 103 | W | A, D, E, F, H, I, M, N, P, Q, S, T, V, W or Y, e.g., Y |
| 104 | G | A, C, D, F, G, H, I, K, L, M, N, P, Q, S, T, W or Y |
| 105 | Y | A, E, G, I, K, L, M, P, Q, R, T, V, W or Y |
| 106 | A | A, C, E, F, H, I, K, L, M, N, Q or R |
| 107 | F | D, F, G, H, M, N, P, R, T or W |
| 108 | D | C, D, F, K, L, M, P, Q, R or W, e.g., R or K |
| 109 | I | G, H, I, K, M, P, Q, R, W or Y |

In the table above, residues after “e.g.” are residue changes that enhance binding compared to the original residue.

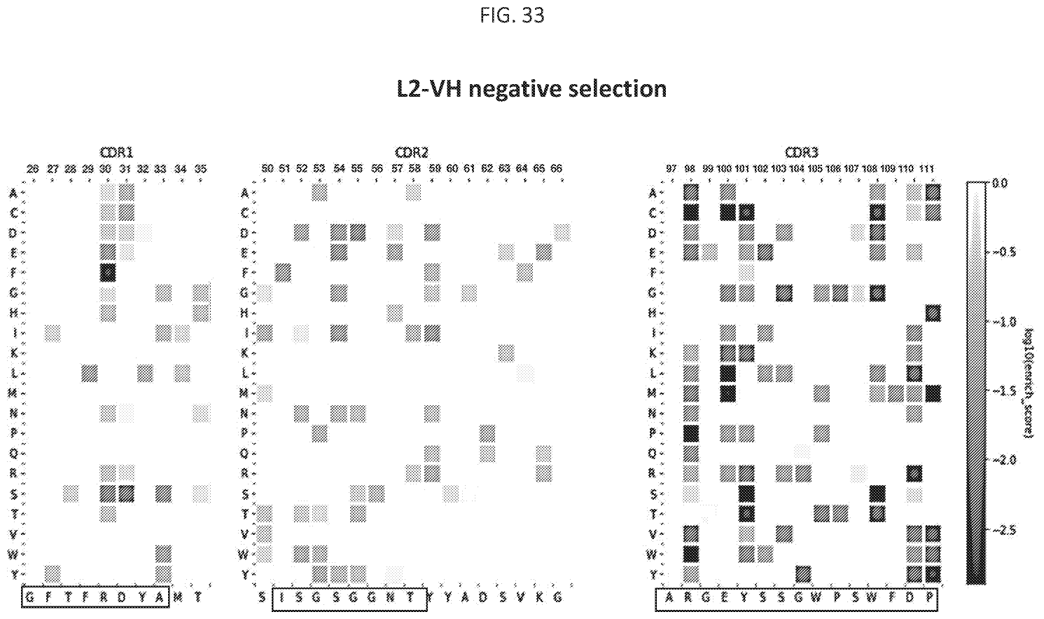

TABLE C
Permissible amino acid substitutions in CDRs of the L1 VL
that do not negatively affect binding of L1 (see FIG. 31)

| | | |
|---|---|---|
| | Original | |
| Position | amino acid | Permissible amino acids |

CDR1

| | | |
|---|---|---|
| 24 | Q | A, C, D, E, F, G, I, L, M, N, P, Q, S, T, V, W or Y |
| 25 | A | A, C, D, F, G, H, I, K, L, M, N, P, Q, R, T, V, W or Y |
| 26 | S | A, D, E, F, H, K, L, M, N, P, Q, S, T, V, W or Y |
| 27 | Q | A, C, D, E, F, G, I, K, L, M, N, Q, S, T, V, W or Y |
| 28 | D | A, C, D, E, F, H, I, K, L, M, N, P, Q, R, T, V or W |
| 29 | I | A, C, D, E, F, G, H, I, K, M, P, Q, R, V, W or Y |
| 30 | S | C, D, E, F, G, K, L, M, R, S, T, V, W or Y, e.g., E, G, K, M, T, V, or W |
| | E | C, D, E, F, G, H, I, L, M, P, Q, S, T, V, W or Y, e.g., D, F, G, L, M, P, Q, |
| | | S, T, V, W, or Y |
| 32 | Y | A, C, F, G, H, I, Q, S, T, W or Y, e.g., A, C, G, Q, S, T, or W, most |
| | | preferably W |
| 33 | L | A, C, D, E, F, G, H, K, L, M, N, P, Q, R, S, T, W or Y |
| 34 | N | A, C, D, E, F, G, H, L, M, N, P, Q, R, S, T, V, W or Y |

CDR2

| | | |
|---|---|---|
| 45 | N | A, C, D, E, F, G, K, L, M, N, P, Q, R, S, V, W or Y |
| 46 | P | A, C, D, E, F, G, I, K, M, N, P, Q, R, S, V, W or Y |
| 47 | L | A, C, D, E, F, G, H, I, K, L, N, S, T, V, W or Y |
| 48 | V | A, C, E, H, K, L, M, N, P, Q, R, S, T, V, W or Y |
| 49 | F | A, D, E, F, G, H, K, N, P, Q, R, T or W |
| 50 | D | A, C, D, F, G, H, N, R or S, e.g., A, G, H, N, R or S, most preferably G |
| 51 | A | A, C, D, I, K, S or T, e.g., D, S or T, most preferably S |
| 52 | A | A, C, D, E, F, H, I, N, P, S, T, V or W, e.g., A, D, E, F, H, N, P, V, or W, |
| | | most preferably P |
| 53 | N | A, C, E, G, H, I, L, M, N, P, Q, R, V, W or Y |
| 54 | L | A, D, E, F, G, H, I, K, L, N, P, Q, R, T or Y |
| 55 | E | C, E, F, H, I, M, N, P, R, S, T, V, W or Y |
| 56 | T | C, D, E, F, G, H, L, M, N, Q, T, V, W or Y |

CDR3

| | | |
|---|---|---|
| 89 | Q | A, C, D, E, F, G, I, M, N, P, Q, S, T, V, W or Y |
| 90 | Q | A, C, D, E, F, G, I, M, N, P, Q, S, T, V, W or Y |
| 91 | Y | A, C, D, E, G, I, K, L, M, N, P, Q, R, T, V, W or Y |
| 92 | D | D, E, F, P, Q or Y, e.g., E, Q, or Y |
| 93 | S | G, K, L, M, N, P, Q, R or S e.g., G or K |
| 94 | F | C, D, E, F, H, I, L, M, N, P, Q, S, T, V or Y, e.g., D, E, L, M, N, Q, T, or V |
| 95 | P | A, C, D, E, F, G, I, K, L, M, N, P, Q, R, V, W or Y |
| 96 | Y | A, E, F, G, I, K, M, N, P, Q, R, T, V, W or Y |
| 97 | T | C, D, E, F, G, H, I, K, L, M, N, Q, R, T, V, W or Y |

In the table above, residues after “e.g.” are residue changes that enhance binding compared to the original residue.

+0 TABLE D
Sequences of selected antibodies derived from L1 with increased affinity

| Antibody | | Sequence | SEQ ID NO: |
|---|---|---|---|
| L1-01 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 673 |
| | | PNPLVFDAANLETGVPPRFSATGSGTDFSLTITSLQPEDFATYY | |
| | | CQQYDSFPYTFGQGTRLDIK | |
| | VL CDR1 | QDISEY | SEQ ID NO: 611 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-02 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISKPLNWYQHKPGKA | SEQ ID NO: 674 |
| | | PNPLVFDAANLETGVPPRFSATGSGTDFSLTITSLQPEDFATYY | |
| | | CQQYDSFPYTFGQGTRLDIK | |
| | VL CDR1 | QDISKP | SEQ ID NO: 612 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-03 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 675 |
| | | PNPLVFRSPNLETGVPPRFSATGSGTDFSLTITSLQPEDFATYYC | |
| | | QQYDSFPYTFGQGTRLDIK | |
| | VL CDR1 | QDISEY | SEQ ID NO: 611 |
| | VL CDR2 | RSP | SEQ ID NO: 624 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-04 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 676 |
| | | PNPLVFGSPNLETGVPPRFSATGSGTDFSLTITSLQPEDFATYYC | |
| | | QQYDSFPYTFGQGTRLDIK | |
| | VL CDR1 | QDISEY | SEQ ID NO: 611 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-05 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDITKGLNWYQHKPGKA | SEQ ID NO: 677 |
| | | PNPLVFDAANLETGVPPRFSATGSGTDFSLTITSLQPEDFATYY | |
| | | CQQYDSFPYTFGQGTRLDIK | |
| | VL CDR1 | QDITKG | SEQ ID NO: 613 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-06 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDIGPWLNWYQHKPGK | SEQ ID NO: 678 |
| | | APNPLVFDAANLETGVPPRFSATGSGTDFSLTITSLQPEDFATY | |
| | | YCQQYDSFPYTFGQGTRLDIK | |
| | VL CDR1 | QDIGPW | SEQ ID NO: 614 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-07 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDIEWWLNWYQHKPGK | SEQ ID NO: 679 |
| | | APNPLVFDAANLETGVPPRFSATGSGTDFSLTITSLQPEDFATY | |
| | | YCQQYDSFPYTFGQGTRLDIK | |
| | VL CDR1 | QDIEWW | SEQ ID NO: 615 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-08 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDIGAYLNWYQHKPGKA | SEQ ID NO: 680 |
| | | PNPLVFDAANLETGVPPRFSATGSGTDFSLTITSLQPEDFATYY | |
| | | CQQYDSFPYTFGQGTRLDIK | |
| | VL CDR1 | QDIGAY | SEQ ID NO: 616 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-09 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 681 |
| | | PNPLVFARDNLETGVPPRFSATGSGTDFSLTITSLQPEDFATY | |
| | | YCQQYDSFPYTFGQGTRLDIK | |
| | VL CDR1 | QDISEY | SEQ ID NO: 611 |
| | VL CDR2 | ARD | SEQ ID NO: 626 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-10 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 682 |
| | | PNPLVFTSANLETGVPPRFSATGSGTDFSLTITSLQPEDFATYYC | |
| | | QQYDSFPYTFGQGTRLDIK | |
| | VL CDR1 | QDISEY | SEQ ID NO: 611 |
| | VL CDR2 | TSA | SEQ ID NO: 627 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-11 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDIEWALNWYQHKPGK | SEQ ID NO: 683 |
| | | APNPLVFDAANLETGVPPRFSATGSGTDFSLTITSLQPEDFATY | |
| | | YCQQYDSFPYTFGQGTRLDIK | |
| | VL CDR1 | QDIEWA | SEQ ID NO: 617 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-12 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 684 |
| | | PNPLVFGADNLETGVPPRFSATGSGTDFSLTITSLQPEDFATYY | |
| | | CQQYDSFPYTFGQGTRLDIK | |
| | VL CDR1 | QDISEY | SEQ ID NO: 611 |
| | VL CDR2 | GAD | SEQ ID NO: 628 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-13 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 685 |
| | | PNPLVFDAPNLETGVPPRFSATGSGTDFSLTITSLQPEDFATYY | |
| | | CQQYDSFPYTFGQGTRLDIK | |
| | VL CDR1 | QDISEY | SEQ ID NO: 611 |
| | VL CDR2 | DAP | SEQ ID NO: 629 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-14 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDISEYLNWYQHKPGKA | SEQ ID NO: 686 |
| | | PNPLVFSQPNLETGVPPRFSATGSGTDFSLTITSLQPEDFATYY | |
| | | CQQYDSFPYTFGQGTRLDIK | |
| | VL CDR1 | QDISEY | SEQ ID NO: 611 |
| | VL CDR2 | SQP | SEQ ID NO: 630 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-15 | VH | QVQLVESGGGVVQPGRSLRLSCAASGFTFSTYGMHWVRQAP | SEQ ID NO: 669 |
| | | GKGLEWVAVIWDDGSYKYYGDSVKGRFTISRDNSKNTLYLQM | |
| | | NSLRAEDTAVYYCARDGSGWGYAFDIWGQGTMVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTMVTVSS | SEQ ID NO: 609 |
| | VL | DIQLTQSPSSLSASVGDRVTITCQASQDIMKTLNWYQHKPGK | SEQ ID NO: 687 |
| | | APNPLVFDAANLETGVPPRFSATGSGTDFSLTITSLQPEDFATY | |
| | | YCQQYDSFPYTFGQGTRLDIK | |
| | VL CDR1 | QDIMKT | SEQ ID NO: 618 |
| | VL CDR2 | DAA | SEQ ID NO: 623 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPNPLVF | SEQ ID NO: 634 |
| | VL FR3 | NLETGVPPRFSATGSGTDFSLTITSLQPEDFATYYC | SEQ ID NO: 638 |
| | VL FR4 | FGQGTRLDIK | SEQ ID NO: 641 |
| L1-16 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQMTQSPSSLSASVGDRVTITCQASQDIGPWLNWYQQKPG | SEQ ID NO: 688 |
| | | KAPKLLIYGSPNLETGVPSRFSGSGSGTDFTFTISSLQPEDIATYY | |
| | | CQQYDSFPYTFGQGTKVEIK | |
| | VL CDR1 | QDIGPW | SEQ ID NO: 614 |
| | VL CDR2 | GSP | SEQ ID NO: 625 |
| | VL CDR3 | QQYDSFPYT | SEQ ID NO: 631 |
| | VL FR1 | DIQMTQSPSSLSASVGDRVTITCQAS | SEQ ID NO: 633 |
| | VL FR2 | LNWYQQKPGKAPKLLIY | SEQ ID NO: 635 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-17 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQMTQSPSSLSASVGDRVTITCQASQDIGPWLNWYQQKPG | SEQ ID NO: 689 |
| | | KAPKLLIYDAPNLETGVPSRFSGSGSGTDFTFTISSLQPEDIATYY | |
| | | CQQYDSFPYTFGQGTKVEIK | |
| | VL CDR1 | QDIGPW | SEQ ID NO: 614 |
| | VL CDR2 | DAP | SEQ ID NO: 629 |
| | VL CDR3 | QQYDSFPYT | SEQ ID NO: 631 |
| | VL FR1 | DIQMTQSPSSLSASVGDRVTITCQAS | SEQ ID NO: 633 |
| | VL FR2 | LNWYQQKPGKAPKLLIY | SEQ ID NO: 635 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-18 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQMTQSPSSLSASVGDRVTITCQASQDIGPWLNWYQQKPG | SEQ ID NO: 688 |
| | | KAPKLLIYGSPNLETGVPSRFSGSGSGTDFTFTISSLQPEDIATYY | |
| | | CQQYDSFPYTFGQGTKVEIK | |
| | VL CDR1 | QDIGPW | SEQ ID NO: 614 |
| | VL CDR2 | GSP | SEQ ID NO: 625 |
| | VL CDR3 | QQYDSFPYT | SEQ ID NO: 631 |
| | VL FR1 | DIQMTQSPSSLSASVGDRVTITCQAS | SEQ ID NO: 633 |
| | VL FR2 | LNWYQQKPGKAPKLLIY | SEQ ID NO: 635 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-19 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQMTQSPSSLSASVGDRVTITCQASQDISPYLNWYQQKPGK | SEQ ID NO: 690 |
| | | APKLLIYDAPNLETGVPSRFSGSGSGTDFTFTISSLQPEDIATYYC | |
| | | QQYDSFPYTFGQGTKVEIK | |
| | VL CDR1 | QDISPY | SEQ ID NO: 619 |
| | VL CDR2 | DAP | SEQ ID NO: 629 |
| | VL CDR3 | QQYDSFPYT | SEQ ID NO: 631 |
| | VL FR1 | DIQMTQSPSSLSASVGDRVTITCQAS | SEQ ID NO: 633 |
| | VL FR2 | LNWYQQKPGKAPKLLIY | SEQ ID NO: 635 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-20 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIEWWLNWYQQKPGK | SEQ ID NO: 691 |
| | | APKLLIYGSPNLETGVPSRFSGSGSGTDFTFTISSLQPEDIATYYC | |
| | | QQYDSFPYTFGQGTKVEIK | |
| | VL CDR1 | QDIEWW | SEQ ID NO: 615 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQQKPGKAPKLLIY | SEQ ID NO: 635 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-21 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPWLNWYQHKPGK | SEQ ID NO: 692 |
| | | APKPLIYGSPNLETGVPSRFSGSGSGTDFTFTISSLQPEDIATYYC | |
| | | QQYDSFPYTFGQGTKVEIK | |
| | VL CDR1 | QDIGPW | SEQ ID NO: 614 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLIY | SEQ ID NO: 636 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-22 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDISPWLNWYQHKPGK | SEQ ID NO: 693 |
| | | APKPLIYGSPNLETGVPSRFSGSGSGTDFTFTISSLQPEDIAT | |
| | | YYCQQYDSFPYTFGQGTKVEIK | |
| | VL CDR1 | QDISPW | SEQ ID NO: 620 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLIY | SEQ ID NO: 636 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-23 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPWLNWYQHKPGK | SEQ ID NO: 694 |
| | | APKPLVFGSPNLETGVPSRFSASGSGTDFTLTISSLQPEDFATYY | |
| | | CQQYDSFPYTFGQGTKVEIK | |
| | VL CDR1 | QDIGPW | SEQ ID NO: 614 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-24 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPYLNWYQHKPGKA | SEQ ID NO: 695 |
| | | PKPLVFGSPNLETGVPSRFSASGSGTDFTLTISSLQPEDFATYYC | |
| | | QQYDSFPYTFGQGTKVEIK | |
| | VL CDR1 | QDIGPY | SEQ ID NO: 621 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-25 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDISPWLNWYQHKPGK | SEQ ID NO: 696 |
| | | APKPLVFGSPNLETGVPSRFSASGSGTDFTLTISSLQPEDFATYY | |
| | | CQQYDSFPYTFGQGTKVEIK | |
| | VL CDR1 | QDISPW | SEQ ID NO: 620 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-26 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDISPYLNWYQHKPGKA | SEQ ID NO: 697 |
| | | PKPLVFGSPNLETGVPSRFSASGSGTDFTLTISSLQPEDFATYYC | |
| | | QQYDSFPYTFGQGTKVEIK | |
| | VL CDR1 | QDISPY | SEQ ID NO: 619 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-27 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDISYNLNWYQHKPGKA | SEQ ID NO: 698 |
| | | PKPLVFGSPNLETGVPSRFSASGSGTDFTLTISSLQPEDFATYYC | |
| | | QQYDSFPYTFGQGTKVEIK | |
| | VL CDR1 | QDISYN | SEQ ID NO: 622 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-28 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIEWWLNWYQHKPGK | SEQ ID NO: 699 |
| | | APKPLIYGSPNLETGVPSRFSGSGSGTDFTFTISSLQPEDIATYYC | |
| | | QQYDSFPYTFGQGTKVEIK | |
| | VL CDR1 | QDIEWW | SEQ ID NO: 615 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLIY | SEQ ID NO: 636 |
| | VL FR3 | NLETGVPSRFSGSGSGTDFTFTISSLQPEDIATYYC | SEQ ID NO: 639 |
| | VL FR4 | FGQGTKVEIK | SEQ ID NO: 642 |
| L1-29 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPWLNWYQHKPGK | SEQ ID NO: 700 |
| | | APKPLVFDAPNLETGVPSRFSASGSGTDFTLTISSLQPEDFATYY | |
| | | CQQYDSFPYTFGQGTKVEIK | |
| | VL CDR1 | QDIGPW | SEQ ID NO: 614 |
| | VL CDR2 | DAP | SEQ ID NO: 629 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-30 | VH | QVQLVESGGGVVQPGRSLRLSCAASGFTFSTYGMHWVRQAP | SEQ ID NO: 670 |
| | | GKGLEWVAVIWDDGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDDGSYK | SEQ ID NO: 602 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPWLNWYQHKPGK | SEQ ID NO: 701 |
| | | APKPLVFGADNLETGVPSRFSASGSGTDFTLTISSLQPEDFATY | |
| | | YCQQYDSFPYTFGQGTKVEIK | |
| | VL CDR1 | QDIGPW | SEQ ID NO: 614 |
| | VL CDR2 | GAD | SEQ ID NO: 628 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-31 | VH | QVQLVESGGGVVQPGRSLRLSCAASGFTFSTYGMHWVRQAP | SEQ ID NO: 671 |
| | | GKGLEWVAVIWDGGKYKYYGDSVKGRFTISRDNSKNTLYLQ | |
| | | MNSLRAEDTAVYYCARDGSGWGYAFDIWGQGTLVTVSS | |
| | VH CDR1 | GFTFSTYG | SEQ ID NO: 601 |
| | VH CDR2 | IWDGGKYK | SEQ ID NO: 603 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPWLNWYQHKPGK | SEQ ID NO: 702 |
| | | APKPLVFRSPNLETGVPSRFSASGSGTDFTLTISSLQPEDFATYY | |
| | | CQQYDSFPYTFGQGTKVEIK | |
| | VL CDR1 | QDIGPW | SEQ ID NO: 614 |
| | VL CDR2 | RSP | SEQ ID NO: 624 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-32 | VH | QVQLVESGGGVVQPGRSLRLSCAASGFTFSTYGMHWVRQAP | SEQ ID NO: 672 |
| | | GKGLEWVAVIWDGGSYKYYGDSVKGRFTISRDNSKNTLYLQM | |
| | | NSLRAEDTAVYYCARDGSGWGYAFDIWGQGTLVTVSS | |
| | VH CDR1 | GFTFSTYG | SEQ ID NO: 601 |
| | VH CDR2 | IWDGGSYK | SEQ ID NO: 604 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPWLNWYQHKPGK | SEQ ID NO: 694 |
| | | APKPLVFGSPNLETGVPSRFSASGSGTDFTLTISSLQPEDFATYY | |
| | | CQQYDSFPYTFGQGTKVEIK | |
| | VL CDR1 | QDIGPW | SEQ ID NO: 614 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |
| L1-33 | VH | QVQLVESGGGVVQPGRSLRLSCAASGFTFSTYGMHWVRQAP | SEQ ID NO: 671 |
| | | GKGLEWVAVIWDGGKYKYYGDSVKGRFTISRDNSKNTLYLQ | |
| | | MNSLRAEDTAVYYCARDGSGWGYAFDIWGQGTLVTVSS | |
| | VH CDR1 | GFTFSTYG | SEQ ID NO: 601 |
| | VH CDR2 | IWDGGKYK | SEQ ID NO: 603 |
| | VH CDR3 | ARDGSGWGYAFDI | SEQ ID NO: 605 |
| | VH FR1 | QVQLVESGGGVVQPGRSLRLSCAAS | SEQ ID NO: 606 |
| | VH FR2 | MHWVRQAPGKGLEWVAV | SEQ ID NO: 607 |
| | VH FR3 | YYGDSVKGRFTISRDNSKNTLYLQMNSLRAEDTAVYYC | SEQ ID NO: 608 |
| | VH FR4 | WGQGTLVTVSS | SEQ ID NO: 610 |
| | VL | DIQLTQSPSSLSASVGDRVTITCQASQDIGPWLNWYQHKPGK | SEQ ID NO: 694 |
| | | APKPLVFGSPNLETGVPSRFSASGSGTDFTLTISSLQPEDFATYY | |
| | | CQQYDSFPYTFGQGTKVEIK | |
| | VL CDR1 | QDIGPW | SEQ ID NO: 614 |
| | VL CDR2 | GSP | SEQ ID NO: 625 |
| | VL CDR3 | QQYDSFPYT | SEQ ID NO: 631 |
| | VL FR1 | DIQLTQSPSSLSASVGDRVTITCQAS | SEQ ID NO: 632 |
| | VL FR2 | LNWYQHKPGKAPKPLVF | SEQ ID NO: 637 |
| | VL FR3 | NLETGVPSRFSASGSGTDFTLTISSLQPEDFATYYC | SEQ ID NO: 640 |
| | VL FR4 | FGQGTKVEIK | SEQ ID NO: 642 |

TABLE 3
Representative mutations on heavy or light
chain of L2 that decreased affinity

| | ka (1/Ms) | kd (1/s) | KD (M) |
|---|---|---|---|
| L2-01 | 3.38E+04 | 2.82E−04 | 8.34E−09 |
| L2VH-R30S | 1.14E+04 | 1.65E−03 | 1.46E−07 |
| L2VH-D31S | 1.22E+04 | 5.67E−03 | 4.65E−07 |
| L2VH-S50A | 2.05E+04 | 4.86E−04 | 2.37E−08 |
| L2VH-T35S | 6.11E+04 | 1.39E−03 | 2.28E−08 |
| L2VH-R98K | 5.10E+04 | 2.48E−03 | 4.87E−08 |
| L2VL-L49Y | 3.08E+02 | 4.71E−03 | 1.53E−05 |
| L2VL-I53S | 1.35E+03 | 1.73E−03 | 1.28E−06 |
| L2VL-G56S | 1.07E+05 | 3.11E−03 | 2.91E−08 |
| L2VL-S52V | 1.25E+04 | 8.06E−04 | 6.45E−08 |
| L2VL-D93S | 2.97E+04 | 2.02E−03 | 6.80E−08 |

TABLE 4
Antibodies derived from L2 with increased affinity

| | ka (1/Ms) | kd (1/s) | K_{D} (M) |
|---|---|---|---|
| L2-01 | 3.38E+04 | 2.82E−04 | 8.34E−09 |
| L2-02 | 2.71E+04 | 1.31E−04 | 4.85E−09 |
| L2-03 | 1.42E+05 | 4.65E−04 | 3.27E−09 |
| L2-04 | 4.76E+04 | 1.42E−04 | 2.98E−09 |
| L2-05 | 4.27E+04 | 1.12E−04 | 2.63E−09 |
| L2-06 | 4.57E+04 | 2.49E−04 | 5.45E−09 |
| L2-07 | 1.11E+05 | 1.29E−04 | 1.16E−09 |
| L2-08 | 1.29E+05 | 1.12E−04 | 8.69E−10 |
| L2-09 | 1.51E+05 | 7.15E−05 | 4.75E−10 |
| L2-10 | 1.44E+05 | 3.68E−05 | 2.56E−10 |

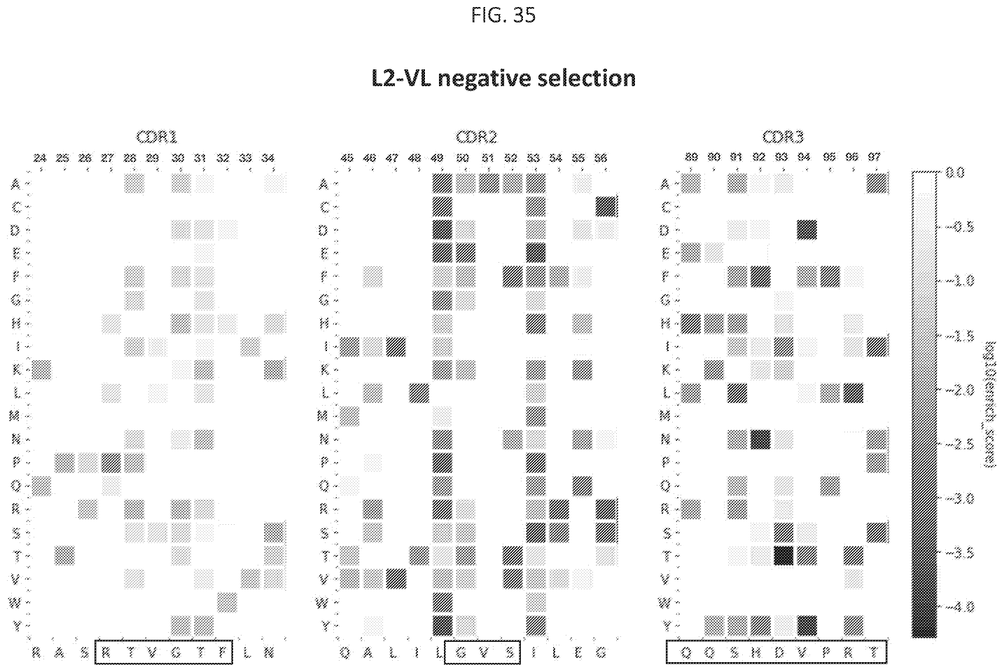

TABLE E
Permissible amino acid substitutions in CDRs of the L2 VH
that do not negatively affect binding of L2 (see FIG. 33)

| | | |
|---|---|---|
| | Original | |
| Position | amino acid | Permissible amino acids |

CDR1

| | | |
|---|---|---|
| 26 | G | A, C, D, E, F, G, H, I, K, L, M, N, P, Q, R, S, T, V, W or Y |
| 27 | F | A, C, D, E, F, G, H, K, L, M, N, P, Q, R, S, T, V or W, e.g., G |
| 28 | T | A, C, D, E, F, G, H, I, K, L, M, N, P, Q, R, T, V, W or Y |
| 29 | F | A, C, D, E, F, G, H, I, K, M, N, P, Q, R, S, T, V, W or Y |
| 30 | R | I, K, L, M, P, Q, V, W or Y, e.g., K |
| 31 | D | F, G, H, I, K, L, M, P, Q, T, V, W or Y |
| 32 | Y | A, C, D, E, F, G, H, I, K, M, N, P, Q, R, S, T, V, W or Y, e.g., F or N, |
| | | more preferably F |
| 33 | A | A, C, D, E, F, H, K, L, M, N, P, Q, R, T or V |
| 34 | M | A, C, D, E, F, G, H, K, M, N, P, Q, R, S, T, V, W or Y |
| 35 | T | A, C, D, E, F, I, K, L, M, P, Q, R, T, V, W or Y |

CDR2

| | | |
|---|---|---|
| 50 | S | A, C, D, E, F, H, K, L, N, P, Q, R, S or Y, e.g., A |
| 51 | I | A, C, D, E, G, H, I, K, L, M, N, P, Q, R, S, T, V, W or Y |
| 52 | S | A, C, E, F, G, H, I, K, L, M, P, Q, R, S, V or Y |
| 53 | G | C, D, E, F, G, H, I, K, L, M, N, Q, R, S or V |
| 54 | S | A, C, F, H, K, L, M, P, Q, R, S, T, V or W, e.g., M |
| 55 | G | A, C, E, F, G, H, I, K, L, M, P, Q, R, V or W |
| 56 | G | A, C, D, E, F, G, H, I, K, L, M, N, P, Q, R, T, V, W or Y |
| 57 | N | A, C, F, G, I, K, L, M, N, P, Q, R, S, T, V, W or Y, e.g., G, N, R, S, or T, |
| | | more preferably G or S |
| 58 | T | C, D, E, F, G, H, K, L, M, N, P, Q, S, T, V, W or Y |
| 59 | Y | A, C, E, H, K, L, M, P, S, T, V, W or Y, e.g., K |
| 60 | Y | A, C, D, E, F, G, H, I, K, L, M, N, P, Q, R, T, V, W or Y |
| 61 | A | A, C, D, E, F, H, I, K, L, M, N, P, Q, R, S, T, V, W or Y |
| 62 | D | A, C, D, E, F, G, H, I, K, L, M, N, R, S, T, V, W or Y |
| 63 | S | A, C, D, F, G, H, I, L, M, N, P, Q, R, S, T, V, W or Y, e.g., N |
| 64 | V | A, C, D, E, G, H, I, K, L, M, N, P, Q, R, S, T, V, W or Y |
| 65 | K | A, C, D, F, G, H, I, K, L, M, N, P, S, T, V, W or Y |
| 66 | G | A, C, E, F, G, H, I, K, L, M, N, P, Q, R, S, T, V, W or Y |

CDR3

| | | |
|---|---|---|
| 97 | A | A, C, D, E, F, G, H, I, K, L, M, N, P, Q, R, S, T, V, W or Y |
| 98 | R | F, G, H, I or T |
| 99 | G | A, C, D, F, G, H, I, K, L, M, N, P, Q, R, S, T, V, W or Y |
| 100 | E | D, E, F, H, N, Q, R, S, T, V, W or Y, e.g., D |
| 101 | Y | A, H, I, L, M, N, Q or Y |
| 102 | S | A, C, D, F, G, H, K, M, N, P, Q, R, S, T, V or Y |
| 103 | S | A, C, E, F, H, K, M, N, P, Q, S, T, W or Y |
| 104 | G | A, C, D, E, F, G, H, I, K, L, M, N, P, Q, S, T, V or W |
| 105 | W | A, C, D, E, F, H, I, K, L, N, Q, R, S, V, W or Y |
| 106 | P | A, C, D, E, F, H, I, K, L, M, N, P, Q, R, S, V, W or Y |
| 107 | S | A, C, E, F, H, I, K, L, M, N, P, Q, S, T, V, W or Y |
| 108 | W | F, H, I, K, N, P, Q, R, V, W or Y, e.g., F or Y |
| 109 | F | A, C, D, E, F, G, H, I, K, L, N, P, Q, R, S, T, V, W or Y |
| 110 | D | D, F, G, H, P, Q or T, e.g., T |
| 111 | P | D, E, F, G, I, K, L, N, P, Q, R, S or T |

In the table above, residues after “e.g.” are residue changes that enhance binding compared to the original residue.

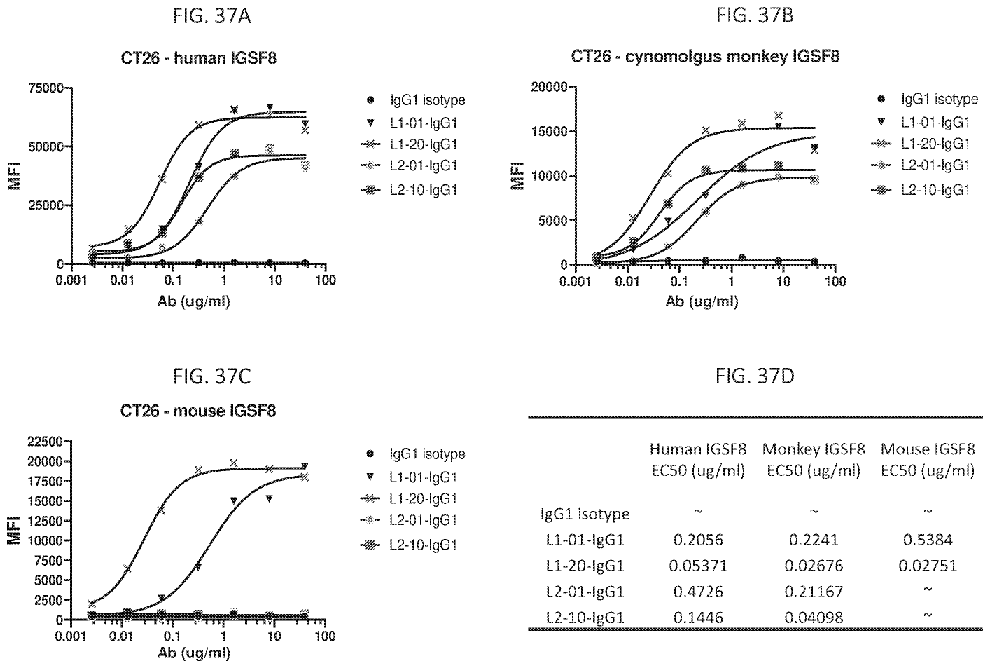

TABLE F
Permissible amino acid substitutions in CDRs of the L2 VL
that do not negatively affect binding of L2 (see FIG. 35)

| | | |
|---|---|---|
| | Original | |
| Position | amino acid | Permissible amino acids |

CDR1

| | | |
|---|---|---|
| 24 | R | A, C, D, E, F, G, H, I, L, M, N, P, R, S, T, V, W or Y |
| 25 | A | A, C, D, E, F, G, H, I, K, L, M, N, Q, R, S, V, W or Y |
| 26 | S | A, C, D, E, F, G, H, I, K, L, M, N, Q, S, T, V, W or Y |
| 27 | R | A, C, D, E, F, G, I, K, M, N, R, S, T, V, W or Y, e.g., E |
| 28 | T | C, D, E, H, K, L, M, Q, T, W or Y, e.g., D |
| 29 | V | A, C, D, E, F, G, H, K, M, N, P, Q, R, T, V, W or Y |
| 30 | G | C, E, G, I, L, M, P, Q, V, W or Y |
| 31 | T | C, M, P, Q, T or W, e.g., P |
| 32 | F | A, C, E, F, G, I, K, L, M, N, P, Q, R, T, V or Y, e.g., Y |
| 33 | L | A, C, D, E, F, G, H, K, L, M, N, P, Q, R, S, T, W or Y |
| 34 | N | C, D, E, F, G, I, L, M, N, P, Q, R, W or Y, e.g., G |

CDR2

| | | |
|---|---|---|
| 45 | Q | A, C, D, E, F, G, H, K, L, N, P, R, S, W or Y, e.g., K, N, or R, more |
| | | preferably K |
| 46 | A | A, C, D, E, G, H, K, M, N, Q, T or W |
| 47 | L | A, C, D, E, F, G, H, K, L, M, N, P, Q, R, S, T, W or Y |
| 48 | I | A, C, D, E, F, G, H, I, K, M, N, P, Q, R, S, V, W or Y, e.g., V |
| 49 | L | L |
| 50 | G | C, H, I, L, M, N, P, Q, W or Y, e.g., H or Q |
| 51 | V | C, D, E, F, G, H, I, K, L, M, N, P, Q, R, S, T, V, W or Y, e.g., S, T, or V, |
| | | more preferably S or T |
| 52 | S | C, D, E, G, H, I, K, L, M, P, Q, R, S, W or Y |
| 53 | I | I |
| 54 | L | A, C, D, E, G, H, I, K, L, M, N, P, Q, T, W or Y |
| 55 | E | C, E, G, I, L, M, P, R, S, T, W or Y, e.g., R |
| 56 | G | A, E, F, G, H, I, K, L, M, P, Q, V, W or Y, e.g., A, I, L, or P |

CDR3

| | | |
|---|---|---|
| 89 | Q | C, D, F, G, I, K, M, N, P, Q, S, T, V, W or Y |
| 90 | Q | A, C, D, F, G, I, L, M, N, P, Q, R, S, T, V or W |
| 91 | S | C, E, G, K, M, P, S, V or W |
| 92 | H | C, H, L, M, P, Q, R, V or W |
| 93 | D | C, D, E, F, L, M, P, V or W, e.g., F |
| 9 | V | A, C, E, G, H, K, M, N, P, Q, R, V or W, e.g., A, N, P, R, or W |
| 95 | P | A, C, D, E, G, H, I, K, M, N, P, R, S, T, V, W or Y |
| 96 | R | A, C, D, E, G, K, M, N, P, Q, R, S or W, e.g., D, S, or W |
| 97 | T | C, D, E, F, G, H, K, L, M, Q, R, T, V, W or Y |

In the table above, residues after “e.g.” are residue changes that enhance binding compared to the original residue.

+0 TABLE G
Sequences of selected antibodies derived from L2 with increased affinity

| Antibody | | Sequence | SEQ ID NO: |
|---|---|---|---|
| L2-01 | VH | EVQLVESGGGLEQPGGSLRLSCAGSGFTFRDYAMTWVRQAP | SEQ ID NO: 703 |
| | | GKGLEWVSSISGSGGNTYYADSVKGRFTISRDNSKNTLYLQM | |
| | | NSLRAEDTAIYYCARGEYSSGWPSWFDPWGQGTLVTVSS | |
| | VH CDR1 | GFTFRDYA | SEQ ID NO: 643 |
| | VH CDR2 | ISGSGGNT | SEQ ID NO: 644 |
| | VH CDR3 | ARGEYSSGWPSWFDP | SEQ ID NO: 646 |
| | VH FR1 | EVQLVESGGGLEQPGGSLRLSCAGS | SEQ ID NO: 647 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | EIVLTQSPSSLSASVGDRVTISCRASRTVGTFLNWYQQRPGKPP | SEQ ID NO: 707 |
| | | QALILGVSILEGGVSSRFSGAGSGTEFSLTISSLQPEDIGTYYCQ | |
| | | QSHDVPRTFGQGTKVDLR | |
| | VL CDR1 | RTVGTF | SEQ ID NO: 652 |
| | VL CDR2 | GVS | SEQ ID NO: 653 |
| | VL CDR3 | QQSHDVPRT | SEQ ID NO: 655 |
| | VL FR1 | EIVLTQSPSSLSASVGDRVTISCRAS | SEQ ID NO: 656 |
| | VL FR2 | LNWYQQRPGKPPQALIL | SEQ ID NO: 658 |
| | VL FR3 | ILEGGVSSRFSGAGSGTEFSLTISSLQPEDIGTYYC | SEQ ID NO: 660 |
| | VL FR4 | FGQGTKVDLR | SEQ ID NO: 664 |
| L2-02 | VH | EVQLVESGGGLEQPGGSLRLSCAGSGFTFRDYAMTWVRQAP | SEQ ID NO: 703 |
| | | GKGLEWVSSISGSGGNTYYADSVKGRFTISRDNSKNTLYLQM | |
| | | NSLRAEDTAIYYCARGEYSSGWPSWFDPWGQGTLVTVSS | |
| | VH CDR1 | GFTFRDYA | SEQ ID NO: 643 |
| | VH CDR2 | ISGSGGNT | SEQ ID NO: 644 |
| | VH CDR3 | ARGEYSSGWPSWFDP | SEQ ID NO: 646 |
| | VH FR1 | EVQLVESGGGLEQPGGSLRLSCAGS | SEQ ID NO: 647 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | EIVLTQSPSSLSASVGDRVTISCRASRTVGTFLNWYQQRPGKPP | SEQ ID NO: 708 |
| | | QALILHTSILEGGVSSRFSGAGSGTEFSLTISSLQPEDIGTYYCQQ | |
| | | SHDVPRTFGQGTKVDLR | |
| | VL CDR1 | RTVGTF | SEQ ID NO: 652 |
| | VL CDR2 | HTS | SEQ ID NO: 654 |
| | VL CDR3 | QQSHDVPRT | SEQ ID NO: 655 |
| | VL FR1 | EIVLTQSPSSLSASVGDRVTISCRAS | SEQ ID NO: 656 |
| | VL FR2 | LNWYQQRPGKPPQALIL | SEQ ID NO: 658 |
| | VL FR3 | ILEGGVSSRFSGAGSGTEFSLTISSLQPEDIGTYYC | SEQ ID NO: 660 |
| | VL FR4 | FGQGTKVDLR | SEQ ID NO: 664 |
| L2-03 | VH | EVQLVESGGGLEQPGGSLRLSCAGSGFTFRDYAMTWVRQAP | SEQ ID NO: 704 |
| | | GKGLEWVSSISGSGGSTYYADSVKGRFTISRDNSKNTLYLQMN | |
| | | SLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAGS | SEQ ID NO: 647 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | EIVLTQSPSSLSASVGDRVTISCRASRTVGTFLNWYQQRPGKPP | SEQ ID NO: 707 |
| | | QALILGVSILEGGVSSRFSGAGSGTEFSLTISSLQPEDIGTYYCQ | |
| | | QSHDVPRTFGQGTKVDLR | |
| | VL CDR1 | RTVGTF | SEQ ID NO: 652 |
| | VL CDR2 | GVS | SEQ ID NO: 653 |
| | VL CDR3 | QQSHDVPRT | SEQ ID NO: 655 |
| | VL FR1 | EIVLTQSPSSLSASVGDRVTISCRAS | SEQ ID NO: 656 |
| | VL FR2 | LNWYQQRPGKPPQALIL | SEQ ID NO: 658 |
| | VL FR3 | ILEGGVSSRFSGAGSGTEFSLTISSLQPEDIGTYYC | SEQ ID NO: 660 |
| | VL FR4 | FGQGTKVDLR | SEQ ID NO: 664 |
| L2-04 | VH | EVQLVESGGGLEQPGGSLRLSCAASGFTFRDYAMTWVRQAP | SEQ ID NO: 705 |
| | | GKGLEWVSSISGSGGSTYYADSVKGRFTISRDNSKNTLYLQMN | |
| | | SLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAAS | SEQ ID NO: 648 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | EIVLTQSPSSLSASVGDRVTISCRASRTVGTFLNWYQQRPGKPP | SEQ ID NO: 707 |
| | | QALILGVSILEGGVSSRFSGAGSGTEFSLTISSLQPEDIGTYYCQ | |
| | | QSHDVPRTFGQGTKVDLR | |
| | VL CDR1 | RTVGTF | SEQ ID NO: 652 |
| | VL CDR2 | GVS | SEQ ID NO: 653 |
| | VL CDR3 | QQSHDVPRT | SEQ ID NO: 655 |
| | VL FR1 | EIVLTQSPSSLSASVGDRVTISCRAS | SEQ ID NO: 656 |
| | VL FR2 | LNWYQQRPGKPPQALIL | SEQ ID NO: 658 |
| | VL FR3 | ILEGGVSSRFSGAGSGTEFSLTISSLQPEDIGTYYC | SEQ ID NO: 660 |
| | VL FR4 | FGQGTKVDLR | SEQ ID NO: 664 |
| L2-05 | VH | EVQLVESGGGLEQPGGSLRLSCAASGFTFRDYAMTWVRQAP | SEQ ID NO: 706 |
| | | GKGLEWVSAISGSGGSTYYADSVKGRFTISRDNSKNTLYLQM | |
| | | NSLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAAS | SEQ ID NO: 648 |
| | VH FR2 | MTWVRQAPGKGLEWVSA | SEQ ID NO: 650 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | EIVLTQSPSSLSASVGDRVTISCRASRTVGTFLNWYQQRPGKPP | SEQ ID NO: 707 |
| | | QALILGVSILEGGVSSRFSGAGSGTEFSLTISSLQPEDIGTYYCQ | |
| | | QSHDVPRTFGQGTKVDLR | |
| | VL CDR1 | RTVGTF | SEQ ID NO: 652 |
| | VL CDR2 | GVS | SEQ ID NO: 653 |
| | VL CDR3 | QQSHDVPRT | SEQ ID NO: 655 |
| | VL FR1 | EIVLTQSPSSLSASVGDRVTISCRAS | SEQ ID NO: 656 |
| | VL FR2 | LNWYQQRPGKPPQALIL | SEQ ID NO: 658 |
| | VL FR3 | ILEGGVSSRFSGAGSGTEFSLTISSLQPEDIGTYYC | SEQ ID NO: 660 |
| | VL FR4 | FGQGTKVDLR | SEQ ID NO: 664 |
| L2-06 | VH | EVQLVESGGGLEQPGGSLRLSCAASGFTFRDYAMTWVRQAP | SEQ ID NO: 705 |
| | | GKGLEWVSSISGSGGSTYYADSVKGRFTISRDNSKNTLYLQMN | |
| | | SLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAAS | SEQ ID NO: 648 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | DIQMTQSPSSLSASVGDRVTITCRASRTVGTFLNWYQQKPGK | SEQ ID NO: 709 |
| | | APKALILGVSILEGGVPSRFSGSGSGTDFTLTISSLQLEDFATYYC | |
| | | QQSHDVPRTFGQGTKVEIK | |
| | VL CDR1 | RTVGTF | SEQ ID NO: 652 |
| | VL CDR2 | GVS | SEQ ID NO: 653 |
| | VL CDR3 | QQSHDVPRT | SEQ ID NO: 655 |
| | VL FR1 | DIQMTQSPSSLSASVGDRVTITCRAS | SEQ ID NO: 657 |
| | VL FR2 | LNWYQQKPGKAPKALIL | SEQ ID NO: 659 |
| | VL FR3 | ILEGGVPSRFSGSGSGTDFTLTISSLQLEDFATYYC | SEQ ID NO: 661 |
| | VL FR4 | FGQGTKVEIK | SEQ ID NO: 665 |
| L2-07 | VH | EVQLVESGGGLEQPGGSLRLSCAASGFTFRDYAMTWVRQAP | SEQ ID NO: 705 |
| | | GKGLEWVSSISGSGGSTYYADSVKGRFTISRDNSKNTLYLQMN | |
| | | SLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAAS | SEQ ID NO: 648 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | DIQMTQSPSSLSASVGDRVTITCRASRTVGTFLNWYQQKPGK | SEQ ID NO: 710 |
| | | APKALILGVSILEGGVPSRFSGSGSGTDFTLTISSLQPEDIGTYYC | |
| | | QQSHDVPRTFGQGTKVEIK | |
| | VL CDR1 | RTVGTF | SEQ ID NO: 652 |
| | VL CDR2 | GVS | SEQ ID NO: 653 |
| | VL CDR3 | QQSHDVPRT | SEQ ID NO: 655 |
| | VL FR1 | DIQMTQSPSSLSASVGDRVTITCRAS | SEQ ID NO: 657 |
| | VL FR2 | LNWYQQKPGKAPKALIL | SEQ ID NO: 659 |
| | VL FR3 | ILEGGVPSRFSGSGSGTDFTLTISSLQPEDIGTYYC | SEQ ID NO: 662 |
| | VL FR4 | FGQGTKVEIK | SEQ ID NO: 665 |
| L2-08 | VH | EVQLVESGGGLEQPGGSLRLSCAASGFTFRDYAMTWVRQAP | SEQ ID NO: 705 |
| | | GKGLEWVSSISGSGGSTYYADSVKGRFTISRDNSKNTLYLQMN | |
| | | SLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAAS | SEQ ID NO: 648 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | DIQMTQSPSSLSASVGDRVTITCRASRTVGTFLNWYQQKPGK | SEQ ID NO: 711 |
| | | APKALILGVSILEGGVPSRFSGSGSGTDFTLTISSLQPEDIATYYC | |
| | | QQSHDVPRTFGQGTKVEIK | |
| | VL CDR1 | RTVGTF | SEQ ID NO: 652 |
| | VL CDR2 | GVS | SEQ ID NO: 653 |
| | VL CDR3 | QQSHDVPRT | SEQ ID NO: 655 |
| | VL FR1 | DIQMTQSPSSLSASVGDRVTITCRAS | SEQ ID NO: 657 |
| | VL FR2 | LNWYQQKPGKAPKALIL | SEQ ID NO: 659 |
| | VL FR3 | ILEGGVPSRFSGSGSGTDFTLTISSLQPEDIATYYC | SEQ ID NO: 663 |
| | VL FR4 | FGQGTKVEIK | SEQ ID NO: 665 |
| L2-09 | VH | EVQLVESGGGLEQPGGSLRLSCAASGFTFRDYAMTWVRQAP | SEQ ID NO: 705 |
| | | GKGLEWVSSISGSGGSTYYADSVKGRFTISRDNSKNTLYLQMN | |
| | | SLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAAS | SEQ ID NO: 648 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | DIQMTQSPSSLSASVGDRVTITCRASRTVGTFLNWYQQKPGK | SEQ ID NO: 712 |
| | | APKALILHTSILEGGVPSRFSGSGSGTDFTLTISSLQPEDIGTYYC | |
| | | QQSHDVPRTFGQGTKVEIK | |
| | VL CDR1 | RTVGTF | SEQ ID NO: 652 |
| | VL CDR2 | HTS | SEQ ID NO: 654 |
| | VL CDR3 | QQSHDVPRT | SEQ ID NO: 655 |
| | VL FR1 | DIQMTQSPSSLSASVGDRVTITCRAS | SEQ ID NO: 657 |
| | VL FR2 | LNWYQQKPGKAPKALIL | SEQ ID NO: 659 |
| | VL FR3 | ILEGGVPSRFSGSGSGTDFTLTISSLQPEDIGTYYC | SEQ ID NO: 662 |
| | VL FR4 | FGQGTKVEIK | SEQ ID NO: 665 |
| L2-10 | VH | EVQLVESGGGLEQPGGSLRLSCAASGFTFRDYAMTWVRQAP | SEQ ID NO: 705 |
| | | GKGLEWVSSISGSGGSTYYADSVKGRFTISRDNSKNTLYLQMN | |
| | | SLRAEDTAIYYCARGEYSSGWPSWFDPWGQGTLVTVSS | |
| | VH CDR1 | GFTFRDYA | SEQ ID NO: 643 |
| | VH CDR2 | ISGSGGST | SEQ ID NO: 645 |
| | VH CDR3 | ARGEYSSGWPSWFDP | SEQ ID NO: 646 |
| | VH FR1 | EVQLVESGGGLEQPGGSLRLSCAAS | SEQ ID NO: 648 |
| | VH FR2 | MTWVRQAPGKGLEWVSS | SEQ ID NO: 649 |
| | VH FR3 | YYADSVKGRFTISRDNSKNTLYLQMNSLRAEDTAIYYC | SEQ ID NO: 651 |
| | VH FR4 | WGQGTLVTVSS | SEQ ID NO: 610 |
| | VL | DIQMTQSPSSLSASVGDRVTITCRASRTVGTFLNWYQQKPGK | SEQ ID NO: 713 |
| | | APKALILHTSILEGGVPSRFSGSGSGTDFTLTISSLQPEDIATYYC | |
| | | QQSHDVPRTFGQGTKVEIK | |
| | VL CDR1 | RTVGTF | SEQ ID NO: 652 |
| | VL CDR2 | HTS | SEQ ID NO: 654 |
| | VL CDR3 | QQSHDVPRT | SEQ ID NO: 655 |
| | VL FR1 | DIQMTQSPSSLSASVGDRVTITCRAS | SEQ ID NO: 657 |
| | VL FR2 | LNWYQQKPGKAPKALIL | SEQ ID NO: 659 |
| | VL FR3 | ILEGGVPSRFSGSGSGTDFTLTISSLQPEDIATYYC | SEQ ID NO: 663 |
| | VL FR4 | FGQGTKVEIK | SEQ ID NO: 665 |

### Example 26 Characterization of Monoclonal Antibody Binding to IGSF8 Expressed on the Surface of Cells

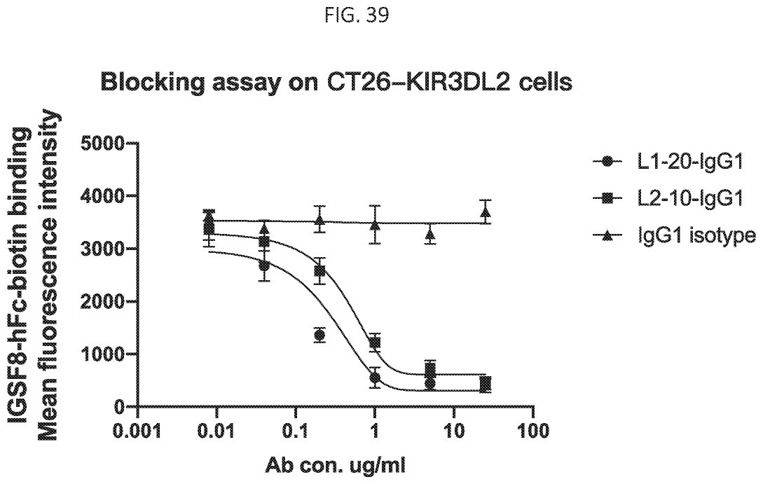

To test cross reactivity of the L1, and L2 antibodies, CT26 cells with forced human, cynomolgus monkey, or mouse IGSF8 expression were generated by lentiviral delivery. For antibody binding assays based on cell-expressed antigens, 100,000 cells overexpressing the antigen were washed with PBS buffer containing 2% FBS, and then incubated with 100 μL of antibodies or IgG of different concentrations for 30 minutes on ice. Cells were then washed twice with wash buffer, and incubated with 100 μL of 1:100 anti-human Fc-PE antibody for 15 minutes on ice. Cells were washed twice with wash buffer and analyzed on a FACS analyzer (CytoFlex, Beckman). The MFI was plotted vs. antibody concentration. The EC50 cell binding potenct was calculated using nonlinear regression curve fit. FIG. 37 shows that the L1 antibody had cross reactivity with the IGSF8 antigen from all of these tested species, while the L2 antibody can cross-react with human and monkey IGSF8, but not mouse antibody. Compare to the original sequences of the L1 and L2 antibodies, the optimized versions (L1-20 and L1-10) were randomly selected, and were found to have markedly increased EC50 cell binding potency (Table 5).

TABLE 5
EC50 of binding of L1 and L2 antibodies to IGSF8

| | Human IGSF8 | Monkey IGSF8 | Mouse IGSF8 |
|---|---|---|---|
| | EC50 (ug/ml) | EC50 (ug/ml) | EC50 (ug/ml) |
| IgG1 isotype | ~ | ~ | ~ |
| L1-01-IgG1 | 0.2056 | 0.2241 | 0.5384 |
| L1-20-IgG1 | 0.05371 | 0.02676 | 0.02751 |
| L2-01-IgG1 | 0.4726 | 0.21167 | ~ |
| L2-10-IgG1 | 0.1446 | 0.04098 | ~ |

### Example 27 IGSF8 Suppression of Primary NK Cell Cytotoxicity is Mediated by KIR3DL2

KIR3DL2 was found to be the receptor of IGSF8 on NK or T cells. This example demonstrates that IGSF8 acts as a suppression ligand expressed on cancer cells, and regulates the cytotoxicity of NK or T cells by binding to KIR3DL2.

For this experiment, an NK cell-K562 co-culture model was used, in which the MHC-I deficient K562 cell line—a known NK-sensitive cell line—was chosen as the target cell line for NK cell-mediated killing. Specifically, primary NK cells were seeded in 96-well plates (10,000 cells per well) and co-cultured with K562 cells at E/T ratio=5:1 for 2 hours. The dead cells were stained by 7-AAD (Biolegend) before the cells were analyzed by flow cytometry. NK cell cytotoxicity was calculated by NK cytotoxicity (%)=(7-AAD+%(dead) K562 cells cocultured with NK cells)−(spontaneously 7-AAD+%(dead) K562 cells without coculture with NK cells).

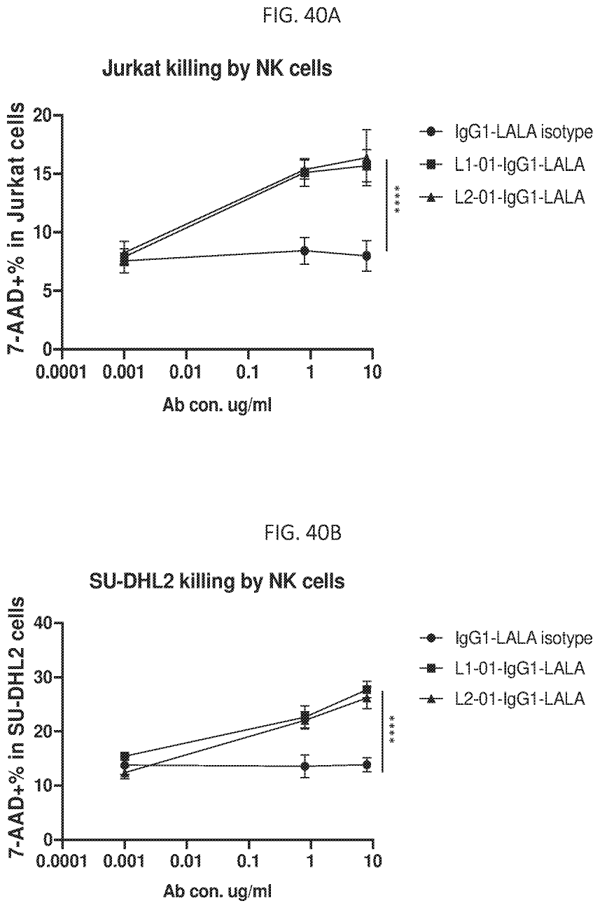

First, forced expression of IGSF8 protein on K562 cells by lentiviral delivery of an IGSF8-expression construct mostly protected the IGSF8-expressing K562 cells from primary NK cell killing (FIG. 38B).

Second, loss of KIR3DL2 expression by lentiviral-mediated CRISPR/Cas9 knock down of any endogenous KIR3DL2 expression in primary NK cells remarkably increased the susceptibility both of the K562 and K562-IGSF8 cells. There is no difference between the killing of K562 and K562-IGSF8 cells (see FIGS. 38A-38B).

These data suggests that the cytotoxicity of the NK cells with KIR3DL2 knockout can not be suppressed by IGSF8, indicating IGSF8 is a functional ligand of KIR3DL2 for mediating the NK cytotoxicity.

### Example 28 Antibodies Block the Interactions Between IGSF8 and KIR3DL2

This example demonstrates that the subject anti-IGSF8 antibodies are capable of blocking the interaction between IGSF8 and its ligand KIR3DL2, based on a flow cytometry-based assay.

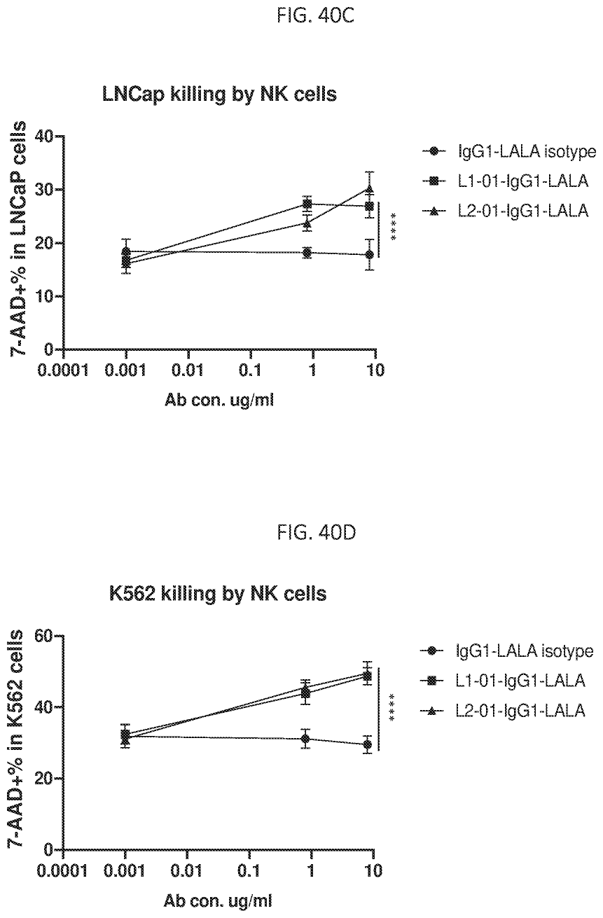

CT26 cells with forced ectopic/exogenous human KIR3DL2 expression were incubated with biotinylated IGSF8-hFc recombinant protein (1 μg/mL) in the presence of the antibodies L1-20, L2-10, and a isotype control antibody. The binding of IGSF8-hFc-biotin to the Ct26 cells was determined using Streptavidin-PE by flow cytometry analysis (FIG. 39). The results showed that both L1-20 and L2-10 can completely block the interactions between IGSF8 and KIR3DL2 in a dose dependent manner, while the control had no effect.

### Example 29 the Anti-IGSF8 mAbs Enhances NK or PBMC Killing of Different Cancer Cells

As IGSF8 has been found to express on many different cancer cell types/cell lines, in vitro NK or PBMC killing assays were designed to show that IGSF8 blockade by the antibodies of the invention can enhance NK or PBMC cytotoxicity to different cancer cells.

Carboxyfluorescein succinimidyl ester (CFSE, ThermoFisher Scientific) labeling of cancer cells combined with 7-AAD (Biolegend) staining of dead cells is a reliable method to measure in vitro cell killing by flow cytometry. Briefly, NK cells or PBMCs were cocultured with CFSE-labeled Cancer cells with E/T ratio=1:1 or 5:1 in U-bottom 96-well plates for 4 hours. Then, each sample was mixed with 7-AAD and analyzed by FACS. Cell lysis was calculated by the percentage of 7-AAD-positive cancer cells among total cancer cells. Spontaneous cancer cell death, in the absence of NK or PBMCs, was less than 5%, and subtracted from total killing in the presence of NK or PBMCs.

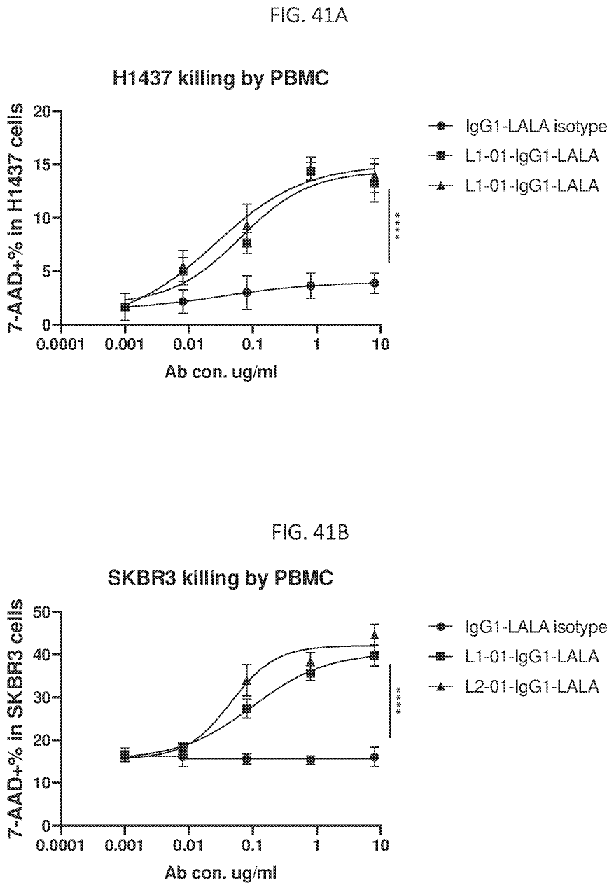

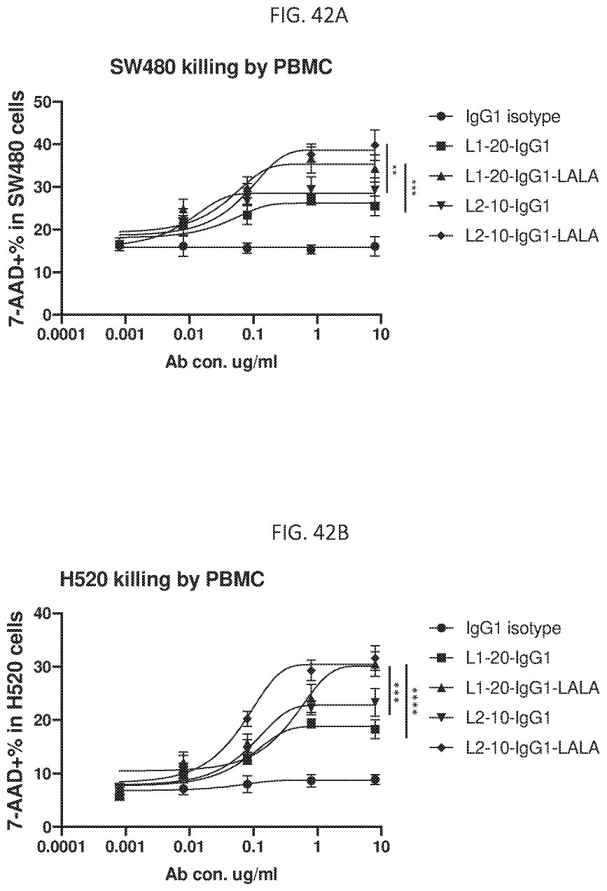

To exclude the confounding factor of IgG1-Fc mediated Antibody-Dependent Cellular Cytotoxicity (ADCC) in this experiment, a L234A and L235A mutated human IgG1 (IgG1-LALA), which has previously been shown to have largely lost the ADCC effect of human IgG1, was used in this assay. As shown in FIGS. 40A-40D, 41A-41B and 42A-42B, treatment with the L1-20 and L2-10 mAbs remarkably sensitized the NK or PBMC cytotoxicity to numerous cancer cell lines, including Jurkat (T cell leukemia), SU-DHL2 (large cell lymphoma), LNCaP (prostate carcinoma), K562 (chronic myelogenous leukemia), H1437 (lung adenocarcinoma), SKBR3 (breast adenocarcinoma), SW480 (colorectal adenocarcinoma), and H520 (lung squamous cell carcinoma).

As the anti-IGSF8 monoclonal antibodies with deficient IgG1-mediated ADCC can still enhance NK or PBMC cytotoxicity to cancer cells, it was expected that the subject anti-IGSF8 monoclonal antibodies with wildtype constant region sequence of IgG1, which has normal effector functions (such as complement-dependent cutotoxicity (CDC), antibody-dependent cell-mediated cytotoxicity (ADCC), and antibody-dependent cellular phagocytosis (ADCP)), would be able to maintain or even further improve PBMC cytotoxicity.

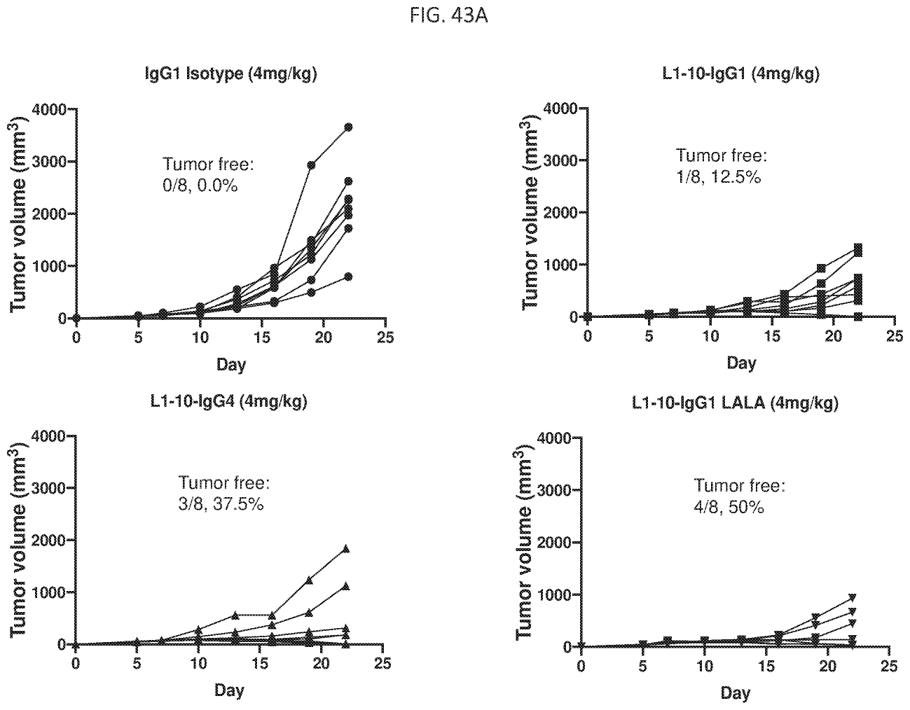

Unexpectedly, it was found that, compared to anti-IGSF8 monoclonal antibodies with deficient IgG1 effector function (such as IgG1-LALA), anti-IGSF8 monoclonal antibodies with normal effector functions had significantly decreased PBMC cytotoxicity to two types of cancer cell lines, SW480 and H520 (FIGS. 42A-42B). These data suggest that cancer treatment using the subject anti-IGSF8 antibodies not only is independent of the Fc effector functions of these antibodies, but also benefits from the presence of a deficient Fc.

### Example 30 Anti-IGSF8 mAb with Deficient IgG1 Showed Better In Vivo Anti-Tumor Efficacy than the mAb with Normal IgG1 or IgG4

As L1 and L2 antibodies with deficient Fc functions were shown to better enhance NK or PBMC killing of different cancer cells (Example 29), in vivo efficacy of these antibodies was further examined by the B16-F10 syngeneic mouse model, which was known to be resistant to current immunotherapy (anti-PD1/L1, anti-CTLA4 et, al.).

In particular, about one million B16-F10 melanoma cells were inoculated subcutaneously into wild type (WT) C57BL/6 mice. four groups of mice (n=8 mice per group) were then treated with the L1-10 antibody with human IgG1, IgG4 and IgG1-LALA, as well as one IgG1 isotype antibody, respectively, at a dose of about 4 mg/kg, from Day 7 post tumor cell innoculation, every 3 days, for a total of four doses, by tail vein injection. Data were presented as mean±s.e.m.

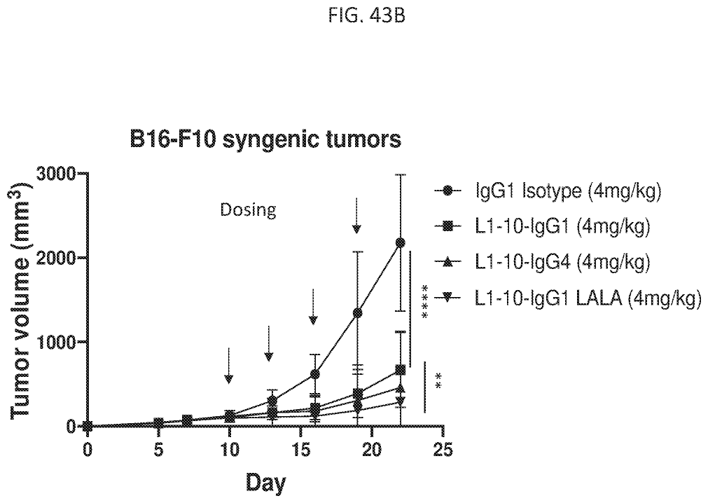

As shown in FIG. 43A, treatment of the B16 tumors with L1-10-IgG1 significantly and dramatically reduced tumor growth compared to isotype-matched control. However, the L1-10-IgG4 which was known to have weak ADCC but strong ADCP effector functions had better efficacy to reduce tumor growth. The L1-10-IgG4 group had 37.5% (3/8) tumor free mice than the L1-10-IgG1 group. More strikingly, the L1-10-IgG1-LALA with depleted ADCC or ADCP effector functions had the best anti-tumor efficacy in vivo (FIG. 43B). Four of the eight (50%) mice treated with L1-10-IgG1-LALA achieved complete response (CR), with no detectable tumor, after the treatment (FIG. 43A). These data suggest that the treating cancers using the anti-IGSF8 antibodies of the invention benefits from the presence of a deficient Fc with depleted ADCC or ADCP effector functions.

### Example 31 the Anti-IGSF8 mAbs with Effector Fc Functions Impaired Effector NK and T Cells Infiltration into the B16 Tumors

To identify the mechanism by which treatment of B16-F10 tumors using the IGSF8 mAbs with deficient Fc functions had better efficacy than the IGSF8 mAbs with normal IgG1 and IgG4 (Example 30), when the in vivo study was completed, the B16 tumors treated by different IGSF8 mAbs (three samples per arm), were isolated, and RNA-sequencing was performed on isolated tumors. The TPM (Transcript per million) score was calculated by DESeq2 (Genome Biology, 15, 550. doi: 10.1186/s13059-014-0550-8.)

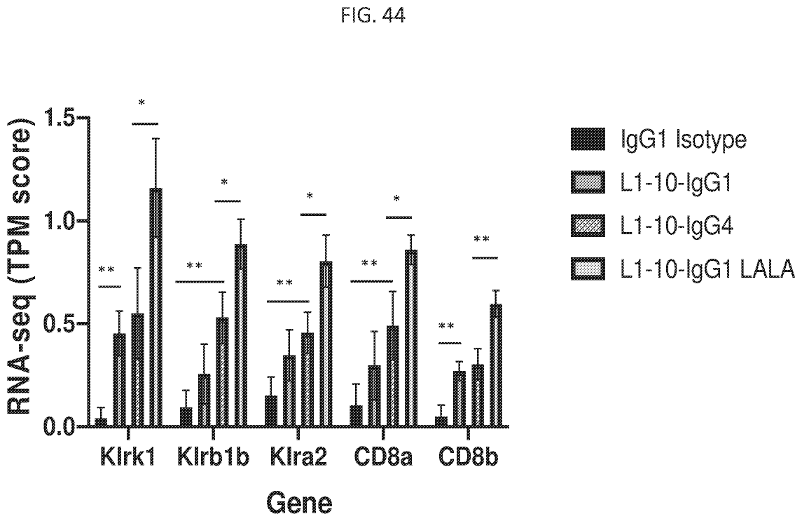

Based on the RNA-seq data, it was found that the gene markers (Klrk1, Klrb1b and Klra2) representing the effector NK cells and the gene markers (CD8a and CD8b) representing the effector T cells were significantly up-regulated in the tumors treated by different IGSF8 mAbs compared to the control groups (FIG. 44). These suggest the anti-IGSF8 treatments strongly reprogramed the Tumor Microenvironment (TME) to increase the NK and T cells infiltrations. Moreover, these marker genes are markedly increased in the groups treated by the IGSF8 mAb with deficient Fc compared to the groups treated by the mAbs with IgG1 and IgG4, indicating that the anti-IGSF8 mAbs with normal effector Fc functions impaired effector NK and T cells infiltration into the B16 tumors.

These data could explain the reason that treating cancers through the anti-IGSF8 antibodies benefits from the deficient Fc with depleted ADCC, CDC and ADCP effector functions.

We claim:

1. An isolated or recombinant monoclonal antibody or an antigen-binding fragment thereof specific for IGSF8 (e.g., specific for the Ig-V set domain or the D1 domain of the ECD of IGSF8), wherein said monoclonal antibody or antigen-binding fragment thereof comprises a heavy chain variable region (VH) comprising a VH CDR1, a VH CDR2, and a VH CDR3, and a light chain variable region (VL) comprising a VL CDR1, a VL CDR2 and a VL CDR3, wherein
(a1) the VH CDR1, VH CDR2 and VH CDR3 comprise, consists essentially of, or consists of the amino acid sequence of SEQ ID NOs: 714, 715 and 716, respectively; and the VL CDR1, VL CDR2 and VL CDR3 comprise, consists essentially of, or consists of the amino acid sequence of SEQ ID NOs: 717, 718 and 719, respectively; or
(a2) the VH CDR1, VH CDR2 and VH CDR3 comprise, consists essentially of, or consists of the amino acid sequence of SEQ ID NOs: 754, 755 and 756, respectively; and the VL CDR1, VL CDR2 and VL CDR3 comprise, consists essentially of, or consists of the amino acid sequence of SEQ ID NOs: 757, 758 and 759, respectively; or
(b1) the VH CDR1, VH CDR2 and VH CDR3 comprise, consists essentially of, or consists of the amino acid sequence of SEQ ID NOs: 720, 721 and 722, respectively; and the VL CDR1, VL CDR2 and VL CDR3 comprise, consists essentially of, or consists of the amino acid sequence of SEQ ID NOs: 723, 724 and 725, respectively; or
(b2) the VH CDR1, VH CDR2 and VH CDR3 comprise, consists essentially of, or consists of the amino acid sequence of SEQ ID NOs: 760, 761 and 762, respectively; and the VL CDR1, VL CDR2 and VL CDR3 comprise, consists essentially of, or consists of the amino acid sequence of SEQ ID NOs: 763, 764 and 765, respectively; or
(c) the VH CDR1, VH CDR2 and VH CDR3 comprise, consists essentially of, or consists of the amino acid sequence of any of the VH CDR1, VH CDR2 and VH CDR3 sequences, respectively, of Table D and Table G; and the VL CDR1, VL CDR2 and VL CDR3 comprise, consists essentially of, or consists of the amino acid sequence of any of the VL CDR1, VL CDR2 and VL CDR3 sequences, respectively, of Table D and Table G; or
(d) the VH CDR1, VH CDR2, VH CDR3, VL CDR1, VL CDR2, and VL CDR3 comprise, consists essentially of, or consists of the amino acid sequence of the VH CDR1, VH CDR2 VH CDR3, VL CDR1, VL CDR2, and VL CDR3 sequences, respectively, of any one antibody of Table D and Table G;
optionally, the antibody and the antigen-binding fragment thereof do not have the same VH CDR1, VH CDR2 VH CDR3, VL CDR1, VL CDR2, and VL CDR3 sequences of the L1 antibody and that of the L2 antibody (e.g., the the antibody is not L1 and is not L2).

2. The monoclonal antibody or an antigen-binding fragment thereof of claim 1, wherein:
(1) the VH CDR1, VH CDR2, VH CDR3, VL CDR1, VL CDR2, and VL CDR3 comprise the amino acid sequences of the VH CDR1, VH CDR2 VH CDR3, VL CDR1, VL CDR2, and VL CDR3 sequences, respectively, of any one antibody of Table D; or
(2) the VH CDR1, VH CDR2, VH CDR3, VL CDR1, VL CDR2, and VL CDR3 comprise the amino acid sequences of the VH CDR1, VH CDR2 VH CDR3, VL CDR1, VL CDR2, and VL CDR3 sequences, respectively, of any one antibody of Table G.

3. The monoclonal antibody or an antigen-binding fragment thereof of claim 1 or 2, wherein
(a) the VH comprises a VH FR1, a VH FR2, a VH FR3, and/or a VH FR4 comprising (i) the amino acid sequence(s) of the corresponding VH FR sequence(s) of any one or more antibodies in Table D (or Table G), (ii) an amino acid sequence having at least 90%, 91%, 92%, 93%, 94%, 95%, 96%, 97%, 98%, 99% sequence identity to the corresponding VH FR sequence(s) of any one or more antibodies in Table D (or Table G); or (iii) an amino acid sequence having at most 1, 2, 3, 4, or 5 substitutions, deletions, and/or additions compared to the corresponding VH FR sequence(s) of any one or more antibodies in Table D (or Table G); and/or
(b) the VL comprises a VL FR1, a VL FR2, a VL FR3, and/or a VL FR4 comprising (i) the amino acid sequence(s) of the corresponding VL FR sequence(s) of any one or more antibodies in Table D (or Table G), (ii) an amino acid sequence having at least 90%, 91%, 92%, 93%, 94%, 95%, 96%, 97%, 98%, 99% sequence identity to the corresponding VL FR sequence(s) of any one or more antibodies in Table D (or Table G); or (iii) an amino acid sequence having at most 1, 2, 3, 4, or 5 substitutions, deletions, and/or additions compared to the corresponding VL FR sequence(s) of any one or more antibodies in Table D (or Table G).

4. The monoclonal antibody or an antigen-binding fragment thereof of claim 1 or 2, wherein
(a1) the VH comprises the amino acid sequence of SEQ ID NOs: 734, 735 and 736, respectively; and the VL comprises the amino acid sequence of SEQ ID NOs: 737, 738 and 739, respectively; or
(a2) the VH comprises the amino acid sequence of SEQ ID NOs: 774, 775 and 776, respectively; and the VL comprises the amino acid sequence of SEQ ID NOs: 777, 778 and 779, respectively; or
(b1) the VH comprises the amino acid sequence of SEQ ID NOs: 740, 741 and 742, respectively; and the VL comprises the amino acid sequence of SEQ ID NOs: 743, 744 and 745, respectively; or
(b2) the VH comprises the amino acid sequence of SEQ ID NOs: 780, 781 and 782, respectively; and the VL comprises the amino acid sequence of SEQ ID NOs: 783, 784 and 785, respectively; or
(c) the VH comprises the amino acid sequence of any VH sequence of Table D and Table G; and the VL comprises the amino acid sequence of any VL sequence of Table D and Table G.

5. The monoclonal antibody or an antigen-binding fragment thereof of any one of claims 1-4, wherein the VH and VL seqeuences comprise the amino acid sequences of the VH and VL sequences, respectively, of any one antibody of Table D and Table G.

6. The monoclonal antibody or antigen-binding fragment thereof of any one of claims 1-5, which is a human-mouse chimeric antibody, a humanized antibody, a human antibody, a CDR-grafted antibody, or a resurfaced antibody.

7. The monoclonal antibody or antigen-binding fragment thereof of any one of claims 1-6, wherein said antigen-binding fragment thereof is an Fab, Fab′, F(ab′)₂, F_{d}, single chain Fv or scFv, disulfide linked F, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

8. The monoclonal antibody or antigen-binding fragment thereof of any one of claims 1-7, comprising a heavy chain constant region, wherein
(a) the heavy chain constant region is wild-type human IgG1, human IgG2, human IgG3, human IgG4; or
(b) the heavy chain constant region has an Fc domain deficient in antibody-dependent cellular cytotoxicity (ADCC), complement-dependent cytotoxicity (CDC) and/or antibody-dependent cellular phagocytosis (ADCP).

9. The monoclonal antibody or antigen-binding fragment thereof of claim 8, wherein the heavy chain constant region with an deficient Fc domain is selected from a group consisting of IgG1-L234A/L235A (IgG1-LALA), IgG1-L234A/L235A/P329G (IgG1-LALA-PG), IgG1-N297A/Q/G (IgG1-NA), IgG1-L235A/G237A/E318A (IgG1-AAA), IgG1-G236R/L328R (IgG1-RR), IgG1-S298G/T299A (IgG1-GA), IgG1-L234F/L235E/P331S (IgG1-FES), IgG1-L234F/L235E/D265A (IgG1-FEA), IgG4-L234A/L235A (IgG4-LALA), IgG4-S228P/L235E (IgG4-PE), IgG1-E233P/L234V/L235A/G236del/S267K, IgG2-H268Q/V309L/A30S/P331S (IgG2m4) and IgG2-V234A/G237A/P238S/H268A/V309L/A330S/P331S (IgG2c4d).

10. The monoclonal antibody or antigen-binding fragment thereof of any one of claims 1-9, wherein said monoclonal antibody or antigen-binding fragment thereof binds IGSF8 with a K_{d} of less than about 25 nM, 20 nM, 15 nM, 10 nM, 5 nM, 2 nM, or 1 nM.

11. A monoclonal antibody or an antigen-binding fragment thereof, which competes with the monoclonal antibody or antigen-binding fragment thereof of any one of claims 1-10 for binding to IGSF8.

12. The antibody or monoclonal antibody of any one of claims 1-11, wherein the antibody or antigen-binding portion/fragment thereof inhibits IGSF8 binding to KIR3DL1/2.

13. A polynucleotide encoding a monoclonal antibody of any one of claims 1-12, a heavy chain or a light chain thereof, or an antigen-binding portion/fragment thereof.

14. A polynucleotide that hybridizes under stringent conditions with the polynucleotide of claim 13, or with a complement of the polynucleotide of claim 13.

15. A vector comprising the polynucleotide of claim 13 or 14.

16. A host cell comprising the polynucleotide of claim 13 or 14, or the vector of claim 15, for expressing the encoded monoclonal antibody, heavy or light chain thereof, or antigen-binding portion/fragment thereof.

17. A method of producing the monoclonal antibody, heavy or light chain thereof, or antigen-binding portion/fragment thereof of any one of claims 1-12, the method comprising:
(i) culturing the host cell of claim 15 capable of expressing said monoclonal antibody, heavy or light chain thereof, or antigen-binding portion/fragment thereof under a condition suitable to express said monoclonal antibody, heavy or light chain thereof, or antigen-binding portion/fragment thereof; and, optionally
(ii) recovering/isolating/purifying the expressed monoclonal antibody, heavy or light chain thereof, or antigen-binding portion/fragment thereof.

18. A method of modulating an immune response in a subject in need thereof, the method comprising administrating a therapeutically effective amount of the anti-IGSF8 monoclonal antibody or antigen-binding fragment thereof of any one of claims 1-12 to the subject.

19. A method of treating a cancer in a subject in need thereof, the method comprising administrating a therapeutically effective amount of the anti-IGSF8 monoclonal antibody or antigen-binding fragment thereof of any one of claims 1-12 to the subject.

20. The method of claim 18 or 19, further comprising administering to the subject an effective amount of a second therapeutic agent comprising an immunotherapy, an immune checkpoint inhibitor, a cancer vaccine, a chimeric antigen receptor, a chemotherapeutic agent, a radiation therapy, an anti-angiogenesis agent, a growth inhibitory agent, an immune-oncology agent, an anti-neoplastic composition, a surgery, or a combination thereof.

21. The method of any one of claims 18-20, wherein the anti-IGSF8 monoclonal antibody or antigen-binding fragment thereof is conjugated to a cytotoxic agent.

22. The method of claim 21, wherein the cytotoxic agent is selected from the group consisting of a chemotherapeutic agent, a biologic agent, a toxin, and a radioactive isotope.

23. The method of any one of claims 19-21, wherein the anti-IGSF8 monoclonal antibody or antigen-binding fragment thereof reduces the number of proliferating cells in the cancer and/or reduces the volume or size of a tumor of the cancer.

24. The method of any one of claims 18-23, wherein the anti-IGSF8 monoclonal antibody or antigen-binding fragment thereof is administered in a pharmaceutically acceptable formulation.

25. The method of any one of claims 19-24, wherein the cancer is melanoma (including skin cutaneous melanoma), cervical cancer, lung cancer (e.g., non-small cell lung cancer, lung adenocarcinoma, lung squamous cell carcinoma), colorectal cancer, lymphoma (including B cell lymphoma and DLBCL), leukemia (including CLL and Acute Myeloid Leukemia (AML)), BLCA tumor, breast cancer, head and neck carcinoma, head-neck squamous cell carcinoma, PRAD, THCA, or UCEC, thyroid cancer, unitary tract cancer, uterine cancer, esophagus cancer, liver cancer, ganglia cancer, renal cancer, pancreatic cancer, pancreatic ductal carcinoma, ovarian cancer, prostate cancer, gliomas, glioblastoma, neuroblastoma, thymoma, B-CLL, and a cancer infiltrated with immune cells expressing a receptor to IGSF8.

26. The method of any one of claims 19-25, wherein the cancer is lung cancer, renal cancer, pancreatic cancer, colorectal cancer, acute myeloid leukemia (AML), head and neck carcinoma, liver cancer, ovarian cancer, prostate cancer, or uterine cancer.

27. The method of any one of claims 19-26, wherein the cancer cells and/or tumor immune infiltrating cells in the subject express IGSF8.

28. The method of any one of claims 19-27, wherein the anti-IGSF8 monoclonal antibody or antigen-binding fragment thereof stimulates T cell and/or NK cell activation and/or infiltration into tumor microenvironment.

29. The method of any one of claims 20-28, wherein the immune checkpoint inhibitor is an antibody or antigen-binding fragment thereof specific for PD-1, PD-L1, PD-L2, LAG3, TIGIT, TIM3, NKG2A, CD276, VTCN1, VISR or HHLA2.

30. The method of claim 29, wherein the immune checkpoint inhibitor is an anti-PD-1 antibody, such as cemiplimab, nivolumab, or pembrolizumab.

31. The method of claim 29, wherein the immune checkpoint inhibitor is an anti-PD-L1 antibody, such as avelumab, durvalumab, atezolizumab, KN035, or CK-301.

32. The method of any one of claims 20-28, wherein the immune checkpoint inhibitor is a (non-antibody) peptide inhibitor of PD-1/PD-L1, such as AUNP12; a small molecule inhibitor of PD-L1 such as CA-170, or a macrocyclic peptide such as BMS-986189.

33. The method of any one of claims 20-32, wherein said second therapeutic agent comprises an antibody or an antigen-binding portion/fragment thereof effective to treat a cancer, such as 3F8, 8H9, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Amivantamab, Anatumomab mafenatox, Andecaliximab, Anetumab ravtansine, Anifrolumab, Anrukinzumab, Apolizumab, Aprutumab ixadotin, Arcitumomab, Ascrinvacumab, Aselizumab, Atezolizumab, Atidortoxumab, Atinumab, Atorolimumab, Avelumab, Azintuxizumab vedotin, Bapineuzumab, Basiliximab, Bavituximab, BCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, BirtamimabBivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab, Carlumab, Carotuximab, Catumaxomab, cBR-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, GancotamabGanitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ianalumab, Ibalizumab, IBI308, Ibritumomab tiuxetan, Icrucumab, Idarucizumab, Ifabotuzumab, Igovomab, Iladatuzumab vedotin, IMAB363, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Mapatumumab, Margetuximab, MarstacimabMaslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, OtilimabOtlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, RanevetmabRanibizumab, Raxibacumab, Ravagalimab, Ravulizumab, Refanezumab, Regavirumab, REGN-EB, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Talquetamab, Tamtuvetmab, Tanezumab, Taplitumomab paptox, Tarextumab, TavolimabTeclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, tiragolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab, Zolbetuximab, (=IMAB362, Claudiximab), Zolimomab aritox, or combination thereof.

34. The method of any one of claims 20-33 wherein said second therapeutic agent comprises an antibody or an antigen-binding portion/fragment thereof is effective to induce ADCC, ADCP and/or CDC.

35. The method of any one of claims 19-34, wherein the subject is an animal model of a cancer.

36. A device or kit comprising at least one antibody, monoclonal antibody, heavy or light chain thereof, or antigen-binding portion/fragment thereof, according to any one of claims 1-12, said device or kit optionally comprising a label to detect said at least one antibody, monoclonal antibody, heavy or light chain thereof, or antigen-binding portion/fragment thereof, or a complex comprising said at least one antibody, monoclonal antibody, heavy or light chain thereof, or antigen-binding portion/fragment thereof.

37. A method of detecting the presence or level of an IGSF8 polypeptide in a sample, the method comprising contacting the IGSF8 polypeptide in the sample with the antibody, monoclonal antibody, or antigen-binding portion/fragment thereof, according to any one of claims 1-12, wherein said antibody, monoclonal antibody, or antigen-binding portion/fragment thereof is labeled by a detectable label, or can be attached to a detectable label.

38. The method of claim 37, wherein said antibody, monoclonal antibody, or antigen binding portion/fragment thereof, forms a complex with the IGSF8 polypeptide, and the complex is detected in the form of an enzyme linked immunosorbent assay (ELISA), radioimmune assay (RIA), immunochemical method, Western blot, or an intracellular flow assay.

39. A method for monitoring the progression of a disorder associated with aberrant (e.g., higher than normal) IGSF8 expression in a subject, the method comprising:
a) detecting, in a sample obtained from the subject, at a first point in time a first level of IGSF8 using the antibody, monoclonal antibody, or antigen-binding portion/fragment thereof, according to any one of claims 1-12;
b) repeating step a) at a subsequent point in time to obtain a second level of IGSF8; and
c) comparing the first and the second levels of IGSF8 detected in steps a) and b), respectively, to monitor the progression of the disorder in the subject,
wherein a higher second level than the first level is indicative that the disease has progressed.

40. The method of claim 39, wherein between the first point in time and the subsequent point in time, the subject has undergone a treatment to ameliorate the disorder.

41. A method for predicting the clinical outcome of a subject afflicted with a disorder associated with aberrant (e.g., higher than normal) IGSF8 expression, the method comprising:
a) determining the level of IGSF8 in a first sample obtained from the subject, using the antibody, monoclonal antibody, or antigen-binding portion/fragment thereof, according to any one of claims 1-12;
b) determining the level of IGSF8 in a second sample obtained from a control subject having a good clinical outcome, using the antibody, monoclonal antibody, or antigen-binding portion/fragment thereof, according to any one of claims 1-12; and
c) comparing the level of IGSF8 in the first and the second samples;
wherein a significantly higher (e.g., >20%, >50% or more increase) level of IGSF8 in the first sample as compared to the level of IGSF8 in the second sample is an indication that the subject has a worse clinical outcome, and/or,
wherein a significantly lower (e.g., >20%, >50% or more decrease) level of IGSF8 in the first sample as compared to the level of IGSF8 in the second sample is an indication that the subject has a better clinical outcome.

42. A method of assessing the efficacy of a therapy for a disorder associated with aberrant (e.g., higher than normal) IGSF8 expression in a subject, the method comprising:
a) determining the level of IGSF8 using the antibody, monoclonal antibody, or antigen-binding portion/fragment thereof, according to any one of claims 1-12, in a first sample obtained from the subject prior to providing at least a portion of the therapy to the subject, and
b) repeat step a) in a second sample obtained from the subject following provision of said portion of the therapy,
wherein a significantly lower (>20%, >50% or more decrease) level of IGSF8 in the second sample, relative to the first sample, is an indication that the therapy is efficacious for inhibiting the disorder in the subject; and/or,
wherein a substantially identical or higher level of IGSF8 in the second sample, relative to the first sample, is an indication that the therapy is not efficacious for inhibiting the disorder in the subject.

43. The method of claim 41 or 42, wherein the disease is cancer.

44. A method of assessing the efficacy of a test compound for inhibiting a disorder associated with aberrant (e.g., higher than normal) IGSF8 expression in a subject, the method comprising:
a) determining the level of IGSF8 using the antibody, monoclonal antibody, or antigen-binding portion/fragment thereof, according to any one of claims 1-12, in a first sample obtained from the subject, wherein the first sample has been exposed to an amount of the test compound; and
b) determining the level of IGSF8 using the antibody, monoclonal antibody, or antigen-binding portion/fragment thereof, according to any one of claims 1-12, in a second sample obtained from the subject, wherein the second sample has not been exposed to the test compound,
wherein a significantly lower (>20%, >50% or more decrease) level of IGSF8 in the first sample relative to that of the second sample, is an indication that the amount of the test compound is efficacious for inhibiting the disorder in the subject, and/or,
wherein a substantially identical level of IGSF8 in the first sample relative to that of the second sample, is an indication that the amount of the test compound is not efficacious for inhibiting the disorder in the subject.

45. The method of claim 44, wherein the first and second samples are portions of a single sample obtained from the subject or portions of pooled samples obtained from the subject.

46. The method of claim 44 or 45, wherein the disorder is a cancer.

47. The method of claim 46, wherein the cancer is lung cancer, renal cancer, pancreatic cancer, colorectal cancer, Acute myeloid leukemia (AML), head and neck carcinoma, liver cancer, ovarian cancer, prostate cancer, uterine cancer, gliomas, glioblastoma, neuroblastoma, breast cancer, pancreatic ductal carcinoma, thymoma, B-CLL, leukemia, B cell lymphoma, and a cancer infiltrated with immune cells (e.g., T cells and/or NK cells) expressing a receptor to IGSF8 (e.g., KIR3DL1, KIR3DL2, and/or KLRC1/D1).

48. The method of any one of claims 37-47, wherein the sample comprises cells, serum, peritumoral tissue, and/or intratumoral tissue obtained from the subject.

49. The method of any one of claims 39-48, wherein the subject is a human.

50. A monoclonal antibody or an antigen-binding fragment thereof specific for IGSF8, wherein said monoclonal antibody comprises:
(1) a heavy chain variable region (HCVR), comprising HCVR CDR1-CDR3 sequences at least 95% (e.g., 100%) identical to, or having up to 1, 2, 3, 4, 5, 6, 7, 8, or 9 substitutions in HCVR CDR1-CDR3, respectively, of any one of antibodies C1-C39, such as C30-C39; and,
(2) a light chain variable region (LCVR), comprising LCVR CDR1-CDR3 sequences at least 95% (e.g., 100%) identical to, or having up to 1, 2, 3, 4, 5, 6, 7, 8, or 9 substitutions in LCVR CDR1-CDR3, respectively, of any one of antibodies C1-C39, such as C30-C39.

51. The monoclonal antibody or antigen-binding fragment thereof of claim 50, comprising:
(a) HCVR sequence at least 95% (e.g., 100%) identical to the HCVR sequence of any one of antibodies C1-C39, such as C30-C39; and/or,
(b) LCVR sequence at least 95% (e.g., 100%) identical to the LCVR sequence of any one of antibodies C1-C39, such as C30-C39.

52. The monoclonal antibody or antigen-binding fragment thereof of claim 50 or 51, which is a human-mouse chimeric antibody, a humanized antibody, a human antibody, a CDR-grafted antibody, or a resurfaced antibody.

53. The monoclonal antibody or antigen-binding fragment thereof of any one of claims 50-52, wherein said antigen-binding fragment thereof is an Fab, Fab′, F(ab′)₂, F_{d}, single chain Fv or scFv, disulfide linked F, V-NAR domain, IgNar, intrabody, IgGΔCH₂, minibody, F(ab′)3, tetrabody, triabody, diabody, single-domain antibody, DVD-Ig, Fcab, mAb₂, (scFv)₂, or scFv-Fc.

54. The monoclonal antibody or antigen-binding fragment thereof of any one of claims 50-53, wherein said monoclonal antibody or antigen-binding fragment thereof binds IGSF8 with a K_{d} of less than about 25 nM, 20 nM, 15 nM, 10 nM, 5 nM, 2 nM, or 1 nM.

55. A monoclonal antibody or an antigen-binding fragment thereof, which competes with the monoclonal antibody or antigen-binding fragment thereof of any one of claims 50-54 for binding to IGSF8.

56. The antibody or monoclonal antibody of any one of claims 50-55, wherein the antibody or antigen-binding portion/fragment thereof specifically binds the D1 ECD (or Ig-V set domain) of IGSF8, preferably with a K_{D} of no more than 5 nM, 2 nM, or 1 nM.

57. The antibody or monoclonal antibody of any one of claims 50-56, wherein the antibody or antigen-binding portion/fragment thereof inhibits IGSF8 binding to KIR3DL1/2.

58. The antibody or monoclonal antibody of any one of claims 50-57, wherein the antibody or antigen-binding portion/fragment thereof inhibits IGSF8 binding to the D2 domain of KIR3DL1/2, such as an epitope comprising S165, I171, and/or M186 of KIR3DL1/2.

59. A monoclonal antibody or an antigen-binding portion/fragment thereof, which specifically binds the D1 ECD (or Ig-V set domain) of IGSF8, and inhibits binding to KIR3DL1/2, such as binding to the D2 domain of KIR3DL1/2 (e.g., an epitope comprising S165, I171, and/or M186 of KIR3DL1/2).

60. The monoclonal antibody or antigen-binding portion/fragment thereof of claim 59, having a K_{D} of no more than 5 nM, 2 nM, or 1 nM.

61. A polynucleotide encoding a monoclonal antibody of any one of claims 50-60, a heavy chain or a light chain thereof, or an antigen-binding portion/fragment thereof.

62. A polynucleotide that hybridizes under stringent conditions with the polynucleotide of claim 61, or with a complement of the polynucleotide of claim 61.

63. A vector comprising the polynucleotide of claim 61 or 62.

64. A host cell comprising the polynucleotide of claim 61 or 62, or the vector of claim 63, for expressing the encoded monoclonal antibody, heavy or light chain thereof, or antigen-binding portion/fragment thereof.

65. A method of producing the monoclonal antibody, heavy or light chain thereof, or antigen-binding portion/fragment thereof of any one of claims 50-60, the method comprising:
(i) culturing the host cell of claim 64 capable of expressing said monoclonal antibody, heavy or light chain thereof, or antigen-binding portion/fragment thereof under a condition suitable to express said monoclonal antibody, heavy or light chain thereof, or antigen-binding portion/fragment thereof, and
(ii) recovering/isolating/purifying the expressed monoclonal antibody, heavy or light chain thereof, or antigen-binding portion/fragment thereof.

66. A method of modulating an immune response in a subject in need thereof, the method comprising inhibiting interaction between IGSF8 and a receptor of IGSF8 selected from KIR3DL1, KIR3DL2, and KLRC1/D2 heterodimer.

67. A method of immunotherapy for treating a cancer in a subject in need thereof, the method comprising inhibiting interaction between IGSF8 and a receptor of IGSF8 selected from KIR3DL1, KIR3DL2, and KLRC1/D2 heterodimer.

68. A method of treating cancer in a subject in need thereof, the method comprising administering to the subject a therapeutically effective amount of an IGSF8 (Immuno Globulin Super Family 8) modulator (e.g., antagonist).

69. A method of treating cancer in a subject in need thereof, the method comprising administering to the subject a therapeutically effective amount of an KIR3DL1 antagonist that inhibits interaction with IGSF8.

70. A method of treating cancer in a subject in need thereof, the method comprising administering to the subject a therapeutically effective amount of an KIR3DL2 antagonist that inhibits interaction with IGSF8.

71. A method of treating cancer in a subject in need thereof, the method comprising administering to the subject a therapeutically effective amount of an KLRC1/D1 antagonist that inhibits interaction with IGSF8.

72. The method of any one of claims 66-71, further comprising administering to the subject an effective amount of a second therapeutic agent comprising an immunotherapy, an immune checkpoint inhibitor, a cancer vaccine, a chimeric antigen receptor, a chemotherapeutic agent, a radiation therapy, an anti-angiogenesis agent, a growth inhibitory agent, an immune-oncology agent, an anti-neoplastic composition, a surgery, or a combination thereof.

73. The method of any one of claims 66-68 and 72, comprising administering to the subject an IGSF8 antagonist selected from an anti-IGSF8 antibody or an antigen-binding portion/fragment thereof, an inhibitory peptide of IGSF8, a nucleic acid targeting IGSF8 (an aptamer, an antisense polynucleotide, an RNAi reagent such as siRNA, miRNA, shRNA; a guide RNA for a Type 2 CRISPR/Cas effector enzyme), or a small molecule targeting IGSF8 (e.g., with M.W.<1000 Da or <500 Da); optionally, the IGSF8 antagonist is the anti-IGSF8 antibody or antigen-binding portion/fragment thereof.

74. The method of any one of claims 66, 67, 69, and 72, comprising administering to the subject a KIR3DL1 antagonist selected from an anti-KIR3DL1 antibody or an antigen-binding portion/fragment thereof, an inhibitory peptide of KIR3DL1, a nucleic acid targeting KIR3DL1 (an aptamer, an antisense polynucleotide, an RNAi reagent such as siRNA, miRNA, shRNA; a guide RNA for a Type 2 CRISPR/Cas effector enzyme), or a small molecule targeting KIR3DL1 (e.g., with M.W.<1000 Da or <500 Da); optionally, the KIR3DL1 antagonist is the anti-KIR3DL1 antibody or antigen-binding portion/fragment thereof.

75. The method of any one of claims 66, 67, 70, and 72, comprising administering to the subject a KIR3DL2 antagonist selected from an anti-KIR3DL2 antibody or an antigen-binding portion/fragment thereof, an inhibitory peptide of KIR3DL2, a nucleic acid targeting KIR3DL2 (an aptamer, an antisense polynucleotide, an RNAi reagent such as siRNA, miRNA, shRNA; a guide RNA for a Type 2 CRISPR/Cas effector enzyme), or a small molecule targeting KIR3DL2 (e.g., with M.W.<1000 Da or <500 Da); optionally, the KIR3DL2 antagonist is the anti-KIR3DL2 antibody or antigen-binding portion/fragment thereof.

76. The method of any one of claims 66, 67, 71, and 72, comprising administering to the subject a KLRC1/D1 antagonist selected from an anti-KLRC1/D1 antibody or an antigen-binding portion/fragment thereof, an inhibitory peptide of KLRC1/D1, a nucleic acid targeting KLRC1/D1 (an aptamer, an antisense polynucleotide, an RNAi reagent such as siRNA, miRNA, shRNA; a guide RNA for a Type 2 CRISPR/Cas effector enzyme), or a small molecule targeting KLRC1/D1 (e.g., with M.W.<1000 Da or <500 Da); optionally, the KLRC1/D1 antagonist is the anti-KLRC1/D1 antibody or antigen-binding portion/fragment thereof.

77. The method of any one of claims 73-76, wherein the antibody is a chimeric antibody, a humanized antibody, or a human antibody.

78. The method of any one of claims 73-77, wherein the antigen-binding portion/fragment is an Fab, Fab′, F(ab′)₂, F_{d}, single chain Fv or scFv, disulfide linked F, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

79. The method of any one of claims 73, 77, and 78, wherein the anti-IGSF8 antibody or antigen-binding portion/fragment thereof specifically binds the D1 (or Ig-V set domain) of IGSF8.

80. The method of any one of claims 73, 77, and 78, wherein the anti-IGSF8 antibody or antigen-binding portion/fragment thereof inhibits IGSF8 binding to KIR3DL1 and/or KIR3DL2.

81. The method of any one of claims 73, 77, and 78, wherein the anti-IGSF8 antibody or antigen-binding portion/fragment thereof inhibits IGSF8 binding to the middle/D2 domain of KIR3DL1 and/or KIR3DL2.

82. The method of any one of claims 73, 77, and 78, wherein the anti-IGSF8 antibody or antigen-binding portion/fragment thereof inhibits IGSF8 binding to S165, I171 and/or M186 of KIR3DL1 and/or KIR3DL2.

83. The method of any one of claims 73, and 77-82, wherein the anti-IGSF8 antibody or antigen-binding portion/fragment thereof is any one of the monoclonal antibody, or antigen-binding portion/fragment thereof according to any one of claims 50-60.

84. The method of any one of claims 74, 75, 77, and 78, wherein the anti-KIR3DL1/2 antibody or antigen-binding portion/fragment thereof, the inhibitory peptide against KIR3DL1/2, the nucleic acid targeting KIR3DL1/2, or the small molecule targeting KIR3DL1/2 binds to an epitope of KIR3DL1/2 comprising residue S165, I171, and/or M186, thereby inhibiting IGSF8 binding to the D2 domain of KIR3DL1/2.

85. The method of any one of claims 74, 75, 77, and 78, wherein the anti-KIR3DL1/2 antibody or antigen-binding portion/fragment thereof specifically binds the middle/D2 Ig-like domain of the ECD of KIR3DL1/2, optionally, the anti-KIR3DL1/2 antibody or antigen-binding portion/fragment thereof specifically binds an epitope comprising residues S165, I171, and/or M186.

86. The method of any one of claims 73-85, wherein the anti-IGSF8 and/or anti-KIR3DL1/2 and/or anti-KLRC1/D1 antibody or antigen-binding portion/fragment thereof is conjugated to a cytotoxic agent.

87. The method of claim 86, wherein the cytotoxic agent is selected from the group consisting of a chemotherapeutic agent, a biologic agent, a toxin, and a radioactive isotope.

88. The method of any one of claims 73-87, wherein the anti-IGSF8 and/or anti-KIR3DL1/2 and/or the anti-KLRC1/D1 antibody or antigen-binding portion/fragment thereof reduces the number of proliferating cells in the cancer and/or reduces the volume or size of a tumor of the cancer.

89. The method of any one of claims 73-88, wherein the anti-IGSF8 and/or anti-KIR3DL1/2 and/or the anti-KLRC1/D1 antibody or antigen-binding portion/fragment thereof is administered in a pharmaceutically acceptable formulation.

90. The method of any one of claims 67-89, wherein the cancer is melanoma (including skin cutaneous melanoma), cervical cancer, lung cancer (e.g., non-small cell lung cancer, lung adenocarcinoma, lung squamous cell carcinoma), colorectal cancer, lymphoma (including B cell lymphoma and DLBCL), leukemia (including CLL and Acute Myeloid Leukemia (AML)), BLCA tumor, breast cancer, head and neck carcinoma, head-neck squamous cell carcinoma, PRAD, THCA, or UCEC, thyroid cancer, unitary tract cancer, uterine cancer, esophagus cancer, liver cancer, ganglia cancer, renal cancer, pancreatic cancer, pancreatic ductal carcinoma, ovarian cancer, prostate cancer, gliomas, glioblastoma, neuroblastoma, thymoma, B-CLL, and a cancer infiltrated with immune cells expressing a receptor to IGSF8.

91. The method of any one of claims 67-89, wherein the cancer is lung cancer, renal cancer, pancreatic cancer, colorectal cancer, acute myeloid leukemia (AML), head and neck carcinoma, liver cancer, ovarian cancer, prostate cancer, or uterine cancer.

92. The method of any one of claims 67-91, wherein the cancer cells and/or tumor immune infiltrating cells in the subject express IGSF8.

93. The method of any one of claims 68, 72, 73, 77-83, and 86-92, wherein the IGSF8 antagonist promotes expression, secretion, or otherwise increases activity of a cytokine or a target gene selected from the group consisting of: CXCL10, CXCL9, TNFα, CD8b, CD8a, Prf1, IFNγ, Gzma, Gzmb, CD274, PDCD1, PDCD1 Ig2, LAG3, Havcr2, Tigit, or CTLA4.

94. The method of claim 93, wherein expression, secretion, or otherwise increased activity of said cytokine or said target gene occurs within tumor microenvironment.

95. The method of claim 93 or 94, wherein expression, secretion, or otherwise increased activity of said cytokine or said target gene is due to immune cell (e.g., T lymphocytes or NK cells) infiltration into tumor microenvironment.

96. The method of any one of claims 68-95, wherein said IGSF8 antagonist, said KIR3DL1 antagonist, said KIR3DL2 antagonist, or said KLRC1/D1 antagonist is an immunostimulatory molecule.

97. The method of claim 96, wherein said IGSF8 antagonist, said KIR3DL1 antagonist, said KIR3DL2 antagonist, or said KLRC1/D1 antagonist stimulates T cell or NK cell activation and/or infiltration into tumor microenvironment.

98. The method of any one of claims 72-97, wherein the immune checkpoint inhibitor is an antibody or antigen-binding fragment thereof specific for PD-1, PD-L1, PD-L2, LAG3, TIGIT, TIM3, NKG2A, CD276, VTCN1, VISR or HHLA2.

99. The method of claim 98, wherein the immune checkpoint inhibitor is an anti-PD-1 antibody, such as cemiplimab, nivolumab, or pembrolizumab.

100. The method of claim 98, wherein the immune checkpoint inhibitor is an anti-PD-L1 antibody, such as avelumab, durvalumab, atezolizumab, KN035, or CK-301.

101. The method of any one of claims 72-97, wherein the immune checkpoint inhibitor is a (non-antibody) peptide inhibitor of PD-1/PD-L1, such as AUNP12; a small molecule inhibitor of PD-L1 such as CA-170, or a macrocyclic peptide such as BMS-986189.

102. The method of any one of claims 72-101, wherein said second therapeutic agent comprises an antibody or an antigen-binding portion/fragment thereof effective to treat a cancer, such as 3F8, 8H9, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Amivantamab, Anatumomab mafenatox, Andecaliximab, Anetumab ravtansine, Anifrolumab, Anrukinzumab, Apolizumab, Aprutumab ixadotin, Arcitumomab, Ascrinvacumab, Aselizumab, Atezolizumab, Atidortoxumab, Atinumab, Atorolimumab, Avelumab, Azintuxizumab vedotin, Bapineuzumab, Basiliximab, Bavituximab, BCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, BirtamimabBivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab, Carlumab, Carotuximab, Catumaxomab, cBR-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, GancotamabGanitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ianalumab, Ibalizumab, IBI308, Ibritumomab tiuxetan, Icrucumab, Idarucizumab, Ifabotuzumab, Igovomab, Iladatuzumab vedotin, IMAB363, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Mapatumumab, Margetuximab, MarstacimabMaslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, OtilimabOtlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, RanevetmabRanibizumab, Raxibacumab, Ravagalimab, Ravulizumab, Refanezumab, Regavirumab, REGN-EB, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Talquetamab, Tamtuvetmab, Tanezumab, Taplitumomab paptox, Tarextumab, TavolimabTeclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, tiragolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab, Zolbetuximab, (=IMAB362, Claudiximab), Zolimomab aritox, or combination thereof.

103. The method of any one of claims 72-102, wherein said second therapeutic agent comprises an antibody or an antigen-binding portion/fragment thereof effective to induce ADCC and/or CDC.

104. The method of any one of claims 66-103, wherein the subject is an animal model of a cancer.

105. Use of an IGSF8 antagonist, an KIR3DL1 antagonist, an KIR3DL2 antagonist, or an KLRC1/D1 antagonist that inhibits IGSF8 binding to a receptor of IGSF8 selected from KIR3DL1, KIR3DL2, and KLRC1/D2 heterodimer, for treating cancer in a subject.

106. The use of claim 105, for combination use with a second therapeutic agent of any one of claims 72 and 98-103.

107. A composition comprising an IGSF8 antagonist, an KIR3DL1 antagonist, an KIR3DL2 antagonist, or an KLRC1/D1 antagonist, that inhibits IGSF8 binding to a receptor of IGSF8 selected from KIR3DL1, KIR3DL2, and KLRC1/D2 heterodimer, for use in any of claims 66-104.

108. An antibody which specifically bind IGSF8 for use in a method of treating cancer, preferably through stimulating T cell and/or NK cell activation.

109. An antibody which specifically bind IGSF8 for use in a method of treating cancer, preferably through combination with a second therapeutic agent of any one of claims 72 and 98-103.

110. A device or kit comprising at least one antibody, monoclonal antibody, heavy or light chain thereof, or antigen-binding portion/fragment thereof, according to any one of claims 50-60, said device or kit optionally comprising a label to detect said at least one antibody, monoclonal antibody, heavy or light chain thereof, or antigen-binding portion/fragment thereof, or a complex comprising said at least one antibody, monoclonal antibody, heavy or light chain thereof, or antigen-binding portion/fragment thereof.

111. A fusion protein comprising an IGSF8 polypeptide and an antibody Fc region.

112. The fusion protein of claim 111, wherein the IGSF8 polypeptide is a full-length IGSF8, an ECD of IGSF8, or the D1 (or Ig V-set) domain of the IGSF8 ECD.

113. The fusion protein of claim 111 or 112, wherein the antibody Fc region is IgG1 Fc region, such as a human IgG1 Fc region.

114. A polynucleotide encoding the fusion protein of any one of claims 111-113.

115. A vector comprising the polynucleotide of claim 114.

116. A host cell comprising the polynucleotide of claim 114, or the vector of claim 115, for expressing the encoded fusion protein.

117. A method of producing the fusion protein of any one of claims 111-113, the method comprising:
(i) culturing the host cell of claim 116 capable of expressing said fusion protein under a condition suitable to express said fusion protein; and
(ii) recovering/isolating/purifying the expressed fusion protein.

118. A method of suppressing activity of a primary NK cell or a T cell, comprising contacting said primary NK cell or said T cell with the fusion protein of any one of claims 111-113.

119. A method of detecting the presence or level of an IGSF8 polypeptide in a sample, the method comprising contacting the IGSF8 polypeptide in the sample with the antibody, monoclonal antibody, or antigen-binding portion/fragment thereof, according to any one of claims 50-60, wherein said antibody, monoclonal antibody, or antigen-binding portion/fragment thereof is labeled by a detectable label, or can be attached to a detectable label.

120. The method of claim 119, wherein said antibody, monoclonal antibody, or antigen binding portion/fragment thereof, forms a complex with the IGSF8 polypeptide, and the complex is detected in the form of an enzyme linked immunosorbent assay (ELISA), radioimmune assay (RIA), immunochemical method, Western blot, or an intracellular flow assay.

121. A method for monitoring the progression of a disorder associated with aberrant (e.g., higher than normal) IGSF8 expression in a subject, the method comprising:
a) detecting, in a sample obtained from the subject, at a first point in time a first level of IGSF8 using the antibody, monoclonal antibody, or antigen-binding portion/fragment thereof, according to any one of claims 50-60;
b) repeating step a) at a subsequent point in time to obtain a second level of IGSF8; and
c) comparing the first and the second levels of IGSF8 detected in steps a) and b),
respectively, to monitor the progression of the disorder in the subject, wherein a higher second level than the first level is indicative that the disease has progressed.

122. The method of claim 121, wherein between the first point in time and the subsequent point in time, the subject has undergone a treatment to ameliorate the disorder.

123. A method for predicting the clinical outcome of a subject afflicted with a disorder associated with aberrant (e.g., higher than normal) IGSF8 expression, the method comprising:
a) determining the level of IGSF8 in a first sample obtained from the subject, using the antibody, monoclonal antibody, or antigen-binding portion/fragment thereof, according to any one of claims 50-60;
b) determining the level of IGSF8 in a second sample obtained from a control subject having a good clinical outcome, using the antibody, monoclonal antibody, or antigen-binding portion/fragment thereof, according to any one of claims 50-60; and
c) comparing the level of IGSF8 in the first and the second samples;
wherein a significantly higher (e.g., >20%, >50% or more increase) level of IGSF8 in the first sample as compared to the level of IGSF8 in the second sample is an indication that the subject has a worse clinical outcome, and/or,
wherein a significantly lower (e.g., >20%, >50% or more decrease) level of IGSF8 in the first sample as compared to the level of IGSF8 in the second sample is an indication that the subject has a better clinical outcome.

124. A method of assessing the efficacy of a therapy for a disorder associated with aberrant (e.g., higher than normal) IGSF8 expression in a subject, the method comprising:
a) determining the level of IGSF8 using the antibody, monoclonal antibody, or antigen-binding portion/fragment thereof, according to any one of claims 50-60, in a first sample obtained from the subject prior to providing at least a portion of the therapy to the subject, and
b) repeat step a) in a second sample obtained from the subject following provision of said portion of the therapy,
wherein a significantly lower (>20%, >50% or more decrease) level of IGSF8 in the second sample, relative to the first sample, is an indication that the therapy is efficacious for inhibiting the disorder in the subject; and/or,
wherein a substantially identical or higher level of IGSF8 in the second sample, relative to the first sample, is an indication that the therapy is not efficacious for inhibiting the disorder in the subject.

125. The method of claim 121 or 122, wherein the disease is cancer.

126. A method of assessing the efficacy of a test compound for inhibiting a disorder associated with aberrant (e.g., higher than normal) IGSF8 expression in a subject, the method comprising:
a) determining the level of IGSF8 using the antibody, monoclonal antibody, or antigen-binding portion/fragment thereof, according to any one of claims 50-60, in a first sample obtained from the subject, wherein the first sample has been exposed to an amount of the test compound; and
b) determining the level of IGSF8 using the antibody, monoclonal antibody, or antigen-binding portion/fragment thereof, according to any one of claims 50-60, in a second sample obtained from the subject, wherein the second sample has not been exposed to the test compound,
wherein a significantly lower (>20%, >50% or more decrease) level of IGSF8 in the first sample relative to that of the second sample, is an indication that the amount of the test compound is efficacious for inhibiting the disorder in the subject, and/or,
wherein a substantially identical level of IGSF8 in the first sample relative to that of the second sample, is an indication that the amount of the test compound is not efficacious for inhibiting the disorder in the subject.

127. The method of claim 126, wherein the first and second samples are portions of a single sample obtained from the subject or portions of pooled samples obtained from the subject.

128. The method of any one of claims 119-127, wherein the disorder is a cancer.

129. The method of claim 128, wherein the cancer is lung cancer, renal cancer, pancreatic cancer, colorectal cancer, Acute myeloid leukemia (AML), head and neck carcinoma, liver cancer, ovarian cancer, prostate cancer, uterine cancer, gliomas, glioblastoma, neuroblastoma, breast cancer, pancreatic ductal carcinoma, thymoma, B-CLL, leukemia, B cell lymphoma, and a cancer infiltrated with immune cells (e.g., T cells and/or NK cells) expressing a receptor to IGSF8 (e.g., KIR3DL1, KIR3DL2, and/or KLRC1/D1).

130. The method of any one of claims 119-129, wherein the sample comprises cells, serum, peritumoral tissue, and/or intratumoral tissue obtained from the subject.

131. The method of any one of claims 121-130, wherein the subject is a human.

132. A method of screening for a functional IGSF8 antagonist, the method comprising contacting a candidate agent (e.g., small molecule, peptide, aptamer, polynucleotide, etc) with a co-culture of NK cells and target cells that express IGSF8 and are resistant to NK cell-mediated cytotoxicity, and identifying the candidate agent that promotes NK cell-mediated cytolytic activity towards the target cell, thereby identifying the candidate agent as an IGSF8 antagonist.

133. A method of screening for a functional IGSF8 antagonist, the method comprising contacting a candidate agent (e.g., small molecule, peptide, aptamer, polynucleotide, etc) with a Jurkat NFAT reporter cell in the presence of T-cell activation signals and IGSF8, wherein the candidate agent is identified as the functional IGSF8 antagonist, when the reporter cell is not activated in the absence of the candidate agent and is activated in the presence of the candidate agent.

134. An antibody which specifically bind KIR3DL1/2 for use in a method of treating cancer, through inhibiting KIR3DL1/2-IGSF8 interaction, thereby stimulating NK cell activation.

135. An antibody which specifically bind KIR3DL1/2 for use in a method of treating cancer, preferably through combination with a second therapeutic agent of any one of claims 72 and 98-103.

136. A monoclonal antibody or an antigen-binding fragment thereof specific for KIR3DL1/2, preferably the second/middle/D2 Ig-like domain of the ECD of KIR3DL1/2, or an epitope comprising residues S165, I171, and/or M186.

137. The monoclonal antibody or antigen-binding fragment thereof of claim 136, which is a human-mouse chimeric antibody, a humanized antibody, a human antibody, a CDR-grafted antibody, or a resurfaced antibody.

138. The monoclonal antibody or antigen-binding fragment thereof of claims 136 or 137, wherein said antigen-binding fragment thereof is an Fab, Fab′, F(ab′)₂, F_{d}, single chain Fv or scFv, disulfide linked F, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

139. The monoclonal antibody or antigen-binding fragment thereof of any one of claims 136-138, wherein said monoclonal antibody or antigen-binding fragment thereof binds KIR3DL1/2 with a K_{d} of less than about 25 nM, 20 nM, 15 nM, 10 nM, 5 nM, 2 nM, or 1 nM.

140. A monoclonal antibody or an antigen-binding fragment thereof, which competes with the monoclonal antibody or antigen-binding fragment thereof of any one of claims 136-139 for binding to KIR3DL1/2.

141. The monoclonal antibody of any one of claims 136-140, wherein the antibody or antigen-binding portion/fragment thereof specifically binds the second/middle/D2 ECD of KIR3DL1/2, preferably with a K_{D} of no more than 5 nM, 2 nM, or 1 nM.

142. The monoclonal antibody of any one of claims 136-141, wherein the antibody or antigen-binding portion/fragment thereof inhibits IGSF8 binding to KIR3DL1/2.

143. A monoclonal antibody or an antigen-binding portion/fragment thereof, which specifically binds the middle/D2 ECD of KIR3DL1/2 (e.g., specifically binds an epitope comprising residues S165, I171, and/or M186), which inhibits IGSF8 binding to KIR3DL1/2.

144. The monoclonal antibody or antigen-binding portion/fragment thereof of claim 143, having a K_{D} of no more than 5 nM, 2 nM, or 1 nM.
